SEQUENCE LISTING (587 sequences)

SEQ ID NO: 1 - DNA, 311 bp, synthetic construct
  source 1..311: mol_type = other DNA
ggcctaactggccggtaccacatcggctatgctgctgctatgcgagcgtcagtattttatctttgatcagctattttatctttagtatcgtattttatctttctcatcgtattttatctttatccgattattttatctttcagcagttattttatctttggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 2 - DNA, 311 bp, synthetic construct
  source 1..311: mol_type = other DNA
ggcctaactggccggtaccagctcatgcctatccgattagcttatcttttgaccagagctagcttatctttctaactcgcatagcttatcttttgcaagctactagcttatctttcgatgctcattagcttatctttagacgtactctagcttatctttggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 3 - DNA, 311 bp, synthetic construct
  source 1..311: mol_type = other DNA
ggcctaactggccggtaccatcactgctgaggtacagatgcacgatgtagctgagcgacagtatagtgcacagtgagtcattatgatacgtgtcattatcaccattgtcattattagacgtgtcattatctgctatgtcattatgctacaggtcattatggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 4 - DNA, 311 bp, synthetic construct
  source 1..311: mol_type = other DNA
ggcctaactggccggtacccagcagtcattatacgtcgcctaaatcgagatgctgtactgatctatattccagatgttttcaattccagatgttttacattccagatgttttacattccagatgtttctcattccagatgttttgaattccagatgtttggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 5 - DNA, 311 bp, synthetic construct
  source 1..311: mol_type = other DNA
ggcctaactggccggtaccctgagcgacagtatagtgcacagtgacattacagatgtttacgacgaattacagatgtttctcatcgattacagatgtttcagctcaattacagatgtttgctgctgattacagatgtttaccagagattacagatgtttggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 6 - DNA, 311 bp, synthetic construct
  source 1..311: mol_type = other DNA
ggcctaactggccggtacccgatgtagctgagcgacagtatagtgcacagtgactgcagcagtcattatacgtcgcctaaatcgagatgctgtactgatctataaggatcggtaatgacgtaatgacgtaatgacgtaatgacgtaatgacgtaatgacggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 7 - DNA, 311 bp, synthetic construct
  source 1..311: mol_type = other DNA
ggcctaactggccggtaccagctgagcgacagtatagtgcacagtgactgcagcagtcattatacgtcgcctaaatcgagatgctgtactgatctataagtcgtaaactgtcgtaaactgtcgtaaactgtcgtaaactgtcgtaaactgtcgtaaactggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 8 - DNA, 311 bp, synthetic construct
  source 1..311: mol_type = other DNA
ggcctaactggccggtacctgtagctgagcgacagtatagtgcacagtgactgcagcagtcattgtcgtaaattgagtatcgtcgtaaattgacgaacgtcgtaaattagcgacagtcgtaaattagtacctgtcgtaaattactctgcgtcgtaaattggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 9 - DNA, 311 bp, synthetic construct
  source 1..311: mol_type = other DNA
ggcctaactggccggtaccatccgatgtgcctgacgaactcatttctaatctatcgatgtagctttctaatctatgcagtcattattctaatctattcgcaatctattctaatctatcttctaactcttctaatctattgctacagctttctaatctatggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 10 - DNA, 311 bp, synthetic construct
  source 1..311: mol_type = other DNA
ggcctaactggccggtaccgcacagtgactgcagcagtcattatacgtcgcctaaatcgagatgctgtactgatctatttcttggcagatgattcttggcagatcgttcttggcagagcattcttggcagaggtttcttggcagactcttcttggcagaggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 11 - DNA, 311 bp, synthetic construct
  source 1..311: mol_type = other DNA
ggcctaactggccggtaccgtgcaccattagtacctgatcagcgatgctcatctcgacctgatcggtacaacttctcacggaggcttctaactcgccgcaattataacgcaattattccgcaattactacgcaattacctcgcaattaactcgcaattaggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 12 - DNA, 311 bp, synthetic construct
  source 1..311: mol_type = other DNA
ggcctaactggccggtaccacatcggctatgctgctgctaatgccacgtcaccacatcgacatgccacgtcaccatcatgccatgccacgtcaccactgcaagatgccacgtcaccacagtataatgccacgtcaccaagttactatgccacgtcaccaggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 13 - DNA, 311 bp, synthetic construct
  source 1..311: mol_type = other DNA
ggcctaactggccggtaccccccaaatcaccccccccccaccgtaaagtccccaaatcaccccccccccaaggtaagacccccaaatcacccccccccccgtcgcctaaccccaaatcacccccccccctactctgctcccccaaatcaccccccccccggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 14 - DNA, 311 bp, synthetic construct
  source 1..311: mol_type = other DNA
ggcctaactggccggtaccgaccgtaaagtggtgtgcaccattgaaacttgagcttacaccatcgaaacttgagcgtatcgcatcgaaacttgagcggtacagatggaaacttgagcaccattagtagaaacttgagcagcgacagtagaaacttgagcggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 15 - DNA, 311 bp, synthetic construct
  source 1..311: mol_type = other DNA
ggcctaactggccggtacctgcacagtgactgcagcagtcgggcgtgcgctcccgactagcccagggcgtgcgctcccgactagccccgggcgtgcgctcccgactagccctgggcgtgcgctcccgactagccccgggcgtgcgctcccgactagcccggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 16 - DNA, 311 bp, synthetic construct
  source 1..311: mol_type = other DNA
ggcctaactggccggtaccaggatcgactagaagtcgcagattagacgacgatacgtactactctgctcctagacgtatcctttgatgtaaatcctttgatgtcaatcctttgatgttaatcctttgatgttagtcctttgatgtctgtcctttgatgtggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 17 - DNA, 311 bp, synthetic construct
  source 1..311: mol_type = other DNA
ggcctaactggccggtacctgagcgacagtatagtgcacagtgactgcagcagtcattatacgtcgcctaaaagacatcaaaggtccagacatcaaaggtacagacatcaaaggggaagacatcaaagggacagacatcaaaggtgcagacatcaaaggggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 18 - DNA, 311 bp, synthetic construct
  source 1..311: mol_type = other DNA
ggcctaactggccggtaccatgcacgatgtagctgagaaacatcaaaggacgcaacgccaaacatcaaaggagcctacacgaaacatcaaagggacgctgctaaaacatcaaaggctacacgaccaaacatcaaagggccttacaccaaacatcaaaggggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 19 - DNA, 298 bp, synthetic construct
  source 1..298: mol_type = other DNA
gaattctagtgcacagtgactgcagcaatgccacgtcaacatcatgccatgccacgtcaacacctacacatgccacgtcaacaaccagagatgccacgtcaacactagcatatgccacgtcaacataaggatatgccacgtcaacaggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 20 - DNA, 298 bp, synthetic construct
  source 1..298: mol_type = other DNA
gaattcgtgcaccattagtacctgatcagcgatgctcatctcgacctgatcggtacaacttctcacggaggcttctaactcgccgcaattataacgcaattattccgcaattactacgcaattacctcgcaattaactcgcaattaggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 21 - DNA, 315 bp, synthetic construct
  source 1..315: mol_type = other DNA
ggcctaacgaattcgacgctgctacagctcagcctacacgaccgtaaagtggtgtgcacaccggaaatgagtatagaccggaaatggccttacaccggaaatgcagctcaaccggaaatgactgcagaccggaaatgcgctgctaccggaaatgggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccaccatggtggcc

SEQ ID NO: 22 - DNA, 311 bp, synthetic construct
  source 1..311: mol_type = other DNA
ggcctaactggccgaattctgagcgacagtatagtgcacagtgactgcagcagtcattatacgtaccggaagtgtgtgcctaccggaagtgctatgcgaccggaagtgtagacgaaccggaagtgcagattaaccggaagtggctgctaaccggaagtgggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 23 - DNA, 298 bp, synthetic construct; identical to SEQ ID NO: 20
  source 1..298: mol_type = other DNA

SEQ ID NO: 24 - DNA, 298 bp, synthetic construct
  source 1..298: mol_type = other DNA
gaattcgtcattatacgtcgcgtcatgcatgactgcctgagcggtcatgcatgactgctactcaagtcatgcatgactgcgaccagagtcatgcatgactgccgcctaagtcatgcatgactgcctctgctgtcatgcatgactgcggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 25 - DNA, 298 bp, synthetic construct
  source 1..298: mol_type = other DNA
gaattcaagtcgcagattagacgacgatacgtactactctgctcctagacgtactcaagtatattaatccagtgaccattaatccactcatgcttaatccaataactgttaatccagtatcgcttaatccactacagcttaatccaggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 26 - DNA, 311 bp, synthetic construct
  source 1..311: mol_type = other DNA
ggcctaactggccgaattccagatgcacgatgtagctgagcgacagtaaactgtaacctgatacagcaactgtaacctgataccctaactgtaacctgatacgataactgtaacctgatacaaaaactgtaacctgatacggcaactgtaacctgatacggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 27 - DNA, 311 bp, synthetic construct
  source 1..311: mol_type = other DNA
ggcctaactggccgaattcgactgcagcagtcattatacgtcgcctaaatcggagaacaaaggatggtgtggagaacaaaggataactgagagaacaaaggaaggatcggagaacaaaggaactgctggagaacaaaggatatagtggagaacaaaggaggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 28 - DNA, 311 bp, synthetic construct
  source 1..311: mol_type = other DNA
ggcctaactggccgaattcctgagcgacagtatagtgcacagtgactgcagcagtcattcctttgatgtacgcaactcctttgatgtctatgcgtcctttgatgttaaggattcctttgatgtaggtacatcctttgatgtccgtaaatcctttgatgtggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 29 - DNA, 298 bp, synthetic construct
  source 1..298: mol_type = other DNA
gaattcaggatcgactagaagtcgcagattagacgacgatacgtactactctgctcctagacgtatcctttgatgtaaatcctttgatgtcaatcctttgatgttaatcctttgatgttagtcctttgatgtctgtcctttgatgtggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 30 - DNA, 311 bp, synthetic construct
  source 1..311: mol_type = other DNA
ggcctaactggccgaattccaagactgcaagctacgtgtgaccagagccgataactgagggcgggaacgcgcaacggggcgggaacgatgctgtgggcgggaacgacagctcgggcgggaacgctctgctgggcgggaacggctcctagggcgggaacgggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 31 - DNA, 298 bp, synthetic construct; identical to SEQ ID NO: 29
  source 1..298: mol_type = other DNA

SEQ ID NO: 32 - DNA, 298 bp, synthetic construct
  source 1..298: mol_type = other DNA
gaattcaggtaagtttcccgccaaaatgtgaccagagtttcccgccaaaatgacgaactcgtttcccgccaaaaatgtagctgagtttcccgccaaaacatagttactgtttcccgccaaaacctaaatcgagtttcccgccaaaaggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 33 - DNA, 298 bp, synthetic construct
  source 1..298: mol_type = other DNA
gaattctgctatgcgagcgtcagctcatgcctatccgatgtgcctatgtaaacataagagccgatgtaaacatataaggatatgtaaacatatagacgaatgtaaacatagaggtacatgtaaacataacacgacatgtaaacataggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 34 - DNA, 298 bp, synthetic construct
  source 1..298: mol_type = other DNA
gaattctacagctcagcctacacgaccgtaaagtggtgtgcaccattgaccccccacaaagcaggaccccccacaaagcgagaccccccacaaaggacgaccccccacaaagcctgaccccccacaaagagtgaccccccacaaagggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 35 - DNA, 298 bp, synthetic construct
  source 1..298: mol_type = other DNA
gaattcaaggtagaccccccactaagctcaagtatagaccccccactaagatagtgcacagaccccccactaagtatccgatgtgaccccccactaagcgcaacgcctgaccccccactaagtcctagacgtgaccccccactaagggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 36 - DNA, 298 bp, synthetic construct
  source 1..298: mol_type = other DNA
gaattcaactgagtatcgcatcgctcaagatcagtggtcataaattagcagtcattgtcataaattcctgatcggtgtcataaattgcctaaatcggtcataaattcagctcatgcgtcataaattacgctgctacgtcataaattggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 37 - DNA, 298 bp, synthetic construct
  source 1..298: mol_type = other DNA
gaattcagtatagtgcacagtgactgcagcagtcattatacgtcgccggggtcaaaggtcaccaggggtcaaaggtcatctggggtcaaaggtcattaggggtcaaaggtcatagggggtcaaaggtcacgaggggtcaaaggtcaggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 38 - DNA, 297 bp, synthetic construct
  source 1..297: mol_type = other DNA
aattcacatcggctatgctgctgctacaggtcaaaggtcattagacgcaggtcaaaggtcacacagtgcaggtcaaaggtcaaggtacacaggtcaaaggtcactgacgacaggtcaaaggtcactcatctcaggtcaaaggtcaggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 39 - DNA, 298 bp, synthetic construct
  source 1..298: mol_type = other DNA
gaattctgcaccattagtacctgatcagcgatgctattttggcgcccaaatcatattttggcgcccaaatgacattttggcgcccaaatacaattttggcgcccaaatacgattttggcgcccaaatagcattttggcgcccaaatggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 40 - DNA, 298 bp, synthetic construct
  source 1..298: mol_type = other DNA
gaattcggtacaacttctcacggaggcttttggcgccatttcgacgatttttggcgccatttactcaagttttggcgccattttagtgcattttggcgccatttcgcaatcttttggcgccatttggaggctttttggcgccatttggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 41 - DNA, 298 bp, synthetic construct
  source 1..298: mol_type = other DNA
gaattcacgatacgtactactctgctcctagacgtactcaagtataaggtaagacatagttaccgcaattataagacacgcaattactagaagcgcaattaacgtcgccgcaattagactgcacgcaattagaatctccgcaattaggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 42 - DNA, 298 bp, synthetic construct
  source 1..298: mol_type = other DNA
gaattcaagtataatgtaaacacggcagcatcgtccaatgtaaacacggcaagacatagtaatgtaaacacggctctcacggagaatgtaaacacggcctagcatcgtaatgtaaacacggcgatgctcatcaatgtaaacacggcggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 43 - DNA, 298 bp, synthetic construct
  source 1..298: mol_type = other DNA
gaattcaagtcgcagattagacgaaaaaccggttatgacgtactcaaaaaccggttatgagatgctgtaaaaccggttattccgacgcaaaaaaccggttatacgaactcataaaaccggttatagctcagcctaaaaccggttatggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 44 - DNA, 298 bp, synthetic construct
  source 1..298: mol_type = other DNA
gaattctgactgcagcagtcattatacgtcgcctaaatcgagatgctgtacgtcgtaaattcacgaccgtcgtaaattcgataacgtcgtaaattctagcatgtcgtaaatttgcagcagtcgtaaattagattaggtcgtaaattggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 45 - DNA, 298 bp, synthetic construct
  source 1..298: mol_type = other DNA
gaattcattagacgacgatacgtactactctgctcctagacgtactcaagtataaggtaagacgcaattattgcacaggcaattattcagcctgcaattatctacagcgcaattatctgatcagcaattatgatacgtgcaattatggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 46 - DNA, 298 bp, synthetic construct
  source 1..298: mol_type = other DNA
gaattcactctgctcctagacgtactcaagtataaggtaggacacgtgcccgatgcacggacacgtgcccccgtaaaggacacgtgccctaaatcgggacacgtgccctagacgtggacacgtgcccgactagaggacacgtgcccggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 47 - DNA, 298 bp, synthetic construct
  source 1..298: mol_type = other DNA
gaattccacagtgactgcagcagtcattatacgtcgcctaaatcgagatgctgtactgatctattaagccgcgtactcttaagccggtcattattaagccgctataagttaagccgcaacgccttaagccgacgaccgttaagccgggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 48 - DNA, 298 bp, synthetic construct
  source 1..298: mol_type = other DNA
gaattctcggctatgctgctgctatgcgagcgtcagctcatgcctatccgatgtgcctgacgaactcatcgacgctgctacagctaatcctatgctaatcctaacctaatcctaccctaatcctagcctaatccttgcctaatcctggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 49 - DNA, 298 bp, synthetic construct
  source 1..298: mol_type = other DNA
gaattctgtactgatctataaggatcgactagaagtcgcagattagtatgtggtttagtacctgtatgtggttttcgcaatgtatgtggtttatgctgcgtatgtggtttagcagtcgtatgtggtttgagcgtcgtatgtggtttggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 50 - DNA, 298 bp, synthetic construct
  source 1..298: mol_type = other DNA
gaattcctgcagcagtcattatacgtcgcctaaatcgagatgctgtactgatctataaggatcgagtatgtggtttatcgtatgtggtttgtagtatgtggtttctggtatgtggttttgtgtatgtggtttccagtatgtggtttggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 51 - DNA, 298 bp, synthetic construct
  source 1..298: mol_type = other DNA
gaattccacgatgtagctgagcgacagtatagtgcacagtgactgcagccaattaactgacgaactccaattaaatcagtgatcccaattaatgcaagctacccaattaatatgctgctgccaattaacatcggctatccaattaaggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 52 - DNA, 298 bp, synthetic construct
  source 1..298: mol_type = other DNA
gaattcttagtacctgatcagcgatgctcatctcgacctgatcggtactcaattaatgtactgatctcaattaagtcgcctaaatcaattaacgtactactctcaattaagatcggtacatcaattaaaagtcgcagatcaattaaggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 53 - DNA, 298 bp, synthetic construct
  source 1..298: mol_type = other DNA
gaattcctacgtgtgaccagagccgataactgagtatcgcatcgctcaagatcagtgatcactgcgaaatttgagccctgaaatttgagccgagaaatttgagcgctgaaatttgagccacgaaatttgagcttagaaatttgagcggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 54 - DNA, 298 bp, synthetic construct
  source 1..298: mol_type = other DNA
gaattcgacctgatcggtacaacttctcacggaggcttctaactctcctttgatataactcgctcctttgatatagcagtctcctttgatatctcatcttcctttgatatctgtacttcctttgatattgctatgtcctttgatatggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 55 - DNA, 522 bp, synthetic construct
  source 1..522: mol_type = other DNA
ggcctaactggccggtaccactagtggtgactcatgggtgactcatgggtgactcatggtgatcatgctagcctcgaggatatcaagatcggtaccacctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgattaccggtgctagcctcgaggatatcaagatctggcctcggcggccaagcttggcaatccggtactgttggtaaagccacc

SEQ ID NO: 56 - DNA, 588 bp, synthetic construct
  source 1..588: mol_type = other DNA
ggcctaactggccggtaccgatcttgatatcctcgaggctagcatgatcaccatgagtcacccatgagtcacccatgagtcacccatgagtcacccatgagtcacccatgagtcacccatgagtcacccatgagtcacccatgagtcaccactagtggtaccacctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgattaccggtgctagcctcgaggatatcaagatctggcctcggcggccaagcttggcaatccggtactgttggtaaagccacc

SEQ ID NO: 57 - DNA, 577 bp, synthetic construct
  source 1..577: mol_type = other DNA
ggcctaactggccggtaccgattcttgatatcctcgaggctagcatgatcaccatgagtcacccatgagtcacccatgagtcacccatgagtcacccatgagtcacccatgagtcacccatgagtcacccatgagtcacccatgagtcaccactagtggtaccgatcttgatatcctcgaggctagcatgatcaccatgagtcacccatgagtcacccatgagtcacccatgagtcacccatgagtcacccatgagtcacccatgagtcacccatgagtcacccatgagtcaccactagtggtaccagtggtgggggagtgaaaagagagatggagaaagaggggatgggcagaaagaggaggaggagtcaggggcagggcatggaggtgggtggggctgggctgccaaagcaggataaatgcacacctgcctgctggtctgggctccctgcctcgggctctcaccctcctctcctgcagctccagctttgtgcttctaccggtgctagcctcgaggatatcaagatctggcctcggcggccaagcttggcaatccggtactgttggtaaagccacc

SEQ ID NO: 58 - DNA, 401 bp, synthetic construct
  source 1..401: mol_type = other DNA
ggcctaactggccggtaccactagtgacgtcaccggaagtaagaaccggaagtatcgaccggaagtagacaccggaagtactaaccggaagtaactaccggaagtatgcaccggaagtagacgtctacgtaagtggtgggggagtgaaaagagagatggagaaagaggggatgggcagaaagaggaggaggagtcaggggcagggcatggaggtgggtggggctgggctgccaaagcaggataaatgcacacctgcctgctggtctgggctccctgcctcgggctctcaccctcctctcctgcagctccagctttgtgctctaccggtgctagcctcgaggatatcaagatctggcctcggcggccaagcttggcaatccggtactgttggtaaagccacc

SEQ ID NO: 59 - DNA, 461 bp, synthetic construct
  source 1..461: mol_type = other DNA
ggcctaactggccggtaccactagtgacgtcaccggaagtaagaaccggaagtatcgaccggaagtagacaccggaagtactaaccggaagtaactaccggaagtatgcaccggaagtagacgtctacgtaggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctttaccggtgctagcctcgaggatatcaagatctggcctcggcggccaagcttggcaatccggtactgttggtaaagccacc

SEQ ID NO: 60 - DNA, 511 bp, synthetic construct
  source 1..511: mol_type = other DNA
ggcctaactggccggtacactagtgacgtcaccggaagtaagaaccggaagtatcgaccggaagtagacaccggaagtactaaccggaagtaactaccggaagtatgcaccggaagtagacgtctacgtacatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgattactagcctcgaggatatcaagatctggcctcggcggccaagcttggcaatccggtactgttggtaaagccacc

SEQ ID NO: 61 - DNA, 321 bp, synthetic construct
  source 1..321: mol_type = other DNA
ggcctaactggccggtaccactagtgacgtcaccggaagtaagaaccggaagtatcgaccggaagtagacaccggaagtactaaccggaagtaactaccggaagtatgcaccggaagtagacgtctacgtaacccacgtgatgctgagaagtactcctgccctaggaagagactcagggcagagggaggaaggacagcagaccagacagtcacagcagccttgacaaaacgttcctggaactaccggtgctagcctcgaggatatcaagatctggcctcggcggccaagcttggcaatccggtactgttggtaaagccacc

SEQ ID NO: 62 - DNA, 376 bp, synthetic construct
  source 1..376: mol_type = other DNA
ggcctaactggccggtaccactagtgacgtcaccggaagtaagaaccggaagtatcgaccggaagtagacaccggaagtactaaccggaagtaactaccggaagtatgcaccggaagtagacgtctacgtacgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctgaaccggtgctagcctcgaggatatcaagatctggcctcggcggccaagcttggcaatccggtactgttggtaaagccacc

SEQ ID NO: 63 - DNA, 547 bp, synthetic construct
  source 1..547: mol_type = other DNA
ggcctaactggccggtaccactagtgacgtcaccggaagtaagaaccggaagtatcgaccggaagtagacaccggaagtactaaccggaagtaactaccggaagtatgcaccggaagtagacgtctacgtactgagcgacagtatagtgcacagtgacattacagatgtttacgacgaattacagatgtttctcatcgattacagatgtttcagctcaattacagatgtttgctgctgattacagatgtttaccagagattacagatgttttacgtaagtggtgggggagtgaaaagagagatggagaaagaggggatgggcagaaagaggaggaggagtcaggggcagggcatggaggtgggtggggctgggctgccaaagcaggataaatgcacacctgcctgctggtctgggctccctgcctcgggctctcaccctcctctcctgcagctccagctttgtgctctaccggtgctagcctcgaggatatcaagatctggcctcggcggccaagcttggcaatccggtactgttggtaaagccacc

SEQ ID NO: 64 - DNA, 588 bp, synthetic construct
  source 1..588: mol_type = other DNA
actagtgacgtcaccggaagtaagaaccggaagtatcgaccggaagtagacaccggaagtactaaccggaagtaactaccggaagtatgcaccggaagtagacgtctacgtactgagcgacagtatagtgcacagtgacattacagatgtttacgacgaattacagatgtttctcatcgattacagatgtttcagctcaattacagatgtttgctgctgattacagatgtttaccagagattacagatgttttacgtaggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctttaccggtgctagcctcgaggatatcaagatctggcctcggcggccaagcttggcaatccggtactgttggtaaagccacc

SEQ ID NO: 65 - DNA, 657 bp, synthetic construct
  source 1..657: mol_type = other DNA
ggcctaactggccggtacactagtgacgtcaccggaagtaagaaccggaagtatcgaccggaagtagacaccggaagtactaaccggaagtaactaccggaagtatgcaccggaagtagacgtctacgtactgagcgacagtatagtgcacagtgacattacagatgtttacgacgaattacagatgtttctcatcgattacagatgtttcagctcaattacagatgtttgctgctgattacagatgtttaccagagattacagatgttttacgtacatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgattactagcctcgaggatatcaagatctggcctcggcggccaagcttggcaatccggtactgttggtaaagccacc

SEQ ID NO: 66 - DNA, 522 bp, synthetic construct
  source 1..522: mol_type = other DNA
ggcctaactggccggtaccactagtgacgtcaccggaagtaagaaccggaagtatcgaccggaagtagacaccggaagtactaaccggaagtaactaccggaagtatgcaccggaagtagacgtctacgtactgagcgacagtatagtgcacagtgacattacagatgtttacgacgaattacagatgtttctcatcgattacagatgtttcagctcaattacagatgtttgctgctgattacagatgtttaccagagattacagatgttttacgtacgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctgaaccggtgctagcctcgaggatatcaagatctggcctcggcggccaagcttggcaatccggtactgttggtaaagccacc

SEQ ID NO: 67 - DNA, 467 bp, synthetic construct
  source 1..467: mol_type = other DNA
ggcctaactggccggtaccactagtgacgtcaccggaagtaagaaccggaagtatcgaccggaagtagacaccggaagtactaaccggaagtaactaccggaagtatgcaccggaagtagacgtctacgtactgagcgacagtatagtgcacagtgacattacagatgtttacgacgaattacagatgtttctcatcgattacagatgtttcagctcaattacagatgtttgctgctgattacagatgtttaccagagattacagatgttttacgtaacccacgtgatgctgagaagtactcctgccctaggaagagactcagggcagagggaggaaggacagcagaccagacagtcacagcagccttgacaaaacgttcctggaactaccggtgctagcctcgaggatatcaagatctggcctcggcggccaagcttggcaatccggtactgttggtaaagccacc

SEQ ID NO: 68 - DNA, 453 bp, synthetic construct
  source 1..453: mol_type = other DNA
ggcctaactggccggtaccactagtgacgtctacgtactgagcgacagtatagtgcacagtgacattacagatgtttacgacgaattacagatgtttctcatcgattacagatgtttcagctcaattacagatgtttgctgctgattacagatgtttaccagagattacagatgttttacgtaagtggtgggggagtgaaaagagagatggagaaagaggggatgggcagaaagaggaggaggagtcaggggcagggcatggaggtgggtggggctgggctgccaaagcaggataaatgcacacctgcctgctggtctgggctccctgcctcgggctctcaccctcctctcctgcagctccagctttgtgctctaccggtgctagcctcgaggatatcaagatctggcctcggcggccaagcttggcaatccggtactgttggtaaagccacc

SEQ ID NO: 69 - DNA, 513 bp, synthetic construct
  source 1..513: mol_type = other DNA
ggcctaactggccggtaccactagtgacgtctacgtactgagcgacagtatagtgcacagtgacattacagatgtttacgacgaattacagatgtttctcatcgattacagatgtttcagctcaattacagatgtttgctgctgattacagatgtttaccagagattacagatgttttacgtaggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctttaccggtgctagcctcgaggatatcaagatctggcctcggcggccaagcttggcaatccggtactgttggtaaagccacc

SEQ ID NO: 70 - DNA, 565 bp, synthetic construct
  source 1..565: mol_type = other DNA
ggcctaactggccggtaccactagtgacgtctacgtactgagcgacagtatagtgcacagtgacattacagatgtttacgacgaattacagatgtttctcatcgattacagatgtttcagctcaattacagatgtttgctgctgattacagatgtttaccagagattacagatgttttacgtacatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgattagctagcctcgaggatatcaagatctggcctcggcggccaagcttggcaatccggtactgttggtaaagccacc

SEQ ID NO: 71 - DNA, 513 bp, synthetic construct
  source 1..513: mol_type = other DNA
ggcctaactggccggtaccactagtgacgtcctgagcgacagtatagtgcacagtgacattacagatgtttacgacgaattacagatgtttctcatcgattacagatgtttcagctcaattacagatgtttgctgctgattacagatgtttaccagagattacagatgtttgacgtctacgtaggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctttaccggtgctagcctcgaggatatcaagatctggcctcggcggccaagcttggcaatccggtactgttggtaaagccacc

SEQ ID NO: 72 - DNA, 599 bp, synthetic construct
  source 1..599: mol_type = other DNA
ggcctaactggccggtaccactagtgacgtcctgagcgacagtatagtgcacagtgacattacagatgtttacgacgaattacagatgtttctcatcgattacagatgtttcagctcaattacagatgtttgctgctgattacagatgtttaccagagattacagatgtttgacgtctacgtactgatcagcgatgctcatctcgacctgatcggtacaacttctcacggaggcttctaagtcattacatacgtagtcattactatacgtgtcattacagatgctgtcattacacgaactgtcattacgtactcagtcattactacgtaagtggtgggggagtgaaaagagagatggagaaagaggggatgggcagaaagaggaggaggagtcaggggcagggcatggaggtgggtggggctgggctgccaaagcaggataaatgcacacctgcctgctggtctgggctccctgcctcgggctctcaccctcctctcctgcagctccagctttgtgctctaccggtgctagcctcgaggatatcaagatctggcctcggcggccaagcttggcaatccggtactgttggtaaagccacc

SEQ ID NO: 73 - DNA, 428 bp, synthetic construct
  source 1..428: mol_type = other DNA
ggcctaactggccggtaccactagtgacgtctacgtactgagcgacagtatagtgcacagtgacattacagatgtttacgacgaattacagatgtttctcatcgattacagatgtttcagctcaattacagatgtttgctgctgattacagatgtttaccagagattacagatgttttacgtacgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctgaaccggtgctagcctcgaggatatcaagatctggcctcggcggccaagcttggcaatccggtactgttggtaaagccacc

SEQ ID NO: 74 - DNA, 373 bp, synthetic construct
  source 1..373: mol_type = other DNA
ggcctaactggccggtaccactagtgacgtctacgtactgagcgacagtatagtgcacagtgacattacagatgtttacgacgaattacagatgtttctcatcgattacagatgtttcagctcaattacagatgtttgctgctgattacagatgtttaccagagattacagatgttttacgtaacccacgtgatgctgagaagtactcctgccctaggaagagactcagggcagagggaggaaggacagcagaccagacagtcacagcagccttgacaaaacgttcctggaactaccggtgctagcctcgaggatatcaagatctggcctcggcggccaagcttggcaatccggtactgttggtaaagccacc

SEQ ID NO: 75 - DNA, 565 bp, synthetic construct
  source 1..565: mol_type = other DNA
ggcctaactggccggtaccactagtgacgtcctgagcgacagtatagtgcacagtgacattacagatgtttacgacgaattacagatgtttctcatcgattacagatgtttcagctcaattacagatgtttgctgctgattacagatgtttaccagagattacagatgtttgacgtctacgtacatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgattagctagcctcgaggatatcaagatctggcctcggcggccaagcttggcaatccggtactgttggtaaagccacc

SEQ ID NO: 76 - DNA, 373 bp, synthetic construct
  source 1..373: mol_type = other DNA
ggcctaactggccggtaccactagtgacgtcctgagcgacagtatagtgcacagtgacattacagatgtttacgacgaattacagatgtttctcatcgattacagatgtttcagctcaattacagatgtttgctgctgattacagatgtttaccagagattacagatgtttgacgtctacgtaacccacgtgatgctgagaagtactcctgccctaggaagagactcagggcagagggaggaaggacagcagaccagacagtcacagcagccttgacaaaacgttcctggaactaccggtgctagcctcgaggatatcaagatctggcctcggcggccaagcttggcaatccggtactgttggtaaagccacc

SEQ ID NO: 77 - DNA, 428 bp, synthetic construct
  source 1..428: mol_type = other DNA
ggcctaactggccggtaccactagtgacgtcctgagcgacagtatagtgcacagtgacattacagatgtttacgacgaattacagatgtttctcatcgattacagatgtttcagctcaattacagatgtttgctgctgattacagatgtttaccagagattacagatgtttgacgtctacgtacgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctgaaccggtgctagcctcgaggatatcaagatctggcctcggcggccaagcttggcaatccggtactgttggtaaagccacc

SEQ ID NO: 78 - DNA, 659 bp, synthetic construct
  source 1..659: mol_type = other DNA
ggcctaactggccggtaccactagtgacgtcctgagcgacagtatagtgcacagtgacattacagatgtttacgacgaattacagatgtttctcatcgattacagatgtttcagctcaattacagatgtttgctgctgattacagatgtttaccagagattacagatgtttgacgtctacgtactgatcagcgatgctcatctcgacctgatcggtacaacttctcacggaggcttctaagtcattacatacgtagtcattactatacgtgtcattacagatgctgtcattacacgaactgtcattacgtactcagtcattactacgtaggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctttaccggtgctagcctcgaggatatcaagatctggcctcggcggccaagcttggcaatccggtactgttggtaaagccacc

SEQ ID NO: 79 - DNA, 755 bp, synthetic construct
  source 1..755: mol_type = other DNA
ggcctaactggccggtaccacactagtgacgtcctgagcgacagtatagtgcacagtgacattacagatgtttacgacgaattacagatgtttctcatcgattacagatgtttcagctcaattacagatgtttgctgctgattacagatgtttaccagagattacagatgtttgacgtctacgtactgatcagcgatgctcatctcgacctgatcggtacaacttctcacggaggcttctaagtcattacatacgtagtcattactatacgtgtcattacagatgctgtcattacacgaactgtcattacgtactcagtcattactacgtacatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggttaagtggtgggggagtgaaaagagagatggagaaagaggggatgggcagaaagaggaggaggagtcaggggcagggcatggaggtgggtggggctgggctgccaaagcaggataaatgcacacctgcctgctggtctgggctccctgcctcgggctctcaccctcctctcctgcagctccagctttgtgctctaccggtgctagcctcgaggatatcaagatctggcctcggcggccaagcttggcaatccggtactgttggtaaagccacc

SEQ ID NO: 80 - DNA, 683 bp, synthetic construct
  source 1..683: mol_type = other DNA
ggcctaactggccggtacaactagtgactcctttgatgtacgcaactcctttgatgtctatgcgtcctttgatgttaaggattcctttgatgtaggtacatcctttgatgtccgtaaatcctttgatgtggtaccgtctactacctgatcaaacatgcccggacatgtcgtaagacataaacatgcccggacatgtcctcgcaatctaacatgcccggacatgtcctcgcaatctaacatgcccggacatgtctgcaagctacaacatgcccggacatgtcgtactcagtcattactacgtacatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgattactagcctcgaggatatcaagatctggcctcggcggccaagcttggcaatccggtactgttggtaaagccacc

SEQ ID NO: 81 - DNA, 519 bp, synthetic construct
  source 1..519: mol_type = other DNA
ggcctaactggccggtaccactagtgacgtcctgagcgacagtatagtgcacagtgacattacagatgtttacgacgaattacagatgtttctcatcgattacagatgtttcagctcaattacagatgtttgctgctgattacagatgtttaccagagattacagatgtttgacgtctacgtactgatcagcgatgctcatctcgacctgatcggtacaacttctcacggaggcttctaagtcattacatacgtagtcattactatacgtgtcattacagatgctgtcattacacgaactgtcattacgtactcagtcattactacgtaacccacgtgatgctgagaagtactcctgccctaggaagagactcagggcagagggaggaaggacagcagaccagacagtcacagcagccttgacaaaacgttcctggaactaccggtgctagcctcgaggatatcaagatctggcctcggcggccaagcttggcaatccggtactgttggtaaagccacc

SEQ ID NO: 82 - DNA, 574 bp, synthetic construct
  source 1..574: mol_type = other DNA
ggcctaactggccggtaccactagtgacgtcctgagcgacagtatagtgcacagtgacattacagatgtttacgacgaattacagatgtttctcatcgattacagatgtttcagctcaattacagatgtttgctgctgattacagatgtttaccagagattacagatgtttgacgtctacgtactgatcagcgatgctcatctcgacctgatcggtacaacttctcacggaggcttctaagtcattacatacgtagtcattactatacgtgtcattacagatgctgtcattacacgaactgtcattacgtactcagtcattactacgtacgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctgaaccggtgctagcctcgaggatatcaagatctggcctcggcggccaagcttggcaatccggtactgttggtaaagccacc

SEQ ID NO: 83 - DNA, 512 bp, synthetic construct
  source 1..512: mol_type = other DNA
ggcctaactggccggtaccactagtgacgtctgtagctgagcgacagtatagtgcacagtgactgcagcagtcattgtcgtaaattgagtatcgtcgtaaattgacgaacgtcgtaaattagcgacagtcgtaaattagtacctgtcgtaaattactctgcgtcgtaaattgacgtctacgtaggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctttaccggtctagcctcgaggatatcaagatctggcctcggcggccaagcttggcaatccggtactgttggtaaagccacc

SEQ ID NO: 84 - DNA, 453 bp, synthetic construct
  source 1..453: mol_type = other DNA
ggcctaactggccggtaccactagtgacgtctacgtactgatcagcgatgctcatctcgacctgatcggtacaacttctcacggaggcttctaagtcattacatacgtagtcattactatacgtgtcattacagatgctgtcattacacgaactgtcattacgtactcagtcattactacgtaagtggtgggggagtgaaaagagagatggagaaagaggggatgggcagaaagaggaggaggagtcaggggcagggcatggaggtgggtggggctgggctgccaaagcaggataaatgcacacctgcctgctggtctgggctccctgcctcgggctctcaccctcctctcctgcagctccagctttgtgctctaccggtgctagcctcgaggatatcaagatctggcctcggcggccaagcttggcaatccggtactgttggtaaagccacc

SEQ ID NO: 85 - DNA, 513 bp, synthetic construct
  source 1..513: mol_type = other DNA
ggcctaactggccggtaccactagtgacgtctacgtactgatcagcgatgctcatctcgacctgatcggtacaacttctcacggaggcttctaagtcattacatacgtagtcattactatacgtgtcattacagatgctgtcattacacgaactgtcattacgtactcagtcattactacgtaggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctttaccggtgctagcctcgaggatatcaagatctggcctcggcggccaagcttggcaatccggtactgttggtaaagccacc

SEQ ID NO: 86 - DNA, 373 bp, synthetic construct
  source 1..373: mol_type = other DNA
ggcctaactggccggtaccactagtgacgtctacgtactgatcagcgatgctcatctcgacctgatcggtacaacttctcacggaggcttctaagtcattacatacgtagtcattactatacgtgtcattacagatgctgtcattacacgaactgtcattacgtactcagtcattactacgtaacccacgtgatgctgagaagtactcctgccctaggaagagactcagggcagagggaggaaggacagcagaccagacagtcacagcagccttgacaaaacgttcctggaactaccggtgctagcctcgaggatatcaagatctggcctcggcggccaagcttggcaatccggtactgttggtaaagccacc

SEQ ID NO: 87 - DNA, 565 bp, synthetic construct
  source 1..565: mol_type = other DNA
ggcctaactggccggtaccactagtgacgtctacgtactgatcagcgatgctcatctcgacctgatcggtacaacttctcacggaggcttctaagtcattacatacgtagtcattactatacgtgtcattacagatgctgtcattacacgaactgtcattacgtactcagtcattactacgtacatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgattagctagcctcgaggatatcaagatctggcctcggcggccaagcttggcaatccggtactgttggtaaagccacc

SEQ ID NO: 88 - DNA, 453 bp, synthetic construct
  source 1..453: mol_type = other DNA
ggcctaactggccggtaccactagtgacgtctgtagctgagcgacagtatagtgcacagtgactgcagcagtcattgtcgtaaattgagtatcgtcgtaaattgacgaacgtcgtaaattagcgacagtcgtaaattagtacctgtcgtaaattactctgcgtcgtaaattgacgtctacgtaagtggtgggggagtgaaaagagagatggagaaagaggggatgggcagaaagaggaggaggagtcaggggcagggcatggaggtgggtggggctgggctgccaaagcaggataaatgcacacctgcctgctggtctgggctccctgcctcgggctctcaccctcctctcctgcagctccagctttgtgctctaccggtgctagcctcgaggatatcaagatctggcctcggcggccaagcttggcaatccggtactgttggtaaagccacc

SEQ ID NO: 89 - DNA, 428 bp, synthetic construct
  source 1..428: mol_type = other DNA
ggcctaactggccggtaccactagtgacgtctgtagctgagcgacagtatagtgcacagtgactgcagcagtcattgtcgtaaattgagtatcgtcgtaaattgacgaacgtcgtaaattagcgacagtcgtaaattagtacctgtcgtaaattactctgcgtcgtaaattgacgtctacgtacgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctgaaccggtgctagcctcgaggatatcaagatctggcctcggcggccaagcttggcaatccggtactgttggtaaagccacc

SEQ ID NO: 90 - DNA, 564 bp, synthetic construct
  source 1..564: mol_type = other DNA
ggcctaactggccggtacaactagtgacgtctgtagctgagcgacagtatagtgcacagtgactgcagcagtcattgtcgtaaattgagtatcgtcgtaaattgacgaacgtcgtaaattagcgacagtcgtaaattagtacctgtcgtaaattactctgcgtcgtaaattgacgtctacgtacatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgattactagcctcgaggatatcaagatctggcctcggcggccaagcttggcaatccggtactgttggtaaagccacc

SEQ ID NO: 91 - DNA, 373 bp, synthetic construct
  source 1..373: mol_type = other DNA
ggcctaactggccggtaccactagtgacgtctgtagctgagcgacagtatagtgcacagtgactgcagcagtcattgtcgtaaattgagtatcgtcgtaaattgacgaacgtcgtaaattagcgacagtcgtaaattagtacctgtcgtaaattactctgcgtcgtaaattgacgtctacgtaacccacgtgatgctgagaagtactcctgccctaggaagagactcagggcagagggaggaaggacagcagaccagacagtcacagcagccttgacaaaacgttcctggaactaccggtgctagcctcgaggatatcaagatctggcctcggcggccaagcttggcaatccggtactgttggtaaagccacc

SEQ ID NO: 92 - DNA, 428 bp, synthetic construct
  source 1..428: mol_type = other DNA
ggcctaactggccggtaccactagtgacgtctacgtactgatcagcgatgctcatctcgacctgatcggtacaacttctcacggaggcttctaagtcattacatacgtagtcattactatacgtgtcattacagatgctgtcattacacgaactgtcattacgtactcagtcattactacgtacgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctgaaccggtgctagcctcgaggatatcaagatctggcctcggcggccaagcttggcaatccggtactgttggtaaagccacc

SEQ ID NO: 93 - DNA, 605 bp, synthetic construct
  source 1..605: mol_type = other DNA
ggcctaactggccggtaccactagtgacgtctgtagctgagcgacagtatagtgcacagtgactgcagcagtcattgtcgtaaattgagtatcgtcgtaaattgacgaacgtcgtaaattagcgacagtcgtaaattagtacctgtcgtaaattactctgcgtcgtaaattgacgtctacgtaacatcggctatgctgctgctaatgccacgtcaccacatcgacatgccacgtcaccatcatgccatgccacgtcaccactgcaagatgccacgtcaccacagtataatgccacgtcaccaagttactatgccacgtcaccaggtacctacgtaagtggtgggggagtgaaaagagagatggagaaagaggggatgggcagaaagaggaggaggagtcaggggcagggcatggaggtgggtggggctgggctgccaaagcaggataaatgcacacctgcctgctggtctgggctccctgcctcgggctctcaccctcctctcctgcagctccagctttgtgctctaccggtgctagcctcgaggatatcaagatctggcctcggcggccaagcttggcaatccggtactgttggtaaagccacc

SEQ ID NO: 94 - DNA, 663 bp, synthetic construct
  source 1..663: mol_type = other DNA
ggcctaactggccggtacactagtgacgtctgtagctgagcgacagtatagtgcacagtgactgcagcagtcattgtcgtaaattgagtatcgtcgtaaattgacgaacgtcgtaaattagcgacagtcgtaaattagtacctgtcgtaaattactctgcgtcgtaaattgacgtctacgtaacatcggctatgctgctgctaatgccacgtcaccacatcgacatgccacgtcaccatcatgccatgccacgtcaccactgcaagatgccacgtcaccacagtataatgccacgtcaccaagttactatgccacgtcaccaggtacctacgtaggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctttaccggtctagcctcgaggatatcaagatctggcctcggcggccaagcttggcaatccggtactgttggtaaagccacc

SEQ ID NO: 95 - DNA, 716 bp, synthetic construct
  source 1..716: mol_type = other DNA
ggcctaactggccggtacaactagtgacgtctgtagctgagcgacagtatagtgcacagtgactgcagcagtcattgtcgtaaattgagtatcgtcgtaaattgacgaacgtcgtaaattagcgacagtcgtaaattagtacctgtcgtaaattactctgcgtcgtaaattgacgtctacgtaacatcggctatgctgctgctaatgccacgtcaccacatcgacatgccacgtcaccatcatgccatgccacgtcaccactgcaagatgccacgtcaccacagtataatgccacgtcaccaagttactatgccacgtcaccaggtacctacgtacatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgattactagcctcgaggatatcaagatctggcctcggcggccaagcttggcaatccggtactgttggtaaagccacc

SEQ ID NO: 96 - DNA, 525 bp, synthetic construct
  source 1..525: mol_type = other DNA
ggcctaactggccggtaccactagtgacgtctgtagctgagcgacagtatagtgcacagtgactgcagcagtcattgtcgtaaattgagtatcgtcgtaaattgacgaacgtcgtaaattagcgacagtcgtaaattagtacctgtcgtaaattactctgcgtcgtaaattgacgtctacgtaacatcggctatgctgctgctaatgccacgtcaccacatcgacatgccacgtcaccatcatgccatgccacgtcaccactgcaagatgccacgtcaccacagtataatgccacgtcaccaagttactatgccacgtcaccaggtacctacgtaacccacgtgatgctgagaagtactcctgccctaggaagagactcagggcagagggaggaaggacagcagaccagacagtcacagcagccttgacaaaacgttcctggaactaccggtgctagcctcgaggatatcaagatctggcctcggcggccaagcttggcaatccggtactgttggtaaagccacc

SEQ ID NO: 97 - DNA, 580 bp, synthetic construct
  source 1..580: mol_type = other DNA
ggcctaactggccggtaccactagtgacgtctgtagctgagcgacagtatagtgcacagtgactgcagcagtcattgtcgtaaattgagtatcgtcgtaaattgacgaacgtcgtaaattagcgacagtcgtaaattagtacctgtcgtaaattactctgcgtcgtaaattgacgtctacgtaacatcggctatgctgctgctaatgccacgtcaccacatcgacatgccacgtcaccatcatgccatgccacgtcaccactgcaagatgccacgtcaccacagtataatgccacgtcaccaagttactatgccacgtcaccaggtacctacgtacgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctgaaccggtgctagcctcgaggatatcaagatctggcctcggcggccaagcttggcaatccggtactgttggtaaagccacc

SEQ ID NO: 98 - DNA, 459 bp, synthetic construct
  source 1..459: mol_type = other DNA
ggcctaactggccggtaccactagtgacgtctacgtaacatcggctatgctgctgctaatgccacgtcaccacatcgacatgccacgtcaccatcatgccatgccacgtcaccactgcaagatgccacgtcaccacagtataatgccacgtcaccaagttactatgccacgtcaccaggtacctacgtaagtggtgggggagtgaaaagagagatggagaaagaggggatgggcagaaagaggaggaggagtcaggggcagggcatggaggtgggtggggctgggctgccaaagcaggataaatgcacacctgcctgctggtctgggctccctgcctcgggctctcaccctcctctcctgcagctccagctttgtgctctaccggtgctagcctcgaggatatcaagatctggcctcggcggccaagcttggcaatccggtactgttggtaaagccacc

SEQ ID NO: 99 - DNA, 570 bp, synthetic construct
  source 1..570: mol_type = other DNA
ggcctaactggccggtacaactagtgacgtctacgtaacatcggctatgctgctgctaatgccacgtcaccacatcgacatgccacgtcaccatcatgccatgccacgtcaccactgcaagatgccacgtcaccacagtataatgccacgtcaccaagttactatgccacgtcaccaggtacctacgtacatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgattactagcctcgaggatatcaagatctggcctcggcggccaagcttggcaatccggtactgttggtaaagccacc

SEQ ID NO: 100 - DNA, 518 bp, synthetic construct
  source 1..518: mol_type = other DNA
ggcctaactggccggtaccactagtgacgtctacgtaacatcggctatgctgctgctaatgccacgtcaccacatcgacatgccacgtcaccatcatgccatgccacgtcaccactgcaagatgccacgtcaccacagtataatgccacgtcaccaagttactatgccacgtcaccaggtacctacgtaggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctttaccggtctagcctcgaggatatcaagatctggcctcggcggccaagcttggcaatccggtactgttggtaaagccacc

SEQ ID NO: 101 - DNA, 379 bp, synthetic construct
  source 1..379: mol_type = other DNA
ggcctaactggccggtaccactagtgacgtctacgtaacatcggctatgctgctgctaatgccacgtcaccacatcgacatgccacgtcaccatcatgccatgccacgtcaccactgcaagatgccacgtcaccacagtataatgccacgtcaccaagttactatgccacgtcaccaggtacctacgtaacccacgtgatgctgagaagtactcctgccctaggaagagactcagggcagagggaggaaggacagcagaccagacagtcacagcagccttgacaaaacgttcctggaactaccggtgctagcctcgaggatatcaagatctggcctcggcggccaagcttggcaatccggtactgttggtaaagccacc

SEQ ID NO: 102 - DNA, 434 bp, synthetic construct
  source 1..434: mol_type = other DNA
ggcctaactggccggtaccactagtgacgtctacgtaacatcggctatgctgctgctaatgccacgtcaccacatcgacatgccacgtcaccatcatgccatgccacgtcaccactgcaagatgccacgtcaccacagtataatgccacgtcaccaagttactatgccacgtcaccaggtacctacgtacgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctgaaccggtgctagcctcgaggatatcaagatctggcctcggcggccaagcttggcaatccggtactgttggtaaagccacc

SEQ ID NO: 103 - DNA, 317 bp, synthetic construct
  source 1..317: mol_type = other DNA
gaattcaagactgcaagcgagcgacagtatagtgcacagtgactgcagcagtcattatacgtcgcctaaatcgagatgctgtaggcacgtgtatctggcacgtgtactcggcacgtgtactaggcacgtgtaagaggcacgtgtacgcggcacgtgtaggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccaccatggaag

SEQ ID NO: 104 - DNA, 274 bp, synthetic construct
  source 1..274: mol_type = other DNA
gaattcaagactgcaagcgagcgacagtatagtgcacagtgactgcagcagtcattatacgtcgcctaaatcgagatgctgtaggcacgtgtatctggcacgtgtactcggcacgtgtactaggcacgtgtaagaggcacgtgtacgcggcacgtgtaggtacctataaaaggccagcagcagcctgaccacatctcatccgctagcctcgaggatatcaagatctggcctcggcggccaagcttggcaatccggtactgttggtaaagccacc

SEQ ID NO: 105 - DNA, 310 bp, synthetic construct
  source 1..310: mol_type = other DNA
gaattcaagactgcaagcctgagcgacagtatagtgcacagtgactgcagcagtcattatacgtcgcctaaatcgagatgctggacacgtgtccgagacacgtgtctgtgacacgtgtccgggacacgtgtcgcagacacgtgtcgtggacacgtgtcggtacctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 106 - DNA, 274 bp, synthetic construct
  source 1..274: mol_type = other DNA
gaattcaagactgcaagcctgagcgacagtatagtgcacagtgactgcagcagtcattatacgtcgcctaaatcgagatgctggacacgtgtccgagacacgtgtctgtgacacgtgtccgggacacgtgtcgcagacacgtgtcgtggacacgtgtcggtacctataaaaggccagcagcagcctgaccacatctcatccgctagcctcgaggatatcaagatctggcctcggcggccaagcttggcaatccggtactgttggtaaagccacc

SEQ ID NO: 107 - DNA, 764 bp, synthetic construct
  source 1..764: mol_type = other DNA
ggcctaactggccggtaccactagtatcgatccttcatagggcagggaggggtgggcacttgggtgtgaccaaggagaggaggcgcgcctggtcaacagctctccctggcccgtgtccagctccctcctcacacagagaggggggcgcatctcagggatggcatctttcccccccacagggaaattcttatctttgaaacagcatgggaatcgaggcacccaggaggggagcagaggcaggcaggcctccttcaggcccatcctccagctgggctggtggtgccagggaggctccctgcttggtaacaaaggcctgagggagagttgcgaaacccagcaggaaagccggctcaccttcgcctccccctgcggctgggaggagaggaaatatcccatggctgactgtgccaaggaggtgtctgagccagccctcccggcccgagggcagggcaggtggccctgagagataagccaatcccgcagctgcagatgaggagttctgagaagcattgctcaggacagcggtaaatcacttcttggaggtgccctgcacgccggtcctgggagcaggcggcctcccgggggtgcgggagccccactcctccgtggtgtgttccatttgcttcccacatctggaggagctgacgtgccagcctcccccagcaccacccagggacgggaggcaaccggtgctagcctcgaggatatcaagatctggcctcggcggccaagcttggcaatccggtactgttggtaaagccacc

SEQ ID NO: 108 - DNA, 283 bp, synthetic construct
  source 1..283: mol_type = other DNA
ggcctaactggccggtaccgacgtctacctgatcaaacatgcccggacatgtcgtaagacataaacatgcccggacatgtcctcgcaatctaacatgcccggacatgtcctcgcaatctaacatgcccggacatgtctgcaagctacaacatgcccggacatgtctacgtagctagctataaaaggccagcagcagcctgaccacatctcatcctcctcgaggatatcaagatctggcctcggcggccaagcttggcaatccggtactgttggtaaagccacc

SEQ ID NO: 109 - DNA, 283 bp, synthetic construct
  source 1..283: mol_type = other DNA
ggcctaactggccggtaccgacgtccctgatcggtacaacttctcacaacatgcctgggcatgtcgctatgcaacatgcctgggcatgtcagatgcaaacatgcctgggcatgtcctgctataacatgcctgggcatgtcctgctataacatgcctgggcatgtctacgtagctagctataaaaggccagcagcagcctgaccacatctcatcctcctcgaggatatcaagatctggcctcggcggccaagcttggcaatccggtactgttggtaaagccacc

SEQ ID NO: 110 - DNA, 283 bp, synthetic construct
  source 1..283: mol_type = other DNA
ggcctaactggccggtaccgacgtctcgggcaagcgctcccgacatgcccgggcaagcgctcccgacatgcccgggcaagcgctcccgacatgcccgggcaagcgctcccgacatgcccgggcaagcgctcccgacatgcccgggcaagcgctcccgacatgccctacgtagctagctataaaaggccagcagcagcctgaccacatctcatcctcctcgaggatatcaagatctggcctcggcggccaagcttggcaatccggtactgttggtaaagccacc

SEQ ID NO: 111 - DNA, 435 bp, synthetic construct
  source 1..435: mol_type = other DNA
ggcctaactggccggtaccttttgataaaaatcattaggtacggccgcggtgccagggcgtgcccttgggctccccgggcgcgaaactagtgacgtcctgagcgacagtatagtgcacagtgactgcagcagtcattcctttgatgtacgcaactcctttgatgtctatgcgtcctttgatgttaaggattcctttgatgtaggtacatcctttgatgtccgtaaatcctttgatgtgacgtctacgtaggtgactcatgggtgactcatgtacgtaacgcgtcccgacatgccccgcggcgcgccattaaccgccagatttgagtcgcgggacccgttggcagaggtgggaattcaccggtgctagcctcgaggatatcaagatctggcctcggcggccaagcttggcaatccggtactgttggtaaagccacc

SEQ ID NO: 112 - DNA, 434 bp, synthetic construct
  source 1..434: mol_type = other DNA
ggcctaactggccggtaccttttgataaaaatcattaggtacggccgcggtgccagggcgtgcccttgggctccccgggcgcgaaactagtgacgtcggtgactcatgggtgactcatgacgtctacgtactgagcgacagtatagtgcacagtgactgcagcagtcattcctttgatgtacgcaactcctttgatgtctatgcgtcctttgatgttaaggattcctttgatgtaggtacatcctttgatgtccgtaaatcctttgatgttacgtaacgcgtcccgacatgccccgcggcgcgccattaaccgccagatttgagtcgcgggacccgttggcagaggtgggaattcaccggtgctagcctcgaggatatcaagatctggcctcggcggccaagcttggcaatccggtactgttggtaaagccacc

SEQ ID NO: 113 - DNA, 594 bp, synthetic construct
  source 1..594: mol_type = other DNA
ggcctaactggccggtaccaactagtgacgtcctgagcgacagtatagtgcacagtgactgcagcagtcattcctttgatgtacgcaactcctttgatgtctatgcgtcctttgatgttaaggattcctttgatgtaggtacatcctttgatgtccgtaaatcctttgatgtgacgtctacgtaggtgactcatgggtgactcatgtacgtacatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgattagctagcctcgaggatatcaagatctggcctcggcggccaagcttggcaatccggtactgttggtaaagccacc

SEQ ID NO: 114 - DNA, 594 bp, synthetic construct
  source 1..594: mol_type = other DNA
ggcctaactggccggtaccaactagtgacgtcggtgactcatgggtgactcatggacgtctacgtactgagcgacagtatagtgcacagtgactgcagcagtcattcctttgatgtacgcaactcctttgatgtctatgcgtcctttgatgttaaggattcctttgatgtaggtacatcctttgatgtccgtaaatcctttgatgttacgtacatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgattagctagcctcgaggatatcaagatctggcctcggcggccaagcttggcaatccggtactgttggtaaagccacc

SEQ ID NO: 115 - DNA, 566 bp, synthetic construct
  source 1..566: mol_type = other DNA
ggcctaactggccggtaccaactagtgacgtcctgagcgacagtatagtgcacagtgactgcagcagtcattcctttgatgtacgcaactcctttgatgtctatgcgtcctttgatgttaaggattcctttgatgtaggtacatcctttgatgtccgtaaatcctttgatgtgacgtctacgtacatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgattagctagcctcgaggatatcaagatctggcctcggcggccaagcttggcaatccggtactgttggtaaagccacc

SEQ ID NO: 116 - DNA, 311 bp, synthetic construct
  source 1..311: mol_type = other DNA
caactagtgacgtcctgagcgacagtatagtgcacagtgactgcagcagtcattcctttgatgtacgcaactcctttgatgtctatgcgtcctttgatgttaaggattcctttgatgtaggtacatcctttgatgtccgtaaatcctttgatgtgacgtctacgtaggtgactcatgggtgactcatgtacgtaacccacgtgatgctgagaagtactcctgccctaggaagagactcagggcagagggaggaaggacagcagaccagacagtcacagcagccttgacaaaacgttcctggaactaccggt

SEQ ID NO: 117 - DNA, 374 bp, synthetic construct
  source 1..374: mol_type = other DNA
ggcctaactggccggtaccaactagtgacgtcctgagcgacagtatagtgcacagtgactgcagcagtcattcctttgatgtacgcaactcctttgatgtctatgcgtcctttgatgttaaggattcctttgatgtaggtacatcctttgatgtccgtaaatcctttgatgtgacgtctacgtaacccacgtgatgctgagaagtactcctgccctaggaagagactcagggcagagggaggaaggacagcagaccagacagtcacagcagccttgacaaaacgttcctggaactaccggtgctagcctcgaggatatcaagatctggcctcggcggccaagcttggcaatccggtactgttggtaaagccacc

SEQ ID NO: 118 - DNA, 343 bp, synthetic construct
  source 1..343: mol_type = other DNA
aactagtgacgtcctgagcgacagtatagtgcacagtgactgcagcagtcattcctttgatgtacgcaactcctttgatgtctatgcgtcctttgatgttaaggattcctttgatgtaggtacatcctttgatgtccgtaaatcctttgatgtgacgtctacgtatacgtacgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctgaaccggt

SEQ ID NO: 119 - DNA, 361 bp, synthetic construct
  source 1..361: mol_type = other DNA
ctagtgacgtcctgagcgacagtatagtgcacagtgactgcagcagtcattcctttgatgtacgcaactcctttgatgtctatgcgtcctttgatgttaaggattcctttgatgtaggtacatcctttgatgtccgtaaatcctttgatgtgacgtctacgtatacgtaagtggtgggggagtgaaaagagagatggagaaagaggggatgggcagaaagaggaggaggagtcaggggcagggcatggaggtgggtggggctgggctgccaaagcaggataaatgcacacctgcctgctggtctgggctccctgcctcgggctctcaccctcctctcctgcagctccagctttgtgctcta

SEQ ID NO: 120 - DNA, 421 bp, synthetic construct
  source 1..421: mol_type = other DNA
ctagtgacgtcctgagcgacagtatagtgcacagtgactgcagcagtcattcctttgatgtacgcaactcctttgatgtctatgcgtcctttgatgttaaggattcctttgatgtaggtacatcctttgatgtccgtaaatcctttgatgtgacgtctacgtatacgtaggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctcttta

SEQ ID NO: 121 - DNA, 895 bp, synthetic construct
  source 1..895: mol_type = other DNA
ggcctaactggccggtaccaccatggggaaggtggggtgatcacaggacagtcagcctcgcagaggacagagaccacccaggactgtcagggagaacatggacaggccctgagccgcagctcagccaacagacacggagagggagggtccccctggagccttccccaaggacagcagagcccagagtcacccacctccctccaccacagtcctctctttccaggacacacaagacacctccccctccacatgcaggatctggggactcctgagacctctgggcctgggtctccatccctgggtcagtggcggggttggtggtactggagacagagggctggtccctccccagccaccacccagtgagcctttttctagcccccagagccacctctgtcaccttcctgttgggcatcatcccaccttcccagagccctggagagcatggggagacccgggaccctgctgggtttctctgtcacaaaggaaaataatccccctggtgtgacagacccaaggacagaacacagcagaggtcagcactggggaagacaggttgtcctcccaggggatgggggtccatccaccttgccgaaaagatttgtctgaggaactgaaaatagaagggaaaaaagaggagggacaaaagaggcagaaatgagaggggaggggacagaggacacctgaataaagaccacacccatgacccacgtgatgctgagaagtactcctgccctaggaagagactcagggcagagggaggaaggacagcagaccagacagtcacagcagccttgacaaaacgttcctggaactaccggtgctagcctcgaggatatcaagatctggcctcggcggccaagcttggcaatccggtactgttggtaaagccacc

SEQ ID NO: 122 - DNA, 401 bp, synthetic construct
  source 1..401: mol_type = other DNA
ggcctaactggccggtaccttttgataaaaatcattaggtacggccgcggtgccagggcgtgcccttgggctccccgggcgcgaaactagtgacgtctacctgatcaaacatgcccggacatgtcgtaagacataaacatgcccggacatgtcctcgcaatctaacatgcccggacatgtcctcgcaatctaacatgcccggacatgtctgcaagctacaacatgcccggacatgtctacgtaacgcgtcccgacatgccccgcggcgcgccattaaccgccagatttgagtcgcgggacccgttggcagaggtgggaattcaccggtgctagcctcgaggatatcaagatctggcctcggcggccaagcttggcaatccggtactgttggtaaagccacc

SEQ ID NO: 123 - DNA, 560 bp, synthetic construct
  source 1..560: mol_type = other DNA
ggcctaactggccggtaccaactagtgacgtctacctgatcaaacatgcccggacatgtcgtaagacataaacatgcccggacatgtcctcgcaatctaacatgcccggacatgtcctcgcaatctaacatgcccggacatgtctgcaagctacaacatgcccggacatgtctacgtacatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgattagctagcctcgaggatatcaagatctggcctcggcggccaagcttggcaatccggtactgttggtaaagccacc

SEQ ID NO: 124 - DNA, 423 bp, synthetic construct
  source 1..423: mol_type = other DNA
ggcctaactggccggtaccaactagtgacgtctacctgatcaaacatgcccggacatgtcgtaagacataaacatgcccggacatgtcctcgcaatctaacatgcccggacatgtcctcgcaatctaacatgcccggacatgtctgcaagctacaacatgcccggacatgtctacgtacgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctgaaccggtgctagcctcgaggatatcaagatctggcctcggcggccaagcttggcaatccggtactgttggtaaagccacc

SEQ ID NO: 125 - DNA, 586 bp, synthetic construct
  source 1..586: mol_type = other DNA
ggcctaactggccggtaccaactagtgacgtctacctgatcaaacatgcccggacatgtcgtaagacataaacatgcccggacatgtcctcgcaatctaacatgcccggacatgtcctcgcaatctaacatgcccggacatgtctgcaagctacaacatgcccggacatgtctacccgttcgacaagcccggacatgctaagacataaacatgcccggacatgtcctcgcaatctaaccatgcccggacatgtcctcgcaatctaacatgcccggacatgtctgcaagctacaacatgcccggacatgtctacgtaagtggtgggggagtgaaaagagagatggagaaagaggggatgggcagaaagaggaggaggagtcaggggcagggcatggaggtgggtggggctgggctgccaaagcaggataaatgcacacctgcctgctggtctgggctccctgcctcgggctctcaccctcctctcctgcagctccagctttgtgctctaccggtgctagcctcgaggatatcaagatctggcctcggcggccaagcttggcaatccggtactgttggtaaagccacc

SEQ ID NO: 126 - DNA, 555 bp, synthetic construct
  source 1..555: mol_type = other DNA
ggcctaactggccggtaccttttgataaaaatcattaggtacggccgcggtgccagggcgtgcccttgggctccccgggcgcgaaactagtgacgtcctgagcgacagtatagtgcacagtgactgcagcagtcattcctttgatgtacgcaactcctttgatgtctatgcgtcctttgatgttaaggattcctttgatgtaggtacatcctttgatgtccgtaaatcctttgatgtgacgtctacgtatctacctgatcaaacatgcccggacatgtcgtaagacataaacatgcccggacatgtcctcgcaatctaacatgcccggacatgtcctcgcaatctaacatgcccggacatgtctgcaagctacaacatgcccggacatgtctacgtaacgcgtcccgacatgccccgcggcgcgccattaaccgccagatttgagtcgcgggacccgttggcagaggtgggaattcaccggtgctagcctcgaggatatcaagatctggcctcggcggccaagcttggcaatccggtactgttggtaaagccacc

SEQ ID NO: 127 - DNA, 869 bp, synthetic construct
  source 1..869: mol_type = other DNA
ggcctaactggccggtaccaactagtgacgtcctgagcgacagtatagtgcacagtgactgcagcagtcattcctttgatgtacgcaactcctttgatgtctatgcgtcctttgatgttaaggattcctttgatgtaggtacatcctttgatgtccgtaaatcctttgatgtgacgtctacgtatctacctgatcaaacatgcccggacatgtcgtaagacataaacatgcccggacatgtcctcgcaatctaacatgcccggacatgtcctcgcaatctaacatgcccggacatgtctgcaagctacaacatgcccggacatgtctacaatatacgtatctacctgatcaaacatgcccggacatgtcgtaagacataaacatgcccggacatgtcctcgcaatctaacatgcccggacatgtcctcgcaatctaacatgcccggacatgtctgcaagctacaacatgcccggacatgtctacgtacatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgattagctagcctcgaggatatcaagatctggcctcggcggccaagcttggcaatccggtactgttggtaaagccacc

SEQ ID NO: 128 - DNA, 13 bp, synthetic construct
  source 1..13: mol_type = other DNA
gttaattattaac

SEQ ID NO: 129 - DNA, 12 bp, synthetic construct
  source 1..12: mol_type = other DNA
tattttatcttt

SEQ ID NO: 130 - DNA, 526 bp, synthetic construct
  source 1..526: mol_type = other DNA
ggcctaactggccggtaccactagtaagcctcaagatttcctttaggctcttaggtaagaaatgtctaaggttcaaggaaaaaggttaagttggaagaatcccaggcaaaataagtgcgaatccacgacagttggtaacccggacccacattagaactcagaggtcaagcagaagcgaacgactggaattccagtcaggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctcttaccggtgctagcctcgaggatatcaagatctggcctcggcggccaagcttggcaatccggtactgttggtaaagccacc

SEQ ID NO: 131 - DNA, 13 bp, synthetic construct
  source 1..13: mol_type = other DNA
attccagatgttt

SEQ ID NO: 132 - DNA, 10 bp, synthetic construct
  source 1..10: mol_type = other DNA
accggaagtg

SEQ ID NO: 133 - DNA, 11 bp, synthetic construct
  source 1..11: mol_type = other DNA
ttctaatctat

SEQ ID NO: 134 - DNA, 10 bp, synthetic construct
  source 1..10: mol_type = other DNA
accggaaatg

SEQ ID NO: 135 - DNA, 11 bp, synthetic construct
  source 1..11: mol_type = other DNA
ggatgactcat

SEQ ID NO: 136 - DNA, 11 bp, synthetic construct
  source 1..11: mol_type = other DNA
ttcttggcaga

SEQ ID NO: 137 - DNA, 10 bp, synthetic construct
  source 1..10: mol_type = other DNA
accggaagcg

SEQ ID NO: 138 - DNA, 12 bp, synthetic construct
  source 1..12: mol_type = other DNA
gagaacaaagga

SEQ ID NO: 139 - DNA, 12 bp, synthetic construct
  source 1..12: mol_type = other DNA
taacttatcttt

SEQ ID NO: 140 - DNA, 11 bp, synthetic construct
  source 1..11: mol_type = other DNA
gggcgggaacg

SEQ ID NO: 141 - DNA, 11 bp, synthetic construct
  source 1..11: mol_type = other DNA
tcctttgatat

SEQ ID NO: 142 - DNA, 12 bp, synthetic construct
  source 1..12: mol_type = other DNA
tagcttatcttt

SEQ ID NO: 143 - DNA, 12 bp, synthetic construct
  source 1..12: mol_type = other DNA
aaacatcaaagg

SEQ ID NO: 144 - DNA, 14 bp, synthetic construct
  source 1..14: mol_type = other DNA
atgccacgtcacca

SEQ ID NO: 145 - DNA, 395 bp, synthetic construct
  source 1..395: mol_type = other DNA
ggcctaactggccggtaccactagtggggcggggtgatgacacagcaattcgggactttccacgcttgcgtgagaagagaccggaagtgaatgacacagcaattcgcttgcgtgagaagctgggactttcctaggggcggggttgggactttccacatgacacagcaatacactagtaacatttctctggcctaactggccggtaccgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctgaagaattcaccggtcgacgctagc

SEQ ID NO: 146 - DNA, 12 bp, synthetic construct
  source 1..12: mol_type = other DNA
ttcgcgctaaaa

SEQ ID NO: 147 - DNA, 348 bp, synthetic construct
  source 1..348: mol_type = other DNA
ggcctaactggccggtaccactagtgtcatctctttgaatattctgtagtttgaggagaatatttgttatattgcacaataaaataagtttgcaagttttttttttctgccccaaagagctctgtgtccttgaacataaaatacaaataaccgctatgctgttaattattaacaaatgtcccattttcaacctaaggaaataccataaagtaacagatataccaacaaaaggttaataattaacaggcattgcctgaaaagagtataaaaggctttcagcatgattttccatattgtgcttccaccactgccaataacaaaccggtgaattcaccggtcgacgctagc

SEQ ID NO: 148 - DNA, 196 bp, synthetic construct
  source 1..196: mol_type = other DNA
gaattcactagtgacagtatagtgcacagtgactgcagcagggtgactcatgatgccacgtcaccaggtgactcatgatgccacgtcaccaggtgactcatgatgccacgtcaccaggtgactcatgatgccacgtcaccaggtgactcatgggtacctataaaaggccagcagcagcctgaccacatctcatcca

SEQ ID NO: 149 - DNA, 196 bp, synthetic construct
  source 1..196: mol_type = other DNA
gaattcactagtagtatagtgcacagtgactgcagcagggtgactcatgatgatgccacgtcaccaatgccacgtcaccaggtgactcatgggtgactcatgatgccacgtcaccaatgccacgtcaccaggtgactcatgggtgactcatgggtacctataaaaggccagcagcagcctgaccacatctcatcca

SEQ ID NO: 150 - DNA, 196 bp, synthetic construct
  source 1..196: mol_type = other DNA
gaattcactagtctcaagtataaggtaagacatagttactgcgacatcggctagtaaaccggaagtgtctgtaaaccggaagtgatcgtaaaccggaagtgagcgtaaaccggaagtgctagtaaaccggaagtggaagtaaaccggaagtgggtacctataaaaggccagcagcagcctgaccacatctcatcca

SEQ ID NO: 151 - DNA, 202 bp, synthetic construct
  source 1..202: mol_type = other DNA
gaattcactagtgtactcaagtataaggtaagatttgcacacggtacgtactcatttgcacacggtacatgcgagtttgcacacggtacagctcagtttgcacacggtacgtcagcttttgcacacggtacatcagaatttgcacacggtacggtacctataaaaggccagcagcagcctgaccacatctcatccaccggtg

SEQ ID NO: 152 - DNA, 196 bp, synthetic construct
  source 1..196: mol_type = other DNA
gaattcactagttaattgctgagtcattgctgctatgtaattgctgagtcatatgcctatcctaattgctgagtcataatcgagatgtaattgctgagtcatgtccgacgcataattgctgagtcattctaactcgctaattgctgagtcatggtacctataaaaggccagcagcagcctgaccacatctcatcca

SEQ ID NO: 153 - DNA, 196 bp, synthetic construct
  source 1..196: mol_type = other DNA
gaattcactagtgctgagcgacagtatagtgcacagtgactgcagcagtcattatacgtaggggaatcccctcgaaggggaatcccctttaaggggaatcccctcgcaggggaatcccctctcaggggaatcccctaacaggggaatcccctggtacctataaaaggccagcagcagcctgaccacatctcatcca

SEQ ID NO: 154 - DNA, 196 bp, synthetic construct
  source 1..196: mol_type = other DNA
gaattcactagtgcatcctttgatgttacctgatcaaacatgcccggacatgtcgtaagacatatcctttgatgtctcgcaatctaacatgcccggacatgtcctcgcaatcttcctttgatgttgcaagctacaacatgcccggacatgtcggtacctataaaaggccagcagcagcctgaccacatctcatcca

SEQ ID NO: 155 - DNA, 196 bp, synthetic construct
  source 1..196: mol_type = other DNA
gaattcactagtgcaccattagtacttgatcagtatgccacgtcatcactactctatgccacgtcatctcctagatatgccacgtcatcgtaagactatgccacgtcatctacagcttatgccacgtcatcacgtacttatgccacgtcatcggtacctataaaaggccagcagcagcctgaccacatctcatcca

SEQ ID NO: 156 - DNA, 292 bp, synthetic construct
  source 1..292: mol_type = other DNA
ggcctaactggccggtaccactagtgtccccacccacacattcctgtccccacccacacattcctgtccccacccacacattcctgtccccacccacacattcctgtccccacccacacattcctgtccccacccacacattcctgtgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 157 - DNA, 282 bp, synthetic construct
  source 1..282: mol_type = other DNA
ggcctaactggccggtaccagcttgcatgcctgcaggtcggagtactgtcctccgagcggagtactgtcctccgagcggagtactgtcctccgagcggagtactgtcctccgagcggagtactgtcctccgagcggtgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 158 - DNA, 271 bp, synthetic construct
  source 1..271: mol_type = other DNA
ggcctaactggccggtaccactagtggttttgtggggttttgtggggttttgtggggttttgtggggttttgtggggttttgtggggttttgtggggttttgtggggttttgtggggttttgtggtgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 159 - DNA, 269 bp, synthetic construct
  source 1..269: mol_type = other DNA
ggcctaactggccggtaccactagtagccacttgaaattagccacttgaaattagccacttgaaattagccacttgaaattagccacttgaaattagccacttgaaattagccacttgaaatttgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 160 - DNA, 275 bp, synthetic construct
  source 1..275: mol_type = other DNA
ggcctaactggccggtaccactagtctgggaacaagtgctgggaacaagtgctgggaacaagtgctgggaacaagtgctgggaacaagtgctgggaacaagtgctgggaacaagtgctgggaacaagtgtgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 161 - DNA, 275 bp, synthetic construct
  source 1..275: mol_type = other DNA
ggcctaactggccggtaccactagtgactcctcaaggggactcctcaaggggactcctcaaggggactcctcaaggggactcctcaaggggactcctcaaggggactcctcaaggggactcctcaagggtgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 162 - DNA, 276 bp, synthetic construct
  source 1..276: mol_type = other DNA
ggcctaactggccggtaccactagtcgggctttgatctttcgggctttgatctttcgggctttgatctttcgggctttgatctttcgggctttgatctttcgggctttgatctttcgggctttgatcttttgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 163 - DNA, 273 bp, synthetic construct
  source 1..273: mol_type = other DNA
ggcctaactggccggtaccactagtgcgctttgatgtgcggggcggccctttgaagttggcgctttgatgtgcggggcggccctttgaagttggcgctttgatgtgcggggcggccctttgaagttgtgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 164 - DNA, 261 bp, synthetic construct
  source 1..261: mol_type = other DNA
ggcctaactggccggtaccactagtaacagctgttaacagctgttaacagctgttaacagctgttaacagctgttaacagctgttaacagctgttaacagctgttaacagctgtttgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 165 - DNA, 243 bp, synthetic construct
  source 1..243: mol_type = other DNA
ggcctaactggccggtaccactagtcacctgcacctgcacctgcacctgcacctgcacctgcacctgcacctgcacctgcacctgcacctgcacctgtgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 166 - DNA, 271 bp, synthetic construct
  source 1..271: mol_type = other DNA
ggcctaactggccggtaccactagtagttcaacacgtggtctgggagttcaacacgtggtctgggagttcaacacgtggtctgggagttcaacacgtggtctgggagttcaacacgtggtctgggtgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 167 - DNA, 275 bp, synthetic construct
  source 1..275: mol_type = other DNA
ggcctaactggccggtaccactagtgacagataagaaagacagataagaaagacagataagaaagacagataagaaagacagataagaaagacagataagaaagacagataagaaagacagataagaaatgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 168 - DNA, 270 bp, synthetic construct
  source 1..270: mol_type = other DNA
ggcctaactggccggtaccactagtttctaatctatttctaatctatttctaatctatttctaatctatttctaatctatttctaatctatttctaatctatttctaatctatttctaatctattgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 169 - DNA, 270 bp, synthetic construct
  source 1..270: mol_type = other DNA
ggcctaactggccggtaccactagtggtgactcatgggtgactcatgggtgactcatgggtgactcatgggtgactcatgggtgactcatgggtgactcatgggtgactcatgggtgactcatgtgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 170 - DNA, 270 bp, synthetic construct
  source 1..270: mol_type = other DNA
ggcctaactggccggtaccactagtcttctgggaaacttctgggaaacttctgggaaacttctgggaaacttctgggaaacttctgggaaacttctgggaaacttctgggaaacttctgggaaatgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 171 - DNA, 269 bp, synthetic construct
  source 1..269: mol_type = other DNA
ggcctaactggccggtaccactagtaattcttagaaataaattcttagaaataaattcttagaaataaattcttagaaataaattcttagaaataaattcttagaaataaattcttagaaatatgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 172 - DNA, 282 bp, synthetic construct
  source 1..282: mol_type = other DNA
ggcctaactggccggtaccactagtaaaacaaaggatcctttgttttaaaacaaaggatcctttgttttaaaacaaaggatcctttgttttaaaacaaaggatcctttgttttaaaacaaaggatcctttgttttctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 173 - DNA, 269 bp, synthetic construct
  source 1..269: mol_type = other DNA
ggcctaactggccggtaccactagtaaagtccaagtccaaaagtccaagtccaaaagtccaagtccaaaagtccaagtccaaaagtccaagtccaaaagtccaagtccaaaagtccaagtccatgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 174 - DNA, 272 bp, synthetic construct
  source 1..272: mol_type = other DNA
ggcctaactggccggtaccactagtggttttgtggagaggttttgtggtcgggttttgtgggacggttttgtggctaggttttgtggactggttttgtggtgcggttttgtgggtaggttttgtggtgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 175 - DNA, 270 bp, synthetic construct
  source 1..270: mol_type = other DNA
ggcctaactggccggtaccactagtagccacttgaaattagaagccacttgaaatttcgagccacttgaaattgacagccacttgaaattctaagccacttgaaattactagccacttgaaatttgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 176 - DNA, 280 bp, synthetic construct
  source 1..280: mol_type = other DNA
ggcctaactggccggtaccactagtctgggaacaagtgagactgggaacaagtgtcgctgggaacaagtggacctgggaacaagtgctactgggaacaagtgactctgggaacaagtgtgcctgggaacaagtgtgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 177 - DNA, 280 bp, synthetic construct
  source 1..280: mol_type = other DNA
ggcctaactggccggtaccactagtgactcctcaagggagagactcctcaagggtcggactcctcaaggggacgactcctcaagggctagactcctcaagggactgactcctcaagggtgcgactcctcaagggtgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 178 - DNA, 276 bp, synthetic construct
  source 1..276: mol_type = other DNA
ggcctaactggccggtaccactagtccggctttgatctttagacgggctttgatcttttcgcgggctttgatctttgaccgggctttgatctttctacgggctttgatctttactcgggctttgatcttttgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 179 - DNA, 279 bp, synthetic construct
  source 1..279: mol_type = other DNA
ggcctaactggccggtaccactagtgcgctttgatgtgcggggcggccctttgaagttgagagcgctttgatgtgcggggcggccctttgaagttgtcggcgctttgatgtgcggggcggccctttgaagttgtgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 180 - DNA, 272 bp, synthetic construct
  source 1..272: mol_type = other DNA
ggcctaactggccggtaccactagtaacagctgttagaaacagctgtttcgaacagctgttgacaacagctgttctaaacagctgttactaacagctgtttgcaacagctgttgtaaacagctgtttgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 181 - DNA, 258 bp, synthetic construct
  source 1..258: mol_type = other DNA
ggcctaactggccggtaccactagtcacctgagacacctgtcgcacctggaccacctgctacacctgactcacctgtgccacctgagacacctgtcgcacctggaccacctgtgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 182 - DNA, 283 bp, synthetic construct
  source 1..283: mol_type = other DNA
ggcctaactggccggtaccactagtagttcaacacgtggtctgggagaagttcaacacgtggtctgggtcgagttcaacacgtggtctggggacagttcaacacgtggtctgggctaagttcaacacgtggtctgggtgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 183 - DNA, 280 bp, synthetic construct
  source 1..280: mol_type = other DNA
ggcctaactggccggtaccactagtgacagataagaaaagagacagataagaaatcggacagataagaaagacgacagataagaaactagacagataagaaaactgacagataagaaatgcgacagataagaaatgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 184 - DNA, 266 bp, synthetic construct
  source 1..266: mol_type = other DNA
ggcctaactggccggtaccactagtttctaatctatagattctaatctattcgttctaatctatgacttctaatctatctattctaatctatactttctaatctattgcttctaatctattgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 185 - DNA, 267 bp, synthetic construct
  source 1..267: mol_type = other DNA
ggcctaactggccggtaccactagtggtgactcatgagaggtgactcatgtcgggtgactcatggacggtgactcatgctaggtgactcatgactggtgactcatgtgcggtgactcatgctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 186 - DNA, 266 bp, synthetic construct
  source 1..266: mol_type = other DNA
ggcctaactggccggtaccactagtcttctgggaaaagacttctgggaaatcgcttctgggaaagaccttctgggaaactacttctgggaaaactcttctgggaaatgccttctgggaaatgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 187 - DNA, 270 bp, synthetic construct
  source 1..270: mol_type = other DNA
ggcctaactggccggtaccactagtaattcttagaaataagaaattcttagaaatatcgaattcttagaaatagacaattcttagaaatactaaattcttagaaataactaattcttagaaatatgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 188 - DNA, 268 bp, synthetic construct
  source 1..268: mol_type = other DNA
ggcctaactggccggtaccactagtaaaacaaaggatcctttgttttagaaaaacaaaggatcctttgtttttcgaaaacaaaggatcctttgttttgacaaaacaaaggatcctttgtttttgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 189 - DNA, 270 bp, synthetic construct
  source 1..270: mol_type = other DNA
ggcctaactggccggtaccactagtaaagtccaagtccaagaaaagtccaagtccatcgaaagtccaagtccagacaaagtccaagtccactaaaagtccaagtccaactaaagtccaagtccatgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 190 - DNA, 339 bp, synthetic construct
  source 1..339: mol_type = other DNA
ggcctaactggccggtaccactagtaattcttagaaataaattcttagaaataaattcttagaaataaattcttagaaataaattcttagaaataaattcttagaaataaattcttagaaatatgcgctcccgacatgtcccgcggcgcgccattaaccgccagatttgagtcgcgggacccgttggcagaggtgggctagcctcgaggatatcaagatctggcctcggcggccaagcttggcaatccggtactgttggtaaagccaccatcctcgaggatatcaagatctggcctcggcggccaagcttggcaatccggtactgttggtaaagccacc

SEQ ID NO: 191 - DNA, 261 bp, synthetic construct
  source 1..261: mol_type = other DNA
ggcctaactggccggtaccactagtccaataaaaaccaataaaaaccaataaaaaccaataaaaaccaataaaaaccaataaaaaccaataaaaaccaataaaaaccaataaaaatgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 192 - DNA, 12 bp, synthetic construct
  source 1..12: mol_type = other DNA
gcgaccaccaaa

SEQ ID NO: 193 - DNA, 261 bp, synthetic construct
  source 1..261: mol_type = other DNA
ggcctaactggccggtaccactagttgtttacttatgtttacttatgtttacttatgtttacttatgtttacttatgtttacttatgtttacttatgtttacttatgtttacttatgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 194 - DNA, 267 bp, synthetic construct
  source 1..267: mol_type = other DNA
ggcctaactggccggtaccactagtaaaatggcgccattttaaaatggcgccattttaaaatggcgccattttaaaatggcgccattttaaaatggcgccattttaaaatggcgccatttttgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 195 - DNA, 259 bp, synthetic construct
  source 1..259: mol_type = other DNA
ggcctaactggccggtaccactagttattgtggttatattgtggttatattgtggttatattgtggttatattgtggttatattgtggttatattgtggttatattgtggttatgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 196 - DNA, 283 bp, synthetic construct
  source 1..283: mol_type = other DNA
ggcctaactggccggtaccactagtgaacaattgcagtgttgaacaattgcagtgttgaacaattgcagtgttgaacaattgcagtgttgaacaattgcagtgttgaacaattgcagtgttgaacaattgcagtgtttgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 197 - DNA, 271 bp, synthetic construct
  source 1..271: mol_type = other DNA
ggcctaactggccggtaccactagtccccaaaccaccccccccccccccaaaccaccccccccccccccaaaccaccccccccccccccaaaccaccccccccccccccaaaccacccccccccctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 198 - DNA, 243 bp, synthetic construct
  source 1..243: mol_type = other DNA
cactagtaccggaagtaaccggaagtaaccggaagtaaccggaagtaaccggaagtaaccggaagtaaccggaagtaaccggaagtaaccggaagtatgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 199 - DNA, 261 bp, synthetic construct
  source 1..261: mol_type = other DNA
ggcctaactggccggtaccactagtggcacgtgttggcacgtgttggcacgtgttggcacgtgttggcacgtgttggcacgtgttggcacgtgttggcacgtgttggcacgtgtttgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 200 - DNA, 262 bp, synthetic construct
  source 1..262: mol_type = other DNA
ggcctaactggccggtaccactagtcgagcagctggtgcgagcagctggtgcgagcagctggtgcgagcagctggtgcgagcagctggtgcgagcagctggtgcgagcagctggtgtgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 201 - DNA, 251 bp, synthetic construct
  source 1..251: mol_type = other DNA
ggcctaactggccggtaccactagttccagatgtttccagatgtttccagatgtttccagatgtttccagatgtttccagatgtttccagatgtttccagatgtttgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 202 - DNA, 251 bp, synthetic construct
  source 1..251: mol_type = other DNA
ggcctaactggccggtaccactagtatagtaaacaatagtaaacaatagtaaacaatagtaaacaatagtaaacaatagtaaacaatagtaaacaatagtaaacatgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 203 - DNA, 248 bp, synthetic construct
  source 1..248: mol_type = other DNA
ggcctaactggccggtaccactagttaatccctaatccctaatccctaatccctaatccctaatccctaatccctaatccctaatccctaatccctaatccctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 204 - DNA, 251 bp, synthetic construct
  source 1..251: mol_type = other DNA
ggcctaactggccggtaccactagtctaattaactaattaactaattaactaattaactaattaactaattaactaattaactaattaactaattaactaattaatgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 205 - DNA, 251 bp, synthetic construct
  source 1..251: mol_type = other DNA
ggcctaactggccggtaccactagtcccaattagccccaattagccccaattagccccaattagccccaattagccccaattagccccaattagccccaattagctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 206 - DNA, 243 bp, synthetic construct
  source 1..243: mol_type = other DNA
ggcctaactggccggtaccactagtcaattacaattacaattacaattacaattacaattacaattacaattacaattacaattacaattacaattatgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 207 - DNA, 267 bp, synthetic construct
  source 1..267: mol_type = other DNA
ggcctaactggccggtaccactagtaaaaccggttttaaaaccggttttaaaaccggttttaaaaccggttttaaaaccggttttaaaaccggttttaaaaccggttttaaaaccggtttttgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 208 - DNA, 259 bp, synthetic construct
  source 1..259: mol_type = other DNA
ggcctaactggccggtaccactagttgtttacttaagatgtttacttatcgtgtttacttagactgtttacttactatgtttacttaacttgtttacttatgctgtttacttatgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 209 - DNA, 282 bp, synthetic construct
  source 1..282: mol_type = other DNA
ggcctaactggccggtaccactagtaaaatggcgccatttttcgaaaatggcgccattttgacaaaatggcgccattttctaaaaatggcgccattttactaaaatggcgccatttttgcaaaatggcgccatttttgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 210 - DNA, 252 bp, synthetic construct
  source 1..252: mol_type = other DNA
ggcctaactggccggtaccactagttattgtggttatcgtattgtggttagactattgtggttactatattgtggttaacttattgtggttatgctattgtggttatgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 211 - DNA, 263 bp, synthetic construct
  source 1..263: mol_type = other DNA
ggcctaactggccggtaccactagtgaacaattgcagtgttgacgaacaattgcagtgttctagaacaattgcagtgttactgaacaattgcagtgtttgcgaacaattgcagtgtttgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 212 - DNA, 260 bp, synthetic construct
  source 1..260: mol_type = other DNA
ggcctaactggccggtaccactagtccccaaaccaccccccccccgacccccaaaccaccccccccccctaccccaaaccaccccccccccactccccaaaccacccccccccctgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 213 - DNA, 259 bp, synthetic construct
  source 1..259: mol_type = other DNA
ggcctaactggccggtaccactagtaccggaagtaagaaccggaagtatcgaccggaagtagacaccggaagtactaaccggaagtaactaccggaagtatgcaccggaagtatgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 214 - DNA, 259 bp, synthetic construct
  source 1..259: mol_type = other DNA
ggcctaactggccggtaccactagtggcacgtgttagaggcacgtgtttcgggcacgtgttgacggcacgtgttctaggcacgtgttactggcacgtgtttgcggcacgtgtttgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 215 - DNA, 264 bp, synthetic construct
  source 1..264: mol_type = other DNA
ggcctaactggccggtaccactagtcgagcagctggtgagacgagcagctggtgtcgcgagcagctggtggaccgagcagctggtgctacgagcagctggtgactcgagcagctggtgtgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 216 - DNA, 259 bp, synthetic construct
  source 1..259: mol_type = other DNA
ggcctaactggccggtaccactagttccagatgttagatccagatgtttcgtccagatgttgactccagatgttctatccagatgttacttccagatgtttgctccagatgtttgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 217 - DNA, 259 bp, synthetic construct
  source 1..259: mol_type = other DNA
ggcctaactggccggtaccactagtatagtaaacaagaatagtaaacatcgatagtaaacagacatagtaaacactaatagtaaacaactatagtaaacatgcatagtaaacatgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 218 - DNA, 258 bp, synthetic construct
  source 1..258: mol_type = other DNA
ggcctaactggccggtaccactagttaatcccagataatccctcgtaatcccgactaatcccctataatcccacttaatccctgctaatcccacttaatccctgctaatccctgcgctcccgacatgccccgcggcgcgtcattaaccgccagatttgagtcgcgggacccgttggcagaggtgggctagcctcgaggatatcaagatctggcctcggcggccaagcttggcaatccggtactgttggtaaagccacc

SEQ ID NO: 219 - DNA, 256 bp, synthetic construct
  source 1..256: mol_type = other DNA
ggcctaactggccggtaccactagtctaattaaagactaattaatcgctaattaagacctaattaactactaattaaactctaattaatgcctaattaaactctaattaatgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 220 - DNA, 259 bp, synthetic construct
  source 1..259: mol_type = other DNA
ggcctaactggccggtaccactagtcccaattagcagacccaattagctcgcccaattagcgaccccaattagcctacccaattagcactcccaattagctgccccaattagctgcgctcccgacatgccctgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 221 - DNA, 258 bp, synthetic construct
  source 1..258: mol_type = other DNA
ggcctaactggccggtaccactagtcaattaagacaattatcgcaattagaccaattactacaattaactcaattatgccaattaactcaattatgccaattaagacaattatgcgctcccgacatgccccgcggcgtgccattaaccgccagatttgagtcgcgggacccgttggcagaggtgggctagcctcgaggatatcaagatctggcctcggcggccaagcttggcaatccggtactgttggtaaagccacc

SEQ ID NO: 222 - DNA, 273 bp, synthetic construct
  source 1..273: mol_type = other DNA
ggcctaactggccggtaccactagtaaaaccggttttagaaaaaccggtttttcgaaaaccggttttgacaaaaccggttttctaaaaaccggttttactaaaaccggtttttgcaaaaccggtttttgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 223 - DNA, 226 bp, synthetic construct
  source 1..226: mol_type = other DNA
ggcctaactggccggtaccactagtggtgactcatgggtgactcatgggtgactcatgggtgactcatgggtgactcatgtgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 224 - DNA, 292 bp, synthetic construct
  source 1..292: mol_type = other DNA
ggcctaactggccggtaccactagtggtgactcatgggtgactcatgggtgactcatgggtgactcatgggtgactcatgggtgactcatgggtgactcatgggtgactcatgggtgactcatgggtgactcatgggtgactcatgtgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 225 - DNA, 248 bp, synthetic construct
  source 1..248: mol_type = other DNA
ggcctaactggccggtaccactagtggtgactcatgggtgactcatgggtgactcatgggtgactcatgggtgactcatgggtgactcatgggtgactcatgtgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 226 - DNA, 250 bp, synthetic construct
  source 1..250: mol_type = other DNA
ggcctaactggccggtaccactagtggtgactcatgggtgactcatgggtgactcatgggtgactcatgggtgactcatgggtgactcatgggtgactcatgggtgactcatgggtgactcatgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 227 - DNA, 240 bp, synthetic construct
  source 1..240: mol_type = other DNA
ggcctaactggccggtaccactagtggtgactcatgggtgactcatgggtgactcatgggtgactcatgggtgactcatgggtgactcatgggtgactcatgggtgactcatgggtgactcatgcggtgctagctataaaaggccagcagcagcctgaccacatctcatcctcctcgaggatatcaagatctggcctcggcggccaagcttggcaatccggtactgttggtaaagccacc

SEQ ID NO: 228 - DNA, 230 bp, synthetic construct
  source 1..230: mol_type = other DNA
ggcctaactggccggtaccactagtggtgactcatgggtgactcatgggtgactcatgggtgactcatgggtgactcatgggtgactcatgggtgactcatgggtgactcatgggtgactcatgtataaaaggccagcagcagcctgaccacatctcatcctcctcgaggatatcaagatctggcctcggcggccaagcttggcaatccggtactgttggtaaagccacc

SEQ ID NO: 229 - DNA, 255 bp, synthetic construct
  source 1..255: mol_type = other DNA
ggcctaactggccggtaccactagtggtgactcatgggtgactcatgggtgactcatgggtgactcatgggtgactcatgggtgactcatgggtgactcatgggtgactcatgggtgactcatgacatctttcagggaccggtgctagctataaaaggccagcagcagcctgaccacatctcatcctcctcgaggatatcaagatctggcctcggcggccaagcttggcaatccggtactgttggtaaagccacc

SEQ ID NO: 230 - DNA, 280 bp, synthetic construct
  source 1..280: mol_type = other DNA
ggcctaactggccggtaccactagtggtgactcatgggtgactcatgggtgactcatgggtgactcatgggtgactcatgggtgactcatgggtgactcatgggtgactcatgggtgactcatgtggctattagcagtaccgcttagacacatctttcagggaccggtgctagctataaaaggccagcagcagcctgaccacatctcatcctcctcgaggatatcaagatctggcctcggcggccaagcttggcaatccggtactgttggtaaagccacc

SEQ ID NO: 231 - DNA, 310 bp, synthetic construct
  source 1..310: mol_type = other DNA
ggcctaactggccggtaccactagtctgtttacctgtttacctgtttacctgtttacctgtttacggtgactcatgggtgactcatgggtgactcatgggtgactcatgggtgactcatgggtgactcatgggtgactcatgggtgactcatgggtgactcatgtgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 232 - DNA, 322 bp, synthetic construct
  source 1..322: mol_type = other DNA
ggcctaactggccggtaccactagtctgtttacagactgtttactcgctgtttacgacctgtttacctactgtttacggtgactcatgggtgactcatgggtgactcatgggtgactcatgggtgactcatgggtgactcatgggtgactcatgggtgactcatgggtgactcatgtgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 233 - DNA, 286 bp, synthetic construct
  source 1..286: mol_type = other DNA
ggcctaactggccggtaccactagtggtgactcatgggtgactcatgggtgactcatgggtgactcatgggtgactcatgggtgactcatgggtgactcatgggtgactcatgggtgactcatgcataggcctctgaacaacgcgtcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 234 - DNA, 306 bp, synthetic construct
  source 1..306: mol_type = other DNA
ggcctaactggccggtaccactagtggtgactcatgggtgactcatgggtgactcatgggtgactcatgggtgactcatgggtgactcatgggtgactcatgggtgactcatgggtgactcatgcataggcctctgatagagctgcgatagaccaagacaacgcgtcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 235 - DNA, 364 bp, synthetic construct
  source 1..364: mol_type = other DNA
ggcctaactggccggtaccactagtggtgactcatgggtgactcatgggtgactcatgggtgactcatgggtgactcatgggtgactcatgggtgactcatgggtgactcatgggtgactcatgcatagaaacgacgcaatatctccatagggttaacggcggaacttgacggcgtccattagccacttggtcatgggacagggggggaaaacggacaacgcgtcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 236 - DNA, 306 bp, synthetic construct
  source 1..306: mol_type = other DNA
ggcctaactggccggtaccactagtggtgactcatgggtgactcatgggtgactcatgggtgactcatgggtgactcatgggtgactcatgggtgactcatgggtgactcatgggtgactcatgcataccggaagtacttgcgcaatgaccggaagtacaacgcgtcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 237 - DNA, 364 bp, synthetic construct
  source 1..364: mol_type = other DNA
ggcctaactggccggtaccactagtggtgactcatgggtgactcatgggtgactcatgggtgactcatgggtgactcatgggtgactcatgggtgactcatgggtgactcatgggtgactcatgcatttgcgcaacaggggcggggtgatgacacagcaattcgcttgcgtgagaagagaccggaagtgagggactttccacatgacacagcaatacaacgcgtcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 238 - DNA, 420 bp, synthetic construct
  source 1..420: mol_type = other DNA
ggcctaactggccggtaccactagtggtgactcatgggtgactcatgggtgactcatgggtgactcatgggtgactcatgggtgactcatgggtgactcatgggtgactcatgggtgactcatgcatggggcggggtgatgacacagcaattcgggactttccacgcttgcgtgagaagagaccggaagtgaatgacacagcaattcgcttgcgtgagaagctgggactttcctaggggcggggttgggactttccacatgacacagcaatacaacgcgtcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 239 - DNA, 204 bp, synthetic construct
  source 1..204: mol_type = other DNA
ggcctaactggccggtaccactagtaccggaagtacttgcgcaatgaccggaagtacaacgcgtcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 240 - DNA, 262 bp, synthetic construct
  source 1..262: mol_type = other DNA
ggcctaactggccggtaccactagtttgcgcaacaggggcggggtgatgacacagcaattcgcttgcgtgagaagagaccggaagtgagggactttccacatgacacagcaatacaacgcgtcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 241 - DNA, 318 bp, synthetic construct
  source 1..318: mol_type = other DNA
ggcctaactggccggtaccactagtggggcggggtgatgacacagcaattcgggactttccacgcttgcgtgagaagagaccggaagtgaatgacacagcaattcgcttgcgtgagaagctgggactttcctaggggcggggttgggactttccacatgacacagcaatacaacgcgtcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 242 - DNA, 307 bp, synthetic construct
  source 1..307: mol_type = other DNA
ggcctaactggccggtaccactagtggtgactcatgggtgactcatgggtgactcatgggtgactcatgggtgactcatgggtgactcatgggtgactcatgggtgactcatgggtgactcatgcatacaacgcgtcccgacatgccccgacatgcccatcgacatgccccgacatgcccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 243 - DNA, 431 bp, synthetic construct
  source 1..431: mol_type = other DNA
ggcctaactggccggtaccactagtggtgactcatgggtgactcatgggtgactcatgggtgactcatgggtgactcatgggtgactcatgggtgactcatgggtgactcatgggtgactcatgcatgaattcggacatgcccgggcatgtccccagggacatgcccgggcatgtccccagagacatgtccagacatgtccccaggaacatgtcccaacatgttgtccaggagacatgtccagacatgtccccaggaacatgtcccaacatgttgtactagtacaacgcgtcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 244 - DNA, 338 bp, synthetic construct
  source 1..338: mol_type = other DNA
ggcctaactggccggtacctgccactcaaagtggcacactccctgctcaggaggccgggagggaggacacagccctggcaactcctctgccccggggggtcaggaaggggtcaccccacactccagaaccctacagaatgtggccttggcttttcccatcaagagctggggaaagccaggccccgacttcattaccccctgcccccgtcccatgctcagtgggccccatcgtgggtccatgccacactcccaactgagcagccccgcagccccgcgtgtcacagacatggggcctcctaattgctgctgaggtcccaatccctggctggacgtgcctg

SEQ ID NO: 245 - DNA, 386 bp, synthetic construct
  source 1..386: mol_type = other DNA
ggcctaactggccggtaccactagtggtgactcatgggtgactcatgggtgactcatgggtgactcatgggtgactcatgggtgactcatgggtgactcatgggtgactcatgactagtgtccccacccacacattcctgtccccacccacacattcctgtccccacccacacattcctgtccccacccacacattcctgtccccacccacacattcctgtccccacccacacattcctgtgcgctcccgacatgccccgcggcgcgccattaaccgccagatttgagtcgcgggacccgttggcagaggtgggctagcctcgaggatatcaagatctggcctcggcggccaagcttggcaatccggtactgttggtaaagccacc

SEQ ID NO: 246 - DNA, 366 bp, synthetic construct
  source 1..366: mol_type = other DNA
ggcctaactggccggtaccaagacaggttgtcctcccaggggatgggggtccatccaccttgccgaaaagatttgtctgaggaactgaaaatagaagggaaaaaagaggagggacaaaagaggcagaaatgagaggggaggggacagaggacacctgaataaagaccacacccatgacccacgtgatgctgagaagtactcctgccctaggaagagactcagggcagagggaggaaggacagcagaccagacagtcacagcagccttgacaaaacgttcctggaactaccggtgctagcctcgaggatatcaagatctggcctcggcggccaagcttggcaatccggtactgttggtaaagccacc

SEQ ID NO: 247 - DNA, 212 bp, synthetic construct
  source 1..212: mol_type = other DNA
ggcctaactggccggtaccatgacccacgtgatgctgagaagtactcctgccctaggaagagactcagggcagagggaggaaggacagcagaccagacagtcacagcagccttgacaaaacgttcctggaactaccggtgctagcctcgaggatatcaagatctggcctcggcggccaagcttggcaatccggtactgttggtaaagccacc

SEQ ID NO: 248 - DNA, 868 bp, synthetic construct
  source 1..868: mol_type = other DNA
ggcctaactggccggtaccctggatgctcatcccgccaccgtcgcccaccccgccgctgcagaaaggcagcaactgccacacacctaagcaacttggcgggctattcgccctgcagctgccgccagcgcgcggctcccgccagcgcgctggcaatcaaaagtcggagaaagcgcgaaacctccaggcacctcccactccgcccagctaccgcgcagctcctccctagcctccactgggagacaggggacgcccatgagcgggaaagagcagggcggtgattgcttagtttatcctgggacacgggaactggccgtggactgagtggtgccggggaggggatcactgagaccgggaagggtcatccagacaaatagggagggtgggcgggttggcgcgcagtaccctcggcccggccttcagacccacctgcgcgcgctgcgcgctcatccggtccttcccttcaatcactgtctggagtgatgataattggcttccacagtggatgagagatgagtcatttacatccaatgagagaaaaacagcctccagagactcttcgtccattggccagcgagagtgtcagttcccaggctcctgccgcgcacgggcgagcccttctaggcgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctgaaccggtgctagcctcgaggatatcaagatctggcctcggcggccaagcttggcaatccggtactgttggtaaagccacc

SEQ ID NO: 249 - DNA, 543 bp, synthetic construct
  source 1..543: mol_type = other DNA
ggcctaactggccggtacctgagaccgggaagggtcatccagacaaatagggagggtgggcgggttggcgcgcagtaccctcggcccggccttcagacccacctgcgcgcgctgcgcgctcatccggtccttcccttcaatcactgtctggagtgatgataattggcttccacagtggatgagagatgagtcatttacatccaatgagagaaaaacagcctccagagactcttcgtccattggccagcgagagtgtcagttcccaggctcctgccgcgcacgggcgagcccttctaggcgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctgaaccggtgctagcctcgaggatatcaagatctggcctcggcggccaagcttggcaatccggtactgttggtaaagccacc

SEQ ID NO: 250 - DNA, 263 bp, synthetic construct
  source 1..263: mol_type = other DNA
ggcctaactggccggtaccgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctgaaccggtgctagcctcgaggatatcaagatctggcctcggcggccaagcttggcaatccggtactgttggtaaagccacc

SEQ ID NO: 251 - DNA, 242 bp, synthetic construct
  source 1..242: mol_type = other DNA
ggcctaactggccggtaccctgctcctccttcttgcgggccgcgccctgccggcagtgacgtgccccgccctgcagccgcgggattcaaactcccggaagcggcatccacacctgatggtgtgactcggccgacgcgagcgccgcgcttcgcttcagctgctaaccggtgctagcctcgaggatatcaagatctggcctcggcggccaagcttggcaatccggtactgttggtaaagccacc

SEQ ID NO: 252 - DNA, 348 bp, synthetic construct
  source 1..348: mol_type = other DNA
ggcctaactggccggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctcttaccggtgctagcctcgaggatatcaagatctggcctcggcggccaagcttggcaatccggtactgttggtaaagccacc

SEQ ID NO: 253 - DNA, 710 bp, synthetic construct
  source 1..710: mol_type = other DNA
ggcctaactggccggtaccttgttttgacaggagcagggaagtattgtagaaaataatttttatcataatggagtatggcaggttatatgactgcgaggatcagaattgtgaatcatctcttgtgtgtcttcaagtaaataaaggcaatctgcccacggagcagaaaaaaaatctacaaactacaaactctgtccaatcatgtaaagacaaatcagccttcaggcaaatcaaatgtcttcattcaaagtctacctggatttggcactctgcccatcgtttcaaaacctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgattaccggtgctagcctcgaggatatcaagatctggcctcggcggccaagcttggcaatccggtactgttggtaaagccacc

SEQ ID NO: 254 - DNA, 445 bp, synthetic construct
  source 1..445: mol_type = other DNA
ggcctaactggccggtaccacctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgattaccggtgctagcctcgaggatatcaagatctggcctcggcggccaagcttggcaatccggtactgttggtaaagccacc

SEQ ID NO: 255 - DNA, 275 bp, synthetic construct
  source 1..275: mol_type = other DNA
ggcctaactggccggtacccagtgggtaggtctagcagtggcgcagcaatagagcgctccggagcgtctcattggctggatcaaacccaagcgagccattgattggtcgacgcccccagagggttacaattcaaacgcgggcgggcgggcccgcagtcctgcagttgcagtcgtgttctccgagttcctgtctctctgccgagctagcctcgaggatatcaagatctggcctcggcggccaagcttggcaatccggtactgttggtaaagccacc

SEQ ID NO: 256 - DNA, 289 bp, synthetic construct
  source 1..289: mol_type = other DNA
ggcctaactggccggtaccagtggtgggggagtgaaaagagagatggagaaagaggggatgggcagaaagaggaggaggagtcaggggcagggcatggaggtgggtggggctgggctgccaaagcaggataaatgcacacctgcctgctggtctgggctccctgcctcgggctctcaccctcctctcctgcagctccagctttgtgctctaccggtgctagcctcgaggatatcaagatctggcctcggcggccaagcttggcaatccggtactgttggtaaagccacc

SEQ ID NO: 257 - DNA, 557 bp, synthetic construct
  source 1..557: mol_type = other DNA
ggcctaactggccggtaccactagtcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgcttcccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcgatgccgcgcgctcctagctatcctcgaggatatcaagatctggcctcggcggccaagcttggcaatccggtactgttggtaaagccacc

SEQ ID NO: 258 - DNA, 971 bp, synthetic construct
  source 1..971: mol_type = other DNA
ggcctaactggccggtaccctggcaggaagcctactgagatttattgaaaaggaaaccgaattatcagggcactcgtttgcaacgccaacctgggctgtgttcggggcatgcccagcctgctgtctgcagtgtgaagctctttagaagccactgcaaccacaggccgcccgacaggaacagagacactgaaaacgggcccgcagcaaggcaggctcagcagccaacagtcacacccaggaagcagtatttttcttctgctcctggactctcttgcggtgtatggctgcttccctttggtctgagccaggccgatggtctcagaaatagacacccattgactttcttttccagcgctgggacatacagaccccgcctccatcccagggtgtctataggaaggatggcggctgctgcagggaggagggtctcctgtcttcctaagggcgcccctccaccagcctgtgggtgggtccgaggcacttccattccgatatctagctggccaaatcctgcaaaccttgaggcaggaagaacctgcagagcacatgggacttgcagcggacatgctttaaagaggtgccccaggcccgtccaccgccctcggccaccctccgtgtcctctggggagcagctgcggaagattcgagtcagaatagcaagaaggaaccgcagcagaaggtacaactcccagcatgccctgcgcccgccacgcccacaaggccaggcgcagatgggcgtggggcgggactttcccggctcgcctcgcgccgtccactcccagaaggcagcgggcgagggcgtggggccggggctctcccggcatgctctgcggcgcgcctccgcccgcgcgatttgaatcctgcgtttgagtcgtcttggcggaggttgtggtgacgcgctagcctcgaggatatcaagatctggcctcggcggccaagcttggcaatccggtactgttggtaaagccacc

SEQ ID NO: 259 - DNA, 225 bp, synthetic construct
  source 1..225: mol_type = other DNA
ggcctaactggccggtaccactcccagaaggcagcgggcgagggcgtggggccggggctctcccggcatgctctgcggcgcgcctccgcccgcgcgatttgaatcctgcgtttgagtcgtcttggcggaggttgtggtgacgcgctagctattctagcctcgaggatatcaagatctggcctcggcggccaagcttggcaatccggtactgttggtaaagccacc

SEQ ID NO: 260 - DNA, 355 bp, synthetic construct
  source 1..355: mol_type = other DNA
ggcctaactggccggtaccactagtggtgactcatgggtgactcatgggtgactcatgggtgactcatgggtgactcatgggtgactcatgggtgactcatgggtgactcatgggtgactcatggtgatcatgctagcctcgaggatatcaagatcggtaccatgacccacgtgatgctgagaagtactcctgccctaggaagagactcagggcagagggaggaaggacagcagaccagacagtcacagcagccttgacaaaacgttcctggaactaccggtgctagcctcgaggatatcaagatctggcctcggcggccaagcttggcaatccggtactgttggtaaagccacc

SEQ ID NO: 261 - DNA, 377 bp, synthetic construct
  source 1..377: mol_type = other DNA
ggcctaactggccggtaccactagtggtgactcatgggtgactcatgggtgactcatgggtgactcatgggtgactcatgggtgactcatgggtgactcatgggtgactcatgggtgactcatggtgatcatcgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctgaaccggtgctagcctcgaggatatcaagatctggcctcggcggccaagcttggcaatccggtactgttggtaaagccacc

SEQ ID NO: 262 - DNA, 491 bp, synthetic construct
  source 1..491: mol_type = other DNA
ggcctaactggccggtaccactagtggtgactcatgggtgactcatgggtgactcatgggtgactcatgggtgactcatgggtgactcatgggtgactcatgggtgactcatgggtgactcatggtgatcatgctagcctcgaggatatcaagatcggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctcttaccggtgctagcctcgaggatatcaagatctggcctcggcggccaagcttggcaatccggtactgttggtaaagccacc

SEQ ID NO: 263 - DNA, 432 bp, synthetic construct
  source 1..432: mol_type = other DNA
ggcctaactggccggtaccactagtggtgactcatgggtgactcatgggtgactcatgggtgactcatgggtgactcatgggtgactcatgggtgactcatgggtgactcatgggtgactcatggtgatcatgctagcctcgaggatatcaagatcggtaccagtggtgggggagtgaaaagagagatggagaaagaggggatgggcagaaagaggaggaggagtcaggggcagggcatggaggtgggtggggctgggctgccaaagcaggataaatgcacacctgcctgctggtctgggctccctgcctcgggctctcaccctcctctcctgcagctccagctttgtgctctaccggtgctagcctcgaggatatcaagatctggcctcggcggccaagcttggcaatccggtactgttggtaaagccacc

SEQ ID NO: 264 - DNA, 375 bp, synthetic construct
  source 1..375: mol_type = other DNA
ggcctaactggccggtaccactagtgtccccacccacacattcctgtccccacccacacattcctgtccccacccacacattcctgtccccacccacacattcctgtccccacccacacattcctgtccccacccacacattcctgaccggtgctagcctcgaggatatcaagatcggtaccatgacccacgtgatgctgagaagtactcctgccctaggaagagactcagggcagagggaggaaggacagcagaccagacagtcacagcagccttgacaaaacgttcctggaactaccggtgctagcctcgaggatatcaagatctggcctcggcggccaagcttggcaatccggtactgttggtaaagccacc

SEQ ID NO: 265 - DNA, 391 bp, synthetic construct
  source 1..391: mol_type = other DNA
ggcctaactggccggtaccactagtgtccccacccacacattcctgtccccacccacacattcctgtccccacccacacattcctgtccccacccacacattcctgtccccacccacacattcctgtccccacccacacattcctgcgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctgaaccggtgctagcctcgaggatatcaagatctggcctcggcggccaagcttggcaatccggtactgttggtaaagccacc

SEQ ID NO: 266 - DNA, 476 bp, synthetic construct
  source 1..476: mol_type = other DNA
ggcctaactggccggtaccactagtgtccccacccacacattcctgtccccacccacacattcctgtccccacccacacattcctgtccccacccacacattcctgtccccacccacacattcctgtccccacccacacattcctgcggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctcttaccggtgctagcctcgaggatatcaagatctggcctcggcggccaagcttggcaatccggtactgttggtaaagccacc

SEQ ID NO: 267 - DNA, 608 bp, synthetic construct
  source 1..608: mol_type = other DNA
ggcctaactggccggtaccactagtgtccccacccacacattcctgtccccacccacacattcctgtccccacccacacattcctgtccccacccacacattcctgtccccacccacacattcctgtccccacccacacattcctgaccggtgctagcctcgaggatatcaagatcggtaccacctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgattaccggtgctagcctcgaggatatcaagatctggcctcggcggccaagcttggcaatccggtactgttggtaaagccacc

SEQ ID NO: 268 - DNA, 452 bp, synthetic construct
  source 1..452: mol_type = other DNA
ggcctaactggccggtaccactagtgtccccacccacacattcctgtccccacccacacattcctgtccccacccacacattcctgtccccacccacacattcctgtccccacccacacattcctgtccccacccacacattcctgaccggtgctagcctcgaggatatcaagatcggtaccagtggtgggggagtgaaaagagagatggagaaagaggggatgggcagaaagaggaggaggagtcaggggcagggcatggaggtgggtggggctgggctgccaaagcaggataaatgcacacctgcctgctggtctgggctccctgcctcgggctctcaccctcctctcctgcagctccagctttgtgctctaccggtgctagcctcgaggatatcaagatctggcctcggcggccaagcttggcaatccggtactgttggtaaagccacc

SEQ ID NO: 269 - DNA, 588 bp, synthetic construct
  source 1..588: mol_type = other DNA
ggcctaactggccggtaccactagtggtgactcatgggtgactcatgggtgactcatgggtgactcatgggtgactcatgggtgactcatgggtgactcatgggtgactcatgggtgactcatggtgatcatgctagcctcgaggatatcaagatcggtaccacctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgattaccggtgctagcctcgaggatatcaagatctggcctcggcggccaagcttggcaatccggtactgttggtaaagccacc

SEQ ID NO: 270 - DNA, 12 bp, synthetic construct
  source 1..12: mol_type = other DNA
gcaaccaccgaa

SEQ ID NO: 271 - DNA, 520 bp, synthetic construct
  source 1..520: mol_type = other DNA
aattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgactagtggtgactcatgggtgactcatgggtgactcatgggtgactcatgggtgactcatgtgcgctcccgacatgccccgcggcgcgccattaaccgccagatttgagtcgcgggacccgttggcagaggtgggaattcaccggtcgacgctagc

SEQ ID NO: 272 - DNA, 11 bp, synthetic construct
  source 1..11: mol_type = other DNA
tcctttgaact

SEQ ID NO: 273 - DNA, 542 bp, synthetic construct
  source 1..542: mol_type = other DNA
aattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgactagtggtgactcatgggtgactcatgggtgactcatgggtgactcatgggtgactcatgggtgactcatgggtgactcatgtgcgctcccgacatgccccgcggcgcgccattaaccgccagatttgagtcgcgggacccgttggcagaggtgggaattcaccggtcgacgctagc

SEQ ID NO: 274 - DNA, 294 bp, synthetic construct
  source 1..294: mol_type = other DNA
tctgtagtttgaggagaatatttgttatattgcacaataaaataagtttgcaagttttttttttctgccccaaagagctctgtgtccttgaacataaaatacaaataaccgctatgctgttaattattaacaaatgtcccattttcaacctaaggaaataccataaagtaacagatataccaacaaaaggttaataattaacaggcattgcctgaaaagagtataaaaggctttcagcatgattttccatattgtgcttccaccactgccaataacaaaccggtcgacgctagc

SEQ ID NO: 275 - DNA, 10 bp, synthetic construct
  source 1..10: mol_type = other DNA
gtcgttaaat

SEQ ID NO: 276 - DNA, 10 bp, synthetic construct
  source 1..10: mol_type = other DNA
agaggaagtg

SEQ ID NO: 277 - DNA, 11 bp, synthetic construct
  source 1..11: mol_type = other DNA
tacttggcaga

SEQ ID NO: 278 - DNA, 1196 bp, synthetic construct
  source 1..1196: mol_type = other DNA
gcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcccgggacggccgctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgactagtctcgagtcttgtgtgcctggcatatgataggcatttaatagttttaaagaattaatgtatttagatgaattgcataccaaatctgctgtcttttctttatggcttcattaacttaatttgagagaaattaattattctgcaacttagggacaagtcatctctttgaatattctgtagtttgaggagaatatttgttatatttgcaaaataaaataagtttgcaagttttttttttctgccccaaagagctctgtgtccttgaacataaaatacaaataaccgctatgctgttaattattggcaaatgtcccattttcaacctaaggaaataccataaagtaacagatataccaacaaaaggttactagttaacaggcattgcctgaaaagagtataaaagaatttcagcatgattttccatattgtgcttccaccactgccaataacaaaataactagcagagctagcctcgaggctagc

SEQ ID NO: 279 - DNA, 770 bp, synthetic construct
  source 1..770: mol_type = other DNA
aattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgaatgcatactagtaacatttctctggcctaactggccggtaccacctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgattaccggtcgacgctagc

SEQ ID NO: 280 - DNA, 11 bp, synthetic construct
  source 1..11: mol_type = other DNA
tacttggcaaa

SEQ ID NO: 281 - DNA, 537 bp, synthetic construct
  source 1..537: mol_type = other DNA
aattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgaatgcatactagtaacatttctctggcctaactggccggtaccatgacccacgtgatgctgagaagtactcctgccctaggaagagactcagggcagagggaggaaggacagcagaccagacagtcacagcagccttgacaaaacgttcctggaactaccggtcgacgctagc

SEQ ID NO: 282 - DNA, 613 bp, synthetic construct
  source 1..613: mol_type = other DNA
aattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgaatgcatactagtaacatttctctggcctaactggccggtaccagtggtgggggagtgaaaagagagatggagaaagaggggatgggcagaaagaggaggaggagtcaggggcagggcatggaggtgggtggggctgggctgccaaagcaggataaatgcacacctgcctgctggtctgggctccctgcctcgggctctcaccctcctctcctgcagctccagctttgtgctctccggtcgacgctagc

SEQ ID NO: 283 - DNA, 588 bp, synthetic construct
  source 1..588: mol_type = other DNA
aattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgaatgcatactagtaacatttctctggcctaactggccggtaccgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctgaaccggtcgacgctagc

SEQ ID NO: 284 - DNA, 673 bp, synthetic construct
  source 1..673: mol_type = other DNA
aattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcaatgcatactagtaacatttctctggcctaactggccggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctcttaaccggtcgacgctagc

SEQ ID NO: 285 - DNA, 2615 bp, synthetic construct
  source 1..2615: mol_type = other DNA
gagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatgaatgcatgaattcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcccgggacggccgctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgactagtaacatttctctggcctaactggccggtaccacatcggctatgctgctgctaatgccacgtcaccacatcgacatgccacgtcaccatcatgccatgccacgtcaccactgcaagatgccacgtcaccacagtataatgccacgtcaccaagttactatgccacgtcaccaggtacctgcgctcccgacatgccccgcggcgcgccattaaccgccagatttgagtcgcgggacccgttggcagaggtggaccggtcgacgctagc

SEQ ID NO: 286 - DNA, 14 bp, synthetic construct
  source 1..14: mol_type = other DNA
ttttggcgcccttt

SEQ ID NO: 287 - DNA, 11 bp, synthetic construct
  source 1..11: mol_type = other DNA
tcctttgattt

SEQ ID NO: 288 - DNA, 17 bp, synthetic construct
  source 1..17: mol_type = other DNA
tgaacaggattaatgta

SEQ ID NO: 289 - DNA, 1809 bp, synthetic construct
  source 1..1809: mol_type = other DNA
cgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccactagttattaatagtaatcaattacggggtcattagttcatagcccatatatggagttccgcgttacataacttacggtaaatggcccgccttgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccatggatctcagattgaattatttgcctgtcatacagctaataattgaccataagacaattagatttaaattagttttgaatctttctaataccaaagttcagtttactgttccatgttgcttctgagtggcttcacagacttatgaaaaagtaaacggaatcagaattacatcaatgcaaaagcattgctgtgaactctgtacttaggactaaactttgagcaataacacatatagattgaggattgtttgctgttagtatacaaactctggttcaaagctcctctttattgcttgtcttggaaaatttgctgttcttcatggtttctcttttcactgctatctatttttctcaaccactcacatggctacaataactgtctgcaagcttatgattcccaaatatctatctctagcctcaatcttgttccagaagataaaaagtagtattcaaatgcacatcaacgtctccacttggagggcttaaagacgtttcaacatacaaaccggggagttttgcctggaatgtttcctaaaatgtgtcctgtagcacatagggtcctcttgttccttaaaatctaattacttttagcccagtgctcatcccacctatggggagatgagagtgaaaagggagcctgattaataattacactaagtcaataggcatagagccaggactgtttgggtaaactggtcactttatcttaaactaaatatatccaaaactgaacatgtacttagttactaagtctttgactttatctcattcataccactcagctttatccaggccacttatttgacagtattattgcgaaaacttcctactagtgtcatctctttgaatattctgtagtttgaggagaatatttgttatattgcacaataaaataagtttgcaagttttttttttctgccccaaagagctctgtgtccttgaacataaaatacaaataaccgctatgctgttaattattaacaaatgtcccattttcaacctaaggaaataccataaagtaacagatataccaacaaaaggttaataattaacaggcattgcctgaaaagagtataaaaggctttcagcatgattttccatattgtgcttccaccactgccaataacaaaccggtcgacgctagc

SEQ ID NO: 290 - DNA, 3243 bp, synthetic construct
  source 1..3243: mol_type = other DNA
actggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatgaatgcatgaattcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcccgggacggccgctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgactagtaacatttctctggcctaactggccggtacctgccactcaaagtggcacactccctgctcaggaggccgggagggaggacacagccctggcaactcctctgccccggggggtcaggaaggggtcaccccacactccagaaccctacagaatgtggccttggcttttcccatcaagagctggggaaagccaggccccgacttcattaccccctgcccccgtcccatgctcagtgggccccatcgtgggtccatgccacactcccaactgagcagccccgcagccccgcgtgtcacagacatggggcctcctaattgctgctgaggtcccaatccctggctggacgtgcctgatggaagagccagctctggtctcagggggctggtttgcaggagtctccacagacctggctccagctttgtgtcttcaaatgaatacccggccaagattgcaactaaattaccagaaacacttaggtttcctcacagactccacaacagggatggagaaggaagtcagctgacgaggttacgacgctgttcgagggagtctttcttgggtcacaagtggtaaactgtgttccctgaacaaaaccaggaagctttcagtgtttattgtatgtactaagtggagggaggggcttcagattctgataaaaatatctccccattcccagtgcccaatgtgacatgaataggagggcccctccctgaattcccaagcagatctccagagacagcttcagagagcagggagcccacggtggctggggctttagggactttctgggttgtggggaggctagaggctgggcagtcccagcaggatttggcctctagggaccgggcactgtagggctcaggagagcagctgccgtcccagtatataagcataggtggaattatctggaaacatatttctgcgtttcacaggcagagaaatcagtctatccctaaagaatggaagagctacagtagcagacctaccaccctccaccctcccacaggcaaaagcccctgagattcaggtttgggaagaaaaagaaaatatcccaaatatgtcatttgagaaagcagctgctaaccacaggcggccccagcttttctcaagatccaggatgtgggttcagtgcccttactagggcagtgggggaggacggtcagtaccaggaccccaggcacaggcctggaggacttgctcccccaagcaactcagatccacgcagaacccatggtaccactagtggtgactcatgggtgactcatgggtgactcatgggtgactcatgggtgactcatgggtgactcatgggtgactcatgggtgactcatgggtgactcatgtgcgctcccgacatgccccgcggcgcgccattaaccgccagatttgagtcgcgggacccgttggcagaggtggaccggtcgacgctagc

SEQ ID NO: 291 - DNA, 2602 bp, synthetic construct
  source 1..2602: mol_type = other DNA
cgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatgaatgcatgaattcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcccgggacggccgctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgactagtcttctgccctgagaaagacctatgattgcatgacacaaaagagactgttcaaagggacaccatcattcagcagggcaagcctccttgctgggggcaacctggtagctcctgagcctccctcatcttcactgagcccctccaactctctgagttcccatgcccctcactgaacctcccttcccccatggcgagcctccgccagcacctttgcacacactcagccccttccccctactgagccccagcacagtcactgaacagctcttcttcccctctgactgagtcatcctcccaagccctccccttcccctcactgagtctccaccacccctggtcactgggcaccctgcttctgacctcctccctcccccaacccctccacccttcctcttcactgagcctggcgcctctcacccacccgccttcctctcccagccgcttctgagctgcctctttggagcccaactgtctcgcccacgagtccccatcactcagtctcactcactctaagacacctgaaagcagttagagaacatgtgttcatggggggaggatgaggctctatcatcatcctgcaaactagtgtccccacccacacattcctgtccccacccacacattcctgtccccacccacacattcctgtccccacccacacattcctgtccccacccacacattcctgtccccacccacacattcctgaccggtcgacgctagc

SEQ ID NO: 292 - DNA, 2543 bp, synthetic construct
  source 1..2543: mol_type = other DNA
cgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatgaatgcatgaattcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcccgggacggccgctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgactagtgaacatacacacctgtgggggtgtctaaggggctcccagggagttctggggggtcctggggagcaggaccctcttcactccctcctccaggggaagtggccctggggcaccccaggctgttcccccagctctgtggggccgaagccatccacagggggctttccccaccggatgtggtgcgggccgtggttaatctcacttgagttagtcacccaggacaaacagctaaccgacacaattcctcccaagtccagggggccggaggcggggtcagcacctggcggcaggagacagtgctgccctgggatgtggccgggcctccctccattcccaatcctgttgtctctgtggcaatacctggctgggagctcctatcaggcccgtgacccccgccctttctccagtgccctcctgtctgcattcacctgtcagatcccggggagagaggggcactggcggccgcccaggaccagagctgtggggcctcccgcaccagagtgcagtgaaggtttgtgggctgctagtgtccccacccacacattcctgtccccacccacacattcctgtccccacccacacattcctgtccccacccacacattcctgtccccacccacacattcctgtccccacccacacattcctgaccggtcgacgctagc

SEQ ID NO: 293 - DNA, 2585 bp, synthetic construct
  source 1..2585: mol_type = other DNA
gagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatgaatgcatgaattcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcccgggacggccgctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgactagtaacatttctctggcctaactggccggtaccactagtaccggaagtaagaaccggaagtatcgaccggaagtagacaccggaagtactaaccggaagtaactaccggaagtatgcaccggaagtatgcgctcccgacatgccccgcggcgcgccattaaccgccagatttgagtcgcgggacccgttggcagaggtggaccggtcgacgctagc

SEQ ID NO: 294 - DNA, 14 bp, synthetic construct
  source 1..14: mol_type = other DNA
atgccacgtaatca

SEQ ID NO: 295 - DNA, 12 bp, synthetic construct
  source 1..12: mol_type = other DNA
gagaacaaagaa

SEQ ID NO: 296 - DNA, 10 bp, synthetic construct
  source 1..10: mol_type = other DNA
atcggaagtg

SEQ ID NO: 297 - DNA, 11 bp, synthetic construct
  source 1..11: mol_type = other DNA
gggtgactcat

SEQ ID NO: 298 - DNA, 12 bp, synthetic construct
  source 1..12: mol_type = other DNA
gcgaccaccgaa

SEQ ID NO: 299 - DNA, 11 bp, synthetic construct
  source 1..11: mol_type = other DNA
gaaatctgagc

SEQ ID NO: 300 - DNA, 11 bp, synthetic construct
  source 1..11: mol_type = other DNA
tcctttgatgt

SEQ ID NO: 301 - DNA, 924 bp, synthetic construct
  source 1..924: mol_type = other DNA
tcaaacatgagagcttagtacgtgaaacatgagagcttagtacgttagccatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgaatgcatactagtaacatttctctggcctaactggccggtaccgatcttgatatcctcgaggctagcatgatcaccatgagtcacccatgagtcacccatgagtcacccatgagtcacccatgagtcacccatgagtcacccatgagtcacccatgagtcacccatgagtcaccactagtggtaccacctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgattaccggtcgacgctagc

SEQ ID NO: 302 - DNA, 644 bp, synthetic construct
  source 1..644: mol_type = other DNA
aattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgaatgcatactagtggtgactcatgggtgactcatgggtgactcatgggtgactcatgggtgactcatgggtgactcatgggtgactcatgggtgactcatgggtgactcatggtgatcatgctagcctcgaggatatcaagatcggtaccatgacccacgtgatgctgagaagtactcctgccctaggaagagactcagggcagagggaggaaggacagcagaccagacagtcacagcagccttgacaaaacgttcctggaactaccggtcgacgctagc

SEQ ID NO: 303 - DNA, 779 bp, synthetic construct
  source 1..779: mol_type = other DNA
aattttattgttcaaacatgagagcttagtacgtgaaacatgagagcttagtacgttagccatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgaatgcatactagtaacatttctctggcctaactggccggtaccgatcttgatatcctcgaggctagcatgatcaccatgagtcacccatgagtcacccatgagtcacccatgagtcacccatgagtcacccatgagtcacccatgagtcacccatgagtcacccatgagtcaccactagtggtaccagtggtgggggagtgaaaagagagatggagaaagaggggatgggcagaaagaggaggaggagtcaggggcagggcatggaggtgggtggggctgggctgccaaagcaggataaatgcacacctgcctgctggtctgggctccctgcctcgggctctcaccctcctctcctgcagctccagctttgtgctctaccggtcgacgctagc

SEQ ID NO: 304 - DNA, 724 bp, synthetic construct
  source 1..724: mol_type = other DNA
aattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgactagtaacatttctctggcctaactggccggtaccactagtggtgactcatgggtgactcatgggtgactcatgggtgactcatgggtgactcatgggtgactcatgggtgactcatgggtgactcatgggtgactcatggtgatcatgctagcctcgaggatatcaagatcggtaccgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctgaaccggtcgacgctagc

SEQ ID NO: 305 - DNA, 1697 bp, synthetic construct
  source 1..1697: mol_type = other DNA
ataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatgaatgcatgaattcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcccgggacggccgctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgaatgcatactagtggtgactcatgggtgactcatgggtgactcatgggtgactcatgggtgactcatgggtgactcatgggtgactcatgggtgactcatgggtgactcatggtgatcatgctagcctcgaggatatcaagatcggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctcttccggtcgacgctagc

SEQ ID NO: 306 - DNA, 11 bp, synthetic construct
  source 1..11: mol_type = other DNA
tacttggcata

SEQ ID NO: 307 - DNA, 11 bp, synthetic construct
  source 1..11: mol_type = other DNA
ggatgactcac

SEQ ID NO: 308 - DNA, 11 bp, synthetic construct
  source 1..11: mol_type = other DNA
ttctaatctct

SEQ ID NO: 309 - DNA, 11 bp, synthetic construct
  source 1..11: mol_type = other DNA
gggtgactcac

SEQ ID NO: 310 - DNA, 1128 bp, synthetic construct
  source 1..1128: mol_type = other DNA
cttataaaacttaaaaccttagaggctatttaagttgctgatttatattaattttattgttcaaacatgagagcttagtacgtgaaacatgagagcttagtacgttagccatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgaatgcatactagtaacatttctctggcctaactggccggtaccgatcttgatatcctcgaggctagcatgatcaccatgagtcacccatgagtcacccatgagtcacccatgagtcacccatgagtcacccatgagtcacccatgagtcacccatgagtcacccatgagtcaccactagtggtaccgattcttgatatcctcgaggctagcatgatcaccatgagtcacccatgagtcacccatgagtcacccatgagtcacccatgagtcacccatgagtcacccatgagtcacccatgagtcacccatgagtcaccactagtggtaccacctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgattaccggtcgacgctagc

SEQ ID NO: 311 - DNA, 823 bp, synthetic construct
  source 1..823: mol_type = other DNA
aattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgaatgcatactagtaacatttctctggcctaactggccggtaccactagtggtgactcatgggtgactcatgggtgactcatgggtgactcatgggtgactcatgggtgactcatgggtgactcatgggtgactcatgggtgactcatggtgatcatgctagcctcgaggatatcaagatcggtaccactagtggtgactcatgggtgactcatgggtgactcatgggtgactcatgggtgactcatgggtgactcatgggtgactcatgggtgactcatgggtgactcatggtgatcatgctagcctcgaggatatcaagatcggtaccatgacccacgtgatgctgagaagtactcctgccctaggaagagactcagggcagagggaggaaggacagcagaccagacagtcacagcagccttgacaaaacgttcctggaactaccggtcgacgctagc

SEQ ID NO: 312 - DNA, 1216 bp, synthetic construct
  source 1..1216: mol_type = other DNA
cttataaaacttaaaaccttagaggctatttaagttgctgatttatattaattttattgttcaaacatgagagcttagtacgtgaaacatgagagcttagtacgttagccatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgaatgcatactagtaacatttctctggcctaactggccggtaccgatcttgatatcctcgaggctagcatgatcaccatgagtcacccatgagtcacccatgagtcacccatgagtcaccactagtggtaccgatcttgatatcctcgaggctagcatgatcaccatgagtcacccatgagtcacccatgagtcacccatgagtcacccatgagtcacccatgagtcacccatgagtcacccatgagtcacccatgagtcaccactagtggtaccgattcttgatatcctcgaggctagcatgatcaccatgagtcacccatgagtcacccatgagtcacccatgagtcacccatgagtcacccatgagtcacccatgagtcacccatgagtcacccatgagtcaccactagtggtaccacctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgattaccggtcgacgctagc

SEQ ID NO: 313 - DNA, 11 bp, synthetic construct
  source 1..11: mol_type = other DNA
ggatgactcag

SEQ ID NO: 314 - DNA, 10 bp, synthetic construct
  source 1..10: mol_type = other DNA
gacacgtgtc

SEQ ID NO: 315 - DNA, 1395 bp, synthetic construct
  source 1..1395: mol_type = other DNA
ctgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatgaattgggatcttcacacagcaggtaaggttgcgggccgggcctgggccgggtccgggccggggcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaccgcactgacccctggtgttgctttttttttttaggccgcaagctgaagcgtgtccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggtaccatgcatactagtggtgactcatgggtgactcatgggtgactcatgggtgactcatgggtgactcatgggtgactcatgggtgactcatgggtgactcatgggtgactcatgcggtgctagctataaaaggccagcagcagcctgaccacatctcatcctcctcgaggatatcaagatctggcctcggcggccagaattcaccggtcacc

SEQ ID NO: 316 - DNA, 10 bp, synthetic construct
  source 1..10: mol_type = other DNA
ggcacgtgta

SEQ ID NO: 317 - DNA, 10 bp, synthetic construct
  source 1..10: mol_type = other DNA
ggcacgtgtc

SEQ ID NO: 318 - DNA, 814 bp, synthetic construct
  source 1..814: mol_type = other DNA
ggccgctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccactagtggtgactcatgggtgactcatgggtgactcatgggtgactcatgggtgactcatgggtgactcatgggtgactcatgggtgactcatgactagtgtccccacccacacattcctgtccccacccacacattcctgtccccacccacacattcctgtccccacccacacattcctgtccccacccacacattcctgtccccacccacacattcctgtgcgctcccgacatgccccgcggcgcgccattaaccgccagatttgagtcgcgggacccgttggcagaggtgggctagcctcgaggatatcaagatctggcctcggcggccaagcttgctagc

SEQ ID NO: 319 - DNA, 558 bp, synthetic construct
  source 1..558: mol_type = other DNA
aattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgactagtggtgactcatgggtgactcatgggtgactcatgggtgactcatgggtgactcatgggtgactcatgggtgactcatgggtgactcatgggtgactcatgtgcgctcccgacatgccccgcggcgcgccattaaccgccagatttgagtcgcgggacccgttggcagaggtggaccggtcgacgctagc

SEQ ID NO: 320 - DNA, 14 bp, synthetic construct
  source 1..14: mol_type = other DNA
atgccacgtcaaca

SEQ ID NO: 321 - DNA, 10 bp, synthetic construct
  source 1..10: mol_type = other DNA
ggcacgtgtt

SEQ ID NO: 322 - DNA, 10 bp, synthetic construct
  source 1..10: mol_type = other DNA
ggcacgtgcc

SEQ ID NO: 323 - DNA, 12 bp, synthetic construct
  source 1..12: mol_type = other DNA
aaaaccggttct

SEQ ID NO: 324 - DNA, 885 bp, synthetic construct
  source 1..885: mol_type = other DNA
gacggccgctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgactagtggtgactcatgggtgactcatgggtgactcatgggtgactcatgggtgactcatgggtgactcatgggtgactcatgggtgactcatgggtgactcatgcatggggcggggtgatgacacagcaattcgggactttccacgcttgcgtgagaagagaccggaagtgaatgacacagcaattcgcttgcgtgagaagctgggactttcctaggggcggggttgggactttccacatgacacagcaatacaacgcgtcccgacatgccccgcggcgcgccattaaccgccagatttgagtcgcgggacccgttggcagaggtgggaattcaccggtcgacgctagc

SEQ ID NO: 325 - DNA, 597 bp, synthetic construct
  source 1..597: mol_type = other DNA
tttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgactagtggtgactcatgggtgactcatgggtgactcatgggtgactcatgggtgactcatgggtgactcatgggtgactcatgggtgactcatgggtgactcatgcataccggaagtacttgcgcaatgaccggaagtacaacgcgtcccgacatgccccgcggcgcgccattaaccgccagatttgagtcgcgggacccgttggcagaggtgggaattcaccggtcgacgctagc

SEQ ID NO: 326 - DNA, 659 bp, synthetic construct
  source 1..659: mol_type = other DNA
taattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgactagtggtgactcatgggtgactcatgggtgactcatgggtgactcatgggtgactcatgggtgactcatgggtgactcatgggtgactcatgggtgactcatgcatttgcgcaacaggggcggggtgatgacacagcaattcgcttgcgtgagaagagaccggaagtgagggactttccacatgacacagcaatacaacgcgtcccgacatgccccgcggcgcgccattaaccgccagatttgagtcgcgggacccgttggcagaggtgggaattcaccggtcgacgctagc

SEQ ID NO: 327 - DNA, 21 bp, synthetic construct
  source 1..21: mol_type = other DNA
ataaataaaaatggactaatt

SEQ ID NO: 328 - DNA, 660 bp, synthetic construct
  source 1..660: mol_type = other DNA
gcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccactagtggtgactcatgggtgactcatgggtgactcatgggtgactcatgggtgactcatgggtgactcatgggtgactcatgggtgactcatgggtgactcatgcggtgctagctataaaaggccagcagcagcctgaccacatctcatcctcctcgaggatatcaagatctggcctcggcggccaagcttgctagc

SEQ ID NO: 329 - DNA, 885 bp, synthetic construct
  source 1..885: mol_type = other DNA
aattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgactagtggggcggggtgatgacacagcaattcgggactttccacgcttgcgtgagaagagaccggaagtgaatgacacagcaattcgcttgcgtgagaagctgggactttcctaggggcggggttgggactttccacatgacacagcaatacagtaccacctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgattaccggtcgacgctagc

SEQ ID NO: 330 - DNA, 242 bp, synthetic construct
  source 1..242: mol_type = other DNA
ggggcggggtgatgacacagcaattcgggactttccacgcttgcgtgagaagagaccggaagtgaatgacacagcaattcgcttgcgtgagaagctgggactttcctaggggcggggttgggactttccacatgacacagcaatacaacgcgtcccgacatgccccgcggcgcgccattaaccgccagatttgagtcgcgggacccgttggcagaggtgggaattcaccggtcgacgctagc

SEQ ID NO: 331 - DNA, 682 bp, synthetic construct
  source 1..682: mol_type = other DNA
aattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgactagtggggcggggtgatgacacagcaattcgggactttccacgcttgcgtgagaagagaccggaagtgaatgacacagcaattcgcttgcgtgagaagctgggactttcctaggggcggggttgggactttccacatgacacagcaatacactagtaacatttctctggcctaactggccggtaccatgacccacgtgatgctgagaagtactcctgccctaggaagagactcagggcagagggaggaaggacagcagaccagacagtcacagcagccttgacaaaacgttcctggaactaccggtcgacgctagc

SEQ ID NO: 332 - DNA, 759 bp, synthetic construct
  source 1..759: mol_type = other DNA
aattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgactagtggggcggggtgatgacacagcaattcgggactttccacgcttgcgtgagaagagaccggaagtgaatgacacagcaattcgcttgcgtgagaagctgggactttcctaggggcggggttgggactttccacatgacacagcaatacactagtaacatttctctggcctaactggccggtaccagtggtgggggagtgaaaagagagatggagaaagaggggatgggcagaaagaggaggaggagtcaggggcagggcatggaggtgggtggggctgggctgccaaagcaggataaatgcacacctgcctgctggtctgggctccctgcctcgggctctcaccctcctctcctgcagctccagctttgtgctctaccggtcgacgctagc

SEQ ID NO: 333 - DNA, 733 bp, synthetic construct
  source 1..733: mol_type = other DNA
aattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgactagtggggcggggtgatgacacagcaattcgggactttccacgcttgcgtgagaagagaccggaagtgaatgacacagcaattcgcttgcgtgagaagctgggactttcctaggggcggggttgggactttccacatgacacagcaatacactagtaacatttctctggcctaactggccggtaccgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctgaaccggtcgacgctagc

SEQ ID NO: 334 - DNA, 630 bp, synthetic construct
  source 1..630: mol_type = other DNA
aggccgcaagctgaagcgtgtccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggtaccatgcatactagtggggcggggtgatgacacagcaattcgggactttccacgcttgcgtgagaagagaccggaagtgaatgacacagcaattcgcttgcgtgagaagctgggactttcctaggggcggggttgggactttccacatgacacagcaatacactagtaacatttctctggcctaactggccggtaccgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctgaagaattcaccggtcacc

SEQ ID NO: 335 - DNA, 819 bp, synthetic construct
  source 1..819: mol_type = other DNA
aattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgactagtggggcggggtgatgacacagcaattcgggactttccacgcttgcgtgagaagagaccggaagtgaatgacacagcaattcgcttgcgtgagaagctgggactttcctaggggcggggttgggactttccacatgacacagcaatacactagtaacatttctctggcctaactggccggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctcttaaccggtcgacgctagc

SEQ ID NO: 336 - DNA, 10 bp, synthetic construct
  source 1..10: mol_type = other DNA
accggatgtg

SEQ ID NO: 337 - DNA, 11 bp, synthetic construct
  source 1..11: mol_type = other DNA
gggcgggaccg

SEQ ID NO: 338 - DNA, 11 bp, synthetic construct
  source 1..11: mol_type = other DNA
gggtgactcag

SEQ ID NO: 339 - DNA, 16 bp, synthetic construct
  source 1..16: mol_type = other DNA
aactgaaacttgatac

SEQ ID NO: 340 - DNA, 13 bp, synthetic construct
  source 1..13: mol_type = other DNA
attgcagatgttt

SEQ ID NO: 341 - DNA, 16 bp, synthetic construct
  source 1..16: mol_type = other DNA
aactgtaacctgatac

SEQ ID NO: 342 - DNA, 2637 bp, synthetic construct
  source 1..2637: mol_type = other DNA
gagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatgaatgcatgaattcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcccgggacggccgctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgactagtaacatttctctggcctaactggccggtacccgatgtagctgagcgacagtatagtgcacagtgactgcagcagtcattatacgtcgcctaaatcgagatgctgtactgatctataaggatcggtaatgacgtaatgacgtaatgacgtaatgacgtaatgacgtaatgacggtacctgcgctcccgacatgccccgcggcgcgccattaaccgccagatttgagtcgcgggacccgttggcagaggtggaccggtcgacgctagc

SEQ ID NO: 343 - DNA, 2631 bp, synthetic construct
  source 1..2631: mol_type = other DNA
aactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatgaatgcatgaattcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcccgggacggccgctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacggccgcggtgccagggcgtgcccttgggctccccgggcgcgactagtaacatttctctggcctaactggccggtaccagctgagcgacagtatagtgcacagtgactgcagcagtcattatacgtcgcctaaatcgagatgctgtactgatctataagtcgtaaactgtcgtaaactgtcgtaaactgtcgtaaactgtcgtaaactgtcgtaaactggtacctgcgctcccgacatgccccgcggcgcgccattaaccgccagatttgagtcgcgggacccgttggcagaggtggaccggtcgacgctagc

SEQ ID NO: 344 - DNA, 137 bp, synthetic construct
  source 1..137: mol_type = other DNA
aataggtaccactagtggttttgtggggttttgtggggttttgtggggttttgtggggttttgtggggttttgtggggttttgtggggttttgtggggttttgtggggttttgtggtgcgctcccgacatgccccgc

SEQ ID NO: 345 - DNA, 137 bp, synthetic construct
  source 1..137: mol_type = other DNA
aataggtaccactagtagttcaacacgtggtctgggagttcaacacgtggtctgggagttcaacacgtggtctgggagttcaacacgtggtctgggagttcaacacgtggtctgggtgcgctcccgacatgccccgc

SEQ ID NO: 346 - DNA, 138 bp, synthetic construct
  source 1..138: mol_type = other DNA
aataggtaccactagtggttttgtggagaggttttgtggtcgggttttgtgggacggttttgtggctaggttttgtggactggttttgtggtgcggttttgtgggtaggttttgtggtgcgctcccgacatgccccgc

SEQ ID NO: 347 - DNA, 149 bp, synthetic construct
  source 1..149: mol_type = other DNA
aataggtaccactagtagttcaacacgtggtctgggagaagttcaacacgtggtctgggtcgagttcaacacgtggtctggggacagttcaacacgtggtctgggctaagttcaacacgtggtctgggtgcgctcccgacatgccccgc

SEQ ID NO: 348 - DNA, 135 bp, synthetic construct
  source 1..135: mol_type = other DNA
aataggtaccactagtagccacttgaaattagccacttgaaattagccacttgaaattagccacttgaaattagccacttgaaattagccacttgaaattagccacttgaaatttgcgctcccgacatgccccgc

SEQ ID NO: 349 - DNA, 141 bp, synthetic construct
  source 1..141: mol_type = other DNA
aataggtaccactagtgacagataagaaagacagataagaaagacagataagaaagacagataagaaagacagataagaaagacagataagaaagacagataagaaagacagataagaaatgcgctcccgacatgccccgc

SEQ ID NO: 350 - DNA, 136 bp, synthetic construct
  source 1..136: mol_type = other DNA
aataggtaccactagtagccacttgaaattagaagccacttgaaatttcgagccacttgaaattgacagccacttgaaattctaagccacttgaaattactagccacttgaaatttgcgctcccgacatgccccgc

SEQ ID NO: 351 - DNA, 146 bp, synthetic construct
  source 1..146: mol_type = other DNA
aataggtaccactagtgacagataagaaaagagacagataagaaatcggacagataagaaagacgacagataagaaactagacagataagaaaactgacagataagaaatgcgacagataagaaatgcgctcccgacatgccccgc

SEQ ID NO: 352 - DNA, 141 bp, synthetic construct
  source 1..141: mol_type = other DNA
aataggtaccactagtctgggaacaagtgctgggaacaagtgctgggaacaagtgctgggaacaagtgctgggaacaagtgctgggaacaagtgctgggaacaagtgctgggaacaagtgtgcgctcccgacatgccccgc

SEQ ID NO: 353 - DNA, 136 bp, synthetic construct
  source 1..136: mol_type = other DNA
aataggtaccactagtttctaatctatttctaatctatttctaatctatttctaatctatttctaatctatttctaatctatttctaatctatttctaatctatttctaatctattgcgctcccgacatgccccgc

SEQ ID NO: 354 - DNA, 146 bp, synthetic construct
  source 1..146: mol_type = other DNA
aataggtaccactagtctgggaacaagtgagactgggaacaagtgtcgctgggaacaagtggacctgggaacaagtgctactgggaacaagtgactctgggaacaagtgtgcctgggaacaagtgtgcgctcccgacatgccccgc

SEQ ID NO: 355 - DNA, 132 bp, synthetic construct
  source 1..132: mol_type = other DNA
aataggtaccactagtttctaatctatagattctaatctattcgttctaatctatgacttctaatctatctattctaatctatactttctaatctattgcttctaatctattgcgctcccgacatgccccgc

SEQ ID NO: 356 - DNA, 141 bp, synthetic construct
  source 1..141: mol_type = other DNA
aataggtaccactagtgactcctcaaggggactcctcaaggggactcctcaaggggactcctcaaggggactcctcaaggggactcctcaaggggactcctcaaggggactcctcaagggtgcgctcccgacatgccccgc

SEQ ID NO: 357 - DNA, 136 bp, synthetic construct
  source 1..136: mol_type = other DNA
aataggtaccactagtggtgactcatgggtgactcatgggtgactcatgggtgactcatgggtgactcatgggtgactcatgggtgactcatgggtgactcatgggtgactcatgtgcgctcccgacatgccccgc

SEQ ID NO: 358 - DNA, 146 bp, synthetic construct
  source 1..146: mol_type = other DNA
aataggtaccactagtgactcctcaagggagagactcctcaagggtcggactcctcaaggggacgactcctcaagggctagactcctcaagggactgactcctcaagggtgcgactcctcaagggtgcgctcccgacatgccccgc

SEQ ID NO: 359 - DNA, 132 bp, synthetic construct
  source 1..132: mol_type = other DNA
aataggtaccactagtggtgactcatgagaggtgactcatgtcgggtgactcatggacggtgactcatgctaggtgactcatgactggtgactcatgtgcggtgactcatgtgcgctcccgacatgccccgc

SEQ ID NO: 360 - DNA, 142 bp, synthetic construct
  source 1..142: mol_type = other DNA
aataggtaccactagtcgggctttgatctttcgggctttgatctttcgggctttgatctttcgggctttgatctttcgggctttgatctttcgggctttgatctttcgggctttgatcttttgcgctcccgacatgccccgc

SEQ ID NO: 361 - DNA, 136 bp, synthetic construct
  source 1..136: mol_type = other DNA
aataggtaccactagtcttctgggaaacttctgggaaacttctgggaaacttctgggaaacttctgggaaacttctgggaaacttctgggaaacttctgggaaacttctgggaaatgcgctcccgacatgccccgc

SEQ ID NO: 362 - DNA, 142 bp, synthetic construct
  source 1..142: mol_type = other DNA
aataggtaccactagtcgggctttgatctttagacgggctttgatcttttcgcgggctttgatctttgaccgggctttgatctttctacgggctttgatctttactcgggctttgatcttttgcgctcccgacatgccccgc

SEQ ID NO: 363 - DNA, 132 bp, synthetic construct
  source 1..132: mol_type = other DNA
aataggtaccactagtcttctgggaaaagacttctgggaaatcgcttctgggaaagaccttctgggaaactacttctgggaaaactcttctgggaaatgccttctgggaaatgcgctcccgacatgccccgc

SEQ ID NO: 364 - DNA, 139 bp, synthetic construct
  source 1..139: mol_type = other DNA
aataggtaccactagtgcgctttgatgtgcggggcggccctttgaagttggcgctttgatgtgcggggcggccctttgaagttggcgctttgatgtgcggggcggccctttgaagttgtgcgctcccgacatgccccgc

SEQ ID NO: 365 - DNA, 135 bp, synthetic construct
  source 1..135: mol_type = other DNA
aataggtaccactagtaattcttagaaataaattcttagaaataaattcttagaaataaattcttagaaataaattcttagaaataaattcttagaaataaattcttagaaatatgcgctcccgacatgccccgc

SEQ ID NO: 366 - DNA, 145 bp, synthetic construct
  source 1..145: mol_type = other DNA
aataggtaccactagtgcgctttgatgtgcggggcggccctttgaagttgagagcgctttgatgtgcggggcggccctttgaagttgtcggcgctttgatgtgcggggcggccctttgaagttgtgcgctcccgacatgccccgc

SEQ ID NO: 367 - DNA, 136 bp, synthetic construct
  source 1..136: mol_type = other DNA
aataggtaccactagtaattcttagaaataagaaattcttagaaatatcgaattcttagaaatagacaattcttagaaatactaaattcttagaaataactaattcttagaaatatgcgctcccgacatgccccgc

SEQ ID NO: 368 - DNA, 127 bp, synthetic construct
  source 1..127: mol_type = other DNA
aataggtaccactagtaacagctgttaacagctgttaacagctgttaacagctgttaacagctgttaacagctgttaacagctgttaacagctgttaacagctgtttgcgctcccgacatgccccgc

SEQ ID NO: 369 - DNA, 147 bp, synthetic construct
  source 1..147: mol_type = other DNA
aataggtaccactagtaaaacaaaggatcctttgttttaaaacaaaggatcctttgttttaaaacaaaggatcctttgttttaaaacaaaggatcctttgttttaaaacaaaggatcctttgtttttgcgctcccgacatgccccgc

SEQ ID NO: 370 - DNA, 138 bp, synthetic construct
  source 1..138: mol_type = other DNA
aataggtaccactagtaacagctgttagaaacagctgtttcgaacagctgttgacaacagctgttctaaacagctgttactaacagctgtttgcaacagctgttgtaaacagctgtttgcgctcccgacatgccccgc

SEQ ID NO: 371 - DNA, 134 bp, synthetic construct
  source 1..134: mol_type = other DNA
aataggtaccactagtaaaacaaaggatcctttgttttagaaaaacaaaggatcctttgtttttcgaaaacaaaggatcctttgttttgacaaaacaaaggatcctttgtttttgcgctcccgacatgccccgc

SEQ ID NO: 372 - DNA, 109 bp, synthetic construct
  source 1..109: mol_type = other DNA
aataggtaccactagtcacctgcacctgcacctgcacctgcacctgcacctgcacctgcacctgcacctgcacctgcacctgcacctgtgcgctcccgacatgccccgc

SEQ ID NO: 373 - DNA, 135 bp, synthetic construct
  source 1..135: mol_type = other DNA
aataggtaccactagtaaagtccaagtccaaaagtccaagtccaaaagtccaagtccaaaagtccaagtccaaaagtccaagtccaaaagtccaagtccaaaagtccaagtccatgcgctcccgacatgccccgc

SEQ ID NO: 374 - DNA, 124 bp, synthetic construct
  source 1..124: mol_type = other DNA
aataggtaccactagtcacctgagacacctgtcgcacctggaccacctgctacacctgactcacctgtgccacctgagacacctgtcgcacctggaccacctgtgcgctcccgacatgccccgc

SEQ ID NO: 375 - DNA, 136 bp, synthetic construct
  source 1..136: mol_type = other DNA
aataggtaccactagtaaagtccaagtccaagaaaagtccaagtccatcgaaagtccaagtccagacaaagtccaagtccactaaaagtccaagtccaactaaagtccaagtccatgcgctcccgacatgccccgc

SEQ ID NO: 376 - DNA, 157 bp, synthetic construct
  source 1..157: mol_type = other DNA
ccatggtggctttaccaacagtaccggattgccaagcttggccgccgaggccagatcttgatatcctcgaggctagcccacctctgccaacgggtcccgcgactcaaatctggcggttaatggcgcgccgcggggcatgtcgggagcgcaggtaccg

SEQ ID NO: 377 - DNA, 95 bp, synthetic construct
  source 1..95: mol_type = other DNA
cggagtactgtcctccgagcggagtactgtcctccgagcggagtactgtcctccgagcggagtactgtcctccgagcggagtactgtcctccgag

SEQ ID NO: 378 - DNA, 99 bp, synthetic construct
  source 1..99: mol_type = other DNA
ggtgactcatgggtgactcatgggtgactcatgggtgactcatgggtgactcatgggtgactcatgggtgactcatgggtgactcatgggtgactcatg

SEQ ID NO: 379 - DNA, 146 bp, synthetic construct
  source 1..146: mol_type = other DNA
ggggcggggtgatgacacagcaattcgggactttccacgcttgcgtgagaagagaccggaagtgaatgacacagcaattcgcttgcgtgagaagctgggactttcctaggggcggggttgggactttccacatgacacagcaatac

SEQ ID NO: 380 - DNA, 155 bp, synthetic construct
  source 1..155: mol_type = other DNA
aataggtaccactagtgtccccacccacacattcctgtccccacccacacattcctgtccccacccacacattcctgtccccacccacacattcctgtccccacccacacattcctgtccccacccacacattcctgaccggtgctagcctcgag

SEQ ID NO: 381 - DNA, 155 bp, synthetic construct
  source 1..155: mol_type = other DNA
ctgagcgacagtatagtgcacagtgactgcagcagtcattcctttgatgtacgcaactcctttgatgtctatgcgtcctttgatgttaaggattcctttgatgtaggtacatcctttgatgtccgtaaatcctttgatgtgacgatcttgatatc

SEQ ID NO: 382 - DNA, 140 bp, synthetic construct
  source 1..140: mol_type = other DNA
tacctgatcaaacatgcccggacatgtcgtaagacataaacatgcccggacatgtcctcgcaatctaacatgcccggacatgtcctcgcaatctaacatgcccggacatgtctgcaagctacaacatgcccggacatgtc

SEQ ID NO: 383 - DNA, 78 bp, synthetic construct
  source 1..78: mol_type = other DNA
ggggcggggtgatgacacagcaattcgggactttccacgcttgcgtgagaagagaccggaagtgaatgacacagcaat

SEQ ID NO: 384 - DNA, 66 bp, synthetic construct
  source 1..66: mol_type = other DNA
gcttgcgtgagaagctgggactttcctaggggcggggttgggactttccacatgacacagcaatac

SEQ ID NO: 385 - DNA, 122 bp, synthetic construct
  source 1..122: mol_type = other DNA
ggtgactcatgggtgactcatgggtgactcatgctacgtgtgacggtgactcatgggtgactcatgggtgactcatgaagtcgcagattggtgactcatgggtgactcatgggtgactcatg

SEQ ID NO: 386 - DNA, 123 bp, synthetic construct
  source 1..123: mol_type = other DNA
ggtgactcatgatgatgccacgtcaccaatgccacgtcaccaggtgactcatgggtgactcatgacgtgtgacatgccacgtcaccaatgccacgtcaccaggtgactcatgggtgactcatg

SEQ ID NO: 387 - DNA, 117 bp, synthetic construct
  source 1..117: mol_type = other DNA
gggaggaagtcgtaaaacttgggaggaagtcgtaaaaaatgggaggaagtcgtaaaatgcgggaggaagtcgtaaaagaagggaggaagtcgtaaaaatcgggaggaagtcgtaaaa

SEQ ID NO: 388 - DNA, 140 bp, synthetic construct
  source 1..140: mol_type = other DNA
atgactcagcaattagcgagttagaatgactcagcaattatgcgtcggacatgactcagcaattacatctcgattatgactcagcaattaggataggcatatgactcagcaattacatagcagcaatgactcagcaatta

SEQ ID NO: 389 - DNA, 101 bp, synthetic construct
  source 1..101: mol_type = other DNA
acatcaaaggatttacggacatcaaaggatgtacctacatcaaaggaatccttaacatcaaaggacgcatagacatcaaaggagttgcgtacatcaaagga

SEQ ID NO: 390 - DNA, 99 bp, synthetic construct
  source 1..99: mol_type = other DNA
cacttccggtttacttccacttccggtttactagcacttccggtttacgctcacttccggtttacgatcacttccggtttacagacacttccggtttac

SEQ ID NO: 391 - DNA, 124 bp, synthetic construct
  source 1..124: mol_type = other DNA
gcgtccgcccgagtccccgcctcgccgccaacgccaatgctcatgcgtccgcccgagtccccgcctcgccgccaacgccatcatgcctgcgtccgcccgagtccccgcctcgccgccaacgcca

SEQ ID NO: 392 - DNA, 87 bp, synthetic construct
  source 1..87: mol_type = other DNA
caacatggcggcgcccaacatggcggctaccaacatggcggcctccaacatggcggcaggcaacatggcggctgccaacatggcggc

SEQ ID NO: 393 - DNA, 71 bp, synthetic construct
  source 1..71: mol_type = other DNA
tggttgctgactaattgagatgcatgctttgcatacttctgcctgctggggagcctggggactttccacac

SEQ ID NO: 394 - DNA, 128 bp, synthetic construct
  source 1..128: mol_type = other DNA
gctcactcactcactcactgaggcctgcagagcaaagctctgcagtctggggacctttggtccccaggcctcagtgagtgagtgagtgagcagagagggagtggccaactccatcactaggggttcct

SEQ ID NO: 395 - DNA, 105 bp, synthetic construct
  source 1..105: mol_type = other DNA
ggtgactcatgggtgactcatgggtgactcatgggtgactcatgctacgtggtgactcatgggtgactcatgggtgactcatgggtgactcatgggtgactcatg

SEQ ID NO: 396 - DNA, 140 bp, synthetic construct
  source 1..140: mol_type = other DNA
agtatagtgcacagtgactgcagcagggtgactcatgatgatgccacgtcaccaatgccacgtcaccaggtgactcatgggtgactcatgatgccacgtcaccaatgccacgtcaccaggtgactcatgggtgactcatg

SEQ ID NO: 397 - DNA, 251 bp, synthetic construct
  source 1..251: mol_type = other DNA
taattgctgagtcattgctgctatgtaattgctgagtcatatgcctatcctcctttgatgtacgcaactcctttgatgtctatgcgtaattgctgagtcataatcgagatgtaattgctgagtcatgtccgacgcatcctttgatgttaaggattcctttgatgtaggtacataattgctgagtcattctaactcgctaattgctgagtcatcatctcgacctcctttgatgtccgtaaatcctttgatgt

SEQ ID NO: 398 - DNA, 178 bp, synthetic construct
  source 1..178: mol_type = other DNA
actagtggtgactcatgggtgactcatgggtgactcatgggtgactcatgggtgactcatgggtgactcatgggtgactcatgggtgactcatgggtgactcatgtgcgctcccgacatgccccgcggcgcgccattaaccgccagatttgagtcgcgggacccgttggcagaggtgg

SEQ ID NO: 399 - DNA, 191 bp, synthetic construct
  source 1..191: mol_type = other DNA
agcttgcatgcctgcaggtcggagtactgtcctccgagcggagtactgtcctccgagcggagtactgtcctccgagcggagtactgtcctccgagcggagtactgtcctccgagcggtgcgctcccgacatgccccgcggcgcgccattaaccgccagatttgagtcgcgggacccgttggcagaggtggg

SEQ ID NO: 400 - DNA, 479 bp, synthetic construct
  source 1..479: mol_type = other DNA
ctcgaggctagcatgatcaccatgagtcacccatgagtcacccatgagtcacccatgagtcacccatgagtcacccatgagtcacccatgagtcacccatgagtcacccatgagtcaccactagtggtaccacctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgatta

SEQ ID NO: 401 - DNA, 302 bp, synthetic construct
  source 1..302: mol_type = other DNA
ggtgactcatgggtgactcatgggtgactcatgggtgactcatgggtgactcatgggtgactcatgggtgactcatgggtgactcatgggtgactcatggtgatcatgctagcctcgaggatatcaagatcggtaccgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctga

SEQ ID NO: 402 - DNA, 251 bp, synthetic construct
  source 1..251: mol_type = other DNA
ggtgactcatgggtgactcatgggtgactcatgggtgactcatgggtgactcatgggtgactcatgggtgactcatgggtgactcatgggtgactcatggtgatcatgctagcctcgaggatatcaagatcggtaccatgacccacgtgatgctgagaagtactcctgccctaggaagagactcagggcagagggaggaaggacagcagaccagacagtcacagcagccttgacaaaacgttcctggaact

SEQ ID NO: 403 - DNA, 387 bp, synthetic construct
  source 1..387: mol_type = other DNA
ggtgactcatgggtgactcatgggtgactcatgggtgactcatgggtgactcatgggtgactcatgggtgactcatgggtgactcatgggtgactcatggtgatcatgctagcctcgaggatatcaagatcggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 404 - DNA, 187 bp, synthetic construct
  source 1..187: mol_type = other DNA
ggtgactcatgggtgactcatgggtgactcatgggtgactcatgggtgactcatgggtgactcatgggtgactcatgggtgactcatgggtgactcatgcggtgctagctataaaaggccagcagcagcctgaccacatctcatcctcctcgaggatatcaagatctggcctcggcggccaaattca

SEQ ID NO: 405 - DNA, 220 bp, synthetic construct
  source 1..220: mol_type = other DNA
ggggcggggtgatgacacagcaattcgggactttccacgcttgcgtgagaagagaccggaagtgaatgacacagcaattcgcttgcgtgagaagctgggactttcctaggggcggggttgggactttccacatgacacagcaatacaacgcgtcccgacatgccccgcggcgcgccattaaccgccagatttgagtcgcgggacccgttggcagaggtgg

SEQ ID NO: 406 - DNA, 499 bp, synthetic construct
  source 1..499: mol_type = other DNA
ggggcggggtgatgacacagcaattcgggactttccacgcttgcgtgagaagagaccggaagtgaatgacacagcaattcgcttgcgtgagaagctgggactttcctaggggcggggttgggactttccacatgacacagcaatacagtaccacctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgatt

SEQ ID NO: 407 - DNA, 347 bp, synthetic construct
  source 1..347: mol_type = other DNA
ggggcggggtgatgacacagcaattcgggactttccacgcttgcgtgagaagagaccggaagtgaatgacacagcaattcgcttgcgtgagaagctgggactttcctaggggcggggttgggactttccacatgacacagcaatacactagtaacatttctctggcctaactggccggtaccgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctga

SEQ ID NO: 408 - DNA, 296 bp, synthetic construct
  source 1..296: mol_type = other DNA
ggggcggggtgatgacacagcaattcgggactttccacgcttgcgtgagaagagaccggaagtgaatgacacagcaattcgcttgcgtgagaagctgggactttcctaggggcggggttgggactttccacatgacacagcaatacactagtaacatttctctggcctaactggccggtaccatgacccacgtgatgctgagaagtactcctgccctaggaagagactcagggcagagggaggaaggacagcagaccagacagtcacagcagccttgacaaaacgttcctggaact

SEQ ID NO: 409 - DNA, 433 bp, synthetic construct
  source 1..433: mol_type = other DNA
ggggcggggtgatgacacagcaattcgggactttccacgcttgcgtgagaagagaccggaagtgaatgacacagcaattcgcttgcgtgagaagctgggactttcctaggggcggggttgggactttccacatgacacagcaatacactagtaacatttctctggcctaactggccggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctta

SEQ ID NO: 410 - DNA, 193 bp, synthetic construct
  source 1..193: mol_type = other DNA
tccccacccacacattcctgtccccacccacacattcctgtccccacccacacattcctgtccccacccacacattcctgtccccacccacacattcctgtccccacccacacattcctgtgcgctcccgacatgccccgcggcgcgccattaaccgccagatttgagtcgcgggacccgttggcagaggtgg

SEQ ID NO: 411 - DNA, 288 bp, synthetic construct
  source 1..288: mol_type = other DNA
ggtgactcatgggtgactcatgggtgactcatgggtgactcatgggtgactcatgggtgactcatgggtgactcatgggtgactcatgactagtgtccccacccacacattcctgtccccacccacacattcctgtccccacccacacattcctgtccccacccacacattcctgtccccacccacacattcctgtccccacccacacattcctgtgcgctcccgacatgccccgcggcgcgccattaaccgccagatttgagtcgcgggacccgttggcagaggtgg

SEQ ID NO: 412 - DNA, 461 bp, synthetic construct
  source 1..461: mol_type = other DNA
ctgagcgacagtatagtgcacagtgactgcagcagtcattcctttgatgtacgcaactcctttgatgtctatgcgtcctttgatgttaaggattcctttgatgtaggtacatcctttgatgtccgtaaatcctttgatgtgacgtctacgtacatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgatta

SEQ ID NO: 413 - DNA, 634 bp, synthetic construct
  source 1..634: mol_type = other DNA
ctgagcgacagtatagtgcacagtgactgcagcagtcattcctttgatgtacgcaactcctttgatgtctatgcgtcctttgatgttaaggattcctttgatgtaggtacatcctttgatgtccgtaaatcctttgatgtgacgatcttgatatcctcgaggctagcatgatcaccatgagtcacccatgagtcacccatgagtcacccatgagtcacccatgagtcacccatgagtcacccatgagtcacccatgagtcacccatgagtcaccactagtggtaccacctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgatta

SEQ ID NO: 414 - DNA, 764 bp, synthetic construct
  source 1..764: mol_type = other DNA
ctgagcgacagtatagtgcacagtgactgcagcagtcattcctttgatgtacgcaactcctttgatgtctatgcgtcctttgatgttaaggattcctttgatgtaggtacatcctttgatgtccgtaaatcctttgatgtgacgtctacgtatctacctgatcaaacatgcccggacatgtcgtaagacataaacatgcccggacatgtcctcgcaatctaacatgcccggacatgtcctcgcaatctaacatgcccggacatgtctgcaagctacaacatgcccggacatgtctacaatatacgtatctacctgatcaaacatgcccggacatgtcgtaagacataaacatgcccggacatgtcctcgcaatctaacatgcccggacatgtcctcgcaatctaacatgcccggacatgtctgcaagctacaacatgcccggacatgtctacgtacatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgatta

SEQ ID NO: 415 - DNA, 412 bp, synthetic construct
  source 1..412: mol_type = other DNA
ggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 416 - DNA, 326 bp, synthetic construct
  source 1..326: mol_type = other DNA
ggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtaccgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctg

SEQ ID NO: 417 - DNA, 534 bp, synthetic construct
  source 1..534: mol_type = other DNA
ggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtgactcatgggtgactcatgggtgactcatgctacgtgtgacggtgactcatgggtgactcatgggtgactcatgaagtcgcagattggtgactcatgggtgactcatgggtgactcatgggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 418 - DNA, 535 bp, synthetic construct
  source 1..535: mol_type = other DNA
ggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtgactcatgatgatgccacgtcaccaatgccacgtcaccaggtgactcatgggtgactcatgacgtgtgacatgccacgtcaccaatgccacgtcaccaggtgactcatgggtgactcatgggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 419 - DNA, 529 bp, synthetic construct
  source 1..529: mol_type = other DNA
ggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgaggggaggaagtcgtaaaacttgggaggaagtcgtaaaaaatgggaggaagtcgtaaaatgcgggaggaagtcgtaaaagaagggaggaagtcgtaaaaatcgggaggaagtcgtaaaaggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 420 - DNA, 448 bp, synthetic construct
  source 1..448: mol_type = other DNA
ggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtgactcatgggtgactcatgggtgactcatgctacgtgtgacggtgactcatgggtgactcatgggtgactcatgaagtcgcagattggtgactcatgggtgactcatgggtgactcatgggtaccgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctg

SEQ ID NO: 421 - DNA, 449 bp, synthetic construct
  source 1..449: mol_type = other DNA
ggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtgactcatgatgatgccacgtcaccaatgccacgtcaccaggtgactcatgggtgactcatgacgtgtgacatgccacgtcaccaatgccacgtcaccaggtgactcatgggtgactcatgggtaccgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctg

SEQ ID NO: 422 - DNA, 564 bp, synthetic construct
  source 1..564: mol_type = other DNA
atgactcagcaattagcgagttagaatgactcagcaattatgcgtcggacatgactcagcaattacatctcgattatgactcagcaattaggataggcatatgactcagcaattacatagcagcaatgactcagcaattagctagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 423 - DNA, 471 bp, synthetic construct
  source 1..471: mol_type = other DNA
ggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtacccatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtatgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgatta

SEQ ID NO: 424 - DNA, 525 bp, synthetic construct
  source 1..525: mol_type = other DNA
acatcaaaggatttacggacatcaaaggatgtacctacatcaaaggaatccttaacatcaaaggacgcatagacatcaaaggagttgcgtacatcaaaggagctagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 425 - DNA, 546 bp, synthetic construct
  source 1..546: mol_type = other DNA
ggtgactcatgggtgactcatgggtgactcatgctacgtgtgacggtgactcatgggtgactcatgggtgactcatgaagtcgcagattggtgactcatgggtgactcatgggtgactcatgactagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 426 - DNA, 547 bp, synthetic construct
  source 1..547: mol_type = other DNA
ggtgactcatgatgatgccacgtcaccaatgccacgtcaccaggtgactcatgggtgactcatgacgtgtgacatgccacgtcaccaatgccacgtcaccaggtgactcatgggtgactcatgactagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 427 - DNA, 523 bp, synthetic construct
  source 1..523: mol_type = other DNA
cacttccggtttacttccacttccggtttactagcacttccggtttacgctcacttccggtttacgatcacttccggtttacagacacttccggtttacgctagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 428 - DNA, 478 bp, synthetic construct
  source 1..478: mol_type = other DNA
atgactcagcaattagcgagttagaatgactcagcaattatgcgtcggacatgactcagcaattacatctcgattatgactcagcaattaggataggcatatgactcagcaattacatagcagcaatgactcagcaattagctagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtaccgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctg

SEQ ID NO: 429 - DNA, 439 bp, synthetic construct
  source 1..439: mol_type = other DNA
acatcaaaggatttacggacatcaaaggatgtacctacatcaaaggaatccttaacatcaaaggacgcatagacatcaaaggagttgcgtacatcaaaggagctagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtaccgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctg

SEQ ID NO: 430 - DNA, 460 bp, synthetic construct
  source 1..460: mol_type = other DNA
ggtgactcatgggtgactcatgggtgactcatgctacgtgtgacggtgactcatgggtgactcatgggtgactcatgaagtcgcagattggtgactcatgggtgactcatgggtgactcatgactagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtaccgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctg

SEQ ID NO: 431 - DNA, 461 bp, synthetic construct
  source 1..461: mol_type = other DNA
ggtgactcatgatgatgccacgtcaccaatgccacgtcaccaggtgactcatgggtgactcatgacgtgtgacatgccacgtcaccaatgccacgtcaccaggtgactcatgggtgactcatgactagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtaccgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctg

SEQ ID NO: 432 - DNA, 437 bp, synthetic construct
  source 1..437: mol_type = other DNA
gtaaaccggaagtgtctgtaaaccggaagtgatcgtaaaccggaagtgagcgtaaaccggaagtgctagtaaaccggaagtggaagtaaaccggaagtgactagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtaccgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctg

SEQ ID NO: 433 - DNA, 665 bp, synthetic construct
  source 1..665: mol_type = other DNA
ggggcggggtgatgacacagcaattcgggactttccacgcttgcgtgagaagagaccggaagtgaatgacacagcaatggatccgcgtccgcccgagtccccgcctcgccgccaacgccaatgctcatgcgtccgcccgagtccccgcctcgccgccaacgccatcatgcctgcgtccgcccgagtccccgcctcgccgccaacgccaggatccgcttgcgtgagaagctgggactttcctaggggcggggttgggactttccacatgacacagcaatacctcgagggtgactcatgatgatgccacgtcaccaatgccacgtcaccaggtgactcatgggtgactcatgacgtgtgacatgccacgtcaccaatgccacgtcaccaggtgactcatgggtgactcatgggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 434 - DNA, 687 bp, synthetic construct
  source 1..687: mol_type = other DNA
atgactcagcaattagcgagttagaatgactcagcaattatgcgtcggacatgactcagcaattacatctcgattatgactcagcaattaggataggcatatgactcagcaattacatagcagcaatgactcagcaattagctagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtgactcatgatgatgccacgtcaccaatgccacgtcaccaggtgactcatgggtgactcatgacgtgtgacatgccacgtcaccaatgccacgtcaccaggtgactcatgggtgactcatgggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 435 - DNA, 648 bp, synthetic construct
  source 1..648: mol_type = other DNA
acatcaaaggatttacggacatcaaaggatgtacctacatcaaaggaatccttaacatcaaaggacgcatagacatcaaaggagttgcgtacatcaaaggagctagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtgactcatgatgatgccacgtcaccaatgccacgtcaccaggtgactcatgggtgactcatgacgtgtgacatgccacgtcaccaatgccacgtcaccaggtgactcatgggtgactcatgggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 436 - DNA, 646 bp, synthetic construct
  source 1..646: mol_type = other DNA
cacttccggtttacttccacttccggtttactagcacttccggtttacgctcacttccggtttacgatcacttccggtttacagacacttccggtttacgctagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtgactcatgatgatgccacgtcaccaatgccacgtcaccaggtgactcatgggtgactcatgacgtgtgacatgccacgtcaccaatgccacgtcaccaggtgactcatgggtgactcatgggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 437 - DNA, 628 bp, synthetic construct
  source 1..628: mol_type = other DNA
ggggcggggtgatgacacagcaattcgggactttccacgcttgcgtgagaagagaccggaagtgaatgacacagcaatggatcccaacatggcggcgcccaacatggcggctaccaacatggcggcctccaacatggcggcaggcaacatggcggctgccaacatggcggcggatccgcttgcgtgagaagctgggactttcctaggggcggggttgggactttccacatgacacagcaatacctcgagggtgactcatgatgatgccacgtcaccaatgccacgtcaccaggtgactcatgggtgactcatgacgtgtgacatgccacgtcaccaatgccacgtcaccaggtgactcatgggtgactcatgggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 438 - DNA, 642 bp, synthetic construct
  source 1..642: mol_type = other DNA
ggggcggggtgatgacacagcaattcgggactttccacgcttgcgtgagaagagaccggaagtgaatgacacagcaatggatcctcctttgatgtacgcaactcctttgatgtctatgcgtcctttgatgttaaggattcctttgatgtaggtacatcctttgatgtccgtaaatcctttgatgtggatccgcttgcgtgagaagctgggactttcctaggggcggggttgggactttccacatgacacagcaatacctcgagggtgactcatgatgatgccacgtcaccaatgccacgtcaccaggtgactcatgggtgactcatgacgtgtgacatgccacgtcaccaatgccacgtcaccaggtgactcatgggtgactcatgggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 439 - DNA, 699 bp, synthetic construct
  source 1..699: mol_type = other DNA
taattgctgagtcattgctgctatgtaattgctgagtcatatgcctatcctaattgctgagtcataatcgagatgtaattgctgagtcatgtccgacgcataattgctgagtcattctaactcgctaattgctgagtcatgtcgacgctagcggtgactcatgatgatgccacgtcaccaatgccacgtcaccaggtgactcatgggtgactcatgacgtgtgacatgccacgtcaccaatgccacgtcaccaggtgactcatgggtgactcatgactagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 440 - DNA, 810 bp, synthetic construct
  source 1..810: mol_type = other DNA
taattgctgagtcattgctgctatgtaattgctgagtcatatgcctatcctcctttgatgtacgcaactcctttgatgtctatgcgtaattgctgagtcataatcgagatgtaattgctgagtcatgtccgacgcatcctttgatgttaaggattcctttgatgtaggtacataattgctgagtcattctaactcgctaattgctgagtcatcatctcgacctcctttgatgtccgtaaatcctttgatgtgtcgacgctagcggtgactcatgatgatgccacgtcaccaatgccacgtcaccaggtgactcatgggtgactcatgacgtgtgacatgccacgtcaccaatgccacgtcaccaggtgactcatgggtgactcatgactagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 441 - DNA, 570 bp, synthetic construct
  source 1..570: mol_type = other DNA
taattgctgagtcattgctgctatgtaattgctgagtcatatgcctatcctaattgctgagtcataatcgagatgtaattgctgagtcatgtccgacgcataattgctgagtcattctaactcgctaattgctgagtcatgtcgacactagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 442 - DNA, 816 bp, synthetic construct
  source 1..816: mol_type = other DNA
ggtgactcatgatgatgccacgtcaccaatgccacgtcaccaggtgactcatgggtgactcatgacgtgtgacatgccacgtcaccaatgccacgtcaccaggtgactcatgggtgactcatgactagtgaattctaattgctgagtcattgctgctatgtaattgctgagtcatatgcctatcctcctttgatgtacgcaactcctttgatgtctatgcgtaattgctgagtcataatcgagatgtaattgctgagtcatgtccgacgcatcctttgatgttaaggattcctttgatgtaggtacataattgctgagtcattctaactcgctaattgctgagtcatcatctcgacctcctttgatgtccgtaaatcctttgatgtgtcgacactagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 443 - DNA, 660 bp, synthetic construct
  source 1..660: mol_type = other DNA
tcctttgatgtacgcaactcctttgatgtctatgcgtcctttgatgttaaggattcctttgatgtaggtacatcctttgatgtccgtaaatcctttgatgtgtcgacgctagcggtgactcatgatgatgccacgtcaccaatgccacgtcaccaggtgactcatgggtgactcatgacgtgtgacatgccacgtcaccaatgccacgtcaccaggtgactcatgggtgactcatgactagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 444 - DNA, 669 bp, synthetic construct
  source 1..669: mol_type = other DNA
ggtgactcatgatgatgccacgtcaccaatgccacgtcaccaggtgactcatgggtgactcatgacgtgtgacatgccacgtcaccaatgccacgtcaccaggtgactcatgggtgactcatgactagtgaattcgactcctttgatgtacgcaactcctttgatgtctatgcgtcctttgatgttaaggattcctttgatgtaggtacatcctttgatgtccgtaaatcctttgatgtgtcgacactagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 445 - DNA, 439 bp, synthetic construct
  source 1..439: mol_type = other DNA
atgactcagcaattagcgagttagaatgactcagcaattatgcgtcggacatgactcagcaattacatctcgattatgactcagcaattaggataggcatatgactcagcaattacatagcagcaatgactcagcaattagctagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtaccgggaaaagttcagctgagagatataaaagagcagtctttccagcacctgcgtatcccaggaggagcaagtggcacgtcttcgggtgagtgtgcggctgtgctggagcccgggttaccagctctta

SEQ ID NO: 446 - DNA, 387 bp, synthetic construct
  source 1..387: mol_type = other DNA
atgactcagcaattagcgagttagaatgactcagcaattatgcgtcggacatgactcagcaattacatctcgattatgactcagcaattaggataggcatatgactcagcaattacatagcagcaatgactcagcaattagctagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtacctgcgctcccgacatgccccgcggcgcgccattaaccgccagatttgagtcgcgggacccgttggcagaggtgg

SEQ ID NO: 447 - DNA, 422 bp, synthetic construct
  source 1..422: mol_type = other DNA
ggtgactcatgatgatgccacgtcaccaatgccacgtcaccaggtgactcatgggtgactcatgacgtgtgacatgccacgtcaccaatgccacgtcaccaggtgactcatgggtgactcatgactagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtaccgggaaaagttcagctgagagatataaaagagcagtctttccagcacctgcgtatcccaggaggagcaagtggcacgtcttcgggtgagtgtgcggctgtgctggagcccgggttaccagctctta

SEQ ID NO: 448 - DNA, 370 bp, synthetic construct
  source 1..370: mol_type = other DNA
ggtgactcatgatgatgccacgtcaccaatgccacgtcaccaggtgactcatgggtgactcatgacgtgtgacatgccacgtcaccaatgccacgtcaccaggtgactcatgggtgactcatgactagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtacctgcgctcccgacatgccccgcggcgcgccattaaccgccagatttgagtcgcgggacccgttggcagaggtgg

SEQ ID NO: 449 - DNA, 687 bp, synthetic construct
  source 1..687: mol_type = other DNA
atgactcagcaattagcgagttagaatgactcagcaattatgcgtcggacatgactcagcaattacatctcgattatgactcagcaattaggataggcatatgactcagcaattacatagcagcaatgactcagcaattagctagtaagcttggggcggggtgatgacacagcaattcgggactttccacgcttgcgtgagaagagaccggaagtgaatgacacagcaatggatccgggaggaagtcgtaaaacttgggaggaagtcgtaaaaaatgggaggaagtcgtaaaatgcgggaggaagtcgtaaaagaagggaggaagtcgtaaaaatcgggaggaagtcgtaaaaggatccgcttgcgtgagaagctgggactttcctaggggcggggttgggactttccacatgacacagcaatacctcgagggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 450 - DNA, 671 bp, synthetic construct
  source 1..671: mol_type = other DNA
atgactcagcaattagcgagttagaatgactcagcaattatgcgtcggacatgactcagcaattacatctcgattatgactcagcaattaggataggcatatgactcagcaattacatagcagcaatgactcagcaattagctagtaagcttggggcggggtgatgacacagcaattcgggactttccacgcttgcgtgagaagagaccggaagtgaatgacacagcaatggatcctcctttgatgtacgcaactcctttgatgtctatgcgtcctttgatgttaaggattcctttgatgtaggtacatcctttgatgtccgtaaatcctttgatgtggatccgcttgcgtgagaagctgggactttcctaggggcggggttgggactttccacatgacacagcaatacctcgagggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 451 - DNA, 670 bp, synthetic construct
  source 1..670: mol_type = other DNA
ggtgactcatgatgatgccacgtcaccaatgccacgtcaccaggtgactcatgggtgactcatgacgtgtgacatgccacgtcaccaatgccacgtcaccaggtgactcatgggtgactcatgactagtaagcttggggcggggtgatgacacagcaattcgggactttccacgcttgcgtgagaagagaccggaagtgaatgacacagcaatggatccttttacgacttcctcccgatttttacgacttcctcccttcttttacgacttcctcccgcattttacgacttcctcccattttttacgacttcctcccaagttttacgacttcctcccggatccgcttgcgtgagaagctgggactttcctaggggcggggttgggactttccacatgacacagcaatacctcgagggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 452 - DNA, 654 bp, synthetic construct
  source 1..654: mol_type = other DNA
ggtgactcatgatgatgccacgtcaccaatgccacgtcaccaggtgactcatgggtgactcatgacgtgtgacatgccacgtcaccaatgccacgtcaccaggtgactcatgggtgactcatgactagtaagcttggggcggggtgatgacacagcaattcgggactttccacgcttgcgtgagaagagaccggaagtgaatgacacagcaatggatcctcctttgatgtacgcaactcctttgatgtctatgcgtcctttgatgttaaggattcctttgatgtaggtacatcctttgatgtccgtaaatcctttgatgtggatccgcttgcgtgagaagctgggactttcctaggggcggggttgggactttccacatgacacagcaatacctcgagggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 453 - DNA, 351 bp, synthetic construct
  source 1..351: mol_type = other DNA
atgactcagcaattagcgagttagaatgactcagcaattatgcgtcggacatgactcagcaattacatctcgattatgactcagcaattaggataggcatatgactcagcaattacatagcagcaatgactcagcaattagctagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtacctataaaaggccagcagcagcctgaccacatctcatcc

SEQ ID NO: 454 - DNA, 334 bp, synthetic construct
  source 1..334: mol_type = other DNA
ggtgactcatgatgatgccacgtcaccaatgccacgtcaccaggtgactcatgggtgactcatgacgtgtgacatgccacgtcaccaatgccacgtcaccaggtgactcatgggtgactcatgactagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtacctataaaaggccagcagcagcctgaccacatctcatcc

SEQ ID NO: 455 - DNA, 662 bp, synthetic construct
  source 1..662: mol_type = other DNA
atgactcagcaattagcgagttagaatgactcagcaattatgcgtcggacatgactcagcaattacatctcgattatgactcagcaattaggataggcatatgactcagcaattacatagcagcaatgactcagcaattagctagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtaccacctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcaagaagcttggaccgcatcctagccgccgactcacacaaggcaggtgggtgaggaaatccaggtaaggctcctgacagcagctttagaagggtacttgctggagtgaattcgggcctctgatt

SEQ ID NO: 456 - DNA, 645 bp, synthetic construct
  source 1..645: mol_type = other DNA
ggtgactcatgatgatgccacgtcaccaatgccacgtcaccaggtgactcatgggtgactcatgacgtgtgacatgccacgtcaccaatgccacgtcaccaggtgactcatgggtgactcatgactagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtaccacctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcaagaagcttggaccgcatcctagccgccgactcacacaaggcaggtgggtgaggaaatccaggtaaggctcctgacagcagctttagaagggtacttgctggagtgaattcgggcctctgatt

SEQ ID NO: 457 - DNA, 644 bp, synthetic construct
  source 1..644: mol_type = other DNA
taattgctgagtcattgctgctatgtaattgctgagtcatatgcctatcctaattgctgagtcataatcgagatgtaattgctgagtcatgtccgacgcataattgctgagtcattctaactcgctaattgctgagtcatgtcgacgctagcggtgactcatgatgatgccacgtcaccaatgccacgtcaccaggtgactcatgggtgactcatgacgtgtgacatgccacgtcaccaatgccacgtcaccaggtgactcatgggtgactcatgactagttcctttgatgtacgcaactcctttgatgtctatgcgtcctttgatgttaaggattcctttgatgtaggtacatcctttgatgtccgtaaatcctttgatgtctcgagggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 458 - DNA, 630 bp, synthetic construct
  source 1..630: mol_type = other DNA
taattgctgagtcattgctgctatgtaattgctgagtcatatgcctatcctaattgctgagtcataatcgagatgtaattgctgagtcatgtccgacgcataattgctgagtcattctaactcgctaattgctgagtcatgtcgacgctagcggtgactcatgatgatgccacgtcaccaatgccacgtcaccaggtgactcatgggtgactcatgacgtgtgacatgccacgtcaccaatgccacgtcaccaggtgactcatgggtgactcatgactagtcaacatggcggcgcccaacatggcggctaccaacatggcggcctccaacatggcggcaggcaacatggcggctgccaacatggcggcctcgagggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 459 - DNA, 644 bp, synthetic construct
  source 1..644: mol_type = other DNA
tcctttgatgtacgcaactcctttgatgtctatgcgtcctttgatgttaaggattcctttgatgtaggtacatcctttgatgtccgtaaatcctttgatgtgtcgacgctagcggtgactcatgatgatgccacgtcaccaatgccacgtcaccaggtgactcatgggtgactcatgacgtgtgacatgccacgtcaccaatgccacgtcaccaggtgactcatgggtgactcatgactagttaattgctgagtcattgctgctatgtaattgctgagtcatatgcctatcctaattgctgagtcataatcgagatgtaattgctgagtcatgtccgacgcataattgctgagtcattctaactcgctaattgctgagtcatctcgagggtaccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 460 - DNA, 73 bp, synthetic construct
  source 1..73: mol_type = other DNA
acgcgtcccgacatgccccgcggcgcgccattaaccgccagatttgagtcgcgggacccgttggcagaggtgg

SEQ ID NO: 461 - DNA, 635 bp, synthetic construct
  source 1..635: mol_type = other DNA
ggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtgactcatgatgatgccacgtcaccaatgccacgtcaccaggtgactcatgggtgactcatgacgtgtgacatgccacgtcaccaatgccacgtcaccaggtgactcatgggtgactcatgggtaccacctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcaaaaatccagagcggcgggcactgacgggcacttgcaccgtgtggacagactctccggttctgtgagtggtttttcttttcccgggtcggacctggagttcttagggggatggctgaagaattca

SEQ ID NO: 462 - DNA, 634 bp, synthetic construct
  source 1..634: mol_type = other DNA
ggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtgactcatgatgatgccacgtcaccaatgccacgtcaccaggtgactcatgggtgactcatgacgtgtgacatgccacgtcaccaatgccacgtcaccaggtgactcatgggtgactcatgggtaccacctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcaagaagcttggaccgcatcctagccgccgactcacacaaggcaggtgggtgaggaaatccaggtaaggctcctgacagcagctttagaagggtacttgctggagtgaattcgggcctctgatta

SEQ ID NO: 463 - DNA, 588 bp, synthetic construct
  source 1..588: mol_type = other DNA
ggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtgactcatgatgatgccacgtcaccaatgccacgtcaccaggtgactcatgggtgactcatgacgtgtgacatgccacgtcaccaatgccacgtcaccaggtgactcatgggtgactcatgggtaccacctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcaagtatcccaggaggagcaagtggcacgtcttcgggtgagtgtgcggctgtgctggagcccgggttaccagctcttaa

SEQ ID NO: 464 - DNA, 609 bp, synthetic construct
  source 1..609: mol_type = other DNA
ggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtgactcatgatgatgccacgtcaccaatgccacgtcaccaggtgactcatgggtgactcatgacgtgtgacatgccacgtcaccaatgccacgtcaccaggtgactcatgggtgactcatgggtaccacctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcaacactcgcgctgccatcactcttccgccgtcttcgccgccatcctcggcgcgactcgcttctttcggttctaccaggtagagtccgccgccatcctca

SEQ ID NO: 465 - DNA, 563 bp, synthetic construct
  source 1..563: mol_type = other DNA
ggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtgactcatgatgatgccacgtcaccaatgccacgtcaccaggtgactcatgggtgactcatgacgtgtgacatgccacgtcaccaatgccacgtcaccaggtgactcatgggtgactcatgggtaccacctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcaactttttccgtgctacctgcagaggggtccatacggcgttgttctggattca

SEQ ID NO: 466 - DNA, 716 bp, synthetic construct
  source 1..716: mol_type = other DNA
ggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtgactcatgatgatgccacgtcaccaatgccacgtcaccaggtgactcatgggtgactcatgacgtgtgacatgccacgtcaccaatgccacgtcaccaggtgactcatgggtgactcatgggtaccacctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcaacggcggcgcagatcgcccggcgcggctccgccccctgcgccggtcacgtgggggcgccggctgcgcctgcggagaagcggtggccgccgagcgggatctgtgcggggagccggaaatggttgtggactacgtctgtgcggctgcgtggggctcggccgcgcggactgaaggagactgaaggtgctggggggaccctgatgtgga

SEQ ID NO: 467 - DNA, 457 bp, synthetic construct
  source 1..457: mol_type = other DNA
ggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtgactcatgatgatgccacgtcaccaatgccacgtcaccaggtgactcatgggtgactcatgacgtgtgacatgccacgtcaccaatgccacgtcaccaggtgactcatgggtgactcatgggtaccgggaaaagttcagctgagagatataaaagagcagtctttccagcacctgcgaagcttggaccgcatcctagccgccgactcacacaaggcaggtgggtgaggaaatccaggtaaggctcctgacagcagctttagaagggtacttgctggagtgaattcgggcctctgatta

SEQ ID NO: 468 - DNA, 411 bp, synthetic construct
  source 1..411: mol_type = other DNA
ggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtgactcatgatgatgccacgtcaccaatgccacgtcaccaggtgactcatgggtgactcatgacgtgtgacatgccacgtcaccaatgccacgtcaccaggtgactcatgggtgactcatgggtaccgggaaaagttcagctgagagatataaaagagcagtctttccagcacctgcgtatcccaggaggagcaagtggcacgtcttcgggtgagtgtgcggctgtgctggagcccgggttaccagctcttaa

SEQ ID NO: 469 - DNA, 432 bp, synthetic construct
  source 1..432: mol_type = other DNA
ggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtgactcatgatgatgccacgtcaccaatgccacgtcaccaggtgactcatgggtgactcatgacgtgtgacatgccacgtcaccaatgccacgtcaccaggtgactcatgggtgactcatgggtaccgggaaaagttcagctgagagatataaaagagcagtctttccagcacctgccactcgcgctgccatcactcttccgccgtcttcgccgccatcctcggcgcgactcgcttctttcggttctaccaggtagagtccgccgccatcctca

SEQ ID NO: 470 - DNA, 385 bp, synthetic construct
  source 1..385: mol_type = other DNA
ggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtgactcatgatgatgccacgtcaccaatgccacgtcaccaggtgactcatgggtgactcatgacgtgtgacatgccacgtcaccaatgccacgtcaccaggtgactcatgggtgactcatgggtaccgggaaaagttcagctgagagatataaaagagcagtctttccagcacctgcctttttccgtgctacctgcagaggggtccatacggcgttgttctggattc

SEQ ID NO: 471 - DNA, 538 bp, synthetic construct
  source 1..538: mol_type = other DNA
ggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtgactcatgatgatgccacgtcaccaatgccacgtcaccaggtgactcatgggtgactcatgacgtgtgacatgccacgtcaccaatgccacgtcaccaggtgactcatgggtgactcatgggtaccgggaaaagttcagctgagagatataaaagagcagtctttccagcacctgccggcggcgcagatcgcccggcgcggctccgccccctgcgccggtcacgtgggggcgccggctgcgcctgcggagaagcggtggccgccgagcgggatctgtgcggggagccggaaatggttgtggactacgtctgtgcggctgcgtggggctcggccgcgcggactgaaggagactgaaggtgctggggggaccctgatgtgg

SEQ ID NO: 472 - DNA, 584 bp, synthetic construct
  source 1..584: mol_type = other DNA
ggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtgactcatgatgatgccacgtcaccaatgccacgtcaccaggtgactcatgggtgactcatgacgtgtgacatgccacgtcaccaatgccacgtcaccaggtgactcatgggtgactcatgggtacccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccaaaatccagagcggcgggcactgacgggcacttgcaccgtgtggacagactctccggttctgtgagtggtttttcttttcccgggtcggacctggagttcttagggggatggctgaagaattc

SEQ ID NO: 473 - DNA, 583 bp, synthetic construct
  source 1..583: mol_type = other DNA
ggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtgactcatgatgatgccacgtcaccaatgccacgtcaccaggtgactcatgggtgactcatgacgtgtgacatgccacgtcaccaatgccacgtcaccaggtgactcatgggtgactcatgggtacccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagaagcttggaccgcatcctagccgccgactcacacaaggcaggtgggtgaggaaatccaggtaaggctcctgacagcagctttagaagggtacttgctggagtgaattcgggcctctgatt

SEQ ID NO: 474 - DNA, 558 bp, synthetic construct
  source 1..558: mol_type = other DNA
ggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtgactcatgatgatgccacgtcaccaatgccacgtcaccaggtgactcatgggtgactcatgacgtgtgacatgccacgtcaccaatgccacgtcaccaggtgactcatgggtgactcatgggtacccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccacactcgcgctgccatcactcttccgccgtcttcgccgccatcctcggcgcgactcgcttctttcggttctaccaggtagagtccgccgccatcctc

SEQ ID NO: 475 - DNA, 512 bp, synthetic construct
  source 1..512: mol_type = other DNA
ggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtgactcatgatgatgccacgtcaccaatgccacgtcaccaggtgactcatgggtgactcatgacgtgtgacatgccacgtcaccaatgccacgtcaccaggtgactcatgggtgactcatgggtacccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccactttttccgtgctacctgcagaggggtccatacggcgttgttctggattc

SEQ ID NO: 476 - DNA, 665 bp, synthetic construct
  source 1..665: mol_type = other DNA
ggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtgactcatgatgatgccacgtcaccaatgccacgtcaccaggtgactcatgggtgactcatgacgtgtgacatgccacgtcaccaatgccacgtcaccaggtgactcatgggtgactcatgggtacccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccacggcggcgcagatcgcccggcgcggctccgccccctgcgccggtcacgtgggggcgccggctgcgcctgcggagaagcggtggccgccgagcgggatctgtgcggggagccggaaatggttgtggactacgtctgtgcggctgcgtggggctcggccgcgcggactgaaggagactgaaggtgctggggggaccctgatgtgg

SEQ ID NO: 477 - DNA, 183 bp, synthetic construct
  source 1..183: mol_type = other DNA
agtatagtgcacagtgactgcagcagggtgactcatgatgatgccacgtcaccaatgccacgtcaccaggtgactcatgggtgactcatgatgccacgtcaccaatgccacgtcaccaggtgactcatgggtgactcatgggtacctataaaaggccagcagcagcctgaccacatctcatcc

SEQ ID NO: 478 - DNA, 439 bp, synthetic construct
  source 1..439: mol_type = other DNA
tcctttgatgtacgcaactcctttgatgtctatgcgtcctttgatgttaaggattcctttgatgtaggtacatcctttgatgtccgtaaatcctttgatgtaagcttaactcgcaatctagcatcgtccgacgcaacgccttacaccatcagaatctgctagcggtgactcatgggtgactcatgggtgactcatgggtgactcatgctacgtggtgactcatgggtgactcatgggtgactcatgggtgactcatgggtgactcatgggtaccgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctga

SEQ ID NO: 479 - DNA, 464 bp, synthetic construct
  source 1..464: mol_type = other DNA
tcctttgatgtacgcaactcctttgatgtctatgcgtcctttgatgttaaggattcctttgatgtaggtacatcctttgatgtccgtaaatcctttgatgtaagcttggtacaacttctcacggaggcttctaactcgcaatctagcatcgtccgacgcaacgccttacaccatcagaatctgctagcggtgactcatgggtgactcatgggtgactcatgggtgactcatgctacgtggtgactcatgggtgactcatgggtgactcatgggtgactcatgggtgactcatgggtaccgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctga

SEQ ID NO: 480 - DNA, 596 bp, synthetic construct
  source 1..596: mol_type = other DNA
tcctttgatgtacgcaactcctttgatgtctatgcgtcctttgatgttaaggattcctttgatgtaggtacatcctttgatgtccgtaaatcctttgatgtgacgattcttgatatcctcgaggctagcatgatcaccatgagtcacccatgagtcacccatgagtcacccatgagtcacccatgagtcacccatgagtcacccatgagtcacccatgagtcacccatgagtcaccactagtggtaccacctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgatta

SEQ ID NO: 481 - DNA, 641 bp, synthetic construct
  source 1..641: mol_type = other DNA
tcctttgatgtacgcaactcctttgatgtctatgcgtcctttgatgttaaggattcctttgatgtaggtacatcctttgatgtccgtaaatcctttgatgtgacgatcttgatatcctcgaggctagcatgatcaccatgagtcacccatgagtcacccatgagtcacccatgagtcacccatgagtcacccatgagtcacccatgagtcacccatgagtcacccatgagtcaccactagtggtaccacctcttaacaatacgtttcacaaatagttaaaaacatgcatactagtggggcggggtgatgacacagcaattcgggactttccacgcttgcgtgagaagagaccggaagtgaatgacacagcaattcgcttgcgtgagaagctgggactttcctaggggcggggttgggactttccacatgacacagcaatacactagtaacatttctctggcctaactggccggtaccgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctg

SEQ ID NO: 482 - DNA, 643 bp, synthetic construct
  source 1..643: mol_type = other DNA
tcctttgatgtacgcaactcctttgatgtctatgcgtcctttgatgttaaggattcctttgatgtaggtacatcctttgatgtccgtaaatcctttgatgtgacgatcttgatatcctcgaggctagcatgatcaccatgagtcacccatgagtcacccatgagtcacccatgagtcacccatgagtcacccatgagtcacccatgagtcacccatgagtcacccatgagtcaccactagtggtaccacctcttaacaatacgtttcacaaatagttaaaaacatgcatactagtggggcggggtgatgacacagcaattcgggactttccacgcttgcgtgagaagagaccggaagtgaatgacacagcaattcgcttgcgtgagaagctgggactttcctaggggcggggttgggactttccacatgacacagcaatacactagtaacatttctctggcctaactggccggtaccgggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctgaa

SEQ ID NO: 483 - DNA, 1090 bp, synthetic construct
  source 1..1090: mol_type = other DNA
atgactcagcaattagcgagttagaatgactcagcaattatgcgtcggacatgactcagcaattacatctcgattatgactcagcaattaggataggcatatgactcagcaattacatagcagcaatgactcagcaattagctagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtaccatgcatactagtctgagcgacagtatagtgcacagtgactgcagcagtcattcctttgatgtacgcaactcctttgatgtctatgcgtcctttgatgttaaggattcctttgatgtaggtacatcctttgatgtccgtaaatcctttgatgtgacgtctacgtatctacctgatcaaacatgcccggacatgtcgtaagacataaacatgcccggacatgtcctcgcaatctaacatgcccggacatgtcctcgcaatctaacatgcccggacatgtctgcaagctacaacatgcccggacatgtctacaatatacgtatctacctgatcaaacatgcccggacatgtcgtaagacataaacatgcccggacatgtcctcgcaatctaacatgcccggacatgtcctcgcaatctaacatgcccggacatgtctgcaagctacaacatgcccggacatgtctacgtacatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgatta

SEQ ID NO: 484 - DNA, 616 bp, synthetic construct
  source 1..616: mol_type = other DNA
atgactcagcaattagcgagttagaatgactcagcaattatgcgtcggacatgactcagcaattacatctcgattatgactcagcaattaggataggcatatgactcagcaattacatagcagcaatgactcagcaattagctagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtaccactagtgtcatctctttgaatattctgtagtttgaggagaatatttgttatattgcacaataaaataagtttgcaagttttttttttctgccccaaagagctctgtgtccttgaacataaaatacaaataaccgctatgctgttaattattaacaaatgtcccattttcaacctaaggaaataccataaagtaacagatataccaacaaaaggttaataattaacaggcattgcctgaaaagagtataaaaggctttcagcatgattttccatattgtgcttccaccactgccaataacaaa

SEQ ID NO: 485 - DNA, 458 bp, synthetic construct
  source 1..458: mol_type = other DNA
atgactcagcaattagcgagttagaatgactcagcaattatgcgtcggacatgactcagcaattacatctcgattatgactcagcaattaggataggcatatgactcagcaattacatagcagcaatgactcagcaattagctagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtaccagcttgcatgcctgcaggtcggagtactgtcctccgagcggagtactgtcctccgagcggagtactgtcctccgagcggagtactgtcctccgagcggagtactgtcctccgagcggagactctagagggtatataatggatcc

SEQ ID NO: 486 - DNA, 279 bp, synthetic construct
  source 1..279: mol_type = other DNA
tctgtagtttgaggagaatatttgttatattgcacaataaaataagtttgcaagttttttttttctgccccaaagagctctgtgtccttgaacataaaatacaaataaccgctatgctgttaattattaacaaatgtcccattttcaacctaaggaaataccataaagtaacagatataccaacaaaaggttaataattaacaggcattgcctgaaaagagtataaaaggctttcagcatgattttccatattgtgcttccaccactgccaataacaaa

SEQ ID NO: 487 - DNA, 14 bp, synthetic construct
  source 1..14: mol_type = other DNA
ttttcccgccaaaa

SEQ ID NO: 488 - DNA, 10 bp, synthetic construct
  source 1..10: mol_type = other DNA
tgatcaataa

SEQ ID NO: 489 - DNA, 11 bp, synthetic construct
  source 1..11: mol_type = other DNA
gaaacatgagc

SEQ ID NO: 490 - DNA, 12 bp, synthetic construct
  source 1..12: mol_type = other DNA
agacatcaaagg

SEQ ID NO: 491 - DNA, 12 bp, synthetic construct
  source 1..12: mol_type = other DNA
aaacttcaaagg

SEQ ID NO: 492 - DNA, 11 bp, synthetic construct
  source 1..11: mol_type = other DNA
tcctttgatct

SEQ ID NO: 493 - DNA, 10 bp, synthetic construct
  source 1..10: mol_type = other DNA
gtcgtaaact

SEQ ID NO: 494 - DNA, 10 bp, synthetic construct
  source 1..10: mol_type = other DNA
gtcgtaaagt

SEQ ID NO: 495 - DNA, 10 bp, synthetic construct
  source 1..10: mol_type = other DNA
gtcgtaaatt

SEQ ID NO: 496 - DNA, 12 bp, synthetic construct
  source 1..12: mol_type = other DNA
gtccacgtggcc

SEQ ID NO: 497 - DNA, 13 bp, synthetic construct
  source 1..13: mol_type = other DNA
ggccccgcccacc

SEQ ID NO: 498 - DNA, 11 bp, synthetic construct
  source 1..11: mol_type = other DNA
tcctttgaagt

SEQ ID NO: 499 - DNA, 10 bp, synthetic construct
  source 1..10: mol_type = other DNA
agcggaagtg

SEQ ID NO: 500 - DNA, 12 bp, synthetic construct
  source 1..12: mol_type = other DNA
gggggaagggag

SEQ ID NO: 501 - DNA, 10 bp, synthetic construct
  source 1..10: mol_type = other DNA
gtcgtaaaat

SEQ ID NO: 502 - DNA, 15 bp, synthetic construct
  source 1..15: mol_type = other DNA
gtcatgcatgactgc

SEQ ID NO: 503 - DNA, 16 bp, synthetic construct
  source 1..16: mol_type = other DNA
gtttgggcgccatttc

SEQ ID NO: 504 - DNA, 13 bp, synthetic construct
  source 1..13: mol_type = other DNA
ggacccgcccacc

SEQ ID NO: 505 - DNA, 11 bp, synthetic construct
  source 1..11: mol_type = other DNA
gaaacctgagc

SEQ ID NO: 506 - DNA, 11 bp, synthetic construct
  source 1..11: mol_type = other DNA
gaaacttgagc

SEQ ID NO: 507 - DNA, 15 bp, synthetic construct
  source 1..15: mol_type = other DNA
attaatcgattattt

SEQ ID NO: 508 - DNA, 11 bp, synthetic construct
  source 1..11: mol_type = other DNA
gtctgtggctt

SEQ ID NO: 509 - DNA, 20 bp, synthetic construct
  source 1..20: mol_type = other DNA
ccccaaaccaccaccccccc

SEQ ID NO: 510 - DNA, 12 bp, synthetic construct
  source 1..12: mol_type = other DNA
aaagatcaaagg

SEQ ID NO: 511 - DNA, 21 bp, synthetic construct
  source 1..21: mol_type = other DNA
ataaatataaaaggactaatt

SEQ ID NO: 512 - DNA, 12 bp, synthetic construct
  source 1..12: mol_type = other DNA
agagatcaaagg

SEQ ID NO: 513 - DNA, 12 bp, synthetic construct
  source 1..12: mol_type = other DNA
ttgaaaaaaaaa

SEQ ID NO: 514 - DNA, 15 bp, synthetic construct
  source 1..15: mol_type = other DNA
agttaattattaact

SEQ ID NO: 515 - DNA, 15 bp, synthetic construct
  source 1..15: mol_type = other DNA
attaatcaattattt

SEQ ID NO: 516 - DNA, 13 bp, synthetic construct
  source 1..13: mol_type = other DNA
ggccccgcctacc

SEQ ID NO: 517 - DNA, 16 bp, synthetic construct
  source 1..16: mol_type = other DNA
attttggcgcgaaaat

SEQ ID NO: 518 - DNA, 11 bp, synthetic construct
  source 1..11: mol_type = other DNA
aattaggtcac

SEQ ID NO: 519 - DNA, 15 bp, synthetic construct
  source 1..15: mol_type = other DNA
attaatcaatttttt

SEQ ID NO: 520 - DNA, 13 bp, synthetic construct
  source 1..13: mol_type = other DNA
ggacacgcccacc

SEQ ID NO: 521 - DNA, 10 bp, synthetic construct
  source 1..10: mol_type = other DNA
accggaaggg

SEQ ID NO: 522 - DNA, 18 bp, synthetic construct
  source 1..18: mol_type = other DNA
aacatgcctgggcatgtc

SEQ ID NO: 523 - DNA, 18 bp, synthetic construct
  source 1..18: mol_type = other DNA
aacatgcccggacatgtc

SEQ ID NO: 524 - DNA, 12 bp, synthetic construct
  source 1..12: mol_type = other DNA
gcccacgtggcc

SEQ ID NO: 525 - DNA, 18 bp, synthetic construct
  source 1..18: mol_type = other DNA
aacatgcccgggcatgtc

SEQ ID NO: 526 - DNA, 18 bp, synthetic construct
  source 1..18: mol_type = other DNA
aacatgtccgggcatgtc

SEQ ID NO: 527 - DNA, 18 bp, synthetic construct
  source 1..18: mol_type = other DNA
aacatgttgggacatgtc

SEQ ID NO: 528 - DNA, 12 bp, synthetic construct
  source 1..12: mol_type = other DNA
gagaacaaagca

SEQ ID NO: 529 - DNA, 14 bp, synthetic construct
  source 1..14: mol_type = other DNA
atgccacgtcatca

SEQ ID NO: 530 - DNA, 23 bp, synthetic construct
  source 1..23: mol_type = other DNA
gggcgtgcgctcccgacaagccc

SEQ ID NO: 531 - DNA, 18 bp, synthetic construct
  source 1..18: mol_type = other DNA
aacatgcccaggcatgtc

SEQ ID NO: 532 - DNA, 14 bp, synthetic construct
  source 1..14: mol_type = other DNA
cctaaataaacaaa

SEQ ID NO: 533 - DNA, 15 bp, synthetic construct
  source 1..15: mol_type = other DNA
aaccgttaaacggtc

SEQ ID NO: 534 - DNA, 18 bp, synthetic construct
  source 1..18: mol_type = other DNA
aacatgtccggacatgtc

SEQ ID NO: 535 - DNA, 10 bp, synthetic construct
  source 1..10: mol_type = other DNA
ggtaattgac

SEQ ID NO: 536 - DNA, 14 bp, synthetic construct
  source 1..14: mol_type = other DNA
ttttggcgcctttt

SEQ ID NO: 537 - DNA, 16 bp, synthetic construct
  source 1..16: mol_type = other DNA
gttttggcgccttttc

SEQ ID NO: 538 - DNA, 11 bp, synthetic construct
  source 1..11: mol_type = other DNA
gaaatttgagc

SEQ ID NO: 539 - DNA, 23 bp, synthetic construct
  source 1..23: mol_type = other DNA
gggcaagcgctcccgacatgccc

SEQ ID NO: 540 - DNA, 14 bp, synthetic construct
  source 1..14: mol_type = other DNA
gaggtcaaaggtca

SEQ ID NO: 541 - DNA, 23 bp, synthetic construct
  source 1..23: mol_type = other DNA
gggctagcgctcccgacatgccc

SEQ ID NO: 542 - DNA, 11 bp, synthetic construct
  source 1..11: mol_type = other DNA
gggcgggaagg

SEQ ID NO: 543 - DNA, 11 bp, synthetic construct
  source 1..11: mol_type = other DNA
gggcgggacgg

SEQ ID NO: 544 - DNA, 12 bp, synthetic construct
  source 1..12: mol_type = other DNA
ggacacgtgccc

SEQ ID NO: 545 - DNA, 12 bp, synthetic construct
  source 1..12: mol_type = other DNA
gggaacaaagaa

SEQ ID NO: 546 - DNA, 23 bp, synthetic construct
  source 1..23: mol_type = other DNA
gggcatgcgctcccgacatgccc

SEQ ID NO: 547 - DNA, 14 bp, synthetic construct
  source 1..14: mol_type = other DNA
atgccacgtcagca

SEQ ID NO: 548 - DNA, 13 bp, synthetic construct
  source 1..13: mol_type = other DNA
ggccacgcccacc

SEQ ID NO: 549 - DNA, 20 bp, synthetic construct
  source 1..20: mol_type = other DNA
ccccaaaacaaccccccccc

SEQ ID NO: 550 - DNA, 12 bp, synthetic construct
  source 1..12: mol_type = other DNA
ggccacgtggcc

SEQ ID NO: 551 - DNA, 116 bp, synthetic construct
  source 1..116: mol_type = other DNA
ccttgccgaaaagatttgtctgaggaactgaaaatagaagggaaaaaagaggagggacaaaagaggcagaaatgagaggggaggggacagaggacacctgaataaagaccacaccc

SEQ ID NO: 552 - DNA, 66 bp, synthetic construct
  source 1..66: mol_type = other DNA
gacccacgtgatgctgagaagtactcctgccctaggaagagactcagggcagagggaggaaggaca

SEQ ID NO: 553 - DNA, 69 bp, Homo sapiens
  source 1..69: mol_type = genomic DNA
gctgttaattattggcaaatnnnnnnnnnnnnnnnnnnnnnnnnnnnnnnnnnnaggttactagttaac

SEQ ID NO: 554 - DNA, 69 bp, synthetic construct
  source 1..69: mol_type = other DNA
gctgttaattattaacaaatnnnnnnnnnnnnnnnnnnnnnnnnnnnnnnnnnnaggttaataattaac

SEQ ID NO: 555 - DNA, 74 bp, synthetic construct
  source 1..74: mol_type = other DNA
ctgcgctcccgacatgccccgcggcgcgccattaaccgccagatttgagtcgcgggacccgttggcagaggtgg

SEQ ID NO: 556 - DNA, 581 bp, synthetic construct
  source 1..581: mol_type = other DNA
ggtgactcatgatgatgccacgtcaccaatgccacgtcaccaggtgactcatgggtgactcatgacgtgtgacatgccacgtcaccaatgccacgtcaccaggtgactcatgggtgactcatgactagtgaattctaattgctgagtcattgctgctatgtaattgctgagtcatatgcctatcctaattgctgagtcataatcgagatgtaattgctgagtcatgtccgacgcataattgctgagtcattctaactcgctaattgctgagtcatgtcgacactagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtaccgggaaaagttcagctgagagatataaaagagcagtctttccagcacctgcgtatcccaggaggagcaagtggcacgtcttcgggtgagtgtgcggctgtgctggagcccgggttaccagctcttaa

SEQ ID NO: 557 - DNA, 705 bp, synthetic construct
  source 1..705: mol_type = other DNA
ggtgactcatgatgatgccacgtcaccaatgccacgtcaccaggtgactcatgggtgactcatgacgtgtgacatgccacgtcaccaatgccacgtcaccaggtgactcatgggtgactcatgactagtgaattctaattgctgagtcattgctgctatgtaattgctgagtcatatgcctatcctaattgctgagtcataatcgagatgtaattgctgagtcatgtccgacgcataattgctgagtcattctaactcgctaattgctgagtcatgtcgacactagtaagcttggggcggggtgatgacacagcaattcgggactttccacgcttgcgtgagaagagaccggaagtgaatgacacagcaatggatccgcttgcgtgagaagctgggactttcctaggggcggggttgggactttccacatgacacagcaatacctcgagggtacggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgcagtgtgcggctgtgctggagcccgggttaccagctctt

SEQ ID NO: 558 - DNA, 309 bp, synthetic construct
  source 1..309: mol_type = other DNA
catactgaaaagcatacttttgcaatgttatttttaaaaacaaggaactctttaacccagggaagataatcacttggggaaaggaaggttcgtttctgagttagcaacaagtaaatgcagcactagtgggtgggattgaggtatgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtgaattcgggcctctgatta

SEQ ID NO: 559 - DNA, 348 bp, synthetic construct
  source 1..348: mol_type = other DNA
acctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcaagaagcttggaccgcatcctagccgccgactcacacaaggcaggtgggtgaggaaatccaggtaaggctcctgacagcagctttagaagggtacttgctggagtgaattcgggcctctgatt

SEQ ID NO: 560 - DNA, 382 bp, synthetic construct
  source 1..382: mol_type = other DNA
ccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccacggcggcgcagatcgcccggcgcggctccgccccctgcgccggtcacgtgggggcgccggctgcgcctgcggagaagcggtggccgccgagcgggatctgtgcggggagccggaaatggttgtggactacgtctgtgcggctgcgtggggctcggccgcgcggactgaaggagactgaaggtgctggggggaccctgatgtgga

SEQ ID NO: 561 - DNA, 235 bp, synthetic construct
  source 1..235: mol_type = other DNA
ccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccactttttccgtgctacctgcagaggggtccatacggcgttgttctggattcaccggta

SEQ ID NO: 562 - DNA, 275 bp, synthetic construct
  source 1..275: mol_type = other DNA
ccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccacactcgcgctgccatcactcttccgccgtcttcgccgccatcctcggcgcgactcgcttctttcggttctaccaggtagagtccgccgccatcctca

SEQ ID NO: 563 - DNA, 300 bp, synthetic construct
  source 1..300: mol_type = other DNA
ccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagaagcttggaccgcatcctagccgccgactcacacaaggcaggtgggtgaggaaatccaggtaaggctcctgacagcagctttagaagggtacttgctggagtgaattcgggcctctgatta

SEQ ID NO: 564 - DNA, 301 bp, synthetic construct
  source 1..301: mol_type = other DNA
ccggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccaaaatccagagcggcgggcactgacgggcacttgcaccgtgtggacagactctccggttctgtgagtggtttttcttttcccgggtcggacctggagttcttagggggatggctgaagaattca

SEQ ID NO: 565 - DNA, 432 bp, synthetic construct
  source 1..432: mol_type = other DNA
cacctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcaacggcggcgcagatcgcccggcgcggctccgccccctgcgccggtcacgtgggggcgccggctgcgcctgcggagaagcggtggccgccgagcgggatctgtgcggggagccggaaatggttgtggactacgtctgtgcggctgcgtggggctcggccgcgcggactgaaggagactgaaggtgctggggggaccctgatgtgga

SEQ ID NO: 566 - DNA, 279 bp, synthetic construct
  source 1..279: mol_type = other DNA
cacctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcaactttttccgtgctacctgcagaggggtccatacggcgttgttctggattca

SEQ ID NO: 567 - DNA, 325 bp, synthetic construct
  source 1..325: mol_type = other DNA
cacctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcaacactcgcgctgccatcactcttccgccgtcttcgccgccatcctcggcgcgactcgcttctttcggttctaccaggtagagtccgccgccatcctca

SEQ ID NO: 568 - DNA, 304 bp, synthetic construct
  source 1..304: mol_type = other DNA
cacctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcaagtatcccaggaggagcaagtggcacgtcttcgggtgagtgtgcggctgtgctggagcccgggttaccagctcttaa

SEQ ID NO: 569 - DNA, 351 bp, synthetic construct
  source 1..351: mol_type = other DNA
cacctcttaacaatacgtttcacaaatagttaaaaacatgcatactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcaaaaatccagagcggcgggcactgacgggcacttgcaccgtgtggacagactctccggttctgtgagtggtttttcttttcccgggtcggacctggagttcttagggggatggctgaagaattca

SEQ ID NO: 570 - DNA, 255 bp, synthetic construct
  source 1..255: mol_type = other DNA
cgggaaaagttcagctgagagatataaaagagcagtctttccagcacctgccggcggcgcagatcgcccggcgcggctccgccccctgcgccggtcacgtgggggcgccggctgcgcctgcggagaagcggtggccgccgagcgggatctgtgcggggagccggaaatggttgtggactacgtctgtgcggctgcgtggggctcggccgcgcggactgaaggagactgaaggtgctggggggaccctgatgtgga

SEQ ID NO: 571 - DNA, 102 bp, synthetic construct
  source 1..102: mol_type = other DNA
cgggaaaagttcagctgagagatataaaagagcagtctttccagcacctgcctttttccgtgctacctgcagaggggtccatacggcgttgttctggattca

SEQ ID NO: 572 - DNA, 148 bp, synthetic construct
  source 1..148: mol_type = other DNA
cgggaaaagttcagctgagagatataaaagagcagtctttccagcacctgccactcgcgctgccatcactcttccgccgtcttcgccgccatcctcggcgcgactcgcttctttcggttctaccaggtagagtccgccgccatcctca

SEQ ID NO: 573 - DNA, 127 bp, synthetic construct
  source 1..127: mol_type = other DNA
cgggaaaagttcagctgagagatataaaagagcagtctttccagcacctgcgtatcccaggaggagcaagtggcacgtcttcgggtgagtgtgcggctgtgctggagcccgggttaccagctcttaa

SEQ ID NO: 574 - DNA, 173 bp, synthetic construct
  source 1..173: mol_type = other DNA
cgggaaaagttcagctgagagatataaaagagcagtctttccagcacctgcgaagcttggaccgcatcctagccgccgactcacacaaggcaggtgggtgaggaaatccaggtaaggctcctgacagcagctttagaagggtacttgctggagtgaattcgggcctctgatta

SEQ ID NO: 575 - DNA, 191 bp, synthetic construct; identical to SEQ ID NO: 399
  source 1..191: mol_type = other DNA

SEQ ID NO: 576 - DNA, 191 bp, synthetic construct
  source 1..191: mol_type = other DNA
agtggtgggggagtgaaaagagagatggagaaagaggggatgggcagaaagaggaggaggagtcaggggcagggcatggaggtgggtggggctgggctgccaaagcaggataaatgcacacctgcctgctggtctgggctccctgcctcgggctctcaccctcctctcctgcagctccagctttgtgctct

SEQ ID NO: 577 - DNA, 291 bp, synthetic construct
  source 1..291: mol_type = other DNA
catactgaaaagcatacttttgcaatgttatttttaaaaacaaggaactctttaacccagggaagataatcacttggggaaaggaaggttcgtttctgagttagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccaggtaaggctcctgacagcagctttagaagggtacttgctggagtg

SEQ ID NO: 578 - DNA, 250 bp, synthetic construct
  source 1..250: mol_type = other DNA
ggcccgccccctttccttacgcggattggtagctgcaggcttccctatctgattggccgaacgaacgcagcgcgtaatttaaaatattgtatctgtaacaaagctgcacctcgtgggcggagttgtgctctgcggctgcgaaagtccagcttcggcgactaggtgtgagtaagccagtatcccaggaggagcaagtggcacgtcttcgggtgagtgtgcggctgtgctggagcccgggttaccagctctt

SEQ ID NO: 579 - DNA, 165 bp, synthetic construct
  source 1..165: mol_type = other DNA
gggaaaagttcagctgagagatataaaagagcagtctttccagcacctgcaaatccagagcggcgggcactgacgggcacttgcaccgtgtggacagactctccggttctgtgagtggtttttcttttcccgggtcggacctggagttcttagggggatggctga

SEQ ID NO: 580 - DNA, 110 bp, synthetic construct
  source 1..110: mol_type = other DNA
acccacgtgatgctgagaagtactcctgccctaggaagagactcagggcagagggaggaaggacagcagaccagacagtcacagcagccttgacaaaacgttcctggaac

SEQ ID NO: 581 - DNA, 37 bp, synthetic construct
  source 1..37: mol_type = other DNA
tataaaaggccagcagcagcctgaccacatctcatcc

SEQ ID NO: 582 - DNA, 125 bp, synthetic construct
  source 1..125: mol_type = other DNA
cactcccagaaggcagcgggcgagggcgtggggccggggctctcccggcatgctctgcggcgcgcctccgcccgcgcgatttgaatcctgcgtttgagtcgtcttggcggaggttgtggtgacgc

SEQ ID NO: 583 - DNA, 67 bp, synthetic construct
  source 1..67: mol_type = other DNA
tcccgacatgccccgcggcgcgccattaaccgccagatttgagtcgcgggacccgttggcagaggtg

SEQ ID NO: 584 - DNA, 35 bp, synthetic construct
  source 1..35: mol_type = other DNA
gtatcccaggaggagcaagtggcacgtcttcgggt

SEQ ID NO: 585 - DNA, 51 bp, synthetic construct
  source 1..51: mol_type = other DNA
cgggaaaagttcagctgagagatataaaagagcagtctttccagcacctgc

SEQ ID NO: 586 - DNA, 76 bp, synthetic construct
  source 1..76: mol_type = other DNA
gtatcccaggaggagcaagtggcacgtcttcgggtgagtgtgcggctgtgctggagcccgggttaccagctcttaa

SEQ ID NO: 587 - DNA, 39 bp, synthetic construct
  source 1..39: mol_type = other DNA
cagtgtgcggctgtgctggagcccgggttaccagctctt

(19) United States
(12) Patent Application Publication
(10) Pub. No.: US 2025/0161496 A1
(43) Pub. Date: May 22, 2025

(54) SYNTHETIC CANCER-SPECIFIC PROMOTERS

(71) Applicant: EARLI Inc., Redwood City, CA (US)

(72) Inventors: Dariusz WODZIAK, Redwood City, CA (US); Shireen RUDINA, Redwood City, CA (US); Maggie C. LOUIE, Redwood City, CA (US); Yue ZHANG, Redwood City, CA (US); Elizabeth STROEBLE, Redwood City, CA (US); Albert PARK, Redwood City, CA (US); David SUHY, Redwood City, CA (US)

(21) Appl. No.: 19/034,456

(22) Filed: Jan. 22, 2025

Related U.S. Application Data
(63) Continuation-in-part of application No. PCT/US24/38613, filed on Jul. 18, 2024.
(60) Provisional application No. 63/514,317, filed on Jul. 18, 2023.
(60) Provisional application No. 63/532,316, filed on Aug. 11, 2023.

(51) Int. Cl. A61K 48/00; A61K 9/51; A61P 35/00; C12N 15/85; C12N 15/88

(52) U.S. Cl. CPC A61K 48/0058; A61K 9/5123; A61P 35/00; C12N 15/85; C12N 15/88; C12N 2800/107; C12N 2830/008

(57) ABSTRACT

Described herein are synthetic promoters and/or enhancers that are specific for cancer cells and methods of engineering synthetic cancer-specific promoters.

### CROSS REFERENCE

This application is a continuation-in-part of PCT Application No. PCT/US2024/038613, filed on Jul. 18, 2024, which claims the benefit of U.S. Provisional Application No. 63/514,317, filed on Jul. 18, 2023 and U.S. Provisional Application No. 63/532,316, filed on Aug. 11, 2023, each of which is incorporated by reference herein in its entirety.

### SEQUENCE LISTING

The instant application contains a Sequence Listing which has been submitted electronically in XML format and is hereby incorporated by reference in its entirety. Said XML format sequence listing, created on Jan. 17, 2025, is named 53531_715_501_SL.xml, and is 704,542 bytes in size.

### BACKGROUND

Endogenous cancer-activated promoters are controlled by a wide network of transcription factors (TFs), which can lead to non-ideal basal activity in non-target cells. It is also difficult to reliably predict the activity in a wide variety of cancer models.

### SUMMARY

There is a need to develop synthetic cancer-specific promoters with high specificity and sensitivity, for use in delivering polypeptides to cancer cells.

In some aspects, provided herein is a recombinant polynucleotide comprising: (a) a core promoter comprising a transcription start site (TSS), wherein the core promoter is derived from one or cancer-responsive genes that are either expressed at a higher level or are more active in cancer cells compared to non-cancer cells and operably linked to an open reading frame (ORF) and (b) a plurality of binding sites for one or more transcription factors (TFs), wherein said one or more TFs are expressed at higher levels or more active in cancer cells compared to non-cancer cells. In some embodiments, the recombinant polynucleotide further comprises a plurality of enhancers. In some embodiments, said plurality of enhancers are derived from one or more cancer-responsive genes that are either expressed at a higher level or are more active in cancer cells compared to non-cancer cells. In some embodiments, said plurality of enhancers are derived from two or more cancer-responsive genes that are either expressed at a higher level or are more active in cancer cells compared to non-cancer cells, wherein one of said plurality of enhancers comprises: (i) a transcription regulatory element with at least 90% sequence homology to an enhancer consensus sequence of two or more homologous cancer-responsive genes, and/or (ii) a sequence capable of binding a transcription associated protein as determined by chromatin immunoprecipitation (ChIP) or an in vitro transfection reporter assay.

In some aspects, provided herein is a recombinant polynucleotide comprising: (a) a core promoter comprising a transcription start site (TSS) and two or more promoter elements derived from two or more cancer-responsive genes that are either expressed at a higher level or are more active in cancer cells compared to non-cancer cells and operably linked to an open reading frame (ORF) and (b) a plurality of binding sites for one or more transcription factors (TFs), wherein said one or more TFs are expressed at higher levels or more active in cancer cells compared to non-cancer cells. In some embodiments, the recombinant polynucleotide further comprises a plurality of enhancers. In some embodiments, said plurality of enhancers are derived from one or more cancer-responsive genes that are either expressed at a higher level or are more active in cancer cells compared to non-cancer cells. In some embodiments, said plurality of enhancers are derived from two or more cancer-responsive genes that are either expressed at a higher level or are more active in cancer cells compared to non-cancer cells, wherein one of said plurality of enhancers comprises: (i) a transcription regulatory element with at least 90% sequence homology to an enhancer consensus sequence of two or more homologous cancer-responsive genes, and/or (ii) a sequence capable of binding a transcription associated protein as determined by chromatin immunoprecipitation (ChIP) or an in vitro transfection reporter assay.

In some aspects, provided herein is a recombinant polynucleotide comprising: (a) a core promoter comprising a transcription start site (TSS), wherein the core promoter is derived from one or more cancer-responsive genes that are either expressed at a higher level or are more active in cancer cells compared to non-cancer cells and operably linked to an open reading frame (ORF) and (b) a plurality of enhancers. In some embodiments, said plurality of enhancers are derived from one or more cancer-responsive genes that are either expressed at a higher level or are more active in cancer cells compared to non-cancer cells. In some embodiments, said plurality of enhancers are derived from two or more cancer-responsive genes that are either expressed at a higher level or are more active in cancer cells compared to non-cancer cells, wherein one of said plurality of enhancers comprises: (i) a transcription regulatory element with at least 90% sequence homology to an enhancer consensus sequence of two or more homologous cancer-responsive genes, and/or (ii) a sequence capable of binding a transcription associated protein as determined by chromatin immunoprecipitation (ChIP) or an in vitro transfection reporter assay.

In some aspects, provided herein, is a recombinant polynucleotide comprising: (a) a core promoter comprising a transcription start site (TSS), wherein the core promoter is derived from one or more cancer-responsive genes that are either expressed at a higher level or are more active in cancer cells compared to non-cancer cells and operably linked to an open reading frame (ORF), (b) a plurality of binding sites for one or more transcription factors (TFs), wherein said one or more TFs are expressed at higher levels or more active in cancer cells compared to non-cancer cells, and (c) a plurality of enhancers. In some embodiments, said plurality of enhancers are derived from one or more cancer-responsive genes that are either expressed at a higher level or are more active in cancer cells compared to non-cancer cells. In some embodiments, said plurality of enhancers are derived from two or more cancer-responsive genes that are either expressed at a higher level or are more active in cancer cells compared to non-cancer cells, wherein one of said plurality of enhancers comprises: (i) a transcription regulatory element with at least 90% sequence homology to an enhancer consensus sequence of two or more homologous cancer-responsive genes, and/or (ii) a sequence capable of binding a transcription associated protein as determined by chromatin immunoprecipitation (ChIP) or an in vitro transfection reporter assay.

In some aspects, provided herein is a recombinant polynucleotide comprising any of the sequences from Table 1A, Table 1B, or Table 1C. In some aspects, provided herein is a recombinant polynucleotide comprising a human alpha-fetoprotein (AFP) promoter sequence comprising a plurality of HNF-1A TF binding sites, wherein each HNF-1A binding site comprises the sequence 5′-GTTAATTATTAAC-3.′

In some aspects, provided herein is a vector comprising any of the recombinant polynucleotide described herein. In some aspects, provided herein is a pharmaceutical composition comprising any of the recombinant polynucleotide described herein or any the vector described herein and a pharmaceutically acceptable excipient, carrier, or diluents. In some aspects, provided herein is a lipid nanoparticle (LNP) comprising any of the recombinant polynucleotide described herein, any of the vector described herein, or any of the pharmaceutical composition described herein. In some aspects, provided herein is a cell comprising any the recombinant polynucleotide described herein, any of the vector described herein, any of the pharmaceutical composition described herein, or any of the LNP described herein.

In some aspects, provided herein is a method of selectively expressing a reporter protein in a cancer or tumor cell, comprising contacting said tumor cell with any of the recombinant polynucleotide described herein, any of the vector described herein, any of the pharmaceutical composition described herein, or any of the LNP described herein, wherein the recombinant polynucleotide further comprises an open reading frame (ORF) encoding said reporter protein, wherein said ORF is operatively linked to said synthetic promoter.

In some aspects, provided herein is a method comprising: (a) administering to a subject any of the pharmaceutical composition described herein; or a composition any of the recombinant polynucleotide described herein, any of the vector described herein, or any of the LNP described herein; wherein the recombinant polynucleotide further comprises an open reading frame (ORF) encoding a reporter protein, wherein said ORF is operatively linked to a synthetic promoter in said recombinant polynucleotide, and (b) detecting said reporter protein, wherein said pharmaceutical composition or said composition induces expression of said reporter protein preferentially in diseased cells in said subject compared to in non-disease cells, and wherein a relative ratio of said reporter protein expressed in said diseased cells over said non-diseased cells is greater than 1.0.

In some aspects, provided herein is a method for treating a subject having or suspected of having a disease, comprising administering to said subject any of the pharmaceutical composition described herein; or a composition any of the recombinant polynucleotide described herein, any of the vector described herein, or any of the LNP described herein; wherein the recombinant polynucleotide further comprises an open reading frame (ORF) encoding a therapeutic protein, wherein said ORF is operatively linked to a synthetic promoter in said recombinant polynucleotide, wherein said pharmaceutical composition or said composition induces expression of said therapeutic protein preferentially in diseased cells in said subject compared to in non-disease cells, and wherein a relative ratio of said therapeutic protein expressed in said diseased cells over said non-diseased cells is greater than 1.0.

In some aspects, provided herein is a method comprising: (a) administering to a subject any of the pharmaceutical composition described herein; or a composition any of the recombinant polynucleotide described herein, any of the vector described herein, or any of the LNP described herein; wherein the recombinant polynucleotide further comprises an open reading frame (ORF) encoding a reporter protein, wherein said ORF is operatively linked to a synthetic promoter in said recombinant polynucleotide, and (b) localizing a tumor or an absence thereof in a body of said subject via expression of said reporter protein using an imaging technique performed on said body of said subject.

In some aspects, provided herein is a method comprising: (a) introducing to a subject suspected of having a cancer via intravenous administration any of the pharmaceutical composition described herein; or a composition any of the recombinant polynucleotide described herein, any of the vector described herein, or any of the LNP described herein; wherein said recombinant polynucleotide further comprises an open reading frame (ORF) encoding a reporter protein, wherein said ORF is operatively linked to a synthetic promoter in said recombinant polynucleotide, and (b) detecting said reporter protein from said subject.

In some aspects, provided herein is a method comprising: (a) introducing to a subject suspected of having a cancer via intravenous administration a plurality of recombinant polynucleotides, wherein: said plurality of recombinant polynucleotides comprises a plurality of different promoters of genes overexpressed in a tumor cell versus a normal tissue or functional fragments thereof operably linked to genes encoding reporter proteins, wherein said plurality of different promoters of genes overexpressed in said tumor cell versus said normal tissue drive expression of said corresponding reporter proteins in a cell affected by said cancer, wherein said DNA molecules are selected from the group consisting of nanoplasmids and linear double-stranded DNA molecules; and (b) detecting said reporter proteins from said subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 shows a schematic of synthetic promoter architecture and design.

FIG. 2 describes coreCEACAM5 design.

FIG. 3 describes coreCEP55 design.

FIG. 4 describes coreFAM111B design.

FIG. 5 describes coreAGR2 design.

FIG. 6 shows the comparison of the reporter gene expression by endogenous promoter and synthetic promoter in H1299 cells.

FIG. 7 shows the reporter gene expression performance by synthetic promoters in human PDX models. Bar graphs from left to right: BIRC5, FOSL1-coreBIRC5, FOSL1-CEACAM5, FOSL1-FAM111B, FOSL1-KIF20A, FOSL1-AGR2, and FOSL1-TATA, respectively.

FIG. 8 shows signal-to-noise profiles of the reporter gene expression by synthetic promoters. Bar graphs from left to right: BIRC5, FOSL1-coreBIRC5, FOSL1-FAM111B, FOSL1-KIF20A, FOSL1-AGR2, FOSL1-CST1, and FOSL1-TATA, respectively.

FIG. 9 shows the reporter gene expression by synthetic promoters in H1299 cells.

FIG. 10 describes the workflow of synthetic promoter design and construction.

FIG. 11 describes the workflow of synthetic promoter design and construction with coreAGR2.

FIG. 12 describes the synthetic promoter architecture, design, discovery and validation pipeline.

FIG. 13 describes Transcription Factor Tile Design (top) and how to measure synthetic element expression (bottom). Each synthetic DNA sequence was designed as a series of repeated transcription factor (TF) binding sites derived from the consensus binding motif for the TF of interest (blue). To test the impact of the different relative positioning of these sites around the helical nature of the double stranded DNA (one helical turn is equivalent to ˜10.5 base pairs), the repeated binding sites are separated by a variable length of nucleic acid spacer sequences (yellow). Lastly, the synthetic DNA sequence contains a short filler sequence (grey) to maintain consistent total length of the candidate enhancer sequence block.

FIG. 14 shows Expression Score Distribution Across Lung Cancer Models. The expression score distribution varies across different lung cancer models. The PDX cell line LXFL430 had the widest distribution and outliers with the highest expression scores.

FIG. 15 shows the reporter gene expression by HOXC10 tiles. Using a luciferase reporter assay lead candidates representing the MNX1, HOXC10 and CREB3L1 transcription factors were tested across seven lung cancer cell line models (H1299, PDX430, PDX1121, PDX629, PDX529, PDX586, and PDX2184) and one lung normal cell line (IMR90). Higher expression compared to FOSL-coreBIRC5 lead synthetic promoter with up to 50-80 fold improvement was observed.

FIG. 16 shows the reporter gene expression by TCF7L1 TF tiles in PDX430 cell line.

FIG. 17 shows Wnt-driven cell lines identified by PCA (LK2 and NCI-H520) driving the expression by TCF7 and TCF7L1 promoters. In a transient transfection of two TCF7 variant promoters across five cell lines, H520 and LK-2 show the same high levels of activation as PDX430, which was predicted by the PCA analysis. As expected, H1299 and A549 cell lines do not show substantial expression by the TCF7 promoters, and are much better represented by the FOS-coreBIRC5 promoter.

FIG. 18 shows the expression of the reporter gene by TP53 elements. Addition of TP53 elements to TATA-TSS core results in significantly increased expression of the reporter gene in PDX586 as predicted by HTS-002.

FIG. 19 shows the expression of the reporter gene by TP53 variants in A549 cells.

FIG. 20 shows PCA analysis in H1944 and H2023 cells.

FIG. 21A shows a table comparing mutation status of P53, key gene set expression, and TP63 expression in different cancer cell lines.

FIGS. 21B and 21C show mutation profile in Clinical Proteomic Tumor Analysis Consortium (CPTAC) Lung adenocarcinoma (LUAD) and lung squamous cell carcinoma (LUSC), respectively.

FIG. 22 shows the reporter gene expression by p53 in A549, H1944, and H358 cell lines.

FIG. 23 shows a table comparing TP53 status and reporter gene expression in different cell lines.

FIG. 24 shows the reporter gene expression by TP53 and TCF7. Pathway specific TP53 and TCF7 response elements pair well and get higher signal using new non-coreBIRC5 cores. As observed with the FOS response element, TP53 and TCF7 response elements combined with coreCST1, coreAGR2, and coreFAM111B show up to a 10-fold signal increase compared to the same promoters constructed with coreBIRC5.

FIG. 25 shows the reporter gene expression by coreBIRC5 and coreAGR2 combined with different response elements in H1299, PDX430, and PDX586 cell lines.

FIG. 26 shows the reporter gene expression by coreBIRC5, coreAGR2, coreFAM111B combined with different response elements in different cell lines.

FIG. 27 shows fold change in expression of reporter genes from constructs comprising combination of FOSL and CREB3L1.

FIG. 28 shows fold change in expression of reporter genes from constructs comprising combination of TCF7 and TP53.

FIG. 29 shows validation of top ranked TF tiles with the coreBIRC5 promoter. Using a luciferase reporter assay various TF tiles that were highly ranked in the MPRA screens for H1299 and LXFL430 were tested. Many of the TF tiles showed stronger expression than the base expression of the coreBIRC5 and the FOSL-coreBIRC5. The TCF7L1 TF tiles showed specific expression in the LXFL430 cell line.

FIGS. 30A and 30B show expression of synthetic promoter FOS-coreBIRC5 in PDX cell lines and normal lung cell lines. Compared to endogenous promoters, including the Survivin (BIRC5) promoter and other first-generation endogenous promoters used in multiplexes, the synthetic promoter FOS-coreBIRC5 outperformed in terms of strength and sensitivity in 8 PDX cell lines that represent different patients' genomic profiles (FIG. 30A). FIG. 30B shows that the synthetic promoter also demonstrates lack of expression in normal human fibroblast cell line (IMR-90), small airway epithelial cells (SAEC) and normal human bronchial epithelial cells (NHBE).

FIG. 31 shows the top 30 contributing features that make up a factor of MOFA analysis.

FIG. 32 shows comparison of reporter gene expression by FOSL2 in Normal Adjacent Tissues (NAT) and tumor.

FIG. 33 shows the binding of FOSL2 and C-Jun TFs to the FOS element in the FOS-coreBIRC5 promoter. Chromatin immunoprecipitation (ChIP) was performed on two different cell lines transfected with the FOS-coreBIRC5 promoter construct. Pulldowns for FOSL2 and c-Jun showed significant enrichment of the coreBIRC5 element compared to nonspecific pulldown, by 14× for FOSL2 in H1299 and 5× for FOSL2 in A549. With the comparison to the control construct of solely coreBIRC5, this makes it clear that the FOS response element is responsible for the association of FOSL2 and C-Jun with the synthetic promoter.

FIG. 34 shows demonstration of high sensitivity and specificity in primary-derived and commercial cell lines by chimeric promoters using core-BIRC5. Response elements for different TFs (FOSL2, TWIST1, ETV4) in combination with the coreBIRC5 promoter showed variable sensitivity across different PDX cell lines, H1299 NSCLC cell line, and a lack of expression in IMR-90 (normal human fibroblast) cell line.

FIG. 35 shows the activity of TCF7 & TCFL1 variants in different cell lines. TCF7 & TCFL1 variants were only active in PDX LXFL430 among cell lines tested. Two variants of the TCF7-response element promoter, as compared to the minimal coreBIRC5 and positive control FOS-coreBIRC5 promoter, demonstrated extremely high levels of expression in the large cell lung cancer PDX430.

FIG. 36 shows that alternative core promoters to coreBIRC5 demonstrate high utility in synthetic promoter constructs. The full-length endogenous promoters, core promoters, and FOS-core promoters using BIRC5, FAM111B, AGR2 and CST1 were tested in two lung cancer cell lines—H1299 and PDX629. The use of the new cores with FOS demonstrated up to 20-fold improvement in signal compared to the original FOS-coreBIRC5 promoter described previously. On the bottom, experiments using three primary normal lung cell lines (small airway epithelial cells from two donors and normal human lung fibroblasts) demonstrated the FOS-coreAGR2 and FOS-coreCST1 constructs still maintain high specificity for cancer, while FOS-coreFAM111B appears to have significant noise in lung fibroblasts.

FIG. 37 shows reporter gene expression derived by different synthetic promoters in cancer epithelial cells, cancer associated fibroblast cells, and normal adjacent tissue (NAT) cells from patient derived cell lines (LU057: 63/F/White, Stage IIIB Adeno-squamous pT4, N2). *: not tested. dotted line: CAG, constitutive promoter.

FIGS. 38A and 38B show AFP-3, an engineered variant of the human alpha-fetoprotein (AFP) promoter that can drive strong and highly specific expression in HCC. In FIG. 38A, the primary changes to the AFP promoter sequence are shown, changing the HNF-1A sites to the consensus sequence for the transcription factor binding site. FIG. 38A discloses SEQ ID NOs: 553-554, respectively, in order of appearance. FIG. 38B shows that engineered AFP-3 drives up to 200-fold higher expression in liver cancer cell lines than the wildtype AFP promoter, while still maintaining high specificity against lung normal (IMR-90, MRC-9), lung cancer (H1299) and melanoma (MeWo) cell lines, as compared to the Survivin (BIRC5) promoter which shows some cancer-activated activity in both liver and non-liver cancer cell lines.

FIG. 39 shows signal-to-noise ratio of SEAP in Hep3B orthotopic tumor model. Secreted alkaline phosphatase (SEAP) was measured from the serum of tumor-bearing and normal animals dosed with the BIRC5-SEAP construct versus the AFP-3-SEAP construct. At the day 0 bleed (pre-dosing), background levels of SEAP in all mice were below the lower limit of quantification (LLOQ) of the assay (0.4 pg/12.5 uL), as expected. At 3 days post-dose, the BIRC5-SEAP construct dosed animals showed a 7-fold increase of SEAP reporter in the serum over the LLOQ, with no background expression at all in non-tumored animals. The AFP-3 construct promoted expression in tumored animals approximately 97-fold higher than non-tumored animals.

FIGS. 40A, 40B, and 40C show immunohistochemistry (IHC) results for AFP-3-sr39tk, using HA epitope. FIGS. 40A and 40B show representative serial sections from the tumor-bearing left lobe of a mouse in Group 6 (AFP-3-sr39tk) dosed at 2.8mpk of EM-40 stained by H&E and by HA antibody for the reporter expression. The tumor boundary has been outlined in the H&E slide. Reporter expression is confined to the tumor cells only. In FIG. 40C, the same mouse's right liver lobe, devoid of tumor is shown to have no positive cells.

FIGS. 41A, 41B, 41C, 41D, 41E, and 41F show IHC results for positive control CAG-sr39tk. Serial sections of the tumor-containing left lobe from a mouse in Group 10 show positive staining in the tumor (FIGS. 41A and 41B; stained dark purple by H&E). Left and right lobe sections from the same mouse show occasional disperse signal from individual cells (FIGS. 41C and 41D). Serial sections stained by H&E and by IHC for the −HA tag for a second mouse's tumor also show many positive-stained cells throughout the tumor tissue, as outlined in the H&E figure (FIGS. 41E and 41F).

FIG. 42 shows images of animal bioluminescence.

FIGS. 43A, 43B, 43C, and 43D show muti-omics data on benign cell lines.

FIG. 44 shows that there is no reporter expression by synthetic promoter constructs in granulomatous lesions caused by Mycobacterium tuberculosis (M. tb) infection in CBA/J mice despite high disease burden.

FIG. 45 shows the reporter gene expression performance by different synthetic promoters in various cancer and non-cancer cell lines. Combining the FOS element with new core promoters resulted in significant increases in expression across NSCLC cell lines & PDX CL models. Bar graphs from left to right: HIGH-coreBIRC5, FOS-coreBIRC5, FOS-CEACAM5, FOS-FAM111B, FOS-KIF20A, FOS-AGR2, FOS-CST, and FOS-TATA, respectively.

FIG. 46 shows the reporter gene expression performance by different synthetic promoters in various cancer and non-cancer cell lines. Some FOS-newCores combinations had elevated noise in Normal Lung Fibroblasts. Bar graphs from left to right: FOS-BIRC5, FOS-CEACAM5, FOS-FAM111B, FOS-KIF20A, FOS-AGR2, FOS-CST1, and FOS-TATA, respectively.

FIG. 47 shows an exemplary workflow of diagnostic medical sonography (DMS) study.

FIG. 48 shows a schematic of adding activating elements to the new core promoters.

FIG. 49 shows the reporter gene expression performance by different synthetic promoters in H1299 and PDX430 cell lines. HIGH element was observed to be functional in vitro when combined with alternate core promoters. Bar graphs from left to right: BIRC5, CEACAM5, FAM111B, KIF20A, AGR2, and FOS-TATA, respectively.

FIG. 50 shows the reporter gene expression performance by different synthetic promoters in normal small airway epithelial cells and normal lung fibroblasts. In vitro specificity models were predictive of lung noise with HIGH-CEACAM5, HIGH-FAM111B and HIGH-KIF20A. Bar graphs from left to right: HIGH-BIRC5, HIGH-CEACAM5, HIGH-FAM111B, HIGH-KIF20A, HIGH-AGR2, FOS-AGR2, and FOS-TATA, respectively.

FIG. 51 shows the reporter gene expression performance by different synthetic promoters in various PDX cell lines. Synthetic promoters described herein outperform endogenous promoter in PDX cell lines. Bar graphs from left to right: Survivin (endogenous BIRC5 promoter), FOS-coreBIRC5, HIGH-coreBIRC5, FOS-coreAGR2, FOS-coreCST1, HIGH-FAM111B, FOS-TATA-TSS, and EF1A (positive control), respectively.

FIG. 52 shows the reporter gene expression performance by different synthetic promoters in various primary cell lines derived from PDX or primary tissue. Bar graphs from left to right: Survivin (endogenous BIRC5 promoter), FOS-coreBIRC5, HIGH-coreBIRC5, FOS-coreAGR2, FOS-coreCST1, HIGH-FAM111B, FOS-TATA-TSS, and CAG (positive control), respectively.

FIG. 53 shows the reporter gene expression performance by different synthetic promoters in primary lung normal cells (Lonza). Bar graphs from left to right: Survivin (endogenous BIRC5 promoter), FOS-coreBIRC5, HIGH-coreBIRC5, FOS-coreAGR2, FOS-coreCST1, HIGH-FAM111B, FOS-TATA-TSS, and EF1A (positive control), respectively.

FIG. 54 shows the reporter gene expression performance by different synthetic promoters in different primary lung normal cells derived from the same patient.

FIG. 55 shows the comparison of the reporter gene expression performance by synthetic promoters in EMT state cells and wild type A549 cells.

FIG. 56 shows a table of top 10 enhancer candidates.

FIG. 57 shows the reporter gene expression performance by synthetic promoters comprising enhancer elements in various cancer and non-cancer cells. Constructs were tested in vitro across panel of 5 LUAD cell lines, 3 HCC cell lines, and IMR90 lung normal cells for expression profiles of enhancer elements paired with each core promoter (including 7× CRL PDX cell lines and 2× Lonza normal cells).

FIG. 58 shows comparison of the reporter gene expression performance by different synthetic promoters comprising enhancer elements in various cancer cell lines.

FIG. 59 shows the reporter gene expression performance by different synthetic promoters in various cell lines. Bar graphs from left to right: BIRC5, Canscript, FOSL1, GATA1, MYC_MAX, SOX9, AFP, AFP3, Enhancer+AFP3, and NT EF1a, respectively.

FIG. 60 shows a two-step promoter amplification utilizing the yeast GAL4-VP system.

FIG. 61 shows comparison of the reporter gene expression performance by different synthetic promoters and the yeast GAL4-VP system in H1299, LXFA 629, and LXFA 737 cell lines. TSTA: two-step transcriptional activation. Bar graphs from left to right: EF1A, CMV, BIRC5, FOSL1, AFP3, TSTA PR-GAL4 only, BIRC5, FOSL1, AFP3, respectively.

FIG. 62 shows comparison of the reporter gene expression performance by different synthetic promoters and the yeast GAL4-VP system in SNU-475, PLC/PRF/5, and C3A cell lines. TSTA: two-step transcriptional activation. Bar graphs from left to right: EF1A, CMV, BIRC5, FOSL1, AFP3, TSTA PR-GAL4 only, BIRC5, FOSL1, AFP3, respectively.

FIG. 63 shows exemplary core promoters with annotations. FIG. 63 discloses SEQ ID NO: 555.

FIG. 64A shows a diagram of an annotated core FAM111B promoter with predicted TF binding sites.

FIG. 64B shows activating and repressing elements within coreFAM111B identified from core promoter element deletion studies.

FIG. 65 shows top 10 ranked response elements from H1299 (Large Cell Carcinoma), LXFA586 (Adenocarcinoma), and LXFL430 (Large Cell Carcinoma). Control response elements containing FOS/CREB (H1299), TP53/TP73 (LXFA586), or TCF (LXFL430) drive strong expression of reporter gene in H1299, LXFA586, and LXFL430 cell lines respectively, and there are several additional hits.

FIGS. 66A, 66B, 66c, and 66D show in vitro low throughput validation of response elements from FIG. 112 using Firefly luciferase (FLuc) assay.

FIGS. 67-68 show a DNA binding consensus sequence of Forkhead Box Protein O1 (FOXO1; FIG. 67, left), ELK3 (FIG. 67, middle), FOXO::ELK (FIG. 67, right), XBP1 (FIG. 68, top left), NFE2L2 (FIG. 68, top right), and MTF1 (FIG. 68, bottom).

FIG. 69 shows validation of response elements with FOS and CREB using Firefly luciferase (FLuc) assay.

FIG. 70 shows Firefly luciferase (FLuc) assay results of combination of TCF and FOS elements.

FIG. 71 shows Firefly luciferase (FLuc) assay results of different elements in patient-derived cancer cells (cancer epithelia and cancer fibroblasts) and normal adjacent tissues. Bar graphs from left to right: Cancer Epithelia, Cancer Fibroblasts, and Normal Adjacent Tissues, respectively.

FIG. 72 shows Synthetic Response Sensors (SRS) that drive cancer specific expression where the SRS comprises a series of Synthetic Response Elements (SREs), or enhancers, and a cancer activated core promoter. TF: Transcription Factor.

FIG. 73 shows a graph of gene expression activated by SRS-G comprising the core promoter specific for lung cancer and a single SRE. A luciferase reporter expression system was used to evaluate the strength of activation in cell lines that represent the three main Non-Small Cell Lung Cancer (NSCLC) subtypes. The expression values are shown as the fold change over a strong constitutive promoter. SRS-G was able to achieve expression that is 10-20% on the expression of the constitutive promoter.

FIGS. 74A, 74C, 74E, 74G, 74I, and 74K show graphs of gene expression activated by different SRSs (SRS-A, SRS-B, SRS-C, SRS-D, SRS-E, and SRS-F) designed to drive gene expression in lung cancers. A luciferase reporter expression system was used to evaluate the strength of activation in cell lines that represent the three main NSCLC subtypes. The expression values are shown as the fold change over a strong constitutive promoter. SRS-A was able to achieve expression that is 5-50% on the expression of the constitutive promoter (FIG. 74A). SRS-B was able to achieve expression that is 20-50% on the expression of the constitutive promoter (FIG. 74C). SRS-C was able to achieve expression similar to or 3-fold above the constitutive promoter (FIG. 74E). SRS-D was able to achieve expression similar to or 2-10-fold above the constitutive promoter (FIG. 74G). SRS-E was able to achieve expression similar to or 2-8-fold above the constitutive promoter (FIG. 74I). SRS-F was able to achieve expression similar to or 3-5-fold above the constitutive promoter. (FIG. 74K).

FIGS. 74B, 74D, 74F, 74H, 74J, and 74L show graphs of gene expression activated by an SRS designed to drive gene expression in lung cancers (SRS-A, SRS-B, SRS-C, SRS-D, SRS-E, and SRS-F). A luciferase reporter expression system was used to evaluate the strength of activation in cell lines that represent the NSCLC subtypes as well as normal primary lung cells. Expression values are shown as the fold change over a strong constitutive promoter on the left. Same data plotted as an ROC curve is presented on the right.

FIG. 75 shows graphs of expression pattern of a reporter gene activated by a constitutive or non-cancer specific promoter, Cytomegalovirus (CMV). A luciferase reporter expression system was used to evaluate the strength of activation in cell lines that represent the NSCLC subtypes as well as normal primary lung cells. Expression values are shown as the fold change over a strong constitutive promoter on the left. Same data plotted as an ROC curve is presented on the right.

FIG. 76 shows graphs of gene expression activated by SRSs, demonstrating that SRSs can be active in both lung and liver cancer models, or selectively active in a target model. H358 lung cancer cells, HepG2 liver cancer cells, and Hep3B liver cancer cells were seeded in 96-well plates at a density of 10,000 cells per well, with each plasmid containing luciferase reporter expression system tested in triplicate. Transfection was performed using Lipofectamine 3000, following the manufacturer's protocol. After 24 hours of incubation, expression levels were measured using the Promega Luciferase Assay System (E1501). The expression values are shown as the fold change over a strong constitutive promoter, where greater than 10% expression is set as a threshold for positive signal. The results demonstrate that SRS-G and SRS-B are active in both lung and liver cancer cell lines, whereas SRS-H, a liver-specific promoter, is active only in liver cancer cell lines.

FIG. 77 shows a graph of gene expression activated by SRSs in different tissues, illustrating the in vivo performance of several SRSs when administered via intravenous (i.v.) bolus to tumor-bearing mice. Quantification of firefly bioluminescence of tissues ex vivo was taken 24 hours after compound dosing normalized to the average bioluminescence imaging (BLI) of PBS dosed animals (n=3, dotted line set at 1). Plotted by dosing group with each tissue in column. Each point represents a tissue from a unique animal. Circles: CAG constitutive promoter; squares: SRS-F; triangles: SRS-I; diamonds: SRS-E; stars: SRS-J. Error bars represent standard error of the mean (SEM). Tables on the bottom show calculated signal to noise ratios (SNR) for a given promoter over potential background noise tissues (liver, spleen) demonstrating improved SNR and selectivity for synthetic promoters relative to constitutively active CAG promoter.

FIG. 78 shows a graph of reporter gene expression under different SRSs compared to a constitutive promoter. A FLUC reporter readout was used to assess specificity of SRSs comprising combinations of different promoters and SREs in lung cancer (H1299) and two different normal lung cell lines (Lung Normal 1 and Lung Normal 2). Reporter expression under SRS-K (using the non-specific promoter TATA-TSS) was high in both lung cancer and normal cell lines. Reporter expression under SRS-L and SRS-M was lower in all cell lines compared to that under SRS-K, especially in normal cell lines. Specifically, reporter gene expression under SRS-L was reduced 2× in cancer cell line and 10-20× in normal cell lines compared to reporter gene expression under SRS-K, which comprises non-specific promoter TATA-TSS, indicating that core promoters provide selectivity and specificity for cancer cells compared to normal cells.

### DETAILED DESCRIPTION

The compositions and methods described herein contemplates a general strategy of identifying important elements of cancer-specific (or cancer-activated) promoters and designing and/or engineering cancer-specific promoters using elements of cancer-specific promoters identified. Cancer-specific promoters or cancer-activated promoters described herein can comprise promoters of genes that are preferentially expressed in cancer cells compared to non-cancer cells or expressed in higher level in cancer cells compared to non-cancer cells. Methods described herein can comprise identifying endogenous cancer-activated promoters by evaluating candidate promoter and/or enhancer sequences using bioinformatic analysis and designing/engineering a minimal cancer-activated promoter sequence (core promoter). For example, a candidate sequence (e.g., low-throughput or high-throughput screening) can be examined using a genome browser. The assessment range (e.g., sequence boundary) can be set based on the predicted transcriptional start site (TSS) of an endogenous promoter. For example, the assessment range can be from about −1000 bp to about +1000 bp relative to the predicted TSS. The assessment range can be adjusted based on chromatin immunoprecipitation (ChIP) data including, but not limited to, ChIP peaks of general transcription factors (TFs), indicators of active promoter regions, and TFs that may indicate cancer specificity by presence in cancer cells and absence in non-cancer cells; and abundance of predicted TF binding sequence (TFBS); and regions of high species conservation. In some embodiments, indicators of active promoter regions can include, but not limited to, RNA Polymerase II, DNAse I, H3K4me1, and H3K4me3. In some embodiments, TFBS abundance can be predicted using methods including, but not limited, to JASPAR or HOMER motif analysis. Methods described herein can also comprise testing highlight regulated TFs using Massively Parallel Reporter Assay (MPRA) to identify optimal sequences, optimal spacing between each sequence, and/or optimal combinations of different enhancer sequences to design synthetic tiled enhancers. Methods described herein can comprise a rationally designed (e.g., low-throughput) screening or a high-throughput screening to identify enhancer elements to increase transcription signal. In some embodiments, a synthetic tiled enhancer can comprise one or more copies of TFBS, or other highly conserved regulatory element repeats with spacing between repeats. One or more synthetic elements described herein can be placed upstream of core promoters. Synthetic elements described herein can also function as a promoter without a promoter or a core promoter.

A cancer-specific promoter described herein can comprise a recombinant polynucleotide comprising a core promoter sequence comprising a transcription start site (TSS). In some embodiments, a core promoter can be derived from a cancer-responsive gene and can be operably linked to an open reading frame (ORF). In some embodiments, a cancer-responsive gene can comprise a human cancer-responsive gene. In some embodiments, a core promoter can comprise a plurality of binding sites for a plurality of transcription factors (TFs) that are expressed in higher levels in cancer cells compared to non-cancer cells. In some embodiments, a core promoter can comprise a plurality of binding sites for a plurality of transcription factors (TFs) that are more active in cancer cells compared to non-cancer cells. In some embodiments, a core promoter can comprise a plurality of enhancers derived from two or more human cancer-response genes. In one embodiment, each of the plurality of enhancers can comprise a transcription regulatory element with at least 80% sequence homology to the enhancer consensus sequence of the two or more human cancer-response genes. In another embodiment, each of the plurality of enhancers can comprise a sequence capable of binding a transcription associated protein as assessed by ChIP.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below.

### Definitions

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. The terms “and/or,” “a combination thereof,” and “any combination thereof”′ and their grammatical equivalents as used herein, can be used interchangeably. These terms can convey that any combination is specifically contemplated. Solely for illustrative purposes, the following phrases “A, B, and/or C,” “A, B, C, or a combination thereof,” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value, which may depend in part on how the value is measured or determined, e.g., the limitations of the measurement system. For example, “about” can mean within 1 or more than 1 standard deviation. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the present disclosure, subject to any specifically excluded limit in the stated range. Where the stated range includes one or both of the limits, ranges excluding either or both of those included limits are also included in the present disclosure, unless the context clearly dictates otherwise.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “embodiments,” “certain embodiments,” “preferred embodiments,” “specific embodiments,” “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Certain specific details of this description are set forth in order to provide a thorough understanding of various embodiments. However, one skilled in the art will understand that the present disclosure may be practiced without these details. In other instances, well-known techniques or methods have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

The terms “nucleic acid sequence,” “polynucleic acid sequence,” and/or “nucleotide sequence” are used herein interchangeably and have the identical meaning herein and refer to DNA or RNA. In some embodiments, a nucleic acid sequence is a polymer comprising or consisting of nucleotide monomers, which are covalently linked to each other by phosphodiester-bonds of a sugar/phosphate-backbone. The terms “nucleic acid sequence,” “polynucleic acid sequence,” and “nucleotide sequence” may encompass unmodified nucleic acid sequences, i.e., comprise unmodified nucleotides, or natural nucleotides. In some embodiments, “natural nucleotide,” “unmodified nucleotide,” and/or “canonical nucleotide” are used herein interchangeably and have the identical meaning herein and refer to the naturally occurring nucleotide bases adenine (A), guanine (G), cytosine (C), uracil (U), and/or thymine (T). The terms “nucleic acid sequence,” “polynucleic acid sequence,” and “nucleotide sequence” may also encompass modified nucleic acid sequences, such as base-modified, sugar-modified or backbone-modified etc., DNA or RNA. The term “nucleic acid sequence” generally is understood to include, as applicable to the embodiment being described, single-stranded (such as sense or antisense) and double-stranded polynucleotides. The term “nucleic acid” generally is understood to include, as applicable to the embodiment being described, polymers containing a non-natural linkage or a non-natural nucleotide.

In some embodiments, a nucleic sequence acid as described herein comprises one or more non-natural linkages or one or more non-natural nucleotides. Non-natural nucleotides can include, but are not limited to, 2′-fluoro, 2′-O-methyl, 2′-O-methyl, 2′-O-methoxy-ethyl, 2′-O-methoxy-ethoxy, 5′-methyl, SNA, hGNA, hhGNA, mGNA, TNA, h′GNA, locked nucleic acids (LNAs), GNA-isoC, GNA-isoG, 5′-mUNA, 4′-mUNA, 3′-mUNA, 2′-mUNA, or an abasic nucleotide (e.g. DNA or RNA). Non-natural linkages can include, but are not limited to, phosphorothioate and methylphosphonate. In some embodiments, an oligonucleotide as described herein comprises a modified uracil. Example nucleobases and nucleosides having a modified uracil include pseudouridine (Ψ), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridine or 5-bromo-uridine), 3-methyl-uridine (m3U), 5-methoxy-uridine (mo5U), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (tm5U), 1-taurinomethyl-pseudouridine, 5-taurinomethyl-2-thio-uridine (m5s2U), 1-taurinomethyl-4-thio-pseudouridine, 5-methyl-uridine (m5U, i.e., having the nucleobase deoxythymine), 1-methylpseudouridine (m1_{ψ}), 5-methyl-2-thio-uridine (m5s2U), 1-methyl-4-thio-pseudouridine (m1s4_{ψ}), 4-thio-1-methyl-pseudouridine, 3-methyl-pseudouridine (m3_{ψ}), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine (aka 1-methylpseudouridine (m1_{ψ})), 3-(3-amino-3-carboxypropyl) uridine (acp3U), 1-methyl-3-(3-amino-3-carboxypropyl) pseudouridine (acp3 ψ), 5-(isopentenylaminomethyl) uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (ψm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino) uridine. In some embodiments, an oligonucleotide as described herein comprises a modified cytosine. Example nucleobases and nucleosides having a modified cytosine include 5-aza-cytidine, 6-aza-cytidine, pseudoisocytidine, 3-methyl-cytidine (m3C), N4-acetyl-cytidine (ac4C), 5-formyl-cytidine (f5C), N4-methyl-cytidine (m4C), 5-methyl-cytidine (m5C), 5-halo-cytidine (e.g., 5-iodo-cytidine), 5-hydroxymethyl-cytidine (hm5C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k2C), α-thio-cytidine, 2′-O-methyl-cytidine (Cm), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m4 2 Cm), 1-thio-cytidine, 2′-F-aracytidine, 2′-F-cytidine, and 2′-OH-aracytidine

The term “subject” can generally include human or non-human animals. Thus, the methods and compositions described herein are applicable to both human and veterinary disease and animal models. Preferred subjects are “patients,” i.e., living humans that are receiving medical care for a disease or condition (e.g., cancer). This includes persons with no defined illness who are being investigated for signs of pathology. Also included are persons suspected of possessing or being at-risk for a defined illness. In some embodiments, the subject has at least one risk factor for cancer.

A “vector” as used herein generally refers to a nucleic acid sequence capable of transferring other operably-linked heterologous or recombinant nucleic acid sequences to target cells. In some examples, a vector is a minicircle, plasmid, nanoplasmid, yeast artificial chromosome (YAC), bacterial artificial chromosome (BAC), cosmid, phagemid, bacteriophage genome, or baculovirus genome. Suitable vectors also include vectors derived from bacteriophages or plant, invertebrate, or animal (including human) viruses such as CELiD vectors, doggybone DNA (dbDNA) vectors, closed-end linear duplex DNA vectors (e.g., wherein each end is covalently closed by chemical modification), adeno-associated viral vectors (e.g., AAV1, AAV2, AAV4, AAV5, AAV6, AAV7, AAV8, AAV9, or pseudotyped combinations thereof such as AAV2/5, AAV2/2, AAV-DJ, or AAV-DJ8), retroviral vectors (e.g. MLV or self-inactivating or SIN versions thereof, or pseudotyped versions thereof), herpesviral (e.g. HSV- or EBV-based), lentiviral vectors (e.g., HIV-, FIV-, or EIAV-based, or pseudotyped versions thereof), or adenoviral vectors (e.g., Ad5-based, including replication-deficient, replication-competent, or helper-dependent versions thereof). In some embodiments, a vector is a replication competent viral-derived vector. In some embodiments, a vector is a replication-incompetent viral-derived vector. In some cases, the vector may comprise an episomal maintenance element to facilitate replication in one or more target cell type, such as a Scaffold/Matrix Attachment Region (S/MAR). S/MAR elements are particularly useful to facilitate replication in the context of “naked” nucleic acid vectors such as minicircles. Exemplary suitable S/MAR elements include, but are not limited to, EμMAR from the immunoglobulin heavy chain locus, the apoB MAR from the human apolipoprotein B locus, the Ch-LysMAR from the chicken lysozyme locus, and the huIFNβ MAR from the human IFNβ-locus. A vector may comprise a coding sequence capable of being expressed in a target cell. Accordingly, as used herein, the terms “vector construct,” “expression vector,” and “gene transfer vector,” may refer to any nucleic acid construct capable of directing the expression of a gene of interest and which is useful in transferring the gene of interest into target cells. Vectors as described herein may additionally comprise one or more cis-acting elements to stabilize or improve expression of mRNAs therefrom. Such cis-acting elements include, but are not limited to, any of the elements described e.g., in Johansen et al. The Journal of Gene Medicine. (5) 12:1080-1089 (doi: 10.1002/jgm.444) or Vlasova-St. Louis and Sagarsky. Mammalian Cis-Acting RNA Sequence Elements (doi: 10.5772/intechopen.72124).

The term “promoter” generally can refer to a DNA sequence that directs the transcription of a polynucleotide. Typically, a promoter can be located in the 5′ region of a polynucleotide to be transcribed, proximal to the transcriptional start site of such polynucleotide. More typically, promoters can be defined as the region upstream of the first exon; more typically, as a region upstream of the first of multiple transcription start sites. Frequently promoters are capable of directing transcription of genes located on each of the complementary DNA strands that are 3′ to the promoter. Stated differently, many promoters can exhibit bidirectionality and can direct transcription of a downstream gene when present in either orientation (i.e., 5′ to 3′ or 3′ to 5′ relative to the coding region of the gene). Additionally, the promoter may also include at least one control element such as an upstream element. Such elements include upstream activator regions (UARs) and optionally, other DNA sequences that affect transcription of a polynucleotide such as a synthetic upstream element. Some promoters may be assembled from fragments of endogenous promoters (e.g., derived from the human genome).

The term “coding sequence,” and “encodes” when used in reference to a polypeptide herein generally refer to a nucleic acid molecule that is transcribed (in the case of DNA) and translated (in the case of mRNA) into a polypeptide, for example, when the nucleic acid is present in a living cell (in vivo) and placed under the control of appropriate regulatory sequences (or “control elements”). The boundaries of the coding sequence are typically determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral, eukaryotic, or prokaryotic DNA, and synthetic DNA sequences. A transcription termination sequence may be located 3′ to the coding sequence, and a promoter may be located 5′ to the coding sequence; along with additional control sequences if desired, such as enhancers, introns, poly adenylation site, etc. A DNA sequence encoding a polypeptide may be optimized for expression in a selected cell by using the codons preferred by the selected cell to represent the DNA copy of the desired polypeptide coding sequence.

The term “operably linked” as used herein generally can refer to an arrangement of elements wherein the components so described are configured so as to perform their usual function. Thus, a given promoter that is operably linked to a coding sequence (e.g., a reporter expression cassette) is capable of effecting the expression of the coding sequence when the proper enzymes are present. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

The term “sequence identity” or “percent identity” in the context of two or more nucleic acids or polypeptide sequences, generally refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation (E) of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation (E) of 1000000, and the PAM30 scoring matrix setting gap costs at 9 to open gaps and 1 to extend gaps for sequences of less than 30 residues (these are the default parameters for BLASTP in the BLAST suite available at https://blast.ncbi.nlm.nih.gov); CLUSTALW with parameters of; the Smith-Waterman homology search algorithm with parameters of a match of 2, a mismatch of −1, and a gap of −1; MUSCLE with default parameters; MAFFT with parameters retree of 2 and maxiterations of 1000; Novafold with default parameters; HMMER hmmalign with default parameters.

The term “lipid particle” generally includes a lipid formulation that can be used to deliver an active agent or therapeutic agent, such as a nucleic acid to a target site of interest (e.g., cell, tissue, organ, and the like). In preferred embodiments, the lipid particle of the invention is a nucleic acid-lipid particle (e.g. a particle that has only nucleic acids and lipids), which is typically formed from a cationic lipid, a non-cationic lipid, and optionally a conjugated lipid that prevents aggregation of the particle. In other preferred embodiments, the active agent or therapeutic agent, such as a nucleic acid, may be encapsulated in the lipid portion of the particle, thereby protecting it from enzymatic degradation. In some cases, a “lipid particle” is a lipid nanoparticle (LNP). The lipid particles can be prepared by any suitable method, including but not limited to microfluidic assembly or extrusion. In some embodiments, for a lipid particle (e.g. LNP composition), a particle has a particular composition. In some embodiments, for a lipid particle (e.g. LNP composition), each particle has a particular composition. In some embodiments, for a lipid particle (e.g. LNP composition), at least 1%, 5%, 10%, 15%, 20%, 25%, 30%, 35%, 40%, 45%, 50%, 55%, 60%, 65%, 70%, 75%, 80%, 85%, 90%, 95%, 98%, 99%, 99.5%, 99.9% of the particles have a particular composition.

When nucleic acid sequences are referred to herein, the current disclosure is generally understood to include nucleic acid sequences with at least about 80-100% identity to the sequences described herein, or to reverse complements of the sequences described herein.

In some embodiments, the disclosure provides for a nucleic acid comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% sequence identity to any of the sequences listed in Table 1A, or to reverse complements of any of the sequences listed in Table 1A. In some embodiments, the disclosure provides for a nucleic acid comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% sequence identity to any of SEQ ID NOs: 1-343, or to reverse complements of any of SEQ ID NOs: 1-343. In some embodiments, the disclosure provides for a promoter comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% sequence identity to any of SEQ ID NOs: 1-343, or to reverse complements of any of SEQ ID NOs: 1-343. In some embodiments, the nucleic acid can be a double-stranded nucleic acid.

In some embodiments, the disclosure provides for a nucleic acid comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% sequence identity to any of the sequences listed in Table 1B, or to reverse complements of any of the sequences listed in Table 1B. In some embodiments, the disclosure provides for a nucleic acid comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% sequence identity to any of SEQ ID NOs: 377-397, or to reverse complements of any of SEQ ID NOs: 377-397. In some embodiments, the disclosure provides for a promoter comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% sequence identity to any of SEQ ID NOs: 377-397, or to reverse complements of any of SEQ ID NOs: 377-397. In some embodiments, the disclosure provides for an enhancer comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% sequence identity to any of SEQ ID NOs: 377-397, or to reverse complements of any of SEQ ID NOs: 377-397. In some embodiments, the nucleic acid can be a double-stranded nucleic acid.

In some embodiments, the disclosure provides for a nucleic acid comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% sequence identity to any of the sequences listed in Table 1C, or to reverse complements of any of the sequences listed in Table 1C. In some embodiments, the disclosure provides for a nucleic acid comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% sequence identity to any of SEQ ID NOs: 398-488, or to reverse complements to any of SEQ ID NOs: 398-488. In some embodiments, the disclosure provides for a promoter having a sequence having at least 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% sequence identity to any of the sequences listed in Table 1C, or to reverse complements of any of the sequences listed in Table 1C. In some embodiments, the disclosure provides for a promoter comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% sequence identity to any of SEQ ID NOs: 398-486 and SEQ ID NOs: 556-557, or to reverse complements to any of SEQ ID NOs: 398-486 and SEQ ID NOs: 556-557. In some embodiments, the nucleic acid can be a double-stranded nucleic acid.

In some embodiments, the disclosure provides for a nucleic acid comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% sequence identity to any one of the of the sequences listed in Table 1J, or to reverse complements of any one of the sequences listed in Table 1J. In some embodiments, the disclosure provides for a nucleic acid comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% sequence identity to any SEQ ID NOs: 558-587, or to any reverse complements of any SEQ ID NOs: 558-587. In some embodiments, the disclosure provides for a core promoter comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% sequence identity to any one of the of the sequences listed in Table 1J, or to reverse complements of any one of the sequences listed in Table 1J. In some embodiments, the disclosure provides for the core promoter comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% sequence identity to any SEQ ID NOs: 558-587, or to any reverse complements of any SEQ ID NOs: 558-587. In some embodiments, the nucleic acid can be a double-stranded nucleic acid.

In some embodiments, the disclosure provides for a nucleic acid comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% sequence identity to SEQ ID NO: 556, listed in Table 1C, or to a reverse complement thereof. In some embodiments, the nucleic acid can be a double-stranded nucleic acid.

In some embodiments, the disclosure provides for a nucleic acid comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% sequence identity to SEQ ID NO: 557, listed in Table 1C, or to a reverse complement thereof. In some embodiments, the nucleic acid can be a double-stranded nucleic acid.

In some embodiments, any of the nucleic acids disclosed herein can have at least about 20, at least about 40, at least about 60, at least about 80, at least about 100, at least about 120, at least about 140, at least about 160, at least about 180, at least about 200, at least about 220, at least about 240, at least about 260, at least about 280, at least about 300, at least about 320, at least about 340, at least about 360, at least about 380, at least about 400, at least about 420, at least about 440, at least about 460, at least about 480, at least about 500, at least about 520, at least about 540, at least about 560, at least about 580, at least about 600, at least about 620, at least about 640, at least about 680, at least about 700, at least about 720, at least about 740, at least about 760, at least about 780, at least about 800, at least about 820, at least about 840, at least about 860, at least about 880, at least about 900, at least about 920, at least about 940, at least about 960, at least about 980, at least about 1000, at least about 1020, at least about 1040, at least about 1060, at least about 1080, at least about 1100, at least about 1120, at least about 1140, at least about 1160, at least about 1180, at least about 1200, at least about 1220, at least about 1240, at least about 1260, at least about 1280, at least about 1300, at least about 1320, at least about 1340, at least about 1360, at least about 1380, at least about 1400, at least about 1420, at least about 1440, at least about 1460, at least about 1480, at least about 1500, at least about 1520, at least about 1540, at least about 1560, at least about 1580, at least about 1600, at least about 1620, at least about 1640, at least about 1660, at least about 1680, at least about 1700, at least about 1720, at least about 1740, at least about 1760, at least about 1780, at least about 1800, at least about 1820, at least about 1840, at least about 1860, at least about 1880, at least about 2000, at least about 2020, at least about 2040, at least about 2060, at least about 2080, at least about 2100, at least about 2120, at least about 2140, at least about 2160, at least about 2180, at least about 2200, at least about 2220, at least about 2240, at least about 2260, at least about 2280, at least about 2300, at least about 2320, at least about 2340, at least about 2360, at least about 2380, at least about 2400, at least about 2420, at least about 2440, at least about 2460, at least about 2480, at least about 2500, at least about 2520, at least about 2540, at least about 2560, at least about 2580, at least about 2600, at least about 2620, at least about 2640, at least about 2660, at least about 2680, at least about 2700, at least about 2720, at least about 2740, at least about 2760, at least about 2780, at least about 2800, at least about 2820, at least about 2840, at least about 2860, at least about 2880, at least about 2900, at least about 2920, at least about 2940, at least about 2960, at least about 2980, at least about 3000, at least about 3020, at least about 3040, at least about 3060, at least about 3080, at least about 3100, at least about 3120, at least about 3140, at least about 3160, at least about 3180, at least about 3200, at least about 3220, or at least about 3240 consecutive nucleotides of any of the nucleic acid sequences disclosed herein, or of any reverse complements of any of the nucleic acid sequences disclosed herein.

Although methods and materials similar or equivalent to those described herein can be used in the practice or testing of the present disclosure, suitable methods, and materials are described below.

### Synthetic Promoter Strategy and Design

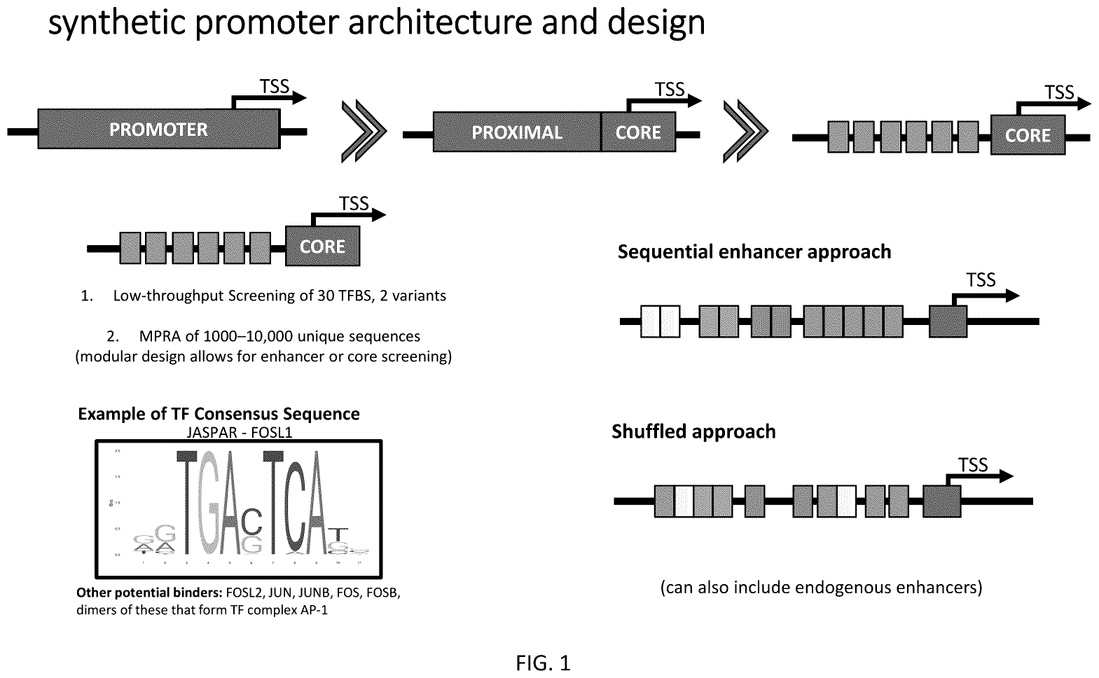

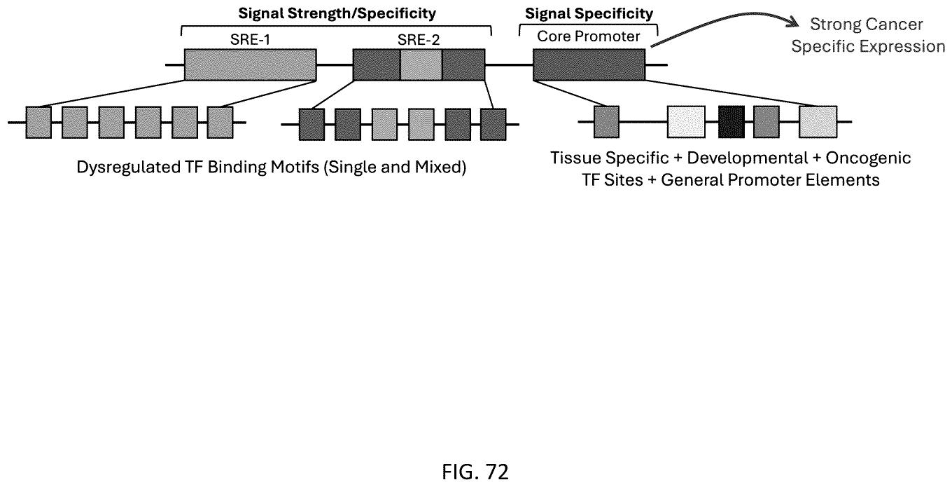

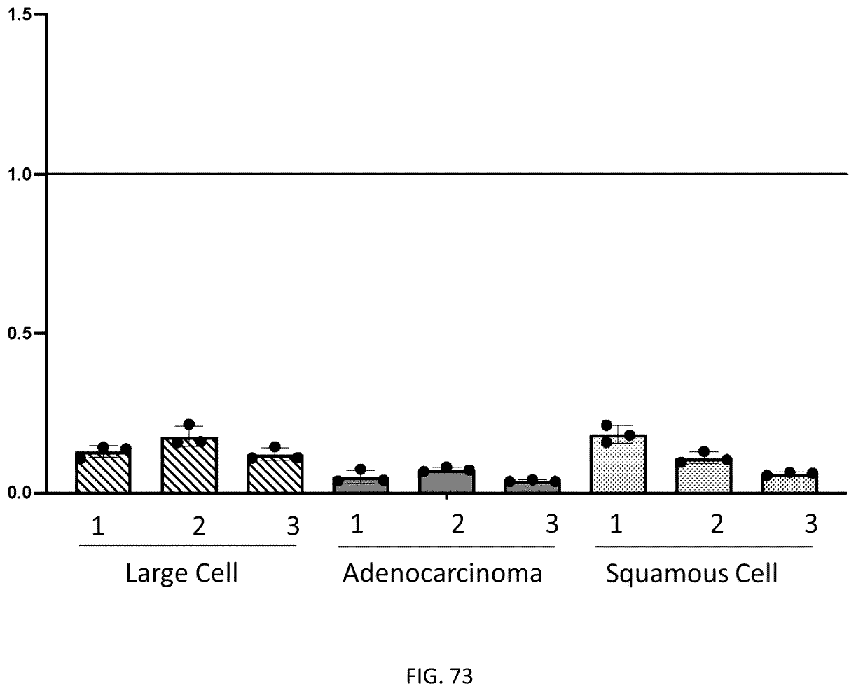

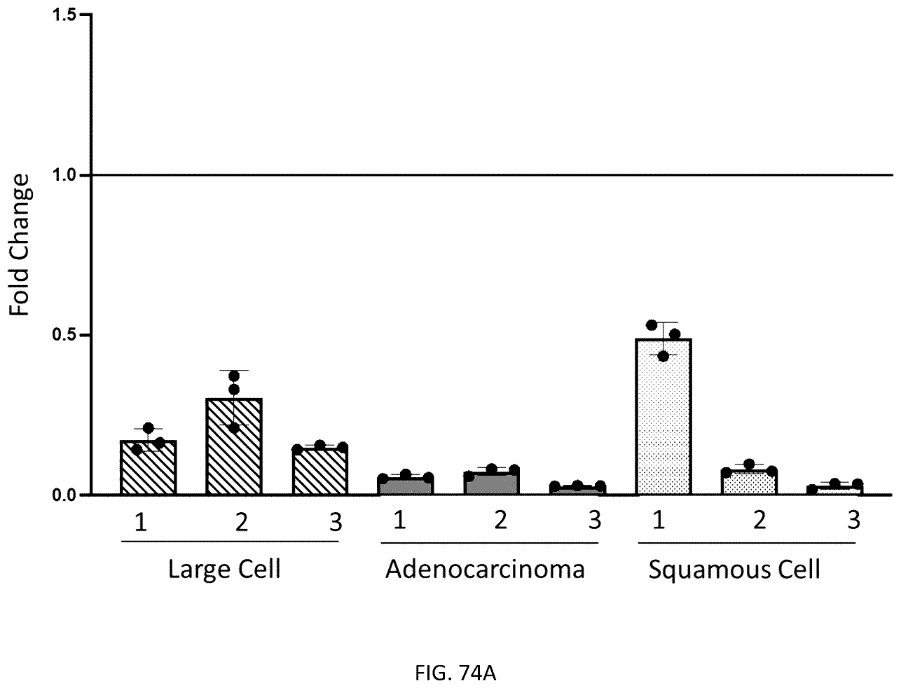

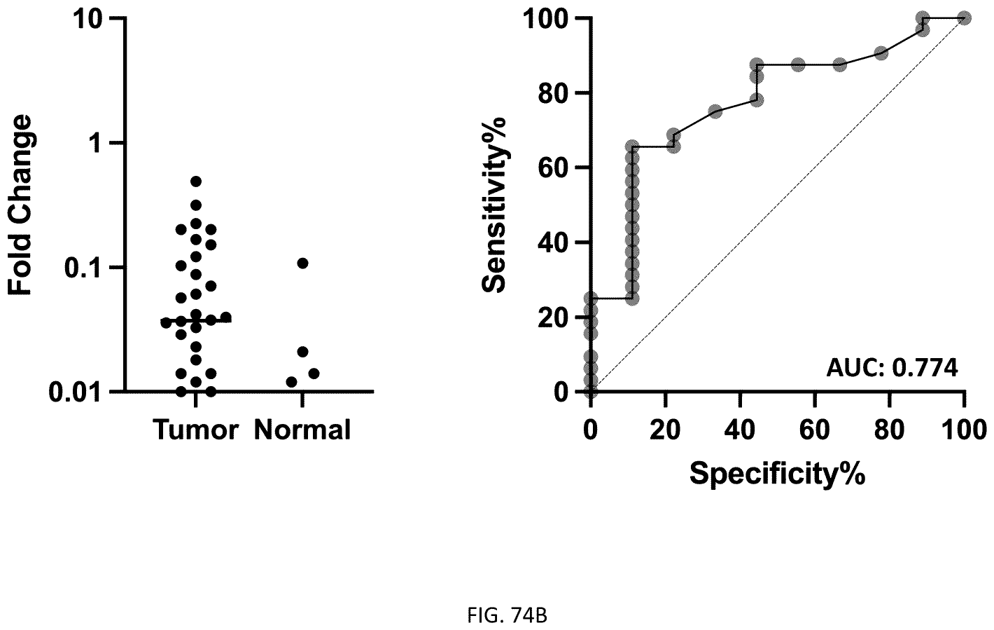

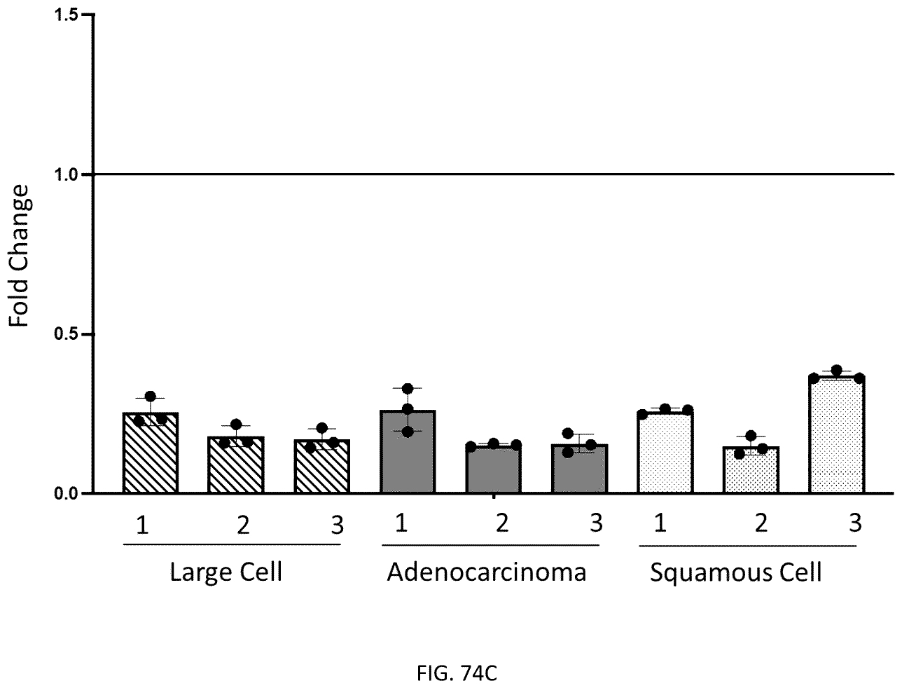

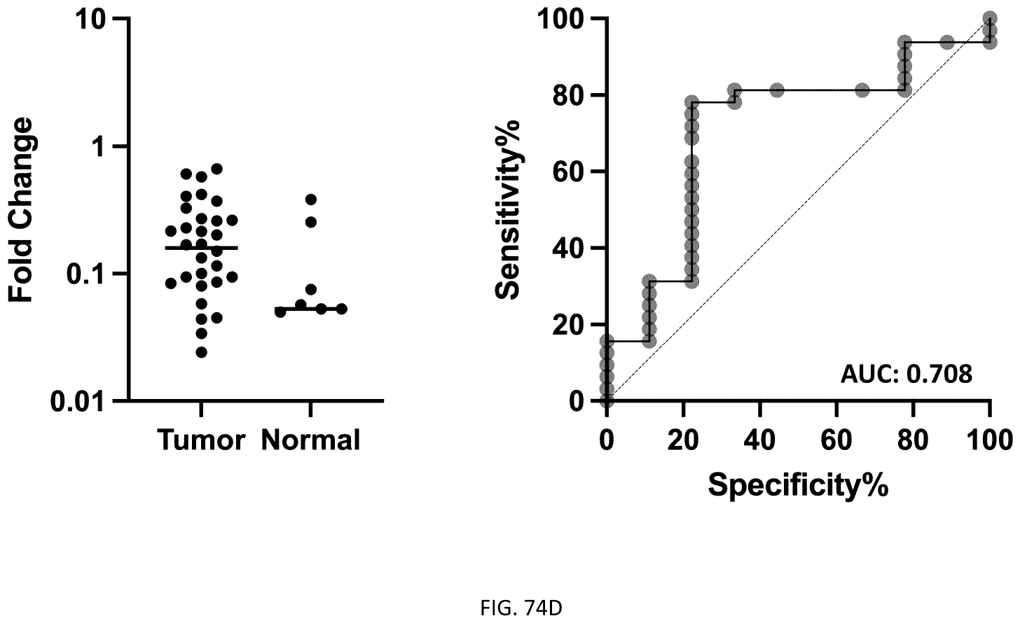

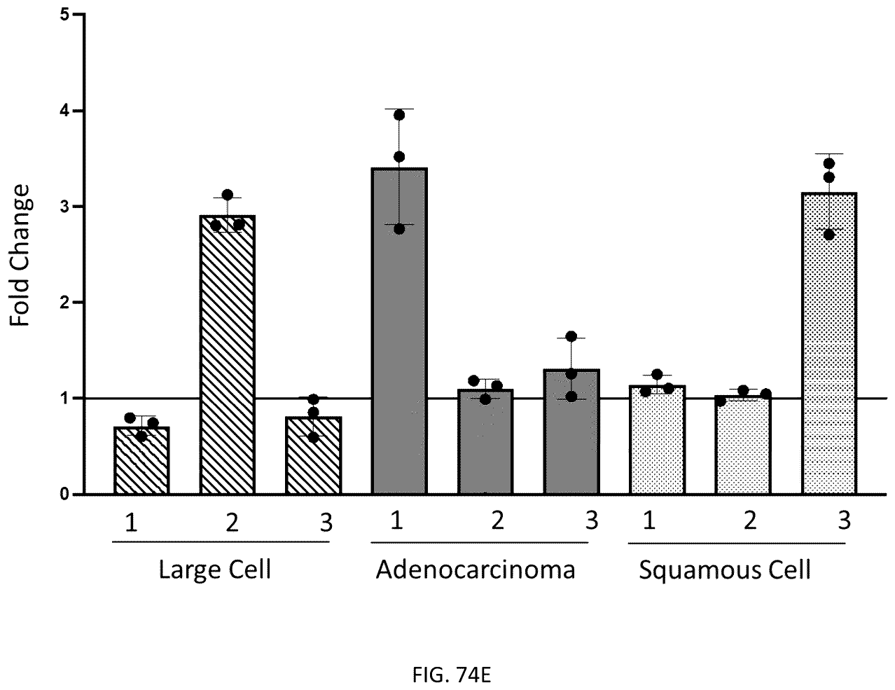

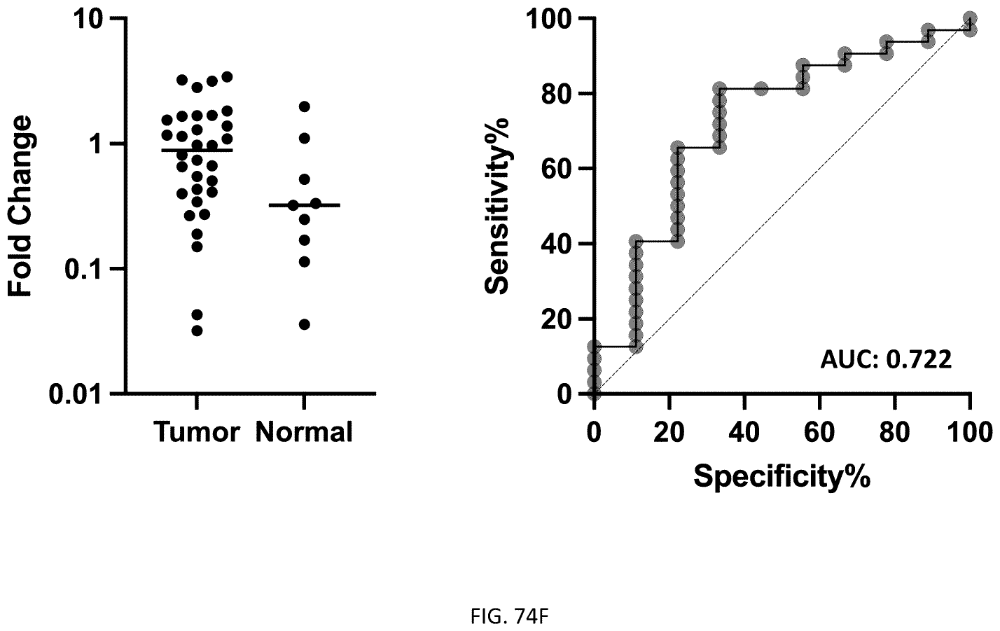

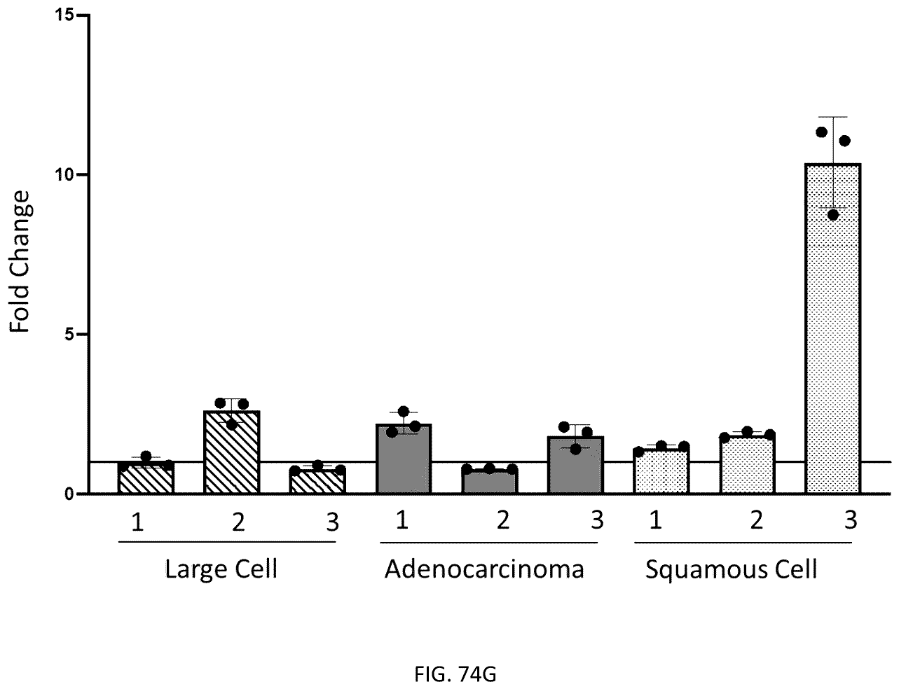

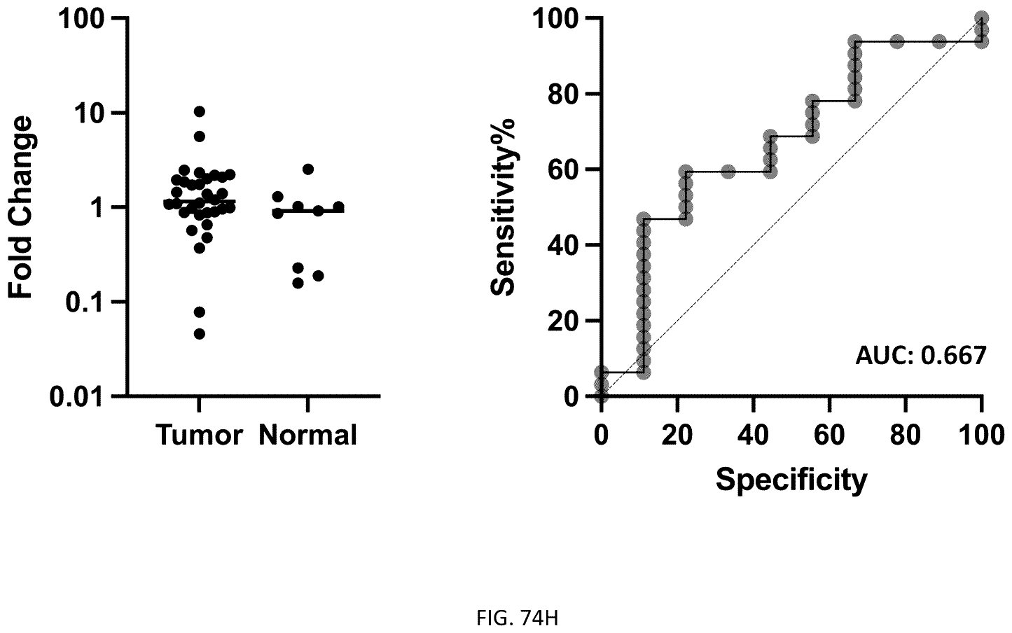

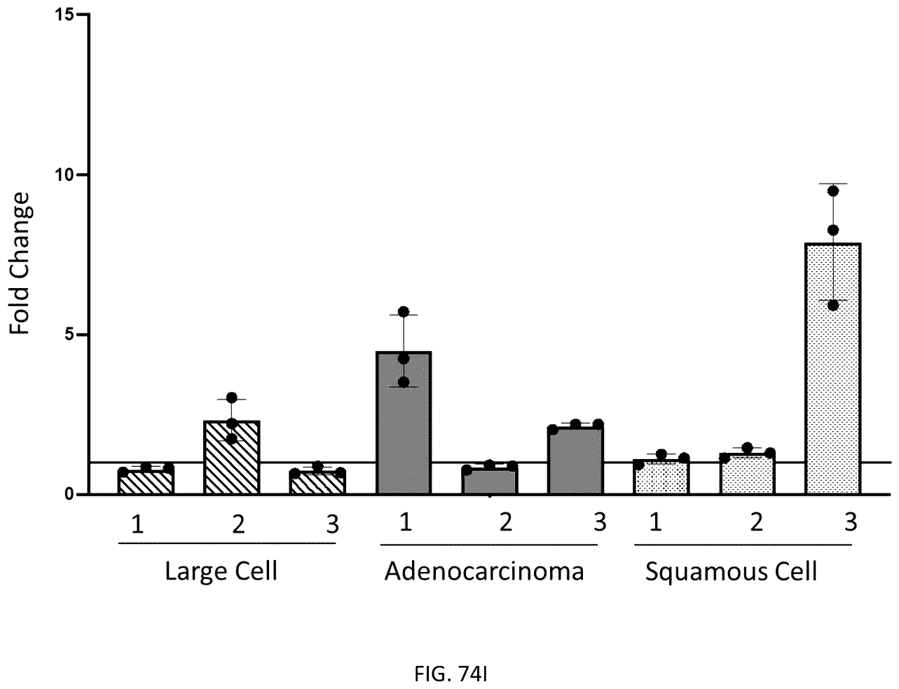

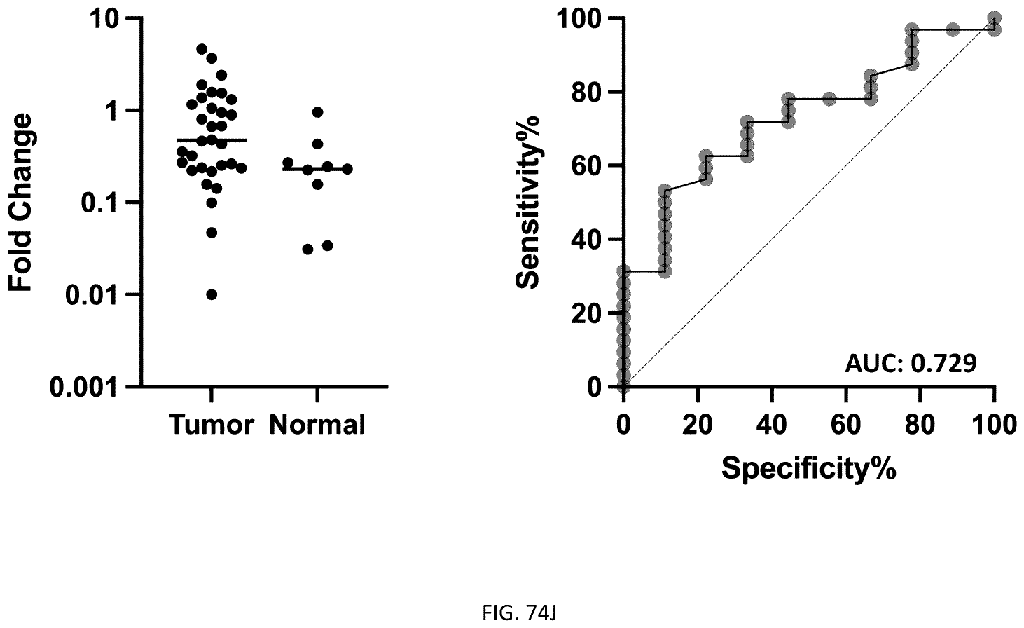

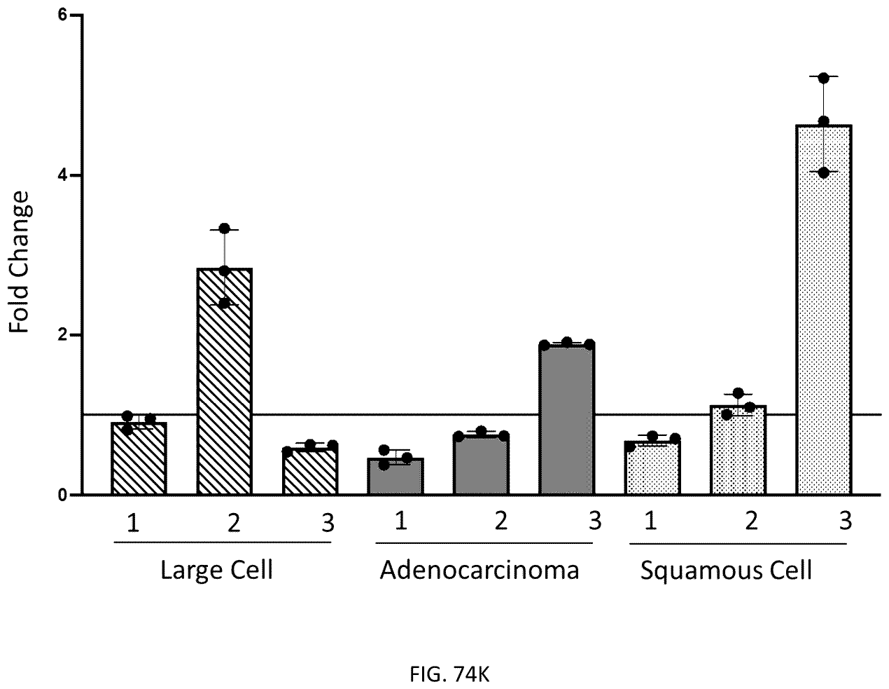

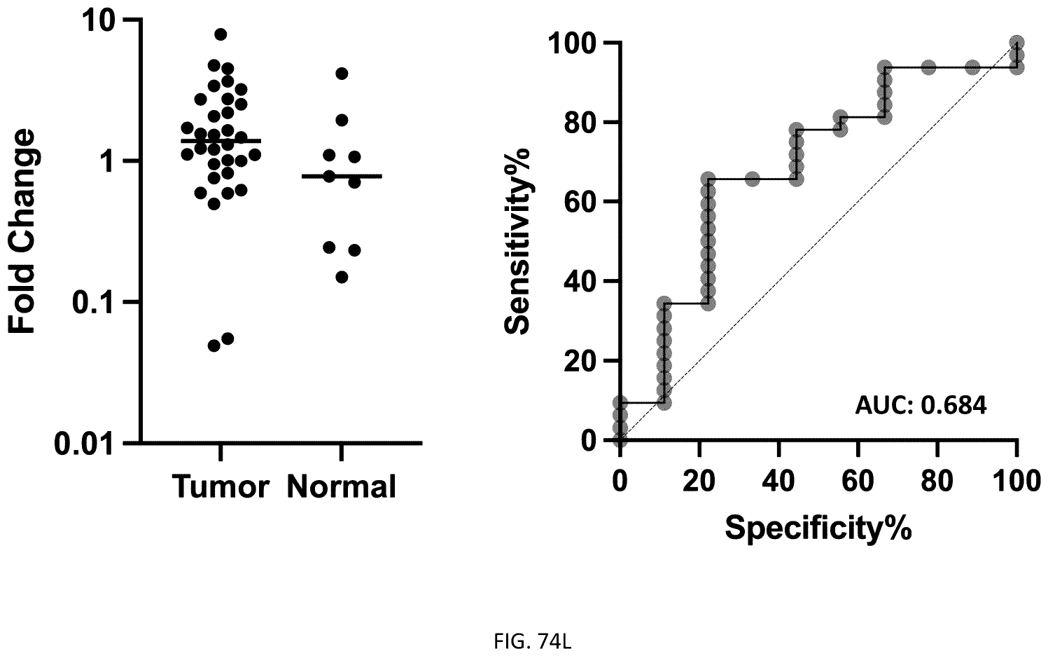

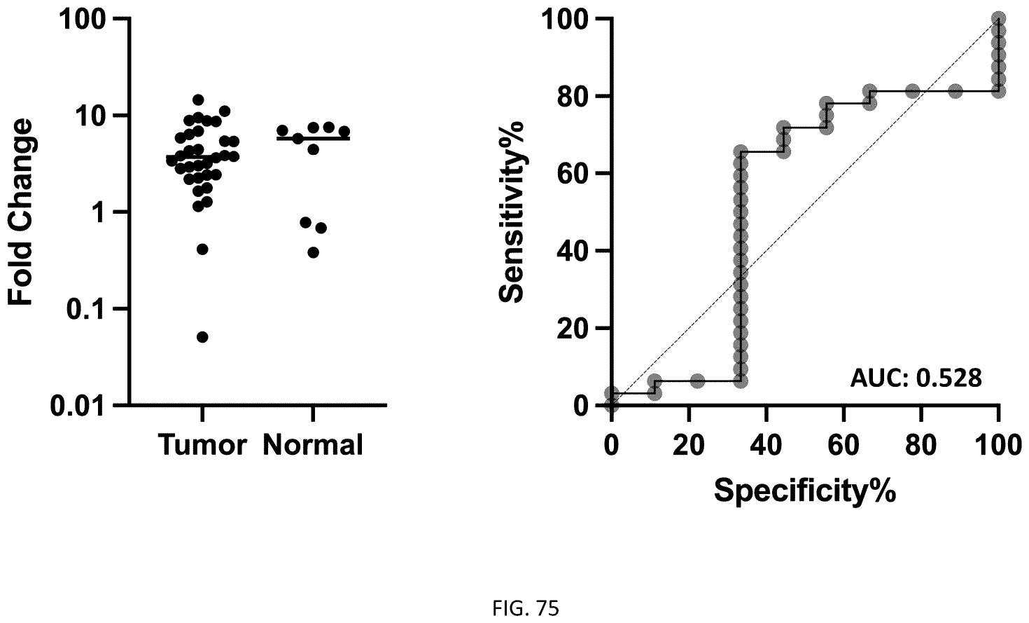

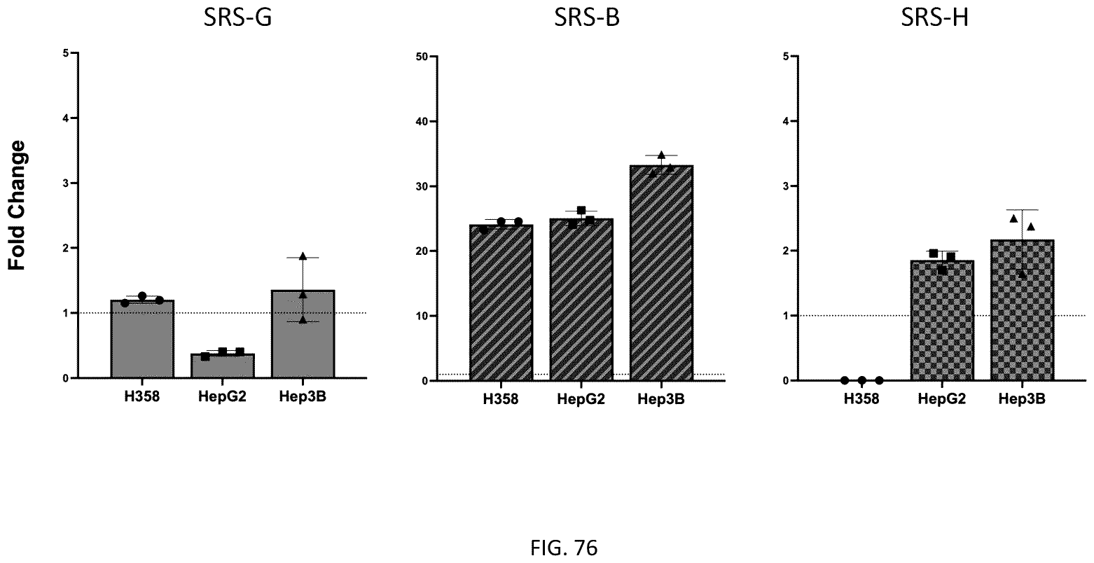

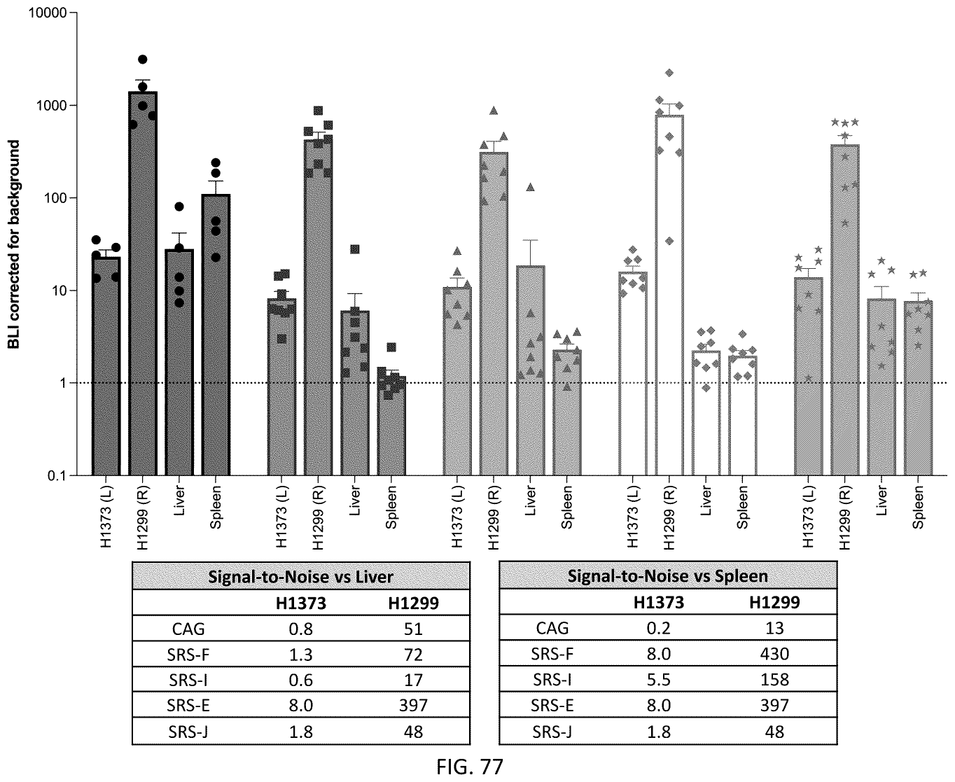

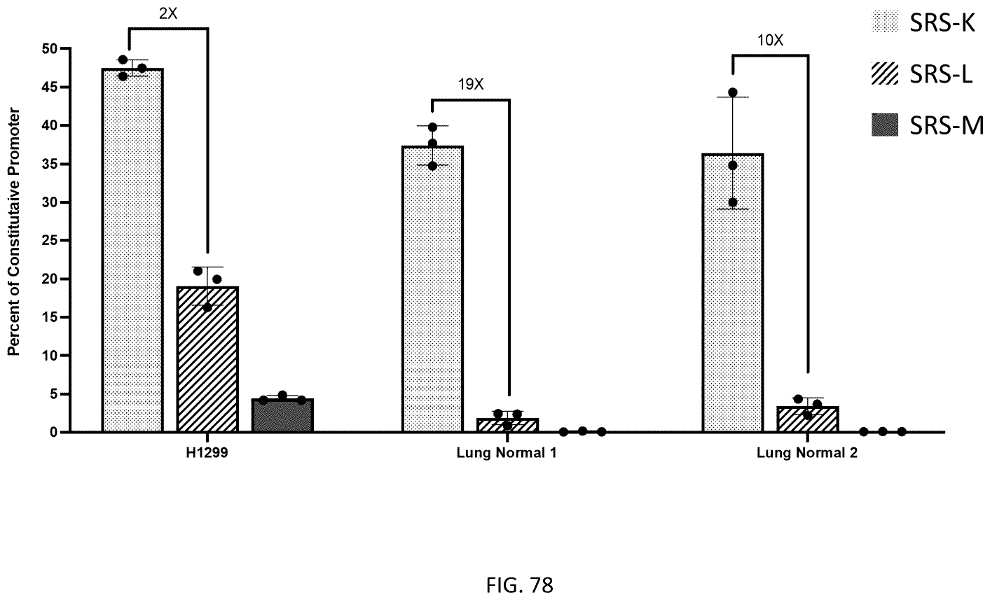

Provided herein are synthetic promoters that can be activated in target cells with high sensitivity and specificity. These promoters can be modular and engineerable. In some embodiments, synthetic promoters described herein can be designed to drive specificity and sensitivity. For example, synthetic promoters can be designed to specifically respond to dysregulated pathways in cancer. In one embodiment, synthetic promoters described herein can comprise an endogenous promoter of a gene that is expressed specifically or preferentially in cancer cells compared to non-cancer cells. In another embodiment, synthetic promoters described herein can comprise a core promoter. A core promoter described herein can comprise a minimal promoter sequence of an endogenous promoter of a gene expressed specifically or preferentially in cancer cells compared to non-cancer cells. A minimal promoter can refer to a short DNA sequence that can allow for the formation of a transcription initiation complex or a DNA sequence comprising a minimal number of nucleotides sufficient to allow for the formation of a transcription initiation complex. In some embodiments, synthetic promoters described herein can comprise a structure comprising three major components (1) a cancer-specific promoter or core promoter, (2) cancer-activated response elements (e.g., binding sites of one or more transcription factors specific for cancer cells), and optionally (3) an enhancer to boost signal strength (e.g., see FIG. 1 or FIG. 72). In some embodiments, synthetic promoters described herein can comprise only (1) a cancer-specific promoter or core promoter. In some embodiments, synthetic promoters described herein can comprise only (1) a cancer-specific promoter or core promoter and (3) an enhancer to boost signal strength. In some embodiments, an enhancer or a transcription binding site can be referred to as a Synthetic Response Element (SRE). In some embodiments, a synthetic promoter comprising a promoter or core promoter and one or more SREs can be referred to as a Synthetic Response Sensor (SRS). In some embodiments, cancer-activated response elements can be designed and constructed to respond to specific dysregulated transcription factors. In some embodiments, cancer-activated response elements described herein can demonstrate predictable activity based on transcriptomic and proteomic data when applied in new cancer models.

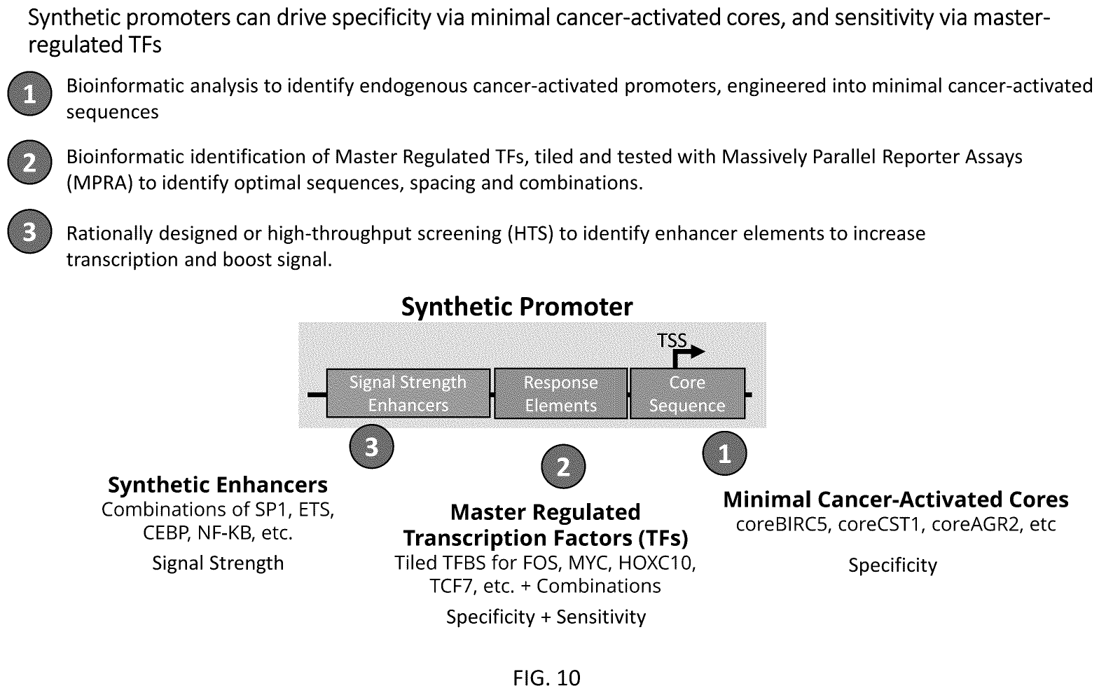

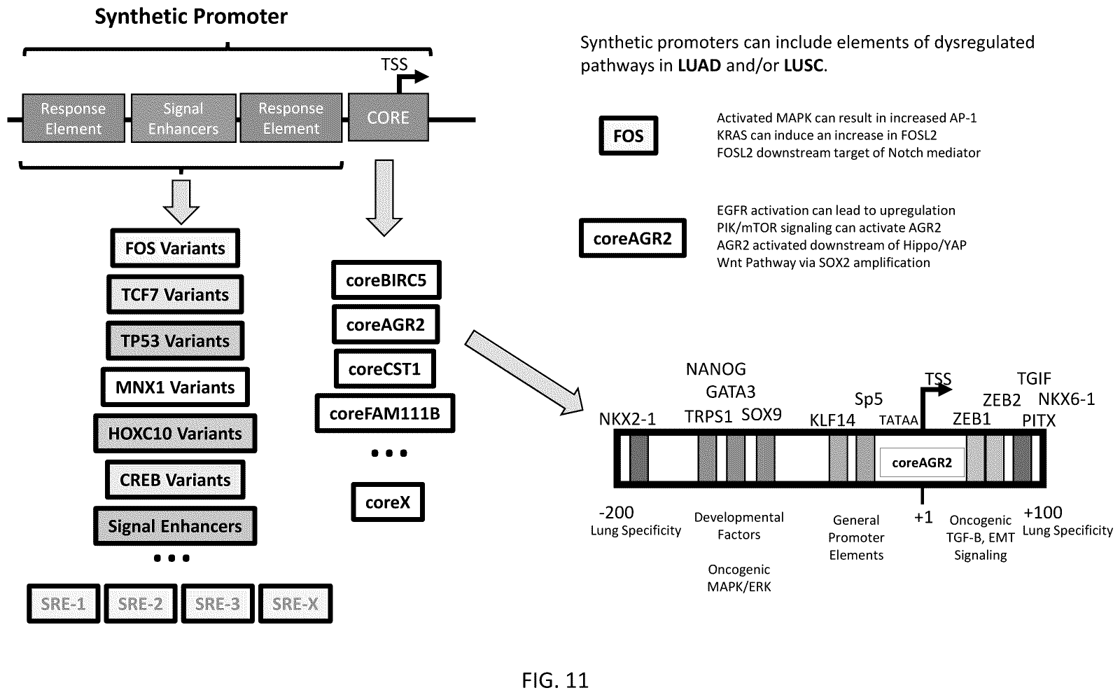

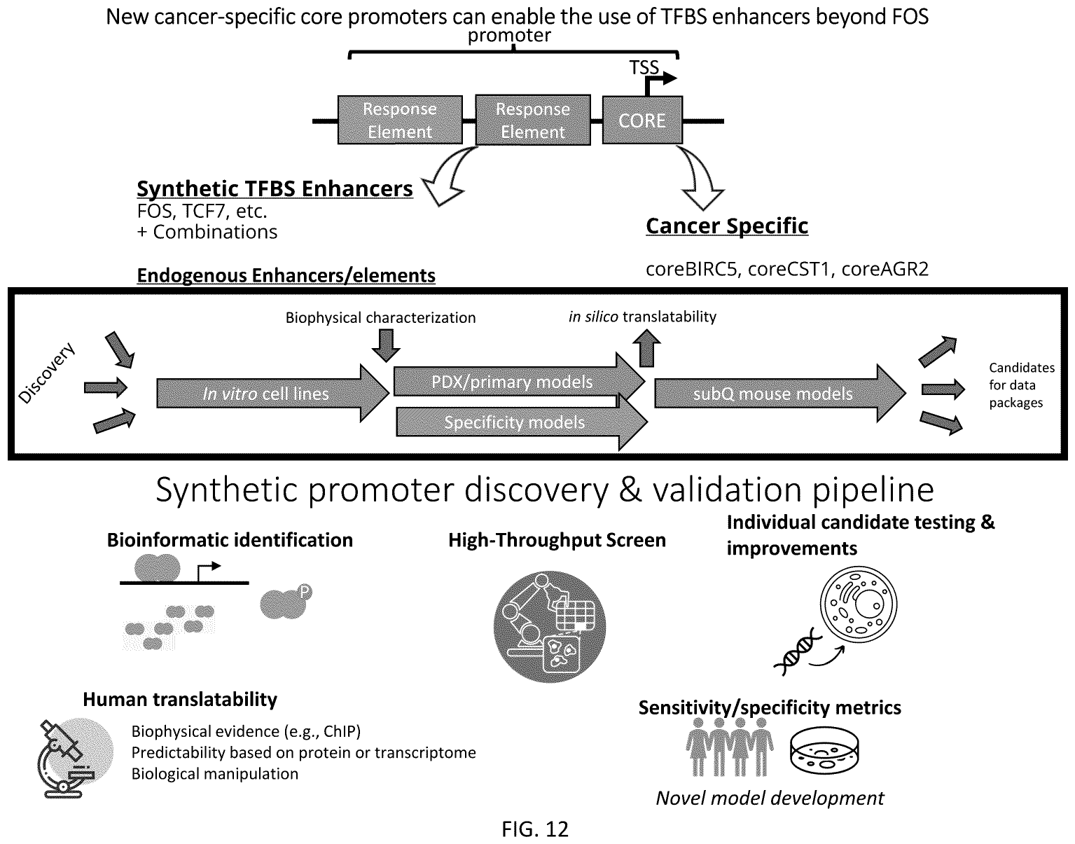

In some embodiments, bioinformatics can be used to identify endogenous cancer-activated core promoter sequences. In some embodiments, multi-omic approaches can be used to identify transcription factors (TFs) and their binding sites that are master-regulated. In some embodiments, such TF binding sites can be tiled and tested using high-throughput sequencing (HTS) to optimize promoter sequences, spacing, and combinations thereof. In some embodiments, one or more rationally designed enhancer elements that increase transcription and boost reporter signal can be used. An exemplary workflow and synthetic promoter are described in FIGS. 10-13.

In some embodiments, candidate TF binding site sequences can be identified using Multi-Omics Factor Analysis (MOFA). In some embodiments, candidate TF binding site sequences can be highly dysregulated. In some embodiments, Multi-Omics Factor Analysis (MOFA) can be used to identify TFs specific for a cancer. In some embodiments, a cancer can comprise lung cancer, breast cancer, liver cancer, and/or colorectal cancer. In some embodiments, a lung cancer can comprise non-small cell lung cancer (NSCLC).

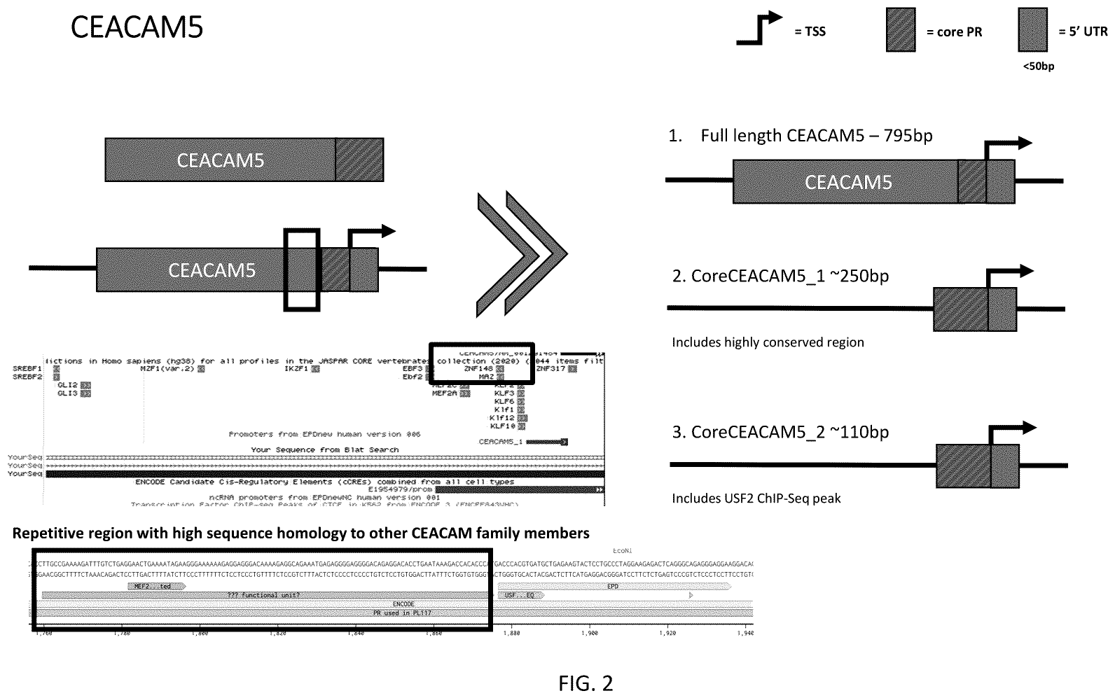

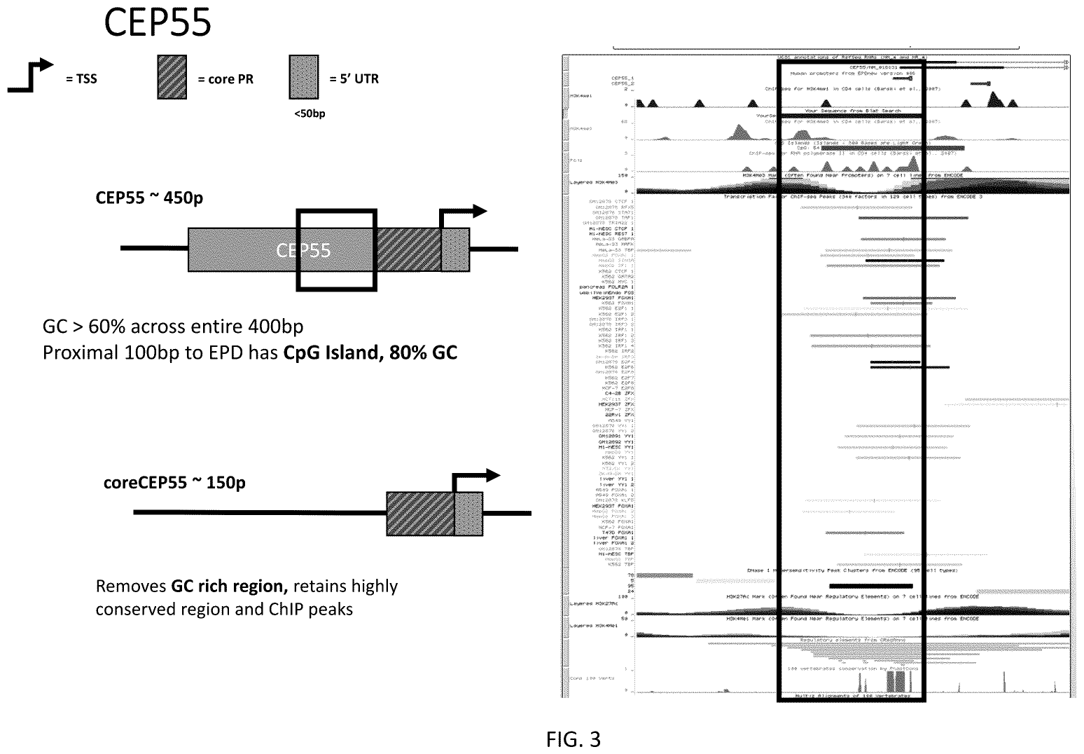

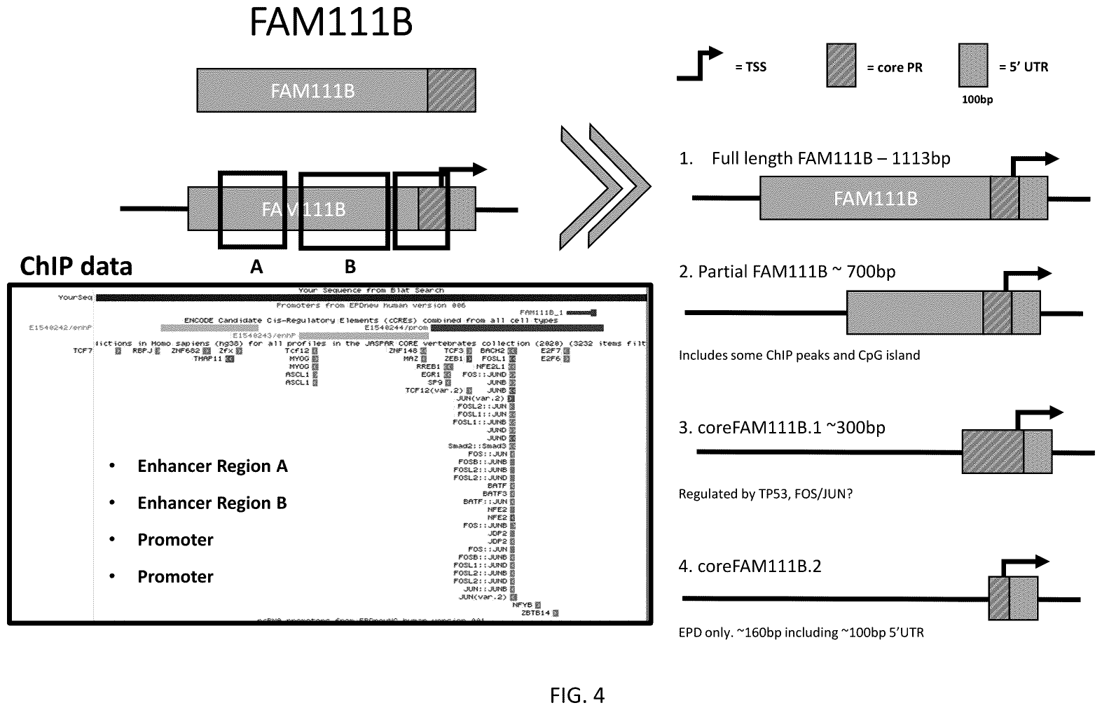

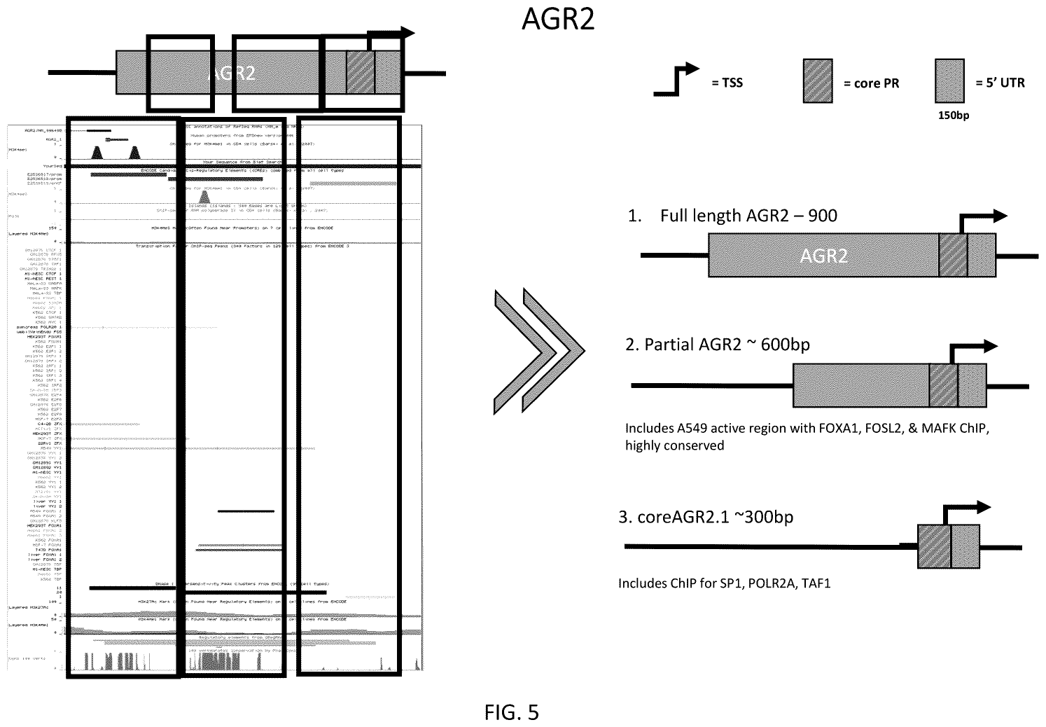

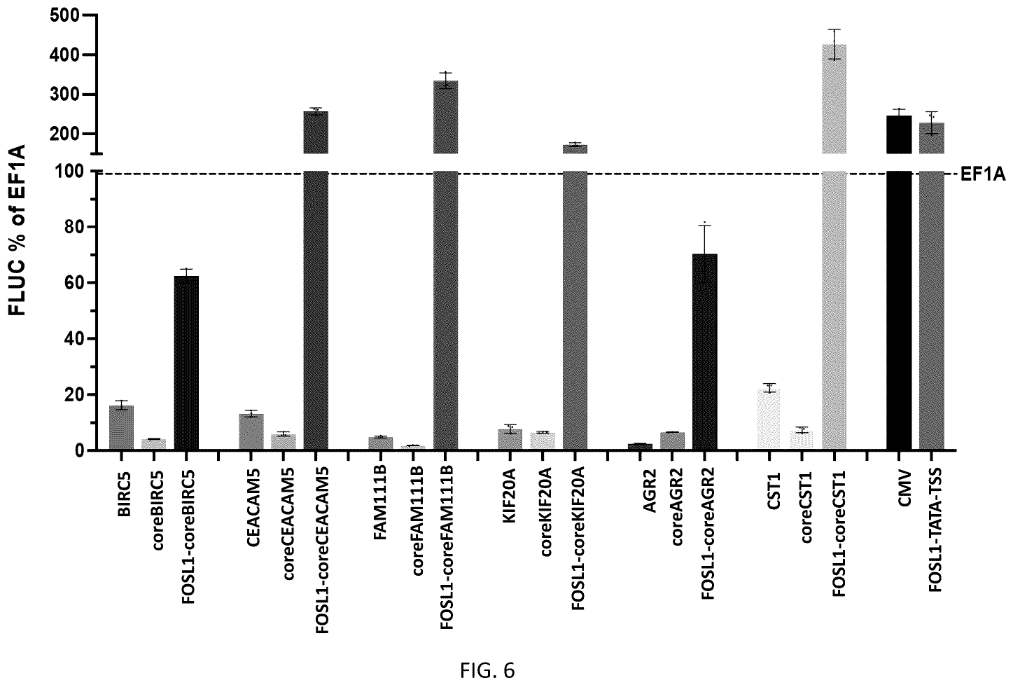

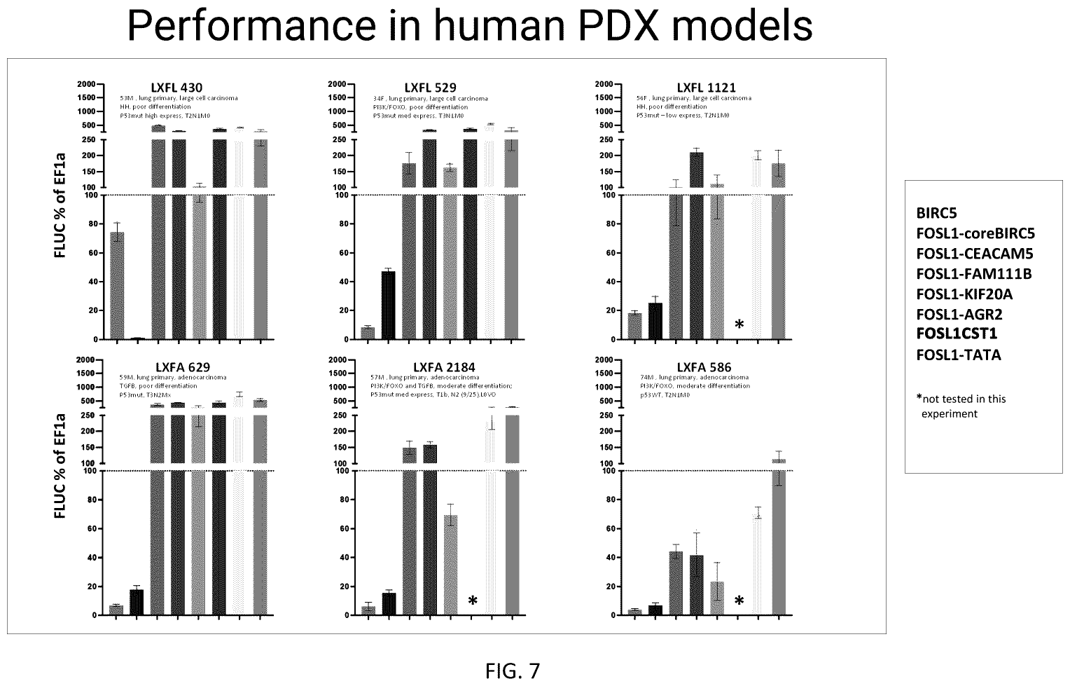

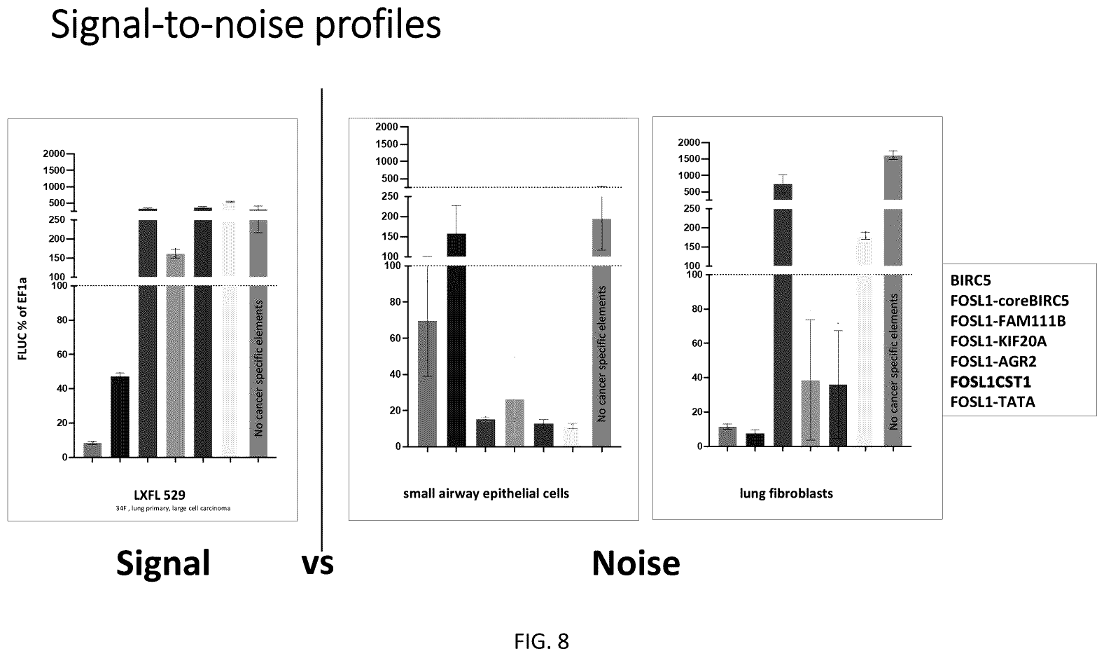

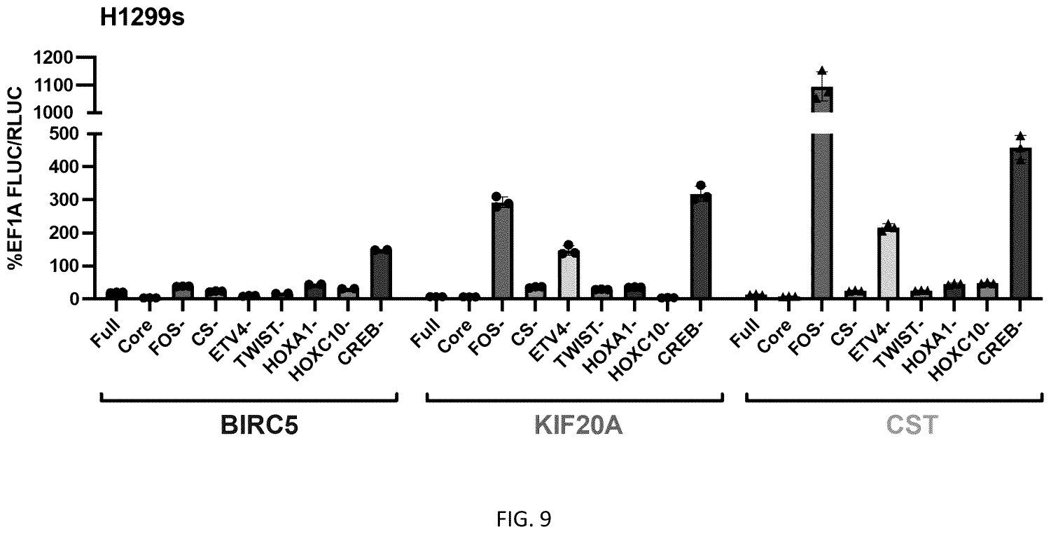

In some embodiments, a synthetic promoter can comprise a core promoter sequence. In some embodiments, a core promoter can be identified by analyzing one or more endogenous promoters that can drive cancer specific expression in vitro and/or in vivo, that is the one or more endogenous promoters can preferentially activate gene expression of a gene that is functionally or operatively linked to said one or more promotors in cancer cells (e.g., either in a subject or cancer cell lines) compared to corresponding healthy or normal cells. In some embodiments, one or more endogenous promoters can be analyzed and annotated using UCSC genome browser to build and test core promoters. In some embodiments, core promoters identified can be combined with other elements described herein. In some embodiments, a core promoter sequence can comprise a minimal cancer-activated core promoters. For example, a core promoter sequence can comprise a promoter sequence comprising a minimal number of nucleotides sufficient to drive expression (e.g., recruit transcription initiation complex) of a gene that is functionally or operatively linked to the core promoter in cancer cells. Examples of a minimal cancer-activated cores can include, but are not limited to, coreBIRC5, coreCST1, coreAGR2, coreFAM111B, CEACAM5, CEP55, UBE2C, FAM111B, KIF20A, FOXA1, MYC, or TP53 (e.g., FIGS. 2-5 and FIG. 11). In some embodiments, a core promoter sequence can provide specificity. In some embodiments, a synthetic promoter can comprise a response element. In some embodiments, a response element can comprise a binding site for a master regulated transcription factor (TF). Examples of a master regulated TF can include, but are not limited to, tiled TFBS for FOS, CREB, MYC, HOXC10, TCF7, or combinations thereof. In some embodiments, a response element can provide specificity and/or sensitivity. In some embodiments, a synthetic promoter can comprise a signal strength enhancer. In some embodiments, a signal strength enhancer can comprise a synthetic enhancer (also referred herein as a Synthetic Response Element or SRE). Examples of a synthetic enhancer can include, but are not limited to enhancers of SP1, ETS, CEBP, NF-KB, or combinations thereof. In some embodiments, a synthetic enhancer can provide signal strength. Table A shows a table comparing different synthetic promoters. In some embodiments, synthetic promoters (FOS-AGR2, FOS-CST1, and HIGH-FAM111B) can drive high expression of the reporter gene and have improved signal-to-noise ratio (SNR) compared to BIRC5 variant promoters.

TABLE A
Exemplary Synthetic Promoters

| | | | | H1299 SubQ | H1299 SubQ |
|---|---|---|---|---|---|
| | In Vitro | In Vitro | H1299 SubQ | Tumor SNR | Tumor SNR |
| Promoter | Signal | Noise | Tumor Signal | Lung | Liver |
| CAG | +++ | −−− | 38/11 | 10/3 | <<1 |
| FOS-TATA | +++ | −−− | 9 | 3.6 | <<1 |
| BIRC5 | + | −− | | n/a at 1.4 mpk | |
| FOSL- | ++ | −− | | n/a at 1.4 mpk | |
| coreBIRC5 | | | | | |
| HIGH- | +++ | −− | 3.6 | 3.2 | 1.8 |
| coreBIRC5 | | | | | |
| FOS- | +++ | −− | 9.3/3 | 10/3.3 | 3.2 |
| coreAGR2 | | | 3.8 | 5 | 2.5 |
| FOS- | +++ | − | 3.7 | 4.1 | 1 |
| coreCST1 | | | | | |
| HIGH- | +++ | −− | 7.5 | 3.4 | 1.33 |
| coreFAM111B | | | | | |

In some embodiments, synthetic promoters described herein that can drive expression in a broad range of cancer cells or cancer tissues including, but not limited to, lung cancer cells, can be identified using methods described herein. In one example, promoters identified using methods described herein can include promoters or binding sites/motifs of TCF7, one of TCFs that can be activated by Wnt/B-cat pathway, known for functioning in development pathways. In some embodiments, cancer cell lines based on Wnt/B-cat pathway can be used for further analysis. For example, a principal component analysis (PCA) of PDX database and CCLE focused on the B-cat/Wnt pathway can be used to choose cell lines for further analysis (e.g., 163 genes involved in Wnt/B-cat pathway, 50 CCLE lung cell lines, and 91 PDX lung cell lines). In some embodiments, a PCA including all lung-related PDXs from CRL as well as the CCLE transcriptome database can be used. Examples of cell lines include, but are not limited to, PC2, H520, LK2, or PDX430. In some embodiments, these cell lines can have similar level of expressions of Wnt7B, CCND1, FZD3, AXIN2 or NKD1. In another example, promoters identified using methods described herein can include promoters of TP53, a tumor suppressor that can activate or repress expression depending on location of the binding site. In some embodiments, TP53 binding sequence or motifs can be included in a promoter or a core promoter.

In some embodiments, synthetic promoters that can integrate multiple signaling can be engineered using methods described herein. For example, binding sequences or motifs of TCF, TP53, FOS, MNX1, HOXC10, of CREB can be combined with core promoters described herein to engineer synthetic promoters. In some embodiments, synthetic promoters can comprise promoters or binding sequences/motifs/sites TFs of genes in multiple regulatory pathways. In some embodiments, synthetic promoters comprising two or more endogenous or core promoters can result in gene expression with greater signal and coverage. Details of synthetic promoter design and construction are described in Example 1 and Example 2.

### Synthetic Response Sensor (SRSs or Synthetic Promoter) and Synthetic Response Elements (SREs)

In some aspects, provided herein is a recombinant polynucleotide comprising a Synthetic Response Sensor (SRS) that can drive expression of a gene or an ORF operatively linked to the SRS in tissue- or cell-specific manner. In some embodiments, an SRS described herein can drive cancer specific or cancer-activated expression of a gene or an ORF operatively linked to the SRS. For example, an SRS described herein can drive expression of a gene or an ORF operatively linked to the SRS preferentially or specifically in cancer cells or cancer tissues compared to non-cancer cells or non-cancer tissues. In some embodiments, the expression level of a gene or an ORF operatively linked to an SRS is higher in cancer cells or cancer tissues compared to non-cancer cells or non-cancer tissues. In some embodiments, an SRS can comprise a promoter or a core promoter and one or more Synthetic Response Elements (SREs). In some embodiments, the promoter or the core promoter can provide tissue- or cell-specificity for gene expression. In some embodiments, an SRE can provide tissue- or cell-specificity for gene expression and/or enhance the tissue- or cell-specificity of gene expression. In some embodiments, an SRE can comprise a plurality of binding sites for one or more transcription factors or a plurality of enhancers. For example, an SRE can comprise a plurality of binding sites for one or more transcription factors that are activated in cancer cells or cancer pathways or are dysregulated (e.g., expressed in aberrantly higher levels, etc.) in cancer cells or cancer pathways. In some embodiments, an SRS can drive expression of an ORF operatively linked to the SRS in cancer cells or cancer tissues but not in normal cells or tissues (including normal tissues or cells adjacent to cancer cells or cancer tissues) and/or benign lesions.

In some embodiments, an SRS can comprise a promoter and one or more SREs comprising a plurality of binding sites for one or more transcription factors and a plurality of enhancers. In some embodiments, an SRS can comprise a promoter and one or more SREs comprising a plurality of binding sites for one or more transcription factors. In some embodiments, an SRS can comprise a core promoter and one or more SREs comprising a plurality of binding sites for one or more transcription factors. In some embodiments, an SRS can comprise a promoter and one or more SREs comprising a plurality of enhancers. In some embodiments, an SRS can comprise a core promoter and one or more SREs comprising a plurality of enhancers. In some embodiments, an SRS can comprise a core promoter and one or more SREs comprising a plurality of binding sites for one or more transcription factors and a plurality of enhancers. An exemplary SRS is shown in FIG. 72. In one embodiment, an SRE can comprise a plurality of binding sites for one or more transcription factors, wherein each of the plurality of transcription binding sites can comprise the same binding site sequences or motifs (FIG. 72, left). In another embodiment, an SRE can comprise a plurality of binding sites for one or more transcription factors, wherein each of the plurality of transcription binding sites can comprise different binding site sequences or motifs. In yet another embodiment, an SRE can comprise a plurality of binding sites for one or more transcription factors, wherein the plurality of transcription binding sites can comprise a mixture of the same binding site sequences and different binding site sequences (FIG. 72, middle). In some embodiments, an SRS comprising an SRE that comprises a mixture of different transcription factor binding sequences or motifs can drive stronger or higher expression of an ORF operatively linked to the SRS in cancer cells or cancer tissues compared to a corresponding SRS comprising an SRE that that comprises a plurality of the same transcription binding sequences or motifs.

In some embodiments, an SRS can comprise one or more SREs comprising a plurality of binding sites for one or more transcription factors at the 5′ or upstream of a promoter or a core promoter. In some embodiments, an SRS can comprise one or more SREs comprising a plurality of enhancers at the 5′ or upstream of a promoter or a core promoter. In some embodiments, an SRS can comprise a plurality of enhancers at the 5′ or upstream of a plurality of binding sites for one or more transcription factors, wherein the plurality of binding sites for one or more transcription factors are at the 5′ or upstream of a promoter or a core promoter. For example, an SRS can comprise (i) a plurality of enhancers, (ii) a plurality of binding sites for one or more transcription factors, and (iii) a promoter or a core promotor in 5′ to 3′ direction. In some embodiments, an SRS can comprise a plurality of enhancers at the 5′ or upstream of a promoter or a core promoter and at the 3′ or downstream of a plurality of binding sites for one or more transcription factors. For example, an SRS can comprise (i) a plurality of binding sites for one or more transcription factors, (ii) a plurality of enhancers, and (ii) a promoter or a core promoter in 5′ to 3′ direction.

In some embodiments, an SRS described herein can drive the expression of an ORF operably linked to the SRS in one specific type of cancer cells. In some embodiments, an SRS described herein can drive the expression of an ORF operably linked to the SRS in two or more types of cancer cells.

In some embodiments, a recombinant polynucleotide comprising an SRS describe herein can drive the expression of an ORF operably linked to the SRS at a higher level compared to a corresponding recombinant polynucleotide comprising a constitutive promoter and an ORF operatively linked to the constitutive promoter. For example, a recombinant polynucleotide comprising an SRS describe herein can drive the expression of an ORF operably linked to the SRS at a level that is at least 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 110%, 920%, 930%, 940%, 950%, 960%, 970%, 980%, 990%, or at least 1000% higher compared to a corresponding recombinant polynucleotide comprising a constitutive promoter and an ORF operatively linked to the constitutive promoter. In some embodiments, an ORF can comprise an ORF of a natural gene or a synthetic gene. In some embodiments, a natural gene or a synthetic can comprise a gene encoding a reporter protein, a biomarker protein, or a therapeutic protein.

In some embodiments, a recombinant polynucleotide comprising an SRS describe herein can drive the expression of an ORF operably linked to the SRS at a higher level in cancer cells compared to a corresponding recombinant polynucleotide comprising a constitutive promoter and an ORF operatively linked to the constitutive promoter. For example, a recombinant polynucleotide comprising an SRS describe herein can drive the expression of an ORF operably linked to the SRS in cancer cells at a level that is at least 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 110%, 920%, 930%, 940%, 950%, 960%, 970%, 980%, 990%, or at least 1000% higher compared to a corresponding recombinant polynucleotide comprising a constitutive promoter and an ORF operatively linked to the constitutive promoter.

### Promoter/Core Promoter

A core promoter described herein can comprise a minimal promoter that can comprise a transcription start site or a transcription start site sequence that is derived from a promoter of one or more genes expressed in cancer cells or cancer tissues (also referred to as a cancer-responsive gene herein). In some embodiments, a core promoter described herein can comprise a minimal promoter that can comprise a transcription start site or a transcription start site sequence that is derived from a promoter of one or more genes expressed at a higher level in cancer cells or cancer tissues compared to non-cancer cells or non-cancer tissues. For example, a core promoter described herein can comprise a minimal promoter that can comprise a transcription start site or a transcription start site sequence that is derived from a promoter of one or more genes expressed at a level that is at least 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 110%, 920%, 930%, 940%, 950%, 960%, 970%, 980%, 990%, or at least 1000% higher in cancer cells or cancer tissues compared to non-cancer cells or non-cancer tissues.

In some embodiments, a core promoter can further comprise one or more promoter elements that are derived from a promoter of one or more genes expressed in cancer cells or cancer tissues. In some embodiments, a core promoter can further comprise one or more promoter elements that are derived from a promoter of one or more genes expressed at a level that is at least 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 110%, 920%, 930%, 940%, 950%, 960%, 970%, 980%, 990%, or at least 1000% higher in cancer cells or cancer tissues compared to non-cancer cells or non-cancer tissues. In some embodiments, promoter elements can include, but are not limited to, elements specific for tissue, elements specific for development or development stage, elements specific for cancer (e.g., transcription factor binding sites specific for cancer or oncogenic transcription factor binding sites), elements important for transcription (e.g., general promoter elements). In some embodiments, a core promoter can comprise two or more promoter elements that are derived from a promoter of two or more genes expressed in cancer cells or cancer tissues. For example, a core promoter can comprise two or more promoter elements that are derived from a promoter of at least two, at least three, at least four, at least five, at least six, at least seven, at least eight, at least nine, at least 10, at least 11, at least 12, at least 13, at least 14, at least 15, at least 16, at least 17, at least 18, at least 19, or at least 20 genes expressed in cancer cells or cancer tissues. Non-limiting examples of cancer-responsive genes can include TCF7, MNX1, HOXC10, TP53, CEACAM5, CEP55, FAM111B, CST1, BIRC5, AGR2, FOXA1, cMYC, FOS, TWIST1, E2F2, UBE2C, KIF20A, or ETV4.

In some embodiments, a core promoter can comprise a minimal promoter derived from one or more genes expressed in cancer cells or cancer tissues. In one example, a core promoter can comprise a minimal promoter derived from one or more cancer-responsive genes comprising TCF7, MNX1, HOXC10, TP53, CEACAM5, CEP55, FAM111B, CST1, BIRC5, AGR2, FOXA1, cMYC, FOS, TWIST1, E2F2, UBE2C, KIF20A, or ETV4. In another example, a core promoter can comprise a hybrid minimal promoter derived from two or more cancer-responsive genes comprising TCF7, MNX1, HOXC10, TP53, CEACAM5, CEP55, FAM111B, CST1, BIRC5, AGR2, FOXA1, cMYC, FOS, TWIST1, E2F2, UBE2C, KIF20A, or ETV4. In some embodiments, a core promoter can comprise a minimal promoter and one or more promoter elements described herein derived from two or more cancer-responsive genes comprising TCF7, MNX1, HOXC10, TP53, CEACAM5, CEP55, FAM111B, CST1, BIRC5, AGR2, FOXA1, cMYC, FOS, TWIST1, E2F2, UBE2C, KIF20A, or ETV4. In some embodiments, a core promoter can comprise a minimal promoter and two or more promoter elements described herein derived from TCF7 and HOXC10. In some embodiments, a core promoter can comprise a minimal promoter and two or more promoter elements described herein derived from TP53 and CEP55.

In some embodiments, a core promoter can comprise a minimal promoter and two or more promoter elements described herein derived from FAM111B and KIF20A. In some embodiments, a core promoter can comprise a minimal promoter and two or more promoter elements described herein derived from BIRC5 and E2F2. In some embodiments, a core promoter can comprise a minimal promoter and two or more promoter elements described herein derived from CEACAM5 and TWIST1. In some embodiments, a core promoter can comprise a hybrid promoter comprising two or more promoter elements described herein derived from two or more cancer-responsive genes comprising TCF7, MNX1, HOXC10, TP53, CEACAM5, CEP55, FAM111B, CST1, BIRC5, AGR2, FOXA1, cMYC, FOS, TWIST1, E2F2, UBE2C, KIF20A, or ETV4. In some embodiments, a core promoter can comprise a hybrid promoter comprising two or more promoter elements described herein derived from TCF7 and HOXC10. In some embodiments, a core promoter can comprise a hybrid promoter comprising two or more promoter elements described herein derived from TP53 and CEP55. In some embodiments, a core promoter can comprise a hybrid promoter comprising two or more promoter elements described herein derived from FAM111B and KIF20A. In some embodiments, a core promoter can comprise a hybrid promoter comprising two or more promoter elements described herein derived from BIRC5 and E2F2. In some embodiments, a core promoter can comprise a hybrid promoter comprising two or more promoter elements described herein derived from CEACAM5 and TWIST1. In some embodiments, a core promoter can comprise a hybrid promoter comprising a minimal promoter and two or more promoter elements described herein derived from two or more cancer-responsive genes comprising TCF7, MNX1, HOXC10, TP53, CEACAM5, CEP55, FAM111B, CST1, BIRC5, AGR2, FOXA1, cMYC, FOS, TWIST1, E2F2, UBE2C, KIF20A, or ETV4. In some embodiments, a core promoter can comprise a hybrid promoter comprising a minimal promoter and two or more promoter elements described herein derived from TCF7 and HOXC10. In some embodiments, a core promoter can comprise a hybrid promoter comprising a minimal promoter and two or more promoter elements described herein derived from TP53 and CEP55. In some embodiments, a core promoter can comprise a hybrid promoter comprising a minimal promoter and two or more promoter elements described herein derived from FAM111B and KIF20A. In some embodiments, a core promoter can comprise a hybrid promoter comprising a minimal promoter and two or more promoter elements described herein derived from BIRC5 and E2F2. In some embodiments, a core promoter can comprise a hybrid promoter comprising a minimal promoter and two or more promoter elements described herein derived from CEACAM5 and TWIST1.

In some embodiments, a core promoter can comprise a hybrid promoter comprising a chimeric sequence of two or more promoter elements from two or more cancer-responsive genes comprising TCF7, MNX1, HOXC10, TP53, CEACAM5, CEP55, FAM111B, CST1, BIRC5, AGR2, FOXA1, cMYC, FOS, TWIST1, E2F2, UBE2C, KIF20A, or ETV4. In some embodiments, a core promoter can comprise a hybrid promoter comprising a chimeric sequence of two or more promoter elements derived from TCF7 and HOXC10. In some embodiments, a core promoter can comprise a hybrid promoter comprising a chimeric sequence of two or more promoter elements derived from TP53 and CEP55. In some embodiments, a core promoter can comprise a hybrid promoter comprising a chimeric sequence of two or more promoter elements derived from FAM111B and KIF20A. In some embodiments, a core promoter can comprise a hybrid promoter comprising a chimeric sequence of two or more promoter elements derived from BIRC5 and E2F2. In some embodiments, a core promoter can comprise a hybrid promoter comprising a chimeric sequence of two or more promoter elements derived from CEACAM5 and TWIST1.

In some embodiments, a core promoter can comprise a TATA box or a TATA box sequence. In some embodiments, a core promoter can comprise a sequence of a region from about −300 bp to about +100 bp, from about −250 bp to about +100 bp, from about −200 bp to about +100 bp, from about −150 bp to about +100 bp, from about −100 bp to about +100 bp, from about −90 bp to about +100 bp, from about −80 bp to about +100 bp, from about −70 bp to about +100 bp, from about −60 bp to about +100 bp, from about −50 bp to about +100 bp, from about −40 bp to about +100 bp, or from about −30 bp to about +100 bp relative to a transcription start site (TSS) of a cancer-responsive gene. In some embodiments, a core promoter can comprise a sequence of a region from about 300 bp upstream of a TSS to about 100 bp downstream of a TSS, from about 250 bp upstream of a TSS to about 100 bp downstream of a TSS, from about 200 bp upstream of a TSS to about 100 bp downstream of a TSS, from about 150 bp upstream of a TSS to about 100 bp downstream of a TSS, from about 100 bp upstream of a TSS to about 100 bp downstream of a TSS, from about 90 bp upstream of a TSS to about 100 bp downstream of a TSS, from about 80 bp upstream of a TSS to about 100 bp downstream of a TSS, from about 70 bp upstream of a TSS to about 100 bp downstream of a TSS, from about 60 bp upstream of a TSS to about 100 bp downstream of a TSS, from about 50 bp upstream of a TSS to about 100 bp downstream of a TSS, from about 40 bp upstream of a TSS to about 100 bp downstream of a TSS, or from about 30 bp upstream of a TSS to about 100 bp downstream of a TSS of a cancer-responsive gene. In some embodiments, a cancer-responsive gene can comprise a human cancer-responsive gene.

In some embodiments, the sequence of a region from about −300 bp to about +100 bp relative to a TSS (or from about 300 bp upstream of a TSS to about 100 bp downstream of a TSS) can comprise elements that are important for transcription, elements that are tissue specific, elements that are specific for certain development stage, and/or one or more binding sites for transcription factors specific for cancer (e.g., oncogenic transcription factors). In some embodiments, a promoter or a core promoter can comprise one or more elements or sequences binding to NKX2-1, NANOG, GATA3, TRPS1, SOX9, KSLF14, Sp5, ZEB1, ZEB2, TGIF, PITX, NKX6-1, THRb, ERRa, COUP-TFII, PR, Ascl2, Slug, E2A, PITX1, or NKX3.2.

In some embodiments, a promoter or a core promoter can be operably linked to an open reading frame (ORF) of a gene of interest. A gene of interest can be any gene for which expression is desired specifically in cancer cells. Non-limiting examples of a gene of interest can include a gene encoding a therapeutic protein, a gene encoding a synthetic protein, a gene encoding a marker protein (e.g., biomarker for diagnostics, etc.), or a gene encoding a reporter protein.

In some embodiments, the core promoter can be derived from a promoter of one or more genes that are expressed at a higher level in cancer cells compared to non-cancer cells. For example, the core promoter can be derived from a promoter of one or more genes that are expressed at a level that is at least 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 110%, 920%, 930%, 940%, 950%, 960%, 970%, 980%, 990%, or at least 1000% higher in cancer cells compared to non-cancer cells. In some embodiments, the core promoter can be derived from a promoter of one or more genes that are more active in cancer cells compared to non-cancer cells. For example, the core promoter can be derived from a promoter of one or more genes that are at least 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 110%, 920%, 930%, 940%, 950%, 960%, 970%, 980%, 990%, or at least 1000% more active in cancer cells compared to non-cancer cells. In some embodiments, a phosphorylation assay can be used to measure activation or activity levels of cancer-responsive genes described herein.

In some embodiments, the core promoter can be derived from one or more cancer-responsive genes that are expressed at a higher level in cancer cells compared to non-cancer cells. For example, the core promoter can be derived from one or more cancer-responsive genes that are either expressed at a level that is at least 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 110%, 920%, 930%, 940%, 950%, 960%, 970%, 980%, 990%, or at least 1000% higher in cancer cells compared to non-cancer cells. In some embodiments, the core promoter can be derived from one or more cancer-responsive genes that are more active in cancer cells compared to non-cancer cells. For example, the core promoter can be derived from one or more cancer-responsive genes that are at least 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 110%, 920%, 930%, 940%, 950%, 960%, 970%, 980%, 990%, or at least 1000% more active in cancer cells compared to non-cancer cells. In some embodiments, a phosphorylation assay can be used to measure activation or activity levels of cancer-responsive genes described herein.

### Synthetic Response Elements—Transcription Factors (TFs)

In some embodiments, an SRS can comprise one or more SREs, wherein the one or more SREs can comprise a plurality of binding sites for one or more transcription factors. In some embodiments, a plurality of binding sites (e.g., binding site DNA sequence) for one or more transcription factors can be identified from a multi-omics approach, including but not limited to, transcriptomics, proteomics, and/or phospho-proteomics to be upregulated in cancer cells or tissues compared to normal (e.g., non-cancer) cells or tissues. In some embodiments, the one or more SREs can comprise a plurality of binding sites for one or more transcription factors that are expressed in higher levels in cancer cells compared to non-cancer cells. In some embodiments, ChIP assay can be used to measure expression levels of transcription factors described herein. In some embodiments, the one or more SREs can comprise a plurality of binding sites for one or more transcription factors that are more active in cancer cells compared to non-cancer cells. For example, the one or more SREs can comprise a plurality of binding sites for one or more transcription factors that have higher level of phosphorylation in cancer cells compared to non-cancer cells. In some embodiments, a phosphorylation assay can be used to measure activation or activity levels of transcription factors described herein.

In some embodiments, an SRS comprising a promoter (or a core promoter) and a plurality of binding sites for one or more transcription factors can drive the expression of an ORF operably linked to the promoter (or the core promoter) at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, at least 3.1-fold, at least 3.2-fold, at least 3.3-fold, at least 3.4-fold, at least 3.5-fold, at least 3.6-fold, at least 3.7-fold, at least 3.8-fold, at least 3.9-fold, at least 4-fold, at least 4.1-fold, at least 4.2-fold, at least 4.3-fold, at least 4.4-fold, at least 4.5-fold, at least 4.6-fold, at least 4.7-fold, at least 4.8-fold, at least 4.9-fold, at least 5-fold, at least 10-fold, at least 20-fold, at least 30-fold, at least 40-fold, at least 50-fold, at least 60-fold, at least 70-fold, at least 80-fold, at least 90-fold, or at least 100-fold higher than the expression of a corresponding ORF driven by a promoter (or a core promoter) without the plurality of binding sites for one or more transcription factors.

In some embodiments, an SRS comprising a promoter described herein (or a core promoter described herein, e.g., a cancer-specific core promoter comprising a TATA-TSS and other elements in −300 bp to about +100 bp relative to a TSS) and a plurality of binding sites for one or more transcription factors can drive the expression of an ORF operably linked to the promoter (or the core promoter) at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, at least 3.1-fold, at least 3.2-fold, at least 3.3-fold, at least 3.4-fold, at least 3.5-fold, at least 3.6-fold, at least 3.7-fold, at least 3.8-fold, at least 3.9-fold, at least 4-fold, at least 4.1-fold, at least 4.2-fold, at least 4.3-fold, at least 4.4-fold, at least 4.5-fold, at least 4.6-fold, at least 4.7-fold, at least 4.8-fold, at least 4.9-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, at least 20-fold, at least 21-fold, at least 22-fold, at least 23-fold, at least 24-fold, at least 25-fold, at least 26-fold, at least 27-fold, at least 28-fold, at least 29-fold, at least 30-fold, at least 31-fold, at least 32-fold, at least 33-fold, at least 34-fold, at least 35-fold, at least 36-fold, at least 37-fold, at least 38-fold, at least 39-fold, at least 40-fold, at least 41-fold, at least 42-fold, at least 43-fold, at least 44-fold, at least 45-fold, at least 46-fold, at least 47-fold, at least 48-fold, at least 49-fold, at least 50-fold, at least 55-fold, at least 60-fold, at least 65-fold, at least 70-fold, at least 75-fold, at least 80-fold, at least 85-fold, at least 90-fold, at least 95-fold, or at least 100-fold higher than the expression of a corresponding ORF driven by a non-cancer specific promoter (e.g., TATA-TSS promoter only) and the plurality of binding sites for one or more transcription factors.

Non-limiting examples of transcription factors can include TRPS1, MNX1, TWIST1, ETV4, FOSL2, NFIC, EN2, TFDP1, PITX2, TCF7L1, VENTX, HOXB9, DLX1, MYCN, SIX4, TP63, SOX11, E2F8, TFDP1, SURV, TOXE1, EN1, ZBTB7B, SP3, SIX2, XBP1, HIF-1A, CREB3L1, HSF-1, MTF1, NFE2L2, USF2, TP73, POU2F2, HOXA1, FOXO1, TFAP4, BACH1, E2F4, HOXC10, KLF11, FOXM1, E2F2, E2F3, E2F1, GLIS3, GATA1, DLX3, LHX2, BARX1, HOXC9, FOXK1, RUNX2, RUNX1, SOX4, RREB1, HES6, ASCL1, FOXA3, HOXB2, DLX4, GRHL1, FOXA, HIF, E2F6, FOSL1, JUN, JUNB, FOSB, AP-1, NF-1, RFX6, EL4, TCF3, TCF12, SNAI2, REST, DMRTA2, RFX7, NRF1, ZNF148, ZNF652, PRDM1, HIF1A, TGIF1, STAT2, ESRRA, RELB, HSF1, MAFB, TFAP2C, YBX1, YY1, PITX1, SATB1, ARID3A, POU3F1, SP4, MGA, SALL4, AHR, MLXIP, PRDM4, NFIL3, TFAP2A, ZBTB17, ZFP91, ARID5A, IRF6, ZFX, POU2F1, NKX2-1, NKX2-8, FOXA1, NFKB1, HNF4G, ARID1A, NFATC2, SMAD2, ARID3B, TP53, FOS, FOS-CREB, ELK3, FOXO1::ELK3, TCF7, E2F2, CREB3L1, SHOX2, TCF7L1, HOXA1, MYBL2, NR2C2, MYCN, FOXN1, PITX2, EN2, NFIC, MYC, DLX4, SP3, FOXE1, VENTX, TP53, GLIS3, CUX1, MGA, DLX1, DLX6, GATA1, RUNX2, E2F7, GRHL1, ZBTB7B, HNF1A, FOXA3, NPAS2, TP63, RREB1, SOX4, ZIC2, TCF7, EN1, DMBX1, E2F8, FOSL2, PBX3, NKX3-2, DLX3, HOXB7, TRPS1, SOX11, PAX8, HES6, HOXC10, MNX1, SIX2, ZNF281, ETV4, ZNF384, ASCL1, BARX1, PAX7, LHX2, OTX1, RUNX1, ETV6, FOXK1, HOXB9, E2F4, NR2F6, TWIST1 HOXC9, IRF6, NR2E1, RORB, E2F1, E2F3, TFDP1, FOXJ3, SIX4, MAX::MYC, ONECUT1, or NFκB.

In some embodiments, transcription factors enriched in lung adenocarcinoma (LUAD) can comprise E2F2, CREB3L1, SHOX2, TCF7L1, HOXA1, MYBL2, NR2C2, MYCN, FOXN1, PITX2, EN2, NFIC, MYC, DLX4, SP3, FOXE1, VENTX, TP53, GLIS3, CUX1, MGA, DLX1, DLX6, GATA1, RUNX2, E2F7, GRHL1, ZBTB7B, HNF1A, FOXA3, NPAS2, TP63, RREB1, SOX4, ZIC2, TCF7, EN1, DMBX1, E2F8, FOSL2, PBX3, NKX3-2, DLX3, HOXB7, TRPS1, SOX11, PAX8, HES6, HOXC10, MNX1, SIX2, ZNF281, ETV4, ZNF384, ASCL1, BARX1, PAX7, LHX2, OTX1, RUNX1, ETV6, FOXK1, HOXB9, E2F4, NR2F6, TWIST1, HOXC9, IRF6, NR2E1, RORB, E2F1, E2F3, TFDP1, FOXJ3, SIX4, MAX::MYC, or ONECUT1.

In some embodiments, transcription factors can comprise E2F4, E2F3, E2F1, GLIS3, GATA1, DLX1, DLX3, LHX2, BARX1, PBX3, HOXC9, FOXK1, FOXA3, TRPS1, RUNX2, HOXA1, NFE2L2, TCF3, TCF12, SNAI2, REST, DMRTA2, RFX7, NRF1, ZNF148, ZNF652, PRDM1, HIF1A, TGIF1, STAT2, ESRRA, RELB, HSF1, MAFB, TFAP2C, YBX1, YY1, PITX1, SATB1, ARID3A, USF2, POU3F1, SP4, MGA, SALL4, AHR, MLXIP, MTF1, PRDM4, ZBTB7B, NFIL3, TFAP2A, ZBTB17, ZFP91, BACH1, MLXIP, ARID5A, IRF6, ZFX, POU2F1, NKX2-1, NKX2-8, FOXA1, NFKB1, MGA, HNF4G, ARID1A, NFATC2, POU2F2, SMAD2, PRDM4, MLXIP, or ARID3B. In some embodiments, control TF tiles can comprise TCF7_v2, TCF7L1_v19, TP53_v5, TP53_v22, Control-1-FOSL1_v1, HOXC10_v24, HOXC10_v14, CREB3L1_v6, CREB3L1_v14, Control-Filler_v1, Control-Filler_v2, Control-Filler_v3, Control-Filler_v4, or Control-Filler_v5. In some embodiments, TF tiles can comprise homotypic TF-tiles or heterotypic TF tiles. For examples, TF-tiles comprising mixed binding sequences/sites/motifs from the same TF can be referred to as homotypic TF-tiles. For example, TF-tiles comprising mixed binding sequences/sites/motifs from different TF can be referred to as heterotypic TF-tiles. In some embodiments, SREs can comprise binding sequences, sites, or motifs of TFs of dysregulated genes that are involved in the EGFR, KRAS or p53 pathways in NSCLC.

In some embodiments, a binding site for a transcription factor can comprise a known transcription factor binding site (TFBS) sequence element or DNA binding site sequence element. In some embodiments, a transcription factor can bind to TFBS sequence element or DNA binding site sequence element and can recruit additional transcriptional machinery and co-factors (e.g., RNA polymerase, etc.) to the promoter or the core promoter. In some embodiments, a transcription factor can comprise a transcription co-factor.

In one embodiment, transcription factors that bind to the plurality of transcription binding sites can drive the expression of an ORF operably linked to the promoter in one specific type of cancer cells. In another embodiment, transcription factors that bind to the plurality of transcription binding sites can drive the expression of an ORF operably linked to the promoter in two or more types of cancer cells.

In some embodiments, an SRE can comprise at least about one, at least about two, at least about three, at least about four, at least about five, at least about six, at least about seven, at least about eight, at least about nine, or at least about ten binding sites for one or more transcription factors. In some embodiments, an SRE can comprise at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, or at least about 50 binding sites for one or more transcription factors. In some embodiments, an SRE can comprise at most about 50, at most about 45, at most about 40, at most about 35, at most about 30, at most about 25, at most about 24, at most about 23, at most about 22, at most about 21, at most about 20, at most about 19, at most about 18, at most about 17, at most about 16, at most about 15, at most about 14, at most about 13, at most about 12, at most about 11, at most about 10, at most about 9, at most about 8, at most about 7, at most about 6, or at most about 5 binding sites for one or more transcription factors.

In some embodiments, an SRE can comprise a plurality of binding sites for at least about one, at least about two, at least about three, at least about four, at least about five, at least about six, at least about seven, at least about eight, at least about nine, or at least about ten transcription factors. In some embodiments, an SRE can comprise a plurality of binding sites for at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, or at least about 50 transcription factors. In some embodiments, an SRE can comprise a plurality of binding sites for at most about 50, at most about 45, at most about 40, at most about 35, at most about 30, at most about 25, at most about 24, at most about 23, at most about 22, at most about 21, at most about 20, at most about 19, at most about 18, at most about 17, at most about 16, at most about 15, at most about 14, at most about 13, at most about 12, at most about 11, at most about 10, at most about 9, at most about 8, at most about 7, at most about 6, or at most about 5 transcription factors.

In some embodiments, an SRE can comprise two or more transcription factor binding sites for one transcription factor, wherein each of the two or more transcription factor binding sites can be sequentially arranged or tiled in a sequential manner. For example, an SRE can comprise two or more transcription factor binding site sequences for one transcription factor and each of the two or more transcription factor binding sites can be sequentially arranged or tiled in a sequential manner (e.g., arranged side by side). In some embodiments, an SRE can comprise two or more transcription factor binding sites for one transcription factor, wherein each of two or more transcription factor binding sites can be sequentially arranged or tiled in a sequential manner at 5′ to a core promoter in the recombinant polynucleotide comprising the SRE and the core promoter.

In some embodiments, an SRE can comprise two or more transcription factor binding sites for two or more transcription factors, wherein each of two or more transcription factor binding sites can be non-sequentially arranged or tiled in a non-sequential manner. For example, an SRE can comprise two or more transcription factor binding site sequences for two or more transcription factors and the two or more transcription factor binding site sequences may be (i) the same, (ii) different, or (iii) a combination of (i) and (ii). In this example, the two or more transcription binding sites can comprise (ii) different transcription factor binding site sequences that are non-sequentially arranged or tiled in a non-sequential manner (e.g., shuffled) in the recombinant polynucleotide. In another example, the two or more transcription factor binding sites can comprise (iii) a combination of the same and different transcription factor binding site sequences, wherein all of the two or more transcription factor binding sites are non-sequentially arranged or tiled in a non-sequential manner in the recombinant polynucleotide. In yet another example, the two or more transcription factor binding sites can comprise (iii) a combination of the same and different transcription factor binding site sequences, wherein some of the two or more transcription factor binding sites are sequentially arranged or tiled in a sequential manner and the some of the two or more transcription factor binding sites are non-sequentially arranged or tiled in a non-sequential manner in the recombinant polynucleotide. In some embodiments, an SRE can comprise two or more transcription factor binding sites for two or more transcription factors, wherein each of two or more transcription factor binding sites can be non-sequentially arranged or tiled in a non-sequential manner at 5′ to a core promoter in the recombinant polynucleotide comprising the SRE and the core promoter.

In some embodiments, an SRE comprising a plurality of binding sites for one or more transcription factors can further comprise a spacer element between each of the plurality of binding sites for one or more transcription factors. In some embodiments, a spacer element can comprise a nucleotide sequence of from about 1 to about 10 nucleotides or base pairs. For example, a spacer element can comprise a nucleotide sequence of from about 1 to about 10 nucleotides, from about 2 to about 15 nucleotides, from about 3 to about 20 nucleotides, from about 4 to about 25 nucleotides, from about 4 to about 30 nucleotides, from about 5 to about 35 nucleotides, from about 6 to about 40 nucleotides, from about 7 to about 50 nucleotides, from about 8 to about 55 nucleotides, from about 9 to about 60 nucleotides, from about 10 to about 65 nucleotides, from about 15 to about 70 nucleotides, from about 20 to about 75 nucleotides, from about 25 to about 80 nucleotides, from about 30 to about 85 nucleotides, from about 35 to about 90 nucleotides, from about 40 to about 95 nucleotides, or from about 45 to about 100 nucleotides. In some embodiments, a spacer element can comprise a nucleotide sequence of at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 nucleotides. In some embodiments, a spacer element can comprise a nucleotide sequence of at most about 100, at most about 95, at most about 90, at most about 85, at most about 80, at most about 75, at most about 70, at most about 65, at most about 60, at most about 55, at most about 50, at most about 45, at most about 40, at most about 35, at most about 30, at most about 25, at most about 24, at most about 23, at most about 22, at most about 21, at most about 20, at most about 19, at most about 18, at most about 17, at most about 16, at most about 15, at most about 14, at most about 13, at most about 12, at most about 11, or at most about 10 nucleotides. In some embodiments, a spacer element can comprise a nucleotide sequence of 0, 3, 7, or 10 nucleotides or base pairs.

In some embodiments, an SRS can comprise a plurality of binding sites for one or more transcription factors (TFs), wherein said one or more TFs are expressed at higher levels in cancer cells compared to non-cancer cells. For example, the one or more TFs core promoter may be expressed at a level that is at least 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 110%, 920%, 930%, 940%, 950%, 960%, 970%, 980%, 990%, or at least 1000% higher in cancer cells compared to non-cancer cells.

In some embodiments, an SRS can comprise a plurality of binding sites for one or more transcription factors (TFs), wherein said one or more TFs are more active in cancer cells compared to non-cancer cells. For example, the one or more TFs may be at least 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 110%, 920%, 930%, 940%, 950%, 960%, 970%, 980%, 990%, or at least 1000% more active in cancer cells compared to non-cancer cells. In some embodiments, a phosphorylation assay can be used to measure activation or activity levels of TFs described herein.

### Synthetic Response Elements—Enhancers

In some embodiments, an SRE can comprise a plurality of enhancers. For example, an SRE can comprise a plurality of any known enhancers that can increase the level of transcription of a gene. In some embodiments, an SRE can comprise a plurality of endogenous enhancer sequences. In some embodiments, an SRE can comprise a plurality of enhancers derived from a cancer-responsive gene described herein. In some embodiments, a cancer-responsive gene can comprise a human cancer-responsive gene. In some embodiments, an SRE can comprise at least about one, at least about two, at least about three, at least about four, at least about five, at least about six, at least about seven, at least about eight, at least about nine, or at least about ten enhancers derived from a cancer-responsive gene. In some embodiments, an SRE can comprise at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, or at least about 50 enhancers derived from a cancer-responsive gene. In some embodiments, an SRE can comprise at most about 50, at most about 45, at most about 40, at most about 35, at most about 30, at most about 25, at most about 24, at most about 23, at most about 22, at most about 21, at most about 20, at most about 19, at most about 18, at most about 17, at most about 16, at most about 15, at most about 14, at most about 13, at most about 12, at most about 11, at most about 10, at most about 9, at most about 8, at most about 7, at most about 6, or at most about 5 enhancers derived from a cancer-responsive gene.

In some embodiments, an SRE can comprise a plurality of enhancers derived from two or more cancer-responsive genes described herein. In some embodiments, a cancer-responsive gene can refer to a gene specifically or preferentially expressed in cancer cells or cancer tissues compared to non-cancer cells or non-cancer tissues. In some embodiments, a cancer-responsive gene can comprise a human cancer-responsive gene. In some embodiments, an SRE can comprise a plurality of enhancers derived from at least about two, at least about three, at least about four, at least about five, at least about six, at least about seven, at least about eight, at least about nine, or at least about ten cancer-responsive genes. In some embodiments, an SRE can comprise a plurality of enhancers derived from at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 cancer-responsive genes. In some embodiments, an SRE can comprise a plurality of enhancers derived from at most about 100, at most about 95, at most about 90, at most about 85, at most about 80, at most about 75, at most about 70, at most about 65, at most about 60, at most about 55, at most about 50, at most about 45, at most about 40, at most about 35, at most about 30, at most about 25, at most about 24, at most about 23, at most about 22, at most about 21, at most about 20, at most about 19, at most about 18, at most about 17, at most about 16, at most about 15, at most about 14, at most about 13, at most about 12, at most about 11, at most about 10, at most about 9, at most about 8, at most about 7, at most about 6, or at most about 5 cancer-responsive genes.

In some embodiments, a plurality of enhancers described herein can comprise a transcription regulatory element (TRE). A TRE can refer to a region of DNA that can regulate transcription of a gene. In some embodiments, a TRE can increase the transcription of a gene. In some embodiments, a TRE can decrease the transcription of a gene. In some embodiments, a TRE can comprise a transcription binding site. In some embodiments, a plurality of enhancers can comprise a transcription regulatory element that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence homology to an enhancer consensus sequence of two or more homologous cancer-responsive genes. In some embodiments, a plurality of enhancers can comprise a transcription regulatory element that has 90% sequence homology to an enhancer consensus sequence of two or more homologous cancer-responsive genes.

In some embodiments, a plurality of enhancers can comprise an enhancer consensus sequence of two or more homologous cancer-responsive genes. In some embodiments, an enhancer consensus sequence of two or more homologous cancer-responsive genes can comprise a consensus sequence of an enhancer sequence derived from two or more cancer-responsive genes that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity between the two or more cancer-responsive genes. In some embodiments, an enhancer consensus sequence of two or more homologous cancer-responsive genes can comprise a consensus sequence of an enhancer sequence derived from two or more cancer-responsive genes that has at least 90% sequence identity between the two or more cancer-responsive genes.

In some embodiments, an SRE can comprise a plurality of enhancers comprising at least two enhancer sequences, wherein each of the at least two enhancer sequences can comprise (i) the same enhancer sequences, (ii) different enhancer sequences, or (iii) a combination of (i) and (ii). In some embodiments, each of the at least two enhancer sequences can be sequentially arranged or tiled in a sequential manner in a recombinant polynucleotide. In some embodiments, each of the at least two enhancer sequences can be sequentially arranged or tiled in a sequential manner at 5′ to a core promoter in the recombinant polynucleotide comprising the core promoter and an SRE comprising the plurality of enhancers. In some embodiments, each of said at least two enhancer sequences can be sequentially arranged or tiled in a sequential manner at 5′ to a core promoter and/or at 3′ to a plurality of binding sites for one or more TFs, if present, in the recombinant polynucleotide comprising the core promoter, an SRE comprising the plurality of enhancers, and/or the plurality of transcription factor binding sites.

In some embodiments, an SRE can comprise a plurality of enhancers comprising at least two enhancer sequences, wherein each of the at least two enhancer sequences can comprise (ii) different enhancer sequences. In this embodiment, each of said plurality of enhancers comprising different enhancer sequences can be non-sequentially arranged or tiled in a non-sequential manner. In some embodiments, each of said plurality of enhancers comprising different enhancer sequences can be non-sequentially arranged or tiled in a non-sequential manner at 5′ to a core promoter in the recombinant polynucleotide comprising the core promoter and an SRE comprising the plurality of enhancers. In some embodiments, each of said plurality of enhancers comprising different enhancer sequences can be non-sequentially arranged or tiled in a non-sequential manner at 5′ to a core promoter and/or at 3′ to a plurality of binding sites for one or more TFs, if present, in the recombinant polynucleotide comprising the core promoter, an SRE comprising the plurality of enhancers, and/or the plurality of transcription factor binding sites.

In some embodiments, an SRE can comprise a plurality of enhancers comprising at least two enhancer sequences, wherein each of the at least two enhancer sequences can comprise (iii) a combination of the same and different enhancer sequences. In this embodiment, each of said plurality of enhancers comprising a combination of the same and different enhancer sequences can be non-sequentially arranged or tiled in a non-sequential manner. In some embodiments, each of said plurality of enhancers comprising a combination of the same and different enhancer sequences can be non-sequentially arranged or tiled in a non-sequential manner at 5′ to a core promoter in the recombinant polynucleotide comprising the core promoter and an SRE comprising the plurality of enhancers. In some embodiments, each of said plurality of enhancers comprising a combination of the same and different enhancer sequences can be non-sequentially arranged or tiled in a non-sequential manner at 5′ to a core promoter and/or at 3′ to a plurality of binding sites for one or more TFs, if present, in the recombinant polynucleotide comprising the core promoter, an SRE comprising the plurality of enhancers, and/or the plurality of transcription factor binding sites.

In some embodiments, a plurality of enhancers described herein can comprise a sequence capable of binding to a transcription associated protein. A transcription associated protein as described herein can comprise any protein that is involved in transcription of a DNA sequence to an RNA sequence. In some embodiments, a transcription associated protein can bind to an enhancer sequence. In some embodiments, an assay can be used to determine if a transcription associated protein can bind to a sequence comprised in a plurality of enhancers. For example, chromatin immunoprecipitation (ChIP) assay, an in vitro transfection reporter assay, or any other suitable assays or methods can be used to determine if a transcription associated protein can bind to a sequence comprised in a plurality of enhancers. In some embodiments, a plurality of enhancers described herein can comprise a sequence capable of binding to a transcription associated protein determined by chromatin immunoprecipitation (ChIP) or an in vitro transfection reporter assay.

In some embodiments, a plurality of enhancers can comprise a CpG island. For example, at least one enhancer of the plurality of enhancers can comprise a CpG island. In some embodiments, a plurality of enhancers may not comprise a CpG island. For example, at least one enhancer of the plurality of enhancers may not comprise a CpG island.

In some embodiments, an SRS can comprise a core promoter and a plurality of binding sites for one or more transcription factors derived from two or more cancer-responsive genes, wherein the core promoter and the plurality of binding sites for one or more transcription factors are not derived from the same cancer-responsive gene. In some embodiments, an SRS can comprise a core promoter and a plurality of enhancers derived from two or more cancer-responsive genes, wherein the core promoter and the plurality of enhancers are not derived from the same cancer-responsive gene. In some embodiments, an SRS can comprise a core promoter, a plurality of binding sites for one or more transcription factors, and a plurality of enhancer derived from two or more cancer-responsive genes, wherein the core promoter, the plurality of binding sites for one or more transcription factors, and the plurality of enhancer are not derived from the same cancer-responsive gene. In some embodiments, a cancer-responsive gene can comprise a human cancer-responsive gene.

In some embodiments, a plurality of enhancers can comprise an enhancer sequence that can bind to SP1, ETS, CEBP, NF-KB, EBS, C/EBP, ARE, DRE, NFκB, GC-box, UN5CL, BOP1, RTN4RL2, ARNTL2, AGR2, LHX2, TRNP1, MU5AC, or DOK4. In some embodiments, a plurality of enhancers can comprise at least two, at least about three, at least about four, at least about five, at least about six, at least about seven, at least about eight, at least about nine, or at least about ten enhancer sequences. In some embodiments, a plurality of enhancers can comprise at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 enhancer sequences. In some embodiments, a plurality of enhancers can comprise at least two SP1, ETS, CEBP, NF-KB, EBS, C/EBP, ARE, DRE, NFκB, GC-box, UN5CL, BOP1, RTN4RL2, ARNTL2, AGR2, LHX2, TRNP1, MU5AC, or DOK4 enhancer sequences.

In some embodiments, core promoter, plurality of binding sites for one or more transcription factors, or plurality of enhancers derived from two or more cancer-responsive genes can comprise a sequence listed in Table 1A, Table 1B, or Table 1C. In some embodiments, an SRS described herein can comprise a sequence listed in Table 1A, Table 1B, or Table 1C.

In some embodiments, an SRS can comprise a sequence comprising a human alpha-fetoprotein (AFP) promoter sequence comprising a plurality of HNF-1A transcription binding sites. AFP level is elevated in liver cancer including, but not limited to, hepatic carcinomas. In some embodiments, an HNF-1A transcription binding site can comprise a sequence of 5′-GTTAATTATTAAC-3′ (SEQ ID NO: 128).

### Cancer Cells or Cell Lines

Described herein is a method of selectively expressing a protein in cancer or tumor cells. In some embodiments, the method can comprise contacting cancer or tumor cells with a recombinant polynucleotide comprising any SRS described herein that comprises a promoter or a core promoter, one or more SREs, and an open reading frame (ORF) encoding a protein. In some embodiments, the ORF can be operatively linked to the SRS or the promoter (or the core promoter) in the SRS. In some embodiments, cancer or tumor cells described herein can comprise malignant cancer cells. Examples of cancer or tumor cells include, but are not limited to, colorectal cancer (CRC) cells, hepatocellular carcinoma cells, breast cancer cells, or lung cancer cells. In some embodiments, cancer or tumor cells can comprise cancer or tumor cells associated with colorectal cancer (CRC), hepatocellular carcinoma, lung cancer, liver cancer, breast cancer, prostate cancer, cervix cancer, uterus cancer, pancreas cancer, kidney cancer, stomach cancer, bladder cancer, ovary cancer, brain cancer, head and neck cancer, eye cancer, mouth cancer, throat cancer, esophagus cancer, chest cancer, bone cancer, rectum or other gastrointestinal tract organ cancer, spleen cancer, skeletal muscle cancer, subcutaneous tissue cancer, testicles or other reproductive organ cancer, skin cancer, thyroid cancer, blood cancer, or lymph nodes cancer. In some embodiments, adenocarcinoma (LUAD) cells can comprise LXFA586, LXFA629, LXFA2184, or A549. In some embodiments, large cell carcinoma cells can comprise H1299, LXFL430, LXFL1121, or LXFL529. In some embodiments, squamous cell carcinoma (LUSC) cells can comprise LK2, H520, H1703, SK-MES-1, or Calu-1. In some embodiments, hepatocellular carcinoma (HCC) cells can comprise HUH7.

In some embodiments, promoters active in LXFA586 cell lines can comprise promoters of TP53, HES6, FOS, FOS-CREB, FOXO1::ELK3, or MTF1. In some embodiments, promoters active in LXFA629 cell lines can comprise promoters of FOS, CREB3L1, or HES6. In some embodiments, promoters active in LXFA2184 cell lines can comprise promoters of FOS or MNX. In some embodiments, promoters active in H1299 cell lines can comprise promoters of FOS, CREB3L1, HES6, FOS-CREB, NFE2L2, FOXO1::ELK3, or XBP1. In some embodiments, promoters active in LXFL430 cell lines can comprise promoters of TCF7, ETV4, HOXC10, FOS-CREB, FOXO1::ELK3, or XBP1. In some embodiments, promoters active in LXFL1121 cell lines can comprise promoters of FOS, CREB3L1, or ETV4. In some embodiments, promoters active in LXFL529 cell lines can comprise promoters of FOS.

In some embodiments, expression of the protein encoded by the ORF may be increased in cancer cells compared to non-cancer cells. In some embodiments, expression of the protein encoded by the ORF may be increased when the recombinant polynucleotide comprising the SRS and the ORF is introduced to cancer cells compared to non-cancer cells. For example, expression of the protein encoded by the ORF may be increased at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, or at least about 250% in cancer cells compared to non-cancer cells. In some embodiments, the ORF can comprise a sequence encoding a therapeutic protein, marker protein (e.g., for diagnostic imaging, etc.), or a reporter protein (e.g., luciferase). In some embodiments, the ORF can comprise a sequence encoding a recombinant, synthetic, or engineered protein.

In some embodiments, expression of the protein encoded by the ORF may be increased in a first plurality of cancer cells when said recombinant polynucleotide is introduced to the first plurality of cancer cells compared to a second plurality of cancer cells, wherein the first plurality of cancer cells and the second plurality of cancer cells are different types of cancer cells. In some embodiments, expression of the protein encoded by the ORF may be increased in a first plurality of cancer cells when the recombinant polynucleotide comprising the SRS and the ORF is introduced to the first plurality of cancer cells compared to a second plurality of cancer cells, wherein the first plurality of cancer cells and the second plurality of cancer cells are different types of cancer cells. For example, expression of the protein encoded by the ORF operatively linked to a first type of SRS in the recombinant polynucleotide may be increased in cells of one type of cancer in which the first type of SRS can drive expression of the ORF compared to in cells of another type of cancer in which the first type of SRS cannot drive expression of the ORF. For example, expression of the protein encoded by the ORF operatively linked to an SRS that is specific for lung cancer may be increased in lung cancer cells compared to in liver cancer cells.

In some embodiments, expression of the protein encoded by the ORF may be increased in a first plurality of cancer cells comprising two or more types of cancer cells when the recombinant polynucleotide comprising the SRS and the ORF is introduced to the first plurality of cancer cells compared to a second plurality of cancer cells. For example, expression of the protein encoded by the ORF operatively linked to a first type of SRS in the recombinant polynucleotide may be increased in cells of two or more types of cancer in which the first type of SRS can drive expression of the ORF compared to in cells of another type of cancer in which the first type of SRS cannot drive expression of the ORF. For example, expression of the protein encoded by the ORF operatively linked to an SRS that is specific for lung and liver cancer may be increased in lung cancer cells and liver cancer cells compared to in non-lung cancer cells and non-liver cancer cells (e.g., breast cancer cells, etc.). In some embodiments, the first plurality of cancer cells comprising two or more types of cancer cells can comprise cells associated with two or more cancers comprising colorectal cancer, hepatocellular carcinoma, lung cancer, liver cancer, breast cancer, prostate cancer, cervix cancer, uterus cancer, pancreas cancer, kidney cancer, stomach cancer, bladder cancer, ovary cancer, brain cancer, head and neck cancer, eye cancer, mouth cancer, throat cancer, esophagus cancer, chest cancer, bone cancer, rectum or other gastrointestinal tract organ cancer, spleen cancer, skeletal muscle cancer, subcutaneous tissue cancer, testicles or other reproductive organ cancer, skin cancer, thyroid cancer, blood cancer, or lymph nodes cancer.

### Therapeutic or Diagnostic Applications

Provided herein are recombinant polynucleotides (or any vector, pharmaceutical composition, or lipid nanoparticle comprising any recombinant polynucleotides described herein) useful for the diagnosis or the treatment of a disease or condition. In some aspects, recombinant polynucleotides described herein (or any vector, pharmaceutical composition, or lipid nanoparticle comprising any recombinant polynucleotides described herein) are present or administered in an amount for sufficient expression of a protein (e.g., a reporter protein or a biomarker) useful for a diagnosis of a disease or condition. In some embodiments, the disease or condition comprise a cancer. In some aspects, provided herein is a method of selectively expressing a reporter protein or a biomarker in a cancer or tumor cell. In some aspects, the method comprises contacting a tumor cell with any of recombinant polynucleotides described herein, any of vectors comprising recombinant polynucleotide described herein, any of pharmaceutical composition comprising recombinant polynucleotide described herein, or any of lipid nanoparticle (LNP) comprising the recombinant polynucleotide, the vector, or the pharmaceutical composition described herein, wherein recombinant polynucleotides can comprise an open reading frame (ORF) encoding the reporter protein or the biomarker operatively linked to a synthetic promoter described herein (e.g., a synthetic promoter that can drive expression of the ORF preferentially or specifically in cancer cells).

In some aspects, provided herein is a method for diagnosing a disease or a condition. In some embodiments, the method can comprise administering to any of recombinant polynucleotide described herein, a vector comprising the recombinant polynucleotide described herein, the pharmaceutical composition comprising the recombinant polynucleotide described herein, or a lipid nanoparticle (LNP) comprising the recombinant polynucleotide, the vector, or the pharmaceutical composition described herein to a subject. In some embodiments, the recombinant polynucleotide can further comprise an open reading frame (ORF) encoding a reporter protein or a biomarker, wherein the ORF is operatively linked to a synthetic promoter in the recombinant polynucleotide that can drive expression of the ORF selectively, preferentially, or specifically in diseased cells compared to non-disease cells. In some embodiments, the method can further comprise detecting the reporter protein or a biomarker of which expression can be induced by a synthetic promoter in the recombinant polynucleotide described herein selectively, preferentially, or specifically in diseased cells compared to non-disease cells. In some embodiments, a relative ratio of the reporter protein or the biomarker expressed in the diseased cells over the non-diseased cells can be greater than 1.0. For example, a relative ratio of the reporter protein or the biomarker expressed in the diseased cells over the non-diseased cells can be greater than about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 20.0, 25.0, 30.0, 35.0, 40.0, 45.0, 50.0, 55.0, 60.0, 65.0, 70.0, 75.0, 80.0, 85.0, 90.0, 95.0, or about 100.0. In some embodiments, the disease or condition can comprise a cancer.

In some aspects, recombinant polynucleotides (or any vector, pharmaceutical composition, or lipid nanoparticle comprising any recombinant polynucleotides described herein) are present or administered in an amount sufficient to treat or prevent a disease or condition. In some aspects, provided herein, is a method of treating a disease or condition comprising administering to a subject in need thereof the recombinant polynucleotide described herein, a vector comprising the recombinant polynucleotide described herein, a pharmaceutical composition comprising the recombinant polynucleotide described herein, or a lipid nanoparticle (LNP) comprising the vector, the pharmaceutical composition or the recombinant polynucleotide described herein. In some aspects, provided herein, is recombinant polynucleotide described herein, a vector comprising the recombinant polynucleotide described herein, the pharmaceutical composition comprising the recombinant polynucleotide described herein, or a lipid nanoparticle (LNP) comprising the recombinant polynucleotide, the vector, or the pharmaceutical composition described herein for use in a method of treating a disease or a condition in a subject in need thereof. In some aspects, provided herein, is the use of recombinant polynucleotide described herein, a vector comprising the recombinant polynucleotide described herein, the pharmaceutical composition comprising the recombinant polynucleotide described herein, or a lipid nanoparticle (LNP) comprising the recombinant polynucleotide, the vector, or the pharmaceutical composition described herein for the manufacture of a medicament for treating a disease or a condition in a subject in need thereof.

In some aspects, provided herein is a method for treating a subject having or suspected of having a disease or a condition. In some embodiments, the method can comprise administering any of recombinant polynucleotide described herein, a vector comprising the recombinant polynucleotide described herein, the pharmaceutical composition comprising the recombinant polynucleotide described herein, or a lipid nanoparticle (LNP) comprising the recombinant polynucleotide, the vector, or the pharmaceutical composition described herein to a subject. In some embodiments, the recombinant polynucleotide can further comprise an open reading frame (ORF) encoding a therapeutic protein, wherein the ORF is operatively linked to a synthetic promoter in the recombinant polynucleotide that can drive expression of the ORF selectively, preferentially, or specifically in diseased cells compared to non-disease cells. In some embodiments, a relative ratio of the therapeutic protein expressed in the diseased cells over the non-diseased cells can be greater than 1.0. For example, a relative ratio of the therapeutic protein expressed in the diseased cells over the non-diseased cells can be greater than about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, or about 15.0.

In some embodiments, the disease or disorder can comprise a cancer. Examples of cancer can include, but are not limited to, colorectal cancer (CRC), hepatocellular carcinoma, breast cancer, lung cancer, liver cancer, prostate cancer, cervix cancer, uterus cancer, pancreas cancer, kidney cancer, stomach cancer, bladder cancer, ovary cancer, brain cancer, head and neck cancer, eye cancer, mouth cancer, throat cancer, esophagus cancer, chest cancer, bone cancer, rectum or other gastrointestinal tract organ cancer, spleen cancer, skeletal muscle cancer, subcutaneous tissue cancer, testicles or other reproductive organ cancer, skin cancer, thyroid cancer, blood cancer, or lymph nodes cancer.

Also provided herein are pharmaceutical compositions comprising any recombinant polynucleotide described herein or any vector comprising the recombinant polynucleotide described herein and a pharmaceutically acceptable excipient, carrier, or diluent. A pharmaceutical composition can denote a mixture or solution comprising a therapeutically effective amount of an active pharmaceutical ingredient together with one or more pharmaceutically acceptable excipients to be administered to a subject in need thereof. The term “pharmaceutically acceptable” can denote an attribute of a material which is useful in preparing a pharmaceutical composition that is generally safe, non-toxic, and neither biologically nor otherwise undesirable and is acceptable for veterinary as well as human pharmaceutical use. The term “Pharmaceutically acceptable” can refer to a material, such as a excipient, carrier, or diluent, which does not abrogate the biological activity or properties of the recombinant polynucleotide or the compound, and is relatively nontoxic, i.e., the material may be administered to an individual without causing undesirable biological effects or interacting in a deleterious manner with any of the components of the composition in which it is contained. A pharmaceutically acceptable excipient can denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents, excipients, preservatives, or lubricants used in formulating pharmaceutical products. Pharmaceutical compositions can facilitate administration of a recombinant polynucleotide, a vector comprising recombinant polynucleotide, or a compound to an organism and can be formulated in a conventional manner using one or more pharmaceutically acceptable inactive ingredients that facilitate processing of the active compounds into preparations that can be used pharmaceutically. A proper formulation is dependent upon the route of administration chosen and a summary of pharmaceutical compositions can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference. In some embodiments, pharmaceutical compositions can be formulated by dissolving active substances (e.g., recombinant polynucleotides or vectors comprising the recombinant polynucleotides described herein) in aqueous solution for administration into a cell, a tissue or a subject (e.g., a disease cell, disease tissue, or a subject in need thereof). In some embodiments, pharmaceutical compositions can be formulated by dissolving active substances (e.g., recombinant polynucleotides or vectors comprising the recombinant polynucleotides described herein) in aqueous solution for administration into a cell, a tissue or a subject (e.g., a disease cell, disease tissue, or a subject in need thereof).

Also provided herein are methods of treating a disease or condition in a subject in need thereof, comprising administering to the subject a therapeutically effective amount of any recombinant polynucleotide described herein, any vector comprising recombinant polynucleotide described herein, or pharmaceutical compositions described herein. The terms “effective amount” or “therapeutically effective amount,” as used herein, can refer to a sufficient amount of an agent, a compound, any recombinant polynucleotide described herein, any vector comprising recombinant polynucleotide described herein, or pharmaceutical compositions described herein being administered which will relieve to some extent one or more of the symptoms of the disease or the condition being treated; for example a reduction and/or alleviation of one or more signs, symptoms, or causes of a disease, or any other desired alteration of a biological system. For example, an “effective amount” for therapeutic uses can be an amount of an agent that provides a clinically significant decrease in one or more disease symptoms. An appropriate “effective” amount may be determined using techniques, such as a dose escalation study, in individual cases. In some embodiments, an “effective amount” can comprise an amount for sufficient expression of a protein (e.g., a reporter protein or a biomarker) useful for diagnosing a disease or condition in a subject.

The terms “treat,” “treating” or “treatment,” as used herein, can include alleviating, abating or ameliorating at least one symptom of a disease or a condition, preventing additional symptoms, inhibiting the disease or the condition, e.g., arresting the development of the disease or the condition, relieving the disease or the condition, causing regression of the disease or the condition, relieving a condition caused by the disease or the condition, or stopping the symptoms of the disease or the condition either prophylactically and/or therapeutically. In some embodiments, treating a disease or condition comprises reducing the size of disease tissues or diseased cells. In some embodiments, treating a disease or a condition in a subject comprises increasing the survival of a subject. In some embodiments, treating a disease or condition comprises reducing or ameliorating the severity of a disease, delaying onset of a disease, inhibiting the progression of a disease, reducing hospitalization of or hospitalization length for a subject, improving the quality of life of a subject, reducing the number of symptoms associated with a disease, reducing or ameliorating the severity of a symptom associated with a disease, reducing the duration of a symptom associated with a disease, preventing the recurrence of a symptom associated with a disease, inhibiting the development or onset of a symptom of a disease, or inhibiting of the progression of a symptom associated with a disease. In some embodiments, treating a cancer comprises reducing the size of tumor or increasing survival of a patient with a cancer.

In some cases, a subject can encompass mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In some cases, the mammal is a human. In some cases, the subject may be an animal. In some cases, an animal may comprise human beings and non-human animals. In one embodiment, a non-human animal may be a mammal, for example a rodent such as rat or a mouse. In another embodiment, a non-human animal may be a mouse. In some instances, the subject is a mammal. In some instances, the subject is a human. In some instances, the subject is an adult, a child, or an infant. In some instances, the subject is a companion animal. In some instances, the subject is a feline, a canine, or a rodent. In some instances, the subject is a dog or a cat.

Recombinant polynucleotides, vectors, or pharmaceutical compositions described herein can be administered to a subject using any suitable methods known in the art. Suitable formulations for use in the present invention and methods of delivery are generally well known in the art. For example, compositions described herein can be administered to the subject in a variety of ways, including parenterally, intravenously, intradermally, intramuscularly, colonically, rectally, or intraperitoneally. In some embodiments, compositions described herein is administered by intraperitoneal injection, intramuscular injection, subcutaneous injection, or intravenous injection of the subject. In some embodiments, compositions described herein can be administered parenterally, intravenously, intramuscularly or orally. In some embodiments, compositions described herein can be administered via injection into disease tissues or cells.

In some embodiments, compositions or pharmaceutical compositions comprising any recombinant polynucleotide described herein can be delivered to a cell via direct DNA transfer (Wolff et al. (1990) Science 247, 1465-1468). In some embodiments, recombinant polynucleotides can be delivered to cells following mild mechanical disruption of the cell membrane, temporarily permeabilizing the cells. Such a mild mechanical disruption of the membrane can be accomplished by gently forcing cells through a small aperture (Sharei et al. PLOS ONE (2015) 10 (4), e0118803). In another embodiment, compositions or pharmaceutical compositions comprising any recombinant polynucleotide described herein can be delivered to via liposome or lipid nanoparticle (LNP) (e.g., Gao & Huang (1991) Biochem. Ciophys. Res. Comm. 179, 280-285, Crystal (1995) Nature Med. 1, 15-17, Caplen et al. (1995) Nature Med. 3, 39-46). A liposome or LNP can encompass a variety of single and multilamellar lipid vehicles formed by the generation of enclosed lipid bilayers or aggregates. Recombinant polynucleotides can be encapsulated in the aqueous interior of a liposome or LNP, interspersed within the lipid bilayer of a liposome, attached to a liposome via a linking molecule that is associated with both the liposome and the oligonucleotide, entrapped in a liposome, or complexed with a liposome.

In some aspects, provided herein is a method comprising: (a) administering to a subject any of the pharmaceutical composition described herein; or a composition any of the recombinant polynucleotide described herein, any of the vector described herein, or any of the LNP described herein; wherein the recombinant polynucleotide further comprises an open reading frame (ORF) encoding a reporter protein, wherein said ORF is operatively linked to a synthetic promoter in said recombinant polynucleotide, and (b) localizing a tumor or an absence thereof in a body of said subject via expression of said reporter protein using an imaging technique performed on said body of said subject. In some embodiments, the imaging technique comprises photoacoustic imaging, Magnetic resonance imaging (MRI) imaging, positron emission tomography (PET) imaging, or single-photon emission computed tomography (SPECT) imaging.

### Embodiments

In some aspects, provided herein is a recombinant polynucleotide comprising: (a) a core promoter comprising a transcription start site (TSS), wherein the core promoter is derived from one or more cancer-responsive genes that are either expressed at a higher level or are more active in cancer cells compared to non-cancer cells and operably linked to an open reading frame (ORF) and (b) a plurality of binding sites for one or more transcription factors (TFs), wherein said one or more TFs are expressed at higher levels or more active in cancer cells compared to non-cancer cells. In some embodiments, the recombinant polynucleotide further comprises a plurality of enhancers.

In some aspects, provided herein is a recombinant polynucleotide comprising: (a) a core promoter comprising a transcription start site (TSS) and two or more promoter elements derived from two or more cancer-responsive genes that are either expressed at a higher level or are more active in cancer cells compared to non-cancer cells and operably linked to an open reading frame (ORF) and (b) a plurality of binding sites for one or more transcription factors (TFs), wherein said one or more TFs are expressed at higher levels or more active in cancer cells compared to non-cancer cells. In some embodiments, the recombinant polynucleotide further comprises a plurality of enhancers.

In some embodiments, said plurality of enhancers are derived from one or more cancer-responsive genes that are either expressed at a higher level or are more active in cancer cells compared to non-cancer cells. In some embodiments, said plurality of enhancers are derived from two or more cancer-responsive genes that are either expressed at a higher level or are more active in cancer cells compared to non-cancer cells, wherein one of said plurality of enhancers comprises: (i) a transcription regulatory element with at least 90% sequence homology to an enhancer consensus sequence of two or more homologous cancer-responsive genes, and/or (ii) a sequence capable of binding a transcription associated protein as determined by chromatin immunoprecipitation (ChIP) or an in vitro transfection reporter assay.

In some aspects, provided herein is a recombinant polynucleotide comprising: (a) a core promoter comprising a transcription start site (TSS), wherein the core promoter is derived from one or more cancer-responsive genes that are either expressed at a higher level or are more active in cancer cells compared to non-cancer cells and operably linked to an open reading frame (ORF) and (b) a plurality of enhancers. In some embodiments, said plurality of enhancers are derived from one or more cancer-responsive genes that are either expressed at a higher level or are more active in cancer cells compared to non-cancer cells. In some embodiments, said plurality of enhancers are derived from two or more cancer-responsive genes that are either expressed at a higher level or are more active in cancer cells compared to non-cancer cells, wherein one of said plurality of enhancers comprises: (i) a transcription regulatory element with at least 90% sequence homology to an enhancer consensus sequence of two or more homologous cancer-responsive genes, and/or (ii) a sequence capable of binding a transcription associated protein as determined by chromatin immunoprecipitation (ChIP) or an in vitro transfection reporter assay.

In some aspects, provided herein, is a recombinant polynucleotide comprising: (a) a core promoter comprising a transcription start site (TSS), wherein the core promoter is derived from one or more cancer-responsive genes that are either expressed at a higher level or are more active in cancer cells compared to non-cancer cells and operably linked to an open reading frame (ORF), (b) a plurality of binding sites for one or more transcription factors (TFs), wherein said one or more TFs are expressed at higher levels or more active in cancer cells compared to non-cancer cells, and (c) a plurality of enhancers. In some embodiments, said plurality of enhancers are derived from one or more cancer-responsive genes that are either expressed at a higher level or are more active in cancer cells compared to non-cancer cells. In some embodiments, said plurality of enhancers are derived from two or more cancer-responsive genes that are either expressed at a higher level or are more active in cancer cells compared to non-cancer cells, wherein one of said plurality of enhancers comprises: (i) a transcription regulatory element with at least 90% sequence homology to an enhancer consensus sequence of two or more homologous cancer-responsive genes, and/or (ii) a sequence capable of binding a transcription associated protein as determined by chromatin immunoprecipitation (ChIP) or an in vitro transfection reporter assay.

In some embodiments, said core promoter further comprises two or more promoter elements derived from two or more cancer-responsive genes that are either expressed at a higher level or are more active in cancer cells compared to non-cancer cells and operably linked to an open reading frame (ORF). In some embodiments, said one or more cancer-responsive genes are derived from a human subject. In some embodiments, (a) said core promoter, and (b) said plurality of binding sites for one or more TFs or said plurality of enhancers derived from one or more cancer-responsive genes are not derived from a same cancer-responsive gene. In some embodiments, said enhancer consensus sequence of two or more homologous cancer-responsive genes is a consensus sequence of an enhancer sequence derived from two or more cancer-responsive genes that has at least 90% sequence identity between two or more human cancer-responsive genes.

In some embodiments, the recombinant polynucleotide comprises (a) a plurality of binding sites for one or more transcription factors (TFs), wherein one or more TFs are expressed in higher levels or more active in cancer cells compared to non-cancer cells and (b) a plurality of enhancers derived from two or more cancer-responsive genes, wherein each of said plurality of enhancers comprising: (i) a transcription regulatory element with at least 90% sequence homology to an enhancer consensus sequence of two or more homologous cancer-responsive genes, and/or (ii) a sequence capable of binding a transcription associated protein as determined by chromatin immunoprecipitation (ChIP) or an in vitro transfection reporter assay.

In some embodiments, at least one of the plurality of enhancers comprises a CpG island. In some embodiments, at least one of the plurality of enhancers does not comprise a CpG island. In some embodiments, said higher levels of TF expression in cancer cells compared to non-cancer cells is determined by chromatin immunoprecipitation (ChIP).

In some embodiments, the recombinant polynucleotide further comprises an open reading frame (ORF), wherein said core promoter is operably linked to said ORF. In some embodiments, said plurality of binding sites for one or more TFs are 5′ to said core promoter. In some embodiments, said plurality of enhancers are 5′ to said core promoter and 3′ to said plurality of binding sites for one or more TFs, if present. In some embodiments, said plurality of binding sites for one or more TFs comprises two or more binding sites for one TF, wherein each of the plurality of binding sites for one or more TFs is sequentially arranged at 5′ to said core promoter in the recombinant polynucleotide. In some embodiments, said plurality of binding sites for one or more TFs comprises two or more binding sites for two or more TFs, wherein each of the plurality of binding sites for one or more TFs is non-sequentially arranged at 5′ to said core promoter in the recombinant polynucleotide.

In some embodiments, said plurality of binding sites for one or more TFs comprise a plurality of TRPS1, MNX1, TWIST1, ETV4, FOSL2, NFIC, EN2, TFDP1, PITX2, TCF7L1, VENTX, HOXB9, DLX1, MYCN, SIX4, TP63, SOX11, E2F8, TFDP1, SURV, TOXE1, EN1, ZBTB7B, SP3, SIX2, XBP1, HIF-1A, CREB3L1, HSF-1, MTF1, NFE2L2, USF2, TP73, USF2, POU2F2, HOXA1, FOXO1, TFAP4, BACH1, E2F4, HOXC10, KLF11, FOXM1, E2F2, RUNX1, SOX4, RREB1, ETV4, HES6, ASCL1, TWIST1, FOXA3, PITX2, HOXB2, EN2, DLX4, GRHL1, FOXA, HIF, E2F6, FOSL1, NF-1, RFX6, EL4, or NFκB TF binding sites.

In some embodiments, the recombinant polynucleotide further comprises a spacer element comprising 1-10 nucleotides between each of plurality of binding sites for one or more TFs. In some embodiments, said one or more cancer-responsive genes from which said core promoter is derived comprises TCF7, MNX1, HOXC10, TP53, CEACAM5, CEP55, FAM111B, CST1, BIRC5, FOS, TWIST1, E2F2, KIF20A, or ETV4. In some embodiments, said one or more cancer-responsive genes from which said core promoter is derived comprise two or more of TCF7, MNX1, HOXC10, TP53, CEACAM5, CEP55, FAM111B, CST1, BIRC5, FOS, TWIST1, E2F2, KIF20A, or ETV4. In some embodiments, said one or more cancer-responsive genes from which said core promoter is derived comprise TCF7 and HOXC10. In some embodiments, said one or more cancer-responsive genes from which said core promoter is derived comprise TP53 and CEP55. In some embodiments, said one or more cancer-responsive genes from which said core promoter is derived comprise FAM111B and KIF20A. In some embodiments, said one or more cancer-responsive genes from which said core promoter is derived comprise BIRC5 and E2F2. In some embodiments, said one or more cancer-responsive genes from which said core promoter is derived comprise CEACAM5 and TWIST1. In some embodiments, said core promoter comprises a region from about −300 bp to +100 bp relative to said TSS.

In some embodiments, said plurality of enhancers comprises at least two enhancer sequences, wherein each of said at least two enhancer sequences comprises (i) the same enhancer sequences, (ii) different enhancer sequences, or (iii) a combination thereof. In some embodiments, each of said at least two enhancer sequences is sequentially arranged at 5′ to said core promoter in the recombinant polynucleotide. In some embodiments, each of said at least two enhancer sequences is sequentially arranged at 5′ to said core promoter and at 3′ to said plurality of binding sites for one or more TFs, if present, in the recombinant polynucleotide. In some embodiments, each of said at least two enhancer sequences comprises (ii), wherein each of said plurality of enhancers comprising different enhancer sequences is non-sequentially arranged at 5′ to said core promoter in the recombinant polynucleotide. In some embodiments, each of said at least two enhancer sequences comprises (ii), wherein each of said plurality of enhancers is non-sequentially arranged at 5′ to said core promoter and at 3′ to said plurality of binding sites of one or more TF binding sites, if present, in the recombinant polynucleotide. In some embodiments, each of said at least two enhancer sequences comprises (iii), wherein each of said plurality of enhancers comprising a combination of the same and different enhancer sequences is non-sequentially arranged at 5′ to said core promoter in the recombinant polynucleotide. In some embodiments, each of said at least two enhancer sequences comprises (iii), wherein each of said plurality of enhancers comprising a combination of the same and different enhancer sequences is non-sequentially arranged at 5′ to said core promoter and at 3′ to said plurality of binding sites for one or more TFs, if present, in the recombinant polynucleotide. In some embodiments, said plurality of enhancers comprises at least two EBS, C/EBP, ARE, DRE, NFκB, GC-box, UN5CL, BOP1, RTN4RL2, ARNTL2, AGR2, LHX2, TRNP1, MU5AC, or DOK4 enhancer sequences.

In some embodiments, expression of said ORF is increased when said recombinant polynucleotide is introduced to cancer cells compared to non-cancer cells. In some embodiments, expression of said ORF is increased in a first plurality of cancer cells when said recombinant polynucleotide is introduced to said first plurality of cancer cells compared to a second plurality of cancer cells, wherein said first plurality of cancer cells and said second plurality of cancer cells are different types of cancer cells. In some embodiments, said cancer cells comprise malignant cancer cells. In some embodiments, said cancer cells comprise lung cancer cells, colorectal cancer cells, breast cancer cells, or hepatocellular carcinoma cells. In some embodiments, said cancer cells comprise cells associated with colorectal cancer, hepatocellular carcinoma, lung cancer, liver cancer, breast cancer, prostate cancer, cervix cancer, uterus cancer, pancreas cancer, kidney cancer, stomach cancer, bladder cancer, ovary cancer, brain cancer, head and neck cancer, eye cancer, mouth cancer, throat cancer, esophagus cancer, chest cancer, bone cancer, rectum or other gastrointestinal tract organ cancer, spleen cancer, skeletal muscle cancer, subcutaneous tissue cancer, testicles or other reproductive organ cancer, skin cancer, thyroid cancer, blood cancer, or lymph nodes cancer. In some embodiments, said cancer cells comprise cells associated with two or more cancers comprising colorectal cancer, hepatocellular carcinoma, lung cancer, liver cancer, breast cancer, prostate cancer, cervix cancer, uterus cancer, pancreas cancer, kidney cancer, stomach cancer, bladder cancer, ovary cancer, brain cancer, head and neck cancer, eye cancer, mouth cancer, throat cancer, esophagus cancer, chest cancer, bone cancer, rectum or other gastrointestinal tract organ cancer, spleen cancer, skeletal muscle cancer, subcutaneous tissue cancer, testicles or other reproductive organ cancer, skin cancer, thyroid cancer, blood cancer, or lymph nodes cancer.

In some embodiments, said core promoter, said plurality of binding sites for one or more transcription factors (TFs), said plurality of enhancers, or said recombinant polynucleotide comprises a sequence from Table 1A, Table 1B, or Table 1C.

In some aspects, provided herein is a recombinant polynucleotide comprising any of the sequences from Table 1A, Table 1B, or Table 1C.

In some aspects, provided herein is a recombinant polynucleotide comprising a human alpha-fetoprotein (AFP) promoter sequence comprising a plurality of HNF-1A TF binding sites, wherein each HNF-1A binding site comprises the sequence 5′-GTTAATTATTAAC-3′ (SEQ ID NO: 128).

In some aspects, provided herein is a vector comprising any of the recombinant polynucleotide described herein. In some aspects, provided herein is a pharmaceutical composition comprising any of the recombinant polynucleotide described herein or any the vector described herein and a pharmaceutically acceptable excipient, carrier, or diluents. In some aspects, provided herein is a lipid nanoparticle (LNP) comprising any of the recombinant polynucleotide described herein, any of the vector described herein, or any of the pharmaceutical composition described herein. In some aspects, provided herein is a cell comprising any the recombinant polynucleotide described herein, any of the vector described herein, any of the pharmaceutical composition described herein, or any of the LNP described herein.

In some aspects, provided herein is a method of selectively expressing a reporter protein in a cancer or tumor cell, comprising contacting said tumor cell with any of the recombinant polynucleotide described herein, any of the vector described herein, any of the pharmaceutical composition described herein, or any of the LNP described herein, wherein the recombinant polynucleotide further comprises an open reading frame (ORF) encoding said reporter protein, wherein said ORF is operatively linked to said synthetic promoter.

In some aspects, provided herein is a method comprising: (a) administering to a subject any of the pharmaceutical composition described herein; or a composition any of the recombinant polynucleotide described herein, any of the vector described herein, or any of the LNP described herein; wherein the recombinant polynucleotide further comprises an open reading frame (ORF) encoding a reporter protein, wherein said ORF is operatively linked to a synthetic promoter in said recombinant polynucleotide, and (b) detecting said reporter protein, wherein said pharmaceutical composition or said composition induces expression of said reporter protein preferentially in diseased cells in said subject compared to in non-disease cells, and wherein a relative ratio of said reporter protein expressed in said diseased cells over said non-diseased cells is greater than 1.0. In some embodiments, said relative ratio of said reporter protein expressed in said diseased cells over said non-diseased cells is greater than 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, or about 15.0, 20.0, 25.0, 30.0, 35.0, 40.0, 45.0, 50.0, 55.0, 60.0, 65.0, 70.0, 75.0, 80.0, 85.0, 90.0, 95.0, or about 100.0.

In some aspects, provided herein is a method for treating a subject having or suspected of having a disease, comprising administering to said subject any of the pharmaceutical composition described herein; or a composition any of the recombinant polynucleotide described herein, any of the vector described herein, or any of the LNP described herein; wherein the recombinant polynucleotide further comprises an open reading frame (ORF) encoding a therapeutic protein, wherein said ORF is operatively linked to a synthetic promoter in said recombinant polynucleotide, wherein said pharmaceutical composition or said composition induces expression of said therapeutic protein preferentially in diseased cells in said subject compared to in non-disease cells, and wherein a relative ratio of said therapeutic protein expressed in said diseased cells over said non-diseased cells is greater than 1.0.

In some embodiments, said diseased cells comprise a cancer or tumor cell. In some embodiments, said cancer or tumor cell is associated with colorectal cancer (CRC), hepatocellular carcinoma, lung cancer, liver cancer, breast cancer, prostate cancer, cervix cancer, uterus cancer, pancreas cancer, kidney cancer, stomach cancer, bladder cancer, ovary cancer, brain cancer, head and neck cancer, eye cancer, mouth cancer, throat cancer, esophagus cancer, chest cancer, bone cancer, rectum or other gastrointestinal tract organ cancer, spleen cancer, skeletal muscle cancer, subcutaneous tissue cancer, testicles or other reproductive organ cancer, skin cancer, thyroid cancer, blood cancer, or lymph nodes cancer.

In some aspects, provided herein is a method comprising: (a) administering to a subject any of the pharmaceutical composition described herein; or a composition any of the recombinant polynucleotide described herein, any of the vector described herein, or any of the LNP described herein; wherein the recombinant polynucleotide further comprises an open reading frame (ORF) encoding a reporter protein, wherein said ORF is operatively linked to a synthetic promoter in said recombinant polynucleotide, and (b) localizing a tumor or an absence thereof in a body of said subject via expression of said reporter protein using an imaging technique performed on said body of said subject.

In some aspects, provided herein is a method comprising: (a) introducing to a subject suspected of having a cancer via intravenous administration any of the pharmaceutical composition described herein; or a composition any of the recombinant polynucleotide described herein, any of the vector described herein, or any of the LNP described herein; wherein said recombinant polynucleotide further comprises an open reading frame (ORF) encoding a reporter protein, wherein said ORF is operatively linked to a synthetic promoter in said recombinant polynucleotide, and (b) detecting said reporter protein from said subject.

In some aspects, provided herein is a method comprising: (a) introducing to a subject suspected of having a cancer via intravenous administration a plurality of recombinant polynucleotides, wherein: said plurality of recombinant polynucleotides comprises a plurality of different promoters of genes overexpressed in a tumor cell versus a normal tissue or functional fragments thereof operably linked to genes encoding reporter proteins, wherein said plurality of different promoters of genes overexpressed in said tumor cell versus said normal tissue drive expression of said corresponding reporter proteins in a cell affected by said cancer, wherein said DNA molecules are selected from the group consisting of nanoplasmids and linear double-stranded DNA molecules; and (b) detecting said reporter proteins from said subject.

TABLE 1A
Sequences of engineered promoters according to the disclosure

| SEQ | EA | | |
|---|---|---|---|
| ID | RLI. | | |
| NO: | ID | Name | Regulatory element sequence (nucleotide) |
| 1 | PL1 | 1- | ggcctaactggccggtaccacatcggctatgctgctgctatgcgagcgtcagtattt |
| | 009 | TRPS1_ | tatctttgatcagctattttatctttagtatcgtattttatctttctcatcgtattt |
| | | v22- | tatctttatccgattattttatctttcagcagttattttatctttggtacctgcgct |
| | | coreBIRC5- | cccgacatgccccgcggcgcgccattaaccgccagatttgagtcgcgggacccgttg |
| | | FLUC | gcagaggtgggctagcctcgaggatatcaagatctggcctcggcggccaagcttggc |
| | | | aatccggtactgttggtaaagccacc |
| 2 | PL1 | 2- | ggcctaactggccggtaccagctcatgcctatccgattagcttatcttttgaccaga |
| | 010 | TRPS1_ | gctagcttatctttctaactcgcatagcttatcttttgcaagctactagcttatctt |
| | | v9- | tcgatgctcattagcttatctttagacgtactctagcttatctttggtacctgcgct |
| | | coreBIRC5- | cccgacatgccccgcggcgcgccattaaccgccagatttgagtcgcgggacccgttg |
| | | FLUC | gcagaggtgggctagcctcgaggatatcaagatctggcctcggcggccaagcttggc |
| | | | aatccggtactgttggtaaagccacc |
| 3 | PL1 | 3- | ggcctaactggccggtaccatcactgctgaggtacagatgcacgatgtagctgagcg |
| | 011 | MNX1_ | acagtatagtgcacagtgagtcattatgatacgtgtcattatcaccattgtcattat |
| | | v18- | tagacgtgtcattatctgctatgtcattatgctacaggtcattatggtacctgcgct |
| | | coreBIRC5- | cccgacatgccccgcggcgcgccattaaccgccagatttgagtcgcgggacccgttg |
| | | FLUC | gcagaggtgggctagcctcgaggatatcaagatctggcctcggcggccaagcttggc |
| | | | aatccggtactgttggtaaagccacc |
| 4 | PL1 | 4- | ggcctaactggccggtacccagcagtcattatacgtcgcctaaatcgagatgctgta |
| | 012 | TWIST1_ | ctgatctatattccagatgttttcaattccagatgttttacattccagatgttttac |
| | | v3- | attccagatgtttctcattccagatgttttgaattccagatgtttggtacctgcgct |
| | | coreBIRC5- | cccgacatgccccgcggcgcgccattaaccgccagatttgagtcgcgggacccgttg |
| | | FLUC | gcagaggtgggctagcctcgaggatatcaagatctggcctcggcggccaagcttggc |
| | | | aatccggtactgttggtaaagccacc |
| 5 | PL1 | 5- | ggcctaactggccggtaccctgagcgacagtatagtgcacagtgacattacagatgt |
| | 013 | TWIST1_ | ttacgacgaattacagatgtttctcatcgattacagatgtttcagctcaattacaga |
| | | v18- | tgtttgctgctgattacagatgtttaccagagattacagatgtttggtacctgcgct |
| | | coreBIRC5- | cccgacatgccccgcggcgcgccattaaccgccagatttgagtcgcgggacccgttg |
| | | FLUC | gcagaggtgggctagcctcgaggatatcaagatctggcctcggcggccaagcttggc |
| | | | aatccggtactgttggtaaagccacc |
| 6 | PL1 | 6- | ggcctaactggccggtacccgatgtagctgagcgacagtatagtgcacagtgactgc |
| | 014 | HOXA1_ | agcagtcattatacgtcgcctaaatcgagatgctgtactgatctataaggatcggta |
| | | v8- | atgacgtaatgacgtaatgacgtaatgacgtaatgacgtaatgacggtacctgcgct |
| | | coreBIRC5- | cccgacatgccccgcggcgcgccattaaccgccagatttgagtcgcgggacccgttg |
| | | FLUC | gcagaggtgggctagcctcgaggatatcaagatctggcctcggcggccaagcttggc |
| | | | aatccggtactgttggtaaagccacc |
| 7 | PL1 | 7- | ggcctaactggccggtaccagctgagcgacagtatagtgcacagtgactgcagcagt |
| | 015 | HOXC10_ | cattatacgtcgcctaaatcgagatgctgtactgatctataagtcgtaaactgtcgt |
| | | v24- | aaactgtcgtaaactgtcgtaaactgtcgtaaactgtcgtaaactggtacctgcgct |
| | | coreBIRC5- | cccgacatgccccgcggcgcgccattaaccgccagatttgagtcgcgggacccgttg |
| | | FLUC | gcagaggtgggctagcctcgaggatatcaagatctggcctcggcggccaagcttggc |
| | | | aatccggtactgttggtaaagccacc |
| 8 | PL1 | 8- | ggcctaactggccggtacctgtagctgagcgacagtatagtgcacagtgactgcagc |
| | 016 | HOXC10_ | agtcattgtcgtaaattgagtatcgtcgtaaattgacgaacgtcgtaaattagcgac |
| | | v14- | agtcgtaaattagtacctgtcgtaaattactctgcgtcgtaaattggtacctgcgct |
| | | coreBIRC5- | cccgacatgccccgcggcgcgccattaaccgccagatttgagtcgcgggacccgttg |
| | | FLUC | gcagaggtgggctagcctcgaggatatcaagatctggcctcggcggccaagcttggc |
| | | | aatccggtactgttggtaaagccacc |
| 9 | PL1 | 9- | ggcctaactggccggtaccatccgatgtgcctgacgaactcatttctaatctatcga |
| | 017 | GATA1_ | tgtagctttctaatctatgcagtcattattctaatctattcgcaatctattctaatc |
| | | v1- | tatcttctaactcttctaatctattgctacagctttctaatctatggtacctgcgct |
| | | coreBIRC5- | cccgacatgccccgcggcgcgccattaaccgccagatttgagtcgcgggacccgttg |
| | | FLUC | gcagaggtgggctagcctcgaggatatcaagatctggcctcggcggccaagcttggc |
| | | | aatccggtactgttggtaaagccacc |
| 10 | PL1 | 10- | ggcctaactggccggtaccgcacagtgactgcagcagtcattatacgtcgcctaaat |
| | 018 | NFIC_ | cgagatgctgtactgatctatttcttggcagatgattcttggcagatcgttcttggc |
| | | v15- | agagcattcttggcagaggtttcttggcagactcttcttggcagaggtacctgcgct |
| | | coreBIRC5- | cccgacatgccccgcggcgcgccattaaccgccagatttgagtcgcgggacccgttg |
| | | FLUC | gcagaggtgggctagcctcgaggatatcaagatctggcctcggcggccaagcttggc |
| | | | aatccggtactgttggtaaagccacc |
| 11 | PL1 | 11- | ggcctaactggccggtaccgtgcaccattagtacctgatcagcgatgctcatctcga |
| | 019 | EN2_v7- | cctgatcggtacaacttctcacggaggcttctaactcgccgcaattataacgcaatt |
| | | coreBIR | attccgcaattactacgcaattacctcgcaattaactcgcaattaggtacctgcgct |
| | | C5- | cccgacatgccccgcggcgcgccattaaccgccagatttgagtcgcgggacccgttg |
| | | FLUC | gcagaggtgggctagcctcgaggatatcaagatctggcctcggcggccaagcttggc |
| | | | aatccggtactgttggtaaagccacc |
| 12 | PL1 | 12- | ggcctaactggccggtaccacatcggctatgctgctgctaatgccacgtcaccacat |
| | 020 | CREB3L1_ | cgacatgccacgtcaccatcatgccatgccacgtcaccactgcaagatgccacgtca |
| | | v6- | ccacagtataatgccacgtcaccaagttactatgccacgtcaccaggtacctgcgct |
| | | coreBIR | cccgacatgccccgcggcgcgccattaaccgccagatttgagtcgcgggacccgttg |
| | | C5- | gcagaggtgggctagcctcgaggatatcaagatctggcctcggcggccaagcttggc |
| | | FLUC | aatccggtactgttggtaaagccacc |
| 13 | PL1 | 13- | ggcctaactggccggtaccccccaaatcaccccccccccaccgtaaagtccccaaat |
| | 021 | RREB1_ | caccccccccccaaggtaagacccccaaatcacccccccccccgtcgcctaacccca |
| | | v17- | aatcacccccccccctactctgctcccccaaatcaccccccccccggtacctgcgct |
| | | coreBIRC5- | cccgacatgccccgcggcgcgccattaaccgccagatttgagtcgcgggacccgttg |
| | | FLUC | gcagaggtgggctagcctcgaggatatcaagatctggcctcggcggccaagcttggc |
| | | | aatccggtactgttggtaaagccacc |
| 14 | PL1 | 14- | ggcctaactggccggtaccgaccgtaaagtggtgtgcaccattgaaacttgagctta |
| | 022 | SIX4_ | caccatcgaaacttgagcgtatcgcatcgaaacttgagcggtacagatggaaacttg |
| | | v9- | agcaccattagtagaaacttgagcagcgacagtagaaacttgagcggtacctgcgct |
| | | coreBIRC5- | cccgacatgccccgcggcgcgccattaaccgccagatttgagtcgcgggacccgttg |
| | | FLUC | gcagaggtgggctagcctcgaggatatcaagatctggcctcggcggccaagcttggc |
| | | | aatccggtactgttggtaaagccacc |
| 15 | PL1 | 15- | ggcctaactggccggtacctgcacagtgactgcagcagtcgggcgtgcgctcccgac |
| | 023 | SURV_ | tagcccagggcgtgcgctcccgactagccccgggcgtgcgctcccgactagccctgg |
| | | v11- | gcgtgcgctcccgactagccccgggcgtgcgctcccgactagcccggtacctgcgct |
| | | coreBIRC5- | cccgacatgccccgcggcgcgccattaaccgccagatttgagtcgcgggacccgttg |
| | | FLUC | gcagaggtgggctagcctcgaggatatcaagatctggcctcggcggccaagcttggc |
| | | | aatccggtactgttggtaaagccacc |
| 16 | PL1 | 16- | ggcctaactggccggtaccaggatcgactagaagtcgcagattagacgacgatacgt |
| | 024 | TCF7_v3 | actactctgctcctagacgtatcctttgatgtaaatcctttgatgtcaatcctttga |
| | | coreBIRC5- | tgttaatcctttgatgttagtcctttgatgtctgtcctttgatgtggtacctgcgct |
| | | FLUC | cccgacatgccccgcggcgcgccattaaccgccagatttgagtcgcgggacccgttg |
| | | | gcagaggtgggctagcctcgaggatatcaagatctggcctcggcggccaagcttggc |
| | | | aatccggtactgttggtaaagccacc |
| 17 | PL1 | 17- | ggcctaactggccggtacctgagcgacagtatagtgcacagtgactgcagcagtcat |
| | 025 | TCF7L1_ | tatacgtcgcctaaaagacatcaaaggtccagacatcaaaggtacagacatcaaagg |
| | | v19- | ggaagacatcaaagggacagacatcaaaggtgcagacatcaaaggggtacctgcgct |
| | | coreBIRC5- | cccgacatgccccgcggcgcgccattaaccgccagatttgagtcgcgggacccgttg |
| | | FLUC | gcagaggtgggctagcctcgaggatatcaagatctggcctcggcggccaagcttggc |
| | | | aatccggtactgttggtaaagccacc |
| 18 | PL 1 | 18- | ggcctaactggccggtaccatgcacgatgtagctgagaaacatcaaaggacgcaacg |
| | 026 | TCF7L1_ | ccaaacatcaaaggagcctacacgaaacatcaaagggacgctgctaaaacatcaaag |
| | | v5- | gctacacgaccaaacatcaaagggccttacaccaaacatcaaaggggtacctgcgct |
| | | coreBIRC5- | cccgacatgccccgcggcgcgccattaaccgccagatttgagtcgcgggacccgttg |
| | | FLUC | gcagaggtgggctagcctcgaggatatcaagatctggcctcggcggccaagcttggc |
| | | | aatccggtactgttggtaaagccacc |
| 19 | PL1 | CREB3L1_ | GAATTCTAGTGCACAGTGACTGCAGCAATGCCACGTCAACATCATGCCATGCCACGT |
| | 030 | v14 | CAACACCTACACATGCCACGTCAACAACCAGAGATGCCACGTCAACACTAGCATATG |
| | | | CCACGTCAACATAAGGATATGCCACGTCAACAGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 20 | PL1 | EN2_v7 | GAATTCGTGCACCATTAGTACCTGATCAGCGATGCTCATCTCGACCTGATCGGTACA |
| | 031 | | ACTTCTCACGGAGGCTTCTAACTCGCCGCAATTATAACGCAATTATTCCGCAATTAC |
| | | | TACGCAATTACCTCGCAATTAACTCGCAATTAGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 21 | PL1 | ETV4_ | ggcctaacgaattcgacgctgctacagctcagcctacacgaccgtaaagtggtgtgc |
| | 032 | v14 | acaccggaaatgagtatagaccggaaatggccttacaccggaaatgcagctcaaccg |
| | | | gaaatgactgcagaccggaaatgcgctgctaccggaaatgggtacctgcgctcccga |
| | | | catgccccgcggcgcgccattaaccgccagatttgagtcgcgggacccgttggcaga |
| | | | ggtgggctagcctcgaggatatcaagatctggcctcggcggccaagcttggcaatcc |
| | | | ggtactgttggtaaagccaccatggtggcc |
| 22 | PL1 | ETV4_ | ggcctaactggccgaattctgagcgacagtatagtgcacagtgactgcagcagtcat |
| | 033 | v2 | tatacgtaccggaagtgtgtgcctaccggaagtgctatgcgaccggaagtgtagacg |
| | | | aaccggaagtgcagattaaccggaagtggctgctaaccggaagtgggtacctgcgct |
| | | | cccgacatgccccgcggcgcgccattaaccgccagatttgagtcgcgggacccgttg |
| | | | gcagaggtgggctagcctcgaggatatcaagatctggcctcggcggccaagcttggc |
| | | | aatccggtactgttggtaaagccacc |
| 23 | PL1 | MYCN_ | GAATTCGTGCACCATTAGTACCTGATCAGCGATGCTCATCTCGACCTGATCGGTACA |
| | 034 | v22 | ACTTCTCACGGAGGCTTCTAACTCGCCGCAATTATAACGCAATTATTCCGCAATTAC |
| | | | TACGCAATTACCTCGCAATTAACTCGCAATTAGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 24 | PL1 | PAX8_ | GAATTCGTCATTATACGTCGCGTCATGCATGACTGCCTGAGCGGTCATGCATGACTG |
| | 035 | v18 | CTACTCAAGTCATGCATGACTGCGACCAGAGTCATGCATGACTGCCGCCTAAGTCAT |
| | | | GCATGACTGCCTCTGCTGTCATGCATGACTGCGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 25 | PL1 | PITX2_ | GAATTCAAGTCGCAGATTAGACGACGATACGTACTACTCTGCTCCTAGACGTACTCA |
| | 036 | v22 | AGTATATTAATCCAGTGACCATTAATCCACTCATGCTTAATCCAATAACTGTTAATC |
| | | | CAGTATCGCTTAATCCACTACAGCTTAATCCAGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 26 | PL1 | SIX2_ | ggcctaactggccgaattccagatgcacgatgtagctgagcgacagtaaactgtaac |
| | 037 | v7 | ctgatacagcaactgtaacctgataccctaactgtaacctgatacgataactgtaac |
| | | | ctgatacaaaaactgtaacctgatacggcaactgtaacctgatacggtacctgcgct |
| | | | cccgacatgccccgcggcgcgccattaaccgccagatttgagtcgcgggacccgttg |
| | | | gcagaggtgggctagcctcgaggatatcaagatctggcctcggcggccaagcttggc |
| | | | aatccggtactgttggtaaagccacc |
| 27 | PL1 | SOX11_ | ggcctaactggccgaattcgactgcagcagtcattatacgtcgcctaaatcggagaa |
| | 038 | v2 | caaaggatggtgtggagaacaaaggataactgagagaacaaaggaaggatcggagaa |
| | | | caaaggaactgctggagaacaaaggatatagtggagaacaaaggaggtacctgcgct |
| | | | cccgacatgccccgcggcgcgccattaaccgccagatttgagtcgcgggacccgttg |
| | | | gcagaggtgggctagcctcgaggatatcaagatctggcctcggcggccaagcttggc |
| | | | aatccggtactgttggtaaagccacc |
| 28 | PL1 | TCF7_ | ggcctaactggccgaattcctgagcgacagtatagtgcacagtgactgcagcagtca |
| | 039 | v2 | ttcctttgatgtacgcaactcctttgatgtctatgcgtcctttgatgttaaggattc |
| | | | ctttgatgtaggtacatcctttgatgtccgtaaatcctttgatgtggtacctgcgct |
| | | | cccgacatgccccgcggcgcgccattaaccgccagatttgagtcgcgggacccgttg |
| | | | gcagaggtgggctagcctcgaggatatcaagatctggcctcggcggccaagcttggc |
| | | | aatccggtactgttggtaaagccacc |
| 29 | PL1 | TCF7_ | GAATTCAGGATCGACTAGAAGTCGCAGATTAGACGACGATACGTACTACTCTGCTCC |
| | 040 | v3 | TAGACGTATCCTTTGATGTAAATCCTTTGATGTCAATCCTTTGATGTTAATCCTTTG |
| | | | ATGTTAGTCCTTTGATGTCTGTCCTTTGATGTGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 30 | PL1 | TFDP1_ | ggcctaactggccgaattccaagactgcaagctacgtgtgaccagagccgataactg |
| | 041 | v6 | agggcgggaacgcgcaacggggcgggaacgatgctgtgggcgggaacgacagctcgg |
| | | | gcgggaacgctctgctgggcgggaacggctcctagggcgggaacgggtacctgcgct |
| | | | cccgacatgccccgcggcgcgccattaaccgccagatttgagtcgcgggacccgttg |
| | | | gcagaggtgggctagcctcgaggatatcaagatctggcctcggcggccaagcttggc |
| | | | aatccggtactgttggtaaagccacc |
| 31 | PL1 | E2F7_ | GAATTCAGGATCGACTAGAAGTCGCAGATTAGACGACGATACGTACTACTCTGCTCC |
| | 042 | v11 | TAGACGTATCCTTTGATGTAAATCCTTTGATGTCAATCCTTTGATGTTAATCCTTTG |
| | | | ATGTTAGTCCTTTGATGTCTGTCCTTTGATGTGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 32 | PL1 | E2F7_ | GAATTCAGGTAAGTTTCCCGCCAAAATGTGACCAGAGTTTCCCGCCAAAATGACGAA |
| | 043 | v13 | CTCGTTTCCCGCCAAAAATGTAGCTGAGTTTCCCGCCAAAACATAGTTACTGTTTCC |
| | | | CGCCAAAACCTAAATCGAGTTTCCCGCCAAAAGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 33 | PL1 | FOXA3_ | GAATTCTGCTATGCGAGCGTCAGCTCATGCCTATCCGATGTGCCTATGTAAACATAA |
| | 044 | v2 | GAGCCGATGTAAACATATAAGGATATGTAAACATATAGACGAATGTAAACATAGAGG |
| | | | TACATGTAAACATAACACGACATGTAAACATAGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 34 | PL1 | GLIS3_ | GAATTCTACAGCTCAGCCTACACGACCGTAAAGTGGTGTGCACCATTGACCCCCCAC |
| | 045 | v7 | AAAGCAGGACCCCCCACAAAGCGAGACCCCCCACAAAGGACGACCCCCCACAAAGCC |
| | | | TGACCCCCCACAAAGAGTGACCCCCCACAAAGGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 35 | PL1 | GLIS3_ | GAATTCAAGGTAGACCCCCCACTAAGCTCAAGTATAGACCCCCCACTAAGATAGTGC |
| | 046 | v9 | ACAGACCCCCCACTAAGTATCCGATGTGACCCCCCACTAAGCGCAACGCCTGACCCC |
| | | | CCACTAAGTCCTAGACGTGACCCCCCACTAAGGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 36 | PL1 | HOXC9_ | GAATTCAACTGAGTATCGCATCGCTCAAGATCAGTGGTCATAAATTAGCAGTCATTG |
| | 047 | v21 | TCATAAATTCCTGATCGGTGTCATAAATTGCCTAAATCGGTCATAAATTCAGCTCAT |
| | | | GCGTCATAAATTACGCTGCTACGTCATAAATTGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 37 | PL1 | NR2F6_ | GAATTCAGTATAGTGCACAGTGACTGCAGCAGTCATTATACGTCGCCGGGGTCAAAG |
| | 048 | v11 | GTCACCAGGGGTCAAAGGTCATCTGGGGTCAAAGGTCATTAGGGGTCAAAGGTCATA |
| | | | GGGGGTCAAAGGTCACGAGGGGTCAAAGGTCAGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 38 | PL1 | NR2F6_ | AATTCACATCGGCTATGCTGCTGCTACAGGTCAAAGGTCATTAGACGCAGGTCAAAG |
| | 049 | v18 | GTCACACAGTGCAGGTCAAAGGTCAAGGTACACAGGTCAAAGGTCACTGACGACAGG |
| | | | TCAAAGGTCACTCATCTCAGGTCAAAGGTCAGGTACCTGCGCTCCCGACATGCCCCG |
| | | | CGGCGCGCCATTAACCGCCAGATTTGAGTCGCGGGACCCGTTGGCAGAGGTGGGCTA |
| | | | GCCTCGAGGATATCAAGATCTGGCCTCGGCGGCCAAGCTTGGCAATCCGGTACTGTT |
| | | | GGTAAAGCCACC |
| 39 | PL1 | E2F3_ | GAATTCTGCACCATTAGTACCTGATCAGCGATGCTATTTTGGCGCCCAAATCATATT |
| | 050 | v11 | TTGGCGCCCAAATGACATTTTGGCGCCCAAATACAATTTTGGCGCCCAAATACGATT |
| | | | TTGGCGCCCAAATAGCATTTTGGCGCCCAAATGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 40 | PL1 | E2F4_ | GAATTCGGTACAACTTCTCACGGAGGCTTTTGGCGCCATTTCGACGATTTTTGGCGC |
| | 051 | v2 | CATTTACTCAAGTTTTGGCGCCATTTTAGTGCATTTTGGCGCCATTTCGCAATCTTT |
| | | | TGGCGCCATTTGGAGGCTTTTTGGCGCCATTTGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 41 | PL1 | EN2_v6 | GAATTCACGATACGTACTACTCTGCTCCTAGACGTACTCAAGTATAAGGTAAGACAT |
| | 052 | | AGTTACCGCAATTATAAGACACGCAATTACTAGAAGCGCAATTAACGTCGCCGCAAT |
| | | | TAGACTGCACGCAATTAGAATCTCCGCAATTAGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 42 | PL1 | FOXK1_ | GAATTCAAGTATAATGTAAACACGGCAGCATCGTCCAATGTAAACACGGCAAGACAT |
| | 053 | v9 | AGTAATGTAAACACGGCTCTCACGGAGAATGTAAACACGGCCTAGCATCGTAATGTA |
| | | | AACACGGCGATGCTCATCAATGTAAACACGGCGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 43 | PL1 | GRHL1_ | GAATTCAAGTCGCAGATTAGACGAAAAACCGGTTATGACGTACTCAAAAACCGGTTA |
| | 054 | v5 | TGAGATGCTGTAAAACCGGTTATTCCGACGCAAAAAACCGGTTATACGAACTCATAA |
| | | | AACCGGTTATAGCTCAGCCTAAAACCGGTTATGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 44 | PL1 | HOXB9_ | GAATTCTGACTGCAGCAGTCATTATACGTCGCCTAAATCGAGATGCTGTACGTCGTA |
| | 055 | v6 | AATTCACGACCGTCGTAAATTCGATAACGTCGTAAATTCTAGCATGTCGTAAATTTG |
| | | | CAGCAGTCGTAAATTAGATTAGGTCGTAAATTGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 45 | PL1 | MNX1_ | GAATTCATTAGACGACGATACGTACTACTCTGCTCCTAGACGTACTCAAGTATAAGG |
| | 056 | v10 | TAAGACGCAATTATTGCACAGGCAATTATTCAGCCTGCAATTATCTACAGCGCAATT |
| | | | ATCTGATCAGCAATTATGATACGTGCAATTATGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 46 | PL1 | MYC_ | GAATTCACTCTGCTCCTAGACGTACTCAAGTATAAGGTAGGACACGTGCCCGATGCA |
| | 057 | v22 | CGGACACGTGCCCCCGTAAAGGACACGTGCCCTAAATCGGGACACGTGCCCTAGACG |
| | | | TGGACACGTGCCCGACTAGAGGACACGTGCCCGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 47 | PL1 | OTX1_ | GAATTCCACAGTGACTGCAGCAGTCATTATACGTCGCCTAAATCGAGATGCTGTACT |
| | 058 | v14 | GATCTATTAAGCCGCGTACTCTTAAGCCGGTCATTATTAAGCCGCTATAAGTTAAGC |
| | | | CGCAACGCCTTAAGCCGACGACCGTTAAGCCGGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 48 | PL1 | PITX2_ | GAATTCTCGGCTATGCTGCTGCTATGCGAGCGTCAGCTCATGCCTATCCGATGTGCC |
| | 059 | v19 | TGACGAACTCATCGACGCTGCTACAGCTAATCCTATGCTAATCCTAACCTAATCCTA |
| | | | CCCTAATCCTAGCCTAATCCTTGCCTAATCCTGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 49 | PL1 | RUNX1_ | GAATTCTGTACTGATCTATAAGGATCGACTAGAAGTCGCAGATTAGTATGTGGTTTA |
| | 060 | v22 | GTACCTGTATGTGGTTTTCGCAATGTATGTGGTTTATGCTGCGTATGTGGTTTAGCA |
| | | | GTCGTATGTGGTTTGAGCGTCGTATGTGGTTTGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 50 | PL1 | RUNX1_ | GAATTCCTGCAGCAGTCATTATACGTCGCCTAAATCGAGATGCTGTACTGATCTATA |
| | 061 | v23 | AGGATCGAGTATGTGGTTTATCGTATGTGGTTTGTAGTATGTGGTTTCTGGTATGTG |
| | | | GTTTTGTGTATGTGGTTTCCAGTATGTGGTTTGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 51 | PL1 | SHOX2_ | GAATTCCACGATGTAGCTGAGCGACAGTATAGTGCACAGTGACTGCAGCCAATTAAC |
| | 062 | v5 | TGACGAACTCCAATTAAATCAGTGATCCCAATTAATGCAAGCTACCCAATTAATATG |
| | | | CTGCTGCCAATTAACATCGGCTATCCAATTAAGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 52 | PL1 | SHOX2_ | GAATTCTTAGTACCTGATCAGCGATGCTCATCTCGACCTGATCGGTACTCAATTAAT |
| | 063 | v21 | GTACTGATCTCAATTAAGTCGCCTAAATCAATTAACGTACTACTCTCAATTAAGATC |
| | | | GGTACATCAATTAAAAGTCGCAGATCAATTAAGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 53 | PL1 | SIX4_ | GAATTCCTACGTGTGACCAGAGCCGATAACTGAGTATCGCATCGCTCAAGATCAGTG |
| | 064 | v23 | ATCACTGCGAAATTTGAGCCCTGAAATTTGAGCCGAGAAATTTGAGCGCTGAAATTT |
| | | | GAGCCACGAAATTTGAGCTTAGAAATTTGAGCGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 54 | PL1 | TCF7_ | GAATTCGACCTGATCGGTACAACTTCTCACGGAGGCTTCTAACTCTCCTTTGATATA |
| | 065 | v10 | ACTCGCTCCTTTGATATAGCAGTCTCCTTTGATATCTCATCTTCCTTTGATATCTGT |
| | | | ACTTCCTTTGATATTGCTATGTCCTTTGATATGGTACCTGCGCTCCCGACATGCCCC |
| | | | GCGGCGCGCCATTAACCGCCAGATTTGAGTCGCGGGACCCGTTGGCAGAGGTGGGCT |
| | | | AGCCTCGAGGATATCAAGATCTGGCCTCGGCGGCCAAGCTTGGCAATCCGGTACTGT |
| | | | TGGTAAAGCCACC |
| 55 | PL1 | PL- | ggcctaactggccggtaccactagtggtgactcatgggtgactcatgggtgactcat |
| | 068 | 3XFOSL1- | ggtgatcatgctagcctcgaggatatcaagatcggtaccacctcttaacaatacgtt |
| | | coreAGR2_ | tcacaaatagttaaaaacatgcatactgaaaagcatacttttgcaatgttattttta |
| | | 2 | aaaacaaggaactctttaacccagggaagataatcacttggggaaaggaaggttcgt |
| | | | ttctgagttagcaacaagtaaatgcagcactagtgggtgggattgaggtgtgccctg |
| | | | gtgcataaatagagactcagctgtgctggcacactcagaagcttggaccgcatccta |
| | | | gccgccgactcacacaaggcaggtgggtgaggaaatccaggtaaggctcctgacagc |
| | | | agctttagaagggtacttgctggagtgaattcgggcctctgattaccggtgctagcc |
| | | | tcgaggatatcaagatctggcctcggcggccaagcttggcaatccggtactgttggt |
| | | | aaagccacc |
| 56 | PL1 | PL- | ggcctaactggccggtaccgatcttgatatcctcgaggctagcatgatcaccatgag |
| | 069 | revFOSL1- | tcacccatgagtcacccatgagtcacccatgagtcacccatgagtcacccatgagtc |
| | | coreAGR2_ | acccatgagtcacccatgagtcacccatgagtcaccactagtggtaccacctcttaa |
| | | 2 | caatacgtttcacaaatagttaaaaacatgcatactgaaaagcatacttttgcaatg |
| | | | ttatttttaaaaacaaggaactctttaacccagggaagataatcacttggggaaagg |
| | | | aaggttcgtttctgagttagcaacaagtaaatgcagcactagtgggtgggattgagg |
| | | | tgtgccctggtgcataaatagagactcagctgtgctggcacactcagaagcttggac |
| | | | cgcatcctagccgccgactcacacaaggcaggtgggtgaggaaatccaggtaaggct |
| | | | cctgacagcagctttagaagggtacttgctggagtgaattcgggcctctgattaccg |
| | | | gtgctagcctcgaggatatcaagatctggcctcggcggccaagcttggcaatccggt |
| | | | actgttggtaaagccacc |
| 57 | PL1 | PL- | ggcctaactggccggtaccgattcttgatatcctcgaggctagcatgatcaccatga |
| | 070 | revFOSL1- | gtcacccatgagtcacccatgagtcacccatgagtcacccatgagtcacccatgagt |
| | | coreCST1 | cacccatgagtcacccatgagtcacccatgagtcaccactagtggtaccgatcttga |
| | | | tatcctcgaggctagcatgatcaccatgagtcacccatgagtcacccatgagtcacc |
| | | | catgagtcacccatgagtcacccatgagtcacccatgagtcacccatgagtcaccca |
| | | | tgagtcaccactagtggtaccagtggtgggggagtgaaaagagagatggagaaagag |
| | | | gggatgggcagaaagaggaggaggagtcaggggcagggcatggaggtgggtggggct |
| | | | gggctgccaaagcaggataaatgcacacctgcctgctggtctgggctccctgcctcg |
| | | | ggctctcaccctcctctcctgcagctccagctttgtgcttctaccggtgctagcctc |
| | | | gaggatatcaagatctggcctcggcggccaagcttggcaatccggtactgttggtaa |
| | | | agccacc |
| 58 | PL1 | PL- | ggcctaactggccggtaccactagtgacgtcaccggaagtaagaaccggaagtatcg |
| | 071 | ETV4- | accggaagtagacaccggaagtactaaccggaagtaactaccggaagtatgcaccgg |
| | | coreCST | aagtagacgtctacgtaagtggtgggggagtgaaaagagagatggagaaagagggga |
| | | | tgggcagaaagaggaggaggagtcaggggcagggcatggaggtgggtggggctgggc |
| | | | tgccaaagcaggataaatgcacacctgcctgctggtctgggctccctgcctcgggct |
| | | | ctcaccctcctctcctgcagctccagctttgtgctctaccggtgctagcctcgagga |
| | | | tatcaagatctggcctcggcggccaagcttggcaatccggtactgttggtaaagcca |
| | | | CC |
| 59 | PL1 | PL- | ggcctaactggccggtaccactagtgacgtcaccggaagtaagaaccggaagtatcg |
| | 072 | ETV4- | accggaagtagacaccggaagtactaaccggaagtaactaccggaagtatgcaccgg |
| | | coreKIF | aagtagacgtctacgtaggcccgccccctttccttacgcggattggtagctgcaggc |
| | | | ttccctatctgattggccgaacgaacgcagcgcgtaatttaaaatattgtatctgta |
| | | | acaaagctgcacctcgtgggcggagttgtgctctgcggctgcgaaagtccagcttcg |
| | | | gcgactaggtgtgagtaagccagtatcccaggaggagcaagtggcacgtcttcgggt |
| | | | gagtgtgcggctgtgctggagcccgggttaccagctctttaccggtgctagcctcga |
| | | | ggatatcaagatctggcctcggcggccaagcttggcaatccggtactgttggtaaag |
| | | | ccacc |
| 60 | PL1 | PL- | ggcctaactggccggtacactagtgacgtcaccggaagtaagaaccggaagtatcga |
| | 073 | ETV4- | ccggaagtagacaccggaagtactaaccggaagtaactaccggaagtatgcaccgga |
| | | coreAGR2 | agtagacgtctacgtacatactgaaaagcatacttttgcaatgttatttttaaaaac |
| | | | aaggaactctttaacccagggaagataatcacttggggaaaggaaggttcgtttctg |
| | | | agttagcaacaagtaaatgcagcactagtgggtgggattgaggtgtgccctggtgca |
| | | | taaatagagactcagctgtgctggcacactcagaagcttggaccgcatcctagccgc |
| | | | cgactcacacaaggcaggtgggtgaggaaatccaggtaaggctcctgacagcagctt |
| | | | tagaagggtacttgctggagtgaattcgggcctctgattactagcctcgaggatatc |
| | | | aagatctggcctcggcggccaagcttggcaatccggtactgttggtaaagccacc |
| 61 | PL1 | PL- | ggcctaactggccggtaccactagtgacgtcaccggaagtaagaaccggaagtatcg |
| | 074 | ETV4- | accggaagtagacaccggaagtactaaccggaagtaactaccggaagtatgcaccgg |
| | | coreCEACAM | aagtagacgtctacgtaacccacgtgatgctgagaagtactcctgccctaggaagag |
| | | | actcagggcagagggaggaaggacagcagaccagacagtcacagcagccttgacaaa |
| | | | acgttcctggaactaccggtgctagcctcgaggatatcaagatctggcctcggcggc |
| | | | caagcttggcaatccggtactgttggtaaagccacc |
| 62 | PL1 | PL- | GGCCTAACTGGCCGGTACCACTAGTGACGTCACCGGAAGTAAGAACCGGAAGTATCG |
| | 075 | ETV4- | ACCGGAAGTAGACACCGGAAGTACTAACCGGAAGTAACTACCGGAAGTATGCACCGG |
| | | coreFAM111B | AAGTAGACGTCTACGTACGGGAAAAGTTCAGCTGAGAGATATAAAAGAGCAGTCTTT |
| | | | CCAGCACCTGCAAATCCAGAGCGGCGGGCACTGACGGGCACTTGCACCGTGTGGACA |
| | | | GACTCTCCGGTTCTGTGAGTGGTTTTTCTTTTCCCGGGTCGGACCTGGAGTTCTTAG |
| | | | GGGGATGGCTGAACCGGTGCTAGCCTCGAGGATATCAAGATCTGGCCTCGGCGGCCA |
| | | | AGCTTGGCAATCCGGTACTGTTGGTAAAGCCACC |
| 63 | PL1 | PL- | ggcctaactggccggtaccactagtgacgtcaccggaagtaagaaccggaagtatcg |
| | 076 | ETV4- | accggaagtagacaccggaagtactaaccggaagtaactaccggaagtatgcaccgg |
| | | Twist_ | aagtagacgtctacgtactgagcgacagtatagtgcacagtgacattacagatgttt |
| | | v18- | acgacgaattacagatgtttctcatcgattacagatgtttcagctcaattacagatg |
| | | coreCST | tttgctgctgattacagatgtttaccagagattacagatgttttacgtaagtggtgg |
| | | | gggagtgaaaagagagatggagaaagaggggatgggcagaaagaggaggaggagtca |
| | | | ggggcagggcatggaggtgggtggggctgggctgccaaagcaggataaatgcacacc |
| | | | tgcctgctggtctgggctccctgcctcgggctctcaccctcctctcctgcagctcca |
| | | | gctttgtgctctaccggtgctagcctcgaggatatcaagatctggcctcggcggcca |
| | | | agcttggcaatccggtactgttggtaaagccacc |
| 64 | PL1 | PL- | ACTAGTGACGTCACCGGAAGTAAGAACCGGAAGTATCGACCGGAAGTAGACACCGGA |
| | 077 | ETV4- | AGTACTAACCGGAAGTAACTACCGGAAGTATGCACCGGAAGTAGACGTCTACGTACT |
| | | Twist_ | GAGCGACAGTATAGTGCACAGTGACATTACAGATGTTTACGACGAATTACAGATGTT |
| | | v18- | TCTCATCGATTACAGATGTTTCAGCTCAATTACAGATGTTTGCTGCTGATTACAGAT |
| | | coreKIF | GTTTACCAGAGATTACAGATGTTTTACGTAGGCCCGCCCCCTTTCCTTACGCGGATT |
| | | | GGTAGCTGCAGGCTTCCCTATCTGATTGGCCGAACGAACGCAGCGCGTAATTTAAAA |
| | | | TATTGTATCTGTAACAAAGCTGCACCTCGTGGGCGGAGTTGTGCTCTGCGGCTGCGA |
| | | | AAGTCCAGCTTCGGCGACTAGGTGTGAGTAAGCCAGTATCCCAGGAGGAGCAAGTGG |
| | | | CACGTCTTCGGGTGAGTGTGCGGCTGTGCTGGAGCCCGGGTTACCAGCTCTTtaccg |
| | | | gtgctagcctcgaggatatcaagatctggcctcggcggccaagcttggcaatccggt |
| | | | actgttggtaaagccacc |
| 65 | PL1 | PL- | ggcctaactggccggtacactagtgacgtcaccggaagtaagaaccggaagtatcga |
| | 078 | ETV4- | ccggaagtagacaccggaagtactaaccggaagtaactaccggaagtatgcaccgga |
| | | Twist_ | agtagacgtctacgtactgagcgacagtatagtgcacagtgacattacagatgttta |
| | | v18- | cgacgaattacagatgtttctcatcgattacagatgtttcagctcaattacagatgt |
| | | coreAGR2 | ttgctgctgattacagatgtttaccagagattacagatgttttacgtacatactgaa |
| | | | aagcatacttttgcaatgttatttttaaaaacaaggaactctttaacccagggaaga |
| | | | taatcacttggggaaaggaaggttcgtttctgagttagcaacaagtaaatgcagcac |
| | | | tagtgggtgggattgaggtgtgccctggtgcataaatagagactcagctgtgctggc |
| | | | acactcagaagcttggaccgcatcctagccgccgactcacacaaggcaggtgggtga |
| | | | ggaaatccaggtaaggctcctgacagcagctttagaagggtacttgctggagtgaat |
| | | | tcgggcctctgattactagcctcgaggatatcaagatctggcctcggcggccaagct |
| | | | tggcaatccggtactgttggtaaagccacc |
| 66 | PL1 | PL- | ggcctaactggccggtaccactagtgacgtcaccggaagtaagaaccggaagtatcg |
| | 079 | ETV4- | accggaagtagacaccggaagtactaaccggaagtaactaccggaagtatgcaccgg |
| | | Twist_ | aagtagacgtctacgtactgagcgacagtatagtgcacagtgacattacagatgttt |
| | | v18- | acgacgaattacagatgtttctcatcgattacagatgtttcagctcaattacagatg |
| | | coreFAM111B | tttgctgctgattacagatgtttaccagagattacagatgttttacgtacgggaaaa |
| | | | gttcagctgagagatataaaagagcagtctttccagcacctgcaaatccagagcggc |
| | | | gggcactgacgggcacttgcaccgtgtggacagactctccggttctgtgagtggttt |
| | | | ttcttttcccgggtcggacctggagttcttagggggatggctgaaccggtgctagcc |
| | | | tcgaggatatcaagatctggcctcggcggccaagcttggcaatccggtactgttggt |
| | | | aaagccacc |
| 67 | PL1 | PL- | ggcctaactggccggtaccactagtgacgtcaccggaagtaagaaccggaagtatcg |
| | 080 | ETV4- | accggaagtagacaccggaagtactaaccggaagtaactaccggaagtatgcaccgg |
| | | Twist_ | aagtagacgtctacgtactgagcgacagtatagtgcacagtgacattacagatgttt |
| | | v18- | acgacgaattacagatgtttctcatcgattacagatgtttcagctcaattacagatg |
| | | coreCEACAM | tttgctgctgattacagatgtttaccagagattacagatgttttacgtaacccacgt |
| | | | gatgctgagaagtactcctgccctaggaagagactcagggcagagggaggaaggaca |
| | | | gcagaccagacagtcacagcagccttgacaaaacgttcctggaactaccggtgctag |
| | | | cctcgaggatatcaagatctggcctcggcggccaagcttggcaatccggtactgttg |
| | | | gtaaagccacc |
| 68 | PL1 | PL- | ggcctaactggccggtaccactagtgacgtctacgtactgagcgacagtatagtgca |
| | 081 | Twist_ | cagtgacattacagatgtttacgacgaattacagatgtttctcatcgattacagatg |
| | | v18- | tttcagctcaattacagatgtttgctgctgattacagatgtttaccagagattacag |
| | | coreCST | atgttttacgtaagtggtgggggagtgaaaagagagatggagaaagaggggatgggc |
| | | | agaaagaggaggaggagtcaggggcagggcatggaggtgggtggggctgggctgcca |
| | | | aagcaggataaatgcacacctgcctgctggtctgggctccctgcctcgggctctcac |
| | | | cctcctctcctgcagctccagctttgtgctctaccggtgctagcctcgaggatatca |
| | | | agatctggcctcggcggccaagcttggcaatccggtactgttggtaaagccacc |
| 69 | PL1 | PL- | ggcctaactggccggtaccactagtgacgtctacgtactgagcgacagtatagtgca |
| | 082 | Twist_ | cagtgacattacagatgtttacgacgaattacagatgtttctcatcgattacagatg |
| | | v18- | tttcagctcaattacagatgtttgctgctgattacagatgtttaccagagattacag |
| | | coreKIF | atgttttacgtaggcccgccccctttccttacgcggattggtagctgcaggcttccc |
| | | | tatctgattggccgaacgaacgcagcgcgtaatttaaaatattgtatctgtaacaaa |
| | | | gctgcacctcgtgggcggagttgtgctctgcggctgcgaaagtccagcttcggcgac |
| | | | taggtgtgagtaagccagtatcccaggaggagcaagtggcacgtcttcgggtgagtg |
| | | | tgcggctgtgctggagcccgggttaccagctctttaccggtgctagcctcgaggata |
| | | | tcaagatctggcctcggcggccaagcttggcaatccggtactgttggtaaagccacc |
| 70 | PL1 | PL- | ggcctaactggccggtaccactagtgacgtctacgtactgagcgacagtatagtgca |
| | 083 | Twist_ | cagtgacattacagatgtttacgacgaattacagatgtttctcatcgattacagatg |
| | | 8v1- | tttcagctcaattacagatgtttgctgctgattacagatgtttaccagagattacag |
| | | coreAGR2 | atgttttacgtacatactgaaaagcatacttttgcaatgttatttttaaaaacaagg |
| | | | aactctttaacccagggaagataatcacttggggaaaggaaggttcgtttctgagtt |
| | | | agcaacaagtaaatgcagcactagtgggtgggattgaggtgtgccctggtgcataaa |
| | | | tagagactcagctgtgctggcacactcagaagcttggaccgcatcctagccgccgac |
| | | | tcacacaaggcaggtgggtgaggaaatccaggtaaggctcctgacagcagctttaga |
| | | | agggtacttgctggagtgaattcgggcctctgattagctagcctcgaggatatcaag |
| | | | atctggcctcggcggccaagcttggcaatccggtactgttggtaaagccacc |
| 71 | PL1 | PL- | ggcctaactggccggtaccactagtgacgtcctgagcgacagtatagtgcacagtga |
| | 084 | Twist_ | cattacagatgtttacgacgaattacagatgtttctcatcgattacagatgtttcag |
| | | v18.2- | ctcaattacagatgtttgctgctgattacagatgtttaccagagattacagatgttt |
| | | coreKIF | gacgtctacgtaggcccgccccctttccttacgcggattggtagctgcaggcttccc |
| | | | tatctgattggccgaacgaacgcagcgcgtaatttaaaatattgtatctgtaacaaa |
| | | | gctgcacctcgtgggcggagttgtgctctgcggctgcgaaagtccagcttcggcgac |
| | | | taggtgtgagtaagccagtatcccaggaggagcaagtggcacgtcttcgggtgagtg |
| | | | tgcggctgtgctggagcccgggttaccagctctttaccggtgctagcctcgaggata |
| | | | tcaagatctggcctcggcggccaagcttggcaatccggtactgttggtaaagccacc |
| 72 | PL1 | PL- | ggcctaactggccggtaccactagtgacgtcctgagcgacagtatagtgcacagtga |
| | 085 | Twist_ | cattacagatgtttacgacgaattacagatgtttctcatcgattacagatgtttcag |
| | | v18.2- | ctcaattacagatgtttgctgctgattacagatgtttaccagagattacagatgttt |
| | | coreCST | gacgtctacgtactgatcagcgatgctcatctcgacctgatcggtacaacttctcac |
| | | | ggaggcttctaagtcattacatacgtagtcattactatacgtgtcattacagatgct |
| | | | gtcattacacgaactgtcattacgtactcagtcattactacgtaagtggtgggggag |
| | | | tgaaaagagagatggagaaagaggggatgggcagaaagaggaggaggagtcaggggc |
| | | | agggcatggaggtgggtggggctgggctgccaaagcaggataaatgcacacctgcct |
| | | | gctggtctgggctccctgcctcgggctctcaccctcctctcctgcagctccagcttt |
| | | | gtgctctaccggtgctagcctcgaggatatcaagatctggcctcggcggccaagctt |
| | | | ggcaatccggtactgttggtaaagccacc |
| 73 | PL1 | PL- | ggcctaactggccggtaccactagtgacgtctacgtactgagcgacagtatagtgca |
| | 086 | Twist_ | cagtgacattacagatgtttacgacgaattacagatgtttctcatcgattacagatg |
| | | v18- | tttcagctcaattacagatgtttgctgctgattacagatgtttaccagagattacag |
| | | coreFAM111B | atgttttacgtacgggaaaagttcagctgagagatataaaagagcagtctttccagc |
| | | | acctgcaaatccagagcggcgggcactgacgggcacttgcaccgtgtggacagactc |
| | | | tccggttctgtgagtggtttttcttttcccgggtcggacctggagttcttaggggga |
| | | | tggctgaaccggtgctagcctcgaggatatcaagatctggcctcggcggccaagctt |
| | | | ggcaatccggtactgttggtaaagccacc |
| 74 | PL1 | PL- | ggcctaactggccggtaccactagtgacgtctacgtactgagcgacagtatagtgca |
| | 087 | Twist_ | cagtgacattacagatgtttacgacgaattacagatgtttctcatcgattacagatg |
| | | v18- | tttcagctcaattacagatgtttgctgctgattacagatgtttaccagagattacag |
| | | coreCEACAM | atgttttacgtaacccacgtgatgctgagaagtactcctgccctaggaagagactca |
| | | | gggcagagggaggaaggacagcagaccagacagtcacagcagccttgacaaaacgtt |
| | | | cctggaactaccggtgctagcctcgaggatatcaagatctggcctcggcggccaagc |
| | | | ttggcaatccggtactgttggtaaagccacc |
| 75 | PL1 | PL- | ggcctaactggccggtaccactagtgacgtcctgagcgacagtatagtgcacagtga |
| | 088 | Twist_ | cattacagatgtttacgacgaattacagatgtttctcatcgattacagatgtttcag |
| | | v18.2- | ctcaattacagatgtttgctgctgattacagatgtttaccagagattacagatgttt |
| | | coreAGR2 | gacgtctacgtacatactgaaaagcatacttttgcaatgttatttttaaaaacaagg |
| | | | aactctttaacccagggaagataatcacttggggaaaggaaggttcgtttctgagtt |
| | | | agcaacaagtaaatgcagcactagtgggtgggattgaggtgtgccctggtgcataaa |
| | | | tagagactcagctgtgctggcacactcagaagcttggaccgcatcctagccgccgac |
| | | | tcacacaaggcaggtgggtgaggaaatccaggtaaggctcctgacagcagctttaga |
| | | | agggtacttgctggagtgaattcgggcctctgattagctagcctcgaggatatcaag |
| | | | atctggcctcggcggccaagcttggcaatccggtactgttggtaaagccacc |
| 76 | PL1 | PL- | ggcctaactggccggtaccactagtgacgtcctgagcgacagtatagtgcacagtga |
| | 089 | Twist_ | cattacagatgtttacgacgaattacagatgtttctcatcgattacagatgtttcag |
| | | v18.2- | ctcaattacagatgtttgctgctgattacagatgtttaccagagattacagatgttt |
| | | coreCEACAM | gacgtctacgtaacccacgtgatgctgagaagtactcctgccctaggaagagactca |
| | | | gggcagagggaggaaggacagcagaccagacagtcacagcagccttgacaaaacgtt |
| | | | cctggaactaccggtgctagcctcgaggatatcaagatctggcctcggcggccaagc |
| | | | ttggcaatccggtactgttggtaaagccacc |
| 77 | PL1 | PL- | ggcctaactggccggtaccactagtgacgtcctgagcgacagtatagtgcacagtga |
| | 090 | Twist_ | cattacagatgtttacgacgaattacagatgtttctcatcgattacagatgtttcag |
| | | v18.2- | ctcaattacagatgtttgctgctgattacagatgtttaccagagattacagatgttt |
| | | coreFAM111B | gacgtctacgtacgggaaaagttcagctgagagatataaaagagcagtctttccagc |
| | | | acctgcaaatccagagcggcgggcactgacgggcacttgcaccgtgtggacagactc |
| | | | tccggttctgtgagtggtttttcttttcccgggtcggacctggagttcttaggggga |
| | | | tggctgaaccggtgctagcctcgaggatatcaagatctggcctcggcggccaagctt |
| | | | ggcaatccggtactgttggtaaagccacc |
| 78 | PL1 | PL- | ggcctaactggccggtaccactagtgacgtcctgagcgacagtatagtgcacagtga |
| | 091 | Twist_ | cattacagatgtttacgacgaattacagatgtttctcatcgattacagatgtttcag |
| | | v18- | ctcaattacagatgtttgctgctgattacagatgtttaccagagattacagatgttt |
| | | HOXA1_ | gacgtctacgtactgatcagcgatgctcatctcgacctgatcggtacaacttctcac |
| | | v10- | ggaggcttctaagtcattacatacgtagtcattactatacgtgtcattacagatgct |
| | | coreKIF | gtcattacacgaactgtcattacgtactcagtcattactacgtaggcccgccccctt |
| | | | tccttacgcggattggtagctgcaggcttccctatctgattggccgaacgaacgcag |
| | | | cgcgtaatttaaaatattgtatctgtaacaaagctgcacctcgtgggcggagttgtg |
| | | | ctctgcggctgcgaaagtccagcttcggcgactaggtgtgagtaagccagtatccca |
| | | | ggaggagcaagtggcacgtcttcgggtgagtgtgcggctgtgctggagcccgggtta |
| | | | ccagctctttaccggtgctagcctcgaggatatcaagatctggcctcggcggccaag |
| | | | cttggcaatccggtactgttggtaaagccacc |
| 79 | PL1 | PL- | ggcctaactggccggtaccacactagtgacgtcctgagcgacagtatagtgcacagt |
| | 092 | Twist_ | gacattacagatgtttacgacgaattacagatgtttctcatcgattacagatgtttc |
| | | v18- | agctcaattacagatgtttgctgctgattacagatgtttaccagagattacagatgt |
| | | HOXA1_ | ttgacgtctacgtactgatcagcgatgctcatctcgacctgatcggtacaacttctc |
| | | v10- | acggaggcttctaagtcattacatacgtagtcattactatacgtgtcattacagatg |
| | | coreCST | ctgtcattacacgaactgtcattacgtactcagtcattactacgtacatactgaaaa |
| | | | gcatacttttgcaatgttatttttaaaaacaaggaactctttaacccagggaagata |
| | | | atcacttggggaaaggaaggttcgtttctgagttagcaacaagtaaatgcagcacta |
| | | | gtgggtgggattgaggtgtgccctggttaagtggtgggggagtgaaaagagagatgg |
| | | | agaaagaggggatgggcagaaagaggaggaggagtcaggggcagggcatggaggtgg |
| | | | gtggggctgggctgccaaagcaggataaatgcacacctgcctgctggtctgggctcc |
| | | | ctgcctcgggctctcaccctcctctcctgcagctccagctttgtgctctaccggtgc |
| | | | tagcctcgaggatatcaagatctggcctcggcggccaagcttggcaatccggtactg |
| | | | ttggtaaagccacc |
| 80 | PL1 | PL- | ggcctaactggccggtacaactagtgactcctttgatgtacgcaactcctttgatgt |
| | 093 | Twist_ | ctatgcgtcctttgatgttaaggattcctttgatgtaggtacatcctttgatgtccg |
| | | v18- | taaatcctttgatgtggtaccgtctactacctgatcaaacatgcccggacatgtcgt |
| | | HOXA1_ | aagacataaacatgcccggacatgtcctcgcaatctaacatgcccggacatgtcctc |
| | | v10- | gcaatctaacatgcccggacatgtctgcaagctacaacatgcccggacatgtcgtac |
| | | coreAGR2 | tcagtcattactacgtacatactgaaaagcatacttttgcaatgttatttttaaaaa |
| | | | caaggaactctttaacccagggaagataatcacttggggaaaggaaggttcgtttct |
| | | | gagttagcaacaagtaaatgcagcactagtgggtgggattgaggtgtgccctggtgc |
| | | | ataaatagagactcagctgtgctggcacactcagaagcttggaccgcatcctagccg |
| | | | ccgactcacacaaggcaggtgggtgaggaaatccaggtaaggctcctgacagcagct |
| | | | ttagaagggtacttgctggagtgaattcgggcctctgattactagcctcgaggatat |
| | | | caagatctggcctcggcggccaagcttggcaatccggtactgttggtaaagccacc |
| 81 | PL1 | PL- | ggcctaactggccggtaccactagtgacgtcctgagcgacagtatagtgcacagtga |
| | 094 | Twist_ | cattacagatgtttacgacgaattacagatgtttctcatcgattacagatgtttcag |
| | | v18- | ctcaattacagatgtttgctgctgattacagatgtttaccagagattacagatgttt |
| | | HOXA1_ | gacgtctacgtactgatcagcgatgctcatctcgacctgatcggtacaacttctcac |
| | | v10- | ggaggcttctaagtcattacatacgtagtcattactatacgtgtcattacagatgct |
| | | coreCEACAM | gtcattacacgaactgtcattacgtactcagtcattactacgtaacccacgtgatgc |
| | | | tgagaagtactcctgccctaggaagagactcagggcagagggaggaaggacagcaga |
| | | | ccagacagtcacagcagccttgacaaaacgttcctggaactaccggtgctagcctcg |
| | | | aggatatcaagatctggcctcggcggccaagcttggcaatccggtactgttggtaaa |
| | | | gccacc |
| 82 | PL1 | PL- | ggcctaactggccggtaccactagtgacgtcctgagcgacagtatagtgcacagtga |
| | 095 | Twist_ | cattacagatgtttacgacgaattacagatgtttctcatcgattacagatgtttcag |
| | | v18- | ctcaattacagatgtttgctgctgattacagatgtttaccagagattacagatgttt |
| | | HOXA1_ | gacgtctacgtactgatcagcgatgctcatctcgacctgatcggtacaacttctcac |
| | | v10- | ggaggcttctaagtcattacatacgtagtcattactatacgtgtcattacagatgct |
| | | coreFAM111B | gtcattacacgaactgtcattacgtactcagtcattactacgtacgggaaaagttca |
| | | | gctgagagatataaaagagcagtctttccagcacctgcaaatccagagcggcgggca |
| | | | ctgacgggcacttgcaccgtgtggacagactctccggttctgtgagtggtttttctt |
| | | | ttcccgggtcggacctggagttcttagggggatggctgaaccggtgctagcctcgag |
| | | | gatatcaagatctggcctcggcggccaagcttggcaatccggtactgttggtaaagc |
| | | | cacc |
| 83 | PL1 | PL- | ggcctaactggccggtaccactagtgacgtctgtagctgagcgacagtatagtgcac |
| | 096 | HOXC10_ | agtgactgcagcagtcattgtcgtaaattgagtatcgtcgtaaattgacgaacgtcg |
| | | v14- | taaattagcgacagtcgtaaattagtacctgtcgtaaattactctgcgtcgtaaatt |
| | | coreKIF | gacgtctacgtaggcccgccccctttccttacgcggattggtagctgcaggcttccc |
| | | | tatctgattggccgaacgaacgcagcgcgtaatttaaaatattgtatctgtaacaaa |
| | | | gctgcacctcgtgggcggagttgtgctctgcggctgcgaaagtccagcttcggcgac |
| | | | taggtgtgagtaagccagtatcccaggaggagcaagtggcacgtcttcgggtgagtg |
| | | | tgcggctgtgctggagcccgggttaccagctctttaccggtctagcctcgaggatat |
| | | | caagatctggcctcggcggccaagcttggcaatccggtactgttggtaaagccacc |
| 84 | PL1 | PL- | ggcctaactggccggtaccactagtgacgtctacgtactgatcagcgatgctcatct |
| | 097 | HOXA1_ | cgacctgatcggtacaacttctcacggaggcttctaagtcattacatacgtagtcat |
| | | v10- | tactatacgtgtcattacagatgctgtcattacacgaactgtcattacgtactcagt |
| | | coreCST | cattactacgtaagtggtgggggagtgaaaagagagatggagaaagaggggatgggc |
| | | | agaaagaggaggaggagtcaggggcagggcatggaggtgggtggggctgggctgcca |
| | | | aagcaggataaatgcacacctgcctgctggtctgggctccctgcctcgggctctcac |
| | | | cctcctctcctgcagctccagctttgtgctctaccggtgctagcctcgaggatatca |
| | | | agatctggcctcggcggccaagcttggcaatccggtactgttggtaaagccacc |
| 85 | PL1 | PL- | ggcctaactggccggtaccactagtgacgtctacgtactgatcagcgatgctcatct |
| | 098 | HOXA1_ | cgacctgatcggtacaacttctcacggaggcttctaagtcattacatacgtagtcat |
| | | v10- | tactatacgtgtcattacagatgctgtcattacacgaactgtcattacgtactcagt |
| | | coreKIF | cattactacgtaggcccgccccctttccttacgcggattggtagctgcaggcttccc |
| | | | tatctgattggccgaacgaacgcagcgcgtaatttaaaatattgtatctgtaacaaa |
| | | | gctgcacctcgtgggcggagttgtgctctgcggctgcgaaagtccagcttcggcgac |
| | | | taggtgtgagtaagccagtatcccaggaggagcaagtggcacgtcttcgggtgagtg |
| | | | tgcggctgtgctggagcccgggttaccagctctttaccggtgctagcctcgaggata |
| | | | tcaagatctggcctcggcggccaagcttggcaatccggtactgttggtaaagccacc |
| 86 | PL1 | PL- | ggcctaactggccggtaccactagtgacgtctacgtactgatcagcgatgctcatct |
| | 099 | HOXA1_ | cgacctgatcggtacaacttctcacggaggcttctaagtcattacatacgtagtcat |
| | | v10- | tactatacgtgtcattacagatgctgtcattacacgaactgtcattacgtactcagt |
| | | coreCEACAM | cattactacgtaacccacgtgatgctgagaagtactcctgccctaggaagagactca |
| | | | gggcagagggaggaaggacagcagaccagacagtcacagcagccttgacaaaacgtt |
| | | | cctggaactaccggtgctagcctcgaggatatcaagatctggcctcggcggccaagc |
| | | | ttggcaatccggtactgttggtaaagccacc |
| 87 | PL1 | PL- | ggcctaactggccggtaccactagtgacgtctacgtactgatcagcgatgctcatct |
| | 100 | HOXA1_ | cgacctgatcggtacaacttctcacggaggcttctaagtcattacatacgtagtcat |
| | | v10- | tactatacgtgtcattacagatgctgtcattacacgaactgtcattacgtactcagt |
| | | coreAGR2 | cattactacgtacatactgaaaagcatacttttgcaatgttatttttaaaaacaagg |
| | | | aactctttaacccagggaagataatcacttggggaaaggaaggttcgtttctgagtt |
| | | | agcaacaagtaaatgcagcactagtgggtgggattgaggtgtgccctggtgcataaa |
| | | | tagagactcagctgtgctggcacactcagaagcttggaccgcatcctagccgccgac |
| | | | tcacacaaggcaggtgggtgaggaaatccaggtaaggctcctgacagcagctttaga |
| | | | agggtacttgctggagtgaattcgggcctctgattagctagcctcgaggatatcaag |
| | | | atctggcctcggcggccaagcttggcaatccggtactgttggtaaagccacc |
| 88 | PL1 | PL- | ggcctaactggccggtaccactagtgacgtctgtagctgagcgacagtatagtgcac |
| | 101 | HOXC10_ | agtgactgcagcagtcattgtcgtaaattgagtatcgtcgtaaattgacgaacgtcg |
| | | v14- | taaattagcgacagtcgtaaattagtacctgtcgtaaattactctgcgtcgtaaatt |
| | | coreCST | gacgtctacgtaagtggtgggggagtgaaaagagagatggagaaagaggggatgggc |
| | | | agaaagaggaggaggagtcaggggcagggcatggaggtgggtggggctgggctgcca |
| | | | aagcaggataaatgcacacctgcctgctggtctgggctccctgcctcgggctctcac |
| | | | cctcctctcctgcagctccagctttgtgctctaccggtgctagcctcgaggatatca |
| | | | agatctggcctcggcggccaagcttggcaatccggtactgttggtaaagccacc |
| 89 | PL1 | PL- | ggcctaactggccggtaccactagtgacgtctgtagctgagcgacagtatagtgcac |
| | 102 | HOXC10_ | agtgactgcagcagtcattgtcgtaaattgagtatcgtcgtaaattgacgaacgtcg |
| | | v14- | taaattagcgacagtcgtaaattagtacctgtcgtaaattactctgcgtcgtaaatt |
| | | coreFAM111B | gacgtctacgtacgggaaaagttcagctgagagatataaaagagcagtctttccagc |
| | | | acctgcaaatccagagcggcgggcactgacgggcacttgcaccgtgtggacagactc |
| | | | tccggttctgtgagtggtttttcttttcccgggtcggacctggagttcttaggggga |
| | | | tggctgaaccggtgctagcctcgaggatatcaagatctggcctcggcggccaagctt |
| | | | ggcaatccggtactgttggtaaagccacc |
| 90 | PL1 | PL- | ggcctaactggccggtacaactagtgacgtctgtagctgagcgacagtatagtgcac |
| | 103 | HOXC10_ | agtgactgcagcagtcattgtcgtaaattgagtatcgtcgtaaattgacgaacgtcg |
| | | v14- | taaattagcgacagtcgtaaattagtacctgtcgtaaattactctgcgtcgtaaatt |
| | | coreAGR2 | gacgtctacgtacatactgaaaagcatacttttgcaatgttatttttaaaaacaagg |
| | | | aactctttaacccagggaagataatcacttggggaaaggaaggttcgtttctgagtt |
| | | | agcaacaagtaaatgcagcactagtgggtgggattgaggtgtgccctggtgcataaa |
| | | | tagagactcagctgtgctggcacactcagaagcttggaccgcatcctagccgccgac |
| | | | tcacacaaggcaggtgggtgaggaaatccaggtaaggctcctgacagcagctttaga |
| | | | agggtacttgctggagtgaattcgggcctctgattactagcctcgaggatatcaaga |
| | | | tctggcctcggcggccaagcttggcaatccggtactgttggtaaagccacc |
| 91 | PL1 | PL- | ggcctaactggccggtaccactagtgacgtctgtagctgagcgacagtatagtgcac |
| | 104 | HOXC10_ | agtgactgcagcagtcattgtcgtaaattgagtatcgtcgtaaattgacgaacgtcg |
| | | v14- | taaattagcgacagtcgtaaattagtacctgtcgtaaattactctgcgtcgtaaatt |
| | | coreCEACAM | gacgtctacgtaacccacgtgatgctgagaagtactcctgccctaggaagagactca |
| | | | gggcagagggaggaaggacagcagaccagacagtcacagcagccttgacaaaacgtt |
| | | | cctggaactaccggtgctagcctcgaggatatcaagatctggcctcggcggccaagc |
| | | | ttggcaatccggtactgttggtaaagccacc |
| 92 | PL | PL- | ggcctaactggccggtaccactagtgacgtctacgtactgatcagcgatgctcatct |
| | 105 | HOXA1_ | cgacctgatcggtacaacttctcacggaggcttctaagtcattacatacgtagtcat |
| | | v10- | tactatacgtgtcattacagatgctgtcattacacgaactgtcattacgtactcagt |
| | | coreFAM111B | cattactacgtacgggaaaagttcagctgagagatataaaagagcagtctttccagc |
| | | | acctgcaaatccagagcggcgggcactgacgggcacttgcaccgtgtggacagactc |
| | | | tccggttctgtgagtggtttttcttttcccgggtcggacctggagttcttaggggga |
| | | | tggctgaaccggtgctagcctcgaggatatcaagatctggcctcggcggccaagctt |
| | | | ggcaatccggtactgttggtaaagccacc |
| 93 | PL1 | PL- | ggcctaactggccggtaccactagtgacgtctgtagctgagcgacagtatagtgcac |
| | 106 | HOXC10_ | agtgactgcagcagtcattgtcgtaaattgagtatcgtcgtaaattgacgaacgtcg |
| | | v14- | taaattagcgacagtcgtaaattagtacctgtcgtaaattactctgcgtcgtaaatt |
| | | CREB_ | gacgtctacgtaacatcggctatgctgctgctaatgccacgtcaccacatcgacatg |
| | | v6- | ccacgtcaccatcatgccatgccacgtcaccactgcaagatgccacgtcaccacagt |
| | | coreCST | ataatgccacgtcaccaagttactatgccacgtcaccaggtacctacgtaagtggtg |
| | | | ggggagtgaaaagagagatggagaaagaggggatgggcagaaagaggaggaggagtc |
| | | | aggggcagggcatggaggtgggtggggctgggctgccaaagcaggataaatgcacac |
| | | | ctgcctgctggtctgggctccctgcctcgggctctcaccctcctctcctgcagctcc |
| | | | agctttgtgctctaccggtgctagcctcgaggatatcaagatctggcctcggcggcc |
| | | | aagcttggcaatccggtactgttggtaaagccacc |
| 94 | PL1 | PL- | ggcctaactggccggtacactagtgacgtctgtagctgagcgacagtatagtgcaca |
| | 107 | HOXC10_ | gtgactgcagcagtcattgtcgtaaattgagtatcgtcgtaaattgacgaacgtcgt |
| | | v14- | aaattagcgacagtcgtaaattagtacctgtcgtaaattactctgcgtcgtaaattg |
| | | CREB_ | acgtctacgtaacatcggctatgctgctgctaatgccacgtcaccacatcgacatgc |
| | | v6- | cacgtcaccatcatgccatgccacgtcaccactgcaagatgccacgtcaccacagta |
| | | coreKIF | taatgccacgtcaccaagttactatgccacgtcaccaggtacctacgtaggcccgcc |
| | | | ccctttccttacgcggattggtagctgcaggcttccctatctgattggccgaacgaa |
| | | | cgcagcgcgtaatttaaaatattgtatctgtaacaaagctgcacctcgtgggcggag |
| | | | ttgtgctctgcggctgcgaaagtccagcttcggcgactaggtgtgagtaagccagta |
| | | | tcccaggaggagcaagtggcacgtcttcgggtgagtgtgcggctgtgctggagcccg |
| | | | ggttaccagctctttaccggtctagcctcgaggatatcaagatctggcctcggcggc |
| | | | caagcttggcaatccggtactgttggtaaagccacc |
| 95 | PL1 | PL- | ggcctaactggccggtacaactagtgacgtctgtagctgagcgacagtatagtgcac |
| | 108 | HOXC10_ | agtgactgcagcagtcattgtcgtaaattgagtatcgtcgtaaattgacgaacgtcg |
| | | v14- | taaattagcgacagtcgtaaattagtacctgtcgtaaattactctgcgtcgtaaatt |
| | | CREB_ | gacgtctacgtaacatcggctatgctgctgctaatgccacgtcaccacatcgacatg |
| | | v6- | ccacgtcaccatcatgccatgccacgtcaccactgcaagatgccacgtcaccacagt |
| | | coreAGR2 | ataatgccacgtcaccaagttactatgccacgtcaccaggtacctacgtacatactg |
| | | | aaaagcatacttttgcaatgttatttttaaaaacaaggaactctttaacccagggaa |
| | | | gataatcacttggggaaaggaaggttcgtttctgagttagcaacaagtaaatgcagc |
| | | | actagtgggtgggattgaggtgtgccctggtgcataaatagagactcagctgtgctg |
| | | | gcacactcagaagcttggaccgcatcctagccgccgactcacacaaggcaggtgggt |
| | | | gaggaaatccaggtaaggctcctgacagcagctttagaagggtacttgctggagtga |
| | | | attcgggcctctgattactagcctcgaggatatcaagatctggcctcggcggccaag |
| | | | cttggcaatccggtactgttggtaaagccacc |
| 96 | PL1 | PL- | ggcctaactggccggtaccactagtgacgtctgtagctgagcgacagtatagtgcac |
| | 109 | HOXC10_ | agtgactgcagcagtcattgtcgtaaattgagtatcgtcgtaaattgacgaacgtcg |
| | | v14- | taaattagcgacagtcgtaaattagtacctgtcgtaaattactctgcgtcgtaaatt |
| | | CREB_ | gacgtctacgtaacatcggctatgctgctgctaatgccacgtcaccacatcgacatg |
| | | v6- | ccacgtcaccatcatgccatgccacgtcaccactgcaagatgccacgtcaccacagt |
| | | coreCEACAM | ataatgccacgtcaccaagttactatgccacgtcaccaggtacctacgtaacccacg |
| | | | tgatgctgagaagtactcctgccctaggaagagactcagggcagagggaggaaggac |
| | | | agcagaccagacagtcacagcagccttgacaaaacgttcctggaactaccggtgcta |
| | | | gcctcgaggatatcaagatctggcctcggcggccaagcttggcaatccggtactgtt |
| | | | ggtaaagccacc |
| 97 | PL1 | PL- | ggcctaactggccggtaccactagtgacgtctgtagctgagcgacagtatagtgcac |
| | 110 | HOXC10_ | agtgactgcagcagtcattgtcgtaaattgagtatcgtcgtaaattgacgaacgtcg |
| | | v14- | taaattagcgacagtcgtaaattagtacctgtcgtaaattactctgcgtcgtaaatt |
| | | CREB_ | gacgtctacgtaacatcggctatgctgctgctaatgccacgtcaccacatcgacatg |
| | | v6- | ccacgtcaccatcatgccatgccacgtcaccactgcaagatgccacgtcaccacagt |
| | | coreFAM111B | ataatgccacgtcaccaagttactatgccacgtcaccaggtacctacgtacgggaaa |
| | | | agttcagctgagagatataaaagagcagtctttccagcacctgcaaatccagagcgg |
| | | | cgggcactgacgggcacttgcaccgtgtggacagactctccggttctgtgagtggtt |
| | | | tttcttttcccgggtcggacctggagttcttagggggatggctgaaccggtgctagc |
| | | | ctcgaggatatcaagatctggcctcggcggccaagcttggcaatccggtactgttgg |
| | | | taaagccacc |
| 98 | PL1 | PL- | ggcctaactggccggtaccactagtgacgtctacgtaacatcggctatgctgctgct |
| | 111 | CREB_ | aatgccacgtcaccacatcgacatgccacgtcaccatcatgccatgccacgtcacca |
| | | v6- | ctgcaagatgccacgtcaccacagtataatgccacgtcaccaagttactatgccacg |
| | | coreCST | tcaccaggtacctacgtaagtggtgggggagtgaaaagagagatggagaaagagggg |
| | | | atgggcagaaagaggaggaggagtcaggggcagggcatggaggtgggtggggctggg |
| | | | ctgccaaagcaggataaatgcacacctgcctgctggtctgggctccctgcctcgggc |
| | | | tctcaccctcctctcctgcagctccagctttgtgctctaccggtgctagcctcgagg |
| | | | atatcaagatctggcctcggcggccaagcttggcaatccggtactgttggtaaagcc |
| | | | acc |
| 99 | PL1 | PL- | ggcctaactggccggtacaactagtgacgtctacgtaacatcggctatgctgctgct |
| | 112 | CREB_ | aatgccacgtcaccacatcgacatgccacgtcaccatcatgccatgccacgtcacca |
| | | v6- | ctgcaagatgccacgtcaccacagtataatgccacgtcaccaagttactatgccacg |
| | | coreAGR2 | tcaccaggtacctacgtacatactgaaaagcatacttttgcaatgttatttttaaaa |
| | | | acaaggaactctttaacccagggaagataatcacttggggaaaggaaggttcgtttc |
| | | | tgagttagcaacaagtaaatgcagcactagtgggtgggattgaggtgtgccctggtg |
| | | | cataaatagagactcagctgtgctggcacactcagaagcttggaccgcatcctagcc |
| | | | gccgactcacacaaggcaggtgggtgaggaaatccaggtaaggctcctgacagcagc |
| | | | tttagaagggtacttgctggagtgaattcgggcctctgattactagcctcgaggata |
| | | | tcaagatctggcctcggcggccaagcttggcaatccggtactgttggtaaagccacc |
| 100 | PL1 | PL- | ggcctaactggccggtaccactagtgacgtctacgtaacatcggctatgctgctgct |
| | 113 | CREB_ | aatgccacgtcaccacatcgacatgccacgtcaccatcatgccatgccacgtcacca |
| | | v6- | ctgcaagatgccacgtcaccacagtataatgccacgtcaccaagttactatgccacg |
| | | coreKIF | tcaccaggtacctacgtaggcccgccccctttccttacgcggattggtagctgcagg |
| | | | cttccctatctgattggccgaacgaacgcagcgcgtaatttaaaatattgtatctgt |
| | | | aacaaagctgcacctcgtgggcggagttgtgctctgcggctgcgaaagtccagcttc |
| | | | ggcgactaggtgtgagtaagccagtatcccaggaggagcaagtggcacgtcttcggg |
| | | | tgagtgtgcggctgtgctggagcccgggttaccagctctttaccggtctagcctcga |
| | | | ggatatcaagatctggcctcggcggccaagcttggcaatccggtactgttggtaaag |
| | | | ccacc |
| 101 | PL1 | PL- | ggcctaactggccggtaccactagtgacgtctacgtaacatcggctatgctgctgct |
| | 114 | CREB_ | aatgccacgtcaccacatcgacatgccacgtcaccatcatgccatgccacgtcacca |
| | | v6- | ctgcaagatgccacgtcaccacagtataatgccacgtcaccaagttactatgccacg |
| | | coreCEACAM | tcaccaggtacctacgtaacccacgtgatgctgagaagtactcctgccctaggaaga |
| | | | gactcagggcagagggaggaaggacagcagaccagacagtcacagcagccttgacaa |
| | | | aacgttcctggaactaccggtgctagcctcgaggatatcaagatctggcctcggcgg |
| | | | ccaagcttggcaatccggtactgttggtaaagccacc |
| 102 | PL1 | PL- | ggcctaactggccggtaccactagtgacgtctacgtaacatcggctatgctgctgct |
| | 115 | CREB_ | aatgccacgtcaccacatcgacatgccacgtcaccatcatgccatgccacgtcacca |
| | | v6- | ctgcaagatgccacgtcaccacagtataatgccacgtcaccaagttactatgccacg |
| | | coreFAM111B | tcaccaggtacctacgtacgggaaaagttcagctgagagatataaaagagcagtctt |
| | | | tccagcacctgcaaatccagagcggcgggcactgacgggcacttgcaccgtgtggac |
| | | | agactctccggttctgtgagtggtttttcttttcccgggtcggacctggagttctta |
| | | | gggggatggctgaaccggtgctagcctcgaggatatcaagatctggcctcggcggcc |
| | | | aagcttggcaatccggtactgttggtaaagccacc |
| 103 | PL1 | HES6_ | GAATTCaagaCtgcaagCGAGCGACAGTATAGTGCACAGTGACTGCAGCAGTCATTA |
| | 144 | v11- | TACGTCGCCTAAATCGAGATGCTGTAGGCACGTGTATCTGGCACGTGTACTCGGCAC |
| | | coreBIRC5 | GTGTACTAGGCACGTGTAAGAGGCACGTGTACGCGGCACGTGTAGGTACCTGCGCTC |
| | | | CCGACATGCCCCGCGGCGCGCCATTAACCGCCAGATTTGAGTCGCGGGACCCGTTGG |
| | | | CAGAGGTGGGCTAGCCTCGAGGATATCAAGATCTGGCCTCGGCGGCCAAGCTTGGCA |
| | | | ATCCGGTACTGTTGGTAAAGCCACCATGGAAG |
| 104 | PL1 | HES6_ | GAATTCaagaCtgcaagCGAGCGACAGTATAGTGCACAGTGACTGCAGCAGTCATTA |
| | 145 | v11- | TACGTCGCCTAAATCGAGATGCTGTAGGCACGTGTATCTGGCACGTGTACTCGGCAC |
| | | TATA- | GTGTACTAGGCACGTGTAAGAGGCACGTGTACGCGGCACGTGTAGGTACCTATAAAA |
| | | TSS | GGCCAGCAGCAGCCTGACCACATCTCATCCGCTAGCCTCGAGGATATCAAGATCTGG |
| | | | CCTCGGCGGCCAAGCTTGGCAATCCGGTACTGTTGGTAAAGCCACC |
| 105 | PL1 | NPAS2_ | GAATTCaagaCtgcaagCCTGAGCGACAGTATAGTGCACAGTGACTGCAGCAGTCAT |
| | 146 | v11- | TATACGTCGCCTAAATCGAGATGCTGGACACGTGTCCGAGACACGTGTCTGTGACAC |
| | | coreBIRC5 | GTGTCCGGGACACGTGTCGCAGACACGTGTCGTGGACACGTGTCGGTACCTGCGCTC |
| | | | CCGACATGCCCCGCGGCGCGCCATTAACCGCCAGATTTGAGTCGCGGGACCCGTTGG |
| | | | CAGAGGTGGGCTAGCCTCGAGGATATCAAGATCTGGCCTCGGCGGCCAAGCTTGGCA |
| | | | ATCCGGTACTGTTGGTAAAGCCACC |
| 106 | PL1 | NPAS2_ | GAATTCaagaCtgcaagCCTGAGCGACAGTATAGTGCACAGTGACTGCAGCAGTCAT |
| | 147 | v11- | TATACGTCGCCTAAATCGAGATGCTGGACACGTGTCCGAGACACGTGTCTGTGACAC |
| | | TATA- | GTGTCCGGGACACGTGTCGCAGACACGTGTCGTGGACACGTGTCGGTACCTATAAAA |
| | | TSS | GGCCAGCAGCAGCCTGACCACATCTCATCCGCTAGCCTCGAGGATATCAAGATCTGG |
| | | | CCTCGGCGGCCAAGCTTGGCAATCCGGTACTGTTGGTAAAGCCACC |
| 107 | PL1 | pGL4.10- | ggcctaactggccggtaccactagtatcgatccttcatagggcagggaggggtgggc |
| | 15 | FAM83A-43 | acttgggtgtgaccaaggagaggaggcgcgcctggtcaacagctctccctggcccgt |
| | | | gtccagctccctcctcacacagagaggggggcgcatctcagggatggcatctttccc |
| | | | ccccacagggaaattcttatctttgaaacagcatgggaatcgaggcacccaggaggg |
| | | | gagcagaggcaggcaggcctccttcaggcccatcctccagctgggctggtggtgcca |
| | | | gggaggctccctgcttggtaacaaaggcctgagggagagttgcgaaacccagcagga |
| | | | aagccggctcaccttcgcctccccctgcggctgggaggagaggaaatatcccatggc |
| | | | tgactgtgccaaggaggtgtctgagccagccctcccggcccgagggcagggcaggtg |
| | | | gccctgagagataagccaatcccgcagctgcagatgaggagttctgagaagcattgc |
| | | | tcaggacagcggtaaatcacttcttggaggtgccctgcacgccggtcctgggagcag |
| | | | gcggcctcccgggggtgcgggagccccactcctccgtggtgtgttccatttgcttcc |
| | | | cacatctggaggagctgacgtgccagcctcccccagcaccacccagggacgggaggc |
| | | | aaccggtgctagcctcgaggatatcaagatctggcctcggcggccaagcttggcaat |
| | | | ccggtactgttggtaaagccacc |
| 108 | PL1 | PL- | ggcctaactggccggtaccgacgtctacctgatcaaacatgcccggacatgtcgtaa |
| | 156 | TP53_v5- | gacataaacatgcccggacatgtcctcgcaatctaacatgcccggacatgtcctcgc |
| | | TATA- | aatctaacatgcccggacatgtctgcaagctacaacatgcccggacatgtctacgta |
| | | TSS | gctagctataaaaggccagcagcagcctgaccacatctcatcctcctcgaggatatc |
| | | FLUC | aagatctggcctcggcggccaagcttggcaatccggtactgttggtaaagccacc |
| 109 | PL1 | PL- | ggcctaactggccggtaccgacgtccctgatcggtacaacttctcacaacatgcctg |
| | 157 | TP53_ | ggcatgtcgctatgcaacatgcctgggcatgtcagatgcaaacatgcctgggcatgt |
| | | v22-TATA- | cctgctataacatgcctgggcatgtcctgctataacatgcctgggcatgtctacgta |
| | | TSS | gctagctataaaaggccagcagcagcctgaccacatctcatcctcctcgaggatatc |
| | | FLUC | aagatctggcctcggcggccaagcttggcaatccggtactgttggtaaagccacc |
| 110 | PL1 | PL-TP53 | ggcctaactggccggtaccgacgtctcgggcaagcgctcccgacatgcccgggcaag |
| | 158 | SURV_ | cgctcccgacatgcccgggcaagcgctcccgacatgcccgggcaagcgctcccgaca |
| | | v3-TATA- | tgcccgggcaagcgctcccgacatgcccgggcaagcgctcccgacatgccctacgta |
| | | TSS | gctagctataaaaggccagcagcagcctgaccacatctcatcctcctcgaggatatc |
| | | FLUC | aagatctggcctcggcggccaagcttggcaatccggtactgttggtaaagccacc |
| 111 | PL1 | PL- | ggcctaactggccggtaccttttgataaaaatcattaggtacggccgcggtgccagg |
| | 159 | TCF7_v2- | gcgtgcccttgggctccccgggcgcgaaactagtgacgtcctgagcgacagtatagt |
| | | FOS- | gcacagtgactgcagcagtcattcctttgatgtacgcaactcctttgatgtctatgc |
| | | coreBIRC5 | gtcctttgatgttaaggattcctttgatgtaggtacatcctttgatgtccgtaaatc |
| | | | ctttgatgtgacgtctacgtaggtgactcatgggtgactcatgtacgtaacgcgtcc |
| | | | cgacatgccccgcggcgcgccattaaccgccagatttgagtcgcgggacccgttggc |
| | | | agaggtgggaattcaccggtgctagcctcgaggatatcaagatctggcctcggcggc |
| | | | caagcttggcaatccggtactgttggtaaagccacc |
| 112 | PL1 | PL-FOS- | ggcctaactggccggtaccttttgataaaaatcattaggtacggccgcggtgccagg |
| | 160 | TCF_v2- | gcgtgcccttgggctccccgggcgcgaaactagtgacgtcggtgactcatgggtgac |
| | | coreBIRC5 | tcatgacgtctacgtactgagcgacagtatagtgcacagtgactgcagcagtcattc |
| | | | ctttgatgtacgcaactcctttgatgtctatgcgtcctttgatgttaaggattcctt |
| | | | tgatgtaggtacatcctttgatgtccgtaaatcctttgatgttacgtaacgcgtccc |
| | | | gacatgccccgcggcgcgccattaaccgccagatttgagtcgcgggacccgttggca |
| | | | gaggtgggaattcaccggtgctagcctcgaggatatcaagatctggcctcggcggcc |
| | | | aagcttggcaatccggtactgttggtaaagccacc |
| 113 | PL1 | PL- | ggcctaactggccggtaccaactagtgacgtcctgagcgacagtatagtgcacagtg |
| | 161 | TCF7_ | actgcagcagtcattcctttgatgtacgcaactcctttgatgtctatgcgtcctttg |
| | | v2-FOS- | atgttaaggattcctttgatgtaggtacatcctttgatgtccgtaaatcctttgatg |
| | | coreAGR2 | tgacgtctacgtaggtgactcatgggtgactcatgtacgtacatactgaaaagcata |
| | | | cttttgcaatgttatttttaaaaacaaggaactctttaacccagggaagataatcac |
| | | | ttggggaaaggaaggttcgtttctgagttagcaacaagtaaatgcagcactagtggg |
| | | | tgggattgaggtgtgccctggtgcataaatagagactcagctgtgctggcacactca |
| | | | gaagcttggaccgcatcctagccgccgactcacacaaggcaggtgggtgaggaaatc |
| | | | caggtaaggctcctgacagcagctttagaagggtacttgctggagtgaattcgggcc |
| | | | tctgattagctagcctcgaggatatcaagatctggcctcggcggccaagcttggcaa |
| | | | tccggtactgttggtaaagccacc |
| 114 | PL1 | PL-FOS- | ggcctaactggccggtaccaactagtgacgtcggtgactcatgggtgactcatggac |
| | 162 | TCF7_ | gtctacgtactgagcgacagtatagtgcacagtgactgcagcagtcattcctttgat |
| | | v2- | gtacgcaactcctttgatgtctatgcgtcctttgatgttaaggattcctttgatgta |
| | | coreAGR2 | ggtacatcctttgatgtccgtaaatcctttgatgttacgtacatactgaaaagcata |
| | | | cttttgcaatgttatttttaaaaacaaggaactctttaacccagggaagataatcac |
| | | | ttggggaaaggaaggttcgtttctgagttagcaacaagtaaatgcagcactagtggg |
| | | | tgggattgaggtgtgccctggtgcataaatagagactcagctgtgctggcacactca |
| | | | gaagcttggaccgcatcctagccgccgactcacacaaggcaggtgggtgaggaaatc |
| | | | caggtaaggctcctgacagcagctttagaagggtacttgctggagtgaattcgggcc |
| | | | tctgattagctagcctcgaggatatcaagatctggcctcggcggccaagcttggcaa |
| | | | tccggtactgttggtaaagccacc |
| 115 | PL1 | PL- | ggcctaactggccggtaccaactagtgacgtcctgagcgacagtatagtgcacagtg |
| | 163 | TCF7_ | actgcagcagtcattcctttgatgtacgcaactcctttgatgtctatgcgtcctttg |
| | | v2- | atgttaaggattcctttgatgtaggtacatcctttgatgtccgtaaatcctttgatg |
| | | coreAGR2 | tgacgtctacgtacatactgaaaagcatacttttgcaatgttatttttaaaaacaag |
| | | | gaactctttaacccagggaagataatcacttggggaaaggaaggttcgtttctgagt |
| | | | tagcaacaagtaaatgcagcactagtgggtgggattgaggtgtgccctggtgcataa |
| | | | atagagactcagctgtgctggcacactcagaagcttggaccgcatcctagccgccga |
| | | | ctcacacaaggcaggtgggtgaggaaatccaggtaaggctcctgacagcagctttag |
| | | | aagggtacttgctggagtgaattcgggcctctgattagctagcctcgaggatatcaa |
| | | | gatctggcctcggcggccaagcttggcaatccggtactgttggtaaagccacc |
| 116 | PL1 | PL- | CAACTAGTGACGTCCTGAGCGACAGTATAGTGCACAGTGACTGCAGCAGTCATTCCT |
| | 164 | TCF7_ | TTGATGTACGCAACTCCTTTGATGTCTATGCGTCCTTTGATGTTAAGGATTCCTTTG |
| | | v2-FOS- | ATGTAGGTACATCCTTTGATGTCCGTAAATCCTTTGATGTGACGTCTACGTAGGTGA |
| | | coreCEACAM5 | CTCATGGGTGACTCATGTACGTAACCCACGTGATGCTGAGAAGTACTCCTGCCCTAG |
| | | | GAAGAGACTCAGGGCAGAGGGAGGAAGGACAGCAGACCAGACAGTCACAGCAGCCTT |
| | | | GACAAAACGTTCCTGGAACTACCGGT |
| 117 | PL1 | PL- | ggcctaactggccggtaccaactagtgacgtcctgagcgacagtatagtgcacagtg |
| | 165 | TCF7_v2- | actgcagcagtcattcctttgatgtacgcaactcctttgatgtctatgcgtcctttg |
| | | coreCEACAM5 | atgttaaggattcctttgatgtaggtacatcctttgatgtccgtaaatcctttgatg |
| | | | tgacgtctacgtaacccacgtgatgctgagaagtactcctgccctaggaagagactc |
| | | | agggcagagggaggaaggacagcagaccagacagtcacagcagccttgacaaaacgt |
| | | | tcctggaactaccggtgctagcctcgaggatatcaagatctggcctcggcggccaag |
| | | | cttggcaatccggtactgttggtaaagccacc |
| 118 | PL1 | PL- | AACTAGTGACGTCCTGAGCGACAGTATAGTGCACAGTGACTGCAGCAGTCATTCCTT |
| | 166 | TCF7_v2- | TGATGTACGCAACTCCTTTGATGTCTATGCGTCCTTTGATGTTAAGGATTCCTTTGA |
| | | coreFAM111B | TGTAGGTACATCCTTTGATGTCCGTAAATCCTTTGATGTGACGTCTACGTATACGTA |
| | | | CGGGAAAAGTTCAGCTGAGAGATATAAAAGAGCAGTCTTTCCAGCACCTGCAAATCC |
| | | | AGAGCGGCGGGCACTGACGGGCACTTGCACCGTGTGGACAGACTCTCCGGTTCTGTG |
| | | | AGTGGTTTTTCTTTTCCCGGGTCGGACCTGGAGTTCTTAGGGGGATGGCTGaaccgg |
| | | | t |
| 119 | PL1 | PL- | CTAGTGACGTCCTGAGCGACAGTATAGTGCACAGTGACTGCAGCAGTCATTCCTTTG |
| | 167 | TCF7_v2- | ATGTACGCAACTCCTTTGATGTCTATGCGTCCTTTGATGTTAAGGATTCCTTTGATG |
| | | coreCST | TAGGTACATCCTTTGATGTCCGTAAATCCTTTGATGTGACGTCTACGTATACGTAAG |
| | | | TGGTGGGGGAGTGAAAAGAGAGATGGAGAAAGAGGGGATGGGCAGAAAGAGGAGGAG |
| | | | GAGTCAGGGGCAGGGCATGGAGGTGGGTGGGGCTGGGCTGCCAAAGCAGGATAAATG |
| | | | CACACCTGCCTGCTGGTCTGGGCTCCCTGCCTCGGGCTCTCACCCTCCTCTCCTGCA |
| | | | GCTCCAGCTTTGTGCTCTa |
| 120 | PL1 | PL- | CTAGTGACGTCCTGAGCGACAGTATAGTGCACAGTGACTGCAGCAGTCATTCCTTTG |
| | 168 | TCF7_v2- | ATGTACGCAACTCCTTTGATGTCTATGCGTCCTTTGATGTTAAGGATTCCTTTGATG |
| | | coreKIF20A | TAGGTACATCCTTTGATGTCCGTAAATCCTTTGATGTGACGTCTACGTATACGTAGG |
| | | | CCCGCCCCCTTTCCTTACGCGGATTGGTAGCTGCAGGCTTCCCTATCTGATTGGCCG |
| | | | AACGAACGCAGCGCGTAATTTAAAATATTGTATCTGTAACAAAGCTGCACCTCGTGG |
| | | | GCGGAGTTGTGCTCTGCGGCTGCGAAAGTCCAGCTTCGGCGACTAGGTGTGAGTAAG |
| | | | CCAGTATCCCAGGAGGAGCAAGTGGCACGTCTTCGGGTGAGTGTGCGGCTGTGCTGG |
| | | | AGCCCGGGTTACCAGCTCTTTA |
| 121 | PL1 | pGL4.10- | ggcctaactggccggtaccaccatggggaaggtggggtgatcacaggacagtcagcc |
| | 17 | CEACAM5 | tcgcagaggacagagaccacccaggactgtcagggagaacatggacaggccctgagc |
| | | | cgcagctcagccaacagacacggagagggagggtccccctggagccttccccaagga |
| | | | cagcagagcccagagtcacccacctccctccaccacagtcctctctttccaggacac |
| | | | acaagacacctccccctccacatgcaggatctggggactcctgagacctctgggcct |
| | | | gggtctccatccctgggtcagtggcggggttggtggtactggagacagagggctggt |
| | | | ccctccccagccaccacccagtgagcctttttctagcccccagagccacctctgtca |
| | | | ccttcctgttgggcatcatcccaccttcccagagccctggagagcatggggagaccc |
| | | | gggaccctgctgggtttctctgtcacaaaggaaaataatccccctggtgtgacagac |
| | | | ccaaggacagaacacagcagaggtcagcactggggaagacaggttgtcctcccaggg |
| | | | gatgggggtccatccaccttgccgaaaagatttgtctgaggaactgaaaatagaagg |
| | | | gaaaaaagaggagggacaaaagaggcagaaatgagaggggaggggacagaggacacc |
| | | | tgaataaagaccacacccatgacccacgtgatgctgagaagtactcctgccctagga |
| | | | agagactcagggcagagggaggaaggacagcagaccagacagtcacagcagccttga |
| | | | caaaacgttcctggaactaccggtgctagcctcgaggatatcaagatctggcctcgg |
| | | | cggccaagcttggcaatccggtactgttggtaaagccacc |
| 122 | PL1 | PL- | ggcctaactggccggtaccttttgataaaaatcattaggtacggccgcggtgccagg |
| | 183 | TP53_ | gcgtgcccttgggctccccgggcgcgaaactagtgacgtctacctgatcaaacatgc |
| | | v5- | ccggacatgtcgtaagacataaacatgcccggacatgtcctcgcaatctaacatgcc |
| | | coreBIRC5 | cggacatgtcctcgcaatctaacatgcccggacatgtctgcaagctacaacatgccc |
| | | | ggacatgtctacgtaacgcgtcccgacatgccccgcggcgcgccattaaccgccaga |
| | | | tttgagtcgcgggacccgttggcagaggtgggaattcaccggtgctagcctcgagga |
| | | | tatcaagatctggcctcggcggccaagcttggcaatccggtactgttggtaaagcca |
| | | | CC |
| 123 | PL1 | PL- | ggcctaactggccggtaccaactagtgacgtctacctgatcaaacatgcccggacat |
| | 184 | TP53_ | gtcgtaagacataaacatgcccggacatgtcctcgcaatctaacatgcccggacatg |
| | | v5- | tcctcgcaatctaacatgcccggacatgtctgcaagctacaacatgcccggacatgt |
| | | coreAGR2 | ctacgtacatactgaaaagcatacttttgcaatgttatttttaaaaacaaggaactc |
| | | | tttaacccagggaagataatcacttggggaaaggaaggttcgtttctgagttagcaa |
| | | | caagtaaatgcagcactagtgggtgggattgaggtgtgccctggtgcataaatagag |
| | | | actcagctgtgctggcacactcagaagcttggaccgcatcctagccgccgactcaca |
| | | | caaggcaggtgggtgaggaaatccaggtaaggctcctgacagcagctttagaagggt |
| | | | acttgctggagtgaattcgggcctctgattagctagcctcgaggatatcaagatctg |
| | | | gcctcggcggccaagcttggcaatccggtactgttggtaaagccacc |
| 124 | PL1 | PL- | ggcctaactggccggtaccaactagtgacgtctacctgatcaaacatgcccggacat |
| | 185 | TP53_ | gtcgtaagacataaacatgcccggacatgtcctcgcaatctaacatgcccggacatg |
| | | v5- | tcctcgcaatctaacatgcccggacatgtctgcaagctacaacatgcccggacatgt |
| | | coreFAM111B | ctacgtacgggaaaagttcagctgagagatataaaagagcagtctttccagcacctg |
| | | | caaatccagagcggcgggcactgacgggcacttgcaccgtgtggacagactctccgg |
| | | | ttctgtgagtggtttttcttttcccgggtcggacctggagttcttagggggatggct |
| | | | gaaccggtgctagcctcgaggatatcaagatctggcctcggcggccaagcttggcaa |
| | | | tccggtactgttggtaaagccacc |
| 125 | PL1 | PL- | ggcctaactggccggtaccaactagtgacgtctacctgatcaaacatgcccggacat |
| | 186 | TP53_ | gtcgtaagacataaacatgcccggacatgtcctcgcaatctaacatgcccggacatg |
| | | v5- | tcctcgcaatctaacatgcccggacatgtctgcaagctacaacatgcccggacatgt |
| | | coreCST | ctacccgttcgacaagcccggacatgctaagacataaacatgcccggacatgtcctc |
| | | | gcaatctaaccatgcccggacatgtcctcgcaatctaacatgcccggacatgtctgc |
| | | | aagctacaacatgcccggacatgtctacgtaagtggtgggggagtgaaaagagagat |
| | | | ggagaaagaggggatgggcagaaagaggaggaggagtcaggggcagggcatggaggt |
| | | | gggtggggctgggctgccaaagcaggataaatgcacacctgcctgctggtctgggct |
| | | | ccctgcctcgggctctcaccctcctctcctgcagctccagctttgtgctctaccggt |
| | | | gctagcctcgaggatatcaagatctggcctcggcggccaagcttggcaatccggtac |
| | | | tgttggtaaagccacc |
| 126 | PL1 | PL- | ggcctaactggccggtaccttttgataaaaatcattaggtacggccgcggtgccagg |
| | 187 | TCF7_v2- | gcgtgcccttgggctccccgggcgcgaaactagtgacgtcctgagcgacagtatagt |
| | | TP53_v5- | gcacagtgactgcagcagtcattcctttgatgtacgcaactcctttgatgtctatgc |
| | | coreBIRC5 | gtcctttgatgttaaggattcctttgatgtaggtacatcctttgatgtccgtaaatc |
| | | | ctttgatgtgacgtctacgtatctacctgatcaaacatgcccggacatgtcgtaaga |
| | | | cataaacatgcccggacatgtcctcgcaatctaacatgcccggacatgtcctcgcaa |
| | | | tctaacatgcccggacatgtctgcaagctacaacatgcccggacatgtctacgtaac |
| | | | gcgtcccgacatgccccgcggcgcgccattaaccgccagatttgagtcgcgggaccc |
| | | | gttggcagaggtgggaattcaccggtgctagcctcgaggatatcaagatctggcctc |
| | | | ggcggccaagcttggcaatccggtactgttggtaaagccacc |
| 127 | PL1 | PL- | ggcctaactggccggtaccaactagtgacgtcctgagcgacagtatagtgcacagtg |
| | 188 | TCF7_v2- | actgcagcagtcattcctttgatgtacgcaactcctttgatgtctatgcgtcctttg |
| | | TP53_v5- | atgttaaggattcctttgatgtaggtacatcctttgatgtccgtaaatcctttgatg |
| | | coreAGR2 | tgacgtctacgtatctacctgatcaaacatgcccggacatgtcgtaagacataaaca |
| | | | tgcccggacatgtcctcgcaatctaacatgcccggacatgtcctcgcaatctaacat |
| | | | gcccggacatgtctgcaagctacaacatgcccggacatgtctacaatatacgtatct |
| | | | acctgatcaaacatgcccggacatgtcgtaagacataaacatgcccggacatgtcct |
| | | | cgcaatctaacatgcccggacatgtcctcgcaatctaacatgcccggacatgtctgc |
| | | | aagctacaacatgcccggacatgtctacgtacatactgaaaagcatacttttgcaat |
| | | | gttatttttaaaaacaaggaactctttaacccagggaagataatcacttggggaaag |
| | | | gaaggttcgtttctgagttagcaacaagtaaatgcagcactagtgggtgggattgag |
| | | | gtgtgccctggtgcataaatagagactcagctgtgctggcacactcagaagcttgga |
| | | | ccgcatcctagccgccgactcacacaaggcaggtgggtgaggaaatccaggtaaggc |
| | | | tcctgacagcagctttagaagggtacttgctggagtgaattcgggcctctgattagc |
| | | | tagcctcgaggatatcaagatctggcctcggcggccaagcttggcaatccggtactg |
| | | | ttggtaaagccacc |
| 130 | PL1 | pGL4.10- | ggcctaactggccggtaccactagtaagcctcaagatttcctttaggctcttaggta |
| | 21 | KIF20A | agaaatgtctaaggttcaaggaaaaaggttaagttggaagaatcccaggcaaaataa |
| | | | gtgcgaatccacgacagttggtaacccggacccacattagaactcagaggtcaagca |
| | | | gaagcgaacgactggaattccagtcaggcccgccccctttccttacgcggattggta |
| | | | gctgcaggcttccctatctgattggccgaacgaacgcagcgcgtaatttaaaatatt |
| | | | gtatctgtaacaaagctgcacctcgtgggcggagttgtgctctgcggctgcgaaagt |
| | | | ccagcttcggcgactaggtgtgagtaagccagtatcccaggaggagcaagtggcacg |
| | | | tcttcgggtgagtgtgcggctgtgctggagcccgggttaccagctcttaccggtgct |
| | | | agcctcgaggatatcaagatctggcctcggcggccaagcttggcaatccggtactgt |
| | | | tggtaaagccacc |
| 145 | PL1 | PL- | ggcctaactggccggtaccactagtggggcggggtgatgacacagcaattcgggact |
| | 236 | HIGH- | ttccacgcttgcgtgagaagagaccggaagtgaatgacacagcaattcgcttgcgtg |
| | | coreFAM111B- | agaagctgggactttcctaggggcggggttgggactttccacatgacacagcaatac |
| | | FLUC-HA | actagtaacatttctctggcctaactggccggtaccgggaaaagttcagctgagaga |
| | | | tataaaagagcagtctttccagcacctgcaaatccagagcggcgggcactgacgggc |
| | | | acttgcaccgtgtggacagactctccggttctgtgagtggtttttcttttcccgggt |
| | | | cggacctggagttcttagggggatggctgaagaattcaccggtcgacgctagc |
| 147 | PL1 | PL- | ggcctaactggccggtaccactagtgtcatctctttgaatattctgtagtttgagga |
| | 238 | AFP3- | gaatatttgttatattgcacaataaaataagtttgcaagttttttttttctgcccca |
| | | FLUC- | aagagctctgtgtccttgaacataaaatacaaataaccgctatgctgttaattatta |
| | | HA | acaaatgtcccattttcaacctaaggaaataccataaagtaacagatataccaacaa |
| | | | aaggttaataattaacaggcattgcctgaaaagagtataaaaggctttcagcatgat |
| | | | tttccatattgtgcttccaccactgccaataacaaaccggtgaattcaccggtcgac |
| | | | gctagc |
| 148 | PL1 | FOSL1- | GAATTCACTAGTGACAGTATAGTGCACAGTGACTGCAGCAGGGTGACTCATGATGCC |
| | 239 | v1- | ACGTCACCAGGTGACTCATGATGCCACGTCACCAGGTGACTCATGATGCCACGTCAC |
| | | CREB3L1- | CAGGTGACTCATGATGCCACGTCACCAGGTGACTCATGGGTACCTATAAAAGGCCAG |
| | | v6- | CAGCAGCCTGACCACATCTCATCCA |
| | | 1x1_v1 | |
| 149 | PL1 | FOSL1- | GAATTCACTAGTAGTATAGTGCACAGTGACTGCAGCAGGGTGACTCATGATGATGCC |
| | 240 | v1- | ACGTCACCAATGCCACGTCACCAGGTGACTCATGGGTGACTCATGATGCCACGTCAC |
| | | CREB3L1- | CAATGCCACGTCACCAGGTGACTCATGGGTGACTCATGGGTACCTATAAAAGGCCAG |
| | | v6- | CAGCAGCCTGACCACATCTCATCCA |
| | | 2x2_v1 | |
| 150 | PL1 | FOXO1:: | GAATTCACTAGTCTCAAGTATAAGGTAAGACATAGTTACTGCGACATCGGCTAGTAA |
| | 241 | ELK3_ | ACCGGAAGTGTCTGTAAACCGGAAGTGATCGTAAACCGGAAGTGAGCGTAAACCGGA |
| | | v6 | AGTGCTAGTAAACCGGAAGTGGAAGTAAACCGGAAGTGGGTACCTATAAAAGGCCAG |
| | | | CAGCAGCCTGACCACATCTCATCCA |
| 151 | PL1 | MTF1_ | GAATTCACTAGTGTACTCAAGTATAAGGTAAGATTTGCACACGGTACGTACTCATTT |
| | 242 | v9 | GCACACGGTACATGCGAGTTTGCACACGGTACAGCTCAGTTTGCACACGGTACGTCA |
| | | | GCTTTTGCACACGGTACATCAGAATTTGCACACGGTACGGTACCTATAAAAGGCCAG |
| | | | CAGCAGCCTGACCACATCTCATCCACCGGTG |
| 152 | PL1 | NFE2L2_ | GAATTCACTAGTTAATTGCTGAGTCATTGCTGCTATGTAATTGCTGAGTCATATGCC |
| | 243 | v14 | TATCCTAATTGCTGAGTCATAATCGAGATGTAATTGCTGAGTCATGTCCGACGCATA |
| | | | ATTGCTGAGTCATTCTAACTCGCTAATTGCTGAGTCATGGTACCTATAAAAGGCCAG |
| | | | CAGCAGCCTGACCACATCTCATCCA |
| 153 | PL1 | NFKB1_ | GAATTCACTAGTGCTGAGCGACAGTATAGTGCACAGTGACTGCAGCAGTCATTATAC |
| | 244 | v3 | GTAGGGGAATCCCCTCGAAGGGGAATCCCCTTTAAGGGGAATCCCCTCGCAGGGGAA |
| | | | TCCCCTCTCAGGGGAATCCCCTAACAGGGGAATCCCCTGGTACCTATAAAAGGCCAG |
| | | | CAGCAGCCTGACCACATCTCATCCA |
| 154 | PL1 | TP53-v5- | GAATTCACTAGTGCATCCTTTGATGTTACCTGATCAAACATGCCCGGACATGTCGTA |
| | 245 | TCF7- | AGACATATCCTTTGATGTCTCGCAATCTAACATGCCCGGACATGTCCTCGCAATCTT |
| | | v2- | CCTTTGATGTTGCAAGCTACAACATGCCCGGACATGTCGGTACCTATAAAAGGCCAG |
| | | 1x1_v1 | CAGCAGCCTGACCACATCTCATCCA |
| 155 | PL1 | XBP1_ | GAATTCACTAGTGCACCATTAGTACTTGATCAGTATGCCACGTCATCACTACTCTAT |
| | 246 | v19 | GCCACGTCATCTCCTAGATATGCCACGTCATCGTAAGACTATGCCACGTCATCTACA |
| | | | GCTTATGCCACGTCATCACGTACTTATGCCACGTCATCGGTACCTATAAAAGGCCAG |
| | | | CAGCAGCCTGACCACATCTCATCCA |
| 156 | PL5 | Cancript- | ggcctaactggccggtaccactagtgtccccacccacacattcctgtccccacccac |
| | 50 | coreBIRC5- | acattcctgtccccacccacacattcctgtccccacccacacattcctgtccccacc |
| | | FLUC | cacacattcctgtccccacccacacattcctgtgcgctcccgacatgccccgcggcg |
| | | | cgccattaaccgccagatttgagtcgcgggacccgttggcagaggtgggctagcctc |
| | | | gaggatatcaagatctggcctcggcggccaagcttggcaatccggtactgttggtaa |
| | | | agccacc |
| 157 | PL5 | UAS- | ggcctaactggccggtaccagcttgcatgcctgcaggtcggagtactgtcctccgag |
| | 51 | minB- | cggagtactgtcctccgagcggagtactgtcctccgagcggagtactgtcctccgag |
| | | FLUC_ | cggagtactgtcctccgagcggtgcgctcccgacatgccccgcggcgcgccattaac |
| | | no KPNI | cgccagatttgagtcgcgggacccgttggcagaggtgggctagcctcgaggatatca |
| | | | agatctggcctcggcggccaagcttggcaatccggtactgttggtaaagccacc |
| 158 | PL5 | TTF- | ggcctaactggccggtaccactagtggttttgtggggttttgtggggttttgtgggg |
| | 73 | 1_1_no | ttttgtggggttttgtggggttttgtggggttttgtggggttttgtggggttttgtg |
| | | space_ | gggttttgtggtgcgctcccgacatgccccgcggcgcgccattaaccgccagatttg |
| | | minBIRC5 | agtcgcgggacccgttggcagaggtgggctagcctcgaggatatcaagatctggcct |
| | | | cggcggccaagcttggcaatccggtactgttggtaaagccacc |
| 159 | PL5 | TTF- | ggcctaactggccggtaccactagtagccacttgaaattagccacttgaaattagcc |
| | 74 | 1_2_no | acttgaaattagccacttgaaattagccacttgaaattagccacttgaaattagcca |
| | | space_ | cttgaaatttgcgctcccgacatgccccgcggcgcgccattaaccgccagatttgag |
| | | minBIRC5 | tcgcgggacccgttggcagaggtgggctagcctcgaggatatcaagatctggcctcg |
| | | | gcggccaagcttggcaatccggtactgttggtaaagccacc |
| 160 | PL5 | TTF- | ggcctaactggccggtaccactagtctgggaacaagtgctgggaacaagtgctggga |
| | 75 | 1_3_no | acaagtgctgggaacaagtgctgggaacaagtgctgggaacaagtgctgggaacaag |
| | | space_ | tgctgggaacaagtgtgcgctcccgacatgccccgcggcgcgccattaaccgccaga |
| | | minBIRC5 | tttgagtcgcgggacccgttggcagaggtgggctagcctcgaggatatcaagatctg |
| | | | gcctcggcggccaagcttggcaatccggtactgttggtaaagccacc |
| 161 | PL5 | TTF- | ggcctaactggccggtaccactagtgactcctcaaggggactcctcaaggggactcc |
| | 76 | 1_4_no | tcaaggggactcctcaaggggactcctcaaggggactcctcaaggggactcctcaag |
| | | space_ | gggactcctcaagggtgcgctcccgacatgccccgcggcgcgccattaaccgccaga |
| | | minBIRC5 | tttgagtcgcgggacccgttggcagaggtgggctagcctcgaggatatcaagatctg |
| | | | gcctcggcggccaagcttggcaatccggtactgttggtaaagccacc |
| 162 | PL5 | TCF7_no | ggcctaactggccggtaccactagtcgggctttgatctttcgggctttgatctttcg |
| | 77 | space_ | ggctttgatctttcgggctttgatctttcgggctttgatctttcgggctttgatctt |
| | | minBIRC5 | tcgggctttgatcttttgcgctcccgacatgccccgcggcgcgccattaaccgccag |
| | | | atttgagtcgcgggacccgttggcagaggtgggctagcctcgaggatatcaagatct |
| | | | ggcctcggcggccaagcttggcaatccggtactgttggtaaagccacc |
| 163 | PL5 | TCF7:L2_ | ggcctaactggccggtaccactagtgcgctttgatgtgcggggcggccctttgaagt |
| | 78 | no | tggcgctttgatgtgcggggcggccctttgaagttggcgctttgatgtgcggggcgg |
| | | space_ | ccctttgaagttgtgcgctcccgacatgccccgcggcgcgccattaaccgccagatt |
| | | minBIRC5 | tgagtcgcgggacccgttggcagaggtgggctagcctcgaggatatcaagatctggc |
| | | | ctcggcggccaagcttggcaatccggtactgttggtaaagccacc |
| 164 | PL5 | MSC_no | ggcctaactggccggtaccactagtaacagctgttaacagctgttaacagctgttaa |
| | 79 | space_ | cagctgttaacagctgttaacagctgttaacagctgttaacagctgttaacagctgt |
| | | minBIRC5 | ttgcgctcccgacatgccccgcggcgcgccattaaccgccagatttgagtcgcggga |
| | | | cccgttggcagaggtgggctagcctcgaggatatcaagatctggcctcggcggccaa |
| | | | gcttggcaatccggtactgttggtaaagccacc |
| 165 | PL5 | ZEB1_no | ggcctaactggccggtaccactagtcacctgcacctgcacctgcacctgcacctgca |
| | 80 | space_ | cctgcacctgcacctgcacctgcacctgcacctgcacctgtgcgctcccgacatgcc |
| | | minBIRC5 | ccgcggcgcgccattaaccgccagatttgagtcgcgggacccgttggcagaggtggg |
| | | | ctagcctcgaggatatcaagatctggcctcggcggccaagcttggcaatccggtact |
| | | | gttggtaaagccacc |
| 166 | PL5 | MAX_MYC_ | ggcctaactggccggtaccactagtagttcaacacgtggtctgggagttcaacacgt |
| | 81 | no | ggtctgggagttcaacacgtggtctgggagttcaacacgtggtctgggagttcaaca |
| | | space_ | cgtggtctgggtgcgctcccgacatgccccgcggcgcgccattaaccgccagatttg |
| | | minBIRC5 | agtcgcgggacccgttggcagaggtgggctagcctcgaggatatcaagatctggcct |
| | | | cggcggccaagcttggcaatccggtactgttggtaaagccacc |
| 167 | PL5 | GATA6_ | ggcctaactggccggtaccactagtgacagataagaaagacagataagaaagacaga |
| | 82 | no | taagaaagacagataagaaagacagataagaaagacagataagaaagacagataaga |
| | | space_ | aagacagataagaaatgcgctcccgacatgccccgcggcgcgccattaaccgccaga |
| | | minBIRC5 | tttgagtcgcgggacccgttggcagaggtgggctagcctcgaggatatcaagatctg |
| | | | gcctcggcggccaagcttggcaatccggtactgttggtaaagccacc |
| 168 | PL5 | GATA1- | ggcctaactggccggtaccactagtttctaatctatttctaatctatttctaatcta |
| | 83 | BIRC5core | tttctaatctatttctaatctatttctaatctatttctaatctatttctaatctatt |
| | | | tctaatctattgcgctcccgacatgccccgcggcgcgccattaaccgccagatttga |
| | | | gtcgcgggacccgttggcagaggtgggctagcctcgaggatatcaagatctggcctc |
| | | | ggcggccaagcttggcaatccggtactgttggtaaagccacc |
| 169 | PL5 | FOSL1_ | ggcctaactggccggtaccactagtggtgactcatgggtgactcatgggtgactcat |
| | 84 | no | gggtgactcatgggtgactcatgggtgactcatgggtgactcatgggtgactcatgg |
| | | space_ | gtgactcatgtgcgctcccgacatgccccgcggcgcgccattaaccgccagatttga |
| | | minBIRC5 | gtcgcgggacccgttggcagaggtgggctagcctcgaggatatcaagatctggcctc |
| | | | ggcggccaagcttggcaatccggtactgttggtaaagccacc |
| 170 | PL5 | STAT3_ | ggcctaactggccggtaccactagtcttctgggaaacttctgggaaacttctgggaa |
| | 85 | no | acttctgggaaacttctgggaaacttctgggaaacttctgggaaacttctgggaaac |
| | | space_ | ttctgggaaatgcgctcccgacatgccccgcggcgcgccattaaccgccagatttga |
| | | minBIRC5 | gtcgcgggacccgttggcagaggtgggctagcctcgaggatatcaagatctggcctc |
| | | | ggcggccaagcttggcaatccggtactgttggtaaagccacc |
| 171 | PL5 | STAT: | ggcctaactggccggtaccactagtaattcttagaaataaattcttagaaataaatt |
| | 86 | STAT_no | cttagaaataaattcttagaaataaattcttagaaataaattcttagaaataaattc |
| | | space_ | ttagaaatatgcgctcccgacatgccccgcggcgcgccattaaccgccagatttgag |
| | | minBIRC5 | tcgcgggacccgttggcagaggtgggctagcctcgaggatatcaagatctggcctcg |
| | | | gcggccaagcttggcaatccggtactgttggtaaagccacc |
| 172 | PL5 | SOX9_no | ggcctaactggccggtaccactagtaaaacaaaggatcctttgttttaaaacaaagg |
| | 87 | space_ | atcctttgttttaaaacaaaggatcctttgttttaaaacaaaggatcctttgtttta |
| | | minBIRC5 | aaacaaaggatcctttgttttctgcgctcccgacatgccccgcggcgcgccattaac |
| | | | cgccagatttgagtcgcgggacccgttggcagaggtgggctagcctcgaggatatca |
| | | | agatctggcctcggcggccaagcttggcaatccggtactgttggtaaagccacc |
| 173 | PL5 | HNF4_no | ggcctaactggccggtaccactagtaaagtccaagtccaaaagtccaagtccaaaag |
| | 88 | space_ | tccaagtccaaaagtccaagtccaaaagtccaagtccaaaagtccaagtccaaaagt |
| | | minBIRC5 | ccaagtccatgcgctcccgacatgccccgcggcgcgccattaaccgccagatttgag |
| | | | tcgcgggacccgttggcagaggtgggctagcctcgaggatatcaagatctggcctcg |
| | | | gcggccaagcttggcaatccggtactgttggtaaagccacc |
| 174 | PL5 | TTF- | ggcctaactggccggtaccactagtggttttgtggagaggttttgtggtcgggtttt |
| | 89 | 1_1_3 bp | gtgggacggttttgtggctaggttttgtggactggttttgtggtgcggttttgtggg |
| | | space_ | taggttttgtggtgcgctcccgacatgccccgcggcgcgccattaaccgccagattt |
| | | minBIRC5 | gagtcgcgggacccgttggcagaggtgggctagcctcgaggatatcaagatctggcc |
| | | | tcggcggccaagcttggcaatccggtactgttggtaaagccacc |
| 175 | PL5 | TTF- | ggcctaactggccggtaccactagtagccacttgaaattagaagccacttgaaattt |
| | 90 | 1_2_3 bp | cgagccacttgaaattgacagccacttgaaattctaagccacttgaaattactagcc |
| | | space_ | acttgaaatttgcgctcccgacatgccccgcggcgcgccattaaccgccagatttga |
| | | nmiBIRC5 | gtcgcgggacccgttggcagaggtgggctagcctcgaggatatcaagatctggcctc |
| | | | ggcggccaagcttggcaatccggtactgttggtaaagccacc |
| 176 | PL5 | TTF- | ggcctaactggccggtaccactagtctgggaacaagtgagactgggaacaagtgtcg |
| | 91 | 1_3_3 bp | ctgggaacaagtggacctgggaacaagtgctactgggaacaagtgactctgggaaca |
| | | space_ | agtgtgcctgggaacaagtgtgcgctcccgacatgccccgcggcgcgccattaaccg |
| | | minBIRC5 | ccagatttgagtcgcgggacccgttggcagaggtgggctagcctcgaggatatcaag |
| | | | atctggcctcggcggccaagcttggcaatccggtactgttggtaaagccacc |
| 177 | PL5 | TTF- | ggcctaactggccggtaccactagtgactcctcaagggagagactcctcaagggtcg |
| | 92 | 1_4_3 bp | gactcctcaaggggacgactcctcaagggctagactcctcaagggactgactcctca |
| | | space_ | agggtgcgactcctcaagggtgcgctcccgacatgccccgcggcgcgccattaaccg |
| | | minBIRC5 | ccagatttgagtcgcgggacccgttggcagaggtgggctagcctcgaggatatcaag |
| | | | atctggcctcggcggccaagcttggcaatccggtactgttggtaaagccacc |
| 178 | PL5 | TCF7_3 bp | ggcctaactggccggtaccactagtccggctttgatctttagacgggctttgatctt |
| | 93 | space_ | ttcgcgggctttgatctttgaccgggctttgatctttctacgggctttgatctttac |
| | | minBIRC5 | tcgggctttgatcttttgcgctcccgacatgccccgcggcgcgccattaaccgccag |
| | | | atttgagtcgcgggacccgttggcagaggtgggctagcctcgaggatatcaagatct |
| | | | ggcctcggcggccaagcttggcaatccggtactgttggtaaagccacc |
| 179 | PL5 | TCF7:L2_ | ggcctaactggccggtaccactagtgcgctttgatgtgcggggcggccctttgaagt |
| | 94 | 3 bp | tgagagcgctttgatgtgcggggcggccctttgaagttgtcggcgctttgatgtgcg |
| | | space_ | gggcggccctttgaagttgtgcgctcccgacatgccccgcggcgcgccattaaccgc |
| | | minBIRC5 | cagatttgagtcgcgggacccgttggcagaggtgggctagcctcgaggatatcaaga |
| | | | tctggcctcggcggccaagcttggcaatccggtactgttggtaaagccacc |
| 180 | PL5 | MSC_3 bp | ggcctaactggccggtaccactagtaacagctgttagaaacagctgtttcgaacagc |
| | 95 | space_ | tgttgacaacagctgttctaaacagctgttactaacagctgtttgcaacagctgttg |
| | | minBIRC5 | taaacagctgtttgcgctcccgacatgccccgcggcgcgccattaaccgccagattt |
| | | | gagtcgcgggacccgttggcagaggtgggctagcctcgaggatatcaagatctggcc |
| | | | tcggcggccaagcttggcaatccggtactgttggtaaagccacc |
| 181 | PL5 | ZEB1 3 bp | ggcctaactggccggtaccactagtcacctgagacacctgtcgcacctggaccacct |
| | 96 | space_ | gctacacctgactcacctgtgccacctgagacacctgtcgcacctggaccacctgtg |
| | | minBIRC5 | cgctcccgacatgccccgcggcgcgccattaaccgccagatttgagtcgcgggaccc |
| | | | gttggcagaggtgggctagcctcgaggatatcaagatctggcctcggcggccaagct |
| | | | tggcaatccggtactgttggtaaagccacc |
| 182 | PL5 | MAX_MYC_ | ggcctaactggccggtaccactagtagttcaacacgtggtctgggagaagttcaaca |
| | 97 | 3 bp | cgtggtctgggtcgagttcaacacgtggtctggggacagttcaacacgtggtctggg |
| | | space_ | ctaagttcaacacgtggtctgggtgcgctcccgacatgccccgcggcgcgccattaa |
| | | minBIRC5 | ccgccagatttgagtcgcgggacccgttggcagaggtgggctagcctcgaggatatc |
| | | | aagatctggcctcggcggccaagcttggcaatccggtactgttggtaaagccacc |
| 183 | PL5 | GATA6_ | ggcctaactggccggtaccactagtgacagataagaaaagagacagataagaaatcg |
| | 98 | 3 bp | gacagataagaaagacgacagataagaaactagacagataagaaaactgacagataa |
| | | space_ | gaaatgcgacagataagaaatgcgctcccgacatgccccgcggcgcgccattaaccg |
| | | minBIRC5 | ccagatttgagtcgcgggacccgttggcagaggtgggctagcctcgaggatatcaag |
| | | | atctggcctcggcggccaagcttggcaatccggtactgttggtaaagccacc |
| 184 | PL5 | GATA1_ | ggcctaactggccggtaccactagtttctaatctatagattctaatctattcgttct |
| | 99 | 3 bp | aatctatgacttctaatctatctattctaatctatactttctaatctattgcttcta |
| | | space_ | atctattgcgctcccgacatgccccgcggcgcgccattaaccgccagatttgagtcg |
| | | minBIRC5 | cgggacccgttggcagaggtgggctagcctcgaggatatcaagatctggcctcggcg |
| | | | gccaagcttggcaatccggtactgttggtaaagccacc |
| 185 | PL6 | FOSL1_ | ggcctaactggccggtaccactagtggtgactcatgagaggtgactcatgtcgggtg |
| | 00 | 3 bp | actcatggacggtgactcatgctaggtgactcatgactggtgactcatgtgcggtga |
| | | space_ | ctcatgctgcgctcccgacatgccccgcggcgcgccattaaccgccagatttgagtc |
| | | nmiBIRC5 | gcgggacccgttggcagaggtgggctagcctcgaggatatcaagatctggcctcggc |
| | | | ggccaagcttggcaatccggtactgttggtaaagccacc |
| 186 | PL6 | STAT3_ | ggcctaactggccggtaccactagtcttctgggaaaagacttctgggaaatcgcttc |
| | 01 | 3 bp | tgggaaagaccttctgggaaactacttctgggaaaactcttctgggaaatgccttct |
| | | space_ | gggaaatgcgctcccgacatgccccgcggcgcgccattaaccgccagatttgagtcg |
| | | minBIRC5 | cgggacccgttggcagaggtgggctagcctcgaggatatcaagatctggcctcggcg |
| | | | gccaagcttggcaatccggtactgttggtaaagccacc |
| 187 | PL6 | STAT: | ggcctaactggccggtaccactagtaattcttagaaataagaaattcttagaaatat |
| | 02 | STAT_3 bp | cgaattcttagaaatagacaattcttagaaatactaaattcttagaaataactaatt |
| | | space_ | cttagaaatatgcgctcccgacatgccccgcggcgcgccattaaccgccagatttga |
| | | minBIRC5 | gtcgcgggacccgttggcagaggtgggctagcctcgaggatatcaagatctggcctc |
| | | | ggcggccaagcttggcaatccggtactgttggtaaagccacc |
| 188 | PL6 | SOX9_ | ggcctaactggccggtaccactagtaaaacaaaggatcctttgttttagaaaaacaa |
| | 03 | 3 bp | aggatcctttgtttttcgaaaacaaaggatcctttgttttgacaaaacaaaggatcc |
| | | space_ | tttgtttttgcgctcccgacatgccccgcggcgcgccattaaccgccagatttgagt |
| | | nBIRC5 | cgcgggacccgttggcagaggtgggctagcctcgaggatatcaagatctggcctcgg |
| | | mi | cggccaagcttggcaatccggtactgttggtaaagccacc |
| 189 | PL6 | HNF4_ | ggcctaactggccggtaccactagtaaagtccaagtccaagaaaagtccaagtccat |
| | 04 | 3 bp | cgaaagtccaagtccagacaaagtccaagtccactaaaagtccaagtccaactaaag |
| | | space_ | tccaagtccatgcgctcccgacatgccccgcggcgcgccattaaccgccagatttga |
| | | minBIRC5 | gtcgcgggacccgttggcagaggtgggctagcctcgaggatatcaagatctggcctc |
| | | | ggcggccaagcttggcaatccggtactgttggtaaagccacc |
| 190 | PL6 | STAT: | ggcctaactggccggtaccactagtaattcttagaaataaattcttagaaataaatt |
| | 05 | STAT_no | cttagaaataaattcttagaaataaattcttagaaataaattcttagaaataaattc |
| | | space_ | ttagaaatatgcgctcccgacatgtcccgcggcgcgccattaaccgccagatttgag |
| | | minBIRC52 | tcgcgggacccgttggcagaggtgggctagcctcgaggatatcaagatctggcctcg |
| | | w extra | gcggccaagcttggcaatccggtactgttggtaaagccaccatcctcgaggatatca |
| | | insert | agatctggcctcggcggccaagcttggcaatccggtactgttggtaaagccacc |
| 191 | PL6 | HOXA13_ | ggcctaactggccggtaccactagtccaataaaaaccaataaaaaccaataaaaacc |
| | 16 | no | aataaaaaccaataaaaaccaataaaaaccaataaaaaccaataaaaaccaataaaa |
| | | space_minB | atgcgctcccgacatgccccgcggcgcgccattaaccgccagatttgagtcgcggga |
| | | | cccgttggcagaggtgggctagcctcgaggatatcaagatctggcctcggcggccaa |
| | | | gcttggcaatccggtactgttggtaaagccacc |
| 193 | PL6 | FOXM1_ | ggcctaactggccggtaccactagttgtttacttatgtttacttatgtttacttatg |
| | 35 | no | tttacttatgtttacttatgtttacttatgtttacttatgtttacttatgtttactt |
| | | space_ | atgcgctcccgacatgccccgcggcgcgccattaaccgccagatttgagtcgcggga |
| | | coreBIRC5 | cccgttggcagaggtgggctagcctcgaggatatcaagatctggcctcggcggccaa |
| | | | gcttggcaatccggtactgttggtaaagccacc |
| 194 | PL6 | E2F2_no | ggcctaactggccggtaccactagtaaaatggcgccattttaaaatggcgccatttt |
| | 36 | space_ | aaaatggcgccattttaaaatggcgccattttaaaatggcgccattttaaaatggcg |
| | | coreBIRC5 | ccatttttgcgctcccgacatgccccgcggcgcgccattaaccgccagatttgagtc |
| | | | gcgggacccgttggcagaggtgggctagcctcgaggatatcaagatctggcctcggc |
| | | | ggccaagcttggcaatccggtactgttggtaaagccacc |
| 195 | PL6 | RUNX1_ | ggcctaactggccggtaccactagttattgtggttatattgtggttatattgtggtt |
| | 37 | no | atattgtggttatattgtggttatattgtggttatattgtggttatattgtggttat |
| | | space_ | gcgctcccgacatgccccgcggcgcgccattaaccgccagatttgagtcgcgggacc |
| | | rcoeBIRC5 | cgttggcagaggtgggctagcctcgaggatatcaagatctggcctcggcggccaagc |
| | | | ttggcaatccggtactgttggtaaagccacc |
| 196 | PL6 | SOX4_no | ggcctaactggccggtaccactagtgaacaattgcagtgttgaacaattgcagtgtt |
| | 38 | space_ | gaacaattgcagtgttgaacaattgcagtgttgaacaattgcagtgttgaacaattg |
| | | coreBIRC5 | cagtgttgaacaattgcagtgtttgcgctcccgacatgccccgcggcgcgccattaa |
| | | | ccgccagatttgagtcgcgggacccgttggcagaggtgggctagcctcgaggatatc |
| | | | aagatctggcctcggcggccaagcttggcaatccggtactgttggtaaagccacc |
| 197 | PL6 | RREB1_ | ggcctaactggccggtaccactagtccccaaaccaccccccccccccccaaaccacc |
| | 39 | no | ccccccccccccaaaccaccccccccccccccaaaccaccccccccccccccaaacc |
| | | space_ | acccccccccctgcgctcccgacatgccccgcggcgcgccattaaccgccagatttg |
| | | coreBIRC5 | agtcgcgggacccgttggcagaggtgggctagcctcgaggatatcaagatctggcct |
| | | | cggcggccaagcttggcaatccggtactgttggtaaagccacc |
| 198 | PL6 | ETV4_no | CACTAGTACCGGAAGTAACCGGAAGTAACCGGAAGTAACCGGAAGTAACCGGAAGTA |
| | 40 | space_ | ACCGGAAGTAACCGGAAGTAACCGGAAGTAACCGGAAGTAtgcgctcccgacatgcc |
| | | coreBIRC5 | ccgcggcgcgccattaaccgccagatttgagtcgcgggacccgttggcagaggtggg |
| | | | ctagcctcgaggatatcaagatctggcctcggcggccaagcttggcaatccggtact |
| | | | gttggtaaagccacc |
| 199 | PL6 | HES6_no | ggcctaactggccggtaccactagtggcacgtgttggcacgtgttggcacgtgttgg |
| | 41 | space_ | cacgtgttggcacgtgttggcacgtgttggcacgtgttggcacgtgttggcacgtgt |
| | | coreBIRC5 | ttgcgctcccgacatgccccgcggcgcgccattaaccgccagatttgagtcgcggga |
| | | | cccgttggcagaggtgggctagcctcgaggatatcaagatctggcctcggcggccaa |
| | | | gcttggcaatccggtactgttggtaaagccacc |
| 200 | PL6 | ASCL1_ | ggcctaactggccggtaccactagtcgagcagctggtgcgagcagctggtgcgagca |
| | 42 | no | gctggtgcgagcagctggtgcgagcagctggtgcgagcagctggtgcgagcagctgg |
| | | space_ | tgtgcgctcccgacatgccccgcggcgcgccattaaccgccagatttgagtcgcggg |
| | | coreBIRC5 | acccgttggcagaggtgggctagcctcgaggatatcaagatctggcctcggcggcca |
| | | | agcttggcaatccggtactgttggtaaagccacc |
| 201 | PL6 | TWIST1_ | ggcctaactggccggtaccactagttccagatgtttccagatgtttccagatgtttc |
| | 43 | no | cagatgtttccagatgtttccagatgtttccagatgtttccagatgtttgcgctccc |
| | | space_ | gacatgccccgcggcgcgccattaaccgccagatttgagtcgcgggacccgttggca |
| | | coreBIRC5 | gaggtgggctagcctcgaggatatcaagatctggcctcggcggccaagcttggcaat |
| | | | ccggtactgttggtaaagccacc |
| 202 | PL6 | FOXA3_ | ggcctaactggccggtaccactagtatagtaaacaatagtaaacaatagtaaacaat |
| | 44 | no | agtaaacaatagtaaacaatagtaaacaatagtaaacaatagtaaacatgcgctccc |
| | | space_ | gacatgccccgcggcgcgccattaaccgccagatttgagtcgcgggacccgttggca |
| | | coreBIRC5 | gaggtgggctagcctcgaggatatcaagatctggcctcggcggccaagcttggcaat |
| | | | ccggtactgttggtaaagccacc |
| 203 | PL6 | PITX2_no | ggcctaactggccggtaccactagttaatccctaatccctaatccctaatccctaat |
| | 45 | space_ | ccctaatccctaatccctaatccctaatccctaatccctaatccctgcgctcccgac |
| | | coreBIRC5 | atgccccgcggcgcgccattaaccgccagatttgagtcgcgggacccgttggcagag |
| | | | gtgggctagcctcgaggatatcaagatctggcctcggcggccaagcttggcaatccg |
| | | | gtactgttggtaaagccacc |
| 204 | PL6 | HOXB2_ | ggcctaactggccggtaccactagtctaattaactaattaactaattaactaattaa |
| | 46 | no | ctaattaactaattaactaattaactaattaactaattaactaattaatgcgctccc |
| | | space_ | gacatgccccgcggcgcgccattaaccgccagatttgagtcgcgggacccgttggca |
| | | coreBIRC5 | gaggtgggctagcctcgaggatatcaagatctggcctcggcggccaagcttggcaat |
| | | | ccggtactgttggtaaagccacc |
| 205 | PL6 | EN2_no | ggcctaactggccggtaccactagtcccaattagccccaattagccccaattagccc |
| | 47 | space_ | caattagccccaattagccccaattagccccaattagccccaattagctgcgctccc |
| | | coreBIRC5 | gacatgccccgcggcgcgccattaaccgccagatttgagtcgcgggacccgttggca |
| | | | gaggtgggctagcctcgaggatatcaagatctggcctcggcggccaagcttggcaat |
| | | | ccggtactgttggtaaagccacc |
| 206 | PL6 | DLX4_no | ggcctaactggccggtaccactagtcaattacaattacaattacaattacaattaca |
| | 48 | space_ | attacaattacaattacaattacaattacaattacaattatgcgctcccgacatgcc |
| | | rcoeBIRC5 | ccgcggcgcgccattaaccgccagatttgagtcgcgggacccgttggcagaggtggg |
| | | | ctagcctcgaggatatcaagatctggcctcggcggccaagcttggcaatccggtact |
| | | | gttggtaaagccacc |
| 207 | PL6 | GRHL1_ | ggcctaactggccggtaccactagtaaaaccggttttaaaaccggttttaaaaccgg |
| | 49 | no | ttttaaaaccggttttaaaaccggttttaaaaccggttttaaaaccggttttaaaac |
| | | space_ | cggtttttgcgctcccgacatgccccgcggcgcgccattaaccgccagatttgagtc |
| | | coreBIRC5 | gcgggacccgttggcagaggtgggctagcctcgaggatatcaagatctggcctcggc |
| | | | ggccaagcttggcaatccggtactgttggtaaagccacc |
| 208 | PL6 | FOXM1_ | ggcctaactggccggtaccactagttgtttacttaagatgtttacttatcgtgttta |
| | 50 | 3 bp | cttagactgtttacttactatgtttacttaacttgtttacttatgctgtttacttat |
| | | space_ | gcgctcccgacatgccccgcggcgcgccattaaccgccagatttgagtcgcgggacc |
| | | coreBIRC5 | cgttggcagaggtgggctagcctcgaggatatcaagatctggcctcggcggccaagc |
| | | | ttggcaatccggtactgttggtaaagccacc |
| 209 | PL6 | E2F2_3 bp | ggcctaactggccggtaccactagtaaaatggcgccatttttcgaaaatggcgccat |
| | 51 | space_ | tttgacaaaatggcgccattttctaaaaatggcgccattttactaaaatggcgccat |
| | | coreBIRC5 | ttttgcaaaatggcgccatttttgcgctcccgacatgccccgcggcgcgccattaac |
| | | | cgccagatttgagtcgcgggacccgttggcagaggtgggctagcctcgaggatatca |
| | | | agatctggcctcggcggccaagcttggcaatccggtactgttggtaaagccacc |
| 210 | PL6 | RUNX1_ | ggcctaactggccggtaccactagttattgtggttatcgtattgtggttagactatt |
| | 52 | 3 bp | gtggttactatattgtggttaacttattgtggttatgctattgtggttatgcgctcc |
| | | space_ | cgacatgccccgcggcgcgccattaaccgccagatttgagtcgcgggacccgttggc |
| | | coreBIRC5 | agaggtgggctagcctcgaggatatcaagatctggcctcggcggccaagcttggcaa |
| | | | tccggtactgttggtaaagccacc |
| 211 | PL6 | SOX4_3 bp | ggcctaactggccggtaccactagtgaacaattgcagtgttgacgaacaattgcagt |
| | 53 | space_ | gttctagaacaattgcagtgttactgaacaattgcagtgtttgcgaacaattgcagt |
| | | coreBIRC5 | gtttgcgctcccgacatgccccgcggcgcgccattaaccgccagatttgagtcgcgg |
| | | | gacccgttggcagaggtgggctagcctcgaggatatcaagatctggcctcggcggcc |
| | | | aagcttggcaatccggtactgttggtaaagccacc |
| 212 | PL6 | RREB1_ | ggcctaactggccggtaccactagtccccaaaccaccccccccccgacccccaaacc |
| | 54 | 3 bp | accccccccccctaccccaaaccaccccccccccactccccaaaccacccccccccc |
| | | space_ | tgcgctcccgacatgccccgcggcgcgccattaaccgccagatttgagtcgcgggac |
| | | coreBIRC5 | ccgttggcagaggtgggctagcctcgaggatatcaagatctggcctcggcggccaag |
| | | | cttggcaatccggtactgttggtaaagccacc |
| 213 | PL6 | ETV4_3 bp | ggcctaactggccggtaccactagtaccggaagtaagaaccggaagtatcgaccgga |
| | 55 | space_ | agtagacaccggaagtactaaccggaagtaactaccggaagtatgcaccggaagtat |
| | | coreBIRC5 | gcgctcccgacatgccccgcggcgcgccattaaccgccagatttgagtcgcgggacc |
| | | | cgttggcagaggtgggctagcctcgaggatatcaagatctggcctcggcggccaagc |
| | | | ttggcaatccggtactgttggtaaagccacc |
| 214 | PL6 | HES6_3 bp | ggcctaactggccggtaccactagtggcacgtgttagaggcacgtgtttcgggcacg |
| | 56 | space_ | tgttgacggcacgtgttctaggcacgtgttactggcacgtgtttgcggcacgtgttt |
| | | coreBIRC5 | gcgctcccgacatgccccgcggcgcgccattaaccgccagatttgagtcgcgggacc |
| | | | cgttggcagaggtgggctagcctcgaggatatcaagatctggcctcggcggccaagc |
| | | | ttggcaatccggtactgttggtaaagccacc |
| 215 | PL6 | ASCL1_ | ggcctaactggccggtaccactagtcgagcagctggtgagacgagcagctggtgtcg |
| | 57 | 3 bp | cgagcagctggtggaccgagcagctggtgctacgagcagctggtgactcgagcagct |
| | | space_ | ggtgtgcgctcccgacatgccccgcggcgcgccattaaccgccagatttgagtcgcg |
| | | coreBIRC5 | ggacccgttggcagaggtgggctagcctcgaggatatcaagatctggcctcggcggc |
| | | | caagcttggcaatccggtactgttggtaaagccacc |
| 216 | PL6 | TWIST1_ | ggcctaactggccggtaccactagttccagatgttagatccagatgtttcgtccaga |
| | 58 | 3 bp | tgttgactccagatgttctatccagatgttacttccagatgtttgctccagatgttt |
| | | space_ | gcgctcccgacatgccccgcggcgcgccattaaccgccagatttgagtcgcgggacc |
| | | coreBIRC5 | cgttggcagaggtgggctagcctcgaggatatcaagatctggcctcggcggccaagc |
| | | | ttggcaatccggtactgttggtaaagccacc |
| 217 | PL6 | FOXA3_ | ggcctaactggccggtaccactagtatagtaaacaagaatagtaaacatcgatagta |
| | 59 | 3 bp | aacagacatagtaaacactaatagtaaacaactatagtaaacatgcatagtaaacat |
| | | space_ | gcgctcccgacatgccccgcggcgcgccattaaccgccagatttgagtcgcgggacc |
| | | coreBIRC5 | cgttggcagaggtgggctagcctcgaggatatcaagatctggcctcggcggccaagc |
| | | | ttggcaatccggtactgttggtaaagccacc |
| 218 | PL6 | PITX2_3 bp | ggcctaactggccggtaccactagttaatcccagataatccctcgtaatcccgacta |
| | 60 | space_ | atcccctataatcccacttaatccctgctaatcccacttaatccctgctaatccctg |
| | | coreBIRC5 | cgctcccgacatgccccgcggcgcgtcattaaccgccagatttgagtcgcgggaccc |
| | | | gttggcagaggtgggctagcctcgaggatatcaagatctggcctcggcggccaagct |
| | | | tggcaatccggtactgttggtaaagccacc |
| 219 | PL6 | HOXB2_ | ggcctaactggccggtaccactagtctaattaaagactaattaatcgctaattaaga |
| | 61 | 3 bp | cctaattaactactaattaaactctaattaatgcctaattaaactctaattaatgcg |
| | | space_ | ctcccgacatgccccgcggcgcgccattaaccgccagatttgagtcgcgggacccgt |
| | | coreBIRC5 | tggcagaggtgggctagcctcgaggatatcaagatctggcctcggcggccaagcttg |
| | | | gcaatccggtactgttggtaaagccacc |
| 220 | PL6 | EN2_3 bp | ggcctaactggccggtaccactagtcccaattagcagacccaattagctcgcccaat |
| | 62 | space_ | tagcgaccccaattagcctacccaattagcactcccaattagctgccccaattagct |
| | | coreBIRC5 | gcgctcccgacatgccctgcggcgcgccattaaccgccagatttgagtcgcgggacc |
| | | | cgttggcagaggtgggctagcctcgaggatatcaagatctggcctcggcggccaagc |
| | | | ttggcaatccggtactgttggtaaagccacc |
| 221 | PL6 | DLX4_3 bp | ggcctaactggccggtaccactagtcaattaagacaattatcgcaattagaccaatt |
| | 63 | space_ | actacaattaactcaattatgccaattaactcaattatgccaattaagacaattatg |
| | | coreBIRC5 | cgctcccgacatgccccgcggcgtgccattaaccgccagatttgagtcgcgggaccc |
| | | | gttggcagaggtgggctagcctcgaggatatcaagatctggcctcggcggccaagct |
| | | | tggcaatccggtactgttggtaaagccacc |
| 222 | PL6 | GRHL1_ | ggcctaactggccggtaccactagtaaaaccggttttagaaaaaccggtttttcgaa |
| | 64 | 3bp | aaccggttttgacaaaaccggttttctaaaaaccggttttactaaaaccggtttttg |
| | | space_ | caaaaccggtttttgcgctcccgacatgccccgcggcgcgccattaaccgccagatt |
| | | coreBIRC5 | tgagtcgcgggacccgttggcagaggtgggctagcctcgaggatatcaagatctggc |
| | | | ctcggcggccaagcttggcaatccggtactgttggtaaagccacc |
| 223 | PL6 | FOSL1- | ggcctaactggccggtaccactagtggtgactcatgggtgactcatgggtgactcat |
| | 69 | 5X_ | gggtgactcatgggtgactcatgtgcgctcccgacatgccccgcggcgcgccattaa |
| | | BIRC5core | ccgccagatttgagtcgcgggacccgttggcagaggtgggctagcctcgaggatatc |
| | | | aagatctggcctcggcggccaagcttggcaatccggtactgttggtaaagccacc |
| 224 | PL6 | FOSL1- | ggcctaactggccggtaccactagtggtgactcatgggtgactcatgggtgactcat |
| | 72 | 11X_ | gggtgactcatgggtgactcatgggtgactcatgggtgactcatgggtgactcatgg |
| | | BIRC5core | gtgactcatgggtgactcatgggtgactcatgtgcgctcccgacatgccccgcggcg |
| | | | cgccattaaccgccagatttgagtcgcgggacccgttggcagaggtgggctagcctc |
| | | | gaggatatcaagatctggcctcggcggccaagcttggcaatccggtactgttggtaa |
| | | | agccacc |
| 225 | PL6 | FOSL1- | ggcctaactggccggtaccactagtggtgactcatgggtgactcatgggtgactcat |
| | 73 | 7X_ | gggtgactcatgggtgactcatgggtgactcatgggtgactcatgtgcgctcccgac |
| | | BIRC5core | atgccccgcggcgcgccattaaccgccagatttgagtcgcgggacccgttggcagag |
| | | | gtgggctagcctcgaggatatcaagatctggcctcggcggccaagcttggcaatccg |
| | | | gtactgttggtaaagccacc |
| 226 | PL6 | FOSL1_ | ggcctaactggccggtaccactagtggtgactcatgggtgactcatgggtgactcat |
| | 74 | no | gggtgactcatgggtgactcatgggtgactcatgggtgactcatgggtgactcatgg |
| | | space_ | gtgactcatgcggcgcgccattaaccgccagatttgagtcgcgggacccgttggcag |
| | | nop53_ | aggtgggctagcctcgaggatatcaagatctggcctcggcggccaagcttggcaatc |
| | | BIRC5core | cggtactgttggtaaagccacc |
| 227 | PL6 | FOSL1_ | ggcctaactggccggtaccactagtggtgactcatgggtgactcatgggtgactcat |
| | 75 | TATATSS_ | gggtgactcatgggtgactcatgggtgactcatgggtgactcatgggtgactcatgg |
| | | 10 bp | gtgactcatgcggtgctagctataaaaggccagcagcagcctgaccacatctcatcc |
| | | spacing | tcctcgaggatatcaagatctggcctcggcggccaagcttggcaatccggtactgtt |
| | | | ggtaaagccacc |
| 228 | PL6 | FOSL1_ | ggcctaactggccggtaccactagtggtgactcatgggtgactcatgggtgactcat |
| | 76 | TATATSS_ | gggtgactcatgggtgactcatgggtgactcatgggtgactcatgggtgactcatgg |
| | | no | gtgactcatgtataaaaggccagcagcagcctgaccacatctcatcctcctcgagga |
| | | spacing | tatcaagatctggcctcggcggccaagcttggcaatccggtactgttggtaaagcca |
| | | | CC |
| 229 | PL6 | FOSL1_ | ggcctaactggccggtaccactagtggtgactcatgggtgactcatgggtgactcat |
| | 85 | TATATSS_ | gggtgactcatgggtgactcatgggtgactcatgggtgactcatgggtgactcatgg |
| | | 25 bp | gtgactcatgacatctttcagggaccggtgctagctataaaaggccagcagcagcct |
| | | spacing | gaccacatctcatcctcctcgaggatatcaagatctggcctcggcggccaagcttgg |
| | | | caatccggtactgttggtaaagccacc |
| 230 | PL6 | FOSL1_ | ggcctaactggccggtaccactagtggtgactcatgggtgactcatgggtgactcat |
| | 86 | TATATSS_ | gggtgactcatgggtgactcatgggtgactcatgggtgactcatgggtgactcatgg |
| | | 50 bp | gtgactcatgtggctattagcagtaccgcttagacacatctttcagggaccggtgct |
| | | spacing | agctataaaaggccagcagcagcctgaccacatctcatcctcctcgaggatatcaag |
| | | | atctggcctcggcggccaagcttggcaatccggtactgttggtaaagccacc |
| 231 | PL6 | Forkhead_ | ggcctaactggccggtaccactagtctgtttacctgtttacctgtttacctgtttac |
| | 89 | 7XFOSL1_ | ctgtttacggtgactcatgggtgactcatgggtgactcatgggtgactcatgggtga |
| | | BIRC5core | ctcatgggtgactcatgggtgactcatgggtgactcatgggtgactcatgtgcgctc |
| | | | ccgacatgccccgcggcgcgccattaaccgccagatttgagtcgcgggacccgttgg |
| | | | cagaggtgggctagcctcgaggatatcaagatctggcctcggcggccaagcttggca |
| | | | atccggtactgttggtaaagccacc |
| 232 | PL | Forkhead_ | ggcctaactggccggtaccactagtctgtttacagactgtttactcgctgtttacga |
| | 90 | 7XFOSL1_ | cctgtttacctactgtttacggtgactcatgggtgactcatgggtgactcatgggtg |
| | | BIRC5core_ | actcatgggtgactcatgggtgactcatgggtgactcatgggtgactcatgggtgac |
| | | 3 bp | tcatgtgcgctcccgacatgccccgcggcgcgccattaaccgccagatttgagtcgc |
| | | | gggacccgttggcagaggtgggctagcctcgaggatatcaagatctggcctcggcgg |
| | | | ccaagcttggcaatccggtactgttggtaaagccacc |
| 233 | PL8 | FOSL1_ | ggcctaactggccggtaccactagtggtgactcatgggtgactcatgggtgactcat |
| | 25 | 10 bp | gggtgactcatgggtgactcatgggtgactcatgggtgactcatgggtgactcatgg |
| | | spacer_ | gtgactcatgcataggcctctgaacaacgcgtcccgacatgccccgcggcgcgccat |
| | | coreBIRC5 | taaccgccagatttgagtcgcgggacccgttggcagaggtgggctagcctcgaggat |
| | | | atcaagatctggcctcggcggccaagcttggcaatccggtactgttggtaaagccac |
| | | | C |
| 234 | PL8 | FOSL1_ | ggcctaactggccggtaccactagtggtgactcatgggtgactcatgggtgactcat |
| | 26 | 30 bp | gggtgactcatgggtgactcatgggtgactcatgggtgactcatgggtgactcatgg |
| | | spacer_ | gtgactcatgcataggcctctgatagagctgcgatagaccaagacaacgcgtcccga |
| | | coreBIRC5 | catgccccgcggcgcgccattaaccgccagatttgagtcgcgggacccgttggcaga |
| | | | ggtgggctagcctcgaggatatcaagatctggcctcggcggccaagcttggcaatcc |
| | | | ggtactgttggtaaagccacc |
| 235 | PL8 | FOSL1_ | ggcctaactggccggtaccactagtggtgactcatgggtgactcatgggtgactcat |
| | 27 | 88 bp | gggtgactcatgggtgactcatgggtgactcatgggtgactcatgggtgactcatgg |
| | | spacer_ | gtgactcatgcatagaaacgacgcaatatctccatagggttaacggcggaacttgac |
| | | coreBIRC5 | ggcgtccattagccacttggtcatgggacagggggggaaaacggacaacgcgtcccg |
| | | | acatgccccgcggcgcgccattaaccgccagatttgagtcgcgggacccgttggcag |
| | | | aggtgggctagcctcgaggatatcaagatctggcctcggcggccaagcttggcaatc |
| | | | cggtactgttggtaaagccacc |
| 236 | PL8 | FOSL1_ | ggcctaactggccggtaccactagtggtgactcatgggtgactcatgggtgactcat |
| | 28 | Low_ | gggtgactcatgggtgactcatgggtgactcatgggtgactcatgggtgactcatgg |
| | | coreBIRC5 | gtgactcatgcataccggaagtacttgcgcaatgaccggaagtacaacgcgtcccga |
| | | | catgccccgcggcgcgccattaaccgccagatttgagtcgcgggacccgttggcaga |
| | | | ggtgggctagcctcgaggatatcaagatctggcctcggcggccaagcttggcaatcc |
| | | | ggtactgttggtaaagccacc |
| 237 | PL8 | FOSL1_ | ggcctaactggccggtaccactagtggtgactcatgggtgactcatgggtgactcat |
| | 29 | Medium_ | gggtgactcatgggtgactcatgggtgactcatgggtgactcatgggtgactcatgg |
| | | coreBIRC5 | gtgactcatgcatttgcgcaacaggggcggggtgatgacacagcaattcgcttgcgt |
| | | | gagaagagaccggaagtgagggactttccacatgacacagcaatacaacgcgtcccg |
| | | | acatgccccgcggcgcgccattaaccgccagatttgagtcgcgggacccgttggcag |
| | | | aggtgggctagcctcgaggatatcaagatctggcctcggcggccaagcttggcaatc |
| | | | cggtactgttggtaaagccacc |
| 238 | PL8 | FOSL1_ | ggcctaactggccggtaccactagtggtgactcatgggtgactcatgggtgactcat |
| | 30 | High_ | gggtgactcatgggtgactcatgggtgactcatgggtgactcatgggtgactcatgg |
| | | coreBIRC5 | gtgactcatgcatggggcggggtgatgacacagcaattcgggactttccacgcttgc |
| | | | gtgagaagagaccggaagtgaatgacacagcaattcgcttgcgtgagaagctgggac |
| | | | tttcctaggggcggggttgggactttccacatgacacagcaatacaacgcgtcccga |
| | | | catgccccgcggcgcgccattaaccgccagatttgagtcgcgggacccgttggcaga |
| | | | ggtgggctagcctcgaggatatcaagatctggcctcggcggccaagcttggcaatcc |
| | | | ggtactgttggtaaagccacc |
| 239 | PL8 | Low_ | ggcctaactggccggtaccactagtaccggaagtacttgcgcaatgaccggaagtac |
| | 31 | coreBIRC5 | aacgcgtcccgacatgccccgcggcgcgccattaaccgccagatttgagtcgcggga |
| | | | cccgttggcagaggtgggctagcctcgaggatatcaagatctggcctcggcggccaa |
| | | | gcttggcaatccggtactgttggtaaagccacc |
| 240 | PL8 | Medium_ | ggcctaactggccggtaccactagtttgcgcaacaggggcggggtgatgacacagca |
| | 32 | coreBIRC5 | attcgcttgcgtgagaagagaccggaagtgagggactttccacatgacacagcaata |
| | | | caacgcgtcccgacatgccccgcggcgcgccattaaccgccagatttgagtcgcggg |
| | | | acccgttggcagaggtgggctagcctcgaggatatcaagatctggcctcggcggcca |
| | | | agcttggcaatccggtactgttggtaaagccacc |
| 241 | PL8 | High_ | ggcctaactggccggtaccactagtggggcggggtgatgacacagcaattcgggact |
| | 33 | coreBIRC5 | ttccacgcttgcgtgagaagagaccggaagtgaatgacacagcaattcgcttgcgtg |
| | | | agaagctgggactttcctaggggcggggttgggactttccacatgacacagcaatac |
| | | | aacgcgtcccgacatgccccgcggcgcgccattaaccgccagatttgagtcgcggga |
| | | | cccgttggcagaggtgggctagcctcgaggatatcaagatctggcctcggcggccaa |
| | | | gcttggcaatccggtactgttggtaaagccacc |
| 242 | PL8 | FOSL1_ | ggcctaactggccggtaccactagtggtgactcatgggtgactcatgggtgactcat |
| | 34 | Tetramer | gggtgactcatgggtgactcatgggtgactcatgggtgactcatgggtgactcatgg |
| | | p53_ | gtgactcatgcatacaacgcgtcccgacatgccccgacatgcccatcgacatgcccc |
| | | coreBIRC5 | gacatgcccgcggcgcgccattaaccgccagatttgagtcgcgggacccgttggcag |
| | | | aggtgggctagcctcgaggatatcaagatctggcctcggcggccaagcttggcaatc |
| | | | cggtactgttggtaaagccacc |
| 243 | PL8 | FOSL1_ | ggcctaactggccggtaccactagtggtgactcatgggtgactcatgggtgactcat |
| | 35 | p53RE_ | gggtgactcatgggtgactcatgggtgactcatgggtgactcatgggtgactcatgg |
| | | coreBIRC5 | gtgactcatgcatgaattcggacatgcccgggcatgtccccagggacatgcccgggc |
| | | | atgtccccagagacatgtccagacatgtccccaggaacatgtcccaacatgttgtcc |
| | | | aggagacatgtccagacatgtccccaggaacatgtcccaacatgttgtactagtaca |
| | | | acgcgtcccgacatgccccgcggcgcgccattaaccgccagatttgagtcgcgggac |
| | | | ccgttggcagaggtgggctagcctcgaggatatcaagatctggcctcggcggccaag |
| | | | cttggcaatccggtactgttggtaaagccacc |
| 244 | PL8 | EN7R_ | ggcctaactggccggtacctgccactcaaagtggcacactccctgctcaggaggccg |
| | 36 | FOSL1_ | ggagggaggacacagccctggcaactcctctgccccggggggtcaggaaggggtcac |
| | | coreBIRC5 | cccacactccagaaccctacagaatgtggccttggcttttcccatcaagagctgggg |
| | | | aaagccaggccccgacttcattaccccctgcccccgtcccatgctcagtgggcccca |
| | | | tcgtgggtccatgccacactcccaactgagcagccccgcagccccgcgtgtcacaga |
| | | | catggggcctcctaattgctgctgaggtcccaatccctggctggacgtgcctg |
| 245 | PL8 | FOSL | ggcctaactggccggtaccactagtggtgactcatgggtgactcatgggtgactcat |
| | 58 | CS6X- | gggtgactcatgggtgactcatgggtgactcatgggtgactcatgggtgactcatga |
| | | BIRC5core | ctagtgtccccacccacacattcctgtccccacccacacattcctgtccccacccac |
| | | | acattcctgtccccacccacacattcctgtccccacccacacattcctgtccccacc |
| | | | cacacattcctgtgcgctcccgacatgccccgcggcgcgccattaaccgccagattt |
| | | | gagtcgcgggacccgttggcagaggtgggctagcctcgaggatatcaagatctggcc |
| | | | tcggcggccaagcttggcaatccggtactgttggtaaagccacc |
| 246 | PL8 | pGL4.10- | ggcctaactggccggtaccaagacaggttgtcctcccaggggatgggggtccatcca |
| | 80 | coreCEACAM5_ | ccttgccgaaaagatttgtctgaggaactgaaaatagaagggaaaaaagaggaggga |
| | | 1 | caaaagaggcagaaatgagaggggaggggacagaggacacctgaataaagaccacac |
| | | | ccatgacccacgtgatgctgagaagtactcctgccctaggaagagactcagggcaga |
| | | | gggaggaaggacagcagaccagacagtcacagcagccttgacaaaacgttcctggaa |
| | | | ctaccggtgctagcctcgaggatatcaagatctggcctcggcggccaagcttggcaa |
| | | | tccggtactgttggtaaagccacc |
| 247 | PL8 | pGL4.10- | ggcctaactggccggtaccatgacccacgtgatgctgagaagtactcctgccctagg |
| | 81 | coreCEACAM5_ | aagagactcagggcagagggaggaaggacagcagaccagacagtcacagcagccttg |
| | | 2 | acaaaacgttcctggaactaccggtgctagcctcgaggatatcaagatctggcctcg |
| | | | gcggccaagcttggcaatccggtactgttggtaaagccacc |
| 248 | PL8 | pGL4.10- | ggcctaactggccggtaccctggatgctcatcccgccaccgtcgcccaccccgccgc |
| | 82 | coreFAM111B_ | tgcagaaaggcagcaactgccacacacctaagcaacttggcgggctattcgccctgc |
| | | 1 | agctgccgccagcgcgcggctcccgccagcgcgctggcaatcaaaagtcggagaaag |
| | | | cgcgaaacctccaggcacctcccactccgcccagctaccgcgcagctcctccctagc |
| | | | ctccactgggagacaggggacgcccatgagcgggaaagagcagggcggtgattgctt |
| | | | agtttatcctgggacacgggaactggccgtggactgagtggtgccggggaggggatc |
| | | | actgagaccgggaagggtcatccagacaaatagggagggtgggcgggttggcgcgca |
| | | | gtaccctcggcccggccttcagacccacctgcgcgcgctgcgcgctcatccggtcct |
| | | | tcccttcaatcactgtctggagtgatgataattggcttccacagtggatgagagatg |
| | | | agtcatttacatccaatgagagaaaaacagcctccagagactcttcgtccattggcc |
| | | | agcgagagtgtcagttcccaggctcctgccgcgcacgggcgagcccttctaggcggg |
| | | | aaaagttcagctgagagatataaaagagcagtctttccagcacctgcaaatccagag |
| | | | cggcgggcactgacgggcacttgcaccgtgtggacagactctccggttctgtgagtg |
| | | | gtttttcttttcccgggtcggacctggagttcttagggggatggctgaaccggtgct |
| | | | agcctcgaggatatcaagatctggcctcggcggccaagcttggcaatccggtactgt |
| | | | tggtaaagccacc |
| 249 | PL8 | pGL4.10- | ggcctaactggccggtacctgagaccgggaagggtcatccagacaaatagggagggt |
| | 83 | coreFAM111B_ | gggcgggttggcgcgcagtaccctcggcccggccttcagacccacctgcgcgcgctg |
| | | 2 | cgcgctcatccggtccttcccttcaatcactgtctggagtgatgataattggcttcc |
| | | | acagtggatgagagatgagtcatttacatccaatgagagaaaaacagcctccagaga |
| | | | ctcttcgtccattggccagcgagagtgtcagttcccaggctcctgccgcgcacgggc |
| | | | gagcccttctaggcgggaaaagttcagctgagagatataaaagagcagtctttccag |
| | | | cacctgcaaatccagagcggcgggcactgacgggcacttgcaccgtgtggacagact |
| | | | ctccggttctgtgagtggtttttcttttcccgggtcggacctggagttcttaggggg |
| | | | atggctgaaccggtgctagcctcgaggatatcaagatctggcctcggcggccaagct |
| | | | tggcaatccggtactgttggtaaagccacc |
| 250 | PL8 | pGL4.10- | ggcctaactggccggtaccgggaaaagttcagctgagagatataaaagagcagtctt |
| | 84 | coreFAM111B_ | tccagcacctgcaaatccagagcggcgggcactgacgggcacttgcaccgtgtggac |
| | | 3 | agactctccggttctgtgagtggtttttcttttcccgggtcggacctggagttctta |
| | | | gggggatggctgaaccggtgctagcctcgaggatatcaagatctggcctcggcggcc |
| | | | aagcttggcaatccggtactgttggtaaagccacc |
| 251 | PL8 | pGL4.10- | ggcctaactggccggtaccctgctcctccttcttgcgggccgcgccctgccggcagt |
| | 85 | coreCEP55 | gacgtgccccgccctgcagccgcgggattcaaactcccggaagcggcatccacacct |
| | | | gatggtgtgactcggccgacgcgagcgccgcgcttcgcttcagctgctaaccggtgc |
| | | | tagcctcgaggatatcaagatctggcctcggcggccaagcttggcaatccggtactg |
| | | | ttggtaaagccacc |
| 252 | PL8 | pGL4.10- | ggcctaactggccggtaccggcccgccccctttccttacgcggattggtagctgcag |
| | 86 | coreKIF20A | gcttccctatctgattggccgaacgaacgcagcgcgtaatttaaaatattgtatctg |
| | | | taacaaagctgcacctcgtgggcggagttgtgctctgcggctgcgaaagtccagctt |
| | | | cggcgactaggtgtgagtaagccagtatcccaggaggagcaagtggcacgtcttcgg |
| | | | gtgagtgtgcggctgtgctggagcccgggttaccagctcttaccggtgctagcctcg |
| | | | aggatatcaagatctggcctcggcggccaagcttggcaatccggtactgttggtaaa |
| | | | gccacc |
| 253 | PL8 | pGL4.10- | ggcctaactggccggtaccttgttttgacaggagcagggaagtattgtagaaaataa |
| | 87 | coreAGR2_1 | tttttatcataatggagtatggcaggttatatgactgcgaggatcagaattgtgaat |
| | | | catctcttgtgtgtcttcaagtaaataaaggcaatctgcccacggagcagaaaaaaa |
| | | | atctacaaactacaaactctgtccaatcatgtaaagacaaatcagccttcaggcaaa |
| | | | tcaaatgtcttcattcaaagtctacctggatttggcactctgcccatcgtttcaaaa |
| | | | cctcttaacaatacgtttcacaaatagttaaaaacatgcatactgaaaagcatactt |
| | | | ttgcaatgttatttttaaaaacaaggaactctttaacccagggaagataatcacttg |
| | | | gggaaaggaaggttcgtttctgagttagcaacaagtaaatgcagcactagtgggtgg |
| | | | gattgaggtgtgccctggtgcataaatagagactcagctgtgctggcacactcagaa |
| | | | gcttggaccgcatcctagccgccgactcacacaaggcaggtgggtgaggaaatccag |
| | | | gtaaggctcctgacagcagctttagaagggtacttgctggagtgaattcgggcctct |
| | | | gattaccggtgctagcctcgaggatatcaagatctggcctcggcggccaagcttggc |
| | | | aatccggtactgttggtaaagccacc |
| 254 | PL8 | pGL4.10- | ggcctaactggccggtaccacctcttaacaatacgtttcacaaatagttaaaaacat |
| | 88 | coreAGR2_2 | gcatactgaaaagcatacttttgcaatgttatttttaaaaacaaggaactctttaac |
| | | | ccagggaagataatcacttggggaaaggaaggttcgtttctgagttagcaacaagta |
| | | | aatgcagcactagtggggtggattgaggtgtgccctggtgcataaatagagactcag |
| | | | ctgtgctggcacactcagaagcttggaccgcatcctagccgccgactcacacaaggc |
| | | | aggtgggtgaggaaatccaggtaaggctcctgacagcagctttagaagggtacttgc |
| | | | tggagtgaattcgggcctctgattaccggtgctagcctcgaggatatcaagatctgg |
| | | | cctcggcggccaagcttggcaatccggtactgttggtaaagccacc |
| 255 | PL8 | pGL4.10- | ggcctaactggccggtacccagtgggtaggtctagcagtggcgcagcaatagagcgc |
| | 89 | coreUBE2C | tccggagcgtctcattggctggatcaaacccaagcgagccattgattggtcgacgcc |
| | | | cccagagggttacaattcaaacgcgggcgggcgggcccgcagtcctgcagttgcagt |
| | | | cgtgttctccgagttcctgtctctctgccgagctagcctcgaggatatcaagatctg |
| | | | gcctcggcggccaagcttggcaatccggtactgttggtaaagccacc |
| 256 | PL8 | pGL4.10- | ggcctaactggccggtaccagtggtgggggagtgaaaagagagatggagaaagaggg |
| | 90 | coreCST1 | gatgggcagaaagaggaggaggagtcaggggcagggcatggaggtgggtggggctgg |
| | | | gctgccaaagcaggataaatgcacacctgcctgctggtctgggctccctgcctcggg |
| | | | ctctcaccctcctctcctgcagctccagctttgtgctctaccggtgctagcctcgag |
| | | | gatatcaagatctggcctcggcggccaagcttggcaatccggtactgttggtaaagc |
| | | | cacc |
| 257 | PL8 | hTERT- | ggcctaactggccggtaccactagtcgggttaccccacagcctaggccgattcgacc |
| | 93 | FLUC | tctctccgctggggccctcgctggcgtccctgcaccctgggagcgcgagcggcgcgc |
| | | | gggcggggaagcgcggcccagacccccgggtccgcccggagcagctgcgctgtcggg |
| | | | gccaggccgggctcccagtggattcgcgggcacagacgcccaggaccgcgcttccca |
| | | | cgtggcggagggactggggacccgggcacccgtcctgccccttcaccttccagctcc |
| | | | gcctcctccgcgcggaccccgccccgtcccgacccctcccgggtccccggcccagcc |
| | | | ccctccgggccctcccagcccctccccttcctttccgcggccccgccctctcctcgc |
| | | | ggcgcgagtttcaggcagcgctgcgtcctgctgcgcacgtgggaagccctggccccg |
| | | | gccacccccgcgatgccgcgcgctcctagctatcctcgaggatatcaagatctggcc |
| | | | tcggcggccaagcttggcaatccggtactgttggtaaagccacc |
| 258 | PL8 | pGL4.10- | ggcctaactggccggtaccctggcaggaagcctactgagatttattgaaaaggaaac |
| | 94 | murine | cgaattatcagggcactcgtttgcaacgccaacctgggctgtgttcggggcatgccc |
| | | BIRC5- | agcctgctgtctgcagtgtgaagctctttagaagccactgcaaccacaggccgcccg |
| | | FLUC | acaggaacagagacactgaaaacgggcccgcagcaaggcaggctcagcagccaacag |
| | | | tcacacccaggaagcagtatttttcttctgctcctggactctcttgcggtgtatggc |
| | | | tgcttccctttggtctgagccaggccgatggtctcagaaatagacacccattgactt |
| | | | tcttttccagcgctgggacatacagaccccgcctccatcccagggtgtctataggaa |
| | | | ggatggcggctgctgcagggaggagggtctcctgtcttcctaagggcgcccctccac |
| | | | cagcctgtgggtgggtccgaggcacttccattccgatatctagctggccaaatcctg |
| | | | caaaccttgaggcaggaagaacctgcagagcacatgggacttgcagcggacatgctt |
| | | | taaagaggtgccccaggcccgtccaccgccctcggccaccctccgtgtcctctgggg |
| | | | agcagctgcggaagattcgagtcagaatagcaagaaggaaccgcagcagaaggtaca |
| | | | actcccagcatgccctgcgcccgccacgcccacaaggccaggcgcagatgggcgtgg |
| | | | ggcgggactttcccggctcgcctcgcgccgtccactcccagaaggcagcgggcgagg |
| | | | gcgtggggccggggctctcccggcatgctctgcggcgcgcctccgcccgcgcgattt |
| | | | gaatcctgcgtttgagtcgtcttggcggaggttgtggtgacgcgctagcctcgagga |
| | | | tatcaagatctggcctcggcggccaagcttggcaatccggtactgttggtaaagcca |
| | | | cc |
| 259 | PL8 | pGL4.10- | ggcctaactggccggtaccactcccagaaggcagcgggcgagggcgtggggccgggg |
| | 95 | murine | ctctcccggcatgctctgcggcgcgcctccgcccgcgcgatttgaatcctgcgtttg |
| | | coreBIRC5- | agtcgtcttggcggaggttgtggtgacgcgctagctattctagcctcgaggatatca |
| | | FLUC | agatctggcctcggcggccaagcttggcaatccggtactgttggtaaagccacc |
| 260 | PL9 | PL- | ggcctaactggccggtaccactagtggtgactcatgggtgactcatgggtgactcat |
| | 88 | FOSL1- | gggtgactcatgggtgactcatgggtgactcatgggtgactcatgggtgactcatgg |
| | | coreCEACAM5_ | gtgactcatggtgatcatgctagcctcgaggatatcaagatcggtaccatgacccac |
| | | 2 | gtgatgctgagaagtactcctgccctaggaagagactcagggcagagggaggaagga |
| | | | cagcagaccagacagtcacagcagccttgacaaaacgttcctggaactaccggtgct |
| | | | agcctcgaggatatcaagatctggcctcggcggccaagcttggcaatccggtactgt |
| | | | tggtaaagccacc |
| 261 | PL9 | PL- | ggcctaactggccggtaccactagtggtgactcatgggtgactcatgggtgactcat |
| | 89 | FOSL1- | gggtgactcatgggtgactcatgggtgactcatgggtgactcatgggtgactcatgg |
| | | coreFAM111B_ | gtgactcatggtgatcatcgggaaaagttcagctgagagatataaaagagcagtctt |
| | | 3 | tccagcacctgcaaatccagagcggcgggcactgacgggcacttgcaccgtgtggac |
| | | | agactctccggttctgtgagtggtttttcttttcccgggtcggacctggagttctta |
| | | | gggggatggctgaaccggtgctagcctcgaggatatcaagatctggcctcggcggcc |
| | | | aagcttggcaatccggtactgttggtaaagccacc |
| 262 | PL9 | PL- | ggcctaactggccggtaccactagtggtgactcatgggtgactcatgggtgactcat |
| | 90 | FOSL1- | gggtgactcatgggtgactcatgggtgactcatgggtgactcatgggtgactcatgg |
| | | coreKIF20A | gtgactcatggtgatcatgctagcctcgaggatatcaagatcggtaccggcccgccc |
| | | | cctttccttacgcggattggtagctgcaggcttccctatctgattggccgaacgaac |
| | | | gcagcgcgtaatttaaaatattgtatctgtaacaaagctgcacctcgtgggcggagt |
| | | | tgtgctctgcggctgcgaaagtccagcttcggcgactaggtgtgagtaagccagtat |
| | | | cccaggaggagcaagtggcacgtcttcgggtgagtgtgcggctgtgctggagcccgg |
| | | | gttaccagctcttaccggtgctagcctcgaggatatcaagatctggcctcggcggcc |
| | | | aagcttggcaatccggtactgttggtaaagccacc |
| 263 | PL9 | PL- | ggcctaactggccggtaccactagtggtgactcatgggtgactcatgggtgactcat |
| | 91 | FOSL1- | gggtgactcatgggtgactcatgggtgactcatgggtgactcatgggtgactcatgg |
| | | coreCST1 | gtgactcatggtgatcatgctagcctcgaggatatcaagatcggtaccagtggtggg |
| | | | ggagtgaaaagagagatggagaaagaggggatgggcagaaagaggaggaggagtcag |
| | | | gggcagggcatggaggtgggtggggctgggctgccaaagcaggataaatgcacacct |
| | | | gcctgctggtctgggctccctgcctcgggctctcaccctcctctcctgcagctccag |
| | | | ctttgtgctctaccggtgctagcctcgaggatatcaagatctggcctcggcggccaa |
| | | | gcttggcaatccggtactgttggtaaagccacc |
| 264 | PL9 | PL- | ggcctaactggccggtaccactagtgtccccacccacacattcctgtccccacccac |
| | 92 | Canscript- | acattcctgtccccacccacacattcctgtccccacccacacattcctgtccccacc |
| | | coreCEACAM5_ | cacacattcctgtccccacccacacattcctgaccggtgctagcctcgaggatatca |
| | | 2 | agatcggtaccatgacccacgtgatgctgagaagtactcctgccctaggaagagact |
| | | | cagggcagagggaggaaggacagcagaccagacagtcacagcagccttgacaaaacg |
| | | | ttcctggaactaccggtgctagcctcgaggatatcaagatctggcctcggcggccaa |
| | | | gcttggcaatccggtactgttggtaaagccacc |
| 265 | PL9 | PL- | ggcctaactggccggtaccactagtgtccccacccacacattcctgtccccacccac |
| | 93 | Canscript- | acattcctgtccccacccacacattcctgtccccacccacacattcctgtccccacc |
| | | coreFAM111B_ | cacacattcctgtccccacccacacattcctgcgggaaaagttcagctgagagatat |
| | | 3 | aaaagagcagtctttccagcacctgcaaatccagagcggcgggcactgacgggcact |
| | | | tgcaccgtgtggacagactctccggttctgtgagtggtttttcttttcccgggtcgg |
| | | | acctggagttcttagggggatggctgaaccggtgctagcctcgaggatatcaagatc |
| | | | tggcctcggcggccaagcttggcaatccggtactgttggtaaagccacc |
| 266 | PL9 | PL- | ggcctaactggccggtaccactagtgtccccacccacacattcctgtccccacccac |
| | 94 | Canscript- | acattcctgtccccacccacacattcctgtccccacccacacattcctgtccccacc |
| | | coreKIF20A | cacacattcctgtccccacccacacattcctgcggcccgccccctttccttacgcgg |
| | | | attggtagctgcaggcttccctatctgattggccgaacgaacgcagcgcgtaattta |
| | | | aaatattgtatctgtaacaaagctgcacctcgtgggcggagttgtgctctgcggctg |
| | | | cgaaagtccagcttcggcgactaggtgtgagtaagccagtatcccaggaggagcaag |
| | | | tggcacgtcttcgggtgagtgtgcggctgtgctggagcccgggttaccagctcttac |
| | | | cggtgctagcctcgaggatatcaagatctggcctcggcggccaagcttggcaatccg |
| | | | gtactgttggtaaagccacc |
| 267 | PL9 | PL- | ggcctaactggccggtaccactagtgtccccacccacacattcctgtccccacccac |
| | 95 | Canscript- | acattcctgtccccacccacacattcctgtccccacccacacattcctgtccccacc |
| | | coreAGR2_ | cacacattcctgtccccacccacacattcctgaccggtgctagcctcgaggatatca |
| | | 2 | agatcggtaccacctcttaacaatacgtttcacaaatagttaaaaacatgcatactg |
| | | | aaaagcatacttttgcaatgttatttttaaaaacaaggaactctttaacccagggaa |
| | | | gataatcacttggggaaaggaaggttcgtttctgagttagcaacaagtaaatgcagc |
| | | | actagtgggtgggattgaggtgtgccctggtgcataaatagagactcagctgtgctg |
| | | | gcacactcagaagcttggaccgcatcctagccgccgactcacacaaggcaggtgggt |
| | | | gaggaaatccaggtaaggctcctgacagcagctttagaagggtacttgctggagtga |
| | | | attcgggcctctgattaccggtgctagcctcgaggatatcaagatctggcctcggcg |
| | | | gccaagcttggcaatccggtactgttggtaaagccacc |
| 268 | PL9 | PL- | ggcctaactggccggtaccactagtgtccccacccacacattcctgtccccacccac |
| | 96 | Canscript- | acattcctgtccccacccacacattcctgtccccacccacacattcctgtccccacc |
| | | coreCST1 | cacacattcctgtccccacccacacattcctgaccggtgctagcctcgaggatatca |
| | | | agatcggtaccagtggtgggggagtgaaaagagagatggagaaagaggggatgggca |
| | | | gaaagaggaggaggagtcaggggcagggcatggaggtgggtggggctgggctgccaa |
| | | | agcaggataaatgcacacctgcctgctggtctgggctccctgcctcgggctctcacc |
| | | | ctcctctcctgcagctccagctttgtgctctaccggtgctagcctcgaggatatcaa |
| | | | gatctggcctcggcggccaagcttggcaatccggtactgttggtaaagccacc |
| 269 | PL9 | PL- | ggcctaactggccggtaccactagtggtgactcatgggtgactcatgggtgactcat |
| | 99 | FOSL1- | gggtgactcatgggtgactcatgggtgactcatgggtgactcatgggtgactcatgg |
| | | coreAGR2_ | gtgactcatggtgatcatgctagcctcgaggatatcaagatcggtaccacctcttaa |
| | | 2 | caatacgtttcacaaatagttaaaaacatgcatactgaaaagcatacttttgcaatg |
| | | | ttatttttaaaaacaaggaactctttaacccagggaagataatcacttggggaaagg |
| | | | aaggttcgtttctgagttagcaacaagtaaatgcagcactagtgggtgggattgagg |
| | | | tgtgccctggtgcataaatagagactcagctgtgctggcacactcagaagcttggac |
| | | | cgcatcctagccgccgactcacacaaggcaggtgggtgaggaaatccaggtaaggct |
| | | | cctgacagcagctttagaagggtacttgctggagtgaattcgggcctctgattaccg |
| | | | gtgctagcctcgaggatatcaagatctggcctcggcggccaagcttggcaatccggt |
| | | | actgttggtaaagccacc |
| 271 | NP3 | NP- | aattttattgttcaaacatgagagcttagtacgtgaaacatgagagcttagtacgtt |
| | 30 | 5XFOSL1- | agccatgagagcttagtacgttagccatgagggtttagttcgttaaacatgagagct |
| | | coreBIRC5- | tagtacgttaaacatgagagcttagtacgtactatcaacaggttgaactgctgatcc |
| | | FLUC | acgttgtggtagaattggtaaagagagtcgtgtaaaatatcgagttcgcacatcttg |
| | | | ttgtctgattattgatttttggcgaaaccatttgatcatatgacaagatgtgtatct |
| | | | accttaacttaatgattttgataaaaatcattaggtacggccgcggtgccagggcgt |
| | | | gcccttgggctccccgggcgcgaCTAGTGGTGACTCATGGGTGACTCATGGGTGACT |
| | | | CATGGGTGACTCATGGGTGACTCATGtgcgctcccgacatgccccgcggcgcgccat |
| | | | taaccgccagatttgagtcgcgggacccgttggcagaggtgggaattcaccggtcga |
| | | | cgctagc |
| 273 | NP3 | NP- | aattttattgttcaaacatgagagcttagtacgtgaaacatgagagcttagtacgtt |
| | 31 | 7XFOSL1- | agccatgagagcttagtacgttagccatgagggtttagttcgttaaacatgagagct |
| | | coreBIRC5- | tagtacgttaaacatgagagcttagtacgtactatcaacaggttgaactgctgatcc |
| | | FLUC | acgttgtggtagaattggtaaagagagtcgtgtaaaatatcgagttcgcacatcttg |
| | | | ttgtctgattattgatttttggcgaaaccatttgatcatatgacaagatgtgtatct |
| | | | accttaacttaatgattttgataaaaatcattaggtacggccgcggtgccagggcgt |
| | | | gcccttgggctccccgggcgcgaCTAGTGGTGACTCATGGGTGACTCATGGGTGACT |
| | | | CATGGGTGACTCATGGGTGACTCATGGGTGACTCATGGGTGACTCATGtgcgctccc |
| | | | gacatgccccgcggcgcgccattaaccgccagatttgagtcgcgggacccgttggca |
| | | | gaggtgggaattcaccggtcgacgctagc |
| 274 | NP1 | NP- | TCTGTAGTTTGAGGAGAATATTTGTTATATTGCACAATAAAATAAGTTTGCAAGTTT |
| | 03 | AFP3- | TTTTTTTCTGCCCCAAAGAGCTCTGTGTCCTTGAACATAAAATACAAATAACCGCTA |
| | | FLUC | TGCTGTTAATTATTAACAAATGTCCCATTTTCAACCTAAGGAAATACCATAAAGTAA |
| | | | CAGATATACCAACAAAAGGTTAATAATTAACAGGCATTGCCTGAAAAGAGTATAAAA |
| | | | GGCTTTCAGCATGATTTTCCATATTGTGCTTCCACCACTGCCAATAACAAAccggtc |
| | | | gacgctagc |
| 278 | NP1 | NP-AFP- | gcccctcccccgtgccttccttgaccctggaaggtgccactcccactgtcctttcct |
| | 02 | FLUC | aataaaatgaggaaattgcatcgcattgtctgagtaggtgtcattctattctggggg |
| | | | gtggggtggggcaggacagcaagggggaggattgggaagacaatagcaggcatgctg |
| | | | gggatgcggtgggctctatggcccgggacggccgctagcccgcctaatgagcgggct |
| | | | tttttttggcttgttgtccacaaccgttaaaccttaaaagctttaaaagccttatat |
| | | | attcttttttttcttataaaacttaaaaccttagaggctatttaagttgctgattta |
| | | | tattaattttattgttcaaacatgagagcttagtacgtgaaacatgagagcttagta |
| | | | cgttagccatgagagcttagtacgttagccatgagggtttagttcgttaaacatgag |
| | | | agcttagtacgttaaacatgagagcttagtacgtactatcaacaggttgaactgctg |
| | | | atccacgttgtggtagaattggtaaagagagtcgtgtaaaatatcgagttcgcacat |
| | | | cttgttgtctgattattgatttttggcgaaaccatttgatcatatgacaagatgtgt |
| | | | atctaccttaacttaatgattttgataaaaatcattaggtacggccgcggtgccagg |
| | | | gcgtgcccttgggctccccgggcgcgaCTAGTCTCGAGTCTTGTGTGCCTGGCATAT |
| | | | GATAGGCATTTAATAGTTTTAAAGAATTAATGTATTTAGATGAATTGCATACCAAAT |
| | | | CTGCTGTCTTTTCTTTATGGCTTCATTAACTTAATTTGAGAGAAATTAATTATTCTG |
| | | | CAACTTAGGGACAAGTCATCTCTTTGAATATTCTGTAGTTTGAGGAGAATATTTGTT |
| | | | ATATTTGCAAAATAAAATAAGTTTGCAAGTTTTTTTTTTCTGCCCCAAAGAGCTCTG |
| | | | TGTCCTTGAACATAAAATACAAATAACCGCTATGCTGTTAATTATTGGCAAATGTCC |
| | | | CATTTTCAACCTAAGGAAATACCATAAAGTAACAGATATACCAACAAAAGGTTACTA |
| | | | GTTAACAGGCATTGCCTGAAAAGAGTATAAAAGAATTTCAGCATGATTTTCCATATT |
| | | | GTGCTTCCACCACTGCCAATAACAAAATAACTAGCAGAGCTAGCCtcgaggctagc |
| 279 | NP3 | NP- | aattttattgttcaaacatgagagcttagtacgtgaaacatgagagcttagtacgtt |
| | 88 | coreAGR2- | agccatgagagcttagtacgttagccatgagggtttagttcgttaaacatgagagct |
| | | FLUC | tagtacgttaaacatgagagcttagtacgtactatcaacaggttgaactgctgatcc |
| | | | acgttgtggtagaattggtaaagagagtcgtgtaaaatatcgagttcgcacatcttg |
| | | | ttgtctgattattgatttttggcgaaaccatttgatcatatgacaagatgtgtatct |
| | | | accttaacttaatgattttgataaaaatcattaggtacggccgcggtgccagggcgt |
| | | | gcccttgggctccccgggcgcgAATGCATACTAGTaacatttctctggcctaactgg |
| | | | ccggtacCACCTCTTAACAATACGTTTCACAAATAGTTAAAAACATGCATACTGAAA |
| | | | AGCATACTTTTGCAATGTTATTTTTAAAAACAAGGAACTCTTTAACCCAGGGAAGAT |
| | | | AATCACTTGGGGAAAGGAAGGTTCGTTTCTGAGTTAGCAACAAGTAAATGCAGCACT |
| | | | AGTGGGTGGGATTGAGGTgTGCCCTGGTGCATAAATAGAGACTCAGCTGTGCTGGCA |
| | | | CACTCAGAAGCTTGGACCGCATCCTAGCCGCCGACTCACACAAGGCAGGTGGGTGAG |
| | | | GAAATCCAGGTAAGGCTCCTGACAGCAGCTTTAGAAGGGTACTTGCTGGAGTGAATT |
| | | | CGGGCCTCTGATTAccggtcgacgctagc |
| 281 | NP3 | NP- | aattttattgttcaaacatgagagcttagtacgtgaaacatgagagcttagtacgtt |
| | 85 | coreCEACAM5- | agccatgagagcttagtacgttagccatgagggtttagttcgttaaacatgagagct |
| | | FLUC | tagtacgttaaacatgagagcttagtacgtactatcaacaggttgaactgctgatcc |
| | | | acgttgtggtagaattggtaaagagagtcgtgtaaaatatcgagttcgcacatcttg |
| | | | ttgtctgattattgatttttggcgaaaccatttgatcatatgacaagatgtgtatct |
| | | | accttaacttaatgattttgataaaaatcattaggtacggccgcggtgccagggcgt |
| | | | gcccttgggctccccgggcgcgAATGCATACTAGTaacatttctctggcctaactgg |
| | | | ccggtaccatgACCCACGTGATGCTGAGAAGTACTCCTGCCCTAGGAAGAGACTCAG |
| | | | GGCAGAGGGAGGAAGGACAGCAGACCAGACAGTCACAGCAGCCTTGACAAAACGTTC |
| | | | CTGGAACTaccggtcgacgctagc |
| 282 | NP3 | NP- | aattttattgttcaaacatgagagcttagtacgtgaaacatgagagcttagtacgtt |
| | 89 | coreCST- | agccatgagagcttagtacgttagccatgagggtttagttcgttaaacatgagagct |
| | | FLUC | tagtacgttaaacatgagagcttagtacgtactatcaacaggttgaactgctgatcc |
| | | | acgttgtggtagaattggtaaagagagtcgtgtaaaatatcgagttcgcacatcttg |
| | | | ttgtctgattattgatttttggcgaaaccatttgatcatatgacaagatgtgtatct |
| | | | accttaacttaatgattttgataaaaatcattaggtacggccgcggtgccagggcgt |
| | | | gcccttgggctccccgggcgcgAATGCATACTAGTaacatttctctggcctaactgg |
| | | | ccggtaccAGTGGTGGGGGAGTGAAAAGAGAGATGGAGAAAGAGGGGATGGGCAGAA |
| | | | AGAGGAGGAGGAGTCAGGGGCAGGGCATGGAGGTGGGTGGGGCTGGGCTGCCAAAGC |
| | | | AGGATAAATGCACACCTGCCTGCTGGTCTGGGCTCCCTGCCTCGGGCTCTCACCCTC |
| | | | CTCTCCTGCAGCTCCAGCTTTGTGCTCTccggtcgacgctagc |
| 283 | NP3 | NP- | aattttattgttcaaacatgagagcttagtacgtgaaacatgagagcttagtacgtt |
| | 86 | coreFAM111B- | agccatgagagcttagtacgttagccatgagggtttagttcgttaaacatgagagct |
| | | FLUC | tagtacgttaaacatgagagcttagtacgtactatcaacaggttgaactgctgatcc |
| | | | acgttgtggtagaattggtaaagagagtcgtgtaaaatatcgagttcgcacatcttg |
| | | | ttgtctgattattgatttttggcgaaaccatttgatcatatgacaagatgtgtatct |
| | | | accttaacttaatgattttgataaaaatcattaggtacggccgcggtgccagggcgt |
| | | | gcccttgggctccccgggcgcgAATGCATACTAGTaacatttctctggcctaactgg |
| | | | ccggtacCGGGAAAAGTTCAGCTGAGAGATATAAAAGAGCAGTCTTTCCAGCACCTG |
| | | | CAAATCCAGAGCGGCGGGCACTGACGGGCACTTGCACCGTGTGGACAGACTCTCCGG |
| | | | TTCTGTGAGTGGTTTTTCTTTTCCCGGGTCGGACCTGGAGTTCTTAGGGGGATGGCT |
| | | | Gaaccggtcgacgctagc |
| 284 | NP3 | NP- | aattttattgttcaaacatgagagcttagtacgtgaaacatgagagcttagtacgtt |
| | 87 | coreKIF20A- | agccatgagagcttagtacgttagccatgagggtttagttcgttaaacatgagagct |
| | | FLUC | tagtacgttaaacatgagagcttagtacgtactatcaacaggttgaactgctgatcc |
| | | | acgttgtggtagaattggtaaagagagtcgtgtaaaatatcgagttcgcacatcttg |
| | | | ttgtctgattattgatttttggcgaaaccatttgatcatatgacaagatgtgtatct |
| | | | accttaacttaatgattttgataaaaatcattaggtacggccgcggtgccagggcgt |
| | | | gcccttgggctccccgggcgcAATGCATACTAGTaacatttctctggcctaactggc |
| | | | cggtacCGGCCCGCCCCCTTTCCTTACGCGGATTGGTAGCTGCAGGCTTCCCTATCT |
| | | | GATTGGCCGAACGAACGCAGCGCGTAATTTAAAATATTGTATCTGTAACAAAGCTGC |
| | | | ACCTCGTGGGCGGAGTTGTGCTCTGCGGCTGCGAAAGTCCAGCTTCGGCGACTAGGT |
| | | | GTGAGTAAGCCAGTATCCCAGGAGGAGCAAGTGGCACGTCTTCGGGTGAGTGTGCGG |
| | | | CTGTGCTGGAGCCCGGGTTACCAGCTCTTAAccggtcgacgctagc |
| 285 | NP4 | NP- | gagagcaactgcataaggctatgaagagatacgccctggttcctggaacaattgctt |
| | 00 | CREB3L1_ | ttacagatgcacatatcgaggtggacatcacttacgctgagtacttcgaaatgtccg |
| | | v6- | ttcggttggcagaagctatgaaacgatatgggctgaatacaaatcacagaatcgtcg |
| | | coreBIRC5- | tatgcagtgaaaactctcttcaattctttatgccggtgttgggcgcgttatttatcg |
| | | FLUC | gagttgcagttgcgcccgcgaacgacatttataatgaacgtgaattgctcaacagta |
| | | | tgggcatttcgcagcctaccgtggtgttcgtttccaaaaaggggttgcaaaaaattt |
| | | | tgaacgtgcaaaaaaagctcccaatcatccaaaaaattattatcatggattctaaaa |
| | | | cggattaccagggatttcagtcgatgtacacgttcgtcacatctcatctacctcccg |
| | | | gttttaatgaatacgattttgtgccagagtccttcgatagggacaagacaattgcac |
| | | | tgatcatgaactcctctggatctactggtctgcctaaaggtgtcgctctgcctcata |
| | | | gaactgcctgcgtgagattctcgcatgccagagatcctatttttggcaatcaaatca |
| | | | ttccggatactgcgattttaagtgttgttccattccatcacggttttggaatgttta |
| | | | ctacactcggatatttgatatgtggatttcgagtcgtcttaatgtatagatttgaag |
| | | | aagagctgtttctgaggagccttcaggattacaagattcaaagtgcgctgctggtgc |
| | | | caaccctattctccttcttcgccaaaagcactctgattgacaaatacgatttatcta |
| | | | atttacacgaaattgcttctggtggcgctcccctctctaaggaagtcggggaagcgg |
| | | | ttgccaagaggttccatctgccaggtatcaggcaaggatatgggctcactgagacta |
| | | | catcagctattctgattacacccgagggggatgataaaccgggcgcggtcggtaaag |
| | | | ttgttccattttttgaagcgaaggttgtggatctggataccgggaaaacgctgggcg |
| | | | ttaatcaaagaggcgaactgtgtgtgagaggtcctatgattatgtccggttatgtaa |
| | | | acaatccggaagcgaccaacgccttgattgacaaggatggatggctacattctggag |
| | | | acatagcttactgggacgaagacgaacacttcttcatcgttgaccgcctgaagtctc |
| | | | tgattaagtacaaaggctatcaggtggctcccgctgaattggaatccatcttgctcc |
| | | | aacaccccaacatcttcgacgcaggtgtcgcaggtcttcccgacgatgacgccggtg |
| | | | aacttcccgccgccgttgttgttttggagcacggaaagacgatgacggaaaaagaga |
| | | | tcgtggattacgtcgccagtcaagtaacaaccgcgaaaaagttgcgcggaggagttg |
| | | | tgtttgtggacgaagtaccgaaaggtcttaccggaaaactcgacgcaagaaaaatca |
| | | | gagagatcctcataaaggccaagaagggcggaaagatcgccgtgtaatgaatgcatg |
| | | | aattcctgtgccttctagttgccagccatctgttgtttgcccctcccccgtgccttc |
| | | | cttgaccctggaaggtgccactcccactgtcctttcctaataaaatgaggaaattgc |
| | | | atcgcattgtctgagtaggtgtcattctattctggggggtggggtggggcaggacag |
| | | | caagggggaggattgggaagacaatagcaggcatgctggggatgcggtgggctctat |
| | | | ggcccgggacggccgctagcccgcctaatgagcgggcttttttttggcttgttgtcc |
| | | | acaaccgttaaaccttaaaagctttaaaagccttatatattcttttttttcttataa |
| | | | aacttaaaaccttagaggctatttaagttgctgatttatattaattttattgttcaa |
| | | | acatgagagcttagtacgtgaaacatgagagcttagtacgttagccatgagagctta |
| | | | gtacgttagccatgagggtttagttcgttaaacatgagagcttagtacgttaaacat |
| | | | gagagcttagtacgtactatcaacaggttgaactgctgatccacgttgtggtagaat |
| | | | tggtaaagagagtcgtgtaaaatatcgagttcgcacatcttgttgtctgattattga |
| | | | tttttggcgaaaccatttgatcatatgacaagatgtgtatctaccttaacttaatga |
| | | | ttttgataaaaatcattaggtacggccgcggtgccagggcgtgcccttgggctcccc |
| | | | gggcgcgaCTAGTAACATTTCTCTGGCCTAACTGGCCGGTACCACATCGGCTATGCT |
| | | | GCTGCTAATGCCACGTCACCACATCGACATGCCACGTCACCATCATGCCATGCCACG |
| | | | TCACCACTGCAAGATGCCACGTCACCACAGTATAATGCCACGTCACCAAGTTACTAT |
| | | | GCCACGTCACCAggtacctgcgctcccgacatgccccgcggcgcgccattaaccgcc |
| | | | agatttgagtcgcgggacccgttggcagaggtggaccggtcgacgctagc |
| 289 | NP4 | NP- | cgttgtggtagaattggtaaagagagtcgtgtaaaatatcgagttcgcacatcttgt |
| | 03 | E4AD- | tgtctgattattgatttttggcgaaaccatttgatcatatgacaagatgtgtatcta |
| | | AFP3- | ccttaacttaatgattttgataaaaatcattaggtacCACTAGTTATTAATAGTAAT |
| | | FLUC | CAATTACGGGGTCATTAGTTCATAGCCCATATATGGAGTTCCGCGTTACATAACTTA |
| | | | CGGTAAATGGCCCGCCTTGACCGCCCAACGACCCCCGCCCATTGACGTCAATAATGA |
| | | | CGTATGTTCCCATAGTAACGCCAATAGGGACTTTCCATTGACGTCAATGGGTGGAGT |
| | | | ATTTACGGTAAACTGCCCACTTGGCAGTACATCAAGTGTATCATATGCCAAGTACGC |
| | | | CCCCTATTGACGTCAATGACGGTAAATGGCCCGCCTGGCATTATGCCCAGTACATGA |
| | | | CCTTATGGGACTTTCCTACTTGGCAGTACATCTACGTATTAGTCATCGCTATTACCA |
| | | | TGGTGATGCGGTTTTGGCAGTACATCAATGGGCGTGGATAGCGGTTTGACTCACGGG |
| | | | GATTTCCAAGTCTCCACCCCATTGACGTCAATGGGAGTTTGTTTTGGCACCAAAATC |
| | | | AACGGGACTTTCCAAAATGTCGTAACAACTCCGCCCCATGGATCTCAGATTGAATTA |
| | | | TTTGCCTGTCATACAGCTAATAATTGACCATAAGACAATTAGATTTAAATTAGTTTT |
| | | | GAATCTTTCTAATACCAAAGTTCAGTTTACTGTTCCATGTTGCTTCTGAGTGGCTTC |
| | | | ACAGACTTATGAAAAAGTAAACGGAATCAGAATTACATCAATGCAAAAGCATTGCTG |
| | | | TGAACTCTGTACTTAGGACTAAACTTTGAGCAATAACACATATAGATTGAGGATTGT |
| | | | TTGCTGTTAGTATACAAACTCTGGTTCAAAGCTCCTCTTTATTGCTTGTCTTGGAAA |
| | | | ATTTGCTGTTCTTCATGGTTTCTCTTTTCACTGCTATCTATTTTTCTCAACCACTCA |
| | | | CATGGCTACAATAACTGTCTGCAAGCTTATGATTCCCAAATATCTATCTCTAGCCTC |
| | | | AATCTTGTTCCAGAAGATAAAAAGTAGTATTCAAATGCACATCAACGTCTCCACTTG |
| | | | GAGGGCTTAAAGACGTTTCAACATACAAACCGGGGAGTTTTGCCTGGAATGTTTCCT |
| | | | AAAATGTGTCCTGTAGCACATAGGGTCCTCTTGTTCCTTAAAATCTAATTACTTTTA |
| | | | GCCCAGTGCTCATCCCACCTATGGGGAGATGAGAGTGAAAAGGGAGCCTGATTAATA |
| | | | ATTACACTAAGTCAATAGGCATAGAGCCAGGACTGTTTGGGTAAACTGGTCACTTTA |
| | | | TCTTAAACTAAATATATCCAAAACTGAACATGTACTTAGTTACTAAGTCTTTGACTT |
| | | | TATCTCATTCATACCACTCAGCTTTATCCAGGCCACTTATTTGACAGTATTATTGCG |
| | | | AAAACTTCCTACTAGTGTCATCTCTTTGAATATTCTGTAGTTTGAGGAGAATATTTG |
| | | | TTATATTGCACAATAAAATAAGTTTGCAAGTTTTTTTTTTCTGCCCCAAAGAGCTCT |
| | | | GTGTCCTTGAACATAAAATACAAATAACCGCTATGCTGTTAATTATTAACAAATGTC |
| | | | CCATTTTCAACCTAAGGAAATACCATAAAGTAACAGATATACCAACAAAAGGTTAAT |
| | | | AATTAACAGGCATTGCCTGAAAAGAGTATAAAAGGCTTTCAGCATGATTTTCCATAT |
| | | | TGTGCTTCCACCACTGCCAATAACAAAccggtcgacgctagc |
| 290 | NP3 | NP- | actggtctgcctaaaggtgtcgctctgcctcatagaactgcctgcgtgagattctcg |
| | 71 | EN7R- | catgccagagatcctatttttggcaatcaaatcattccggatactgcgattttaagt |
| | | FOS- | gttgttccattccatcacggttttggaatgtttactacactcggatatttgatatgt |
| | | coreBIRC5- | ggatttcgagtcgtcttaatgtatagatttgaagaagagctgtttctgaggagcctt |
| | | FLUC | caggattacaagattcaaagtgcgctgctggtgccaaccctattctccttcttcgcc |
| | | | aaaagcactctgattgacaaatacgatttatctaatttacacgaaattgcttctggt |
| | | | ggcgctcccctctctaaggaagtcggggaagcggttgccaagaggttccatctgcca |
| | | | ggtatcaggcaaggatatgggctcactgagactacatcagctattctgattacaccc |
| | | | gagggggatgataaaccgggcgcggtcggtaaagttgttccattttttgaagcgaag |
| | | | gttgtggatctggataccgggaaaacgctgggcgttaatcaaagaggcgaactgtgt |
| | | | gtgagaggtcctatgattatgtccggttatgtaaacaatccggaagcgaccaacgcc |
| | | | ttgattgacaaggatggatggctacattctggagacatagcttactgggacgaagac |
| | | | gaacacttcttcatcgttgaccgcctgaagtctctgattaagtacaaaggctatcag |
| | | | gtggctcccgctgaattggaatccatcttgctccaacaccccaacatcttcgacgca |
| | | | ggtgtcgcaggtcttcccgacgatgacgccggtgaacttcccgccgccgttgttgtt |
| | | | ttggagcacggaaagacgatgacggaaaaagagatcgtggattacgtcgccagtcaa |
| | | | gtaacaaccgcgaaaaagttgcgcggaggagttgtgtttgtggacgaagtaccgaaa |
| | | | ggtcttaccggaaaactcgacgcaagaaaaatcagagagatcctcataaaggccaag |
| | | | aagggcggaaagatcgccgtgtaatgaatgcatgaattcctgtgccttctagttgcc |
| | | | agccatctgttgtttgcccctcccccgtgccttccttgaccctggaaggtgccactc |
| | | | ccactgtcctttcctaataaaatgaggaaattgcatcgcattgtctgagtaggtgtc |
| | | | attctattctggggggtggggtggggcaggacagcaagggggaggattgggaagaca |
| | | | atagcaggcatgctggggatgcggtgggctctatggcccgggacggccgctagcccg |
| | | | cctaatgagcgggcttttttttggcttgttgtccacaaccgttaaaccttaaaagct |
| | | | ttaaaagccttatatattcttttttttcttataaaacttaaaaccttagaggctatt |
| | | | taagttgctgatttatattaattttattgttcaaacatgagagcttagtacgtgaaa |
| | | | catgagagcttagtacgttagccatgagagcttagtacgttagccatgagggtttag |
| | | | ttcgttaaacatgagagcttagtacgttaaacatgagagcttagtacgtactatcaa |
| | | | caggttgaactgctgatccacgttgtggtagaattggtaaagagagtcgtgtaaaat |
| | | | atcgagttcgcacatcttgttgtctgattattgatttttggcgaaaccatttgatca |
| | | | tatgacaagatgtgtatctaccttaacttaatgattttgataaaaatcattaggtac |
| | | | ggccgcggtgccagggcgtgcccttgggctccccgggcgcgaCTAGTAACATTTCTC |
| | | | TGGCCTAACTGGCCGGTACCTGCCACTCAAAGTGGCACACTCCCTGCTCAGGAGGCC |
| | | | GGGAGGGAGGACACAGCCCTGGCAACTCCTCTGCCCCGGGGGGTCAGGAAGGGGTCA |
| | | | CCCCACACTCCAGAACCCTACAGAATGTGGCCTTGGCTTTTCCCATCAAGAGCTGGG |
| | | | GAAAGCCAGGCCCCGACTTCATTACCCCCTGCCCCCGTCCCATGCTCAGTGGGCCCC |
| | | | ATCGTGGGTCCATGCCACACTCCCAACTGAGCAGCCCCGCAGCCCCGCGTGTCACAG |
| | | | ACATGGGGCCTCCTAATTGCTGCTGAGGTCCCAATCCCTGGCTGGACGTGCCTGATG |
| | | | GAAGAGCCAGCTCTGGTCTCAGGGGGCTGGTTTGCAGGAGTCTCCACAGACCTGGCT |
| | | | CCAGCTTTGTGTCTTCAAATGAATACCCGGCCAAGATTGCAACTAAATTACCAGAAA |
| | | | CACTTAGGTTTCCTCACAGACTCCACAACAGGGATGGAGAAGGAAGTCAGCTGACGA |
| | | | GGTTACGACGCTGTTCGAGGGAGTCTTTCTTGGGTCACAAGTGGTAAACTGTGTTCC |
| | | | CTGAACAAAACCAGGAAGCTTTCAGTGTTTATTGTATGTACTAAGTGGAGGGAGGGG |
| | | | CTTCAGATTCTGATAAAAATATCTCCCCATTCCCAGTGCCCAATGTGACATGAATAG |
| | | | GAGGGCCCCTCCCTGAATTCCCAAGCAGATCTCCAGAGACAGCTTCAGAGAGCAGGG |
| | | | AGCCCACGGTGGCTGGGGCTTTAGGGACTTTCTGGGTTGTGGGGAGGCTAGAGGCTG |
| | | | GGCAGTCCCAGCAGGATTTGGCCTCTAGGGACCGGGCACTGTAGGGCTCAGGAGAGC |
| | | | AGCTGCCGTCCCAGTATATAAGCATAGGTGGAATTATCTGGAAACATATTTCTGCGT |
| | | | TTCACAGGCAGAGAAATCAGTCTATCCCTAAAGAATGGAAGAGCTACAGTAGCAGAC |
| | | | CTACCACCCTCCACCCTCCCACAGGCAAAAGCCCCTGAGATTCAGGTTTGGGAAGAA |
| | | | AAAGAAAATATCCCAAATATGTCATTTGAGAAAGCAGCTGCTAACCACAGGCGGCCC |
| | | | CAGCTTTTCTCAAGATCCAGGATGTGGGTTCAGTGCCCTTACTAGGGCAGTGGGGGA |
| | | | GGACGGTCAGTACCAGGACCCCAGGCACAGGCCTGGAGGACTTGCTCCCCCAAGCAA |
| | | | CTCAGATCCACGCAGAACCCATGGTACCACTAGTGGTGACTCATGGGTGACTCATGG |
| | | | GTGACTCATGGGTGACTCATGGGTGACTCATGGGTGACTCATGGGTGACTCATGGGT |
| | | | GACTCATGGGTGACTCATGtgcgctcccgacatgccccgcggcgcgccattaaccgc |
| | | | cagatttgagtcgcgggacccgttggcagaggtggaccggtcgacgctagc |
| 291 | NP3 | NP- | cgattttgtgccagagtccttcgatagggacaagacaattgcactgatcatgaactc |
| | 69 | EN18- | ctctggatctactggtctgcctaaaggtgtcgctctgcctcatagaactgcctgcgt |
| | | Canscript- | gagattctcgcatgccagagatcctatttttggcaatcaaatcattccggatactgc |
| | | FLUC | gattttaagtgttgttccattccatcacggttttggaatgtttactacactcggata |
| | | | tttgatatgtggatttcgagtcgtcttaatgtatagatttgaagaagagctgtttct |
| | | | gaggagccttcaggattacaagattcaaagtgcgctgctggtgccaaccctattctc |
| | | | cttcttcgccaaaagcactctgattgacaaatacgatttatctaatttacacgaaat |
| | | | tgcttctggtggcgctcccctctctaaggaagtcggggaagcggttgccaagaggtt |
| | | | ccatctgccaggtatcaggcaaggatatgggctcactgagactacatcagctattct |
| | | | gattacacccgagggggatgataaaccgggcgcggtcggtaaagttgttccattttt |
| | | | tgaagcgaaggttgtggatctggataccgggaaaacgctgggcgttaatcaaagagg |
| | | | cgaactgtgtgtgagaggtcctatgattatgtccggttatgtaaacaatccggaagc |
| | | | gaccaacgccttgattgacaaggatggatggctacattctggagacatagcttactg |
| | | | ggacgaagacgaacacttcttcatcgttgaccgcctgaagtctctgattaagtacaa |
| | | | aggctatcaggtggctcccgctgaattggaatccatcttgctccaacaccccaacat |
| | | | cttcgacgcaggtgtcgcaggtcttcccgacgatgacgccggtgaacttcccgccgc |
| | | | cgttgttgttttggagcacggaaagacgatgacggaaaaagagatcgtggattacgt |
| | | | cgccagtcaagtaacaaccgcgaaaaagttgcgcggaggagttgtgtttgtggacga |
| | | | agtaccgaaaggtcttaccggaaaactcgacgcaagaaaaatcagagagatcctcat |
| | | | aaaggccaagaagggcggaaagatcgccgtgtaatgaatgcatgaattcctgtgcct |
| | | | tctagttgccagccatctgttgtttgcccctcccccgtgccttccttgaccctggaa |
| | | | ggtgccactcccactgtcctttcctaataaaatgaggaaattgcatcgcattgtctg |
| | | | agtaggtgtcattctattctggggggtggggtggggcaggacagcaagggggaggat |
| | | | tgggaagacaatagcaggcatgctggggatgcggtgggctctatggcccgggacggc |
| | | | cgctagcccgcctaatgagcgggcttttttttggcttgttgtccacaaccgttaaac |
| | | | cttaaaagctttaaaagccttatatattcttttttttcttataaaacttaaaacctt |
| | | | agaggctatttaagttgctgatttatattaattttattgttcaaacatgagagctta |
| | | | gtacgtgaaacatgagagcttagtacgttagccatgagagcttagtacgttagccat |
| | | | gagggtttagttcgttaaacatgagagcttagtacgttaaacatgagagcttagtac |
| | | | gtactatcaacaggttgaactgctgatccacgttgtggtagaattggtaaagagagt |
| | | | cgtgtaaaatatcgagttcgcacatcttgttgtctgattattgatttttggcgaaac |
| | | | catttgatcatatgacaagatgtgtatctaccttaacttaatgattttgataaaaat |
| | | | cattaggtacggccgcggtgccagggcgtgcccttgggctccccgggcgcgACTAGT |
| | | | CTTCTGCCCTGAGAAAGACCTATGATTGCATGACACAAAAGAGACTGTTCAAAGGGA |
| | | | CACCATCATTCAGCAGGGCAAGCCTCCTTGCTGGGGGCAACCTGGTAGCTCCTGAGC |
| | | | CTCCCTCATCTTCACTGAGCCCCTCCAACTCTCTGAGTTCCCATGCCCCTCACTGAA |
| | | | CCTCCCTTCCCCCATGGCGAGCCTCCGCCAGCACCTTTGCACACACTCAGCCCCTTC |
| | | | CCCCTACTGAGCCCCAGCACAGTCACTGAACAGCTCTTCTTCCCCTCTGACTGAGTC |
| | | | ATCCTCCCAAGCCCTCCCCTTCCCCTCACTGAGTCTCCACCACCCCTGGTCACTGGG |
| | | | CACCCTGCTTCTGACCTCCTCCCTCCCCCAACCCCTCCACCCTTCCTCTTCACTGAG |
| | | | CCTGGCGCCTCTCACCCACCCGCCTTCCTCTCCCAGCCGCTTCTGAGCTGCCTCTTT |
| | | | GGAGCCCAACTGTCTCGCCCACGAGTCCCCATCACTCAGTCTCACTCACTCTAAGAC |
| | | | ACCTGAAAGCAGTTAGAGAACATGTGTTCATGGGGGGAGGATGAGGCTCTATCATCA |
| | | | TCCTGCAAACTAGTGTCCCCACCCACACATTCCTGTCCCCACCCACACATTCCTGTC |
| | | | CCCACCCACACATTCCTGTCCCCACCCACACATTCCTGTCCCCACCCACACATTCCT |
| | | | GTCCCCACCCACACATTCCTGAccggtcgacgctagc |
| 292 | NP3 | NP- | cgattttgtgccagagtccttcgatagggacaagacaattgcactgatcatgaactc |
| | 70 | EN19- | ctctggatctactggtctgcctaaaggtgtcgctctgcctcatagaactgcctgcgt |
| | | Canscript- | gagattctcgcatgccagagatcctatttttggcaatcaaatcattccggatactgc |
| | | FLUC | gattttaagtgttgttccattccatcacggttttggaatgtttactacactcggata |
| | | | tttgatatgtggatttcgagtcgtcttaatgtatagatttgaagaagagctgtttct |
| | | | gaggagccttcaggattacaagattcaaagtgcgctgctggtgccaaccctattctc |
| | | | cttcttcgccaaaagcactctgattgacaaatacgatttatctaatttacacgaaat |
| | | | tgcttctggtggcgctcccctctctaaggaagtcggggaagcggttgccaagaggtt |
| | | | ccatctgccaggtatcaggcaaggatatgggctcactgagactacatcagctattct |
| | | | gattacacccgagggggatgataaaccgggcgcggtcggtaaagttgttccattttt |
| | | | tgaagcgaaggttgtggatctggataccgggaaaacgctgggcgttaatcaaagagg |
| | | | cgaactgtgtgtgagaggtcctatgattatgtccggttatgtaaacaatccggaagc |
| | | | gaccaacgccttgattgacaaggatggatggctacattctggagacatagcttactg |
| | | | ggacgaagacgaacacttcttcatcgttgaccgcctgaagtctctgattaagtacaa |
| | | | aggctatcaggtggctcccgctgaattggaatccatcttgctccaacaccccaacat |
| | | | cttcgacgcaggtgtcgcaggtcttcccgacgatgacgccggtgaacttcccgccgc |
| | | | cgttgttgttttggagcacggaaagacgatgacggaaaaagagatcgtggattacgt |
| | | | cgccagtcaagtaacaaccgcgaaaaagttgcgcggaggagttgtgtttgtggacga |
| | | | agtaccgaaaggtcttaccggaaaactcgacgcaagaaaaatcagagagatcctcat |
| | | | aaaggccaagaagggcggaaagatcgccgtgtaatgaatgcatgaattcctgtgcct |
| | | | tctagttgccagccatctgttgtttgcccctcccccgtgccttccttgaccctggaa |
| | | | ggtgccactcccactgtcctttcctaataaaatgaggaaattgcatcgcattgtctg |
| | | | agtaggtgtcattctattctggggggtggggtggggcaggacagcaagggggaggat |
| | | | tgggaagacaatagcaggcatgctggggatgcggtgggctctatggcccgggacggc |
| | | | cgctagcccgcctaatgagcgggcttttttttggcttgttgtccacaaccgttaaac |
| | | | cttaaaagctttaaaagccttatatattcttttttttcttataaaacttaaaacctt |
| | | | agaggctatttaagttgctgatttatattaattttattgttcaaacatgagagctta |
| | | | gtacgtgaaacatgagagcttagtacgttagccatgagagcttagtacgttagccat |
| | | | gagggtttagttcgttaaacatgagagcttagtacgttaaacatgagagcttagtac |
| | | | gtactatcaacaggttgaactgctgatccacgttgtggtagaattggtaaagagagt |
| | | | cgtgtaaaatatcgagttcgcacatcttgttgtctgattattgatttttggcgaaac |
| | | | catttgatcatatgacaagatgtgtatctaccttaacttaatgattttgataaaaat |
| | | | cattaggtacggccgcggtgccagggcgtgcccttgggctccccgggcgcgACTAGT |
| | | | GAACATACACACCTGTGGGGGTGTCTAAGGGGCTCCCAGGGAGTTCTGGGGGGTCCT |
| | | | GGGGAGCAGGACCCTCTTCACTCCCTCCTCCAGGGGAAGTGGCCCTGGGGCACCCCA |
| | | | GGCTGTTCCCCCAGCTCTGTGGGGCCGAAGCCATCCACAGGGGGCTTTCCCCACCGG |
| | | | ATGTGGTGCGGGCCGTGGTTAATCTCACTTGAGTTAGTCACCCAGGACAAACAGCTA |
| | | | ACCGACACAATTCCTCCCAAGTCCAGGGGGCCGGAGGCGGGGTCAGCACCTGGCGGC |
| | | | AGGAGACAGTGCTGCCCTGGGATGTGGCCGGGCCTCCCTCCATTCCCAATCCTGTTG |
| | | | TCTCTGTGGCAATACCTGGCTGGGAGCTCCTATCAGGCCCGTGACCCCCGCCCTTTC |
| | | | TCCAGTGCCCTCCTGTCTGCATTCACCTGTCAGATCCCGgGGAGAGAGGGGCACTGG |
| | | | CGGCCGCCCAGGACCAGAGCTGTGGGGCCTCCCGCACCAGAGTGCAGTGAAGGTTTG |
| | | | TGGGCTGCTAGTGTCCCCACCCACACATTCCTGTCCCCACCCACACATTCCTGTCCC |
| | | | CACCCACACATTCCTGTCCCCACCCACACATTCCTGTCCCCACCCACACATTCCTGT |
| | | | CCCCACCCACACATTCCTGAccggtcgacgctagc |
| 293 | NP3 | NP- | gagagcaactgcataaggctatgaagagatacgccctggttcctggaacaattgctt |
| | 99 | ETV4- | ttacagatgcacatatcgaggtggacatcacttacgctgagtacttcgaaatgtccg |
| | | coreBIRC5- | ttcggttggcagaagctatgaaacgatatgggctgaatacaaatcacagaatcgtcg |
| | | FLUC | tatgcagtgaaaactctcttcaattctttatgccggtgttgggcgcgttatttatcg |
| | | | gagttgcagttgcgcccgcgaacgacatttataatgaacgtgaattgctcaacagta |
| | | | tgggcatttcgcagcctaccgtggtgttcgtttccaaaaaggggttgcaaaaaattt |
| | | | tgaacgtgcaaaaaaagctcccaatcatccaaaaaattattatcatggattctaaaa |
| | | | cggattaccagggatttcagtcgatgtacacgttcgtcacatctcatctacctcccg |
| | | | gttttaatgaatacgattttgtgccagagtccttcgatagggacaagacaattgcac |
| | | | tgatcatgaactcctctggatctactggtctgcctaaaggtgtcgctctgcctcata |
| | | | gaactgcctgcgtgagattctcgcatgccagagatcctatttttggcaatcaaatca |
| | | | ttccggatactgcgattttaagtgttgttccattccatcacggttttggaatgttta |
| | | | ctacactcggatatttgatatgtggatttcgagtcgtcttaatgtatagatttgaag |
| | | | aagagctgtttctgaggagccttcaggattacaagattcaaagtgcgctgctggtgc |
| | | | caaccctattctccttcttcgccaaaagcactctgattgacaaatacgatttatcta |
| | | | atttacacgaaattgcttctggtggcgctcccctctctaaggaagtcggggaagcgg |
| | | | ttgccaagaggttccatctgccaggtatcaggcaaggatatgggctcactgagacta |
| | | | catcagctattctgattacacccgagggggatgataaaccgggcgcggtcggtaaag |
| | | | ttgttccattttttgaagcgaaggttgtggatctggataccgggaaaacgctgggcg |
| | | | ttaatcaaagaggcgaactgtgtgtgagaggtcctatgattatgtccggttatgtaa |
| | | | acaatccggaagcgaccaacgccttgattgacaaggatggatggctacattctggag |
| | | | acatagcttactgggacgaagacgaacacttcttcatcgttgaccgcctgaagtctc |
| | | | tgattaagtacaaaggctatcaggtggctcccgctgaattggaatccatcttgctcc |
| | | | aacaccccaacatcttcgacgcaggtgtcgcaggtcttcccgacgatgacgccggtg |
| | | | aacttcccgccgccgttgttgttttggagcacggaaagacgatgacggaaaaagaga |
| | | | tcgtggattacgtcgccagtcaagtaacaaccgcgaaaaagttgcgcggaggagttg |
| | | | tgtttgtggacgaagtaccgaaaggtcttaccggaaaactcgacgcaagaaaaatca |
| | | | gagagatcctcataaaggccaagaagggcggaaagatcgccgtgtaatgaatgcatg |
| | | | aattcctgtgccttctagttgccagccatctgttgtttgcccctcccccgtgccttc |
| | | | cttgaccctggaaggtgccactcccactgtcctttcctaataaaatgaggaaattgc |
| | | | atcgcattgtctgagtaggtgtcattctattctggggggtggggtggggcaggacag |
| | | | caagggggaggattgggaagacaatagcaggcatgctggggatgcggtgggctctat |
| | | | ggcccgggacggccgctagcccgcctaatgagcgggcttttttttggcttgttgtcc |
| | | | acaaccgttaaaccttaaaagctttaaaagccttatatattcttttttttcttataa |
| | | | aacttaaaaccttagaggctatttaagttgctgatttatattaattttattgttcAA |
| | | | ACATGAGAGCTTAGTACGTGaaacatgagagcttagtacgtgaaacatgagagctta |
| | | | gtacgttagccatgagagcttagtacgttagccatgagggtttagttcgttaaacat |
| | | | gagagcttagtacgttaaacatgagagcttagtacgtactatcaacaggttgaactg |
| | | | ctgatccacgttgtggtagaattggtaaagagagtcgtgtaaaatatcgagttcgca |
| | | | catcttgttgtctgattattgatttttggcgaaaccatttgatcatatgacaagatg |
| | | | tgtatctaccttaacttaatgattttgataaaaatcattaggtacggccgcggtgcc |
| | | | agggcgtgcccttgggctccccgggcgcgaCTAGTAACATTTCTCTGGCCTAACTGG |
| | | | CCGGTACCACTAGTACCGGAAGTAAGAACCGGAAGTATCGACCGGAAGTAGACACCG |
| | | | GAAGTACTAACCGGAAGTAACTACCGGAAGTATGCACCGGAAGTAtgcgctcccgac |
| | | | atgccccgcggcgcgccattaaccgccagatttgagtcgcgggacccgttggcagag |
| | | | gtggaccggtcgacgctagc |
| 301 | NP3 | NP-FOS- | tcaaacatgagagcttagtacgtgaaaCATGAGAGCTTAGTACGTTAGCcatgagag |
| | 91 | coreAGR2- | cttagtacgttagccatgagagcttagtacgttagccatgagggtttagttcgttaa |
| | | FLUC | acatgagagcttagtacgttaaacatgagagcttagtacgtactatcaacaggttga |
| | | | actgctgatccacgttgtggtagaattggtaaagagagtcgtgtaaaatatcgagtt |
| | | | cgcacatcttgttgtctgattattgatttttggcgaaaccatttgatcatatgacaa |
| | | | gatgtgtatctaccttaacttaatgattttgataaaaatcattaggtacggccgcgg |
| | | | tgccagggcgtgcccttgggctccccgggcgcgaATGCATACTAGTAACATTTCTCT |
| | | | GGCCTAACTGGCCGGTACCGATCTTGATATCCTCGAGGCTAGCATGATCACCATGAG |
| | | | TCACCCATGAGTCACCCATGAGTCACCCATGAGTCACCCATGAGTCACCCATGAGTC |
| | | | ACCCATGAGTCACCCATGAGTCACCCATGAGTCACCACTAGTGGTACCACCTCTTAA |
| | | | CAATACGTTTCACAAATAGTTAAAAACATGCATACTGAAAAGCATACTTTTGCAATG |
| | | | TTATTTTTAAAAACAAGGAACTCTTTAACCCAGGGAAGATAATCACTTGGGGAAAGG |
| | | | AAGGTTCGTTTCTGAGTTAGCAACAAGTAAATGCAGCACTAGTGGGTGGGATTGAGG |
| | | | TgTGCCCTGGTGCATAAATAGAGACTCAGCTGTGCTGGCACACTCAGAAGCTTGGAC |
| | | | CGCATCCTAGCCGCCGACTCACACAAGGCAGGTGGGTGAGGAAATCCAGGTAAGGCT |
| | | | CCTGACAGCAGCTTTAGAAGGGTACTTGCTGGAGTGAATTCGGGCCTCTGATTAccg |
| | | | gtcgacgctagc |
| 302 | NP4 | NP-FOS- | aattttattgttcaaacatgagagcttagtacgtgaaacatgagagcttagtacgtt |
| | 04 | coreCEACAM- | agccatgagagcttagtacgttagccatgagggtttagttcgttaaacatgagagct |
| | | FLUC | tagtacgttaaacatgagagcttagtacgtactatcaacaggttgaactgctgatcc |
| | | | acgttgtggtagaattggtaaagagagtcgtgtaaaatatcgagttcgcacatcttg |
| | | | ttgtctgattattgatttttggcgaaaccatttgatcatatgacaagatgtgtatct |
| | | | accttaacttaatgattttgataaaaatcattaggtacggccgcggtgccagggcgt |
| | | | gcccttgggctccccgggcgcgAATGCATaCTAGTGGTGACTCATGGGTGACTCATG |
| | | | GGTGACTCATGGGTGACTCATGGGTGACTCATGGGTGACTCATGGGTGACTCATGGG |
| | | | TGACTCATGGGTGACTCATGGTGATCATGCTAGCCTCGAGGATATCAAGATCGGTAC |
| | | | CATGACCCACGTGATGCTGAGAAGTACTCCTGCCCTAGGAAGAGACTCAGGGCAGAG |
| | | | GGAGGAAGGACAGCAGACCAGACAGTCACAGCAGCCTTGACAAAACGTTCCTGGAAC |
| | | | Taccggtcgacgctagc |
| 303 | NP3 | NP-FOS- | aattttattgttcaaacatgagagcttagtacgtgaaaCATGAGAGCTTAGTACGTT |
| | 92 | coreCST- | AGCcatgagagcttagtacgttagccatgagagcttagtacgttagccatgagggtt |
| | | FLUC | tagttcgttaaacatgagagcttagtacgttaaacatgagagcttagtacgtactat |
| | | | caacaggttgaactgctgatccacgttgtggtagaattggtaaagagagtcgtgtaa |
| | | | aatatcgagttcgcacatcttgttgtctgattattgatttttggcgaaaccatttga |
| | | | tcatatgacaagatgtgtatctaccttaacttaatgattttgataaaaatcattagg |
| | | | tacggccgcggtgccagggcgtgcccttgggctccccgggcgcgAATGCATACTAGT |
| | | | AACATTTCTCTGGCCTAACTGGCCGGTACCGATCTTGATATCCTCGAGGCTAGCATG |
| | | | ATCACCATGAGTCACCCATGAGTCACCCATGAGTCACCCATGAGTCACCCATGAGTC |
| | | | ACCCATGAGTCACCCATGAGTCACCCATGAGTCACCCATGAGTCACCACTAGTGGTA |
| | | | CCAGTGGTGGGGGAGTGAAAAGAGAGATGGAGAAAGAGGGGATGGGCAGAAAGAGGA |
| | | | GGAGGAGTCAGGGGCAGGGCATGGAGGTGGGTGGGGCTGGGCTGCCAAAGCAGGATA |
| | | | AATGCACACCTGCCTGCTGGTCTGGGCTCCCTGCCTCGGGCTCTCACCCTCCTCTCC |
| | | | TGCAGCTCCAGCTTTGTGCTCTaccggtcgacgctagc |
| 304 | NP3 | NP-FOS- | aattttattgttcaaacatgagagcttagtacgtgaaacatgagagcttagtacgtt |
| | 90 | coreFAM111B- | agccatgagagcttagtacgttagccatgagggtttagttcgttaaacatgagagct |
| | | FLUC | tagtacgttaaacatgagagcttagtacgtactatcaacaggttgaactgctgatcc |
| | | | acgttgtggtagaattggtaaagagagtcgtgtaaaatatcgagttcgcacatcttg |
| | | | ttgtctgattattgatttttggcgaaaccatttgatcatatgacaagatgtgtatct |
| | | | accttaacttaatgattttgataaaaatcattaggtacggccgcggtgccagggcgt |
| | | | gcccttgggctccccgggcgcgaCTAGTAACATTTCTCTGGCCTAACTGGCCGGTAC |
| | | | CACTAGTGGTGACTCATGGGTGACTCATGGGTGACTCATGGGTGACTCATGGGTGAC |
| | | | TCATGGGTGACTCATGGGTGACTCATGGGTGACTCATGGGTGACTCATGGTGATCAT |
| | | | GCTAGCCTCGAGGATATCAAGATCGGTACCGGGAAAAGTTCAGCTGAGAGATATAAA |
| | | | AGAGCAGTCTTTCCAGCACCTGCAAATCCAGAGCGGCGGGCACTGACGGGCACTTGC |
| | | | ACCGTGTGGACAGACTCTCCGGTTCTGTGAGTGGTTTTTCTTTTCCCGGGTCGGACC |
| | | | TGGAGTTCTTAGGGGGATGGCTGaaccggtcgacgctagc |
| 305 | NP4 | NP-FOS- | ataccgggaaaacgctgggcgttaatcaaagaggcgaactgtgtgtgagaggtccta |
| | 05 | coreKIF- | tgattatgtccggttatgtaaacaatccggaagcgaccaacgccttgattgacaagg |
| | | FLUC | atggatggctacattctggagacatagcttactgggacgaagacgaacacttcttca |
| | | | tcgttgaccgcctgaagtctctgattaagtacaaaggctatcaggtggctcccgctg |
| | | | aattggaatccatcttgctccaacaccccaacatcttcgacgcaggtgtcgcaggtc |
| | | | ttcccgacgatgacgccggtgaacttcccgccgccgttgttgttttggagcacggaa |
| | | | agacgatgacggaaaaagagatcgtggattacgtcgccagtcaagtaacaaccgcga |
| | | | aaaagttgcgcggaggagttgtgtttgtggacgaagtaccgaaaggtcttaccggaa |
| | | | aactcgacgcaagaaaaatcagagagatcctcataaaggccaagaagggcggaaaga |
| | | | tcgccgtgtaatgaatgcatgaattcctgtgccttctagttgccagccatctgttgt |
| | | | ttgcccctcccccgtgccttccttgaccctggaaggtgccactcccactgtcctttc |
| | | | ctaataaaatgaggaaattgcatcgcattgtctgagtaggtgtcattctattctggg |
| | | | gggtggggtggggcaggacagcaagggggaggattgggaagacaatagcaggcatgc |
| | | | tggggatgcggtgggctctatggcccgggacggccgctagcccgcctaatgagcggg |
| | | | cttttttttggcttgttgtccacaaccgttaaaccttaaaagctttaaaagccttat |
| | | | atattcttttttttcttataaaacttaaaaccttagaggctatttaagttgctgatt |
| | | | tatattaattttattgttcaaacatgagagcttagtacgtgaaacatgagagcttag |
| | | | tacgttagccatgagagcttagtacgttagccatgagggtttagttcgttaaacatg |
| | | | agagcttagtacgttaaacatgagagcttagtacgtactatcaacaggttgaactgc |
| | | | tgatccacgttgtggtagaattggtaaagagagtcgtgtaaaatatcgagttcgcac |
| | | | atcttgttgtctgattattgatttttggcgaaaccatttgatcatatgacaagatgt |
| | | | gtatctaccttaacttaatgattttgataaaaatcattaggtacggccgcggtgcca |
| | | | gggcgtgcccttgggctccccgggcgcgAATGCATaCTAGTGGTGACTCATGGGTGA |
| | | | CTCATGGGTGACTCATGGGTGACTCATGGGTGACTCATGGGTGACTCATGGGTGACT |
| | | | CATGGGTGACTCATGGGTGACTCATGGTGATCATGCTAGCCTCGAGGATATCAAGAT |
| | | | CGGTACCGGCCCGCCCCCTTTCCTTACGCGGATTGGTAGCTGCAGGCTTCCCTATCT |
| | | | GATTGGCCGAACGAACGCAGCGCGTAATTTAAAATATTGTATCTGTAACAAAGCTGC |
| | | | ACCTCGTGGGCGGAGTTGTGCTCTGCGGCTGCGAAAGTCCAGCTTCGGCGACTAGGT |
| | | | GTGAGTAAGCCAGTATCCCAGGAGGAGCAAGTGGCACGTCTTCGGGTGAGTGTGCGG |
| | | | CTGTGCTGGAGCCCGGGTTACCAGCTCTTccggtcgacgctagc |
| 310 | NP4 | NP-FOS- | cttataaaacttaaaaccttagaggctatttaagttgctgatttatattaattttat |
| | 64 | FOS- | tgttcaaacatgagagcttagtacgtgaaaCATGAGAGCTTAGTACGTTAGCcatga |
| | | coreAGR2- | gagcttagtacgttagccatgagagcttagtacgttagccatgagggtttagttcgt |
| | | FLUC | taaacatgagagcttagtacgttaaacatgagagcttagtacgtactatcaacaggt |
| | | | tgaactgctgatccacgttgtggtagaattggtaaagagagtcgtgtaaaatatcga |
| | | | gttcgcacatcttgttgtctgattattgatttttggcgaaaccatttgatcatatga |
| | | | caagatgtgtatctaccttaacttaatgattttgataaaaatcattaggtacggccg |
| | | | cggtgccagggcgtgcccttgggctccccgggcgcgaATGCATACTAGTAACATTTC |
| | | | TCTGGCCTAACTGGCCGGTACCGATCTTGATATCCTCGAGGCTAGCATGATCACCAT |
| | | | GAGTCACCCATGAGTCACCCATGAGTCACCCATGAGTCACCCATGAGTCACCCATGA |
| | | | GTCACCCATGAGTCACCCATGAGTCACCCATGAGTCACCACTAGTGGTACCGATTCT |
| | | | TGATATCCTCGAGGCTAGCATGATCACCATGAGTCACCCATGAGTCACCCATGAGTC |
| | | | ACCCATGAGTCACCCATGAGTCACCCATGAGTCACCCATGAGTCACCCATGAGTCAC |
| | | | CCATGAGTCACCACTAGTGGTACCACCTCTTAACAATACGTTTCACAAATAGTTAAA |
| | | | AACATGCATACTGAAAAGCATACTTTTGCAATGTTATTTTTAAAAACAAGGAACTCT |
| | | | TTAACCCAGGGAAGATAATCACTTGGGGAAAGGAAGGTTCGTTTCTGAGTTAGCAAC |
| | | | AAGTAAATGCAGCACTAGTGGGTGGGATTGAGGTgTGCCCTGGTGCATAAATAGAGA |
| | | | CTCAGCTGTGCTGGCACACTCAGAAGCTTGGACCGCATCCTAGCCGCCGACTCACAC |
| | | | AAGGCAGGTGGGTGAGGAAATCCAGGTAAGGCTCCTGACAGCAGCTTTAGAAGGGTA |
| | | | CTTGCTGGAGTGAATTCGGGCCTCTGATTAccggtcgacgctagc |
| 311 | NP4 | NP-FOS- | aattttattgttcaaacatgagagcttagtacgtgaaacatgagagcttagtacgtt |
| | 06 | FOS- | agccatgagagcttagtacgttagccatgagggtttagttcgttaaacatgagagct |
| | | coreCEACAM- | tagtacgttaaacatgagagcttagtacgtactatcaacaggttgaactgctgatcc |
| | | FLUC | acgttgtggtagaattggtaaagagagtcgtgtaaaatatcgagttcgcacatcttg |
| | | | ttgtctgattattgatttttggcgaaaccatttgatcatatgacaagatgtgtatct |
| | | | accttaacttaatgattttgataaaaatcattaggtacggccgcggtgccagggcgt |
| | | | gcccttgggctccccgggcgcgAATGCATaCTAGTAACATTTCTCTGGCCTAACTGG |
| | | | CCGGTACCACTAGTGGTGACTCATGGGTGACTCATGGGTGACTCATGGGTGACTCAT |
| | | | GGGTGACTCATGGGTGACTCATGGGTGACTCATGGGTGACTCATGGGTGACTCATGG |
| | | | TGATCATGCTAGCCTCGAGGATATCAAGATCGGTACCACTAGTGGTGACTCATGGGT |
| | | | GACTCATGGGTGACTCATGGGTGACTCATGGGTGACTCATGGGTGACTCATGGGTGA |
| | | | CTCATGGGTGACTCATGGGTGACTCATGGTGATCATGCTAGCCTCGAGGATATCAAG |
| | | | ATCGGTACCATGACCCACGTGATGCTGAGAAGTACTCCTGCCCTAGGAAGAGACTCA |
| | | | GGGCAGAGGGAGGAAGGACAGCAGACCAGACAGTCACAGCAGCCTTGACAAAACGTT |
| | | | CCTGGAACTaccggtcgacgctagc |
| 312 | NP4 | NP-FOS- | cttataaaacttaaaaccttagaggctatttaagttgctgatttatattaattttat |
| | 63 | FOS- | tgttcaaacatgagagcttagtacgtgaaaCATGAGAGCTTAGTACGTTAGCcatga |
| | | FOS- | gagcttagtacgttagccatgagagcttagtacgttagccatgagggtttagttcgt |
| | | coreAGR2- | taaacatgagagcttagtacgttaaacatgagagcttagtacgtactatcaacaggt |
| | | FLUC | tgaactgctgatccacgttgtggtagaattggtaaagagagtcgtgtaaaatatcga |
| | | | gttcgcacatcttgttgtctgattattgatttttggcgaaaccatttgatcatatga |
| | | | caagatgtgtatctaccttaacttaatgattttgataaaaatcattaggtacggccg |
| | | | cggtgccagggcgtgcccttgggctccccgggcgcgaATGCATACTAGTAACATTTC |
| | | | TCTGGCCTAACTGGCCGGTACCGATCTTGATATCCTCGAGGCTAGCATGATCACCAT |
| | | | GAGTCACCCATGAGTCACCCATGAGTCACCCATGAGTCACCACTAGTGGTACCGATC |
| | | | TTGATATCCTCGAGGCTAGCATGATCACCATGAGTCACCCATGAGTCACCCATGAGT |
| | | | CACCCATGAGTCACCCATGAGTCACCCATGAGTCACCCATGAGTCACCCATGAGTCA |
| | | | CCCATGAGTCACCACTAGTGGTACCGATTCTTGATATCCTCGAGGCTAGCATGATCA |
| | | | CCATGAGTCACCCATGAGTCACCCATGAGTCACCCATGAGTCACCCATGAGTCACCC |
| | | | ATGAGTCACCCATGAGTCACCCATGAGTCACCCATGAGTCACCACTAGTGGTACCAC |
| | | | CTCTTAACAATACGTTTCACAAATAGTTAAAAACATGCATACTGAAAAGCATACTTT |
| | | | TGCAATGTTATTTTTAAAAACAAGGAACTCTTTAACCCAGGGAAGATAATCACTTGG |
| | | | GGAAAGGAAGGTTCGTTTCTGAGTTAGCAACAAGTAAATGCAGCACTAGTGGGTGGG |
| | | | ATTGAGGTgTGCCCTGGTGCATAAATAGAGACTCAGCTGTGCTGGCACACTCAGAAG |
| | | | CTTGGACCGCATCCTAGCCGCCGACTCACACAAGGCAGGTGGGTGAGGAAATCCAGG |
| | | | TAAGGCTCCTGACAGCAGCTTTAGAAGGGTACTTGCTGGAGTGAATTCGGGCCTCTG |
| | | | ATTAccggtcgacgctagc |
| 315 | NP4 | NP-FOS- | ctgggacgaagacgaacacttcttcatcgttgaccgcctgaagtctctgattaagta |
| | 59 | TATA- | caaaggctatcaggtggctcccgctgaattggaatccatcttgctccaacaccccaa |
| | | TSS- | catcttcgacgcaggtgtcgcaggtcttcccgacgatgacgccggtgaacttcccgc |
| | | FLUC- | cgccgttgttgttttggagcacggaaagacgatgacggaaaaagagatcgtggatta |
| | | 3′OIPR | cgtcgccagtcaagtaacaaccgcgaaaaagttgcgcggaggagttgtgtttgtgga |
| | | | cgaagtaccgaaaggtcttaccggaaaactcgacgcaagaaaaatcagagagatcct |
| | | | cataaaggccaagaagggcggaaagatcgccgtgtaatgaattgggATCTTCacaca |
| | | | gcagGTaaggttgcGGGCCGGGCCTGGGCCGGGTCCGGGCCGGGgcccgcctaatga |
| | | | gcgggcttttttttggcttgttgtccacaaccgttaaaccttaaaagctttaaaagc |
| | | | cttatatattcttttttttcttataaaacttaaaaccttagaggctatttaagttgc |
| | | | tgatttatattaattttattgttcaaacatgagagcttagtacgtgaaacatgagag |
| | | | cttagtacgttagccatgagagcttagtacgttagccatgagggtttagttcgttaa |
| | | | acatgagagcttagtacgttaaacatgagagcttagtacgtactatcaacaggttga |
| | | | actgctgatccacgttgtggtagaattggtaaagagagtcgtgtaaaatatcgagtt |
| | | | cgcacatcttgttgtctgattattgatttttggcgaaaccatttgatcatatgacaa |
| | | | gatgtgtatctaccttaacttaatgattttgataaaaatcattaccgcaCTGACccc |
| | | | tggtgttgcTTTTTTTTTTTAGgccgcaagCTGAAGcgtgtccctgtgccttctagt |
| | | | tgccagccatctgttgtttgcccctcccccgtgccttccttgaccctggaaggtgcc |
| | | | actcccactgtcctttcctaataaaatgaggaaattgcatcgcattgtctgagtagg |
| | | | tgtcattctattctggggggtggggtggggcaggacagcaagggggaggattgggaa |
| | | | gacaatagcaggcatgctggggatgcggtgggctctatggggtaccatgcatactag |
| | | | tGGTGACTCATGGGTGACTCATGGGTGACTCATGGGTGACTCATGGGTGACTCATGG |
| | | | GTGACTCATGGGTGACTCATGGGTGACTCATGGGTGACTCATGCGGTGCTAGCTATA |
| | | | AAAGGCCAGCAGCAGCCTGACCACATCTCATCCTCctcgaggatatcaagatctggc |
| | | | ctcggcggccagaattcaccggtcacc |
| 318 | NP3 | NP- | ggccgctagcccgcctaatgagcgggcttttttttggcttgttgtccacaaccgtta |
| | 14 | FOSL1- | aaccttaaaagctttaaaagccttatatattcttttttttcttataaaacttaaaac |
| | | Canscript- | cttagaggctatttaagttgctgatttatattaattttattgttcaaacatgagagc |
| | | coreBIRC5- | ttagtacgtgaaacatgagagcttagtacgttagccatgagagcttagtacgttagc |
| | | FLUC | catgagggtttagttcgttaaacatgagagcttagtacgttaaacatgagagcttag |
| | | | tacgtactatcaacaggttgaactgctgatccacgttgtggtagaattggtaaagag |
| | | | agtcgtgtaaaatatcgagttcgcacatcttgttgtctgattattgatttttggcga |
| | | | aaccatttgatcatatgacaagatgtgtatctaccttaacttaatgattttgataaa |
| | | | aatcattaggtacCACTAGTGGTGACTCATGGGTGACTCATGGGTGACTCATGGGTG |
| | | | ACTCATGGGTGACTCATGGGTGACTCATGGGTGACTCATGGGTGACTCATGACTAGT |
| | | | GTCCCCACCCACACATTCCTGTCCCCACCCACACATTCCTGTCCCCACCCACACATT |
| | | | CCTGTCCCCACCCACACATTCCTGTCCCCACCCACACATTCCTGTCCCCACCCACAC |
| | | | ATTCCTGtgcgctcccgacatgccccgcggcgcgccattaaccgccagatttgagtc |
| | | | gcgggacccgttggcagaggtgggctagcctcgaggatatcaagatctggcctcggc |
| | | | ggccaagcttgctagc |
| 319 | NP3 | NP- | aattttattgttcaaacatgagagcttagtacgtgaaacatgagagcttagtacgtt |
| | 08 | FOSL1- | agccatgagagcttagtacgttagccatgagggtttagttcgttaaacatgagagct |
| | | coreBIRC5- | tagtacgttaaacatgagagcttagtacgtactatcaacaggttgaactgctgatcc |
| | | FLUC | acgttgtggtagaattggtaaagagagtcgtgtaaaatatcgagttcgcacatcttg |
| | | | ttgtctgattattgatttttggcgaaaccatttgatcatatgacaagatgtgtatct |
| | | | accttaacttaatgattttgataaaaatcattaggtacggccgcggtgccagggcgt |
| | | | gcccttgggctccccgggcgcgaCTAGTGGTGACTCATGGGTGACTCATGGGTGACT |
| | | | CATGGGTGACTCATGGGTGACTCATGGGTGACTCATGGGTGACTCATGGGTGACTCA |
| | | | TGGGTGACTCATGtgcgctcccgacatgccccgcggcgcgccattaaccgccagatt |
| | | | tgagtcgcgggacccgttggcagaggtggaccggtcgacgctagc |
| 324 | NP3 | NP- | gacggccgctagcccgcctaatgagcgggcttttttttggcttgttgtccacaaccg |
| | 34 | FOSL1- | ttaaaccttaaaagctttaaaagccttatatattcttttttttcttataaaacttaa |
| | | High- | aaccttagaggctatttaagttgctgatttatattaattttattgttcAAACATGAG |
| | | FLUC | AGCTTAGTACGTGaaacatgagagcttagtacgtgaaacatgagagcttagtacgtt |
| | | | agccatgagagcttagtacgttagccatgagggtttagttcgttaaacatgagagct |
| | | | tagtacgttaaacatgagagcttagtacgtactatcaacaggttgaactgctgatcc |
| | | | acgttgtggtagaattggtaaagagagtcgtgtaaaatatcgagttcgcacatcttg |
| | | | ttgtctgattattgatttttggcgaaaccatttgatcatatgacaagatgtgtatct |
| | | | accttaacttaatgattttgataaaaatcattaggtacggccgcggtgccagggcgt |
| | | | gcccttgggctccccgggcgcgaCTAGTGGTGACTCATGGGTGACTCATGGGTGACT |
| | | | CATGGGTGACTCATGGGTGACTCATGGGTGACTCATGGGTGACTCATGGGTGACTCA |
| | | | TGGGTGACTCATGcatGGGGCGGGGtgATGACACAGCAATtcGGGACTTTCCacGCT |
| | | | TGCGTGAGAAGagACCGGAAGTgaATGACACAGCAATtcGCTTGCGTGAGAAGctGG |
| | | | GACTTTCCtaGGGGCGGGGttGGGACTTTCCacATGACACAGCAATacaAcgcGtcc |
| | | | cgacatgccccgcggcgcgccattaaccgccagatttgagtcgcgggacccgttggc |
| | | | agaggtgggaattcaccggtcgacgctagc |
| 325 | NP3 | NP- | tttattgttcaaacatgagagcttagtacgtgaaacatgagagcttagtacgttagc |
| | 32 | FOSL1- | catgagagcttagtacgttagccatgagggtttagttcgttaaacatgagagcttag |
| | | Low- | tacgttaaacatgagagcttagtacgtactatcaacaggttgaactgctgatccacg |
| | | FLUC | ttgtggtagaattggtaaagagagtcgtgtaaaatatcgagttcgcacatcttgttg |
| | | | tctgattattgatttttggcgaaaccatttgatcatatgacaagatgtgtatctacc |
| | | | ttaacttaatgattttgataaaaatcattaggtacggccgcggtgccagggcgtgcc |
| | | | cttgggctccccgggcgcgaCTAGTGGTGACTCATGGGTGACTCATGGGTGACTCAT |
| | | | GGGTGACTCATGGGTGACTCATGGGTGACTCATGGGTGACTCATGGGTGACTCATGG |
| | | | GTGACTCATGcatACCGGAAGTacTTGCGCAAtgACCGGAAGTacaAcgcGtcccga |
| | | | catgccccgcggcgcgccattaaccgccagatttgagtcgcgggacccgttggcaga |
| | | | ggtgggaattcaccggtcgacgctagc |
| 326 | NP3 | NP- | taattttattgttcaaacatgagagcttagtacgtgaaacatgagagcttagtacgt |
| | 33 | FOSL1- | tagccatgagagcttagtacgttagccatgagggtttagttcgttaaacatgagagc |
| | | Med- | ttagtacgttaaacatgagagcttagtacgtactatcaacaggttgaactgctgatc |
| | | FLUC | cacgttgtggtagaattggtaaagagagtcgtgtaaaatatcgagttcgcacatctt |
| | | | gttgtctgattattgatttttggcgaaaccatttgatcatatgacaagatgtgtatc |
| | | | taccttaacttaatgattttgataaaaatcattaggtacggccgcggtgccagggcg |
| | | | tgcccttgggctccccgggcgcgaCTAGTGGTGACTCATGGGTGACTCATGGGTGAC |
| | | | TCATGGGTGACTCATGGGTGACTCATGGGTGACTCATGGGTGACTCATGGGTGACTC |
| | | | ATGGGTGACTCATGcatTTGCGCAAcaGGGGCGGGGtgATGACACAGCAATtcGCTT |
| | | | GCGTGAGAAGagACCGGAAGTgaGGGACTTTCCacATGACACAGCAATacaAcgcGt |
| | | | cccgacatgccccgcggcgcgccattaaccgccagatttgagtcgcgggacccgttg |
| | | | gcagaggtgggaattcaccggtcgacgctagc |
| 328 | NP3 | NP- | gcccgcctaatgagcgggcttttttttggcttgttgtccacaaccgttaaaccttaa |
| | 15 | FOSL1- | aagctttaaaagccttatatattcttttttttcttataaaacttaaaaccttagagg |
| | | TATA- | ctatttaagttgctgatttatattaattttattgttcaaacatgagagcttagtacg |
| | | TSS- | tgaaacatgagagcttagtacgttagccatgagagcttagtacgttagccatgaggg |
| | | FLUC | tttagttcgttaaacatgagagcttagtacgttaaacatgagagcttagtacgtact |
| | | | atcaacaggttgaactgctgatccacgttgtggtagaattggtaaagagagtcgtgt |
| | | | aaaatatcgagttcgcacatcttgttgtctgattattgatttttggcgaaaccattt |
| | | | gatcatatgacaagatgtgtatctaccttaacttaatgattttgataaaaatcatta |
| | | | ggtacCACTAGTGGTGACTCATGGGTGACTCATGGGTGACTCATGGGTGACTCATGG |
| | | | GTGACTCATGGGTGACTCATGGGTGACTCATGGGTGACTCATGGGTGACTCATGCGG |
| | | | TGCTAGCTATAAAAGGCCAGCAGCAGCCTGACCACATCTCATCCTCctcgaggatat |
| | | | caagatctggcctcggcggccaagcttgctagc |
| 329 | NP3 | NP- | aattttattgttcaaacatgagagcttagtacgtgaaacatgagagcttagtacgtt |
| | 96 | HIGH- | agccatgagagcttagtacgttagccatgagggtttagttcgttaaacatgagagct |
| | | coreAGR2- | tagtacgttaaacatgagagcttagtacgtactatcaacaggttgaactgctgatcc |
| | | FLUC | acgttgtggtagaattggtaaagagagtcgtgtaaaatatcgagttcgcacatcttg |
| | | | ttgtctgattattgatttttggcgaaaccatttgatcatatgacaagatgtgtatct |
| | | | accttaacttaatgattttgataaaaatcattaggtacggccgcggtgccagggcgt |
| | | | gcccttgggctccccgggcgcgaCTAGTGGGGCGGGGtgATGACACAGCAATtcGGG |
| | | | ACTTTCCacGCTTGCGTGAGAAGagACCGGAAGTgaATGACACAGCAATtcGCTTGC |
| | | | GTGAGAAGctGGGACTTTCCtaGGGGCGGGGttGGGACTTTCCacATGACACAGCAA |
| | | | TacagtacCACCTCTTAACAATACGTTTCACAAATAGTTAAAAACATGCATACTGAA |
| | | | AAGCATACTTTTGCAATGTTATTTTTAAAAACAAGGAACTCTTTAACCCAGGGAAGA |
| | | | TAATCACTTGGGGAAAGGAAGGTTCGTTTCTGAGTTAGCAACAAGTAAATGCAGCAC |
| | | | TAGTGGGTGGGATTGAGGTgTGCCCTGGTGCATAAATAGAGACTCAGCTGTGCTGGC |
| | | | ACACTCAGAAGCTTGGACCGCATCCTAGCCGCCGACTCACACAAGGCAGGTGGGTGA |
| | | | GGAAATCCAGGTAAGGCTCCTGACAGCAGCTTTAGAAGGGTACTTGCTGGAGTGAAT |
| | | | TCGGGCCTCTGATTAccggtcgacgctagc |
| 330 | NP3 | NP- | GGGGCGGGGtgATGACACAGCAATtcGGGACTTTCCacGCTTGCGTGAGAAGagACC |
| | 35 | HIGH- | GGAAGTgaATGACACAGCAATtcGCTTGCGTGAGAAGctGGGACTTTCCtaGGGGCG |
| | | coreBIRC5- | GGGttGGGACTTTCCacATGACACAGCAATacaAcgcGtcccgacatgccccgcggc |
| | | FLUC | gcgccattaaccgccagatttgagtcgcgggacccgttggcagaggtgggaattcac |
| | | | cggtcgacgctagc |
| 331 | NP3 | NP- | aattttattgttcaaacatgagagcttagtacgtgaaacatgagagcttagtacgtt |
| | 93 | HIGH- | agccatgagagcttagtacgttagccatgagggtttagttcgttaaacatgagagct |
| | | coreCEACAM- | tagtacgttaaacatgagagcttagtacgtactatcaacaggttgaactgctgatcc |
| | | FLUC | acgttgtggtagaattggtaaagagagtcgtgtaaaatatcgagttcgcacatcttg |
| | | | ttgtctgattattgatttttggcgaaaccatttgatcatatgacaagatgtgtatct |
| | | | accttaacttaatgattttgataaaaatcattaggtacggccgcggtgccagggcgt |
| | | | gcccttgggctccccgggcgcgaCTAGTGGGGCGGGGtgATGACACAGCAATtcGGG |
| | | | ACTTTCCacGCTTGCGTGAGAAGagACCGGAAGTgaATGACACAGCAATtcGCTTGC |
| | | | GTGAGAAGctGGGACTTTCCtaGGGGCGGGGttGGGACTTTCCacATGACACAGCAA |
| | | | TacacTAGTAACATTTCTCTGGCCTAACTGGCCGGTACCATGACCCACGTGATGCTG |
| | | | AGAAGTACTCCTGCCCTAGGAAGAGACTCAGGGCAGAGGGAGGAAGGACAGCAGACC |
| | | | AGACAGTCACAGCAGCCTTGACAAAACGTTCCTGGAACtaccggtcgacgctagc |
| 332 | NP3 | NP- | aattttattgttcaaacatgagagcttagtacgtgaaacatgagagcttagtacgtt |
| | 97 | HIGH- | agccatgagagcttagtacgttagccatgagggtttagttcgttaaacatgagagct |
| | | coreCST- | tagtacgttaaacatgagagcttagtacgtactatcaacaggttgaactgctgatcc |
| | | FLUC | acgttgtggtagaattggtaaagagagtcgtgtaaaatatcgagttcgcacatcttg |
| | | | ttgtctgattattgatttttggcgaaaccatttgatcatatgacaagatgtgtatct |
| | | | accttaacttaatgattttgataaaaatcattaggtacggccgcggtgccagggcgt |
| | | | gcccttgggctccccgggcgcgaCTAGTGGGGCGGGGtgATGACACAGCAATtcGGG |
| | | | ACTTTCCacGCTTGCGTGAGAAGagACCGGAAGTgaATGACACAGCAATtcGCTTGC |
| | | | GTGAGAAGctGGGACTTTCCtaGGGGCGGGGttGGGACTTTCCacATGACACAGCAA |
| | | | TacactagtaacatttctctggcctaactggccggtaccAGTGGTGGGGGAGTGAAA |
| | | | AGAGAGATGGAGAAAGAGGGGATGGGCAGAAAGAGGAGGAGGAGTCAGGGGCAGGGC |
| | | | ATGGAGGTGGGTGGGGCTGGGCTGCCAAAGCAGGATAAATGCACACCTGCCTGCTGG |
| | | | TCTGGGCTCCCTGCCTCGGGCTCTCACCCTCCTCTCCTGCAGCTCCAGCTTTGTGCT |
| | | | CTaccggtcgacgctagc |
| 333 | NP3 | NP- | aattttattgttcaaacatgagagcttagtacgtgaaacatgagagcttagtacgtt |
| | 94 | HIGH- | agccatgagagcttagtacgttagccatgagggtttagttcgttaaacatgagagct |
| | | coreFAM111B- | tagtacgttaaacatgagagcttagtacgtactatcaacaggttgaactgctgatcc |
| | | FLUC | acgttgtggtagaattggtaaagagagtcgtgtaaaatatcgagttcgcacatcttg |
| | | | ttgtctgattattgatttttggcgaaaccatttgatcatatgacaagatgtgtatct |
| | | | accttaacttaatgattttgataaaaatcattaggtacggccgcggtgccagggcgt |
| | | | gcccttgggctccccgggcgcgaCTAGTGGGGCGGGGtgATGACACAGCAATtcGGG |
| | | | ACTTTCCacGCTTGCGTGAGAAGagACCGGAAGTgaATGACACAGCAATtcGCTTGC |
| | | | GTGAGAAGctGGGACTTTCCtaGGGGCGGGGttGGGACTTTCCacATGACACAGCAA |
| | | | TacacTAGTAACATTTCTCTGGCCTAACTGGCCGGTACCGGGAAAAGTTCAGCTGAG |
| | | | AGATATAAAAGAGCAGTCTTTCCAGCACCTGCAAATCCAGAGCGGCGGGCACTGACG |
| | | | GGCACTTGCACCGTGTGGACAGACTCTCCGGTTCTGTGAGTGGTTTTTCTTTTCCCG |
| | | | GGTCGGACCTGGAGTTCTTAGGGGGATGGCTGAaccggtcgacgctagc |
| 334 | NP4 | NP- | AGgccgcaagCTGAAGcgtgtccctgtgccttctagttgccagccatctgttgtttg |
| | 65 | High- | cccctcccccgtgccttccttgaccctggaaggtgccactcccactgtcctttccta |
| | | coreFAM111B- | ataaaatgaggaaattgcatcgcattgtctgagtaggtgtcattctattctgggggg |
| | | FLUC- | tggggtggggcaggacagcaagggggaggattgggaagacaatagcaggcatgctgg |
| | | 3′OIPR | ggatgcggtgggctctatggggtaccatgcataCTAGTGGGGCGGGGtgATGACACA |
| | | | GCAATtcGGGACTTTCCacGCTTGCGTGAGAAGagACCGGAAGTgaATGACACAGCA |
| | | | ATtcGCTTGCGTGAGAAGctGGGACTTTCCtaGGGGCGGGGttGGGACTTTCCacAT |
| | | | GACACAGCAATacacTAGTAACATTTCTCTGGCCTAACTGGCCGGTACCGGGAAAAG |
| | | | TTCAGCTGAGAGATATAAAAGAGCAGTCTTTCCAGCACCTGCAAATCCAGAGCGGCG |
| | | | GGCACTGACGGGCACTTGCACCGTGTGGACAGACTCTCCGGTTCTGTGAGTGGTTTT |
| | | | TCTTTTCCCGGGTCGGACCTGGAGTTCTTAGGGGGATGGCTGAagaattcaccggtc |
| | | | acc |
| 335 | NP3 | NP- | aattttattgttcaaacatgagagcttagtacgtgaaacatgagagcttagtacgtt |
| | 95 | HIGH- | agccatgagagcttagtacgttagccatgagggtttagttcgttaaacatgagagct |
| | | coreKIF20A- | tagtacgttaaacatgagagcttagtacgtactatcaacaggttgaactgctgatcc |
| | | FLUC | acgttgtggtagaattggtaaagagagtcgtgtaaaatatcgagttcgcacatcttg |
| | | | ttgtctgattattgatttttggcgaaaccatttgatcatatgacaagatgtgtatct |
| | | | accttaacttaatgattttgataaaaatcattaggtacggccgcggtgccagggcgt |
| | | | gcccttgggctccccgggcgcgaCTAGTGGGGCGGGGtgATGACACAGCAATtcGGG |
| | | | ACTTTCCacGCTTGCGTGAGAAGagACCGGAAGTgaATGACACAGCAATtcGCTTGC |
| | | | GTGAGAAGctGGGACTTTCCtaGGGGCGGGGttGGGACTTTCCacATGACACAGCAA |
| | | | TacactagtaacatttctctggcctaactggccggtacCGGCCCGCCCCCTTTCCTT |
| | | | ACGCGGATTGGTAGCTGCAGGCTTCCCTATCTGATTGGCCGAACGAACGCAGCGCGT |
| | | | AATTTAAAATATTGTATCTGTAACAAAGCTGCACCTCGTGGGCGGAGTTGTGCTCTG |
| | | | CGGCTGCGAAAGTCCAGCTTCGGCGACTAGGTGTGAGTAAGCCAGTATCCCAGGAGG |
| | | | AGCAAGTGGCACGTCTTCGGGTGAGTGTGCGGCTGTGCTGGAGCCCGGGTTACCAGC |
| | | | TCTTAaccggtcgacgctagc |
| 342 | NP4 | NP- | gagagcaactgcataaggctatgaagagatacgccctggttcctggaacaattgctt |
| | 01 | HOXA1_ | ttacagatgcacatatcgaggtggacatcacttacgctgagtacttcgaaatgtccg |
| | | v8- | ttcggttggcagaagctatgaaacgatatgggctgaatacaaatcacagaatcgtcg |
| | | coreBIRC5- | tatgcagtgaaaactctcttcaattctttatgccggtgttgggcgcgttatttatcg |
| | | FLUC | gagttgcagttgcgcccgcgaacgacatttataatgaacgtgaattgctcaacagta |
| | | | tgggcatttcgcagcctaccgtggtgttcgtttccaaaaaggggttgcaaaaaattt |
| | | | tgaacgtgcaaaaaaagctcccaatcatccaaaaaattattatcatggattctaaaa |
| | | | cggattaccagggatttcagtcgatgtacacgttcgtcacatctcatctacctcccg |
| | | | gttttaatgaatacgattttgtgccagagtccttcgatagggacaagacaattgcac |
| | | | tgatcatgaactcctctggatctactggtctgcctaaaggtgtcgctctgcctcata |
| | | | gaactgcctgcgtgagattctcgcatgccagagatcctatttttggcaatcaaatca |
| | | | ttccggatactgcgattttaagtgttgttccattccatcacggttttggaatgttta |
| | | | ctacactcggatatttgatatgtggatttcgagtcgtcttaatgtatagatttgaag |
| | | | aagagctgtttctgaggagccttcaggattacaagattcaaagtgcgctgctggtgc |
| | | | caaccctattctccttcttcgccaaaagcactctgattgacaaatacgatttatcta |
| | | | atttacacgaaattgcttctggtggcgctcccctctctaaggaagtcggggaagcgg |
| | | | ttgccaagaggttccatctgccaggtatcaggcaaggatatgggctcactgagacta |
| | | | catcagctattctgattacacccgagggggatgataaaccgggcgcggtcggtaaag |
| | | | ttgttccattttttgaagcgaaggttgtggatctggataccgggaaaacgctgggcg |
| | | | ttaatcaaagaggcgaactgtgtgtgagaggtcctatgattatgtccggttatgtaa |
| | | | acaatccggaagcgaccaacgccttgattgacaaggatggatggctacattctggag |
| | | | acatagcttactgggacgaagacgaacacttcttcatcgttgaccgcctgaagtctc |
| | | | tgattaagtacaaaggctatcaggtggctcccgctgaattggaatccatcttgctcc |
| | | | aacaccccaacatcttcgacgcaggtgtcgcaggtcttcccgacgatgacgccggtg |
| | | | aacttcccgccgccgttgttgttttggagcacggaaagacgatgacggaaaaagaga |
| | | | tcgtggattacgtcgccagtcaagtaacaaccgcgaaaaagttgcgcggaggagttg |
| | | | tgtttgtggacgaagtaccgaaaggtcttaccggaaaactcgacgcaagaaaaatca |
| | | | gagagatcctcataaaggccaagaagggcggaaagatcgccgtgtaatgaatgcatg |
| | | | aattcctgtgccttctagttgccagccatctgttgtttgcccctcccccgtgccttc |
| | | | cttgaccctggaaggtgccactcccactgtcctttcctaataaaatgaggaaattgc |
| | | | atcgcattgtctgagtaggtgtcattctattctggggggtggggtggggcaggacag |
| | | | caagggggaggattgggaagacaatagcaggcatgctggggatgcggtgggctctat |
| | | | ggcccgggacggccgctagcccgcctaatgagcgggcttttttttggcttgttgtcc |
| | | | acaaccgttaaaccttaaaagctttaaaagccttatatattcttttttttcttataa |
| | | | aacttaaaaccttagaggctatttaagttgctgatttatattaattttattgttcAA |
| | | | ACATGAGAGCTTAGTACGTGaaacatgagagcttagtacgtgaaacatgagagctta |
| | | | gtacgttagccatgagagcttagtacgttagccatgagggtttagttcgttaaacat |
| | | | gagagcttagtacgttaaacatgagagcttagtacgtactatcaacaggttgaactg |
| | | | ctgatccacgttgtggtagaattggtaaagagagtcgtgtaaaatatcgagttcgca |
| | | | catcttgttgtctgattattgatttttggcgaaaccatttgatcatatgacaagatg |
| | | | tgtatctaccttaacttaatgattttgataaaaatcattaggtacggccgcggtgcc |
| | | | agggcgtgcccttgggctccccgggcgcgaCTAGTAACATTTCTCTGGCCTAACTGG |
| | | | CCggtaccCGATGTAGCTGAGCGACAGTATAGTGCACAGTGACTGCAGCAGTCATTA |
| | | | TACGTCGCCTAAATCGAGATGCTGTACTGATCTATAAGGATCGGTAATGACGTAATG |
| | | | ACGTAATGACGTAATGACGTAATGACGTAATGAcggtacctgcgctcccgacatgcc |
| | | | ccgcggcgcgccattaaccgccagatttgagtcgcgggacccgttggcagaggtgga |
| | | | ccggtcgacgctagc |
| 343 | NP4 | NP- | aactgcataaggctatgaagagatacgccctggttcctggaacaattgcttttacag |
| | 02 | HOXC10_ | atgcacatatcgaggtggacatcacttacgctgagtacttcgaaatgtccgttcggt |
| | | v24- | tggcagaagctatgaaacgatatgggctgaatacaaatcacagaatcgtcgtatgca |
| | | coreBIRC5- | gtgaaaactctcttcaattctttatgccggtgttgggcgcgttatttatcggagttg |
| | | FLUC | cagttgcgcccgcgaacgacatttataatgaacgtgaattgctcaacagtatgggca |
| | | | tttcgcagcctaccgtggtgttcgtttccaaaaaggggttgcaaaaaattttgaacg |
| | | | tgcaaaaaaagctcccaatcatccaaaaaattattatcatggattctaaaacggatt |
| | | | accagggatttcagtcgatgtacacgttcgtcacatctcatctacctcccggtttta |
| | | | atgaatacgattttgtgccagagtccttcgatagggacaagacaattgcactgatca |
| | | | tgaactcctctggatctactggtctgcctaaaggtgtcgctctgcctcatagaactg |
| | | | cctgcgtgagattctcgcatgccagagatcctatttttggcaatcaaatcattccgg |
| | | | atactgcgattttaagtgttgttccattccatcacggttttggaatgtttactacac |
| | | | tcggatatttgatatgtggatttcgagtcgtcttaatgtatagatttgaagaagagc |
| | | | tgtttctgaggagccttcaggattacaagattcaaagtgcgctgctggtgccaaccc |
| | | | tattctccttcttcgccaaaagcactctgattgacaaatacgatttatctaatttac |
| | | | acgaaattgcttctggtggcgctcccctctctaaggaagtcggggaagcggttgcca |
| | | | agaggttccatctgccaggtatcaggcaaggatatgggctcactgagactacatcag |
| | | | ctattctgattacacccgagggggatgataaaccgggcgcggtcggtaaagttgttc |
| | | | cattttttgaagcgaaggttgtggatctggataccgggaaaacgctgggcgttaatc |
| | | | aaagaggcgaactgtgtgtgagaggtcctatgattatgtccggttatgtaaacaatc |
| | | | cggaagcgaccaacgccttgattgacaaggatggatggctacattctggagacatag |
| | | | cttactgggacgaagacgaacacttcttcatcgttgaccgcctgaagtctctgatta |
| | | | agtacaaaggctatcaggtggctcccgctgaattggaatccatcttgctccaacacc |
| | | | ccaacatcttcgacgcaggtgtcgcaggtcttcccgacgatgacgccggtgaacttc |
| | | | ccgccgccgttgttgttttggagcacggaaagacgatgacggaaaaagagatcgtgg |
| | | | attacgtcgccagtcaagtaacaaccgcgaaaaagttgcgcggaggagttgtgtttg |
| | | | tggacgaagtaccgaaaggtcttaccggaaaactcgacgcaagaaaaatcagagaga |
| | | | tcctcataaaggccaagaagggcggaaagatcgccgtgtaatgaatgcatgaattcc |
| | | | tgtgccttctagttgccagccatctgttgtttgcccctcccccgtgccttccttgac |
| | | | cctggaaggtgccactcccactgtcctttcctaataaaatgaggaaattgcatcgca |
| | | | ttgtctgagtaggtgtcattctattctggggggtggggtggggcaggacagcaaggg |
| | | | ggaggattgggaagacaatagcaggcatgctggggatgcggtgggctctatggcccg |
| | | | ggacggccgctagcccgcctaatgagcgggcttttttttggcttgttgtccacaacc |
| | | | gttaaaccttaaaagctttaaaagccttatatattcttttttttcttataaaactta |
| | | | aaaccttagaggctatttaagttgctgatttatattaattttattgttcaaacatga |
| | | | gagcttagtacgtgaaaCATGAGAGCTTAGTACGTTAGCcatgagagcttagtacgt |
| | | | tagccatgagagcttagtacgttagccatgagggtttagttcgttaaacatgagagc |
| | | | ttagtacgttaaacatgagagcttagtacgtactatcaacaggttgaactgctgatc |
| | | | cacgttgtggtagaattggtaaagagagtcgtgtaaaatatcgagttcgcacatctt |
| | | | gttgtctgattattgatttttggcgaaaccatttgatcatatgacaagatgtgtatc |
| | | | taccttaacttaatgattttgataaaaatcattaggtacggccgcggtgccagggcg |
| | | | tgcccttgggctccccgggcgcgaCTAGTAACATTTCTCTGGCCTAACTGGCCggta |
| | | | CCAGCTGAGCGACAGTATAGTGCACAGTGACTGCAGCAGTCATTATACGTCGCCTAA |
| | | | ATCGAGATGCTGTACTGATCTATAAGTCGTAAACTGTCGTAAACTGTCGTAAACTGT |
| | | | CGTAAACTGTCGTAAACTGTCGTAAACTggtacctgcgctcccgacatgccccgcgg |
| | | | cgcgccattaaccgccagatttgagtcgcgggacccgttggcagaggtggaccggtc |
| | | | gacgctagc |

TABLE 1
Sequences of Synthetic Response Elements
(SREs) according to the disclosure

| SEQ | | |
|---|---|---|
| ID | | |
| NO: | Name | Sequence |
| 377 | SRE001 | Cggagtactgtcctccgagcggagtactgtcctccgagcggagtactgtcctccgagcgga |
| | | gtactgtcctccgagcggagtactgtcctccgag |
| 378 | SRE002 | GGTGACTCATGGGTGACTCATGGGTGACTCATGGGTGACTCATGGGTGACTCATGGGTGAC |
| | | TCATGGGTGACTCATGGGTGACTCATGGGTGACTCATG |
| 379 | SRE003 | GGGGCGGGGtgATGACACAGCAATtcGGGACTTTCCacGCTTGCGTGAGAAGagACCGGAA |
| | | GTgaATGACACAGCAATtcGCTTGCGTGAGAAGctGGGACTTTCCtaGGGGCGGGGttGGG |
| | | ACTTTCCacATGACACAGCAATac |
| 380 | SRE004 | AATAGGTACCACTAGTGTCCCCACCCACACATTCCTGTCCCCACCCACACATTCCTGTCCC |
| | | CACCCACACATTCCTGTCCCCACCCACACATTCCTGTCCCCACCCACACATTCCTGTCCCC |
| | | ACCCACACATTCCTGACCGGTGctagcctcgag |
| 381 | SRE005 | CTGAGCGACAGTATAGTGCACAGTGACTGCAGCAGTCATTCCTTTGATGTACGCAACTCCT |
| | | TTGATGTCTATGCGTCCTTTGATGTTAAGGATTCCTTTGATGTAGGTACATCCTTTGATGT |
| | | CCGTAAATCCTTTGATGTGACgatcttgatatc |
| 382 | SRE006 | TACCTGATCAAACATGCCCGGACATGTCGTAAGACATAAACATGCCCGGACATGTCCTCGC |
| | | AATCTAACATGCCCGGACATGTCCTCGCAATCTAACATGCCCGGACATGTCTGCAAGCTAC |
| | | AACATGCCCGGACATGTC |
| 383 | SRE007 | GGGGCGGGGTGATGACACAGCAATTCGGGACTTTCCACGCTTGCGTGAGAAGAGACCGGAA |
| | | GTGAATGACACAGCAAT |
| 384 | SRE008 | GCTTGCGTGAGAAGctGGGACTTTCCtaGGGGCGGGGttGGGACTTTCCacATGACACAGC |
| | | AATac |
| 385 | SRE009 | GGTGACTCATGGGTGACTCATGGGTGACTCATGCTaCgTgTgAcGGTGACTCATGGGTGAC |
| | | TCATGGGTGACTCATGaagTcgcaGattGGTGACTCATGGGTGACTCATGGGTGACTCATG |
| 386 | SRE010 | GGTGACTCATGATGATGCCACGTCACCAATGCCACGTCACCAGGTGACTCATGGGTGACTC |
| | | ATGACGTGTGACATGCCACGTCACCAATGCCACGTCACCAGGTGACTCATGGGTGACTCAT |
| | | G |
| 387 | SRE011 | GGGAGGAAGTCGTAAAACTTGGGAGGAAGTCGTAAAAAATGGGAGGAAGTCGTAAAATGCG |
| | | GGAGGAAGTCGTAAAAGAAGGGAGGAAGTCGTAAAAATCGGGAGGAAGTCGTAAAA |
| 388 | SRE012 | ATGACTCAGCAATTAGCGAGTTAGAATGACTCAGCAATTATGCGTCGGACATGACTCAGCA |
| | | ATTACATCTCGATTATGACTCAGCAATTAGGATAGGCATATGACTCAGCAATTACATAGCA |
| | | GCAATGACTCAGCAATTA |
| 389 | SRE013 | ACATCAAAGGATTTACGGACATCAAAGGATGTACCTACATCAAAGGAATCCTTAACATCAA |
| | | AGGACGCATAGACATCAAAGGAGTTGCGTACATCAAAGGA |
| 390 | SRE014 | CACTTCCGGTTTACTTCCACTTCCGGTTTACTAGCACTTCCGGTTTACGCTCACTTCCGGT |
| | | TTACGATCACTTCCGGTTTACAGACACTTCCGGTTTAC |
| 391 | SRE015 | GCGTCCGCCCGAGTCCCCGCCTCGCCGCCAACGCCAAtgcTcatGCGTCCGCCCGAGTCCC |
| | | CGCCTCGCCGCCAACGCCAtcatgcctGCGTCCGCCCGAGTCCCCGCCTCGCCGCCAACGC |
| | | CA |
| 392 | SRE016 | CAACATGGCGGCGCCCAACATGGCGGCTACCAACATGGCGGCCTCCAACATGGCGGCAGGC |
| | | AACATGGCGGCTGCCAACATGGCGGC |
| 393 | SRE017 | TGGTTGCTGACTAATTGAGATGCATGCTTTGCATACTTCTGCCTGCTGGGGAGCCTGGGGA |
| | | CTTTCCACAC |
| 394 | SRE018 | GCTCACTCACTCACTCACTGAGGCCTGCAGAGCAAAGCTCTGCAGTCTGGGGACCTTTGGT |
| | | CCCCAGGCCTCAGTGAGTGAGTGAGTGAGCAGAGAGGGAGTGGCCAACTCCATCACTAGGG |
| | | GTTCCT |
| 395 | SRE019 | GGTGACTCATGGGTGACTCATGGGTGACTCATGGGTGACTCATGCTaCgTGGTGACTCATG |
| | | GGTGACTCATGGGTGACTCATGGGTGACTCATGGGTGACTCATG |
| 396 | SRE020 | AGTATAGTGCACAGTGACTGCAGCAGGGTGACTCATGATGATGCCACGTCACCAATGCCAC |
| | | GTCACCAGGTGACTCATGGGTGACTCATGATGCCACGTCACCAATGCCACGTCACCAGGTG |
| | | ACTCATGGGTGACTCATG |
| 397 | SRE021 | TAATTGCTGAGTCATTGCTGCTATGTAATTGCTGAGTCATATGCCTATCCTCCTTTGATGT |
| | | ACGCAACTCCTTTGATGTCTATGCGTAATTGCTGAGTCATAATCGAGATGTAATTGCTGAG |
| | | TCATGTCCGACGCATCCTTTGATGTTAAGGATTCCTTTGATGTAGGTACATAATTGCTGAG |
| | | TCATTCTAACTCGCTAATTGCTGAGTCATCATCTCGACCTCCTTTGATGTCCGTAAATCCT |
| | | TTGATGT |

TABLE 1C
Sequences of Synthetic Response Sensors (SRSs)
according to the disclosure

| SEQ | | |
|---|---|---|
| ID | | |
| NO: | Name | Sequence |
| 398 | SRS002 | ACTAGTGGTGACTCATGGGTGACTCATGGGTGACTCATGGGTGACTCATGGGTGACTCATG |
| | | GGTGACTCATGGGTGACTCATGGGTGACTCATGGGTGACTCATGtgcgctcccgacatgcc |
| | | ccgcggcgcgccattaaccgccagatttgagtcgcgggacccgttggcagaggtgg |
| 399 | SRS003 | agcttgcatgcctgcaggtcggagtactgtcctccgagcggagtactgtcctccgagcgga |
| | | gtactgtcctccgagcggagtactgtcctccgagcggagtactgtcctccgagcggtgcgc |
| | | tcccgacatgccccgcggcgcgccattaaccgccagatttgagtcgcgggacccgttggca |
| | | gaggtggg |
| 400 | SRS004 | ctcgaggctagcATGATCACCATGAGTCACCCATGAGTCACCCATGAGTCACCCATGAGTC |
| | | ACCCATGAGTCACCCATGAGTCACCCATGAGTCACCCATGAGTCACCCATGAGTCACCACT |
| | | AGTGGTACCACCTCTTAACAATACGTTTCACAAATAGTTAAAAACATGCATACTGAAAAGC |
| | | ATACTTTTGCAATGTTATTTTTAAAAACAAGGAACTCTTTAACCCAGGGAAGATAATCACT |
| | | TGGGGAAAGGAAGGTTCGTTTCTGAGTTAGCAACAAGTAAATGCAGCACTAGTGGGTGGGA |
| | | TTGAGGTgTGCCCTGGTGCATAAATAGAGACTCAGCTGTGCTGGCACACTCAGAAGCTTGG |
| | | ACCGCATCCTAGCCGCCGACTCACACAAGGCAGGTGGGTGAGGAAATCCAGGTAAGGCTCC |
| | | TGACAGCAGCTTTAGAAGGGTACTTGCTGGAGTGAATTCGGGCCTCTGATTA |
| 401 | SRS005 | GGTGACTCATGGGTGACTCATGGGTGACTCATGGGTGACTCATGGGTGACTCATGGGTGAC |
| | | TCATGGGTGACTCATGGGTGACTCATGGGTGACTCATGGTGATCATGCTAGCCTCGAGGAT |
| | | ATCAAGATCGGTACCGGGAAAAGTTCAGCTGAGAGATATAAAAGAGCAGTCTTTCCAGCAC |
| | | CTGCAAATCCAGAGCGGCGGGCACTGACGGGCACTTGCACCGTGTGGACAGACTCTCCGGT |
| | | TCTGTGAGTGGTTTTTCTTTTCCCGGGTCGGACCTGGAGTTCTTAGGGGGATGGCTGa |
| 402 | SRS006 | GGTGACTCATGGGTGACTCATGGGTGACTCATGGGTGACTCATGGGTGACTCATGGGTGAC |
| | | TCATGGGTGACTCATGGGTGACTCATGGGTGACTCATGGTGATCATGCTAGCCTCGAGGAT |
| | | ATCAAGATCGGTACCATGACCCACGTGATGCTGAGAAGTACTCCTGCCCTAGGAAGAGACT |
| | | CAGGGCAGAGGGAGGAAGGACAGCAGACCAGACAGTCACAGCAGCCTTGACAAAACGTTCC |
| | | TGGAACT |
| 403 | SRS007 | GGTGACTCATGGGTGACTCATGGGTGACTCATGGGTGACTCATGGGTGACTCATGGGTGAC |
| | | TCATGGGTGACTCATGGGTGACTCATGGGTGACTCATGGTGATCATGCTAGCCTCGAGGAT |
| | | ATCAAGATCGGTACCGGCCCGCCCCCTTTCCTTACGCGGATTGGTAGCTGCAGGCTTCCCT |
| | | ATCTGATTGGCCGAACGAACGCAGCGCGTAATTTAAAATATTGTATCTGTAACAAAGCTGC |
| | | ACCTCGTGGGCGGAGTTGTGCTCTGCGGCTGCGAAAGTCCAGCTTCGGCGACTAGGTGTGA |
| | | GTAAGCCAGTATCCCAGGAGGAGCAAGTGGCACGTCTTCGGGTGAGTGTGCGGCTGTGCTG |
| | | GAGCCCGGGTTACCAGCTCTT |
| 404 | SRS008 | GGTGACTCATGGGTGACTCATGGGTGACTCATGGGTGACTCATGGGTGACTCATGGGTGAC |
| | | TCATGGGTGACTCATGGGTGACTCATGGGTGACTCATGCGGTGCTAGCTATAAAAGGCCAG |
| | | CAGCAGCCTGACCACATCTCATCCTCctcgaggatatcaagatctggcctcggcggccaaa |
| | | ttca |
| 405 | SRS009 | GGGGCGGGGtgATGACACAGCAATtcGGGACTTTCCacGCTTGCGTGAGAAGagACCGGAA |
| | | GTgaATGACACAGCAATtcGCTTGCGTGAGAAGctGGGACTTTCCtaGGGGCGGGGttGGG |
| | | ACTTTCCacATGACACAGCAATacaAcgcGtcccgacatgccccgcggcgcgccattaacc |
| | | gccagatttgagtcgcgggacccgttggcagaggtgg |
| 406 | SRS010 | GGGGCGGGGtgATGACACAGCAATtcGGGACTTTCCacGCTTGCGTGAGAAGagACCGGAA |
| | | GTgaATGACACAGCAATtcGCTTGCGTGAGAAGctGGGACTTTCCtaGGGGCGGGGttGGG |
| | | ACTTTCCacATGACACAGCAATacagtacCACCTCTTAACAATACGTTTCACAAATAGTTA |
| | | AAAACATGCATACTGAAAAGCATACTTTTGCAATGTTATTTTTAAAAACAAGGAACTCTTT |
| | | AACCCAGGGAAGATAATCACTTGGGGAAAGGAAGGTTCGTTTCTGAGTTAGCAACAAGTAA |
| | | ATGCAGCACTAGTGGGTGGGATTGAGGTgTGCCCTGGTGCATAAATAGAGACTCAGCTGTG |
| | | CTGGCACACTCAGAAGCTTGGACCGCATCCTAGCCGCCGACTCACACAAGGCAGGTGGGTG |
| | | AGGAAATCCAGGTAAGGCTCCTGACAGCAGCTTTAGAAGGGTACTTGCTGGAGTGAATTCG |
| | | GGCCTCTGATT |
| 407 | SRS011 | GGGGCGGGGtgATGACACAGCAATtcGGGACTTTCCacGCTTGCGTGAGAAGagACCGGAA |
| | | GTgaATGACACAGCAATtcGCTTGCGTGAGAAGctGGGACTTTCCtaGGGGCGGGGttGGG |
| | | ACTTTCCacATGACACAGCAATacacTAGTAACATTTCTCTGGCCTAACTGGCCGGTACCG |
| | | GGAAAAGTTCAGCTGAGAGATATAAAAGAGCAGTCTTTCCAGCACCTGCAAATCCAGAGCG |
| | | GCGGGCACTGACGGGCACTTGCACCGTGTGGACAGACTCTCCGGTTCTGTGAGTGGTTTTT |
| | | CTTTTCCCGGGTCGGACCTGGAGTTCTTAGGGGGATGGCTGA |
| 408 | SRS012 | GGGGCGGGGtgATGACACAGCAATtcGGGACTTTCCacGCTTGCGTGAGAAGagACCGGAA |
| | | GTgaATGACACAGCAATtcGCTTGCGTGAGAAGctGGGACTTTCCtaGGGGCGGGGttGGG |
| | | ACTTTCCacATGACACAGCAATacacTAGTAACATTTCTCTGGCCTAACTGGCCGGTACCA |
| | | TGACCCACGTGATGCTGAGAAGTACTCCTGCCCTAGGAAGAGACTCAGGGCAGAGGGAGGA |
| | | AGGACAGCAGACCAGACAGTCACAGCAGCCTTGACAAAACGTTCCTGGAACt |
| 409 | SRS013 | GGGGCGGGGtgATGACACAGCAATtcGGGACTTTCCacGCTTGCGTGAGAAGagACCGGAA |
| | | GTgaATGACACAGCAATtcGCTTGCGTGAGAAGctGGGACTTTCCtaGGGGCGGGGttGGG |
| | | ACTTTCCacATGACACAGCAATacactagtaacatttctctggcctaactggccggtacCG |
| | | GCCCGCCCCCTTTCCTTACGCGGATTGGTAGCTGCAGGCTTCCCTATCTGATTGGCCGAAC |
| | | GAACGCAGCGCGTAATTTAAAATATTGTATCTGTAACAAAGCTGCACCTCGTGGGCGGAGT |
| | | TGTGCTCTGCGGCTGCGAAAGTCCAGCTTCGGCGACTAGGTGTGAGTAAGCCAGTATCCCA |
| | | GGAGGAGCAAGTGGCACGTCTTCGGGTGAGTGTGCGGCTGTGCTGGAGCCCGGGTTACCAG |
| | | CTCTTA |
| 410 | SRS014 | TCCCCACCCACACATTCCTGTCCCCACCCACACATTCCTGTCCCCACCCACACATTCCTGT |
| | | CCCCACCCACACATTCCTGTCCCCACCCACACATTCCTGTCCCCACCCACACATTCCTGtg |
| | | cgctcccgacatgccccgcggcgcgccattaaccgccagatttgagtcgcgggacccgttg |
| | | gcagaggtgg |
| 411 | SRS015 | GGTGACTCATGGGTGACTCATGGGTGACTCATGGGTGACTCATGGGTGACTCATGGGTGAC |
| | | TCATGGGTGACTCATGGGTGACTCATGACTAGTGTCCCCACCCACACATTCCTGTCCCCAC |
| | | CCACACATTCCTGTCCCCACCCACACATTCCTGTCCCCACCCACACATTCCTGTCCCCACC |
| | | CACACATTCCTGTCCCCACCCACACATTCCTGtgcgctcccgacatgccccgcggcgcgcc |
| | | attaaccgccagatttgagtcgcgggacccgttggcagaggtgg |
| 412 | SRS016 | CTGAGCGACAGTATAGTGCACAGTGACTGCAGCAGTCATTCCTTTGATGTACGCAACTCCT |
| | | TTGATGTCTATGCGTCCTTTGATGTTAAGGATTCCTTTGATGTAGGTACATCCTTTGATGT |
| | | CCGTAAATCCTTTGATGTGACGTCTACGTACATACTGAAAAGCATACTTTTGCAATGTTAT |
| | | TTTTAAAAACAAGGAACTCTTTAACCCAGGGAAGATAATCACTTGGGGAAAGGAAGGTTCG |
| | | TTTCTGAGTTAGCAACAAGTAAATGCAGCACTAGTGGGTGGGATTGAGGTgTGCCCTGGTG |
| | | CATAAATAGAGACTCAGCTGTGCTGGCACACTCAGAAGCTTGGACCGCATCCTAGCCGCCG |
| | | ACTCACACAAGGCAGGTGGGTGAGGAAATCCAGGTAAGGCTCCTGACAGCAGCTTTAGAAG |
| | | GGTACTTGCTGGAGTGAATTCGGGCCTCTGATTA |
| 413 | SRS017 | CTGAGCGACAGTATAGTGCACAGTGACTGCAGCAGTCATTCCTTTGATGTACGCAACTCCT |
| | | TTGATGTCTATGCGTCCTTTGATGTTAAGGATTCCTTTGATGTAGGTACATCCTTTGATGT |
| | | CCGTAAATCCTTTGATGTGACgatcttgatatcctcgaggctagcATGATCACCATGAGTC |
| | | ACCCATGAGTCACCCATGAGTCACCCATGAGTCACCCATGAGTCACCCATGAGTCACCCAT |
| | | GAGTCACCCATGAGTCACCCATGAGTCACCACTAGTGGTACCACCTCTTAACAATACGTTT |
| | | CACAAATAGTTAAAAACATGCATACTGAAAAGCATACTTTTGCAATGTTATTTTTAAAAAC |
| | | AAGGAACTCTTTAACCCAGGGAAGATAATCACTTGGGGAAAGGAAGGTTCGTTTCTGAGTT |
| | | AGCAACAAGTAAATGCAGCACTAGTGGGTGGGATTGAGGTgTGCCCTGGTGCATAAATAGA |
| | | GACTCAGCTGTGCTGGCACACTCAGAAGCTTGGACCGCATCCTAGCCGCCGACTCACACAA |
| | | GGCAGGTGGGTGAGGAAATCCAGGTAAGGCTCCTGACAGCAGCTTTAGAAGGGTACTTGCT |
| | | GGAGTGAATTCGGGCCTCTGATTA |
| 414 | SRS018 | CTGAGCGACAGTATAGTGCACAGTGACTGCAGCAGTCATTCCTTTGATGTACGCAACTCCT |
| | | TTGATGTCTATGCGTCCTTTGATGTTAAGGATTCCTTTGATGTAGGTACATCCTTTGATGT |
| | | CCGTAAATCCTTTGATGTGACGTCTACGTATCTACCTGATCAAACATGCCCGGACATGTCG |
| | | TAAGACATAAACATGCCCGGACATGTCCTCGCAATCTAACATGCCCGGACATGTCCTCGCA |
| | | ATCTAACATGCCCGGACATGTCTGCAAGCTACAACATGCCCGGACATGTCTACAATATACG |
| | | TATCTACCTGATCAAACATGCCCGGACATGTCGTAAGACATAAACATGCCCGGACATGTCC |
| | | TCGCAATCTAACATGCCCGGACATGTCCTCGCAATCTAACATGCCCGGACATGTCTGCAAG |
| | | CTACAACATGCCCGGACATGTCTACGTACATACTGAAAAGCATACTTTTGCAATGTTATTT |
| | | TTAAAAACAAGGAACTCTTTAACCCAGGGAAGATAATCACTTGGGGAAAGGAAGGTTCGTT |
| | | TCTGAGTTAGCAACAAGTAAATGCAGCACTAGTGGGTGGGATTGAGGTgTGCCCTGGTGCA |
| | | TAAATAGAGACTCAGCTGTGCTGGCACACTCAGAAGCTTGGACCGCATCCTAGCCGCCGAC |
| | | TCACACAAGGCAGGTGGGTGAGGAAATCCAGGTAAGGCTCCTGACAGCAGCTTTAGAAGGG |
| | | TACTTGCTGGAGTGAATTCGGGCCTCTGATTA |
| 415 | SRS019 | GGGGCGGGGtgATGACACAGCAATtcGGGACTTTCCacGCTTGCGTGAGAAGagACCGGAA |
| | | GTgaATGACACAGCAATGGATCCGCTTGCGTGAGAAGctGGGACTTTCCtaGGGGCGGGGt |
| | | tGGGACTTTCCacATGACACAGCAATacCTCGAGGGTACCGGCCCGCCCCCTTTCCTTACG |
| | | CGGATTGGTAGCTGCAGGCTTCCCTATCTGATTGGCCGAACGAACGCAGCGCGTAATTTAA |
| | | AATATTGTATCTGTAACAAAGCTGCACCTCGTGGGCGGAGTTGTGCTCTGCGGCTGCGAAA |
| | | GTCCAGCTTCGGCGACTAGGTGTGAGTAAGCCAGTATCCCAGGAGGAGCAAGTGGCACGTC |
| | | TTCGGGTGAGTGTGCGGCTGTGCTGGAGCCCGGGTTACCAGCTCTT |
| 416 | SRS020 | GGGGCGGGGtgATGACACAGCAATtcGGGACTTTCCacGCTTGCGTGAGAAGagACCGGAA |
| | | GTgaATGACACAGCAATGGATCCGCTTGCGTGAGAAGctGGGACTTTCCtaGGGGCGGGGt |
| | | tGGGACTTTCCacATGACACAGCAATacCTCGAGGGTACCGGGAAAAGTTCAGCTGAGAGA |
| | | TATAAAAGAGCAGTCTTTCCAGCACCTGCAAATCCAGAGCGGCGGGCACTGACGGGCACTT |
| | | GCACCGTGTGGACAGACTCTCCGGTTCTGTGAGTGGTTTTTCTTTTCCCGGGTCGGACCTG |
| | | GAGTTCTTAGGGGGATGGCTG |
| 417 | SRS021 | GGGGCGGGGtgATGACACAGCAATtcGGGACTTTCCacGCTTGCGTGAGAAGagACCGGAA |
| | | GTgaATGACACAGCAATGGATCCGCTTGCGTGAGAAGctGGGACTTTCCtaGGGGCGGGGt |
| | | tGGGACTTTCCacATGACACAGCAATacCTCGAGGGTGACTCATGGGTGACTCATGGGTGA |
| | | CTCATGCTaCgTgTgAcGGTGACTCATGGGTGACTCATGGGTGACTCATGaagTcgcaGat |
| | | tGGTGACTCATGGGTGACTCATGGGTGACTCATGGGTACCGGCCCGCCCCCTTTCCTTACG |
| | | CGGATTGGTAGCTGCAGGCTTCCCTATCTGATTGGCCGAACGAACGCAGCGCGTAATTTAA |
| | | AATATTGTATCTGTAACAAAGCTGCACCTCGTGGGCGGAGTTGTGCTCTGCGGCTGCGAAA |
| | | GTCCAGCTTCGGCGACTAGGTGTGAGTAAGCCAGTATCCCAGGAGGAGCAAGTGGCACGTC |
| | | TTCGGGTGAGTGTGCGGCTGTGCTGGAGCCCGGGTTACCAGCTCTT |
| 418 | SRS022 | GGGGCGGGGtgATGACACAGCAATtcGGGACTTTCCacGCTTGCGTGAGAAGagACCGGAA |
| | | GTgaATGACACAGCAATGGATCCGCTTGCGTGAGAAGctGGGACTTTCCtaGGGGCGGGGt |
| | | tGGGACTTTCCacATGACACAGCAATacCTCGAGGGTGACTCATGATGATGCCACGTCACC |
| | | AATGCCACGTCACCAGGTGACTCATGGGTGACTCATGaCgTgTgAcATGCCACGTCACCAA |
| | | TGCCACGTCACCAGGTGACTCATGGGTGACTCATGGGTACCGGCCCGCCCCCTTTCCTTAC |
| | | GCGGATTGGTAGCTGCAGGCTTCCCTATCTGATTGGCCGAACGAACGCAGCGCGTAATTTA |
| | | AAATATTGTATCTGTAACAAAGCTGCACCTCGTGGGCGGAGTTGTGCTCTGCGGCTGCGAA |
| | | AGTCCAGCTTCGGCGACTAGGTGTGAGTAAGCCAGTATCCCAGGAGGAGCAAGTGGCACGT |
| | | CTTCGGGTGAGTGTGCGGCTGTGCTGGAGCCCGGGTTACCAGCTCTT |
| 419 | SRS023 | GGGGCGGGGtgATGACACAGCAATtcGGGACTTTCCacGCTTGCGTGAGAAGagACCGGAA |
| | | GTgaATGACACAGCAATGGATCCGCTTGCGTGAGAAGctGGGACTTTCCtaGGGGCGGGGt |
| | | tGGGACTTTCCacATGACACAGCAATacCTCGAGGGGAGGAAGTCGTAAAACTTGGGAGGA |
| | | AGTCGTAAAAAATGGGAGGAAGTCGTAAAATGCGGGAGGAAGTCGTAAAAGAAGGGAGGAA |
| | | GTCGTAAAAATCGGGAGGAAGTCGTAAAAGGTACCGGCCCGCCCCCTTTCCTTACGCGGAT |
| | | TGGTAGCTGCAGGCTTCCCTATCTGATTGGCCGAACGAACGCAGCGCGTAATTTAAAATAT |
| | | TGTATCTGTAACAAAGCTGCACCTCGTGGGCGGAGTTGTGCTCTGCGGCTGCGAAAGTCCA |
| | | GCTTCGGCGACTAGGTGTGAGTAAGCCAGTATCCCAGGAGGAGCAAGTGGCACGTCTTCGG |
| | | GTGAGTGTGCGGCTGTGCTGGAGCCCGGGTTACCAGCTCTT |
| 420 | SRS024 | GGGGCGGGGtgATGACACAGCAATtcGGGACTTTCCacGCTTGCGTGAGAAGagACCGGAA |
| | | GTgaATGACACAGCAATGGATCCGCTTGCGTGAGAAGctGGGACTTTCCtaGGGGCGGGGt |
| | | tGGGACTTTCCacATGACACAGCAATacCTCGAGGGTGACTCATGGGTGACTCATGGGTGA |
| | | CTCATGCTaCgTgTgAcGGTGACTCATGGGTGACTCATGGGTGACTCATGaagTcgcaGat |
| | | tGGTGACTCATGGGTGACTCATGGGTGACTCATGGGTACCGGGAAAAGTTCAGCTGAGAGA |
| | | TATAAAAGAGCAGTCTTTCCAGCACCTGCAAATCCAGAGCGGCGGGCACTGACGGGCACTT |
| | | GCACCGTGTGGACAGACTCTCCGGTTCTGTGAGTGGTTTTTCTTTTCCCGGGTCGGACCTG |
| | | GAGTTCTTAGGGGGATGGCTG |
| 421 | SRS025 | GGGGCGGGGtgATGACACAGCAATtcGGGACTTTCCacGCTTGCGTGAGAAGagACCGGAA |
| | | GTgaATGACACAGCAATGGATCCGCTTGCGTGAGAAGctGGGACTTTCCtaGGGGCGGGGt |
| | | tGGGACTTTCCacATGACACAGCAATacCTCGAGGGTGACTCATGATGATGCCACGTCACC |
| | | AATGCCACGTCACCAGGTGACTCATGGGTGACTCATGaCgTgTgAcATGCCACGTCACCAA |
| | | TGCCACGTCACCAGGTGACTCATGGGTGACTCATGGGTACCGGGAAAAGTTCAGCTGAGAG |
| | | ATATAAAAGAGCAGTCTTTCCAGCACCTGCAAATCCAGAGCGGCGGGCACTGACGGGCACT |
| | | TGCACCGTGTGGACAGACTCTCCGGTTCTGTGAGTGGTTTTTCTTTTCCCGGGTCGGACCT |
| | | GGAGTTCTTAGGGGGATGGCTG |
| 422 | SRS026 | ATGACTCAGCAATTAGCGAGTTAGAATGACTCAGCAATTATGCGTCGGACATGACTCAGCA |
| | | ATTACATCTCGATTATGACTCAGCAATTAGGATAGGCATATGACTCAGCAATTACATAGCA |
| | | GCAATGACTCAGCAATTAGCTAGTAAGCTTGGGGCGGGGtgATGACACAGCAATtcGGGAC |
| | | TTTCCacGCTTGCGTGAGAAGagACCGGAAGTgaATGACACAGCAATGGATCCGCTTGCGT |
| | | GAGAAGctGGGACTTTCCtaGGGGCGGGGttGGGACTTTCCacATGACACAGCAATacCTC |
| | | GAGGGTACCGGCCCGCCCCCTTTCCTTACGCGGATTGGTAGCTGCAGGCTTCCCTATCTGA |
| | | TTGGCCGAACGAACGCAGCGCGTAATTTAAAATATTGTATCTGTAACAAAGCTGCACCTCG |
| | | TGGGCGGAGTTGTGCTCTGCGGCTGCGAAAGTCCAGCTTCGGCGACTAGGTGTGAGTAAGC |
| | | CAGTATCCCAGGAGGAGCAAGTGGCACGTCTTCGGGTGAGTGTGCGGCTGTGCTGGAGCCC |
| | | GGGTTACCAGCTCTT |
| 423 | SRS027 | GGGGCGGGGtgATGACACAGCAATtcGGGACTTTCCacGCTTGCGTGAGAAGagACCGGAA |
| | | GTgaATGACACAGCAATGGATCCGCTTGCGTGAGAAGctGGGACTTTCCtaGGGGCGGGGt |
| | | tGGGACTTTCCacATGACACAGCAATacCTCGAGGGTACCCATACTGAAAAGCATACTTTT |
| | | GCAATGTTATTTTTAAAAACAAGGAACTCTTTAACCCAGGGAAGATAATCACTTGGGGAAA |
| | | GGAAGGTTCGTTTCTGAGTTAGCAACAAGTAAATGCAGCACTAGTGGGTGGGATTGAGGTA |
| | | TGCCCTGGTGCATAAATAGAGACTCAGCTGTGCTGGCACACTCAGAAGCTTGGACCGCATC |
| | | CTAGCCGCCGACTCACACAAGGCAGGTGGGTGAGGAAATCCAGGTAAGGCTCCTGACAGCA |
| | | GCTTTAGAAGGGTACTTGCTGGAGTGAATTCGGGCCTCTGATTA |
| 424 | SRS028 | ACATCAAAGGATTTACGGACATCAAAGGATGTACCTACATCAAAGGAATCCTTAACATCAA |
| | | AGGACGCATAGACATCAAAGGAGTTGCGTACATCAAAGGAGCTAGTAAGCTTGGGGCGGGG |
| | | tgATGACACAGCAATtcGGGACTTTCCacGCTTGCGTGAGAAGagACCGGAAGTgaATGAC |
| | | ACAGCAATGGATCCGCTTGCGTGAGAAGctGGGACTTTCCtaGGGGCGGGGttGGGACTTT |
| | | CCacATGACACAGCAATacCTCGAGGGTACCGGCCCGCCCCCTTTCCTTACGCGGATTGGT |
| | | AGCTGCAGGCTTCCCTATCTGATTGGCCGAACGAACGCAGCGCGTAATTTAAAATATTGTA |
| | | TCTGTAACAAAGCTGCACCTCGTGGGCGGAGTTGTGCTCTGCGGCTGCGAAAGTCCAGCTT |
| | | CGGCGACTAGGTGTGAGTAAGCCAGTATCCCAGGAGGAGCAAGTGGCACGTCTTCGGGTGA |
| | | GTGTGCGGCTGTGCTGGAGCCCGGGTTACCAGCTCTT |
| 425 | SRS029 | GGTGACTCATGGGTGACTCATGGGTGACTCATGCTaCgTgTgAcGGTGACTCATGGGTGAC |
| | | TCATGGGTGACTCATGaagTcgcaGattGGTGACTCATGGGTGACTCATGGGTGACTCATG |
| | | ACTAGTAAGCTTGGGGCGGGGtgATGACACAGCAATtcGGGACTTTCCacGCTTGCGTGAG |
| | | AAGagACCGGAAGTgaATGACACAGCAATGGATCCGCTTGCGTGAGAAGctGGGACTTTCC |
| | | taGGGGCGGGGttGGGACTTTCCacATGACACAGCAATacCTCGAGGGTACCGGCCCGCCC |
| | | CCTTTCCTTACGCGGATTGGTAGCTGCAGGCTTCCCTATCTGATTGGCCGAACGAACGCAG |
| | | CGCGTAATTTAAAATATTGTATCTGTAACAAAGCTGCACCTCGTGGGCGGAGTTGTGCTCT |
| | | GCGGCTGCGAAAGTCCAGCTTCGGCGACTAGGTGTGAGTAAGCCAGTATCCCAGGAGGAGC |
| | | AAGTGGCACGTCTTCGGGTGAGTGTGCGGCTGTGCTGGAGCCCGGGTTACCAGCTCTT |
| 426 | SRS030 | GGTGACTCATGATGATGCCACGTCACCAATGCCACGTCACCAGGTGACTCATGGGTGACTC |
| | | ATGaCgTgTgAcATGCCACGTCACCAATGCCACGTCACCAGGTGACTCATGGGTGACTCAT |
| | | GACTAGTAAGCTTGGGGCGGGGtgATGACACAGCAATtcGGGACTTTCCacGCTTGCGTGA |
| | | GAAGagACCGGAAGTgaATGACACAGCAATGGATCCGCTTGCGTGAGAAGctGGGACTTTC |
| | | CtaGGGGCGGGGttGGGACTTTCCacATGACACAGCAATacCTCGAGGGTACCGGCCCGCC |
| | | CCCTTTCCTTACGCGGATTGGTAGCTGCAGGCTTCCCTATCTGATTGGCCGAACGAACGCA |
| | | GCGCGTAATTTAAAATATTGTATCTGTAACAAAGCTGCACCTCGTGGGCGGAGTTGTGCTC |
| | | TGCGGCTGCGAAAGTCCAGCTTCGGCGACTAGGTGTGAGTAAGCCAGTATCCCAGGAGGAG |
| | | CAAGTGGCACGTCTTCGGGTGAGTGTGCGGCTGTGCTGGAGCCCGGGTTACCAGCTCTT |
| 427 | SRS031 | CACTTCCGGTTTACTTCCACTTCCGGTTTACTAGCACTTCCGGTTTACGCTCACTTCCGGT |
| | | TTACGATCACTTCCGGTTTACAGACACTTCCGGTTTACGCTAGTAAGCTTGGGGCGGGGtg |
| | | ATGACACAGCAATtcGGGACTTTCCacGCTTGCGTGAGAAGagACCGGAAGTgaATGACAC |
| | | AGCAATGGATCCGCTTGCGTGAGAAGctGGGACTTTCCtaGGGGCGGGGttGGGACTTTCC |
| | | acATGACACAGCAATacCTCGAGGGTACCGGCCCGCCCCCTTTCCTTACGCGGATTGGTAG |
| | | CTGCAGGCTTCCCTATCTGATTGGCCGAACGAACGCAGCGCGTAATTTAAAATATTGTATC |
| | | TGTAACAAAGCTGCACCTCGTGGGCGGAGTTGTGCTCTGCGGCTGCGAAAGTCCAGCTTCG |
| | | GCGACTAGGTGTGAGTAAGCCAGTATCCCAGGAGGAGCAAGTGGCACGTCTTCGGGTGAGT |
| | | GTGCGGCTGTGCTGGAGCCCGGGTTACCAGCTCTT |
| 428 | SRS032 | ATGACTCAGCAATTAGCGAGTTAGAATGACTCAGCAATTATGCGTCGGACATGACTCAGCA |
| | | ATTACATCTCGATTATGACTCAGCAATTAGGATAGGCATATGACTCAGCAATTACATAGCA |
| | | GCAATGACTCAGCAATTAGCTAGTAAGCTTGGGGCGGGGtgATGACACAGCAATtcGGGAC |
| | | TTTCCacGCTTGCGTGAGAAGagACCGGAAGTgaATGACACAGCAATGGATCCGCTTGCGT |
| | | GAGAAGctGGGACTTTCCtaGGGGCGGGGttGGGACTTTCCacATGACACAGCAATacCTC |
| | | GAGGGTACCGGGAAAAGTTCAGCTGAGAGATATAAAAGAGCAGTCTTTCCAGCACCTGCAA |
| | | ATCCAGAGCGGCGGGCACTGACGGGCACTTGCACCGTGTGGACAGACTCTCCGGTTCTGTG |
| | | AGTGGTTTTTCTTTTCCCGGGTCGGACCTGGAGTTCTTAGGGGGATGGCTG |
| 429 | SRS033 | ACATCAAAGGATTTACGGACATCAAAGGATGTACCTACATCAAAGGAATCCTTAACATCAA |
| | | AGGACGCATAGACATCAAAGGAGTTGCGTACATCAAAGGAGCTAGTAAGCTTGGGGCGGGG |
| | | tgATGACACAGCAATtcGGGACTTTCCacGCTTGCGTGAGAAGagACCGGAAGTgaATGAC |
| | | ACAGCAATGGATCCGCTTGCGTGAGAAGctGGGACTTTCCtaGGGGCGGGGttGGGACTTT |
| | | CCacATGACACAGCAATacCTCGAGGGTACCGGGAAAAGTTCAGCTGAGAGATATAAAAGA |
| | | GCAGTCTTTCCAGCACCTGCAAATCCAGAGCGGCGGGCACTGACGGGCACTTGCACCGTGT |
| | | GGACAGACTCTCCGGTTCTGTGAGTGGTTTTTCTTTTCCCGGGTCGGACCTGGAGTTCTTA |
| | | GGGGGATGGCTG |
| 430 | SRS034 | GGTGACTCATGGGTGACTCATGGGTGACTCATGCTaCgTgTgAcGGTGACTCATGGGTGAC |
| | | TCATGGGTGACTCATGaagTcgcaGattGGTGACTCATGGGTGACTCATGGGTGACTCATG |
| | | ACTAGTAAGCTTGGGGCGGGGtgATGACACAGCAATtcGGGACTTTCCacGCTTGCGTGAG |
| | | AAGagACCGGAAGTgaATGACACAGCAATGGATCCGCTTGCGTGAGAAGctGGGACTTTCC |
| | | taGGGGCGGGGttGGGACTTTCCacATGACACAGCAATacCTCGAGGGTACCGGGAAAAGT |
| | | TCAGCTGAGAGATATAAAAGAGCAGTCTTTCCAGCACCTGCAAATCCAGAGCGGCGGGCAC |
| | | TGACGGGCACTTGCACCGTGTGGACAGACTCTCCGGTTCTGTGAGTGGTTTTTCTTTTCCC |
| | | GGGTCGGACCTGGAGTTCTTAGGGGGATGGCTG |
| 431 | SRS035 | GGTGACTCATGATGATGCCACGTCACCAATGCCACGTCACCAGGTGACTCATGGGTGACTC |
| | | ATGaCgTgTgAcATGCCACGTCACCAATGCCACGTCACCAGGTGACTCATGGGTGACTCAT |
| | | GACTAGTAAGCTTGGGGCGGGGtgATGACACAGCAATtcGGGACTTTCCacGCTTGCGTGA |
| | | GAAGagACCGGAAGTgaATGACACAGCAATGGATCCGCTTGCGTGAGAAGctGGGACTTTC |
| | | CtaGGGGCGGGGttGGGACTTTCCacATGACACAGCAATacCTCGAGGGTACCGGGAAAAG |
| | | TTCAGCTGAGAGATATAAAAGAGCAGTCTTTCCAGCACCTGCAAATCCAGAGCGGCGGGCA |
| | | CTGACGGGCACTTGCACCGTGTGGACAGACTCTCCGGTTCTGTGAGTGGTTTTTCTTTTCC |
| | | CGGGTCGGACCTGGAGTTCTTAGGGGGATGGCTG |
| 432 | SRS036 | GTAAACCGGAAGTGTCTGTAAACCGGAAGTGATCGTAAACCGGAAGTGAGCGTAAACCGGA |
| | | AGTGCTAGTAAACCGGAAGTGGAAGTAAACCGGAAGTGACTAGTAAGCTTGGGGCGGGGtg |
| | | ATGACACAGCAATtcGGGACTTTCCacGCTTGCGTGAGAAGagACCGGAAGTgaATGACAC |
| | | AGCAATGGATCCGCTTGCGTGAGAAGctGGGACTTTCCtaGGGGCGGGGttGGGACTTTCC |
| | | acATGACACAGCAATacCTCGAGGGTACCGGGAAAAGTTCAGCTGAGAGATATAAAAGAGC |
| | | AGTCTTTCCAGCACCTGCAAATCCAGAGCGGCGGGCACTGACGGGCACTTGCACCGTGTGG |
| | | ACAGACTCTCCGGTTCTGTGAGTGGTTTTTCTTTTCCCGGGTCGGACCTGGAGTTCTTAGG |
| | | GGGATGGCTG |
| 433 | SRS037 | GGGGCGGGGtgATGACACAGCAATtcGGGACTTTCCacGCTTGCGTGAGAAGagACCGGAA |
| | | GTgaATGACACAGCAATGGATCCGCGTCCGCCCGAGTCCCCGCCTCGCCGCCAACGCCAAt |
| | | gcTcatGCGTCCGCCCGAGTCCCCGCCTCGCCGCCAACGCCAtcatgcctGCGTCCGCCCG |
| | | AGTCCCCGCCTCGCCGCCAACGCCAGGATCCGCTTGCGTGAGAAGctGGGACTTTCCtaGG |
| | | GGCGGGGttGGGACTTTCCacATGACACAGCAATacCTCGAGGGTGACTCATGATGATGCC |
| | | ACGTCACCAATGCCACGTCACCAGGTGACTCATGGGTGACTCATGaCgTgTgAcATGCCAC |
| | | GTCACCAATGCCACGTCACCAGGTGACTCATGGGTGACTCATGGGTACCGGCCCGCCCCCT |
| | | TTCCTTACGCGGATTGGTAGCTGCAGGCTTCCCTATCTGATTGGCCGAACGAACGCAGCGC |
| | | GTAATTTAAAATATTGTATCTGTAACAAAGCTGCACCTCGTGGGCGGAGTTGTGCTCTGCG |
| | | GCTGCGAAAGTCCAGCTTCGGCGACTAGGTGTGAGTAAGCCAGTATCCCAGGAGGAGCAAG |
| | | TGGCACGTCTTCGGGTGAGTGTGCGGCTGTGCTGGAGCCCGGGTTACCAGCTCTT |
| 434 | SRS038 | ATGACTCAGCAATTAGCGAGTTAGAATGACTCAGCAATTATGCGTCGGACATGACTCAGCA |
| | | ATTACATCTCGATTATGACTCAGCAATTAGGATAGGCATATGACTCAGCAATTACATAGCA |
| | | GCAATGACTCAGCAATTAGCTAGTAAGCTTGGGGCGGGGtgATGACACAGCAATtcGGGAC |
| | | TTTCCacGCTTGCGTGAGAAGagACCGGAAGTgaATGACACAGCAATGGATCCGCTTGCGT |
| | | GAGAAGctGGGACTTTCCtaGGGGCGGGGttGGGACTTTCCacATGACACAGCAATacCTC |
| | | GAGGGTGACTCATGATGATGCCACGTCACCAATGCCACGTCACCAGGTGACTCATGGGTGA |
| | | CTCATGaCgTgTgAcATGCCACGTCACCAATGCCACGTCACCAGGTGACTCATGGGTGACT |
| | | CATGGGTACCGGCCCGCCCCCTTTCCTTACGCGGATTGGTAGCTGCAGGCTTCCCTATCTG |
| | | ATTGGCCGAACGAACGCAGCGCGTAATTTAAAATATTGTATCTGTAACAAAGCTGCACCTC |
| | | GTGGGCGGAGTTGTGCTCTGCGGCTGCGAAAGTCCAGCTTCGGCGACTAGGTGTGAGTAAG |
| | | CCAGTATCCCAGGAGGAGCAAGTGGCACGTCTTCGGGTGAGTGTGCGGCTGTGCTGGAGCC |
| | | CGGGTTACCAGCTCTT |
| 435 | SRS039 | ACATCAAAGGATTTACGGACATCAAAGGATGTACCTACATCAAAGGAATCCTTAACATCAA |
| | | AGGACGCATAGACATCAAAGGAGTTGCGTACATCAAAGGAGCTAGTAAGCTTGGGGCGGGG |
| | | tgATGACACAGCAATtcGGGACTTTCCacGCTTGCGTGAGAAGagACCGGAAGTgaATGAC |
| | | ACAGCAATGGATCCGCTTGCGTGAGAAGctGGGACTTTCCtaGGGGCGGGGttGGGACTTT |
| | | CCacATGACACAGCAATacCTCGAGGGTGACTCATGATGATGCCACGTCACCAATGCCACG |
| | | TCACCAGGTGACTCATGGGTGACTCATGaCgTqTqAcATGCCACGTCACCAATGCCACGTC |
| | | ACCAGGTGACTCATGGGTGACTCATGGGTACCGGCCCGCCCCCTTTCCTTACGCGGATTGG |
| | | TAGCTGCAGGCTTCCCTATCTGATTGGCCGAACGAACGCAGCGCGTAATTTAAAATATTGT |
| | | ATCTGTAACAAAGCTGCACCTCGTGGGCGGAGTTGTGCTCTGCGGCTGCGAAAGTCCAGCT |
| | | TCGGCGACTAGGTGTGAGTAAGCCAGTATCCCAGGAGGAGCAAGTGGCACGTCTTCGGGTG |
| | | AGTGTGCGGCTGTGCTGGAGCCCGGGTTACCAGCTCTT |
| 436 | SRS040 | CACTTCCGGTTTACTTCCACTTCCGGTTTACTAGCACTTCCGGTTTACGCTCACTTCCGGT |
| | | TTACGATCACTTCCGGTTTACAGACACTTCCGGTTTACGCTAGTAAGCTTGGGGCGGGGtg |
| | | ATGACACAGCAATtcGGGACTTTCCacGCTTGCGTGAGAAGagACCGGAAGTgaATGACAC |
| | | AGCAATGGATCCGCTTGCGTGAGAAGctGGGACTTTCCtaGGGGCGGGGttGGGACTTTCC |
| | | acATGACACAGCAATacCTCGAGGGTGACTCATGATGATGCCACGTCACCAATGCCACGTC |
| | | ACCAGGTGACTCATGGGTGACTCATGaCgTgTgAcATGCCACGTCACCAATGCCACGTCAC |
| | | CAGGTGACTCATGGGTGACTCATGGGTACCGGCCCGCCCCCTTTCCTTACGCGGATTGGTA |
| | | GCTGCAGGCTTCCCTATCTGATTGGCCGAACGAACGCAGCGCGTAATTTAAAATATTGTAT |
| | | CTGTAACAAAGCTGCACCTCGTGGGCGGAGTTGTGCTCTGCGGCTGCGAAAGTCCAGCTTC |
| | | GGCGACTAGGTGTGAGTAAGCCAGTATCCCAGGAGGAGCAAGTGGCACGTCTTCGGGTGAG |
| | | TGTGCGGCTGTGCTGGAGCCCGGGTTACCAGCTCTT |
| 437 | SRS041 | GGGGCGGGGtgATGACACAGCAATtcGGGACTTTCCacGCTTGCGTGAGAAGagACCGGAA |
| | | GTgaATGACACAGCAATGGATCCCAACATGGCGGCGCCCAACATGGCGGCTACCAACATGG |
| | | CGGCCTCCAACATGGCGGCAGGCAACATGGCGGCTGCCAACATGGCGGCGGATCCGCTTGC |
| | | GTGAGAAGctGGGACTTTCCtaGGGGCGGGGttGGGACTTTCCacATGACACAGCAATacC |
| | | TCGAGGGTGACTCATGATGATGCCACGTCACCAATGCCACGTCACCAGGTGACTCATGGGT |
| | | GACTCATGaCgTgTgAcATGCCACGTCACCAATGCCACGTCACCAGGTGACTCATGGGTGA |
| | | CTCATGGGTACCGGCCCGCCCCCTTTCCTTACGCGGATTGGTAGCTGCAGGCTTCCCTATC |
| | | TGATTGGCCGAACGAACGCAGCGCGTAATTTAAAATATTGTATCTGTAACAAAGCTGCACC |
| | | TCGTGGGCGGAGTTGTGCTCTGCGGCTGCGAAAGTCCAGCTTCGGCGACTAGGTGTGAGTA |
| | | AGCCAGTATCCCAGGAGGAGCAAGTGGCACGTCTTCGGGTGAGTGTGCGGCTGTGCTGGAG |
| | | CCCGGGTTACCAGCTCTT |
| 438 | SRS042 | GGGGCGGGGtgATGACACAGCAATtcGGGACTTTCCacGCTTGCGTGAGAAGagACCGGAA |
| | | GTgaATGACACAGCAATGGATCCTCCTTTGATGTACGCAACTCCTTTGATGTCTATGCGTC |
| | | CTTTGATGTTAAGGATTCCTTTGATGTAGGTACATCCTTTGATGTCCGTAAATCCTTTGAT |
| | | GTGGATCCGCTTGCGTGAGAAGctGGGACTTTCCtaGGGGCGGGGttGGGACTTTCCacAT |
| | | GACACAGCAATacCTCGAGGGTGACTCATGATGATGCCACGTCACCAATGCCACGTCACCA |
| | | GGTGACTCATGGGTGACTCATGaCgTgTgAcATGCCACGTCACCAATGCCACGTCACCAGG |
| | | TGACTCATGGGTGACTCATGGGTACCGGCCCGCCCCCTTTCCTTACGCGGATTGGTAGCTG |
| | | CAGGCTTCCCTATCTGATTGGCCGAACGAACGCAGCGCGTAATTTAAAATATTGTATCTGT |
| | | AACAAAGCTGCACCTCGTGGGCGGAGTTGTGCTCTGCGGCTGCGAAAGTCCAGCTTCGGCG |
| | | ACTAGGTGTGAGTAAGCCAGTATCCCAGGAGGAGCAAGTGGCACGTCTTCGGGTGAGTGTG |
| | | CGGCTGTGCTGGAGCCCGGGTTACCAGCTCTT |
| 439 | SRS043 | TAATTGCTGAGTCATTGCTGCTATGTAATTGCTGAGTCATATGCCTATCCTAATTGCTGAG |
| | | TCATAATCGAGATGTAATTGCTGAGTCATGTCCGACGCATAATTGCTGAGTCATTCTAACT |
| | | CGCTAATTGCTGAGTCATGTCGACGCTAGCGGTGACTCATGATGATGCCACGTCACCAATG |
| | | CCACGTCACCAGGTGACTCATGGGTGACTCATGaCgTgTgAcATGCCACGTCACCAATGCC |
| | | ACGTCACCAGGTGACTCATGGGTGACTCATGACTAGTAAGCTTGGGGCGGGGtgATGACAC |
| | | AGCAATtcGGGACTTTCCacGCTTGCGTGAGAAGagACCGGAAGTgaATGACACAGCAATG |
| | | GATCCGCTTGCGTGAGAAGctGGGACTTTCCtaGGGGCGGGGttGGGACTTTCCacATGAC |
| | | ACAGCAATacCTCGAGGGTACCGGCCCGCCCCCTTTCCTTACGCGGATTGGTAGCTGCAGG |
| | | CTTCCCTATCTGATTGGCCGAACGAACGCAGCGCGTAATTTAAAATATTGTATCTGTAACA |
| | | AAGCTGCACCTCGTGGGCGGAGTTGTGCTCTGCGGCTGCGAAAGTCCAGCTTCGGCGACTA |
| | | GGTGTGAGTAAGCCAGTATCCCAGGAGGAGCAAGTGGCACGTCTTCGGGTGAGTGTGCGGC |
| | | TGTGCTGGAGCCCGGGTTACCAGCTCTT |
| 440 | SRS044 | TAATTGCTGAGTCATTGCTGCTATGTAATTGCTGAGTCATATGCCTATCCTCCTTTGATGT |
| | | ACGCAACTCCTTTGATGTCTATGCGTAATTGCTGAGTCATAATCGAGATGTAATTGCTGAG |
| | | TCATGTCCGACGCATCCTTTGATGTTAAGGATTCCTTTGATGTAGGTACATAATTGCTGAG |
| | | TCATTCTAACTCGCTAATTGCTGAGTCATcatCtcgAcCTCCTTTGATGTCCGTAAATCCT |
| | | TTGATGTGTCGACGCTAGCGGTGACTCATGATGATGCCACGTCACCAATGCCACGTCACCA |
| | | GGTGACTCATGGGTGACTCATGaCgTgTgAcATGCCACGTCACCAATGCCACGTCACCAGG |
| | | TGACTCATGGGTGACTCATGACTAGTAAGCTTGGGGCGGGGtgATGACACAGCAATtcGGG |
| | | ACTTTCCacGCTTGCGTGAGAAGagACCGGAAGTgaATGACACAGCAATGGATCCGCTTGC |
| | | GTGAGAAGctGGGACTTTCCtaGGGGCGGGGttGGGACTTTCCacATGACACAGCAATacC |
| | | TCGAGGGTACCGGCCCGCCCCCTTTCCTTACGCGGATTGGTAGCTGCAGGCTTCCCTATCT |
| | | GATTGGCCGAACGAACGCAGCGCGTAATTTAAAATATTGTATCTGTAACAAAGCTGCACCT |
| | | CGTGGGCGGAGTTGTGCTCTGCGGCTGCGAAAGTCCAGCTTCGGCGACTAGGTGTGAGTAA |
| | | GCCAGTATCCCAGGAGGAGCAAGTGGCACGTCTTCGGGTGAGTGTGCGGCTGTGCTGGAGC |
| | | CCGGGTTACCAGCTCTT |
| 441 | SRS045 | TAATTGCTGAGTCATTGCTGCTATGTAATTGCTGAGTCATATGCCTATCCTAATTGCTGAG |
| | | TCATAATCGAGATGTAATTGCTGAGTCATGTCCGACGCATAATTGCTGAGTCATTCTAACT |
| | | CGCTAATTGCTGAGTCATGTCGACACTAGTAAGCTTGGGGCGGGGtgATGACACAGCAATt |
| | | CGGGACTTTCCacGCTTGCGTGAGAAGagACCGGAAGTgaATGACACAGCAATGGATCCGC |
| | | TTGCGTGAGAAGctGGGACTTTCCtaGGGGCGGGGttGGGACTTTCCacATGACACAGCAA |
| | | TacCTCGAGGGTACCGGCCCGCCCCCTTTCCTTACGCGGATTGGTAGCTGCAGGCTTCCCT |
| | | ATCTGATTGGCCGAACGAACGCAGCGCGTAATTTAAAATATTGTATCTGTAACAAAGCTGC |
| | | ACCTCGTGGGCGGAGTTGTGCTCTGCGGCTGCGAAAGTCCAGCTTCGGCGACTAGGTGTGA |
| | | GTAAGCCAGTATCCCAGGAGGAGCAAGTGGCACGTCTTCGGGTGAGTGTGCGGCTGTGCTG |
| | | GAGCCCGGGTTACCAGCTCTT |
| 442 | SRS046 | GGTGACTCATGATGATGCCACGTCACCAATGCCACGTCACCAGGTGACTCATGGGTGACTC |
| | | ATGaCgTgTgAcATGCCACGTCACCAATGCCACGTCACCAGGTGACTCATGGGTGACTCAT |
| | | GACTAGTGAATTCTAATTGCTGAGTCATTGCTGCTATGTAATTGCTGAGTCATATGCCTAT |
| | | CCTCCTTTGATGTACGCAACTCCTTTGATGTCTATGCGTAATTGCTGAGTCATAATCGAGA |
| | | TGTAATTGCTGAGTCATGTCCGACGCATCCTTTGATGTTAAGGATTCCTTTGATGTAGGTA |
| | | CATAATTGCTGAGTCATTCTAACTCGCTAATTGCTGAGTCATcatCtcgAcCTCCTTTGAT |
| | | GTCCGTAAATCCTTTGATGTGTCGACACTAGTAAGCTTGGGGCGGGGtgATGACACAGCAA |
| | | TtcGGGACTTTCCacGCTTGCGTGAGAAGagACCGGAAGTgaATGACACAGCAATGGATCC |
| | | GCTTGCGTGAGAAGctGGGACTTTCCtaGGGGCGGGGttGGGACTTTCCacATGACACAGC |
| | | AATacCTCGAGGGTACCGGCCCGCCCCCTTTCCTTACGCGGATTGGTAGCTGCAGGCTTCC |
| | | CTATCTGATTGGCCGAACGAACGCAGCGCGTAATTTAAAATATTGTATCTGTAACAAAGCT |
| | | GCACCTCGTGGGCGGAGTTGTGCTCTGCGGCTGCGAAAGTCCAGCTTCGGCGACTAGGTGT |
| | | GAGTAAGCCAGTATCCCAGGAGGAGCAAGTGGCACGTCTTCGGGTGAGTGTGCGGCTGTGC |
| | | TGGAGCCCGGGTTACCAGCTCTT |
| 443 | SRS047 | TCCTTTGATGTACGCAACTCCTTTGATGTCTATGCGTCCTTTGATGTTAAGGATTCCTTTG |
| | | ATGTAGGTACATCCTTTGATGTCCGTAAATCCTTTGATGTGTCGACGCTAGCGGTGACTCA |
| | | TGATGATGCCACGTCACCAATGCCACGTCACCAGGTGACTCATGGGTGACTCATGaCgTgT |
| | | gAcATGCCACGTCACCAATGCCACGTCACCAGGTGACTCATGGGTGACTCATGACTAGTAA |
| | | GCTTGGGGCGGGGtgATGACACAGCAATtcGGGACTTTCCacGCTTGCGTGAGAAGagACC |
| | | GGAAGTgaATGACACAGCAATGGATCCGCTTGCGTGAGAAGctGGGACTTTCCtaGGGGCG |
| | | GGGttGGGACTTTCCacATGACACAGCAATacCTCGAGGGTACCGGCCCGCCCCCTTTCCT |
| | | TACGCGGATTGGTAGCTGCAGGCTTCCCTATCTGATTGGCCGAACGAACGCAGCGCGTAAT |
| | | TTAAAATATTGTATCTGTAACAAAGCTGCACCTCGTGGGCGGAGTTGTGCTCTGCGGCTGC |
| | | GAAAGTCCAGCTTCGGCGACTAGGTGTGAGTAAGCCAGTATCCCAGGAGGAGCAAGTGGCA |
| | | CGTCTTCGGGTGAGTGTGCGGCTGTGCTGGAGCCCGGGTTACCAGCTCTT |
| 444 | SRS048 | GGTGACTCATGATGATGCCACGTCACCAATGCCACGTCACCAGGTGACTCATGGGTGACTC |
| | | ATGaCgTgTgAcATGCCACGTCACCAATGCCACGTCACCAGGTGACTCATGGGTGACTCAT |
| | | GACTAGTGAATTCGACTCCTTTGATGTACGCAACTCCTTTGATGTCTATGCGTCCTTTGAT |
| | | GTTAAGGATTCCTTTGATGTAGGTACATCCTTTGATGTCCGTAAATCCTTTGATGTGTCGA |
| | | CACTAGTAAGCTTGGGGCGGGGtgATGACACAGCAATtcGGGACTTTCCacGCTTGCGTGA |
| | | GAAGagACCGGAAGTgaATGACACAGCAATGGATCCGCTTGCGTGAGAAGctGGGACTTTC |
| | | CtaGGGGCGGGGttGGGACTTTCCacATGACACAGCAATacCTCGAGGGTACCGGCCCGCC |
| | | CCCTTTCCTTACGCGGATTGGTAGCTGCAGGCTTCCCTATCTGATTGGCCGAACGAACGCA |
| | | GCGCGTAATTTAAAATATTGTATCTGTAACAAAGCTGCACCTCGTGGGCGGAGTTGTGCTC |
| | | TGCGGCTGCGAAAGTCCAGCTTCGGCGACTAGGTGTGAGTAAGCCAGTATCCCAGGAGGAG |
| | | CAAGTGGCACGTCTTCGGGTGAGTGTGCGGCTGTGCTGGAGCCCGGGTTACCAGCTCTT |
| 445 | SRS049 | ATGACTCAGCAATTAGCGAGTTAGAATGACTCAGCAATTATGCGTCGGACATGACTCAGCA |
| | | ATTACATCTCGATTATGACTCAGCAATTAGGATAGGCATATGACTCAGCAATTACATAGCA |
| | | GCAATGACTCAGCAATTAGCTAGTAAGCTTGGGGCGGGGtgATGACACAGCAATtcGGGAC |
| | | TTTCCacGCTTGCGTGAGAAGagACCGGAAGTgaATGACACAGCAATGGATCCGCTTGCGT |
| | | GAGAAGctGGGACTTTCCtaGGGGCGGGGttGGGACTTTCCacATGACACAGCAATacCTC |
| | | GAGGGTACCGGGAAAAGTTCAGCTGAGAGATATAAAAGAGCAGTCTTTCCAGCACCTGCGT |
| | | ATCCCAGGAGGAGCAAGTGGCACGTCTTCGGGTGAGTGTGCGGCTGTGCTGGAGCCCGGGT |
| | | TACCAGCTCTTA |
| 446 | SRS050 | ATGACTCAGCAATTAGCGAGTTAGAATGACTCAGCAATTATGCGTCGGACATGACTCAGCA |
| | | ATTACATCTCGATTATGACTCAGCAATTAGGATAGGCATATGACTCAGCAATTACATAGCA |
| | | GCAATGACTCAGCAATTAGCTAGTAAGCTTGGGGCGGGGtgATGACACAGCAATtcGGGAC |
| | | TTTCCacGCTTGCGTGAGAAGagACCGGAAGTgaATGACACAGCAATGGATCCGCTTGCGT |
| | | GAGAAGctGGGACTTTCCtaGGGGCGGGGttGGGACTTTCCacATGACACAGCAATacCTC |
| | | GAGGGTACCtgcgctcccgacatgccccgcggcgcgccattaaccgccagatttgagtcgc |
| | | gggacccgttggcagaggtgg |
| 447 | SRS051 | GGTGACTCATGATGATGCCACGTCACCAATGCCACGTCACCAGGTGACTCATGGGTGACTC |
| | | ATGaCgTgTgAcATGCCACGTCACCAATGCCACGTCACCAGGTGACTCATGGGTGACTCAT |
| | | GACTAGTAAGCTTGGGGCGGGGtgATGACACAGCAATtcGGGACTTTCCacGCTTGCGTGA |
| | | GAAGagACCGGAAGTgaATGACACAGCAATGGATCCGCTTGCGTGAGAAGctGGGACTTTC |
| | | CtaGGGGCGGGGttGGGACTTTCCacATGACACAGCAATacCTCGAGGGTACCGGGAAAAG |
| | | TTCAGCTGAGAGATATAAAAGAGCAGTCTTTCCAGCACCTGCGTATCCCAGGAGGAGCAAG |
| | | TGGCACGTCTTCGGGTGAGTGTGCGGCTGTGCTGGAGCCCGGGTTACCAGCTCTTA |
| 448 | SRS052 | GGTGACTCATGATGATGCCACGTCACCAATGCCACGTCACCAGGTGACTCATGGGTGACTC |
| | | ATGaCgTgTgAcATGCCACGTCACCAATGCCACGTCACCAGGTGACTCATGGGTGACTCAT |
| | | GACTAGTAAGCTTGGGGCGGGGtgATGACACAGCAATtcGGGACTTTCCacGCTTGCGTGA |
| | | GAAGagACCGGAAGTgaATGACACAGCAATGGATCCGCTTGCGTGAGAAGctGGGACTTTC |
| | | CtaGGGGCGGGGttGGGACTTTCCacATGACACAGCAATacCTCGAGGGTACCtgcgctcc |
| | | cgacatgccccgcggcgcgccattaaccgccagatttgagtcgcgggacccgttggcagag |
| | | gtgg |
| 449 | SRS053 | ATGACTCAGCAATTAGCGAGTTAGAATGACTCAGCAATTATGCGTCGGACATGACTCAGCA |
| | | ATTACATCTCGATTATGACTCAGCAATTAGGATAGGCATATGACTCAGCAATTACATAGCA |
| | | GCAATGACTCAGCAATTAGCTAGTAAGCTTGGGGCGGGGtgATGACACAGCAATtcGGGAC |
| | | TTTCCacGCTTGCGTGAGAAGagACCGGAAGTgaATGACACAGCAATGGATCCGGGAGGAA |
| | | GTCGTAAAACTTGGGAGGAAGTCGTAAAAAATGGGAGGAAGTCGTAAAATGCGGGAGGAAG |
| | | TCGTAAAAGAAGGGAGGAAGTCGTAAAAATCGGGAGGAAGTCGTAAAAGGATCCGCTTGCG |
| | | TGAGAAGctGGGACTTTCCtaGGGGCGGGGttGGGACTTTCCacATGACACAGCAATacCT |
| | | CGAGGGTACCGGCCCGCCCCCTTTCCTTACGCGGATTGGTAGCTGCAGGCTTCCCTATCTG |
| | | ATTGGCCGAACGAACGCAGCGCGTAATTTAAAATATTGTATCTGTAACAAAGCTGCACCTC |
| | | GTGGGCGGAGTTGTGCTCTGCGGCTGCGAAAGTCCAGCTTCGGCGACTAGGTGTGAGTAAG |
| | | CCAGTATCCCAGGAGGAGCAAGTGGCACGTCTTCGGGTGAGTGTGCGGCTGTGCTGGAGCC |
| | | CGGGTTACCAGCTCTT |
| 450 | SRS054 | ATGACTCAGCAATTAGCGAGTTAGAATGACTCAGCAATTATGCGTCGGACATGACTCAGCA |
| | | ATTACATCTCGATTATGACTCAGCAATTAGGATAGGCATATGACTCAGCAATTACATAGCA |
| | | GCAATGACTCAGCAATTAGCTAGTAAGCTTGGGGCGGGGtgATGACACAGCAATtcGGGAC |
| | | TTTCCacGCTTGCGTGAGAAGagACCGGAAGTgaATGACACAGCAATGGATCCTCCTTTGA |
| | | TGTACGCAACTCCTTTGATGTCTATGCGTCCTTTGATGTTAAGGATTCCTTTGATGTAGGT |
| | | ACATCCTTTGATGTCCGTAAATCCTTTGATGTGGATCCGCTTGCGTGAGAAGctGGGACTT |
| | | TCCtaGGGGCGGGGttGGGACTTTCCacATGACACAGCAATacCTCGAGGGTACCGGCCCG |
| | | CCCCCTTTCCTTACGCGGATTGGTAGCTGCAGGCTTCCCTATCTGATTGGCCGAACGAACG |
| | | CAGCGCGTAATTTAAAATATTGTATCTGTAACAAAGCTGCACCTCGTGGGCGGAGTTGTGC |
| | | TCTGCGGCTGCGAAAGTCCAGCTTCGGCGACTAGGTGTGAGTAAGCCAGTATCCCAGGAGG |
| | | AGCAAGTGGCACGTCTTCGGGTGAGTGTGCGGCTGTGCTGGAGCCCGGGTTACCAGCTCTT |
| 451 | SRS055 | GGTGACTCATGATGATGCCACGTCACCAATGCCACGTCACCAGGTGACTCATGGGTGACTC |
| | | ATGaCgTgTgAcATGCCACGTCACCAATGCCACGTCACCAGGTGACTCATGGGTGACTCAT |
| | | GACTAGTAAGCTTGGGGCGGGGtgATGACACAGCAATtcGGGACTTTCCacGCTTGCGTGA |
| | | GAAGagACCGGAAGTgaATGACACAGCAATGGATCCTTTTACGACTTCCTCCCGATTTTTA |
| | | CGACTTCCTCCCTTCTTTTACGACTTCCTCCCGCATTTTACGACTTCCTCCCATTTTTTAC |
| | | GACTTCCTCCCAAGTTTTACGACTTCCTCCCGGATCCGCTTGCGTGAGAAGctGGGACTTT |
| | | CCtaGGGGCGGGGttGGGACTTTCCacATGACACAGCAATacCTCGAGGGTACCGGCCCGC |
| | | CCCCTTTCCTTACGCGGATTGGTAGCTGCAGGCTTCCCTATCTGATTGGCCGAACGAACGC |
| | | AGCGCGTAATTTAAAATATTGTATCTGTAACAAAGCTGCACCTCGTGGGCGGAGTTGTGCT |
| | | CTGCGGCTGCGAAAGTCCAGCTTCGGCGACTAGGTGTGAGTAAGCCAGTATCCCAGGAGGA |
| | | GCAAGTGGCACGTCTTCGGGTGAGTGTGCGGCTGTGCTGGAGCCCGGGTTACCAGCTCTT |
| 452 | SRS056 | GGTGACTCATGATGATGCCACGTCACCAATGCCACGTCACCAGGTGACTCATGGGTGACTC |
| | | ATGaCgTgTgAcATGCCACGTCACCAATGCCACGTCACCAGGTGACTCATGGGTGACTCAT |
| | | GACTAGTAAGCTTGGGGCGGGGtgATGACACAGCAATtcGGGACTTTCCacGCTTGCGTGA |
| | | GAAGagACCGGAAGTgaATGACACAGCAATGGATCCTCCTTTGATGTACGCAACTCCTTTG |
| | | ATGTCTATGCGTCCTTTGATGTTAAGGATTCCTTTGATGTAGGTACATCCTTTGATGTCCG |
| | | TAAATCCTTTGATGTGGATCCGCTTGCGTGAGAAGctGGGACTTTCCtaGGGGCGGGGttG |
| | | GGACTTTCCacATGACACAGCAATacCTCGAGGGTACCGGCCCGCCCCCTTTCCTTACGCG |
| | | GATTGGTAGCTGCAGGCTTCCCTATCTGATTGGCCGAACGAACGCAGCGCGTAATTTAAAA |
| | | TATTGTATCTGTAACAAAGCTGCACCTCGTGGGCGGAGTTGTGCTCTGCGGCTGCGAAAGT |
| | | CCAGCTTCGGCGACTAGGTGTGAGTAAGCCAGTATCCCAGGAGGAGCAAGTGGCACGTCTT |
| | | CGGGTGAGTGTGCGGCTGTGCTGGAGCCCGGGTTACCAGCTCTT |
| 453 | SRS057 | ATGACTCAGCAATTAGCGAGTTAGAATGACTCAGCAATTATGCGTCGGACATGACTCAGCA |
| | | ATTACATCTCGATTATGACTCAGCAATTAGGATAGGCATATGACTCAGCAATTACATAGCA |
| | | GCAATGACTCAGCAATTAGCTAGTAAGCTTGGGGCGGGGtqATGACACAGCAATtcGGGAC |
| | | TTTCCacGCTTGCGTGAGAAGagACCGGAAGTgaATGACACAGCAATGGATCCGCTTGCGT |
| | | GAGAAGctGGGACTTTCCtaGGGGCGGGGttGGGACTTTCCacATGACACAGCAATacCTC |
| | | GAGGGTACCTATAAAAGGCCAGCAGCAGCCTGACCACATCTCATCC |
| 454 | SRS058 | GGTGACTCATGATGATGCCACGTCACCAATGCCACGTCACCAGGTGACTCATGGGTGACTC |
| | | ATGaCgTgTgAcATGCCACGTCACCAATGCCACGTCACCAGGTGACTCATGGGTGACTCAT |
| | | GACTAGTAAGCTTGGGGCGGGGtgATGACACAGCAATtcGGGACTTTCCacGCTTGCGTGA |
| | | GAAGagACCGGAAGTgaATGACACAGCAATGGATCCGCTTGCGTGAGAAGctGGGACTTTC |
| | | CtaGGGGCGGGGttGGGACTTTCCacATGACACAGCAATacCTCGAGGGTACCTATAAAAG |
| | | GCCAGCAGCAGCCTGACCACATCTCATCC |
| 455 | SRS059 | ATGACTCAGCAATTAGCGAGTTAGAATGACTCAGCAATTATGCGTCGGACATGACTCAGCA |
| | | ATTACATCTCGATTATGACTCAGCAATTAGGATAGGCATATGACTCAGCAATTACATAGCA |
| | | GCAATGACTCAGCAATTAGCTAGTAAGCTTGGGGCGGGGtgATGACACAGCAATtcGGGAC |
| | | TTTCCacGCTTGCGTGAGAAGagACCGGAAGTgaATGACACAGCAATGGATCCGCTTGCGT |
| | | GAGAAGctGGGACTTTCCtaGGGGCGGGGttGGGACTTTCCacATGACACAGCAATacCTC |
| | | GAGGGTACCACCTCTTAACAATACGTTTCACAAATAGTTAAAAACATGCATACTGAAAAGC |
| | | ATACTTTTGCAATGTTATTTTTAAAAACAAGGAACTCTTTAACCCAGGGAAGATAATCACT |
| | | TGGGGAAAGGAAGGTTCGTTTCTGAGTTAGCAACAAGTAAATGCAGCACTAGTGGGTGGGA |
| | | TTGAGGTgTGCCCTGGTGCATAAATAGAGACTCAGCTGTGCTGGCACACTCAAGAAGCTTG |
| | | GACCGCATCCTAGCCGCCGACTCACACAAGGCAGGTGGGTGAGGAAATCCAGGTAAGGCTC |
| | | CTGACAGCAGCTTTAGAAGGGTACTTGCTGGAGTGAATTCGGGCCTCTGATT |
| 456 | SRS060 | GGTGACTCATGATGATGCCACGTCACCAATGCCACGTCACCAGGTGACTCATGGGTGACTC |
| | | ATGaCgTgTgAcATGCCACGTCACCAATGCCACGTCACCAGGTGACTCATGGGTGACTCAT |
| | | GACTAGTAAGCTTGGGGCGGGGtqATGACACAGCAATtcGGGACTTTCCacGCTTGCGTGA |
| | | GAAGagACCGGAAGTgaATGACACAGCAATGGATCCGCTTGCGTGAGAAGctGGGACTTTC |
| | | CtaGGGGCGGGGttGGGACTTTCCacATGACACAGCAATacCTCGAGGGTACCACCTCTTA |
| | | ACAATACGTTTCACAAATAGTTAAAAACATGCATACTGAAAAGCATACTTTTGCAATGTTA |
| | | TTTTTAAAAACAAGGAACTCTTTAACCCAGGGAAGATAATCACTTGGGGAAAGGAAGGTTC |
| | | GTTTCTGAGTTAGCAACAAGTAAATGCAGCACTAGTGGGTGGGATTGAGGTgTGCCCTGGT |
| | | GCATAAATAGAGACTCAGCTGTGCTGGCACACTCAAGAAGCTTGGACCGCATCCTAGCCGC |
| | | CGACTCACACAAGGCAGGTGGGTGAGGAAATCCAGGTAAGGCTCCTGACAGCAGCTTTAGA |
| | | AGGGTACTTGCTGGAGTGAATTCGGGCCTCTGATT |
| 457 | SRS061 | TAATTGCTGAGTCATTGCTGCTATGTAATTGCTGAGTCATATGCCTATCCTAATTGCTGAG |
| | | TCATAATCGAGATGTAATTGCTGAGTCATGTCCGACGCATAATTGCTGAGTCATTCTAACT |
| | | CGCTAATTGCTGAGTCATGTCGACGCTAGCGGTGACTCATGATGATGCCACGTCACCAATG |
| | | CCACGTCACCAGGTGACTCATGGGTGACTCATGaCgTgTgAcATGCCACGTCACCAATGCC |
| | | ACGTCACCAGGTGACTCATGGGTGACTCATGACTAGTTCCTTTGATGTACGCAACTCCTTT |
| | | GATGTCTATGCGTCCTTTGATGTTAAGGATTCCTTTGATGTAGGTACATCCTTTGATGTCC |
| | | GTAAATCCTTTGATGTCTCGAGGGTACCGGCCCGCCCCCTTTCCTTACGCGGATTGGTAGC |
| | | TGCAGGCTTCCCTATCTGATTGGCCGAACGAACGCAGCGCGTAATTTAAAATATTGTATCT |
| | | GTAACAAAGCTGCACCTCGTGGGCGGAGTTGTGCTCTGCGGCTGCGAAAGTCCAGCTTCGG |
| | | CGACTAGGTGTGAGTAAGCCAGTATCCCAGGAGGAGCAAGTGGCACGTCTTCGGGTGAGTG |
| | | TGCGGCTGTGCTGGAGCCCGGGTTACCAGCTCTT |
| 458 | SRS062 | TAATTGCTGAGTCATTGCTGCTATGTAATTGCTGAGTCATATGCCTATCCTAATTGCTGAG |
| | | TCATAATCGAGATGTAATTGCTGAGTCATGTCCGACGCATAATTGCTGAGTCATTCTAACT |
| | | CGCTAATTGCTGAGTCATGTCGACGCTAGCGGTGACTCATGATGATGCCACGTCACCAATG |
| | | CCACGTCACCAGGTGACTCATGGGTGACTCATGaCgTgTgAcATGCCACGTCACCAATGCC |
| | | ACGTCACCAGGTGACTCATGGGTGACTCATGACTAGTCAACATGGCGGCGCCCAACATGGC |
| | | GGCTACCAACATGGCGGCCTCCAACATGGCGGCAGGCAACATGGCGGCTGCCAACATGGCG |
| | | GCCTCGAGGGTACCGGCCCGCCCCCTTTCCTTACGCGGATTGGTAGCTGCAGGCTTCCCTA |
| | | TCTGATTGGCCGAACGAACGCAGCGCGTAATTTAAAATATTGTATCTGTAACAAAGCTGCA |
| | | CCTCGTGGGCGGAGTTGTGCTCTGCGGCTGCGAAAGTCCAGCTTCGGCGACTAGGTGTGAG |
| | | TAAGCCAGTATCCCAGGAGGAGCAAGTGGCACGTCTTCGGGTGAGTGTGCGGCTGTGCTGG |
| | | AGCCCGGGTTACCAGCTCTT |
| 459 | SRS063 | TCCTTTGATGTACGCAACTCCTTTGATGTCTATGCGTCCTTTGATGTTAAGGATTCCTTTG |
| | | ATGTAGGTACATCCTTTGATGTCCGTAAATCCTTTGATGTGTCGACGCTAGCGGTGACTCA |
| | | TGATGATGCCACGTCACCAATGCCACGTCACCAGGTGACTCATGGGTGACTCATGaCgTgT |
| | | gAcATGCCACGTCACCAATGCCACGTCACCAGGTGACTCATGGGTGACTCATGACTAGTTA |
| | | ATTGCTGAGTCATTGCTGCTATGTAATTGCTGAGTCATATGCCTATCCTAATTGCTGAGTC |
| | | ATAATCGAGATGTAATTGCTGAGTCATGTCCGACGCATAATTGCTGAGTCATTCTAACTCG |
| | | CTAATTGCTGAGTCATCTCGAGGGTACCGGCCCGCCCCCTTTCCTTACGCGGATTGGTAGC |
| | | TGCAGGCTTCCCTATCTGATTGGCCGAACGAACGCAGCGCGTAATTTAAAATATTGTATCT |
| | | GTAACAAAGCTGCACCTCGTGGGCGGAGTTGTGCTCTGCGGCTGCGAAAGTCCAGCTTCGG |
| | | CGACTAGGTGTGAGTAAGCCAGTATCCCAGGAGGAGCAAGTGGCACGTCTTCGGGTGAGTG |
| | | TGCGGCTGTGCTGGAGCCCGGGTTACCAGCTCTT |
| 460 | SRS064 | AcgcGtcccgacatgccccgcggcgcgccattaaccgccagatttgagtcgcgggacccgt |
| | | tggcagaggtgg |
| 461 | SRS065 | GGGGCGGGGtgATGACACAGCAATtcGGGACTTTCCacGCTTGCGTGAGAAGagACCGGAA |
| | | GTgaATGACACAGCAATGGATCCGCTTGCGTGAGAAGctGGGACTTTCCtaGGGGCGGGGt |
| | | tGGGACTTTCCacATGACACAGCAATacCTCGAGGGTGACTCATGATGATGCCACGTCACC |
| | | AATGCCACGTCACCAGGTGACTCATGGGTGACTCATGaCqTgTgAcATGCCACGTCACCAA |
| | | TGCCACGTCACCAGGTGACTCATGGGTGACTCATGGGTACCACCTCTTAACAATACGTTTC |
| | | ACAAATAGTTAAAAACATGCATACTGAAAAGCATACTTTTGCAATGTTATTTTTAAAAACA |
| | | AGGAACTCTTTAACCCAGGGAAGATAATCACTTGGGGAAAGGAAGGTTCGTTTCTGAGTTA |
| | | GCAACAAGTAAATGCAGCACTAGTGGGTGGGATTGAGGTgTGCCCTGGTGCATAAATAGAG |
| | | ACTCAGCTGTGCTGGCACACTCAAAAATCCAGAGCGGCGGGCACTGACGGGCACTTGCACC |
| | | GTGTGGACAGACTCTCCGGTTCTGTGAGTGGTTTTTCTTTTCCCGGGTCGGACCTGGAGTT |
| | | CTTAGGGGGATGGCTGAAgaattcA |
| 462 | SRS066 | GGGGCGGGGtgATGACACAGCAATtcGGGACTTTCCacGCTTGCGTGAGAAGagACCGGAA |
| | | GTgaATGACACAGCAATGGATCCGCTTGCGTGAGAAGctGGGACTTTCCtaGGGGCGGGGt |
| | | tGGGACTTTCCacATGACACAGCAATacCTCGAGGGTGACTCATGATGATGCCACGTCACC |
| | | AATGCCACGTCACCAGGTGACTCATGGGTGACTCATGaCgTgTgAcATGCCACGTCACCAA |
| | | TGCCACGTCACCAGGTGACTCATGGGTGACTCATGGGTACCACCTCTTAACAATACGTTTC |
| | | ACAAATAGTTAAAAACATGCATACTGAAAAGCATACTTTTGCAATGTTATTTTTAAAAACA |
| | | AGGAACTCTTTAACCCAGGGAAGATAATCACTTGGGGAAAGGAAGGTTCGTTTCTGAGTTA |
| | | GCAACAAGTAAATGCAGCACTAGTGGGTGGGATTGAGGTgTGCCCTGGTGCATAAATAGAG |
| | | ACTCAGCTGTGCTGGCACACTCAAGAAGCTTGGACCGCATCCTAGCCGCCGACTCACACAA |
| | | GGCAGGTGGGTGAGGAAATCCAGGTAAGGCTCCTGACAGCAGCTTTAGAAGGGTACTTGCT |
| | | GGAGTGAATTCGGGCCTCTGATTA |
| 463 | SRS067 | GGGGCGGGGtgATGACACAGCAATtcGGGACTTTCCacGCTTGCGTGAGAAGagACCGGAA |
| | | GTgaATGACACAGCAATGGATCCGCTTGCGTGAGAAGctGGGACTTTCCtaGGGGCGGGGt |
| | | tGGGACTTTCCacATGACACAGCAATacCTCGAGGGTGACTCATGATGATGCCACGTCACC |
| | | AATGCCACGTCACCAGGTGACTCATGGGTGACTCATGaCgTgTgAcATGCCACGTCACCAA |
| | | TGCCACGTCACCAGGTGACTCATGGGTGACTCATGGGTACCACCTCTTAACAATACGTTTC |
| | | ACAAATAGTTAAAAACATGCATACTGAAAAGCATACTTTTGCAATGTTATTTTTAAAAACA |
| | | AGGAACTCTTTAACCCAGGGAAGATAATCACTTGGGGAAAGGAAGGTTCGTTTCTGAGTTA |
| | | GCAACAAGTAAATGCAGCACTAGTGGGTGGGATTGAGGTgTGCCCTGGTGCATAAATAGAG |
| | | ACTCAGCTGTGCTGGCACACTCAAGTATCCCAGGAGGAGCAAGTGGCACGTCTTCGGGTGA |
| | | GTGTGCGGCTGTGCTGGAGCCCGGGTTACCAGCTCTTAA |
| 464 | SRS068 | GGGGCGGGGtgATGACACAGCAATtcGGGACTTTCCacGCTTGCGTGAGAAGagACCGGAA |
| | | GTgaATGACACAGCAATGGATCCGCTTGCGTGAGAAGctGGGACTTTCCtaGGGGCGGGGt |
| | | tGGGACTTTCCacATGACACAGCAATacCTCGAGGGTGACTCATGATGATGCCACGTCACC |
| | | AATGCCACGTCACCAGGTGACTCATGGGTGACTCATGaCgTgTgAcATGCCACGTCACCAA |
| | | TGCCACGTCACCAGGTGACTCATGGGTGACTCATGGGTACCACCTCTTAACAATACGTTTC |
| | | ACAAATAGTTAAAAACATGCATACTGAAAAGCATACTTTTGCAATGTTATTTTTAAAAACA |
| | | AGGAACTCTTTAACCCAGGGAAGATAATCACTTGGGGAAAGGAAGGTTCGTTTCTGAGTTA |
| | | GCAACAAGTAAATGCAGCACTAGTGGGTGGGATTGAGGTgTGCCCTGGTGCATAAATAGAG |
| | | ACTCAGCTGTGCTGGCACACTCAACACTCGCGCTGCCATCACTCTTCCGCCGTCTTCGCCG |
| | | CCATCCTCGGCGCGACTCGCTTCTTTCGGTTCTACCAGGTAGAGTCCGCCGCCATCCTCA |
| 465 | SRS069 | GGGGCGGGGtgATGACACAGCAATtcGGGACTTTCCacGCTTGCGTGAGAAGagACCGGAA |
| | | GTgaATGACACAGCAATGGATCCGCTTGCGTGAGAAGctGGGACTTTCCtaGGGCGGGGGt |
| | | tGGGACTTTCCacATGACACAGCAATacCTCGAGGGTGACTCATGATGATGCCACGTCACC |
| | | AATGCCACGTCACCAGGTGACTCATGGGTGACTCATGaCgTgTgAcATGCCACGTCACCAA |
| | | TGCCACGTCACCAGGTGACTCATGGGTGACTCATGGGTACCACCTCTTAACAATACGTTTC |
| | | ACAAATAGTTAAAAACATGCATACTGAAAAGCATACTTTTGCAATGTTATTTTTAAAAACA |
| | | AGGAACTCTTTAACCCAGGGAAGATAATCACTTGGGGAAAGGAAGGTTCGTTTCTGAGTTA |
| | | GCAACAAGTAAATGCAGCACTAGTGGGTGGGATTGAGGTgTGCCCTGGTGCATAAATAGAG |
| | | ACTCAGCTGTGCTGGCACACTCAACtttttccgtgctacctgcagaggggtccatacggcg |
| | | ttgttctggattca |
| 466 | SRS070 | GGGGCGGGGtgATGACACAGCAATtcGGGACTTTCCacGCTTGCGTGAGAAGagACCGGAA |
| | | GTgaATGACACAGCAATGGATCCGCTTGCGTGAGAAGctGGGACTTTCCtaGGGGCGGGGt |
| | | tGGGACTTTCCacATGACACAGCAATacCTCGAGGGTGACTCATGATGATGCCACGTCACC |
| | | AATGCCACGTCACCAGGTGACTCATGGGTGACTCATGaCgTgTgAcATGCCACGTCACCAA |
| | | TGCCACGTCACCAGGTGACTCATGGGTGACTCATGGGTACCACCTCTTAACAATACGTTTC |
| | | ACAAATAGTTAAAAACATGCATACTGAAAAGCATACTTTTGCAATGTTATTTTTAAAAACA |
| | | AGGAACTCTTTAACCCAGGGAAGATAATCACTTGGGGAAAGGAAGGTTCGTTTCTGAGTTA |
| | | GCAACAAGTAAATGCAGCACTAGTGGGTGGGATTGAGGTgTGCCCTGGTGCATAAATAGAG |
| | | ACTCAGCTGTGCTGGCACACTCAAcggcggcgcagatcgcccggcgcggctccgccccctg |
| | | cgccggtcacgtgggggcgccggctgcgcctgcggagaagcggtggccgccgagcgggatc |
| | | tgtgcggggagccggaaatggttgtggactacgtctgtgcggctgcgtggggctcggccgc |
| | | gcggactgaaggagactgaaggtgctggggggaccctgatgtggA |
| 467 | SRS071 | GGGGCGGGGtgATGACACAGCAATtcGGGACTTTCCacGCTTGCGTGAGAAGagACCGGAA |
| | | GTgaATGACACAGCAATGGATCCGCTTGCGTGAGAAGctGGGACTTTCCtaGGGGCGGGGt |
| | | tGGGACTTTCCacATGACACAGCAATacCTCGAGGGTGACTCATGATGATGCCACGTCACC |
| | | AATGCCACGTCACCAGGTGACTCATGGGTGACTCATGaCgTgTgAcATGCCACGTCACCAA |
| | | TGCCACGTCACCAGGTGACTCATGGGTGACTCATGGGTACCGGGAAAAGTTCAGCTGAGAG |
| | | ATATAAAAGAGCAGTCTTTCCAGCACCTGCGAAGCTTGGACCGCATCCTAGCCGCCGACTC |
| | | ACACAAGGCAGGTGGGTGAGGAAATCCAGGTAAGGCTCCTGACAGCAGCTTTAGAAGGGTA |
| | | CTTGCTGGAGTGAATTCGGGCCTCTGATTA |
| 468 | SRS072 | GGGGCGGGGTGATGACACAGCAATTCGGGACTTTCCACGCTTGCGTGAGAAGAGACCGGAA |
| | | GTGAATGACACAGCAATGGATCCGCTTGCGTGAGAAGCTGGGACTTTCCTAGGGGCGGGGT |
| | | TGGGACTTTCCACATGACACAGCAATACCTCGAGGGTGACTCATGATGATGCCACGTCACC |
| | | AATGCCACGTCACCAGGTGACTCATGGGTGACTCATGACGTGTGACATGCCACGTCACCAA |
| | | TGCCACGTCACCAGGTGACTCATGGGTGACTCATGGGTACCGGGAAAAGTTCAGCTGAGAG |
| | | ATATAAAAGAGCAGTCTTTCCAGCACCTGCGTATCCCAGGAGGAGCAAGTGGCACGTCTTC |
| | | GGGTGAGTGTGCGGCTGTGCTGGAGCCCGGGTTACCAGCTCTTAA |
| 469 | SRS073 | GGGGCGGGGTGATGACACAGCAATTCGGGACTTTCCACGCTTGCGTGAGAAGAGACCGGAA |
| | | GTGAATGACACAGCAATGGATCCGCTTGCGTGAGAAGCTGGGACTTTCCTAGGGGCGGGGT |
| | | TGGGACTTTCCACATGACACAGCAATACCTCGAGGGTGACTCATGATGATGCCACGTCACC |
| | | AATGCCACGTCACCAGGTGACTCATGGGTGACTCATGACGTGTGACATGCCACGTCACCAA |
| | | TGCCACGTCACCAGGTGACTCATGGGTGACTCATGGGTACCGGGAAAAGTTCAGCTGAGAG |
| | | ATATAAAAGAGCAGTCTTTCCAGCACCTGCCACTCGCGCTGCCATCACTCTTCCGCCGTCT |
| | | TCGCCGCCATCCTCGGCGCGACTCGCTTCTTTCGGTTCTACCAGGTAGAGTCCGCCGCCAT |
| | | CCTCA |
| 470 | SRS074 | GGGGCGGGGtgATGACACAGCAATtcGGGACTTTCCacGCTTGCGTGAGAAGagACCGGAA |
| | | GTgaATGACACAGCAATGGATCCGCTTGCGTGAGAAGctGGGACTTTCCtaGGGGCGGGGt |
| | | tGGGACTTTCCacATGACACAGCAATacCTCGAGGGTGACTCATGATGATGCCACGTCACC |
| | | AATGCCACGTCACCAGGTGACTCATGGGTGACTCATGaCgTgTgAcATGCCACGTCACCAA |
| | | TGCCACGTCACCAGGTGACTCATGGGTGACTCATGGGTACCGGGAAAAGTTCAGCTGAGAG |
| | | ATATAAAAGAGCAGTCTTTCCAGCACCTGCCtttttccgtgctacctgcagaggggtccat |
| | | acggcgttgttctggattc |
| 471 | SRS075 | GGGGCGGGGtgATGACACAGCAATtcGGGACTTTCCacGCTTGCGTGAGAAGagACCGGAA |
| | | GTgaATGACACAGCAATGGATCCGCTTGCGTGAGAAGctGGGACTTTCCtaGGGGCGGGGt |
| | | tGGGACTTTCCacATGACACAGCAATacCTCGAGGGTGACTCATGATGATGCCACGTCACC |
| | | AATGCCACGTCACCAGGTGACTCATGGGTGACTCATGaCgTgTgAcATGCCACGTCACCAA |
| | | TGCCACGTCACCAGGTGACTCATGGGTGACTCATGGGTACCGGGAAAAGTTCAGCTGAGAG |
| | | ATATAAAAGAGCAGTCTTTCCAGCACCTGCcggggcgcagatcgcccggcgcggctccgc |
| | | cccctgcgccggtcacgtgggggcgccggctgcgcctgcggagaagcggtggccgccgagc |
| | | gggatctgtgcggggagccggaaatggttgtggactacgtctgtgcggctgcgtggggctc |
| | | ggccgcgcggactgaaggagactgaaggtgctggggggaccctgatgtgg |
| 472 | SRS076 | GGGGCGGGGtgATGACACAGCAATtcGGGACTTTCCacGCTTGCGTGAGAAGagACCGGAA |
| | | GTgaATGACACAGCAATGGATCCGCTTGCGTGAGAAGctGGGACTTTCCtaGGGGCGGGGt |
| | | tGGGACTTTCCacATGACACAGCAATacCTCGAGGGTGACTCATGATGATGCCACGTCACC |
| | | AATGCCACGTCACCAGGTGACTCATGGGTGACTCATGaCgTgTgAcATGCCACGTCACCAA |
| | | TGCCACGTCACCAGGTGACTCATGGGTGACTCATGGGTACCCGGCCCGCCCCCTTTCCTTA |
| | | CGCGGATTGGTAGCTGCAGGCTTCCCTATCTGATTGGCCGAACGAACGCAGCGCGTAATTT |
| | | AAAATATTGTATCTGTAACAAAGCTGCACCTCGTGGGCGGAGTTGTGCTCTGCGGCTGCGA |
| | | AAGTCCAGCTTCGGCGACTAGGTGTGAGTAAGCCAAAATCCAGAGCGGCGGGCACTGACGG |
| | | GCACTTGCACCGTGTGGACAGACTCTCCGGTTCTGTGAGTGGTTTTTCTTTTCCCGGGTCG |
| | | GACCTGGAGTTCTTAGGGGGATGGCTGAAgaattc |
| 473 | SRS077 | GGGGCGGGGtgATGACACAGCAATtcGGGACTTTCCacGCTTGCGTGAGAAGagACCGGAA |
| | | GTgaATGACACAGCAATGGATCCGCTTGCGTGAGAAGctGGGACTTTCCtaGGGGCGGGGt |
| | | tGGGACTTTCCacATGACACAGCAATacCTCGAGGGTGACTCATGATGATGCCACGTCACC |
| | | AATGCCACGTCACCAGGTGACTCATGGGTGACTCATGaCgTgTgAcATGCCACGTCACCAA |
| | | TGCCACGTCACCAGGTGACTCATGGGTGACTCATGGGTACCCGGCCCGCCCCCTTTCCTTA |
| | | CGCGGATTGGTAGCTGCAGGCTTCCCTATCTGATTGGCCGAACGAACGCAGCGCGTAATTT |
| | | AAAATATTGTATCTGTAACAAAGCTGCACCTCGTGGGCGGAGTTGTGCTCTGCGGCTGCGA |
| | | AAGTCCAGCTTCGGCGACTAGGTGTGAGTAAGCCAGAAGCTTGGACCGCATCCTAGCCGCC |
| | | GACTCACACAAGGCAGGTGGGTGAGGAAATCCAGGTAAGGCTCCTGACAGCAGCTTTAGAA |
| | | GGGTACTTGCTGGAGTGAATTCGGGCCTCTGATT |
| 474 | SRS078 | GGGGCGGGGtgATGACACAGCAATtcGGGACTTTCCacGCTTGCGTGAGAAGagACCGGAA |
| | | GTgaATGACACAGCAATGGATCCGCTTGCGTGAGAAGctGGGACTTTCCtaGGGGCGGGGt |
| | | tGGGACTTTCCacATGACACAGCAATacCTCGAGGGTGACTCATGATGATGCCACGTCACC |
| | | AATGCCACGTCACCAGGTGACTCATGGGTGACTCATGaCgTgTgAcATGCCACGTCACCAA |
| | | TGCCACGTCACCAGGTGACTCATGGGTGACTCATGGGTACCCGGCCCGCCCCCTTTCCTTA |
| | | CGCGGATTGGTAGCTGCAGGCTTCCCTATCTGATTGGCCGAACGAACGCAGCGCGTAATTT |
| | | AAAATATTGTATCTGTAACAAAGCTGCACCTCGTGGGCGGAGTTGTGCTCTGCGGCTGCGA |
| | | AAGTCCAGCTTCGGCGACTAGGTGTGAGTAAGCCACACTCGCGCTGCCATCACTCTTCCGC |
| | | CGTCTTCGCCGCCATCCTCGGCGCGACTCGCTTCTTTCGGTTCTACCAGGTAGAGTCCGCC |
| | | GCCATCCTC |
| 475 | SRS079 | GGGGCGGGGtgATGACACAGCAATtcGGGACTTTCCacGCTTGCGTGAGAAGagACCGGAA |
| | | GTgaATGACACAGCAATGGATCCGCTTGCGTGAGAAGctGGGACTTTCCtaGGGCGGGGGt |
| | | tGGGACTTTCCacATGACACAGCAATacCTCGAGGGTGACTCATGATGATGCCACGTCACC |
| | | AATGCCACGTCACCAGGTGACTCATGGGTGACTCATGaCqTqTqAcATGCCACGTCACCAA |
| | | TGCCACGTCACCAGGTGACTCATGGGTGACTCATGGGTACCCGGCCCGCCCCCTTTCCTTA |
| | | CGCGGATTGGTAGCTGCAGGCTTCCCTATCTGATTGGCCGAACGAACGCAGCGCGTAATTT |
| | | AAAATATTGTATCTGTAACAAAGCTGCACCTCGTGGGCGGAGTTGTGCTCTGCGGCTGCGA |
| | | AAGTCCAGCTTCGGCGACTAGGTGTGAGTAAGCCACtttttccgtgctacctgcagagggg |
| | | tccatacggcgttgttctggattc |
| 476 | SRS080 | GGGGCGGGGtgATGACACAGCAATtcGGGACTTTCCacGCTTGCGTGAGAAGagACCGGAA |
| | | GTgaATGACACAGCAATGGATCCGCTTGCGTGAGAAGctGGGACTTTCCtaGGGGCGGGGt |
| | | tGGGACTTTCCacATGACACAGCAATacCTCGAGGGTGACTCATGATGATGCCACGTCACC |
| | | AATGCCACGTCACCAGGTGACTCATGGGTGACTCATGaCgTgTgAcATGCCACGTCACCAA |
| | | TGCCACGTCACCAGGTGACTCATGGGTGACTCATGGGTACCCGGCCCGCCCCCTTTCCTTA |
| | | CGCGGATTGGTAGCTGCAGGCTTCCCTATCTGATTGGCCGAACGAACGCAGCGCGTAATTT |
| | | AAAATATTGTATCTGTAACAAAGCTGCACCTCGTGGGCGGAGTTGTGCTCTGCGGCTGCGA |
| | | AAGTCCAGCTTCGGCGACTAGGTGTGAGTAAGCCAcggcggcgcagatcgcccggcgcggc |
| | | tccgccccctgcgccggtcacgtgggggcgccggctgcgcctgcggagaagcggtggccgc |
| | | cgagcgggatctgtgcggggagccggaaatggttgtggactacgtctgtgcggctgcgtgg |
| | | ggctcggccgcgcggactgaaggagactgaaggtgctggggggaccctgatgtgg |
| 477 | SRS081 | AGTATAGTGCACAGTGACTGCAGCAGGGTGACTCATGATGATGCCACGTCACCAATGCCAC |
| | | GTCACCAGGTGACTCATGGGTGACTCATGATGCCACGTCACCAATGCCACGTCACCAGGTG |
| | | ACTCATGGGTGACTCATGGGTACCTATAAAAGGCCAGCAGCAGCCTGACCACATCTCATCC |
| 478 | SRS082 | TCCTTTGATGTACGCAACTCCTTTGATGTCTATGCGTCCTTTGATGTTAAGGATTCCTTTG |
| | | ATGTAGGTACATCCTTTGATGTCCGTAAATCCTTTGATGTAAGCTTAACTCGCAATCTAGC |
| | | ATCGTCCGACGCAACGCCTTACACCATCAGAATCTGCTAGCGGTGACTCATGGGTGACTCA |
| | | TGGGTGACTCATGGGTGACTCATGCTaCqTGGTGACTCATGGGTGACTCATGGGTGACTCA |
| | | TGGGTGACTCATGGGTGACTCATGGGTACCGGGAAAAGTTCAGCTGAGAGATATAAAAGAG |
| | | CAGTCTTTCCAGCACCTGCAAATCCAGAGCGGCGGGCACTGACGGGCACTTGCACCGTGTG |
| | | GACAGACTCTCCGGTTCTGTGAGTGGTTTTTCTTTTCCCGGGTCGGACCTGGAGTTCTTAG |
| | | GGGGATGGCTGa |
| 479 | SRS083 | TCCTTTGATGTACGCAACTCCTTTGATGTCTATGCGTCCTTTGATGTTAAGGATTCCTTTG |
| | | ATGTAGGTACATCCTTTGATGTCCGTAAATCCTTTGATGTAAGCTTGGTACAACTTCTCAC |
| | | GGAGGCTTCTAACTCGCAATCTAGCATCGTCCGACGCAACGCCTTACACCATCAGAATCTG |
| | | CTAGCGGTGACTCATGGGTGACTCATGGGTGACTCATGGGTGACTCATGCTaCgTGGTGAC |
| | | TCATGGGTGACTCATGGGTGACTCATGGGTGACTCATGGGTGACTCATGGGTACCGGGAAA |
| | | AGTTCAGCTGAGAGATATAAAAGAGCAGTCTTTCCAGCACCTGCAAATCCAGAGCGGCGGG |
| | | CACTGACGGGCACTTGCACCGTGTGGACAGACTCTCCGGTTCTGTGAGTGGTTTTTCTTTT |
| | | CCCGGGTCGGACCTGGAGTTCTTAGGGGGATGGCTGa |
| 480 | SRS084 | TCCTTTGATGTACGCAACTCCTTTGATGTCTATGCGTCCTTTGATGTTAAGGATTCCTTTG |
| | | ATGTAGGTACATCCTTTGATGTCCGTAAATCCTTTGATGTGACgattcttgatatcctcga |
| | | ggctagcATGATCACCATGAGTCACCCATGAGTCACCCATGAGTCACCCATGAGTCACCCA |
| | | TGAGTCACCCATGAGTCACCCATGAGTCACCCATGAGTCACCCATGAGTCACCACTAGTGG |
| | | TACCACCTCTTAACAATACGTTTCACAAATAGTTAAAAACATGCATACTGAAAAGCATACT |
| | | TTTGCAATGTTATTTTTAAAAACAAGGAACTCTTTAACCCAGGGAAGATAATCACTTGGGG |
| | | AAAGGAAGGTTCGTTTCTGAGTTAGCAACAAGTAAATGCAGCACTAGTGGGTGGGATTGAG |
| | | GTgTGCCCTGGTGCATAAATAGAGACTCAGCTGTGCTGGCACACTCAGAAGCTTGGACCGC |
| | | ATCCTAGCCGCCGACTCACACAAGGCAGGTGGGTGAGGAAATCCAGGTAAGGCTCCTGACA |
| | | GCAGCTTTAGAAGGGTACTTGCTGGAGTGAATTCGGGCCTCTGATTA |
| 481 | SRS085 | TCCTTTGATGTACGCAACTCCTTTGATGTCTATGCGTCCTTTGATGTTAAGGATTCCTTTG |
| | | ATGTAGGTACATCCTTTGATGTCCGTAAATCCTTTGATGTGACgatcttgatatcctcgag |
| | | gctagcATGATCACCATGAGTCACCCATGAGTCACCCATGAGTCACCCATGAGTCACCCAT |
| | | GAGTCACCCATGAGTCACCCATGAGTCACCCATGAGTCACCCATGAGTCACCACTAGTGGT |
| | | ACCACCTCTTAACAATACGTTTCACAAATAGTTAAAAACATGCAtACTAGTGGGCGGGGGt |
| | | gATGACACAGCAATtcGGGACTTTCCacGCTTGCGTGAGAAGagACCGGAAGTgaATGACA |
| | | CAGCAATtcGCTTGCGTGAGAAGctGGGACTTTCCtaGGGGCGGGGttGGGACTTTCCacA |
| | | TGACACAGCAATacacTAGTAACATTTCTCTGGCCTAACTGGCCGGTACCGGGAAAAGTTC |
| | | AGCTGAGAGATATAAAAGAGCAGTCTTTCCAGCACCTGCAAATCCAGAGCGGCGGGCACTG |
| | | ACGGGCACTTGCACCGTGTGGACAGACTCTCCGGTTCTGTGAGTGGTTTTTCTTTTCCCGG |
| | | GTCGGACCTGGAGTTCTTAGGGGGATGGCTG |
| 482 | SRS086 | TCCTTTGATGTACGCAACTCCTTTGATGTCTATGCGTCCTTTGATGTTAAGGATTCCTTTG |
| | | ATGTAGGTACATCCTTTGATGTCCGTAAATCCTTTGATGTGACgatcttgatatcctcgag |
| | | gctagcATGATCACCATGAGTCACCCATGAGTCACCCATGAGTCACCCATGAGTCACCCAT |
| | | GAGTCACCCATGAGTCACCCATGAGTCACCCATGAGTCACCCATGAGTCACCACTAGTGGT |
| | | ACCACCTCTTAACAATACGTTTCACAAATAGTTAAAAACATGCAtACTAGTGGGGCGGGGt |
| | | gATGACACAGCAATtcGGGACTTTCCacGCTTGCGTGAGAAGagACCGGAAGTgaATGACA |
| | | CAGCAATtcGCTTGCGTGAGAAGctGGGACTTTCCtaGGGGCGGGGttGGGACTTTCCacA |
| | | TGACACAGCAATacacTAGTAACATTTCTCTGGCCTAACTGGCCGGTACCGGGAAAAGTTC |
| | | AGCTGAGAGATATAAAAGAGCAGTCTTTCCAGCACCTGCAAATCCAGAGCGGCGGGCACTG |
| | | ACGGGCACTTGCACCGTGTGGACAGACTCTCCGGTTCTGTGAGTGGTTTTTCTTTTCCCGG |
| | | GTCGGACCTGGAGTTCTTAGGGGGATGGCTGAA |
| 483 | SRS087 | ATGACTCAGCAATTAGCGAGTTAGAATGACTCAGCAATTATGCGTCGGACATGACTCAGCA |
| | | ATTACATCTCGATTATGACTCAGCAATTAGGATAGGCATATGACTCAGCAATTACATAGCA |
| | | GCAATGACTCAGCAATTAGCTAGTAAGCTTGGGGCGGGGtgATGACACAGCAATtcGGGAC |
| | | TTTCCacGCTTGCGTGAGAAGagACCGGAAGTgaATGACACAGCAATGGATCCGCTTGCGT |
| | | GAGAAGctGGGACTTTCCtaGGGGCGGGGttGGGACTTTCCacATGACACAGCAATacCTC |
| | | GAGGGTACcatgcataCTAGTCTGAGCGACAGTATAGTGCACAGTGACTGCAGCAGTCATT |
| | | CCTTTGATGTACGCAACTCCTTTGATGTCTATGCGTCCTTTGATGTTAAGGATTCCTTTGA |
| | | TGTAGGTACATCCTTTGATGTCCGTAAATCCTTTGATGTGACGTCTACGTATCTACCTGAT |
| | | CAAACATGCCCGGACATGTCGTAAGACATAAACATGCCCGGACATGTCCTCGCAATCTAAC |
| | | ATGCCCGGACATGTCCTCGCAATCTAACATGCCCGGACATGTCTGCAAGCTACAACATGCC |
| | | CGGACATGTCTACAATATACGTATCTACCTGATCAAACATGCCCGGACATGTCGTAAGACA |
| | | TAAACATGCCCGGACATGTCCTCGCAATCTAACATGCCCGGACATGTCCTCGCAATCTAAC |
| | | ATGCCCGGACATGTCTGCAAGCTACAACATGCCCGGACATGTCTACGTACATACTGAAAAG |
| | | CATACTTTTGCAATGTTATTTTTAAAAACAAGGAACTCTTTAACCCAGGGAAGATAATCAC |
| | | TTGGGGAAAGGAAGGTTCGTTTCTGAGTTAGCAACAAGTAAATGCAGCACTAGTGGGTGGG |
| | | ATTGAGGTgTGCCCTGGTGCATAAATAGAGACTCAGCTGTGCTGGCACACTCAGAAGCTTG |
| | | GACCGCATCCTAGCCGCCGACTCACACAAGGCAGGTGGGTGAGGAAATCCAGGTAAGGCTC |
| | | CTGACAGCAGCTTTAGAAGGGTACTTGCTGGAGTGAATTCGGGCCTCTGATTA |
| 484 | SRS088 | ATGACTCAGCAATTAGCGAGTTAGAATGACTCAGCAATTATGCGTCGGACATGACTCAGCA |
| | | ATTACATCTCGATTATGACTCAGCAATTAGGATAGGCATATGACTCAGCAATTACATAGCA |
| | | GCAATGACTCAGCAATTAGCTAGTAAGCTTGGGGCGGGGtgATGACACAGCAATtcGGGAC |
| | | TTTCCacGCTTGCGTGAGAAGagACCGGAAGTgaATGACACAGCAATGGATCCGCTTGCGT |
| | | GAGAAGctGGGACTTTCCtaGGGGCGGGGttGGGACTTTCCacATGACACAGCAATacCTC |
| | | GAGGGTACCACTAGTGTCATCTCTTTGAATATTCTGTAGTTTGAGGAGAATATTTGTTATA |
| | | TTGCACAATAAAATAAGTTTGCAAGTTTTTTTTTTCTGCCCCAAAGAGCTCTGTGTCCTTG |
| | | AACATAAAATACAAATAACCGCTATGCTGTTAATTATTAACAAATGTCCCATTTTCAACCT |
| | | AAGGAAATACCATAAAGTAACAGATATACCAACAAAAGGTTAATAATTAACAGGCATTGCC |
| | | TGAAAAGAGTATAAAAGGCTTTCAGCATGATTTTCCATATTGTGCTTCCACCACTGCCAAT |
| | | AACAAA |
| 485 | SRS089 | ATGACTCAGCAATTAGCGAGTTAGAATGACTCAGCAATTATGCGTCGGACATGACTCAGCA |
| | | ATTACATCTCGATTATGACTCAGCAATTAGGATAGGCATATGACTCAGCAATTACATAGCA |
| | | GCAATGACTCAGCAATTAGCTAGTAAGCTTGGGGCGGGGtgATGACACAGCAATtcGGGAC |
| | | TTTCCacGCTTGCGTGAGAAGagACCGGAAGTgaATGACACAGCAATGGATCCGCTTGCGT |
| | | GAGAAGctGGGACTTTCCtaGGGGCGGGGttGGGACTTTCCacATGACACAGCAATacCTC |
| | | GAGGGTACcagcttgcatgcctgcaggtcggagtactgtcctccgagcggagtactgtcct |
| | | ccgagcggagtactgtcctccgagcggagtactgtcctccgagcggagtactgtcctccga |
| | | gcggagactctagagggtatataatggatcc |
| 486 | SRS090 | TCTGTAGTTTGAGGAGAATATTTGTTATATTGCACAATAAAATAAGTTTGCAAGTTTTTTT |
| | | TTTCTGCCCCAAAGAGCTCTGTGTCCTTGAACATAAAATACAAATAACCGCTATGCTGTTA |
| | | ATTATTAACAAATGTCCCATTTTCAACCTAAGGAAATACCATAAAGTAACAGATATACCAA |
| | | CAAAAGGTTAATAATTAACAGGCATTGCCTGAAAAGAGTATAAAAGGCTTTCAGCATGATT |
| | | TTCCATATTGTGCTTCCACCACTGCCAATAACAAA |
| 556 | SRS091 | GGTGACTCATGATGATGCCACGTCACCAATGCCACGTCACCAGGTGACTCATGGGTGACTC |
| | | ATGACGTGTGACATGCCACGTCACCAATGCCACGTCACCAGGTGACTCATGGGTGACTCAT |
| | | GACTAGTGAATTCTAATTGCTGAGTCATTGCTGCTATGTAATTGCTGAGTCATATGCCTAT |
| | | CCTAATTGCTGAGTCATAATCGAGATGTAATTGCTGAGTCATGTCCGACGCATAATTGCTG |
| | | AGTCATTCTAACTCGCTAATTGCTGAGTCATGTCGACACTAGTAAGCTTGGGGCGGGGTGA |
| | | TGACACAGCAATTCGGGACTTTCCACGCTTGCGTGAGAAGAGACCGGAAGTGAATGACACA |
| | | GCAATGGATCCGCTTGCGTGAGAAGCTGGGACTTTCCTAGGGGCGGGGTTGGGACTTTCCA |
| | | CATGACACAGCAATACCTCGAGGGTACCGGGAAAAGTTCAGCTGAGAGATATAAAAGAGCA |
| | | GTCTTTCCAGCACCTGCGTATCCCAGGAGGAGCAAGTGGCACGTCTTCGGGTGAGTGTGCG |
| | | GCTGTGCTGGAGCCCGGGTTACCAGCTCTTAA |
| 557 | SRS092 | GGTGACTCATGATGATGCCACGTCACCAATGCCACGTCACCAGGTGACTCATGGGTGACTC |
| | | ATGACGTGTGACATGCCACGTCACCAATGCCACGTCACCAGGTGACTCATGGGTGACTCAT |
| | | GACTAGTGAATTCTAATTGCTGAGTCATTGCTGCTATGTAATTGCTGAGTCATATGCCTAT |
| | | CCTAATTGCTGAGTCATAATCGAGATGTAATTGCTGAGTCATGTCCGACGCATAATTGCTG |
| | | AGTCATTCTAACTCGCTAATTGCTGAGTCATGTCGACACTAGTAAGCTTGGGGCGGGGTGA |
| | | TGACACAGCAATTCGGGACTTTCCACGCTTGCGTGAGAAGAGACCGGAAGTGAATGACACA |
| | | GCAATGGATCCGCTTGCGTGAGAAGCTGGGACTTTCCTAGGGGCGGGGTTGGGACTTTCCA |
| | | CATGACACAGCAATACCTCGAGGGTACGGCCCGCCCCCTTTCCTTACGCGGATTGGTAGCT |
| | | GCAGGCTTCCCTATCTGATTGGCCGAACGAACGCAGCGCGTAATTTAAAATATTGTATCTG |
| | | TAACAAAGCTGCACCTCGTGGGCGGAGTTGTGCTCTGCGGCTGCGAAAGTCCAGCTTCGGC |
| | | GACTAGGTGTGAGTAAGCCAGTATCCCAGGAGGAGCAAGTGGCACGTCTTCGGGTGCAGTG |
| | | TGCGGCTGTGCTGGAGCCCGGGTTACCAGCTCTT |

TABLE 1D
coreBIRC5 H1299

| | | | | | SEQ | |
|---|---|---|---|---|---|---|
| | Expression | Fold | Barcode | | ID | |
| Construct | Score | Change | Support | Motif | NO: | Spacer |
| TRPS1_v22 | 2.20 | 1.95 | 5 | TATTTTATCTTT | 129 | 7 |
| MNX1_v18 | 2.05 | 1.81 | 5 | GTCATTAT | | 7 |
| TWIST1_v3 | 1.87 | 1.66 | 5 | ATTCCAGATGTTT | 131 | 3 |
| Control-1_ | 1.64 | 1.45 | 27 | | | |
| FOSL1_v1 | | | | | | |
| HOXA1_v10 | 1.47 | 1.30 | 5 | GTCATTAC | | 7 |
| TWIST1_v4 | 1.41 | 1.25 | 5 | ATTCCAGATGTTT | 131 | 0 |
| ETV4_v2 | 1.40 | 1.24 | 6 | ACCGGAAGTG | 132 | 7 |
| GATA1_v1 | 1.39 | 1.23 | 6 | TTCTAATCTAT | 133 | 10 |
| ETV4_v14 | 1.38 | 1.22 | 6 | ACCGGAAATG | 134 | 7 |
| FOSL2_v1 | 1.37 | 1.21 | 5 | GGATGACTCAT | 135 | 10 |
| NFIC_v15 | 1.33 | 1.18 | 6 | TTCTTGGCAGA | 136 | 3 |
| EN2_v7 | 1.33 | 1.18 | 5 | CGCAATTA | | 3 |
| ETV4_v6 | 1.33 | 1.18 | 6 | ACCGGAAGCG | 137 | 7 |
| SOX11_v2 | 1.32 | 1.17 | 6 | GAGAACAAAGGA | 138 | 7 |
| ETV6_v6 | 1.32 | 1.17 | 5 | ACCGGAAGTG | 132 | 7 |
| TRPS1_v20 | 1.31 | 1.16 | 6 | TAACTTATCTTT | 139 | 0 |
| TFDP1_v6 | 1.31 | 1.16 | 6 | GGGCGGGAACG | 140 | 7 |
| TCF7_v9 | 1.30 | 1.15 | 5 | TCCTTTGATAT | 141 | 10 |
| TRPS1_v10 | 1.29 | 1.14 | 6 | TAGCTTATCTTT | 142 | 7 |
| PITX2_v22 | 1.29 | 1.14 | 5 | TTAATCCA | | 7 |
| TCF7L1_v8 | 1.26 | 1.12 | 6 | AAACATCAAAGG | 143 | 0 |
| CREB3L1_v6 | 1.25 | 1.11 | 6 | ATGCCACGTCACCA | 144 | 7 |
| E2F8_v21 | 1.24 | 1.10 | 5 | TTCGCGCTAAAA | 146 | 10 |
| ZBTB7B_v6 | 1.23 | 1.09 | 6 | GCGACCACCAAA | 192 | 7 |
| ZBTB7B_v21 | 1.23 | 1.09 | 5 | GCAACCACCGAA | 270 | 10 |
| TCF7_v23 | 1.22 | 1.08 | 6 | TCCTTTGAACT | 272 | 3 |
| HOXC10_v10 | 1.22 | 1.08 | 6 | GTCGTTAAAT | 275 | 7 |
| ETV6_v15 | 1.22 | 1.08 | 6 | AGAGGAAGTG | 276 | 3 |
| VENTX_v9 | 1.22 | 1.08 | 6 | AGCGATTAG | | 10 |
| NFIC_v1 | 1.22 | 1.08 | 6 | TACTTGGCAGA | 277 | 10 |
| NFIC_v21 | 1.21 | 1.07 | 5 | TACTTGGCAAA | 280 | 10 |
| FOXN1_v17 | 1.21 | 1.07 | 6 | AGAAGC | | 10 |
| PITX2_v24 | 1.21 | 1.07 | 5 | TTAATCCA | | 0 |
| E2F4_v7 | 1.21 | 1.07 | 6 | TTTTGGCGCCCTTT | 286 | 3 |
| TCF7_v14 | 1.20 | 1.07 | 6 | TCCTTTGATTT | 287 | 7 |
| EN2_v16 | 1.20 | 1.07 | 6 | CTCAATTA | | 0 |
| DMBX1_v19 | 1.20 | 1.06 | 6 | TGAACAGGATTAATGTA | 288 | 3 |
| CREB3L1_v18 | 1.20 | 1.06 | 5 | ATGCCACGTAATCA | 294 | 7 |
| SOX11_v7 | 1.20 | 1.06 | 6 | GAGAACAAAGAA | 295 | 3 |
| ETV6_v10 | 1.20 | 1.06 | 6 | ATCGGAAGTG | 296 | 7 |
| FOSL2_v9 | 1.20 | 1.06 | 5 | GGGTGACTCAT | 297 | 10 |
| ZBTB7B_v4 | 1.20 | 1.06 | 5 | GCGACCACCGAA | 298 | 0 |
| FOXNI_v6 | 1.19 | 1.06 | 5 | GGAAGC | | 7 |
| SIX4_v16 | 1.19 | 1.06 | 5 | GAAATCTGAGC | 299 | 0 |
| TCF7_v3 | 1.19 | 1.05 | 5 | TCCTTTGATGT | 300 | 3 |
| NFIC_v9 | 1.19 | 1.05 | 6 | TACTTGGCATA | 306 | 10 |
| ETV4_v5 | 1.19 | 1.05 | 6 | ACCGGAAGCG | 137 | 10 |
| FOSL2_v17 | 1.19 | 1.05 | 6 | GGATGACTCAC | 307 | 10 |
| ETV6_v14 | 1.19 | 1.05 | 5 | AGAGGAAGTG | 276 | 7 |
| GATA1_v13 | 1.19 | 1.05 | 6 | TTCTAATCTCT | 308 | 10 |

TABLE 1E
TATA-TSS H1299

| | | | | | SEQ | |
|---|---|---|---|---|---|---|
| | Expression | Fold | Barcode | | ID | |
| Construct | Score | Change | Support | Motif | NO: | Spacer |
| Control-1_FOSL1_v1 | 3.19 | 4.84 | 27 | | | |
| FOSL2_v4 | 2.22 | 3.37 | 5 | GGATGACTCAT | 135 | 0 |
| CREB3L1_v18 | 1.87 | 2.85 | 5 | ATGCCACGTAATCA | 294 | 7 |
| Control-1_FOSL1_v2 | 1.52 | 2.31 | 24 | | | |
| FOSL2_v22 | 1.46 | 2.22 | 6 | GGGTGACTCAC | 309 | 7 |
| CREB3L1_v6 | 1.46 | 2.22 | 6 | ATGCCACGTCACCA | 144 | 7 |
| FOSL2_v17 | 1.35 | 2.04 | 6 | GGATGACTCAC | 307 | 10 |
| Control-1_ | 1.32 | 2.00 | 26 | | | |
| FOSL1_v3 | | | | | | |
| FOSL2_v7 | 1.28 | 1.94 | 6 | GGATGACTCAG | 313 | 3 |
| FOSL2_v1 | 1.28 | 1.94 | 6 | GGATGACTCAT | 135 | 10 |
| NPAS2_v11 | 1.21 | 1.84 | 6 | GACACGTGTC | 314 | 3 |
| FOSL2_v11 | 1.20 | 1.82 | 5 | GGGTGACTCAT | 297 | 3 |
| HES6_v11 | 1.11 | 1.69 | 6 | GGCACGTGTA | 316 | 3 |
| HES6_v7 | 1.09 | 1.66 | 5 | GGCACGTGTC | 317 | 3 |
| CREB3L1_v14 | 1.03 | 1.57 | 6 | ATGCCACGTCAACA | 320 | 7 |
| HES6_v3 | 0.98 | 1.49 | 6 | GGCACGTGTT | 321 | 3 |
| ASCL1_v23 | 0.96 | 1.45 | 5 | GGCACGTGCC | 322 | 3 |
| TWIST1_v3 | 0.95 | 1.43 | 5 | ATTCCAGATGTTT | 131 | 3 |
| FOSL2_v8 | 0.94 | 1.43 | 5 | GGATGACTCAG | 313 | 0 |
| TRPS1_v22 | 0.92 | 1.40 | 5 | TATTTTATCTTT | 129 | 7 |
| GRHL1_v10 | 0.90 | 1.36 | 6 | AAAACCGGTTCT | 323 | 7 |
| FOSL2_v9 | 0.87 | 1.32 | 6 | GGGTGACTCAT | 297 | 10 |
| ETV4_v14 | 0.83 | 1.27 | 6 | ACCGGAAATG | 134 | 7 |
| TWIST1_v2 | 0.82 | 1.25 | 6 | ATTCCAGATGTTT | 131 | 7 |
| SOX11_v2 | 0.82 | 1.24 | 6 | GAGAACAAAGGA | 138 | 7 |
| ZNF354A_v15 | 0.80 | 1.21 | 5 | ATAAATAAAAATG | 327 | 3 |
| | | | | GACTAATT | | |
| ZBTB7B_v4 | 0.79 | 1.20 | 5 | GCGACCACCGAA | 298 | 0 |
| ZBTB7B_v21 | 0.78 | 1.18 | 5 | GCAACCACCGAA | 270 | 10 |
| ETV6_v6 | 0.78 | 1.18 | 5 | ACCGGAAGTG | 132 | 7 |
| ETV4_v12 | 0.77 | 1.18 | 5 | ACCGGATGTG | 336 | 0 |
| ETV4_v6 | 0.77 | 1.17 | 6 | ACCGGAAGCG | 137 | 7 |
| TFDP1_v21 | 0.76 | 1.16 | 6 | GGGCGGGACCG | 337 | 10 |
| SOX11_v7 | 0.76 | 1.15 | 6 | GAGAACAAAGAA | 295 | 3 |
| FOSL2_v18 | 0.75 | 1.14 | 6 | GGATGACTCAC | 307 | 7 |
| ETV6_v10 | 0.74 | 1.13 | 6 | ATCGGAAGTG | 296 | 7 |
| FOSL2_v14 | 0.74 | 1.12 | 6 | GGGTGACTCAG | 338 | 7 |
| NFIC_v2 | 0.74 | 1.12 | 5 | TACTTGGCAGA | 277 | 7 |
| MGA_v17 | 0.73 | 1.11 | 5 | AGGTGCGA | | 10 |
| TRPS1_v20 | 0.73 | 1.11 | 6 | TAACTTATCTTT | 139 | 0 |
| IRF6_v23 | 0.73 | 1.10 | 6 | GCCGATACT | | 3 |
| ETV4_v10 | 0.72 | 1.10 | 5 | ACCGGATGTG | 336 | 7 |
| ETV4_v7 | 0.72 | 1.10 | 6 | ACCGGAAGCG | 137 | 3 |
| ZBTB7B_v24 | 0.72 | 1.09 | 6 | GCAACCACCGAA | 270 | 0 |
| SIX2_v17 | 0.72 | 1.09 | 6 | AACTGAAACTTGATAC | 339 | 10 |
| TWIST1_v23 | 0.72 | 1.09 | 6 | ATTGCAGATGTTT | 340 | 3 |
| SIX2_v5 | 0.71 | 1.08 | 5 | AACTGTAACCTGATAC | 341 | 10 |
| ETV4_v2 | 0.71 | 1.08 | 6 | ACCGGAAGTG | 132 | 7 |
| E2F7_v3 | 0.71 | 1.08 | 5 | TTTTCCCGCCAAAA | 487 | 3 |
| CUX1_v21 | 0.71 | 1.07 | 5 | TGATCAATAA | 488 | 10 |
| SIX4_v6 | 0.71 | 1.07 | 5 | GAAACATGAGC | 489 | 7 |

TABLE 1F
coreBIRC5 PDX430

| | | | | | SEQ | |
|---|---|---|---|---|---|---|
| | Expression | Fold | Barcode | | ID | |
| Construct | Score | Change | Support | Motif | NO: | Spacer |
| TCF7_v2 | 4.37 | 3.90 | 6 | TCCTTTGATGT | 300 | 7 |
| TCF7_v3 | 3.76 | 3.35 | 5 | TCCTTTGATGT | 300 | 3 |
| TCF7L1_v19 | 3.61 | 3.22 | 6 | AGACATCAAAGG | 490 | 3 |
| ETV4_v14 | 3.58 | 3.19 | 6 | ACCGGAAATG | 134 | 7 |
| TCF7L1_v5 | 3.10 | 2.76 | 6 | AAACATCAAAGG | 143 | 10 |
| TCF7L1_v8 | 3.06 | 2.73 | 6 | AAACATCAAAGG | 143 | 0 |
| ETV4_v2 | 3.01 | 2.68 | 6 | ACCGGAAGTG | 132 | 7 |
| ETV4_v6 | 2.96 | 2.64 | 6 | ACCGGAAGCG | 137 | 7 |
| ETV4_v10 | 2.92 | 2.61 | 5 | ACCGGATGTG | 336 | 7 |
| ETV4_v13 | 2.73 | 2.43 | 6 | ACCGGAAATG | 134 | 10 |
| TWIST1_v3 | 2.67 | 2.38 | 5 | ATTCCAGATGTTT | 131 | 3 |
| TCF7L_1_v24 | 2.61 | 2.33 | 6 | AAACTTCAAAGG | 491 | 0 |
| TCF7_v23 | 2.54 | 2.27 | 6 | TCCTTTGAACT | 272 | 3 |
| ETV4_v8 | 2.53 | 2.26 | 5 | ACCGGAAGCG | 137 | 0 |
| DLX1_v24 | 2.47 | 2.20 | 6 | GTCATTAC | | 0 |
| TCF7_v7 | 2.41 | 2.15 | 5 | TCCTTTGATCT | 492 | 3 |
| ETV6_v6 | 2.29 | 2.04 | 5 | ACCGGAAGTG | 132 | 7 |
| ETV4_v5 | 2.29 | 2.04 | 6 | ACCGGAAGCG | 137 | 10 |
| ETV4_v7 | 2.14 | 1.91 | 6 | ACCGGAAGCG | 137 | 3 |
| TWIST1_v2 | 2.10 | 1.88 | 6 | ATTCCAGATGTTT | 131 | 7 |
| TRPS1_v22 | 2.05 | 1.83 | 5 | TATTTTATCTTT | 129 | 7 |
| SIX2_v5 | 2.05 | 1.83 | 5 | AACTGTAACCTGATAC | 341 | 10 |
| HOXA1_v8 | 2.01 | 1.79 | 6 | GTAATGAC | | 0 |
| HOXC10_v24 | 1.97 | 1.75 | 6 | GTCGTAAACT | 493 | 0 |
| HOXA1_v12 | 1.95 | 1.74 | 6 | GTCATTAC | | 0 |
| HOXB9_v18 | 1.94 | 1.73 | 6 | GTCGTAAAGT | 494 | 7 |
| ETV4_v16 | 1.90 | 1.70 | 5 | ACCGGAAATG | 134 | 0 |
| HOXC10_v14 | 1.85 | 1.65 | 6 | GTCGTAAATT | 495 | 7 |
| ETV6_v8 | 1.84 | 1.64 | 6 | ACCGGAAGTG | 132 | 0 |
| ETV4_v1 | 1.82 | 1.63 | 6 | ACCGGAAGTG | 132 | 10 |
| MYCN_v22 | 1.80 | 1.60 | 5 | GTCCACGTGGCC | 496 | 7 |
| SP3_v8 | 1.79 | 1.59 | 5 | GGCCCCGCCCACC | 497 | 0 |
| HOXC10_v15 | 1.78 | 1.58 | 6 | GTCGTAAATT | 495 | 3 |
| TCF7_v18 | 1.72 | 1.54 | 5 | TCCTTTGAAGT | 498 | 7 |
| TCF7_v22 | 1.72 | 1.53 | 5 | TCCTTTGAACT | 272 | 7 |
| ETV4_v23 | 1.72 | 1.53 | 6 | AGCGGAAGTG | 499 | 3 |
| ZNF281_v13 | 1.71 | 1.52 | 5 | GGGGGAAGGGAG | 500 | 10 |
| HOXC10_v4 | 1.71 | 1.52 | 6 | GTCGTAAAAT | 501 | 0 |
| FOSL2_v1 | 1.70 | 1.51 | 5 | GGATGACTCAT | 135 | 10 |
| PAX8_v19 | 1.64 | 1.46 | 5 | GTCATGCATGACTGC | 502 | 3 |
| E2F2_v23 | 1.62 | 1.45 | 6 | GTTTGGGCGCCATTTC | 503 | 3 |
| SP3_v19 | 1.61 | 1.43 | 5 | GGACCCGCCCACC | 504 | 3 |
| SIX4_v4 | 1.60 | 1.43 | 5 | GAAACCTGAGC | 505 | 0 |
| SIX4_v10 | 1.58 | 1.41 | 5 | GAAACTTGAGC | 506 | 7 |
| NFIC_v10 | 1.56 | 1.39 | 5 | TACTTGGCATA | 306 | 7 |
| HOXC9_v15 | 1.56 | 1.39 | 6 | GTCGTAAACT | 493 | 3 |
| PAX7_v15 | 1.55 | 1.38 | 5 | ATTAATCGATTATTT | 507 | 3 |
| RUNX1_v17 | 1.52 | 1.36 | 5 | GTCTGTGGCTT | 508 | 10 |
| DLX1_v8 | 1.52 | 1.36 | 6 | GTAATTAC | | 0 |
| RREB1_v14 | 1.52 | 1.35 | 6 | CCCCAAACCACCACCCCC | 509 | 7 |
| | | | | CC | | |

TABLE 1G

| | | | | | SEQ | |
|---|---|---|---|---|---|---|
| | Expression | Fold | Barcode | | ID | |
| construct | Score | Change | Support | Motif | NO: | Spacer |
| TCF7_v2 | 5.12 | 11.18 | 6 | TCCTTTGATGT | 300 | 7 |
| TCF7L1_v19 | 4.35 | 9.49 | 6 | AGACATCAAAGG | 490 | 3 |
| TCF7_v7 | 3.21 | 7.00 | 5 | TCCTTTGATCT | 492 | 3 |
| TCF7_v19 | 2.78 | 6.07 | 5 | TCCTTTGAAGT | 498 | 3 |
| TCF7_v3 | 2.78 | 6.06 | 5 | TCCTTTGATGT | 300 | 3 |
| ETV4_v14 | 2.54 | 5.54 | 6 | ACCGGAAATG | 134 | 7 |
| TCF7L_1_v5 | 2.44 | 5.39 | 6 | AAACATCAAAGG | 143 | 10 |
| ETV4_v2 | 2.37 | 5.17 | 6 | ACCGGAAGTG | 132 | 7 |
| ETV4_v6 | 2.36 | 5.15 | 6 | ACCGGAAGCG | 137 | 7 |
| ETV4_v10 | 2.29 | 5.00 | 5 | ACCGGATGTG | 336 | 7 |
| ETV6_v6 | 2.18 | 4.75 | 5 | ACCGGAAGTG | 132 | 7 |
| HOXC10_v24 | 2.07 | 4.51 | 6 | GTCGTAAACT | 493 | 0 |
| HOXC10_v4 | 2.01 | 4.38 | 6 | GTCGTAAAAT | 501 | 0 |
| ETV4_v8 | 1.94 | 4.23 | 5 | ACCGGAAGCG | 137 | 0 |
| TCF7L_1_v4 | 1.91 | 4.16 | 5 | AAAGATCAAAGG | 510 | 0 |
| TCF7_v23 | 1.87 | 4.09 | 6 | TCCTTTGAACT | 272 | 3 |
| ZNF354A_v7 | 1.80 | 3.94 | 5 | ATAAATATAAAAG | 511 | 3 |
| | | | | GACTAATT | | |
| TCF7_v18 | 1.80 | 3.93 | 5 | TCCTTTGAAGT | 498 | 7 |
| TCF7L1_v11 | 1.69 | 3.70 | 6 | AGAGATCAAAGG | 512 | 3 |
| DLX1_v24 | 1.65 | 3.61 | 6 | GTCATTAC | | 0 |
| FOSL2_v4 | 1.64 | 3.58 | 5 | GGATGACTCAT | 135 | 0 |
| ZNF384_v14 | 1.63 | 3.55 | 5 | TTGAAAAAAAAA | 513 | 7 |
| HNF1A_v13 | 1.62 | 3.54 | 5 | AGTTAATTATTAACT | 514 | 10 |
| SIX4_v6 | 1.59 | 3.48 | 5 | GAAACATGAGC | 489 | 7 |
| ETV4_v13 | 1.58 | 3.46 | 6 | ACCGGAAATG | 134 | 10 |
| PAX7_v3 | 1.54 | 3.37 | 5 | ATTAATCAATTATTT | 515 | 3 |
| TCF7L1_v24 | 1.53 | 3.35 | 6 | AAACTTCAAAGG | 491 | 0 |
| SP3_v24 | 1.50 | 3.28 | 6 | GGCCCCGCCTACC | 516 | 0 |
| HOXB9_v4 | 1.47 | 3.21 | 5 | GTCGTAAAAT | 501 | 0 |
| TCF7L1_v23 | 1.44 | 3.14 | 6 | AAACTTCAAAGG | 491 | 3 |
| TCF7L_1_v8 | 1.44 | 3.13 | 6 | AAACATCAAAGG | 143 | 0 |
| E2F3_v20 | 1.43 | 3.12 | 5 | ATTTTGGCGCGAAAAT | 517 | 0 |
| HOXA1_v8 | 1.42 | 3.09 | 6 | GTAATGAC | | 0 |
| RORB_v4 | 1.38 | 3.00 | 6 | AATTAGGTCAC | 518 | 0 |
| PAX7_v12 | 1.37 | 3.00 | 5 | ATTAATCAATTTTTT | 519 | 0 |
| HOXB9_v13 | 1.37 | 2.99 | 6 | GTCGTAAACT | 493 | 10 |
| TCF7_v22 | 1.36 | 2.97 | 5 | TCCTTTGAACT | 272 | 7 |
| SP3_v12 | 1.35 | 2.95 | 6 | GGACACGCCCACC | 520 | 0 |
| HOXA1_v4 | 1.35 | 2.95 | 6 | GTAATTAC | | 0 |
| HOXB9_v17 | 1.34 | 2.92 | 6 | GTCGTAAAGT | 494 | 10 |
| HOXB9_v18 | 1.34 | 2.92 | 6 | GTCGTAAAGT | 494 | 7 |
| HOXC10_v15 | 1.33 | 2.91 | 6 | GTCGTAAATT | 495 | 3 |
| HOXC9_v15 | 1.33 | 2.91 | 6 | GTCGTAAACT | 493 | 3 |
| ETV4_v1 | 1.32 | 2.89 | 6 | ACCGGAAGTG | 132 | 10 |
| SP3_v11 | 1.32 | 2.89 | 6 | GGACACGCCCACC | 520 | 3 |
| ETV4_v19 | 1.32 | 2.88 | 5 | ACCGGAAGGG | 521 | 3 |
| ETV4_v16 | 1.32 | 2.88 | 5 | ACCGGAAATG | 134 | 0 |
| HOXC10_v14 | 1.31 | 2.87 | 6 | GTCGTAAATT | 495 | 7 |
| TWIST1_v3 | 1.31 | 2.85 | 5 | ATTCCAGATGTTT | 131 | 3 |
| DLX4_v3 | 1.29 | 2.82 | 6 | CCAATTAC | | 3 |

TABLE 1H
coreBIRC5 PDX586

| | | | | | SEQ | |
|---|---|---|---|---|---|---|
| | Expression | Fold | Barcode | | ID | |
| Construct | Score | Change | Support | Motif | NO: | Spacer |
| TRPS1_v22 | 2.22 | 1.85 | 5 | TATTTTATCTTT | 129 | 7 |
| TP53_v21 | 1.80 | 1.50 | 5 | AACATGCCTGGGCATGTC | 522 | 10 |
| TP53_v5 | 1.76 | 1.47 | 6 | AACATGCCCGGACATGTC | 523 | 10 |
| TWIST1_v3 | 1.75 | 1.46 | 5 | ATTCCAGATGTTT | 131 | 3 |
| MYCN_v13 | 1.70 | 1.42 | 5 | GCCCACGTGGCC | 524 | 10 |
| MNX1_v18 | 1.66 | 1.38 | 5 | GTCATTAT | | 7 |
| TP53_v1 | 1.65 | 1.37 | 6 | AACATGCCCGGGCATGTC | 525 | 10 |
| TP53_v10 | 1.59 | 1.32 | 5 | AACATGTCCGGGCATGTC | 526 | 7 |
| HOXB9_v5 | 1.57 | 1.31 | 6 | GTCGTAAATT | 495 | 10 |
| SIX2_v5 | 1.57 | 1.31 | 5 | AACTGTAACCTGATAC | 341 | 10 |
| TP63_v3 | 1.56 | 1.30 | 5 | AACATGTTGGGACATGTC | 527 | 3 |
| SIX4_v16 | 1.55 | 1.29 | 5 | GAAATCTGAGC | 299 | 0 |
| HOXB9_v15 | 1.51 | 1.26 | 6 | GTCGTAAACT | 493 | 3 |
| SOX11_v16 | 1.50 | 1.25 | 5 | GAGAACAAAGCA | 528 | 0 |
| E2F8_v21 | 1.50 | 1.25 | 5 | TTCGCGCTAAAA | 146 | 10 |
| HOXA1_v12 | 1.49 | 1.24 | 6 | GTCATTAC | | 0 |
| TP53_v6 | 1.48 | 1.23 | 6 | AACATGCCCGGACATGTC | 523 | 7 |
| CREB3L1_v1 | 1.46 | 1.22 | 5 | ATGCCACGTCATCA | 529 | 10 |
| TFDP1_v6 | 1.45 | 1.21 | 6 | GGGCGGGAACG | 140 | 7 |
| ETV4_v14 | 1.44 | 1.20 | 6 | ACCGGAAATG | 134 | 7 |
| SURV_v9 | 1.43 | 1.20 | 6 | GGGCGTGCGCTCCCGACA | 530 | 0 |
| | | | | AGCCC | | |
| TP53_v16 | 1.41 | 1.18 | 6 | AACATGCCCAGGCATGTC | 531 | 0 |
| TP53_v8 | 1.41 | 1.18 | 5 | AACATGCCCGGACATGTC | 523 | 0 |
| FOXE1_v3 | 1.40 | 1.17 | 5 | CCTAAATAAACAAA | 532 | 3 |
| EN1_v23 | 1.40 | 1.17 | 6 | GCAATTAG | | 3 |
| ZBTB7B_v21 | 1.40 | 1.17 | 5 | GCAACCACCGAA | 270 | 10 |
| TRPS1_v20 | 1.40 | 1.16 | 6 | TAACTTATCTTT | 139 | 0 |
| TP53_v22 | 1.39 | 1.16 | 6 | AACATGCCTGGGCATGTC | 522 | 7 |
| SP3_v8 | 1.39 | 1.16 | 5 | GGCCCCGCCCACC | 497 | 0 |
| SIX2_v20 | 1.38 | 1.15 | 5 | AACTGAAACTTGATAC | 339 | 0 |
| TP53_v7 | 1.38 | 1.15 | 5 | AACATGCCCGGACATGTC | 523 | 3 |
| TWIST1_v1 | 1.37 | 1.15 | 5 | ATTCCAGATGTTT | 131 | 10 |
| MYBL2_v4 | 1.37 | 1.15 | 5 | AACCGTTAAACGGTC | 533 | 0 |
| SIX2_v17 | 1.37 | 1.14 | 6 | AACTGAAACTTGATAC | 339 | 10 |
| TP53_v24 | 1.36 | 1.14 | 6 | AACATGCCTGGGCATGTC | 522 | 0 |
| TRPS1_v11 | 1.36 | 1.13 | 5 | TAGCTTATCTTT | 142 | 3 |
| Control-O_Filler_v3 | 1.36 | 1.13 | 26 | | | |
| TP53_v20 | 1.35 | 1.13 | 6 | AACATGTCCGGACATGTC | 534 | 0 |
| GATA1_v1 | 1.35 | 1.12 | 6 | TTCTAATCTAT | 133 | 10 |
| SHOX2_v16 | 1.34 | 1.12 | 5 | CCAATTAG | | 0 |
| TP53_v9 | 1.33 | 1.11 | 6 | AACATGTCCGGGCATGTC | 526 | 10 |
| HOXB7_v16 | 1.33 | 1.11 | 6 | GGTAATTGAC | 535 | 0 |
| E2F4_v9 | 1.32 | 1.10 | 5 | TTTTGGCGCCTTTT | 536 | 10 |
| E2F2_v12 | 1.31 | 1.09 | 5 | GTTTTGGCGCCTTTTC | 537 | 0 |
| SIX4_v21 | 1.30 | 1.09 | 5 | GAAATTTGAGC | 538 | 10 |
| SURV_v3 | 1.30 | 1.09 | 5 | GGGCAAGCGCTCCCGA | 539 | 0 |
| | | | | CATGCCC | | |
| DLX4_v12 | 1.30 | 1.08 | 6 | CAAATTAC | | 0 |
| BARX1_v11 | 1.29 | 1.08 | 6 | GCGATTAG | | 3 |
| NR2F6_v4 | 1.29 | 1.08 | 5 | GAGGTCAAAGGTCA | 540 | 0 |
| TFDP1_v7 | 1.29 | 1.07 | 5 | GGGCGGGAACG | 140 | 3 |

TABLE 1I
TATA-TSS PDX586

| | | | | | SEQ | |
|---|---|---|---|---|---|---|
| | Expression | Fold | Barcode | | ID | |
| Construct | Score | Change | Support | Motif | NO: | Spacer |
| TP53_v5 | 2.73 | 5.63 | 6 | AACATGCCCGGACATGT | 523 | 10 |
| | | | | C | | |
| NPAS2_v11 | 2.59 | 5.34 | 6 | GACACGTGTC | 314 | 3 |
| HES6_v11 | 2.52 | 5.21 | 6 | GGCACGTGTA | 316 | 3 |
| SURV_v3 | 2.41 | 4.97 | 6 | GGGCAAGCGCTCCCGAC | 539 | 0 |
| | | | | ATGCCC | | |
| TP53_v22 | 1.93 | 3.97 | 6 | AACATGCCTGGGCATGT | 522 | 7 |
| | | | | C | | |
| HES6_v3 | 1.82 | 3.76 | 6 | GGCACGTGTT | 321 | 3 |
| TP53_v10 | 1.79 | 3.69 | 6 | AACATGTCCGGGCATGT | 526 | 7 |
| | | | | C | | |
| TP53_v13 | 1.79 | 3.69 | 5 | AACATGCCCAGGCATGT | 531 | 10 |
| | | | | C | | |
| TP53_v18 | 1.74 | 3.60 | 5 | AACATGTCCGGACATGT | 534 | 7 |
| | | | | C | | |
| TP53_v16 | 1.74 | 3.59 | 6 | AACATGCCCAGGCATGT | 531 | 0 |
| | | | | C | | |
| SURV_v15 | 1.73 | 3.57 | 6 | GGGCTAGCGCTCCCGAC | 541 | 0 |
| | | | | ATGCCC | | |
| HES6_v7 | 1.71 | 3.53 | 5 | GGCACGTGTC | 317 | 3 |
| ASCL1_v23 | 1.66 | 3.43 | 5 | GGCACGTGCC | 322 | 3 |
| TFDP1_v4 | 1.59 | 3.27 | 6 | GGGCGGGAAGG | 542 | 0 |
| FOSL2_v4 | 1.57 | 3.25 | 5 | GGATGACTCAT | 135 | 0 |
| TFDP1_v19 | 1.57 | 3.23 | 5 | GGGCGGGACGG | 543 | 3 |
| TP53_v1 | 1.55 | 3.19 | 6 | AACATGCCCGGGCATGT | 525 | 10 |
| | | | | C | | |
| Control-1_ | 1.54 | 3.18 | 27 | | | |
| FOSL_1_v1 | | | | | | |
| MYC_v22 | 1.46 | 3.01 | 6 | GGACACGTGCCC | 544 | 7 |
| TP53_v6 | 1.45 | 2.99 | 6 | AACATGCCCGGACATGT | 523 | 7 |
| | | | | C | | |
| SP3_v24 | 1.45 | 2.98 | 6 | GGCCCCGCCTACC | 516 | 0 |
| CREB3L1_v18 | 1.42 | 2.92 | 5 | ATGCCACGTAATCA | 294 | 7 |
| ETV4_v10 | 1.41 | 2.90 | 5 | ACCGGATGTG | 336 | 7 |
| CREB3L1_v6 | 1.37 | 2.82 | 6 | ATGCCACGTCACCA | 144 | 7 |
| SOX11_v17 | 1.33 | 2.75 | 6 | GGGAACAAAGAA | 545 | 10 |
| SP3_v12 | 1.32 | 2.73 | 6 | GGACACGCCCACC | 520 | 0 |
| TP53_v24 | 1.31 | 2.70 | 6 | AACATGCCTGGGCATGT | 522 | 0 |
| | | | | C | | |
| SP3_v20 | 1.30 | 2.69 | 6 | GGACCCGCCCACC | 504 | 0 |
| HOXC9_v15 | 1.30 | 2.68 | 6 | GTCGTAAACT | 493 | 3 |
| ETV4_v14 | 1.28 | 2.65 | 6 | ACCGGAAATG | 134 | 7 |
| HOXC10_v14 | 1.28 | 2.64 | 6 | GTCGTAAATT | 495 | 7 |
| SP3_v22 | 1.28 | 2.64 | 5 | GGCCCCGCCTACC | 516 | 7 |
| HES6_v6 | 1.27 | 2.61 | 6 | GGCACGTGTC | 317 | 7 |
| CREB3L1_v14 | 1.26 | 2.61 | 6 | ATGCCACGTCAACA | 320 | 7 |
| SURV_v6 | 1.25 | 2.58 | 6 | GGGCATGCGCTCCCGAC | 546 | 0 |
| | | | | ATGCCC | | |
| FOSL2_v7 | 1.25 | 2.57 | 6 | GGATGACTCAG | 313 | 3 |
| HOXC10_v15 | 1.24 | 2.57 | 6 | GTCGTAAATT | 495 | 3 |
| HOXA1_v8 | 1.23 | 2.54 | 6 | GTAATGAC | | 0 |
| BARX1_v7 | 1.23 | 2.53 | 5 | GCCATTAG | | 3 |
| HES6_v10 | 1.22 | 2.51 | 5 | GGCACGTGTA | 316 | 7 |
| ETV6_v6 | 1.21 | 2.50 | 5 | ACCGGAAGTG | 132 | 7 |
| CREB3L1_v12 | 1.21 | 2.50 | 5 | ATGCCACGTCAGCA | 547 | 0 |
| DLX1_v24 | 1.21 | 2.50 | 6 | GTCATTAC | | 0 |
| TP53_v8 | 1.20 | 2.48 | 6 | AACATGCCCGGACATGT | 523 | 0 |
| | | | | C | | |
| SP3_v1 | 1.20 | 2.48 | 6 | GGCCACGCCCACC | 548 | 10 |
| ZNF281_v15 | 1.20 | 2.48 | 5 | GGGGGAAGGGAG | 500 | 3 |
| RREB1_v21 | 1.19 | 2.46 | 5 | CCCCAAAACAACCCCCC | 549 | 10 |
| | | | | CCC | | |
| MYCN_v3 | 1.19 | 2.45 | 5 | GGCCACGTGGCC | 550 | 3 |
| TWIST1_v22 | 1.18 | 2.44 | 5 | ATTGCAGATGTTT | 340 | 7 |
| NPAS2_v1 | 1.17 | 2.41 | 5 | GGCACGTGTC | 317 | 10 |

TABLE 1J
Core Promoter Sequences

| SEQ | | |
|---|---|---|
| ID | | |
| NO: | Name | Sequence |
| 558 | PR181 | CATACTGAAAAGCATACTTTTGCAATGTTATTTTTAAAAA |
| | | CAAGGAACTCTTTAACCCAGGGAAGATAATCACTTGGGGA |
| | | AAGGAAGGTTCGTTTCTGAGTTAGCAACAAGTAAATGCAG |
| | | CACTAGTGGGTGGGATTGAGGTATGCCCTGGTGCATAAAT |
| | | AGAGACTCAGCTGTGCTGGCACACTCAGAAGCTTGGACCG |
| | | CATCCTAGCCGCCGACTCACACAAGGCAGGTGGGTGAGGA |
| | | AATCCAGGTAAGGCTCCTGACAGCAGCTTTAGAAGGGTAC |
| | | TTGCTGGAGTGAATTCGGGCCTCTGATTA |
| 559 | PR180 | ACCTCTTAACAATACGTTTCACAAATAGTTAAAAACATGC |
| | | ATACTGAAAAGCATACTTTTGCAATGTTATTTTTAAAAAC |
| | | AAGGAACTCTTTAACCCAGGGAAGATAATCACTTGGGGAA |
| | | AGGAAGGTTCGTTTCTGAGTTAGCAACAAGTAAATGCAGC |
| | | ACTAGTGGGTGGGATTGAGGTgTGCCCTGGTGCATAAATA |
| | | GAGACTCAGCTGTGCTGGCACACTCAAGAAGCTTGGACCG |
| | | CATCCTAGCCGCCGACTCACACAAGGCAGGTGGGTGAGGA |
| | | AATCCAGGTAAGGCTCCTGACAGCAGCTTTAGAAGGGTAC |
| | | TTGCTGGAGTGAATTCGGGCCTCTGATT |
| 560 | PR179 | CCGGCCCGCCCCCTTTCCTTACGCGGATTGGTAGCTGCAG |
| | | GCTTCCCTATCTGATTGGCCGAACGAACGCAGCGCGTAAT |
| | | TTAAAATATTGTATCTGTAACAAAGCTGCACCTCGTGGGC |
| | | GGAGTTGTGCTCTGCGGCTGCGAAAGTCCAGCTTCGGCGA |
| | | CTAGGTGTGAGTAAGCCAcggcggcgcagatcgcccggcg |
| | | cggctccgccccctgcgccggtcacgtgggggcgccggct |
| | | gcgcctgcggagaagcggtggccgccgagcgggatctgtg |
| | | cggggagccggaaatggttgtggactacgtctgtgcggct |
| | | gcgtggggctcggccgcgcggactgaaggagactgaaggt |
| | | gctggggggaccctgatgtggA |
| 561 | PR178 | CCGGCCCGCCCCCTTTCCTTACGCGGATTGGTAGCTGCAG |
| | | GCTTCCCTATCTGATTGGCCGAACGAACGCAGCGCGTAAT |
| | | TTAAAATATTGTATCTGTAACAAAGCTGCACCTCGTGGGC |
| | | GGAGTTGTGCTCTGCGGCTGCGAAAGTCCAGCTTCGGCGA |
| | | CTAGGTGTGAGTAAGCCACtttttccgtgctacctgcaga |
| | | ggggtccatacggcgttgttctggattcACCGGTa |
| 562 | PR177 | CCGGCCCGCCCCCTTTCCTTACGCGGATTGGTAGCTGCAG |
| | | GCTTCCCTATCTGATTGGCCGAACGAACGCAGCGCGTAAT |
| | | TTAAAATATTGTATCTGTAACAAAGCTGCACCTCGTGGGC |
| | | GGAGTTGTGCTCTGCGGCTGCGAAAGTCCAGCTTCGGCGA |
| | | CTAGGTGTGAGTAAGCCACACTCGCGCTGCCATCACTCTT |
| | | CCGCCGTCTTCGCCGCCATCCTCGGCGCGACTCGCTTCTT |
| | | TCGGTTCTACCAGGTAGAGTCCGCCGCCATCCTCA |
| 563 | PR176 | CCGGCCCGCCCCCTTTCCTTACGCGGATTGGTAGCTGCAG |
| | | GCTTCCCTATCTGATTGGCCGAACGAACGCAGCGCGTAAT |
| | | TTAAAATATTGTATCTGTAACAAAGCTGCACCTCGTGGGC |
| | | GGAGTTGTGCTCTGCGGCTGCGAAAGTCCAGCTTCGGCGA |
| | | CTAGGTGTGAGTAAGCCAGAAGCTTGGACCGCATCCTAGC |
| | | CGCCGACTCACACAAGGCAGGTGGGTGAGGAAATCCAGGT |
| | | AAGGCTCCTGACAGCAGCTTTAGAAGGGTACTTGCTGGAG |
| | | TGAATTCGGGCCTCTGATTA |
| 564 | PR175 | CCGGCCCGCCCCCTTTCCTTACGCGGATTGGTAGCTGCAG |
| | | GCTTCCCTATCTGATTGGCCGAACGAACGCAGCGCGTAAT |
| | | TTAAAATATTGTATCTGTAACAAAGCTGCACCTCGTGGGC |
| | | GGAGTTGTGCTCTGCGGCTGCGAAAGTCCAGCTTCGGCGA |
| | | CTAGGTGTGAGTAAGCCAAAATCCAGAGCGGCGGGCACTG |
| | | ACGGGCACTTGCACCGTGTGGACAGACTCTCCGGTTCTGT |
| | | GAGTGGTTTTTCTTTTCCCGGGTCGGACCTGGAGTTCTTA |
| | | GGGGGATGGCTGAAgaattcA |
| 565 | PR174 | CACCTCTTAACAATACGTTTCACAAATAGTTAAAAACATG |
| | | CATACTGAAAAGCATACTTTTGCAATGTTATTTTTAAAAA |
| | | CAAGGAACTCTTTAACCCAGGGAAGATAATCACTTGGGGA |
| | | AAGGAAGGTTCGTTTCTGAGTTAGCAACAAGTAAATGCAG |
| | | CACTAGTGGGTGGGATTGAGGTgTGCCCTGGTGCATAAAT |
| | | AGAGACTCAGCTGTGCTGGCACACTCAAcggcggcgcaga |
| | | tcgcccggcgcggctccgccccctgcgccggtcacgtggg |
| | | ggcgccggctgcgcctgcggagaagcggtggccgccgagc |
| | | gggatctgtgcggggagccggaaatggttgtggactacgt |
| | | ctgtgcggctgcgtggggctcggccgcgcggactgaagga |
| | | gactgaaggtgctggggggaccctgatgtggA |
| 566 | PR173 | CACCTCTTAACAATACGTTTCACAAATAGTTAAAAACATG |
| | | CATACTGAAAAGCATACTTTTGCAATGTTATTTTTAAAAA |
| | | CAAGGAACTCTTTAACCCAGGGAAGATAATCACTTGGGGA |
| | | AAGGAAGGTTCGTTTCTGAGTTAGCAACAAGTAAATGCAG |
| | | CACTAGTGGGTGGGATTGAGGTgTGCCCTGGTGCATAAAT |
| | | AGAGACTCAGCTGTGCTGGCACACTCAACtttttccgtgc |
| | | tacctgcagaggggtccatacggcgttgttctggattca |
| 567 | PR172 | CACCTCTTAACAATACGTTTCACAAATAGTTAAAAACATG |
| | | CATACTGAAAAGCATACTTTTGCAATGTTATTTTTAAAAA |
| | | CAAGGAACTCTTTAACCCAGGGAAGATAATCACTTGGGGA |
| | | AAGGAAGGTTCGTTTCTGAGTTAGCAACAAGTAAATGCAG |
| | | CACTAGTGGGTGGGATTGAGGTgTGCCCTGGTGCATAAAT |
| | | AGAGACTCAGCTGTGCTGGCACACTCAACACTCGCGCTGC |
| | | CATCACTCTTCCGCCGTCTTCGCCGCCATCCTCGGCGCGA |
| | | CTCGCTTCTTTCGGTTCTACCAGGTAGAGTCCGCCGCCAT |
| | | CCTCA |
| 568 | PR171 | CACCTCTTAACAATACGTTTCACAAATAGTTAAAAACATG |
| | | CATACTGAAAAGCATACTTTTGCAATGTTATTTTTAAAAA |
| | | CAAGGAACTCTTTAACCCAGGGAAGATAATCACTTGGGGA |
| | | AAGGAAGGTTCGTTTCTGAGTTAGCAACAAGTAAATGCAG |
| | | CACTAGTGGGTGGGATTGAGGTgTGCCCTGGTGCATAAAT |
| | | AGAGACTCAGCTGTGCTGGCACACTCAAGTATCCCAGGAG |
| | | GAGCAAGTGGCACGTCTTCGGGTGAGTGTGCGGCTGTGCT |
| | | GGAGCCCGGGTTACCAGCTCTTAA |
| 569 | PR170 | CACCTCTTAACAATACGTTTCACAAATAGTTAAAAACATG |
| | | CATACTGAAAAGCATACTTTTGCAATGTTATTTTTAAAAA |
| | | CAAGGAACTCTTTAACCCAGGGAAGATAATCACTTGGGGA |
| | | AAGGAAGGTTCGTTTCTGAGTTAGCAACAAGTAAATGCAG |
| | | CACTAGTGGGTGGGATTGAGGTgTGCCCTGGTGCATAAAT |
| | | AGAGACTCAGCTGTGCTGGCACACTCAAAAATCCAGAGCG |
| | | GCGGGCACTGACGGGCACTTGCACCGTGTGGACAGACTCT |
| | | CCGGTTCTGTGAGTGGTTTTTCTTTTCCCGGGTCGGACCT |
| | | GGAGTTCTTAGGGGGATGGCTGAAgaattcA |
| 570 | PR169 | CGGGAAAAGTTCAGCTGAGAGATATAAAAGAGCAGTCTTT |
| | | CCAGCACCTGCcggcggcgcagatcgcccggcgcggctcc |
| | | gccccctgcgccggtcacgtgggggcgccggctgcgcctg |
| | | cggagaagcggtggccgccgagcgggatctgtgcggggag |
| | | ccggaaatggttgtggactacgtctgtgcggctgcgtggg |
| | | gctcggccgcgcggactgaaggagactgaaggtgctgggg |
| | | ggaccctgatgtggA |
| 571 | PR168 | CGGGAAAAGTTCAGCTGAGAGATATAAAAGAGCAGTCTTT |
| | | CCAGCACCTGCCtttttccgtgctacctgcagaggggtcc |
| | | atacggcgttgttctggattca |
| 572 | PR167 | CGGGAAAAGTTCAGCTGAGAGATATAAAAGAGCAGTCTTT |
| | | CCAGCACCTGCCACTCGCGCTGCCATCACTCTTCCGCCGT |
| | | CTTCGCCGCCATCCTCGGCGCGACTCGCTTCTTTCGGTTC |
| | | TACCAGGTAGAGTCCGCCGCCATCCTCA |
| 573 | PR166 | CGGGAAAAGTTCAGCTGAGAGATATAAAAGAGCAGTCTTT |
| | | CCAGCACCTGCGTATCCCAGGAGGAGCAAGTGGCACGTCT |
| | | TCGGGTGAGTGTGCGGCTGTGCTGGAGCCCGGGTTACCAG |
| | | CTCTTAA |
| 574 | PR165 | CGGGAAAAGTTCAGCTGAGAGATATAAAAGAGCAGTCTTT |
| | | CCAGCACCTGCGAAGCTTGGACCGCATCCTAGCCGCCGAC |
| | | TCACACAAGGCAGGTGGGTGAGGAAATCCAGGTAAGGCTC |
| | | CTGACAGCAGCTTTAGAAGGGTACTTGCTGGAGTGAATTC |
| | | GGGCCTCTGATTA |
| 575 | PR159 | agcttgcatgcctgcaggtcggagtactgtcctccgagcg |
| | | gagtactgtcctccgagcggagtactgtcctccgagcgga |
| | | gtactgtcctccgagcggagtactgtcctccgagcggtgc |
| | | gctcccgacatgccccgcggcgcgccattaaccgccagat |
| | | ttgagtcgcgggacccgttggcagaggtggg |
| 576 | PR156 | AGTGGTGGGGGAGTGAAAAGAGAGATGGAGAAAGAGGGGA |
| | | TGGGCAGAAAGAGGAGGAGGAGTCAGGGGCAGGGCATGGA |
| | | GGTGGGTGGGGCTGGGCTGCCAAAGCAGGATAAATGCACA |
| | | CCTGCCTGCTGGTCTGGGCTCCCTGCCTCGGGCTCTCACC |
| | | CTCCTCTCCTGCAGCTCCAGCTTTGTGCTCT |
| 577 | PR155 | CATACTGAAAAGCATACTTTTGCAATGTTATTTTTAAAAA |
| | | CAAGGAACTCTTTAACCCAGGGAAGATAATCACTTGGGGA |
| | | AAGGAAGGTTCGTTTCTGAGTTAGCAACAAGTAAATGCAG |
| | | CACTAGTGGGTGGGATTGAGGTGTGCCCTGGTGCATAAAT |
| | | AGAGACTCAGCTGTGCTGGCACACTCAGAAGCTTGGACCG |
| | | CATCCTAGCCGCCGACTCACACAAGGCAGGTGGGTGAGGA |
| | | AATCCAGGTAAGGCTCCTGACAGCAGCTTTAGAAGGGTAC |
| | | TTGCTGGAGTG |
| 578 | PR154 | GGCCCGCCCCCTTTCCTTACGCGGATTGGTAGCTGCAGGC |
| | | TTCCCTATCTGATTGGCCGAACGAACGCAGCGCGTAATTT |
| | | AAAATATTGTATCTGTAACAAAGCTGCACCTCGTGGGCGG |
| | | AGTTGTGCTCTGCGGCTGCGAAAGTCCAGCTTCGGCGACT |
| | | AGGTGTGAGTAAGCCAGTATCCCAGGAGGAGCAAGTGGCA |
| | | CGTCTTCGGGTGAGTGTGCGGCTGTGCTGGAGCCCGGGTT |
| | | ACCAGCTCTT |
| 579 | PR153 | GGGAAAAGTTCAGCTGAGAGATATAAAAGAGCAGTCTTTC |
| | | CAGCACCTGCAAATCCAGAGCGGCGGGCACTGACGGGCAC |
| | | TTGCACCGTGTGGACAGACTCTCCGGTTCTGTGAGTGGTT |
| | | TTTCTTTTCCCGGGTCGGACCTGGAGTTCTTAGGGGGATG |
| | | GCTGa |
| 580 | PR152 | ACCCACGTGATGCTGAGAAGTACTCCTGCCCTAGGAAGAG |
| | | ACTCAGGGCAGAGGGAGGAAGGACAGCAGACCAGACAGTC |
| | | ACAGCAGCCTTGACAAAACGTTCCTGGAAC |
| 581 | PR151 | TATAAAAGGCCAGCAGCAGCCTGACCACATCTCATCC |
| 582 | PR150 | CACTCCCAGAAGGCAGCGGGCGAGGGCGTGGGGCCGGGGC |
| | | TCTCCCGGCATGCTCTGCGGCGCGCCTCCGCCCGCGCGAT |
| | | TTGAATCCTGCGTTTGAGTCGTCTTGGCGGAGGTTGTGGT |
| | | GACGC |
| 583 | PR131 | tcccgacatgccccgcggcgcgccattaaccgccagattt |
| | | gagtcgcgggacccgttggcagaggtg |
| 584 | | GTATCCCAGGAGGAGCAAGTGGCACGTCTTCGGGT |
| 585 | | CGGGAAAAGTTCAGCTGAGAGATATAAAAGAGCAGTCTTT |
| | | CCAGCACCTGC |
| 586 | | GTATCCCAGGAGGAGCAAGTGGCACGTCTTCGGGTGAGTG |
| | | TGCGGCTGTGCTGGAGCCCGGGTTACCAGCTCTTAA |
| 587 | | CAGTGTGCGGCTGTGCTGGAGCCCGGGTTACCAGCTCTT |

In some embodiments, the sequence of any of the core promoters listed in Table 1J can further comprise, at the 5′ end, any of SEQ ID NOs: 377-397 listed in Table 1B, or reverse complements thereof. In some embodiments, the sequence of any of the core promoters listed in Table 1J can further comprise, at the 5′ end, any of SEQ ID NOs: 377-397 listed in Table 1B, or reverse complements thereof, in a vector. In some embodiments, the sequence of any of the core promoters listed in Table 1J can further comprise, at the 5′ end, any of SEQ ID NOs: 377-397 listed in Table 1B, or reverse complements thereof, in a nanoplasmid. In some embodiments, the sequence of any of the core promoters listed in Table 1J can further comprise, at the 5′ end, any of SEQ ID NOs: 377-397 listed in Table 1B, or reverse complements thereof, in a linked double-stranded DNA.

In an embodiment, PR181 can further comprise, at the 5′ end, a sequence comprising SRE010, SRE012, SRE007, and SRE008, or a reverse complement thereof, in a vector. In some embodiments, PR180 can further comprise, at the 5′ end, a sequence comprising SRE010, SRE012, SRE007, and SRE008, or a reverse complement thereof, in a vector. In some embodiments, PR179 can further comprise, at the 5′ end, a sequence comprising SRE010, SRE012, SRE007, and SRE008, or a reverse complement thereof, in a vector. In some embodiments, PR178 can further comprise, at the 5′ end, a sequence comprising SRE010, SRE012, SRE007, and SRE008, or a reverse complement thereof, in a vector. In some embodiments, PR177 can further comprise, at the 5′ end, a sequence comprising SRE010, SRE012, SRE007, and SRE008, or a reverse complement thereof, in a vector. In some embodiments, PR176 can further comprise, at the 5′ end, a sequence comprising SRE010, SRE012, SRE007, and SRE008, or a reverse complement thereof, in a vector. In some embodiments, PR175 can further comprise, at the 5′ end, a sequence comprising SRE010, SRE012, SRE007, and SRE008, or a reverse complement thereof, in a vector. In some embodiments, PR174 can further comprise, at the 5′ end, a sequence comprising SRE010, SRE012, SRE007, and SRE008, or a reverse complement thereof, in a vector. In some embodiments, PR173 can further comprise, at the 5′ end, a sequence comprising SRE010, SRE012, SRE007, and SRE008, or a reverse complement thereof, in a vector. In some embodiments, PR172 can further comprise, at the 5′ end, a sequence comprising SRE010, SRE012, SRE007, and SRE008, or a reverse complement thereof, in a vector. In some embodiments, PR171 can further comprise, at the 5′ end, a sequence comprising SRE010, SRE012, SRE007, and SRE008, or a reverse complement thereof, in a vector. In some embodiments, PR170 can further comprise, at the 5′ end, a sequence comprising SRE010, SRE012, SRE007, and SRE008, or a reverse complement thereof, in a vector. In some embodiments, PR169 can further comprise, at the 5′ end, a sequence comprising SRE010, SRE012, SRE007, and SRE008, or a reverse complement thereof, in a vector. In some embodiments, PR168 can further comprise, at the 5′ end, a sequence comprising SRE010, SRE012, SRE007, and SRE008, or a reverse complement thereof, in a vector. In some embodiments, PR167 can further comprise, at the 5′ end, a sequence comprising SRE010, SRE012, SRE007, and SRE008, or a reverse complement thereof, in a vector. In some embodiments, PR166 can further comprise, at the 5′ end, a sequence comprising SRE010, SRE012, SRE007, and SRE008, or a reverse complement thereof, in a vector. In some embodiments, PR165 can further comprise, at the 5′ end, a sequence comprising SRE010, SRE012, SRE007, and SRE008, or a reverse complement thereof, in a vector. In some embodiments, PR159 can further comprise, at the 5′ end, a sequence comprising SRE010, SRE012, SRE007, and SRE008, or a reverse complement thereof, in a vector. In some embodiments, PR156 can further comprise, at the 5′ end, a sequence comprising SRE010, SRE012, SRE007, and SRE008, or a reverse complement thereof, in a vector. In some embodiments, PR155 can further comprise, at the 5′ end, a sequence comprising SRE010, SRE012, SRE007, and SRE008, or a reverse complement thereof, in a vector. In some embodiments, PR154 can further comprise, at the 5′ end, a sequence comprising SRE010, SRE012, SRE007, and SRE008, or a reverse complement thereof, in a vector. In some embodiments, PR153 can further comprise, at the 5′ end, a sequence comprising SRE010, SRE012, SRE007, and SRE008, or a reverse complement thereof, in a vector. In some embodiments, PR152 can further comprise, at the 5′ end, a sequence comprising SRE010, SRE012, SRE007, and SRE008, or a reverse complement thereof, in a vector. In some embodiments, PR151 can further comprise, at the 5′ end, a sequence comprising SRE010, SRE012, SRE007, and SRE008, or a reverse complement thereof, in a vector. In some embodiments, PR150 can further comprise, at the 5′ end, a sequence comprising SRE010, SRE012, SRE007, and SRE008, or a reverse complement thereof, in a vector. In some embodiments, PR131 can further comprise, at the 5′ end, a sequence comprising SRE010, SRE012, SRE007, and SRE008, or a reverse complement thereof, in a vector. In some embodiments, SEQ ID NO: 584 can further comprise, at the 5′ end, a sequence comprising SRE010, SRE012, SRE007, and SRE008, or a reverse complement thereof, in a vector. In some embodiments, SEQ ID NO: 585 can further comprise, at the 5′ end, a sequence comprising SRE010, SRE012, SRE007, and SRE008, or a reverse complement thereof, in a vector. In some embodiments, SEQ ID NO: 586 can further comprise, at the 5′ end, a sequence comprising SRE010, SRE012, SRE007, and SRE008, or a reverse complement thereof, in a vector. In some embodiments, SEQ ID NO: 587 can further comprise, at the 5′ end, a sequence comprising SRE010, SRE012, SRE007, and SRE008, or a reverse complement thereof, in a vector. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In an embodiment, PR181 can further comprise, at the 5′ end, a sequence comprising SRE010, SRE012, SRE007, and SRE008, or a reverse complement thereof, in a nanoplasmid. In some embodiments, PR180 can further comprise, at the 5′ end, a sequence comprising SRE010, SRE012, SRE007, and SRE008, or a reverse complement thereof, in a nanoplasmid. In some embodiments, PR179 can further comprise, at the 5′ end, a sequence comprising SRE010, SRE012, SRE007, and SRE008, or a reverse complement thereof, in a nanoplasmid. In some embodiments, PR178 can further comprise, at the 5′ end, a sequence comprising SRE010, SRE012, SRE007, and SRE008, or a reverse complement thereof, in a nanoplasmid. In some embodiments, PR177 can further comprise, at the 5′ end, a sequence comprising SRE010, SRE012, SRE007, and SRE008, or a reverse complement thereof, in a nanoplasmid. In some embodiments, PR176 can further comprise, at the 5′ end, a sequence comprising SRE010, SRE012, SRE007, and SRE008, or a reverse complement thereof, in a nanoplasmid. In some embodiments, PR175 can further comprise, at the 5′ end, a sequence comprising SRE010, SRE012, SRE007, and SRE008, or a reverse complement thereof, in a nanoplasmid. In some embodiments, PR174 can further comprise, at the 5′ end, a sequence comprising SRE010, SRE012, SRE007, and SRE008, or a reverse complement thereof, in a nanoplasmid. In some embodiments, PR173 can further comprise, at the 5′ end, a sequence comprising SRE010, SRE012, SRE007, and SRE008, or a reverse complement thereof, in a nanoplasmid. In some embodiments, PR172 can further comprise, at the 5′ end, a sequence comprising SRE010, SRE012, SRE007, and SRE008, or a reverse complement thereof, in a nanoplasmid. In some embodiments, PR171 can further comprise, at the 5′ end, a sequence comprising SRE010, SRE012, SRE007, and SRE008, or a reverse complement thereof, in a nanoplasmid. In some embodiments, PR170 can further comprise, at the 5′ end, a sequence comprising SRE010, SRE012, SRE007, and SRE008, or a reverse complement thereof, in a nanoplasmid. In some embodiments, PR169 can further comprise, at the 5′ end, a sequence comprising SRE010, SRE012, SRE007, and SRE008, or a reverse complement thereof, in a nanoplasmid. In some embodiments, PR168 can further comprise, at the 5′ end, a sequence comprising SRE010, SRE012, SRE007, and SRE008, or a reverse complement thereof, in a nanoplasmid. In some embodiments, PR167 can further comprise, at the 5′ end, a sequence comprising SRE010, SRE012, SRE007, and SRE008, or a reverse complement thereof, in a nanoplasmid. In some embodiments, PR166 can further comprise, at the 5′ end, a sequence comprising SRE010, SRE012, SRE007, and SRE008, or a reverse complement thereof, in a nanoplasmid. In some embodiments, PR165 can further comprise, at the 5′ end, a sequence comprising SRE010, SRE012, SRE007, and SRE008, or a reverse complement thereof, in a nanoplasmid. In some embodiments, PR159 can further comprise, at the 5′ end, a sequence comprising SRE010, SRE012, SRE007, and SRE008, or a reverse complement thereof, in a nanoplasmid. In some embodiments, PR156 can further comprise, at the 5′ end, a sequence comprising SRE010, SRE012, SRE007, and SRE008, or a reverse complement thereof, in a nanoplasmid. In some embodiments, PR155 can further comprise, at the 5′ end, a sequence comprising SRE010, SRE012, SRE007, and SRE008, or a reverse complement thereof, in a nanoplasmid. In some embodiments, PR154 can further comprise, at the 5′ end, a sequence comprising SRE010, SRE012, SRE007, and SRE008, or a reverse complement thereof, in a nanoplasmid. In some embodiments, PR153 can further comprise, at the 5′ end, a sequence comprising SRE010, SRE012, SRE007, and SRE008, or a reverse complement thereof, in a nanoplasmid. In some embodiments, PR152 can further comprise, at the 5′ end, a sequence comprising SRE010, SRE012, SRE007, and SRE008, or a reverse complement thereof, in a nanoplasmid. In some embodiments, PR151 can further comprise, at the 5′ end, a sequence comprising SRE010, SRE012, SRE007, and SRE008, or a reverse complement thereof, in a nanoplasmid. In some embodiments, PR150 can further comprise, at the 5′ end, a sequence comprising SRE010, SRE012, SRE007, and SRE008, or a reverse complement thereof, in a nanoplasmid. In some embodiments, PR131 can further comprise, at the 5′ end, a sequence comprising SRE010, SRE012, SRE007, and SRE008, or a reverse complement thereof, in a nanoplasmid. In some embodiments, SEQ ID NO: 584 can further comprise, at the 5′ end, a sequence comprising SRE010, SRE012, SRE007, and SRE008, or a reverse complement thereof, in a nanoplasmid. In some embodiments, SEQ ID NO: 585 can further comprise, at the 5′ end, a sequence comprising SRE010, SRE012, SRE007, and SRE008, or a reverse complement thereof, in a nanoplasmid. In some embodiments, SEQ ID NO: 586 can further comprise, at the 5′ end, a sequence comprising SRE010, SRE012, SRE007, and SRE008, or a reverse complement thereof, in a nanoplasmid. In some embodiments, SEQ ID NO: 587 can further comprise, at the 5′ end, a sequence comprising SRE010, SRE012, SRE007, and SRE008, or a reverse complement thereof, in a nanoplasmid. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In an embodiment, PR181 can further comprise, at the 5′ end, a sequence comprising SRE010, SRE012, SRE007, and SRE008, or a reverse complement thereof, in a linked double-stranded DNA. In some embodiments, PR180 can further comprise, at the 5′ end, a sequence comprising SRE010, SRE012, SRE007, and SRE008, optionally in a vector, further optionally, in a nanoplasmid or linked double-stranded DNA. In some embodiments, PR179 can further comprise, at the 5′ end, a sequence comprising SRE010, SRE012, SRE007, and SRE008, or a reverse complement thereof, in a linked double-stranded DNA. In some embodiments, PR178 can further comprise, at the 5′ end, a sequence comprising SRE010, SRE012, SRE007, and SRE008, or a reverse complement thereof, in a linked double-stranded DNA. In some embodiments, PR177 can further comprise, at the 5′ end, a sequence comprising SRE010, SRE012, SRE007, and SRE008, or a reverse complement thereof, in a linked double-stranded DNA. In some embodiments, PR176 can further comprise, at the 5′ end, a sequence comprising SRE010, SRE012, SRE007, and SRE008, or a reverse complement thereof, in a linked double-stranded DNA. In some embodiments, PR175 can further comprise, at the 5′ end, a sequence comprising SRE010, SRE012, SRE007, and SRE008, or a reverse complement thereof, in a linked double-stranded DNA. In some embodiments, PR174 can further comprise, at the 5′ end, a sequence comprising SRE010, SRE012, SRE007, and SRE008, or a reverse complement thereof, in a linked double-stranded DNA. In some embodiments, PR173 can further comprise, at the 5′ end, a sequence comprising SRE010, SRE012, SRE007, and SRE008, or a reverse complement thereof, in a linked double-stranded DNA. In some embodiments, PR172 can further comprise, at the 5′ end, a sequence comprising SRE010, SRE012, SRE007, and SRE008, or a reverse complement thereof, in a linked double-stranded DNA. In some embodiments, PR171 can further comprise, at the 5′ end, a sequence comprising SRE010, SRE012, SRE007, and SRE008, or a reverse complement thereof, in a linked double-stranded DNA. In some embodiments, PR170 can further comprise, at the 5′ end, a sequence comprising SRE010, SRE012, SRE007, and SRE008, or a reverse complement thereof, in a linked double-stranded DNA. In some embodiments, PR169 can further comprise, at the 5′ end, a sequence comprising SRE010, SRE012, SRE007, and SRE008, or a reverse complement thereof, in a linked double-stranded DNA. In some embodiments, PR168 can further comprise, at the 5′ end, a sequence comprising SRE010, SRE012, SRE007, and SRE008, or a reverse complement thereof, in a linked double-stranded DNA. In some embodiments, PR167 can further comprise, at the 5′ end, a sequence comprising SRE010, SRE012, SRE007, and SRE008, or a reverse complement thereof, in a linked double-stranded DNA. In some embodiments, PR166 can further comprise, at the 5′ end, a sequence comprising SRE010, SRE012, SRE007, and SRE008, or a reverse complement thereof, in a linked double-stranded DNA. In some embodiments, PR165 can further comprise, at the 5′ end, a sequence comprising SRE010, SRE012, SRE007, and SRE008, or a reverse complement thereof, in a linked double-stranded DNA. In some embodiments, PR159 can further comprise, at the 5′ end, a sequence comprising SRE010, SRE012, SRE007, and SRE008, or a reverse complement thereof, in a linked double-stranded DNA. In some embodiments, PR156 can further comprise, at the 5′ end, a sequence comprising SRE010, SRE012, SRE007, and SRE008, or a reverse complement thereof, in a linked double-stranded DNA. In some embodiments, PR155 can further comprise, at the 5′ end, a sequence comprising SRE010, SRE012, SRE007, and SRE008, or a reverse complement thereof, in a linked double-stranded DNA. In some embodiments, PR154 can further comprise, at the 5′ end, a sequence comprising SRE010, SRE012, SRE007, and SRE008, or a reverse complement thereof, in a linked double-stranded DNA. In some embodiments, PR153 can further comprise, at the 5′ end, a sequence comprising SRE010, SRE012, SRE007, and SRE008, or a reverse complement thereof, in a linked double-stranded DNA. In some embodiments, PR152 can further comprise, at the 5′ end, a sequence comprising SRE010, SRE012, SRE007, and SRE008, or a reverse complement thereof, in a linked double-stranded DNA. In some embodiments, PR151 can further comprise, at the 5′ end, a sequence comprising SRE010, SRE012, SRE007, and SRE008, or a reverse complement thereof, in a linked double-stranded DNA. In some embodiments, PR150 can further comprise, at the 5′ end, a sequence comprising SRE010, SRE012, SRE007, and SRE008, or a reverse complement thereof, in a linked double-stranded DNA. In some embodiments, PR131 can further comprise, at the 5′ end, a sequence comprising SRE010, SRE012, SRE007, and SRE008, or a reverse complement thereof, in a linked double-stranded DNA. In some embodiments, SEQ ID NO: 584 can further comprise, at the 5′ end, a sequence comprising SRE010, SRE012, SRE007, and SRE008, or a reverse complement thereof, in a linked double-stranded DNA. In some embodiments, SEQ ID NO: 585 can further comprise, at the 5′ end, a sequence comprising SRE010, SRE012, SRE007, and SRE008, or a reverse complement thereof, in a linked double-stranded DNA. In some embodiments, SEQ ID NO: 586 can further comprise, at the 5′ end, a sequence comprising SRE010, SRE012, SRE007, and SRE008, or a reverse complement thereof, in a linked double-stranded DNA. In some embodiments, SEQ ID NO: 587 can further comprise, at the 5′ end, a sequence comprising SRE010, SRE012, SRE007, and SRE008, or a reverse complement thereof, in a linked double-stranded DNA. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In an embodiment, PR181 can further comprise, at the 5′ end, a sequence comprising SRE010, SRE007, and SRE008, or a reverse complement thereof, in a vector. In some embodiments, PR180 can further comprise, at the 5′ end, a sequence comprising SRE010, SRE007, and SRE008, or a reverse complement thereof, in a vector. In some embodiments, PR179 can further comprise, at the 5′ end, a sequence comprising SRE010, SRE007, and SRE008, or a reverse complement thereof, in a vector. In some embodiments, PR178 can further comprise, at the 5′ end, a sequence comprising SRE010, SRE007, and SRE008, or a reverse complement thereof, in a vector. In some embodiments, PR177 can further comprise, at the 5′ end, a sequence comprising SRE010, SRE007, and SRE008, or a reverse complement thereof, in a vector. In some embodiments, PR176 can further comprise, at the 5′ end, a sequence comprising SRE010, SRE007, and SRE008, or a reverse complement thereof, in a vector. In some embodiments, PR175 can further comprise, at the 5′ end, a sequence comprising SRE010, SRE007, and SRE008, or a reverse complement thereof, in a vector. In some embodiments, PR174 can further comprise, at the 5′ end, a sequence comprising SRE010, SRE007, and SRE008, or a reverse complement thereof, in a vector. In some embodiments, PR173 can further comprise, at the 5′ end, a sequence comprising SRE010, SRE007, and SRE008, or a reverse complement thereof, in a vector. In some embodiments, PR172 can further comprise, at the 5′ end, a sequence comprising SRE010, SRE007, and SRE008, or a reverse complement thereof, in a vector. In some embodiments, PR171 can further comprise, at the 5′ end, a sequence comprising SRE010, SRE007, and SRE008, or a reverse complement thereof, in a vector. In some embodiments, PR170 can further comprise, at the 5′ end, a sequence comprising SRE010, SRE007, and SRE008, or a reverse complement thereof, in a vector. In some embodiments, PR169 can further comprise, at the 5′ end, a sequence comprising SRE010, SRE007, and SRE008, or a reverse complement thereof, in a vector. In some embodiments, PR168 can further comprise, at the 5′ end, a sequence comprising SRE010, SRE007, and SRE008, or a reverse complement thereof, in a vector. In some embodiments, PR167 can further comprise, at the 5′ end, a sequence comprising SRE010, SRE007, and SRE008, or a reverse complement thereof, in a vector. In some embodiments, PR166 can further comprise, at the 5′ end, a sequence comprising SRE010, SRE007, and SRE008, or a reverse complement thereof, in a vector. In some embodiments, PR165 can further comprise, at the 5′ end, a sequence comprising SRE010, SRE007, and SRE008, or a reverse complement thereof, in a vector. In some embodiments, PR159 can further comprise, at the 5′ end, a sequence comprising SRE010, SRE007, and SRE008, or a reverse complement thereof, in a vector. In some embodiments, PR156 can further comprise, at the 5′ end, a sequence comprising SRE010, SRE007, and SRE008, or a reverse complement thereof, in a vector. In some embodiments, PR155 can further comprise, at the 5′ end, a sequence comprising SRE010, SRE007, and SRE008, or a reverse complement thereof, in a vector. In some embodiments, PR154 can further comprise, at the 5′ end, a sequence comprising SRE010, SRE007, and SRE008, or a reverse complement thereof, in a vector. In some embodiments, PR153 can further comprise, at the 5′ end, a sequence comprising SRE010, SRE007, and SRE008, or a reverse complement thereof, in a vector. In some embodiments, PR152 can further comprise, at the 5′ end, a sequence comprising SRE010, SRE007, and SRE008, or a reverse complement thereof, in a vector. In some embodiments, PR151 can further comprise, at the 5′ end, a sequence comprising SRE010, SRE007, and SRE008, or a reverse complement thereof, in a vector. In some embodiments, PR150 can further comprise, at the 5′ end, a sequence comprising SRE010, SRE007, and SRE008, or a reverse complement thereof, in a vector. In some embodiments, PR131 can further comprise, at the 5′ end, a sequence comprising SRE010, SRE007, and SRE008, or a reverse complement thereof, in a vector. In some embodiments, SEQ ID NO: 584 can further comprise, at the 5′ end, a sequence comprising SRE010, SRE007, and SRE008, or a reverse complement thereof, in a vector. In some embodiments, SEQ ID NO: 585 can further comprise, at the 5′ end, a sequence comprising SRE010, SRE007, and SRE008, or a reverse complement thereof, in a vector. In some embodiments, SEQ ID NO: 586 can further comprise, at the 5′ end, a sequence comprising SRE010, SRE007, and SRE008, or a reverse complement thereof, in a vector. In some embodiments, SEQ ID NO: 587 can further comprise, at the 5′ end, a sequence comprising SRE010, SRE007, and SRE008, or a reverse complement thereof, in a vector. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In an embodiment, PR181 can further comprise, at the 5′ end, a sequence comprising SRE010, SRE007, and SRE008, or a reverse complement thereof, in a nanoplasmid. In some embodiments, PR180 can further comprise, at the 5′ end, a sequence comprising SRE010, SRE007, and SRE008, or a reverse complement thereof, in a nanoplasmid. In some embodiments, PR179 can further comprise, at the 5′ end, a sequence comprising SRE010, SRE007, and SRE008, or a reverse complement thereof, in a nanoplasmid. In some embodiments, PR178 can further comprise, at the 5′ end, a sequence comprising SRE010, SRE007, and SRE008, or a reverse complement thereof, in a nanoplasmid. In some embodiments, PR177 can further comprise, at the 5′ end, a sequence comprising SRE010, SRE007, and SRE008, or a reverse complement thereof, in a nanoplasmid. In some embodiments, PR176 can further comprise, at the 5′ end, a sequence comprising SRE010, SRE007, and SRE008, or a reverse complement thereof, in a nanoplasmid. In some embodiments, PR175 can further comprise, at the 5′ end, a sequence comprising SRE010, SRE007, and SRE008, or a reverse complement thereof, in a nanoplasmid. In some embodiments, PR174 can further comprise, at the 5′ end, a sequence comprising SRE010, SRE007, and SRE008, or a reverse complement thereof, in a nanoplasmid. In some embodiments, PR173 can further comprise, at the 5′ end, a sequence comprising SRE010, SRE007, and SRE008, or a reverse complement thereof, in a nanoplasmid. In some embodiments, PR172 can further comprise, at the 5′ end, a sequence comprising SRE010, SRE007, and SRE008, or a reverse complement thereof, in a nanoplasmid. In some embodiments, PR171 can further comprise, at the 5′ end, a sequence comprising SRE010, SRE007, and SRE008, or a reverse complement thereof, in a nanoplasmid. In some embodiments, PR170 can further comprise, at the 5′ end, a sequence comprising SRE010, SRE007, and SRE008, or a reverse complement thereof, in a nanoplasmid. In some embodiments, PR169 can further comprise, at the 5′ end, a sequence comprising SRE010, SRE007, and SRE008, or a reverse complement thereof, in a nanoplasmid. In some embodiments, PR168 can further comprise, at the 5′ end, a sequence comprising SRE010, SRE007, and SRE008, or a reverse complement thereof, in a nanoplasmid. In some embodiments, PR167 can further comprise, at the 5′ end, a sequence comprising SRE010, SRE007, and SRE008, or a reverse complement thereof, in a nanoplasmid. In some embodiments, PR166 can further comprise, at the 5′ end, a sequence comprising SRE010, SRE007, and SRE008, or a reverse complement thereof, in a nanoplasmid. In some embodiments, PR165 can further comprise, at the 5′ end, a sequence comprising SRE010, SRE007, and SRE008, or a reverse complement thereof, in a nanoplasmid. In some embodiments, PR159 can further comprise, at the 5′ end, a sequence comprising SRE010, SRE007, and SRE008, or a reverse complement thereof, in a nanoplasmid. In some embodiments, PR156 can further comprise, at the 5′ end, a sequence comprising SRE010, SRE007, and SRE008, or a reverse complement thereof, in a nanoplasmid. In some embodiments, PR155 can further comprise, at the 5′ end, a sequence comprising SRE010, SRE007, and SRE008, or a reverse complement thereof, in a nanoplasmid. In some embodiments, PR154 can further comprise, at the 5′ end, a sequence comprising SRE010, SRE007, and SRE008, or a reverse complement thereof, in a nanoplasmid. In some embodiments, PR153 can further comprise, at the 5′ end, a sequence comprising SRE010, SRE007, and SRE008, or a reverse complement thereof, in a nanoplasmid. In some embodiments, PR152 can further comprise, at the 5′ end, a sequence comprising SRE010, SRE007, and SRE008, or a reverse complement thereof, in a nanoplasmid. In some embodiments, PR151 can further comprise, at the 5′ end, a sequence comprising SRE010, SRE007, and SRE008, or a reverse complement thereof, in a nanoplasmid. In some embodiments, PR150 can further comprise, at the 5′ end, a sequence comprising SRE010, SRE007, and SRE008, or a reverse complement thereof, in a nanoplasmid. In some embodiments, PR131 can further comprise, at the 5′ end, a sequence comprising SRE010, SRE007, and SRE008, or a reverse complement thereof, in a nanoplasmid. In some embodiments, SEQ ID NO: 584 can further comprise, at the 5′ end, a sequence comprising SRE010, SRE007, and SRE008, or a reverse complement thereof, in a nanoplasmid. In some embodiments, SEQ ID NO: 585 can further comprise, at the 5′ end, a sequence comprising SRE010, SRE007, and SRE008, or a reverse complement thereof, in a nanoplasmid. In some embodiments, SEQ ID NO: 586 can further comprise, at the 5′ end, a sequence comprising SRE010, SRE007, and SRE008, or a reverse complement thereof, in a nanoplasmid. In some embodiments, SEQ ID NO: 587 can further comprise, at the 5′ end, a sequence comprising SRE010, SRE007, and SRE008, or a reverse complement thereof, in a nanoplasmid. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In an embodiment, PR181 can further comprise, at the 5′ end, a sequence comprising SRE010, SRE007, and SRE008, or a reverse complement thereof, in a linked double-stranded DNA. In some embodiments, PR180 can further comprise, at the 5′ end, a sequence comprising SRE010, SRE007, and SRE008, or a reverse complement thereof, in a linked double-stranded DNA. In some embodiments, PR179 can further comprise, at the 5′ end, a sequence comprising SRE010, SRE007, and SRE008, or a reverse complement thereof, in a linked double-stranded DNA. In some embodiments, PR178 can further comprise, at the 5′ end, a sequence comprising SRE010, SRE007, and SRE008, or a reverse complement thereof, in a linked double-stranded DNA. In some embodiments, PR177 can further comprise, at the 5′ end, a sequence comprising SRE010, SRE007, and SRE008, or a reverse complement thereof, in a linked double-stranded DNA. In some embodiments, PR176 can further comprise, at the 5′ end, a sequence comprising SRE010, SRE007, and SRE008, or a reverse complement thereof, in a linked double-stranded DNA. In some embodiments, PR175 can further comprise, at the 5′ end, a sequence comprising SRE010, SRE007, and SRE008, or a reverse complement thereof, in a linked double-stranded DNA. In some embodiments, PR174 can further comprise, at the 5′ end, a sequence comprising SRE010, SRE007, and SRE008, or a reverse complement thereof, in a linked double-stranded DNA. In some embodiments, PR173 can further comprise, at the 5′ end, a sequence comprising SRE010, SRE007, and SRE008, or a reverse complement thereof, in a linked double-stranded DNA. In some embodiments, PR172 can further comprise, at the 5′ end, a sequence comprising SRE010, SRE007, and SRE008, or a reverse complement thereof, in a linked double-stranded DNA. In some embodiments, PR171 can further comprise, at the 5′ end, a sequence comprising SRE010, SRE007, and SRE008, or a reverse complement thereof, in a linked double-stranded DNA. In some embodiments, PR170 can further comprise, at the 5′ end, a sequence comprising SRE010, SRE007, and SRE008, or a reverse complement thereof, in a linked double-stranded DNA. In some embodiments, PR169 can further comprise, at the 5′ end, a sequence comprising SRE010, SRE007, and SRE008, or a reverse complement thereof, in a linked double-stranded DNA. In some embodiments, PR168 can further comprise, at the 5′ end, a sequence comprising SRE010, SRE007, and SRE008, or a reverse complement thereof, in a linked double-stranded DNA. In some embodiments, PR167 can further comprise, at the 5′ end, a sequence comprising SRE010, SRE007, and SRE008, or a reverse complement thereof, in a linked double-stranded DNA. In some embodiments, PR166 can further comprise, at the 5′ end, a sequence comprising SRE010, SRE007, and SRE008, or a reverse complement thereof, in a linked double-stranded DNA. In some embodiments, PR165 can further comprise, at the 5′ end, a sequence comprising SRE010, SRE007, and SRE008, or a reverse complement thereof, in a linked double-stranded DNA. In some embodiments, PR159 can further comprise, at the 5′ end, a sequence comprising SRE010, SRE007, and SRE008, or a reverse complement thereof, in a linked double-stranded DNA. In some embodiments, PR156 can further comprise, at the 5′ end, a sequence comprising SRE010, SRE007, and SRE008, or a reverse complement thereof, in a linked double-stranded DNA. In some embodiments, PR155 can further comprise, at the 5′ end, a sequence comprising SRE010, SRE007, and SRE008, or a reverse complement thereof, in a linked double-stranded DNA. In some embodiments, PR154 can further comprise, at the 5′ end, a sequence comprising SRE010, SRE007, and SRE008, or a reverse complement thereof, in a linked double-stranded DNA. In some embodiments, PR153 can further comprise, at the 5′ end, a sequence comprising SRE010, SRE007, and SRE008, or a reverse complement thereof, in a linked double-stranded DNA. In some embodiments, PR152 can further comprise, at the 5′ end, a sequence comprising SRE010, SRE007, and SRE008, or a reverse complement thereof, in a linked double-stranded DNA. In some embodiments, PR151 can further comprise, at the 5′ end, a sequence comprising SRE010, SRE007, and SRE008, or a reverse complement thereof, in a linked double-stranded DNA. In some embodiments, PR150 can further comprise, at the 5′ end, a sequence comprising SRE010, SRE007, and SRE008, or a reverse complement thereof, in a linked double-stranded DNA. In some embodiments, PR131 can further comprise, at the 5′ end, a sequence comprising SRE010, SRE007, and SRE008, or a reverse complement thereof, in a linked double-stranded DNA. In some embodiments, SEQ ID NO: 584 can further comprise, at the 5′ end, a sequence comprising SRE010, SRE007, and SRE008, or a reverse complement thereof, in a linked double-stranded DNA. In some embodiments, SEQ ID NO: 585 can further comprise, at the 5′ end, a sequence comprising SRE010, SRE007, and SRE008, or a reverse complement thereof, in a linked double-stranded DNA. In some embodiments, SEQ ID NO: 586 can further comprise, at the 5′ end, a sequence comprising SRE010, SRE007, and SRE008, or a reverse complement thereof, in a linked double-stranded DNA. In some embodiments, SEQ ID NO: 587 can further comprise, at the 5′ end, a sequence comprising SRE010, SRE007, and SRE008, or a reverse complement thereof, in a linked double-stranded DNA. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In an embodiment, PR181 can further comprise, at the 5′ end, a sequence comprising SRE010, SRE012, and SRE007, or a reverse complement thereof, in a vector. In some embodiments, PR180 can further comprise, at the 5′ end, a sequence comprising SRE010, SRE012, and SRE007, or a reverse complement thereof, in a vector. In some embodiments, PR179 can further comprise, at the 5′ end, a sequence comprising SRE010, SRE012, and SRE007, or a reverse complement thereof, in a vector. In some embodiments, PR178 can further comprise, at the 5′ end, a sequence comprising SRE010, SRE012, and SRE007, or a reverse complement thereof, in a vector. In some embodiments, PR177 can further comprise, at the 5′ end, a sequence comprising SRE010, SRE012, and SRE007, or a reverse complement thereof, in a vector. In some embodiments, PR176 can further comprise, at the 5′ end, a sequence comprising SRE010, SRE012, and SRE007, or a reverse complement thereof, in a vector. In some embodiments, PR175 can further comprise, at the 5′ end, a sequence comprising SRE010, SRE012, and SRE007, or a reverse complement thereof, in a vector. In some embodiments, PR174 can further comprise, at the 5′ end, a sequence comprising SRE010, SRE012, and SRE007, or a reverse complement thereof, in a vector. In some embodiments, PR173 can further comprise, at the 5′ end, a sequence comprising SRE010, SRE012, and SRE007, or a reverse complement thereof, in a vector. In some embodiments, PR172 can further comprise, at the 5′ end, a sequence comprising SRE010, SRE012, and SRE007, or a reverse complement thereof, in a vector. In some embodiments, PR171 can further comprise, at the 5′ end, a sequence comprising SRE010, SRE012, and SRE007, or a reverse complement thereof, in a vector. In some embodiments, PR170 can further comprise, at the 5′ end, a sequence comprising SRE010, SRE012, and SRE007, or a reverse complement thereof, in a vector. In some embodiments, PR169 can further comprise, at the 5′ end, a sequence comprising SRE010, SRE012 and SRE007, or a reverse complement thereof, in a vector. In some embodiments, PR168 can further comprise, at the 5′ end, a sequence comprising SRE010, SRE012, and SRE007, or a reverse complement thereof, in a vector. In some embodiments, PR167 can further comprise, at the 5′ end, a sequence comprising SRE010, SRE012, and SRE007, or a reverse complement thereof, in a vector. In some embodiments, PR166 can further comprise, at the 5′ end, a sequence comprising SRE010, SRE012, and SRE007, or a reverse complement thereof, in a vector. In some embodiments, PR165 can further comprise, at the 5′ end, a sequence comprising SRE010, SRE012, and SRE007, or a reverse complement thereof, in a vector. In some embodiments, PR159 can further comprise, at the 5′ end, a sequence comprising SRE010, SRE012, and SRE007, or a reverse complement thereof, in a vector. In some embodiments, PR156 can further comprise, at the 5′ end, a sequence comprising SRE010, SRE012, and SRE007, or a reverse complement thereof, in a vector. In some embodiments, PR155 can further comprise, at the 5′ end, a sequence comprising SRE010, SRE012, and SRE007, or a reverse complement thereof, in a vector. In some embodiments, PR154 can further comprise, at the 5′ end, a sequence comprising SRE010, SRE012, and SRE007, or a reverse complement thereof, in a vector. In some embodiments, PR153 can further comprise, at the 5′ end, a sequence comprising SRE010, SRE012, and SRE007, or a reverse complement thereof, in a vector. In some embodiments, PR152 can further comprise, at the 5′ end, a sequence comprising SRE010, SRE012, and SRE007, or a reverse complement thereof, in a vector. In some embodiments, PR151 can further comprise, at the 5′ end, a sequence comprising SRE010, SRE012, and SRE007, or a reverse complement thereof, in a vector. In some embodiments, PR150 can further comprise, at the 5′ end, a sequence comprising SRE010, SRE012, and SRE007, or a reverse complement thereof, in a vector. In some embodiments, PR131 can further comprise, at the 5′ end, a sequence comprising SRE010, SRE012, and SRE007, or a reverse complement thereof, in a vector. In some embodiments, SEQ ID NO: 584 can further comprise, at the 5′ end, a sequence comprising SRE010, SRE012, and SRE007, or a reverse complement thereof, in a vector. In some embodiments, SEQ ID NO: 585 can further comprise, at the 5′ end, a sequence comprising SRE010, SRE012, and SRE007, or a reverse complement thereof, in a vector. In some embodiments, SEQ ID NO: 586 can further comprise, at the 5′ end, a sequence comprising SRE010, SRE012, and SRE007, or a reverse complement thereof, in a vector. In some embodiments, SEQ ID NO: 587 can further comprise, at the 5′ end, a sequence comprising SRE010, SRE012, and SRE007, or a reverse complement thereof, in a vector. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In an embodiment, PR181 can further comprise, at the 5′ end, a sequence comprising SRE010, SRE012, and SRE007, or a reverse complement thereof, in a nanoplasmid. In some embodiments, PR180 can further comprise, at the 5′ end, a sequence comprising SRE010, SRE012, and SRE007, or a reverse complement thereof, in a nanoplasmid. In some embodiments, PR179 can further comprise, at the 5′ end, a sequence comprising SRE010, SRE012, and SRE007, or a reverse complement thereof, in a nanoplasmid. In some embodiments, PR178 can further comprise, at the 5′ end, a sequence comprising SRE010, SRE012, and SRE007, or a reverse complement thereof, in a nanoplasmid. In some embodiments, PR177 can further comprise, at the 5′ end, a sequence comprising SRE010, SRE012, and SRE007, or a reverse complement thereof, in a nanoplasmid. In some embodiments, PR176 can further comprise, at the 5′ end, a sequence comprising SRE010, SRE012, and SRE007, or a reverse complement thereof, in a nanoplasmid. In some embodiments, PR175 can further comprise, at the 5′ end, a sequence comprising SRE010, SRE012, and SRE007, or a reverse complement thereof, in a nanoplasmid. In some embodiments, PR174 can further comprise, at the 5′ end, a sequence comprising SRE010, SRE012, and SRE007, or a reverse complement thereof, in a nanoplasmid. In some embodiments, PR173 can further comprise, at the 5′ end, a sequence comprising SRE010, SRE012, and SRE007, or a reverse complement thereof, in a nanoplasmid. In some embodiments, PR172 can further comprise, at the 5′ end, a sequence comprising SRE010, SRE012, and SRE007, or a reverse complement thereof, in a nanoplasmid. In some embodiments, PR171 can further comprise, at the 5′ end, a sequence comprising SRE010, SRE012, and SRE007, or a reverse complement thereof, in a nanoplasmid. In some embodiments, PR170 can further comprise, at the 5′ end, a sequence comprising SRE010, SRE012, and SRE007, or a reverse complement thereof, in a nanoplasmid. In some embodiments, PR169 can further comprise, at the 5′ end, a sequence comprising SRE010, SRE012 and SRE007, or a reverse complement thereof, in a nanoplasmid. In some embodiments, PR168 can further comprise, at the 5′ end, a sequence comprising SRE010, SRE012, and SRE007, or a reverse complement thereof, in a nanoplasmid. In some embodiments, PR167 can further comprise, at the 5′ end, a sequence comprising SRE010, SRE012, and SRE007, or a reverse complement thereof, in a nanoplasmid. In some embodiments, PR166 can further comprise, at the 5′ end, a sequence comprising SRE010, SRE012, and SRE007, or a reverse complement thereof, in a nanoplasmid. In some embodiments, PR165 can further comprise, at the 5′ end, a sequence comprising SRE010, SRE012, and SRE007, or a reverse complement thereof, in a nanoplasmid. In some embodiments, PR159 can further comprise, at the 5′ end, a sequence comprising SRE010, SRE012, and SRE007, or a reverse complement thereof, in a nanoplasmid. In some embodiments, PR156 can further comprise, at the 5′ end, a sequence comprising SRE010, SRE012, and SRE007, or a reverse complement thereof, in a nanoplasmid. In some embodiments, PR155 can further comprise, at the 5′ end, a sequence comprising SRE010, SRE012, and SRE007, or a reverse complement thereof, in a nanoplasmid. In some embodiments, PR154 can further comprise, at the 5′ end, a sequence comprising SRE010, SRE012, and SRE007, or a reverse complement thereof, in a nanoplasmid. In some embodiments, PR153 can further comprise, at the 5′ end, a sequence comprising SRE010, SRE012, and SRE007, or a reverse complement thereof, in a nanoplasmid. In some embodiments, PR152 can further comprise, at the 5′ end, a sequence comprising SRE010, SRE012, and SRE007, or a reverse complement thereof, in a nanoplasmid. In some embodiments, PR151 can further comprise, at the 5′ end, a sequence comprising SRE010, SRE012, and SRE007, or a reverse complement thereof, in a nanoplasmid. In some embodiments, PR150 can further comprise, at the 5′ end, a sequence comprising SRE010, SRE012, and SRE007, or a reverse complement thereof, in a nanoplasmid. In some embodiments, PR131 can further comprise, at the 5′ end, a sequence comprising SRE010, SRE012, and SRE007, or a reverse complement thereof, in a nanoplasmid. In some embodiments, SEQ ID NO: 584 can further comprise, at the 5′ end, a sequence comprising SRE010, SRE012, and SRE007, or a reverse complement thereof, in a nanoplasmid. In some embodiments, SEQ ID NO: 585 can further comprise, at the 5′ end, a sequence comprising SRE010, SRE012, and SRE007, or a reverse complement thereof, in a nanoplasmid. In some embodiments, SEQ ID NO: 586 can further comprise, at the 5′ end, a sequence comprising SRE010, SRE012, and SRE007, or a reverse complement thereof, in a nanoplasmid. In some embodiments, SEQ ID NO: 587 can further comprise, at the 5′ end, a sequence comprising SRE010, SRE012, and SRE007, or a reverse complement thereof, in a nanoplasmid. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In an embodiment, PR181 can further comprise, at the 5′ end, a sequence comprising SRE010, SRE012, and SRE007, or a reverse complement thereof, in a linked double-stranded DNA. In some embodiments, PR180 can further comprise, at the 5′ end, a sequence comprising SRE010, SRE012, and SRE007, or a reverse complement thereof, in a linked double-stranded DNA. In some embodiments, PR179 can further comprise, at the 5′ end, a sequence comprising SRE010, SRE012, and SRE007, or a reverse complement thereof, in a linked double-stranded DNA. In some embodiments, PR178 can further comprise, at the 5′ end, a sequence comprising SRE010, SRE012, and SRE007, or a reverse complement thereof, in a linked double-stranded DNA. In some embodiments, PR177 can further comprise, at the 5′ end, a sequence comprising SRE010, SRE012, and SRE007, or a reverse complement thereof, in a linked double-stranded DNA. In some embodiments, PR176 can further comprise, at the 5′ end, a sequence comprising SRE010, SRE012, and SRE007, or a reverse complement thereof, in a linked double-stranded DNA. In some embodiments, PR175 can further comprise, at the 5′ end, a sequence comprising SRE010, SRE012, and SRE007, or a reverse complement thereof, in a linked double-stranded DNA. In some embodiments, PR174 can further comprise, at the 5′ end, a sequence comprising SRE010, SRE012, and SRE007, or a reverse complement thereof, in a linked double-stranded DNA. In some embodiments, PR173 can further comprise, at the 5′ end, a sequence comprising SRE010, SRE012, and SRE007, or a reverse complement thereof, in a linked double-stranded DNA. In some embodiments, PR172 can further comprise, at the 5′ end, a sequence comprising SRE010, SRE012, and SRE007, or a reverse complement thereof, in a linked double-stranded DNA. In some embodiments, PR171 can further comprise, at the 5′ end, a sequence comprising SRE010, SRE012, and SRE007, or a reverse complement thereof, in a linked double-stranded DNA. In some embodiments, PR170 can further comprise, at the 5′ end, a sequence comprising SRE010, SRE012, and SRE007, or a reverse complement thereof, in a linked double-stranded DNA. In some embodiments, PR169 can further comprise, at the 5′ end, a sequence comprising SRE010, SRE012 and SRE007, or a reverse complement thereof, in a linked double-stranded DNA. In some embodiments, PR168 can further comprise, at the 5′ end, a sequence comprising SRE010, SRE012, and SRE007, or a reverse complement thereof, in a linked double-stranded DNA. In some embodiments, PR167 can further comprise, at the 5′ end, a sequence comprising SRE010, SRE012, and SRE007, or a reverse complement thereof, in a linked double-stranded DNA. In some embodiments, PR166 can further comprise, at the 5′ end, a sequence comprising SRE010, SRE012, and SRE007, or a reverse complement thereof, in a linked double-stranded DNA. In some embodiments, PR165 can further comprise, at the 5′ end, a sequence comprising SRE010, SRE012, and SRE007, or a reverse complement thereof, in a linked double-stranded DNA. In some embodiments, PR159 can further comprise, at the 5′ end, a sequence comprising SRE010, SRE012, and SRE007, or a reverse complement thereof, in a linked double-stranded DNA. In some embodiments, PR156 can further comprise, at the 5′ end, a sequence comprising SRE010, SRE012, and SRE007, or a reverse complement thereof, in a linked double-stranded DNA. In some embodiments, PR155 can further comprise, at the 5′ end, a sequence comprising SRE010, SRE012, and SRE007, or a reverse complement thereof, in a linked double-stranded DNA. In some embodiments, PR154 can further comprise, at the 5′ end, a sequence comprising SRE010, SRE012, and SRE007, or a reverse complement thereof, in a linked double-stranded DNA. In some embodiments, PR153 can further comprise, at the 5′ end, a sequence comprising SRE010, SRE012, and SRE007, or a reverse complement thereof, in a linked double-stranded DNA. In some embodiments, PR152 can further comprise, at the 5′ end, a sequence comprising SRE010, SRE012, and SRE007, or a reverse complement thereof, in a linked double-stranded DNA. In some embodiments, PR151 can further comprise, at the 5′ end, a sequence comprising SRE010, SRE012, and SRE007, or a reverse complement thereof, in a linked double-stranded DNA. In some embodiments, PR150 can further comprise, at the 5′ end, a sequence comprising SRE010, SRE012, and SRE007, or a reverse complement thereof, in a linked double-stranded DNA. In some embodiments, PR131 can further comprise, at the 5′ end, a sequence comprising SRE010, SRE012, and SRE007, or a reverse complement thereof, in a linked double-stranded DNA. In some embodiments, SEQ ID NO: 584 can further comprise, at the 5′ end, a sequence comprising SRE010, SRE012, and SRE007, or a reverse complement thereof, in a linked double-stranded DNA. In some embodiments, SEQ ID NO: 585 can further comprise, at the 5′ end, a sequence comprising SRE010, SRE012, and SRE007, or a reverse complement thereof, in a linked double-stranded DNA. In some embodiments, SEQ ID NO: 586 can further comprise, at the 5′ end, a sequence comprising SRE010, SRE012, and SRE007, or a reverse complement thereof, in a linked double-stranded DNA. In some embodiments, SEQ ID NO: 587 can further comprise, at the 5′ end, a sequence comprising SRE010, SRE012, and SRE007, or a reverse complement thereof, in a linked double-stranded DNA. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In an embodiment, PR181 can further comprise, at the 5′ end, a sequence comprising SRE010, SRE012, and SRE008, or a reverse complement thereof, in a vector. In some embodiments, PR180 can further comprise, at the 5′ end, a sequence comprising SRE010, SRE012, and SRE008, or a reverse complement thereof, in a vector. In some embodiments, PR179 can further comprise, at the 5′ end, a sequence comprising SRE010, SRE012, and SRE008, or a reverse complement thereof, in a vector. In some embodiments, PR178 can further comprise, at the 5′ end, a sequence comprising SRE010, SRE012, and SRE008, or a reverse complement thereof, in a vector. In some embodiments, PR177 can further comprise, at the 5′ end, a sequence comprising SRE010, SRE012, and SRE008, or a reverse complement thereof, in a vector. In some embodiments, PR176 can further comprise, at the 5′ end, a sequence comprising SRE010, SRE012, and SRE008, or a reverse complement thereof, in a vector. In some embodiments, PR175 can further comprise, at the 5′ end, a sequence comprising SRE010, SRE012, and SRE008, or a reverse complement thereof, in a vector. In some embodiments, PR174 can further comprise, at the 5′ end, a sequence comprising SRE010, SRE012, and SRE008, or a reverse complement thereof, in a vector. In some embodiments, PR173 can further comprise, at the 5′ end, a sequence comprising SRE010, SRE012, and SRE008, or a reverse complement thereof, in a vector. In some embodiments, PR172 can further comprise, at the 5′ end, a sequence comprising SRE010, SRE012, and SRE008, or a reverse complement thereof, in a vector. In some embodiments, PR171 can further comprise, at the 5′ end, a sequence comprising SRE010, SRE012, and SRE008, or a reverse complement thereof, in a vector. In some embodiments, PR170 can further comprise, at the 5′ end, a sequence comprising SRE010, SRE012, and SRE008, or a reverse complement thereof, in a vector. In some embodiments, PR169 can further comprise, at the 5′ end, a sequence comprising SRE010, SRE012 and SRE008, or a reverse complement thereof, in a vector. In some embodiments, PR168 can further comprise, at the 5′ end, a sequence comprising SRE010, SRE012, and SRE008, or a reverse complement thereof, in a vector. In some embodiments, PR167 can further comprise, at the 5′ end, a sequence comprising SRE010, SRE012, and SRE008, or a reverse complement thereof, in a vector. In some embodiments, PR166 can further comprise, at the 5′ end, a sequence comprising SRE010, SRE012, and SRE008, or a reverse complement thereof, in a vector. In some embodiments, PR165 can further comprise, at the 5′ end, a sequence comprising SRE010, SRE012, and SRE008, or a reverse complement thereof, in a vector. In some embodiments, PR159 can further comprise, at the 5′ end, a sequence comprising SRE010, SRE012, and SRE008, or a reverse complement thereof, in a vector. In some embodiments, PR156 can further comprise, at the 5′ end, a sequence comprising SRE010, SRE012, and SRE008, or a reverse complement thereof, in a vector. In some embodiments, PR155 can further comprise, at the 5′ end, a sequence comprising SRE010, SRE012, and SRE008, or a reverse complement thereof, in a vector. In some embodiments, PR154 can further comprise, at the 5′ end, a sequence comprising SRE010, SRE012, and SRE008, or a reverse complement thereof, in a vector. In some embodiments, PR153 can further comprise, at the 5′ end, a sequence comprising SRE010, SRE012, and SRE008, or a reverse complement thereof, in a vector. In some embodiments, PR152 can further comprise, at the 5′ end, a sequence comprising SRE010, SRE012, and SRE008, or a reverse complement thereof, in a vector. In some embodiments, PR151 can further comprise, at the 5′ end, a sequence comprising SRE010, SRE012, and SRE008, or a reverse complement thereof, in a vector. In some embodiments, PR150 can further comprise, at the 5′ end, a sequence comprising SRE010, SRE012, and SRE008, or a reverse complement thereof, in a vector. In some embodiments, PR131 can further comprise, at the 5′ end, a sequence comprising SRE010, SRE012, and SRE008, or a reverse complement thereof, in a vector. In some embodiments, SEQ ID NO: 584 can further comprise, at the 5′ end, a sequence comprising SRE010, SRE012, and SRE008, or a reverse complement thereof, in a vector. In some embodiments, SEQ ID NO: 585 can further comprise, at the 5′ end, a sequence comprising SRE010, SRE012, and SRE008, or a reverse complement thereof, in a vector. In some embodiments, SEQ ID NO: 586 can further comprise, at the 5′ end, a sequence comprising SRE010, SRE012, and SRE008, or a reverse complement thereof, in a vector. In some embodiments, SEQ ID NO: 587 can further comprise, at the 5′ end, a sequence comprising SRE010, SRE012, and SRE008, or a reverse complement thereof, in a vector. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In an embodiment, PR181 can further comprise, at the 5′ end, a sequence comprising SRE010, SRE012, and SRE008, or a reverse complement thereof, in a nanoplasmid. In some embodiments, PR180 can further comprise, at the 5′ end, a sequence comprising SRE010, SRE012, and SRE008, or a reverse complement thereof, in a nanoplasmid. In some embodiments, PR179 can further comprise, at the 5′ end, a sequence comprising SRE010, SRE012, and SRE008, or a reverse complement thereof, in a nanoplasmid. In some embodiments, PR178 can further comprise, at the 5′ end, a sequence comprising SRE010, SRE012, and SRE008, or a reverse complement thereof, in a nanoplasmid. In some embodiments, PR177 can further comprise, at the 5′ end, a sequence comprising SRE010, SRE012, and SRE008, or a reverse complement thereof, in a nanoplasmid. In some embodiments, PR176 can further comprise, at the 5′ end, a sequence comprising SRE010, SRE012, and SRE008, or a reverse complement thereof, in a nanoplasmid. In some embodiments, PR175 can further comprise, at the 5′ end, a sequence comprising SRE010, SRE012, and SRE008, or a reverse complement thereof, in a nanoplasmid. In some embodiments, PR174 can further comprise, at the 5′ end, a sequence comprising SRE010, SRE012, and SRE008, or a reverse complement thereof, in a nanoplasmid. In some embodiments, PR173 can further comprise, at the 5′ end, a sequence comprising SRE010, SRE012, and SRE008, or a reverse complement thereof, in a nanoplasmid. In some embodiments, PR172 can further comprise, at the 5′ end, a sequence comprising SRE010, SRE012, and SRE008, or a reverse complement thereof, in a nanoplasmid. In some embodiments, PR171 can further comprise, at the 5′ end, a sequence comprising SRE010, SRE012, and SRE008, or a reverse complement thereof, in a nanoplasmid. In some embodiments, PR170 can further comprise, at the 5′ end, a sequence comprising SRE010, SRE012, and SRE008, or a reverse complement thereof, in a nanoplasmid. In some embodiments, PR169 can further comprise, at the 5′ end, a sequence comprising SRE010, SRE012 and SRE008, or a reverse complement thereof, in a nanoplasmid. In some embodiments, PR168 can further comprise, at the 5′ end, a sequence comprising SRE010, SRE012, and SRE008, or a reverse complement thereof, in a nanoplasmid. In some embodiments, PR167 can further comprise, at the 5′ end, a sequence comprising SRE010, SRE012, and SRE008, or a reverse complement thereof, in a nanoplasmid. In some embodiments, PR166 can further comprise, at the 5′ end, a sequence comprising SRE010, SRE012, and SRE008, or a reverse complement thereof, in a nanoplasmid. In some embodiments, PR165 can further comprise, at the 5′ end, a sequence comprising SRE010, SRE012, and SRE008, or a reverse complement thereof, in a nanoplasmid. In some embodiments, PR159 can further comprise, at the 5′ end, a sequence comprising SRE010, SRE012, and SRE008, or a reverse complement thereof, in a nanoplasmid. In some embodiments, PR156 can further comprise, at the 5′ end, a sequence comprising SRE010, SRE012, and SRE008, or a reverse complement thereof, in a nanoplasmid. In some embodiments, PR155 can further comprise, at the 5′ end, a sequence comprising SRE010, SRE012, and SRE008, or a reverse complement thereof, in a nanoplasmid. In some embodiments, PR154 can further comprise, at the 5′ end, a sequence comprising SRE010, SRE012, and SRE008, or a reverse complement thereof, in a nanoplasmid. In some embodiments, PR153 can further comprise, at the 5′ end, a sequence comprising SRE010, SRE012, and SRE008, or a reverse complement thereof, in a nanoplasmid. In some embodiments, PR152 can further comprise, at the 5′ end, a sequence comprising SRE010, SRE012, and SRE008, or a reverse complement thereof, in a nanoplasmid. In some embodiments, PR151 can further comprise, at the 5′ end, a sequence comprising SRE010, SRE012, and SRE008, or a reverse complement thereof, in a nanoplasmid. In some embodiments, PR150 can further comprise, at the 5′ end, a sequence comprising SRE010, SRE012, and SRE008, or a reverse complement thereof, in a nanoplasmid. In some embodiments, PR131 can further comprise, at the 5′ end, a sequence comprising SRE010, SRE012, and SRE008, or a reverse complement thereof, in a nanoplasmid. In some embodiments, SEQ ID NO: 584 can further comprise, at the 5′ end, a sequence comprising SRE010, SRE012, and SRE008, or a reverse complement thereof, in a nanoplasmid. In some embodiments, SEQ ID NO: 585 can further comprise, at the 5′ end, a sequence comprising SRE010, SRE012, and SRE008, or a reverse complement thereof, in a nanoplasmid. In some embodiments, SEQ ID NO: 586 can further comprise, at the 5′ end, a sequence comprising SRE010, SRE012, and SRE008, or a reverse complement thereof, in a nanoplasmid. In some embodiments, SEQ ID NO: 587 can further comprise, at the 5′ end, a sequence comprising SRE010, SRE012, and SRE008, or a reverse complement thereof, in a nanoplasmid. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In an embodiment, PR181 can further comprise, at the 5′ end, a sequence comprising SRE010, SRE012, and SRE008, or a reverse complement thereof, in a linked double-stranded DNA. In some embodiments, PR180 can further comprise, at the 5′ end, a sequence comprising SRE010, SRE012, and SRE008, or a reverse complement thereof, in a linked double-stranded DNA. In some embodiments, PR179 can further comprise, at the 5′ end, a sequence comprising SRE010, SRE012, and SRE008, or a reverse complement thereof, in a linked double-stranded DNA. In some embodiments, PR178 can further comprise, at the 5′ end, a sequence comprising SRE010, SRE012, and SRE008, or a reverse complement thereof, in a linked double-stranded DNA. In some embodiments, PR177 can further comprise, at the 5′ end, a sequence comprising SRE010, SRE012, and SRE008, or a reverse complement thereof, in a linked double-stranded DNA. In some embodiments, PR176 can further comprise, at the 5′ end, a sequence comprising SRE010, SRE012, and SRE008, or a reverse complement thereof, in a linked double-stranded DNA. In some embodiments, PR175 can further comprise, at the 5′ end, a sequence comprising SRE010, SRE012, and SRE008, or a reverse complement thereof, in a linked double-stranded DNA. In some embodiments, PR174 can further comprise, at the 5′ end, a sequence comprising SRE010, SRE012, and SRE008, or a reverse complement thereof, in a linked double-stranded DNA. In some embodiments, PR173 can further comprise, at the 5′ end, a sequence comprising SRE010, SRE012, and SRE008, or a reverse complement thereof, in a linked double-stranded DNA. In some embodiments, PR172 can further comprise, at the 5′ end, a sequence comprising SRE010, SRE012, and SRE008, or a reverse complement thereof, in a linked double-stranded DNA. In some embodiments, PR171 can further comprise, at the 5′ end, a sequence comprising SRE010, SRE012, and SRE008, or a reverse complement thereof, in a linked double-stranded DNA. In some embodiments, PR170 can further comprise, at the 5′ end, a sequence comprising SRE010, SRE012, and SRE008, or a reverse complement thereof, in a linked double-stranded DNA. In some embodiments, PR169 can further comprise, at the 5′ end, a sequence comprising SRE010, SRE012 and SRE008, or a reverse complement thereof, in a linked double-stranded DNA. In some embodiments, PR168 can further comprise, at the 5′ end, a sequence comprising SRE010, SRE012, and SRE008, or a reverse complement thereof, in a linked double-stranded DNA. In some embodiments, PR167 can further comprise, at the 5′ end, a sequence comprising SRE010, SRE012, and SRE008, or a reverse complement thereof, in a linked double-stranded DNA. In some embodiments, PR166 can further comprise, at the 5′ end, a sequence comprising SRE010, SRE012, and SRE008, or a reverse complement thereof, in a linked double-stranded DNA. In some embodiments, PR165 can further comprise, at the 5′ end, a sequence comprising SRE010, SRE012, and SRE008, or a reverse complement thereof, in a linked double-stranded DNA. In some embodiments, PR159 can further comprise, at the 5′ end, a sequence comprising SRE010, SRE012, and SRE008, or a reverse complement thereof, in a linked double-stranded DNA. In some embodiments, PR156 can further comprise, at the 5′ end, a sequence comprising SRE010, SRE012, and SRE008, or a reverse complement thereof, in a linked double-stranded DNA. In some embodiments, PR155 can further comprise, at the 5′ end, a sequence comprising SRE010, SRE012, and SRE008, or a reverse complement thereof, in a linked double-stranded DNA. In some embodiments, PR154 can further comprise, at the 5′ end, a sequence comprising SRE010, SRE012, and SRE008, or a reverse complement thereof, in a linked double-stranded DNA. In some embodiments, PR153 can further comprise, at the 5′ end, a sequence comprising SRE010, SRE012, and SRE008, or a reverse complement thereof, in a linked double-stranded DNA. In some embodiments, PR152 can further comprise, at the 5′ end, a sequence comprising SRE010, SRE012, and SRE008, or a reverse complement thereof, in a linked double-stranded DNA. In some embodiments, PR151 can further comprise, at the 5′ end, a sequence comprising SRE010, SRE012, and SRE008, or a reverse complement thereof, in a linked double-stranded DNA. In some embodiments, PR150 can further comprise, at the 5′ end, a sequence comprising SRE010, SRE012, and SRE008, or a reverse complement thereof, in a linked double-stranded DNA. In some embodiments, PR131 can further comprise, at the 5′ end, a sequence comprising SRE010, SRE012, and SRE008, or a reverse complement thereof, in a linked double-stranded DNA. In some embodiments, SEQ ID NO: 584 can further comprise, at the 5′ end, a sequence comprising SRE010, SRE012, and SRE008, or a reverse complement thereof, in a linked double-stranded DNA. In some embodiments, SEQ ID NO: 585 can further comprise, at the 5′ end, a sequence comprising SRE010, SRE012, and SRE008, or a reverse complement thereof, in a linked double-stranded DNA. In some embodiments, SEQ ID NO: 586 can further comprise, at the 5′ end, a sequence comprising SRE010, SRE012, and SRE008, or a reverse complement thereof, in a linked double-stranded DNA. In some embodiments, SEQ ID NO: 587 can further comprise, at the 5′ end, a sequence comprising SRE010, SRE012, and SRE008, or a reverse complement thereof, in a linked double-stranded DNA. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE012, SRE007, and SRE008, or a reverse complement thereof, in a vector. In some embodiments, PR180 can further comprise, at the 5′ end, a sequence comprising SRE012, SRE007, and SRE008, or a reverse complement thereof, in a vector. In some embodiments, PR179 can further comprise, at the 5′ end, a sequence comprising SRE012, SRE007, and SRE008, or a reverse complement thereof, in a vector. In some embodiments, PR178 can further comprise, at the 5′ end, a sequence comprising SRE012, SRE007, and SRE008, or a reverse complement thereof, in a vector. In some embodiments, PR177 can further comprise, at the 5′ end, a sequence comprising SRE012, SRE007, and SRE008, or a reverse complement thereof, in a vector. In some embodiments, PR176 can further comprise, at the 5′ end, a sequence comprising SRE012, SRE007, and SRE008, or a reverse complement thereof, in a vector. In some embodiments, PR175 can further comprise, at the 5′ end, a sequence comprising SRE012, SRE007, and SRE008, or a reverse complement thereof, in a vector. In some embodiments, PR174 can further comprise, at the 5′ end, a sequence comprising SRE012, SRE007, and SRE008, or a reverse complement thereof, in a vector. In some embodiments, PR173 can further comprise, at the 5′ end, a sequence comprising SRE012, SRE007, and SRE008, or a reverse complement thereof, in a vector. In some embodiments, PR172 can further comprise, at the 5′ end, a sequence comprising SRE012, SRE007, and SRE008, or a reverse complement thereof, in a vector. In some embodiments, PR171 can further comprise, at the 5′ end, a sequence comprising SRE012, SRE007, and SRE008, or a reverse complement thereof, in a vector. In some embodiments, PR170 can further comprise, at the 5′ end, a sequence comprising SRE012, SRE007, and SRE008, or a reverse complement thereof, in a vector. In some embodiments, PR169 can further comprise, at the 5′ end, a sequence comprising SRE012, SRE007, and SRE008, or a reverse complement thereof, in a vector. In some embodiments, PR168 can further comprise, at the 5′ end, a sequence comprising SRE012, SRE007, and SRE008, or a reverse complement thereof, in a vector. In some embodiments, PR167 can further comprise, at the 5′ end, a sequence comprising SRE012, SRE007, and SRE008, or a reverse complement thereof, in a vector. In some embodiments, PR166 can further comprise, at the 5′ end, a sequence comprising SRE012, SRE007, and SRE008, or a reverse complement thereof, in a vector. In some embodiments, PR165 can further comprise, at the 5′ end, a sequence comprising SRE012, SRE007, and SRE008, or a reverse complement thereof, in a vector. In some embodiments, PR159 can further comprise, at the 5′ end, a sequence comprising SRE012, SRE007, and SRE008, or a reverse complement thereof, in a vector. In some embodiments, PR156 can further comprise, at the 5′ end, a sequence comprising SRE012, SRE007, and SRE008, or a reverse complement thereof, in a vector. In some embodiments, PR155 can further comprise, at the 5′ end, a sequence comprising SRE012, SRE007, and SRE008, or a reverse complement thereof, in a vector. In some embodiments, PR154 can further comprise, at the 5′ end, a sequence comprising SRE012, SRE007, and SRE008, or a reverse complement thereof, in a vector. In some embodiments, PR153 can further comprise, at the 5′ end, a sequence comprising SRE012, SRE007, and SRE008, or a reverse complement thereof, in a vector. In some embodiments, PR152 can further comprise, at the 5′ end, a sequence comprising SRE012, SRE007, and SRE008, or a reverse complement thereof, in a vector. In some embodiments, PR151 can further comprise, at the 5′ end, a sequence comprising SRE012, SRE007, and SRE008, or a reverse complement thereof, in a vector. In some embodiments, PR150 can further comprise, at the 5′ end, a sequence comprising SRE012, SRE007, and SRE008, or a reverse complement thereof, in a vector. In some embodiments, PR131 can further comprise, at the 5′ end, a sequence comprising SRE012, SRE007, and SRE008, or a reverse complement thereof, in a vector. In some embodiments, SEQ ID NO: 584 can further comprise, at the 5′ end, a sequence comprising SRE012, SRE007, and SRE008, or a reverse complement thereof, in a vector. In some embodiments, SEQ ID NO: 585 can further comprise, at the 5′ end, a sequence comprising SRE012, SRE007, and SRE008, or a reverse complement thereof, in a vector. In some embodiments, SEQ ID NO: 586 can further comprise, at the 5′ end, a sequence comprising SRE012, SRE007, and SRE008, or a reverse complement thereof, in a vector. In some embodiments, SEQ ID NO: 587 can further comprise, at the 5′ end, a sequence comprising SRE012, SRE007, and SRE008, or a reverse complement thereof, in a vector. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE012, SRE007, and SRE008, or a reverse complement thereof, in a nanoplasmid. In some embodiments, PR180 can further comprise, at the 5′ end, a sequence comprising SRE012, SRE007, and SRE008, or a reverse complement thereof, in a nanoplasmid. In some embodiments, PR179 can further comprise, at the 5′ end, a sequence comprising SRE012, SRE007, and SRE008, or a reverse complement thereof, in a nanoplasmid. In some embodiments, PR178 can further comprise, at the 5′ end, a sequence comprising SRE012, SRE007, and SRE008, or a reverse complement thereof, in a nanoplasmid. In some embodiments, PR177 can further comprise, at the 5′ end, a sequence comprising SRE012, SRE007, and SRE008, or a reverse complement thereof, in a nanoplasmid. In some embodiments, PR176 can further comprise, at the 5′ end, a sequence comprising SRE012, SRE007, and SRE008, or a reverse complement thereof, in a nanoplasmid. In some embodiments, PR175 can further comprise, at the 5′ end, a sequence comprising SRE012, SRE007, and SRE008, or a reverse complement thereof, in a nanoplasmid. In some embodiments, PR174 can further comprise, at the 5′ end, a sequence comprising SRE012, SRE007, and SRE008, or a reverse complement thereof, in a nanoplasmid. In some embodiments, PR173 can further comprise, at the 5′ end, a sequence comprising SRE012, SRE007, and SRE008, or a reverse complement thereof, in a nanoplasmid. In some embodiments, PR172 can further comprise, at the 5′ end, a sequence comprising SRE012, SRE007, and SRE008, or a reverse complement thereof, in a nanoplasmid. In some embodiments, PR171 can further comprise, at the 5′ end, a sequence comprising SRE012, SRE007, and SRE008, or a reverse complement thereof, in a nanoplasmid. In some embodiments, PR170 can further comprise, at the 5′ end, a sequence comprising SRE012, SRE007, and SRE008, or a reverse complement thereof, in a nanoplasmid. In some embodiments, PR169 can further comprise, at the 5′ end, a sequence comprising SRE012, SRE007, and SRE008, or a reverse complement thereof, in a nanoplasmid. In some embodiments, PR168 can further comprise, at the 5′ end, a sequence comprising SRE012, SRE007, and SRE008, or a reverse complement thereof, in a nanoplasmid. In some embodiments, PR167 can further comprise, at the 5′ end, a sequence comprising SRE012, SRE007, and SRE008, or a reverse complement thereof, in a nanoplasmid. In some embodiments, PR166 can further comprise, at the 5′ end, a sequence comprising SRE012, SRE007, and SRE008, or a reverse complement thereof, in a nanoplasmid. In some embodiments, PR165 can further comprise, at the 5′ end, a sequence comprising SRE012, SRE007, and SRE008, or a reverse complement thereof, in a nanoplasmid. In some embodiments, PR159 can further comprise, at the 5′ end, a sequence comprising SRE012, SRE007, and SRE008, or a reverse complement thereof, in a nanoplasmid. In some embodiments, PR156 can further comprise, at the 5′ end, a sequence comprising SRE012, SRE007, and SRE008, or a reverse complement thereof, in a nanoplasmid. In some embodiments, PR155 can further comprise, at the 5′ end, a sequence comprising SRE012, SRE007, and SRE008, or a reverse complement thereof, in a nanoplasmid. In some embodiments, PR154 can further comprise, at the 5′ end, a sequence comprising SRE012, SRE007, and SRE008, or a reverse complement thereof, in a nanoplasmid. In some embodiments, PR153 can further comprise, at the 5′ end, a sequence comprising SRE012, SRE007, and SRE008, or a reverse complement thereof, in a nanoplasmid. In some embodiments, PR152 can further comprise, at the 5′ end, a sequence comprising SRE012, SRE007, and SRE008, or a reverse complement thereof, in a nanoplasmid. In some embodiments, PR151 can further comprise, at the 5′ end, a sequence comprising SRE012, SRE007, and SRE008, or a reverse complement thereof, in a nanoplasmid. In some embodiments, PR150 can further comprise, at the 5′ end, a sequence comprising SRE012, SRE007, and SRE008, or a reverse complement thereof, in a nanoplasmid. In some embodiments, PR131 can further comprise, at the 5′ end, a sequence comprising SRE012, SRE007, and SRE008, or a reverse complement thereof, in a nanoplasmid. In some embodiments, SEQ ID NO: 584 can further comprise, at the 5′ end, a sequence comprising SRE012, SRE007, and SRE008, or a reverse complement thereof, in a nanoplasmid. In some embodiments, SEQ ID NO: 585 can further comprise, at the 5′ end, a sequence comprising SRE012, SRE007, and SRE008, or a reverse complement thereof, in a nanoplasmid. In some embodiments, SEQ ID NO: 586 can further comprise, at the 5′ end, a sequence comprising SRE012, SRE007, and SRE008, or a reverse complement thereof, in a nanoplasmid. In some embodiments, SEQ ID NO: 587 can further comprise, at the 5′ end, a sequence comprising SRE012, SRE007, and SRE008, or a reverse complement thereof, in a nanoplasmid. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE012, SRE007, and SRE008, or a reverse complement thereof, in a linked double-stranded DNA. In some embodiments, PR180 can further comprise, at the 5′ end, a sequence comprising SRE012, SRE007, and SRE008, or a reverse complement thereof, in a linked double-stranded DNA. In some embodiments, PR179 can further comprise, at the 5′ end, a sequence comprising SRE012, SRE007, and SRE008, or a reverse complement thereof, in a linked double-stranded DNA. In some embodiments, PR178 can further comprise, at the 5′ end, a sequence comprising SRE012, SRE007, and SRE008, or a reverse complement thereof, in a linked double-stranded DNA. In some embodiments, PR177 can further comprise, at the 5′ end, a sequence comprising SRE012, SRE007, and SRE008, or a reverse complement thereof, in a linked double-stranded DNA. In some embodiments, PR176 can further comprise, at the 5′ end, a sequence comprising SRE012, SRE007, and SRE008, or a reverse complement thereof, in a linked double-stranded DNA. In some embodiments, PR175 can further comprise, at the 5′ end, a sequence comprising SRE012, SRE007, and SRE008, or a reverse complement thereof, in a linked double-stranded DNA. In some embodiments, PR174 can further comprise, at the 5′ end, a sequence comprising SRE012, SRE007, and SRE008, or a reverse complement thereof, in a linked double-stranded DNA. In some embodiments, PR173 can further comprise, at the 5′ end, a sequence comprising SRE012, SRE007, and SRE008, or a reverse complement thereof, in a linked double-stranded DNA. In some embodiments, PR172 can further comprise, at the 5′ end, a sequence comprising SRE012, SRE007, and SRE008, or a reverse complement thereof, in a linked double-stranded DNA. In some embodiments, PR171 can further comprise, at the 5′ end, a sequence comprising SRE012, SRE007, and SRE008, or a reverse complement thereof, in a linked double-stranded DNA. In some embodiments, PR170 can further comprise, at the 5′ end, a sequence comprising SRE012, SRE007, and SRE008, or a reverse complement thereof, in a linked double-stranded DNA. In some embodiments, PR169 can further comprise, at the 5′ end, a sequence comprising SRE012, SRE007, and SRE008, or a reverse complement thereof, in a linked double-stranded DNA. In some embodiments, PR168 can further comprise, at the 5′ end, a sequence comprising SRE012, SRE007, and SRE008, or a reverse complement thereof, in a linked double-stranded DNA. In some embodiments, PR167 can further comprise, at the 5′ end, a sequence comprising SRE012, SRE007, and SRE008, or a reverse complement thereof, in a linked double-stranded DNA. In some embodiments, PR166 can further comprise, at the 5′ end, a sequence comprising SRE012, SRE007, and SRE008, or a reverse complement thereof, in a linked double-stranded DNA. In some embodiments, PR165 can further comprise, at the 5′ end, a sequence comprising SRE012, SRE007, and SRE008, or a reverse complement thereof, in a linked double-stranded DNA. In some embodiments, PR159 can further comprise, at the 5′ end, a sequence comprising SRE012, SRE007, and SRE008, or a reverse complement thereof, in a linked double-stranded DNA. In some embodiments, PR156 can further comprise, at the 5′ end, a sequence comprising SRE012, SRE007, and SRE008, or a reverse complement thereof, in a linked double-stranded DNA. In some embodiments, PR155 can further comprise, at the 5′ end, a sequence comprising SRE012, SRE007, and SRE008, or a reverse complement thereof, in a linked double-stranded DNA. In some embodiments, PR154 can further comprise, at the 5′ end, a sequence comprising SRE012, SRE007, and SRE008, or a reverse complement thereof, in a linked double-stranded DNA. In some embodiments, PR153 can further comprise, at the 5′ end, a sequence comprising SRE012, SRE007, and SRE008, or a reverse complement thereof, in a linked double-stranded DNA. In some embodiments, PR152 can further comprise, at the 5′ end, a sequence comprising SRE012, SRE007, and SRE008, or a reverse complement thereof, in a linked double-stranded DNA. In some embodiments, PR151 can further comprise, at the 5′ end, a sequence comprising SRE012, SRE007, and SRE008, or a reverse complement thereof, in a linked double-stranded DNA. In some embodiments, PR150 can further comprise, at the 5′ end, a sequence comprising SRE012, SRE007, and SRE008, or a reverse complement thereof, in a linked double-stranded DNA. In some embodiments, PR131 can further comprise, at the 5′ end, a sequence comprising SRE012, SRE007, and SRE008, or a reverse complement thereof, in a linked double-stranded DNA. In some embodiments, SEQ ID NO: 584 can further comprise, at the 5′ end, a sequence comprising SRE012, SRE007, and SRE008, or a reverse complement thereof, in a linked double-stranded DNA. In some embodiments, SEQ ID NO: 585 can further comprise, at the 5′ end, a sequence comprising SRE012, SRE007, and SRE008, or a reverse complement thereof, in a linked double-stranded DNA. In some embodiments, SEQ ID NO: 586 can further comprise, at the 5′ end, a sequence comprising SRE012, SRE007, and SRE008, or a reverse complement thereof, in a linked double-stranded DNA. In some embodiments, SEQ ID NO: 587 can further comprise, at the 5′ end, a sequence comprising SRE012, SRE007, and SRE008, or a reverse complement thereof, in a linked double-stranded DNA. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE007 and SRE008, or a reverse complement thereof, in a vector. In some embodiments, PR180 can further comprise, at the 5′ end, a sequence comprising SRE007 and SRE008, or a reverse complement thereof, in a vector. In some embodiments, PR179 can further comprise, at the 5′ end, a sequence comprising SRE007 and SRE008, or a reverse complement thereof, in a vector. In some embodiments, PR178 can further comprise, at the 5′ end, a sequence comprising SRE007 and SRE008, or a reverse complement thereof, in a vector. In some embodiments, PR177 can further comprise, at the 5′ end, a sequence comprising SRE007 and SRE008, or a reverse complement thereof, in a vector. In some embodiments, PR176 can further comprise, at the 5′ end, a sequence comprising SRE007 and SRE008, or a reverse complement thereof, in a vector. In some embodiments, PR175 can further comprise, at the 5′ end, a sequence comprising SRE007 and SRE008, or a reverse complement thereof, in a vector. In some embodiments, PR174 can further comprise, at the 5′ end, a sequence comprising SRE007 and SRE008, or a reverse complement thereof, in a vector. In some embodiments, PR173 can further comprise, at the 5′ end, a sequence comprising SRE007 and SRE008, or a reverse complement thereof, in a vector. In some embodiments, PR172 can further comprise, at the 5′ end, a sequence comprising SRE007 and SRE008, or a reverse complement thereof, in a vector. In some embodiments, PR171 can further comprise, at the 5′ end, a sequence comprising SRE007 and SRE008, or a reverse complement thereof, in a vector. In some embodiments, PR170 can further comprise, at the 5′ end, a sequence comprising SRE007 and SRE008, or a reverse complement thereof, in a vector. In some embodiments, PR169 can further comprise, at the 5′ end, a sequence comprising SRE007 and SRE008, or a reverse complement thereof, in a vector. In some embodiments, PR168 can further comprise, at the 5′ end, a sequence comprising SRE007 and SRE008, or a reverse complement thereof, in a vector. In some embodiments, PR167 can further comprise, at the 5′ end, a sequence comprising SRE007 and SRE008, or a reverse complement thereof, in a vector. In some embodiments, PR166 can further comprise, at the 5′ end, a sequence comprising SRE007 and SRE008, or a reverse complement thereof, in a vector. In some embodiments, PR165 can further comprise, at the 5′ end, a sequence comprising SRE007 and SRE008, or a reverse complement thereof, in a vector. In some embodiments, PR159 can further comprise, at the 5′ end, a sequence comprising SRE007 and SRE008, or a reverse complement thereof, in a vector. In some embodiments, PR156 can further comprise, at the 5′ end, a sequence comprising SRE007 and SRE008, or a reverse complement thereof, in a vector. In some embodiments, PR155 can further comprise, at the 5′ end, a sequence comprising SRE007 and SRE008, or a reverse complement thereof, in a vector. In some embodiments, PR154 can further comprise, at the 5′ end, a sequence comprising SRE007 and SRE008, or a reverse complement thereof, in a vector. In some embodiments, PR153 can further comprise, at the 5′ end, a sequence comprising SRE007 and SRE008, or a reverse complement thereof, in a vector. In some embodiments, PR152 can further comprise, at the 5′ end, a sequence comprising SRE007 and SRE008, or a reverse complement thereof, in a vector. In some embodiments, PR151 can further comprise, at the 5′ end, a sequence comprising SRE007 and SRE008, or a reverse complement thereof, in a vector. In some embodiments, PR150 can further comprise, at the 5′ end, a sequence comprising SRE007 and SRE008, or a reverse complement thereof, in a vector. In some embodiments, PR131 can further comprise, at the 5′ end, a sequence comprising SRE007 and SRE008, or a reverse complement thereof, in a vector. In some embodiments, SEQ ID NO: 584 can further comprise, at the 5′ end, a sequence comprising SRE007 and SRE008, or a reverse complement thereof, in a vector. In some embodiments, SEQ ID NO: 585 can further comprise, at the 5′ end, a sequence comprising SRE007 and SRE008, or a reverse complement thereof, in a vector. In some embodiments, SEQ ID NO: 586 can further comprise, at the 5′ end, a sequence comprising SRE007 and SRE008, or a reverse complement thereof, in a vector. In some embodiments, SEQ ID NO: 587 can further comprise, at the 5′ end, a sequence comprising SRE007 and SRE008, or a reverse complement thereof, in a vector. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE007 and SRE008, or a reverse complement thereof, in a nanoplasmid. In some embodiments, PR180 can further comprise, at the 5′ end, a sequence comprising SRE007 and SRE008, or a reverse complement thereof, in a nanoplasmid. In some embodiments, PR179 can further comprise, at the 5′ end, a sequence comprising SRE007 and SRE008, or a reverse complement thereof, in a nanoplasmid. In some embodiments, PR178 can further comprise, at the 5′ end, a sequence comprising SRE007 and SRE008, or a reverse complement thereof, in a nanoplasmid. In some embodiments, PR177 can further comprise, at the 5′ end, a sequence comprising SRE007 and SRE008, or a reverse complement thereof, in a nanoplasmid. In some embodiments, PR176 can further comprise, at the 5′ end, a sequence comprising SRE007 and SRE008, or a reverse complement thereof, in a nanoplasmid. In some embodiments, PR175 can further comprise, at the 5′ end, a sequence comprising SRE007 and SRE008, or a reverse complement thereof, in a nanoplasmid. In some embodiments, PR174 can further comprise, at the 5′ end, a sequence comprising SRE007 and SRE008, or a reverse complement thereof, in a nanoplasmid. In some embodiments, PR173 can further comprise, at the 5′ end, a sequence comprising SRE007 and SRE008, or a reverse complement thereof, in a nanoplasmid. In some embodiments, PR172 can further comprise, at the 5′ end, a sequence comprising SRE007 and SRE008, or a reverse complement thereof, in a nanoplasmid. In some embodiments, PR171 can further comprise, at the 5′ end, a sequence comprising SRE007 and SRE008, or a reverse complement thereof, in a nanoplasmid. In some embodiments, PR170 can further comprise, at the 5′ end, a sequence comprising SRE007 and SRE008, or a reverse complement thereof, in a nanoplasmid. In some embodiments, PR169 can further comprise, at the 5′ end, a sequence comprising SRE007 and SRE008, or a reverse complement thereof, in a nanoplasmid. In some embodiments, PR168 can further comprise, at the 5′ end, a sequence comprising SRE007 and SRE008, or a reverse complement thereof, in a nanoplasmid. In some embodiments, PR167 can further comprise, at the 5′ end, a sequence comprising SRE007 and SRE008, or a reverse complement thereof, in a nanoplasmid. In some embodiments, PR166 can further comprise, at the 5′ end, a sequence comprising SRE007 and SRE008, or a reverse complement thereof, in a nanoplasmid. In some embodiments, PR165 can further comprise, at the 5′ end, a sequence comprising SRE007 and SRE008, or a reverse complement thereof, in a nanoplasmid. In some embodiments, PR159 can further comprise, at the 5′ end, a sequence comprising SRE007 and SRE008, or a reverse complement thereof, in a nanoplasmid. In some embodiments, PR156 can further comprise, at the 5′ end, a sequence comprising SRE007 and SRE008, or a reverse complement thereof, in a nanoplasmid. In some embodiments, PR155 can further comprise, at the 5′ end, a sequence comprising SRE007 and SRE008, or a reverse complement thereof, in a nanoplasmid. In some embodiments, PR154 can further comprise, at the 5′ end, a sequence comprising SRE007 and SRE008, or a reverse complement thereof, in a nanoplasmid. In some embodiments, PR153 can further comprise, at the 5′ end, a sequence comprising SRE007 and SRE008, or a reverse complement thereof, in a nanoplasmid. In some embodiments, PR152 can further comprise, at the 5′ end, a sequence comprising SRE007 and SRE008, or a reverse complement thereof, in a nanoplasmid. In some embodiments, PR151 can further comprise, at the 5′ end, a sequence comprising SRE007 and SRE008, or a reverse complement thereof, in a nanoplasmid. In some embodiments, PR150 can further comprise, at the 5′ end, a sequence comprising SRE007 and SRE008, or a reverse complement thereof, in a nanoplasmid. In some embodiments, PR131 can further comprise, at the 5′ end, a sequence comprising SRE007 and SRE008, or a reverse complement thereof, in a nanoplasmid. In some embodiments, SEQ ID NO: 584 can further comprise, at the 5′ end, a sequence comprising SRE007 and SRE008, or a reverse complement thereof, in a nanoplasmid. In some embodiments, SEQ ID NO: 585 can further comprise, at the 5′ end, a sequence comprising SRE007 and SRE008, or a reverse complement thereof, in a nanoplasmid. In some embodiments, SEQ ID NO: 586 can further comprise, at the 5′ end, a sequence comprising SRE007 and SRE008, or a reverse complement thereof, in a nanoplasmid. In some embodiments, SEQ ID NO: 587 can further comprise, at the 5′ end, a sequence comprising SRE007 and SRE008, or a reverse complement thereof, in a nanoplasmid. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE007 and SRE008, or a reverse complement thereof, in a linked double-stranded DNA. In some embodiments, PR180 can further comprise, at the 5′ end, a sequence comprising SRE007 and SRE008, or a reverse complement thereof, in a linked double-stranded DNA. In some embodiments, PR179 can further comprise, at the 5′ end, a sequence comprising SRE007 and SRE008, or a reverse complement thereof, in a linked double-stranded DNA. In some embodiments, PR178 can further comprise, at the 5′ end, a sequence comprising SRE007 and SRE008, or a reverse complement thereof, in a linked double-stranded DNA. In some embodiments, PR177 can further comprise, at the 5′ end, a sequence comprising SRE007 and SRE008, or a reverse complement thereof, in a linked double-stranded DNA. In some embodiments, PR176 can further comprise, at the 5′ end, a sequence comprising SRE007 and SRE008, or a reverse complement thereof, in a linked double-stranded DNA. In some embodiments, PR175 can further comprise, at the 5′ end, a sequence comprising SRE007 and SRE008, or a reverse complement thereof, in a linked double-stranded DNA. In some embodiments, PR174 can further comprise, at the 5′ end, a sequence comprising SRE007 and SRE008, or a reverse complement thereof, in a linked double-stranded DNA. In some embodiments, PR173 can further comprise, at the 5′ end, a sequence comprising SRE007 and SRE008, or a reverse complement thereof, in a linked double-stranded DNA. In some embodiments, PR172 can further comprise, at the 5′ end, a sequence comprising SRE007 and SRE008, or a reverse complement thereof, in a linked double-stranded DNA. In some embodiments, PR171 can further comprise, at the 5′ end, a sequence comprising SRE007 and SRE008, or a reverse complement thereof, in a linked double-stranded DNA. In some embodiments, PR170 can further comprise, at the 5′ end, a sequence comprising SRE007 and SRE008, or a reverse complement thereof, in a linked double-stranded DNA. In some embodiments, PR169 can further comprise, at the 5′ end, a sequence comprising SRE007 and SRE008, or a reverse complement thereof, in a linked double-stranded DNA. In some embodiments, PR168 can further comprise, at the 5′ end, a sequence comprising SRE007 and SRE008, or a reverse complement thereof, in a linked double-stranded DNA. In some embodiments, PR167 can further comprise, at the 5′ end, a sequence comprising SRE007 and SRE008, or a reverse complement thereof, in a linked double-stranded DNA. In some embodiments, PR166 can further comprise, at the 5′ end, a sequence comprising SRE007 and SRE008, or a reverse complement thereof, in a linked double-stranded DNA. In some embodiments, PR165 can further comprise, at the 5′ end, a sequence comprising SRE007 and SRE008, or a reverse complement thereof, in a linked double-stranded DNA. In some embodiments, PR159 can further comprise, at the 5′ end, a sequence comprising SRE007 and SRE008, or a reverse complement thereof, in a linked double-stranded DNA. In some embodiments, PR156 can further comprise, at the 5′ end, a sequence comprising SRE007 and SRE008, or a reverse complement thereof, in a linked double-stranded DNA. In some embodiments, PR155 can further comprise, at the 5′ end, a sequence comprising SRE007 and SRE008, or a reverse complement thereof, in a linked double-stranded DNA. In some embodiments, PR154 can further comprise, at the 5′ end, a sequence comprising SRE007 and SRE008, or a reverse complement thereof, in a linked double-stranded DNA. In some embodiments, PR153 can further comprise, at the 5′ end, a sequence comprising SRE007 and SRE008, or a reverse complement thereof, in a linked double-stranded DNA. In some embodiments, PR152 can further comprise, at the 5′ end, a sequence comprising SRE007 and SRE008, or a reverse complement thereof, in a linked double-stranded DNA. In some embodiments, PR151 can further comprise, at the 5′ end, a sequence comprising SRE007 and SRE008, or a reverse complement thereof, in a linked double-stranded DNA. In some embodiments, PR150 can further comprise, at the 5′ end, a sequence comprising SRE007 and SRE008, or a reverse complement thereof, in a linked double-stranded DNA. In some embodiments, PR131 can further comprise, at the 5′ end, a sequence comprising SRE007 and SRE008, or a reverse complement thereof, in a linked double-stranded DNA. In some embodiments, SEQ ID NO: 584 can further comprise, at the 5′ end, a sequence comprising SRE007 and SRE008, or a reverse complement thereof, in a linked double-stranded DNA. In some embodiments, SEQ ID NO: 585 can further comprise, at the 5′ end, a sequence comprising SRE007 and SRE008, or a reverse complement thereof, in a linked double-stranded DNA. In some embodiments, SEQ ID NO: 586 can further comprise, at the 5′ end, a sequence comprising SRE007 and SRE008, or a reverse complement thereof, in a linked double-stranded DNA. In some embodiments, SEQ ID NO: 587 can further comprise, at the 5′ end, a sequence comprising SRE007 and SRE008, or a reverse complement thereof, in a linked double-stranded DNA. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE007 and SRE010, or a reverse complement thereof, in a vector. In some embodiments, PR180 can further comprise, at the 5′ end, a sequence comprising SRE007 and SRE010, or a reverse complement thereof, in a vector. In some embodiments, PR179 can further comprise, at the 5′ end, a sequence comprising SRE007 and SRE010, or a reverse complement thereof, in a vector. In some embodiments, PR178 can further comprise, at the 5′ end, a sequence comprising SRE007 and SRE010, or a reverse complement thereof, in a vector. In some embodiments, PR177 can further comprise, at the 5′ end, a sequence comprising SRE007 and SRE010, or a reverse complement thereof, in a vector. In some embodiments, PR176 can further comprise, at the 5′ end, a sequence comprising SRE007 and SRE010, or a reverse complement thereof, in a vector. In some embodiments, PR175 can further comprise, at the 5′ end, a sequence comprising SRE007 and SRE010, or a reverse complement thereof, in a vector. In some embodiments, PR174 can further comprise, at the 5′ end, a sequence comprising SRE007 and SRE010, or a reverse complement thereof, in a vector. In some embodiments, PR173 can further comprise, at the 5′ end, a sequence comprising SRE007 and SRE010, or a reverse complement thereof, in a vector. In some embodiments, PR172 can further comprise, at the 5′ end, a sequence comprising SRE007 and SRE010, or a reverse complement thereof, in a vector. In some embodiments, PR171 can further comprise, at the 5′ end, a sequence comprising SRE007 and SRE010, or a reverse complement thereof, in a vector. In some embodiments, PR170 can further comprise, at the 5′ end, a sequence comprising SRE007 and SRE010, or a reverse complement thereof, in a vector. In some embodiments, PR169 can further comprise, at the 5′ end, a sequence comprising SRE007 and SRE010, or a reverse complement thereof, in a vector. In some embodiments, PR168 can further comprise, at the 5′ end, a sequence comprising SRE007 and SRE010, or a reverse complement thereof, in a vector. In some embodiments, PR167 can further comprise, at the 5′ end, a sequence comprising SRE007 and SRE010, or a reverse complement thereof, in a vector. In some embodiments, PR166 can further comprise, at the 5′ end, a sequence comprising SRE007 and SRE010, or a reverse complement thereof, in a vector. In some embodiments, PR165 can further comprise, at the 5′ end, a sequence comprising SRE007 and SRE010, or a reverse complement thereof, in a vector. In some embodiments, PR159 can further comprise, at the 5′ end, a sequence comprising SRE007 and SRE010, or a reverse complement thereof, in a vector. In some embodiments, PR156 can further comprise, at the 5′ end, a sequence comprising SRE007 and SRE010, or a reverse complement thereof, in a vector. In some embodiments, PR155 can further comprise, at the 5′ end, a sequence comprising SRE007 and SRE010, or a reverse complement thereof, in a vector. In some embodiments, PR154 can further comprise, at the 5′ end, a sequence comprising SRE007 and SRE010, or a reverse complement thereof, in a vector. In some embodiments, PR153 can further comprise, at the 5′ end, a sequence comprising SRE007 and SRE010, or a reverse complement thereof, in a vector. In some embodiments, PR152 can further comprise, at the 5′ end, a sequence comprising SRE007 and SRE010, or a reverse complement thereof, in a vector. In some embodiments, PR151 can further comprise, at the 5′ end, a sequence comprising SRE007 and SRE010, or a reverse complement thereof, in a vector. In some embodiments, PR150 can further comprise, at the 5′ end, a sequence comprising SRE007 and SRE010, or a reverse complement thereof, in a vector. In some embodiments, PR131 can further comprise, at the 5′ end, a sequence comprising SRE007 and SRE010, or a reverse complement thereof, in a vector. In some embodiments, SEQ ID NO: 584 can further comprise, at the 5′ end, a sequence comprising SRE007 and SRE010, or a reverse complement thereof, in a vector. In some embodiments, SEQ ID NO: 585 can further comprise, at the 5′ end, a sequence comprising SRE007 and SRE010, or a reverse complement thereof, in a vector. In some embodiments, SEQ ID NO: 586 can further comprise, at the 5′ end, a sequence comprising SRE007 and SRE010, or a reverse complement thereof, in a vector. In some embodiments, SEQ ID NO: 587 can further comprise, at the 5′ end, a sequence comprising SRE007 and SRE010, or a reverse complement thereof, in a vector. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE007 and SRE010, or a reverse complement thereof, in a nanoplasmid. In some embodiments, PR180 can further comprise, at the 5′ end, a sequence comprising SRE007 and SRE010, or a reverse complement thereof, in a nanoplasmid. In some embodiments, PR179 can further comprise, at the 5′ end, a sequence comprising SRE007 and SRE010, or a reverse complement thereof, in a nanoplasmid. In some embodiments, PR178 can further comprise, at the 5′ end, a sequence comprising SRE007 and SRE010, or a reverse complement thereof, in a nanoplasmid. In some embodiments, PR177 can further comprise, at the 5′ end, a sequence comprising SRE007 and SRE010, or a reverse complement thereof, in a nanoplasmid. In some embodiments, PR176 can further comprise, at the 5′ end, a sequence comprising SRE007 and SRE010, or a reverse complement thereof, in a nanoplasmid. In some embodiments, PR175 can further comprise, at the 5′ end, a sequence comprising SRE007 and SRE010, or a reverse complement thereof, in a nanoplasmid. In some embodiments, PR174 can further comprise, at the 5′ end, a sequence comprising SRE007 and SRE010, or a reverse complement thereof, in a nanoplasmid. In some embodiments, PR173 can further comprise, at the 5′ end, a sequence comprising SRE007 and SRE010, or a reverse complement thereof, in a nanoplasmid. In some embodiments, PR172 can further comprise, at the 5′ end, a sequence comprising SRE007 and SRE010, or a reverse complement thereof, in a SRE007 and SRE010, or a reverse complement thereof, in a nanoplasmid. In some embodiments, PR170 can further comprise, at the 5′ end, a sequence comprising SRE007 and SRE010, or a reverse complement thereof, in a nanoplasmid. In some embodiments, PR169 can further comprise, at the 5′ end, a sequence comprising SRE007 and SRE010, or a reverse complement thereof, in a nanoplasmid. In some embodiments, PR168 can further comprise, at the 5′ end, a sequence comprising SRE007 and SRE010, or a reverse complement thereof, in a nanoplasmid. In some embodiments, PR167 can further comprise, at the 5′ end, a sequence comprising SRE007 and SRE010, or a reverse complement thereof, in a nanoplasmid. In some embodiments, PR166 can further comprise, at the 5′ end, a sequence comprising SRE007 and SRE010, or a reverse complement thereof, in a nanoplasmid. In some embodiments, PR165 can further comprise, at the 5′ end, a sequence comprising SRE007 and SRE010, or a reverse complement thereof, in a nanoplasmid. In some embodiments, PR159 can further comprise, at the 5′ end, a sequence comprising SRE007 and SRE010, or a reverse complement thereof, in a nanoplasmid. In some embodiments, PR156 can further comprise, at the 5′ end, a sequence comprising SRE007 and SRE010, or a reverse complement thereof, in a nanoplasmid. In some embodiments, PR155 can further comprise, at the 5′ end, a sequence comprising SRE007 and SRE010, or a reverse complement thereof, in a nanoplasmid. In some embodiments, PR154 can further comprise, at the 5′ end, a sequence comprising SRE007 and SRE010, or a reverse complement thereof, in a nanoplasmid. In some embodiments, PR153 can further comprise, at the 5′ end, a sequence comprising SRE007 and SRE010, or a reverse complement thereof, in a nanoplasmid. In some embodiments, PR152 can further comprise, at the 5′ end, a sequence comprising SRE007 and SRE010, or a reverse complement thereof, in a nanoplasmid. In some embodiments, PR151 can further comprise, at the 5′ end, a sequence comprising SRE007 and SRE010, or a reverse complement thereof, in a nanoplasmid. In some embodiments, PR150 can further comprise, at the 5′ end, a sequence comprising SRE007 and SRE010, or a reverse complement thereof, in a nanoplasmid. In some embodiments, PR131 can further comprise, at the 5′ end, a sequence comprising SRE007 and SRE010, or a reverse complement thereof, in a nanoplasmid. In some embodiments, SEQ ID NO: 584 can further comprise, at the 5′ end, a sequence comprising SRE007 and SRE010, or a reverse complement thereof, in a nanoplasmid. In some embodiments, SEQ ID NO: 585 can further comprise, at the 5′ end, a sequence comprising SRE007 and SRE010, or a reverse complement thereof, in a nanoplasmid. In some embodiments, SEQ ID NO: 586 can further comprise, at the 5′ end, a sequence comprising SRE007 and SRE010, or a reverse complement thereof, in a nanoplasmid. In some embodiments, SEQ ID NO: 587 can further comprise, at the 5′ end, a sequence comprising SRE007 and SRE010, or a reverse complement thereof, in a nanoplasmid. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE007 and SRE010, or a reverse complement thereof, in a linked double-stranded DNA. In some embodiments, PR180 can further comprise, at the 5′ end, a sequence comprising SRE007 and SRE010, or a reverse complement thereof, in a linked double-stranded DNA. In some embodiments, PR179 can further comprise, at the 5′ end, a sequence comprising SRE007 and SRE010, or a reverse complement thereof, in a linked double-stranded DNA. In some embodiments, PR178 can further comprise, at the 5′ end, a sequence comprising SRE007 and SRE010, or a reverse complement thereof, in a linked double-stranded DNA. In some embodiments, PR177 can further comprise, at the 5′ end, a sequence comprising SRE007 and SRE010, or a reverse complement thereof, in a linked double-stranded DNA. In some embodiments, PR176 can further comprise, at the 5′ end, a sequence comprising SRE007 and SRE010, or a reverse complement thereof, in a linked double-stranded DNA. In some embodiments, PR175 can further comprise, at the 5′ end, a sequence comprising SRE007 and SRE010, or a reverse complement thereof, in a linked double-stranded DNA. In some embodiments, PR174 can further comprise, at the 5′ end, a sequence comprising SRE007 and SRE010, or a reverse complement thereof, in a linked double-stranded DNA. In some embodiments, PR173 can further comprise, at the 5′ end, a sequence comprising SRE007 and SRE010, or a reverse complement thereof, in a linked double-stranded DNA. In some embodiments, PR172 can further comprise, at the 5′ end, a sequence comprising SRE007 and SRE010, or a reverse complement thereof, in a linked double-stranded DNA. In some embodiments, PR171 can further comprise, at the 5′ end, a sequence comprising SRE007 and SRE010, or a reverse complement thereof, in a linked double-stranded DNA. In some embodiments, PR170 can further comprise, at the 5′ end, a sequence comprising SRE007 and SRE010, or a reverse complement thereof, in a linked double-stranded DNA. In some embodiments, PR169 can further comprise, at the 5′ end, a sequence comprising SRE007 and SRE010, or a reverse complement thereof, in a linked double-stranded DNA. In some embodiments, PR168 can further comprise, at the 5′ end, a sequence comprising SRE007 and SRE010, or a reverse complement thereof, in a linked double-stranded DNA. In some embodiments, PR167 can further comprise, at the 5′ end, a sequence comprising SRE007 and SRE010, or a reverse complement thereof, in a linked double-stranded DNA. In some embodiments, PR166 can further comprise, at the 5′ end, a sequence comprising SRE007 and SRE010, or a reverse complement thereof, in a linked double-stranded DNA. In some embodiments, PR165 can further comprise, at the 5′ end, a sequence comprising SRE007 and SRE010, or a reverse complement thereof, in a linked double-stranded DNA. In some embodiments, PR159 can further comprise, at the 5′ end, a sequence comprising SRE007 and SRE010, or a reverse complement thereof, in a linked double-stranded DNA. In some embodiments, PR156 can further comprise, at the 5′ end, a sequence comprising SRE007 and SRE010, or a reverse complement thereof, in a linked double-stranded DNA. In some embodiments, PR155 can further comprise, at the 5′ end, a sequence comprising SRE007 and SRE010, or a reverse complement thereof, in a linked double-stranded DNA. In some embodiments, PR154 can further comprise, at the 5′ end, a sequence comprising SRE007 and SRE010, or a reverse complement thereof, in a linked double-stranded DNA. In some embodiments, PR153 can further comprise, at the 5′ end, a sequence comprising SRE007 and SRE010, or a reverse complement thereof, in a linked double-stranded DNA. In some embodiments, PR152 can further comprise, at the 5′ end, a sequence comprising SRE007 and SRE010, or a reverse complement thereof, in a linked double-stranded DNA. In some embodiments, PR151 can further comprise, at the 5′ end, a sequence comprising SRE007 and SRE010, or a reverse complement thereof, in a linked double-stranded DNA. In some embodiments, PR150 can further comprise, at the 5′ end, a sequence comprising SRE007 and SRE010, or a reverse complement thereof, in a linked double-stranded DNA. In some embodiments, PR131 can further comprise, at the 5′ end, a sequence comprising SRE007 and SRE010, or a reverse complement thereof, in a linked double-stranded DNA. In some embodiments, SEQ ID NO: 584 can further comprise, at the 5′ end, a sequence comprising SRE007 and SRE010, or a reverse complement thereof, in a linked double-stranded DNA. In some embodiments, SEQ ID NO: 585 can further comprise, at the 5′ end, a sequence comprising SRE007 and SRE010, or a reverse complement thereof, in a linked double-stranded DNA. In some embodiments, SEQ ID NO: 586 can further comprise, at the 5′ end, a sequence comprising SRE007 and SRE010, or a reverse complement thereof, in a linked double-stranded DNA. In some embodiments, SEQ ID NO: 587 can further comprise, at the 5′ end, a sequence comprising SRE007 and SRE010, or a reverse complement thereof, in a linked double-stranded DNA. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE007 and SRE012, or a reverse complement thereof, in a vector. In some embodiments, PR180 can further comprise, at the 5′ end, a sequence comprising SRE007 and SRE012, or a reverse complement thereof, in a vector. In some embodiments, PR179 can further comprise, at the 5′ end, a sequence comprising SRE007 and SRE012, or a reverse complement thereof, in a vector. In some embodiments, PR178 can further comprise, at the 5′ end, a sequence comprising SRE007 and SRE012, or a reverse complement thereof, in a vector. In some embodiments, PR177 can further comprise, at the 5′ end, a sequence comprising SRE007 and SRE012, or a reverse complement thereof, in a vector. In some embodiments, PR176 can further comprise, at the 5′ end, a sequence comprising SRE007 and SRE012, or a reverse complement thereof, in a vector. In some embodiments, PR175 can further comprise, at the 5′ end, a sequence comprising SRE007 and SRE012, or a reverse complement thereof, in a vector. In some embodiments, PR174 can further comprise, at the 5′ end, a sequence comprising SRE007 and SRE012, or a reverse complement thereof, in a vector. In some embodiments, PR173 can further comprise, at the 5′ end, a sequence comprising SRE007 and SRE012, or a reverse complement thereof, in a vector. In some embodiments, PR172 can further comprise, at the 5′ end, a sequence comprising SRE007 and SRE012, or a reverse complement thereof, in a vector. In some embodiments, PR171 can further comprise, at the 5′ end, a sequence comprising SRE007 and SRE012, or a reverse complement thereof, in a vector. In some embodiments, PR170 can further comprise, at the 5′ end, a sequence comprising SRE007 and SRE012, or a reverse complement thereof, in a vector. In some embodiments, PR169 can further comprise, at the 5′ end, a sequence comprising SRE007 and SRE012, or a reverse complement thereof, in a vector. In some embodiments, PR168 can further comprise, at the 5′ end, a sequence comprising SRE007 and SRE012, or a reverse complement thereof, in a vector. In some embodiments, PR167 can further comprise, at the 5′ end, a sequence comprising SRE007 and SRE012, or a reverse complement thereof, in a vector. In some embodiments, PR166 can further comprise, at the 5′ end, a sequence comprising SRE007 and SRE012, or a reverse complement thereof, in a vector. In some embodiments, PR165 can further comprise, at the 5′ end, a sequence comprising SRE007 and SRE012, or a reverse complement thereof, in a vector. In some embodiments, PR159 can further comprise, at the 5′ end, a sequence comprising SRE007 and SRE012, or a reverse complement thereof, in a vector. In some embodiments, PR156 can further comprise, at the 5′ end, a sequence comprising SRE007 and SRE012, or a reverse complement thereof, in a vector. In some embodiments, PR155 can further comprise, at the 5′ end, a sequence comprising SRE007 and SRE012, or a reverse complement thereof, in a vector. In some embodiments, PR154 can further comprise, at the 5′ end, a sequence comprising SRE007 and SRE012, or a reverse complement thereof, in a vector. In some embodiments, PR153 can further comprise, at the 5′ end, a sequence comprising SRE007 and SRE012, or a reverse complement thereof, in a vector. In some embodiments, PR152 can further comprise, at the 5′ end, a sequence comprising SRE007 and SRE012, or a reverse complement thereof, in a vector. In some embodiments, PR151 can further comprise, at the 5′ end, a sequence comprising SRE007 and SRE012, or a reverse complement thereof, in a vector. In some embodiments, PR150 can further comprise, at the 5′ end, a sequence comprising SRE007 and SRE012, or a reverse complement thereof, in a vector. In some embodiments, PR131 can further comprise, at the 5′ end, a sequence comprising SRE007 and SRE012, or a reverse complement thereof, in a vector. In some embodiments, SEQ ID NO: 584 can further comprise, at the 5′ end, a sequence comprising SRE007 and SRE012, or a reverse complement thereof, in a vector. In some embodiments, SEQ ID NO: 585 can further comprise, at the 5′ end, a sequence comprising SRE007 and SRE012, or a reverse complement thereof, in a vector. In some embodiments, SEQ ID NO: 586 can further comprise, at the 5′ end, a sequence comprising SRE007 and SRE012, or a reverse complement thereof, in a vector. In some embodiments, SEQ ID NO: 587 can further comprise, at the 5′ end, a sequence comprising SRE007 and SRE012, or a reverse complement thereof, in a vector. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE007 and SRE012, or a reverse complement thereof, in a nanoplasmid. In some embodiments, PR180 can further comprise, at the 5′ end, a sequence comprising SRE007 and SRE012, or a reverse complement thereof, in a nanoplasmid. In some embodiments, PR179 can further comprise, at the 5′ end, a sequence comprising SRE007 and SRE012, or a reverse complement thereof, in a nanoplasmid. In some embodiments, PR178 can further comprise, at the 5′ end, a sequence comprising SRE007 and SRE012, or a reverse complement thereof, in a nanoplasmid. In some embodiments, PR177 can further comprise, at the 5′ end, a sequence comprising SRE007 and SRE012, or a reverse complement thereof, in a nanoplasmid. In some embodiments, PR176 can further comprise, at the 5′ end, a sequence comprising SRE007 and SRE012, or a reverse complement thereof, in a nanoplasmid. In some embodiments, PR175 can further comprise, at the 5′ end, a sequence comprising SRE007 and SRE012, or a reverse complement thereof, in a nanoplasmid. In some embodiments, PR174 can further comprise, at the 5′ end, a sequence comprising SRE007 and SRE012, or a reverse complement thereof, in a nanoplasmid. In some embodiments, PR173 can further comprise, at the 5′ end, a sequence comprising SRE007 and SRE012, or a reverse complement thereof, in a nanoplasmid. In some embodiments, PR172 can further comprise, at the 5′ end, a sequence comprising SRE007 and SRE012, or a reverse complement thereof, in a nanoplasmid. In some embodiments, PR171 can further comprise, at the 5′ end, a sequence comprising SRE007 and SRE012, or a reverse complement thereof, in a nanoplasmid. In some embodiments, PR170 can further comprise, at the 5′ end, a sequence comprising SRE007 and SRE012, or a reverse complement thereof, in a nanoplasmid. In some embodiments, PR169 can further comprise, at the 5′ end, a sequence comprising SRE007 and SRE012, or a reverse complement thereof, in a nanoplasmid. In some embodiments, PR168 can further comprise, at the 5′ end, a sequence comprising SRE007 and SRE012, or a reverse complement thereof, in a nanoplasmid. In some embodiments, PR167 can further comprise, at the 5′ end, a sequence comprising SRE007 and SRE012, or a reverse complement thereof, in a nanoplasmid. In some embodiments, PR166 can further comprise, at the 5′ end, a sequence comprising SRE007 and SRE012, or a reverse complement thereof, in a nanoplasmid. In some embodiments, PR165 can further comprise, at the 5′ end, a sequence comprising SRE007 and SRE012, or a reverse complement thereof, in a nanoplasmid. In some embodiments, PR159 can further comprise, at the 5′ end, a sequence comprising SRE007 and SRE012, or a reverse complement thereof, in a nanoplasmid. In some embodiments, PR156 can further comprise, at the 5′ end, a sequence comprising SRE007 and SRE012, or a reverse complement thereof, in a nanoplasmid. In some embodiments, PR155 can further comprise, at the 5′ end, a sequence comprising SRE007 and SRE012, or a reverse complement thereof, in a nanoplasmid. In some embodiments, PR154 can further comprise, at the 5′ end, a sequence comprising SRE007 and SRE012, or a reverse complement thereof, in a nanoplasmid. In some embodiments, PR153 can further comprise, at the 5′ end, a sequence comprising SRE007 and SRE012, or a reverse complement thereof, in a nanoplasmid. In some embodiments, PR152 can further comprise, at the 5′ end, a sequence comprising SRE007 and SRE012, or a reverse complement thereof, in a nanoplasmid. In some embodiments, PR151 can further comprise, at the 5′ end, a sequence comprising SRE007 and SRE012, or a reverse complement thereof, in a nanoplasmid. In some embodiments, PR150 can further comprise, at the 5′ end, a sequence comprising SRE007 and SRE012, or a reverse complement thereof, in a nanoplasmid. In some embodiments, PR131 can further comprise, at the 5′ end, a sequence comprising SRE007 and SRE012, or a reverse complement thereof, in a nanoplasmid. In some embodiments, SEQ ID NO: 584 can further comprise, at the 5′ end, a sequence comprising SRE007 and SRE012, or a reverse complement thereof, in a nanoplasmid. In some embodiments, SEQ ID NO: 585 can further comprise, at the 5′ end, a sequence comprising SRE007 and SRE012, or a reverse complement thereof, in a nanoplasmid. In some embodiments, SEQ ID NO: 586 can further comprise, at the 5′ end, a sequence comprising SRE007 and SRE012, or a reverse complement thereof, in a nanoplasmid. In some embodiments, SEQ ID NO: 587 can further comprise, at the 5′ end, a sequence comprising SRE007 and SRE012, or a reverse complement thereof, in a nanoplasmid. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE007 and SRE012, or a reverse complement thereof, in a linked double-stranded DNA. In some embodiments, PR180 can further comprise, at the 5′ end, a sequence comprising SRE007 and SRE012, or a reverse complement thereof, in a linked double-stranded DNA. In some embodiments, PR179 can further comprise, at the 5′ end, a sequence comprising SRE007 and SRE012, or a reverse complement thereof, in a linked double-stranded DNA. In some embodiments, PR178 can further comprise, at the 5′ end, a sequence comprising SRE007 and SRE012, or a reverse complement thereof, in a linked double-stranded DNA. In some embodiments, PR177 can further comprise, at the 5′ end, a sequence comprising SRE007 and SRE012, or a reverse complement thereof, in a linked double-stranded DNA. In some embodiments, PR176 can further comprise, at the 5′ end, a sequence comprising SRE007 and SRE012, or a reverse complement thereof, in a linked double-stranded DNA. In some embodiments, PR175 can further comprise, at the 5′ end, a sequence comprising SRE007 and SRE012, or a reverse complement thereof, in a linked double-stranded DNA. In some embodiments, PR174 can further comprise, at the 5′ end, a sequence comprising SRE007 and SRE012, or a reverse complement thereof, in a linked double-stranded DNA. In some embodiments, PR173 can further comprise, at the 5′ end, a sequence comprising SRE007 and SRE012, or a reverse complement thereof, in a linked double-stranded DNA. In some embodiments, PR172 can further comprise, at the 5′ end, a sequence comprising SRE007 and SRE012, or a reverse complement thereof, in a linked double-stranded DNA. In some embodiments, PR171 can further comprise, at the 5′ end, a sequence comprising SRE007 and SRE012, or a reverse complement thereof, in a linked double-stranded DNA. In some embodiments, PR170 can further comprise, at the 5′ end, a sequence comprising SRE007 and SRE012, or a reverse complement thereof, in a linked double-stranded DNA. In some embodiments, PR169 can further comprise, at the 5′ end, a sequence comprising SRE007 and SRE012, or a reverse complement thereof, in a linked double-stranded DNA. In some embodiments, PR168 can further comprise, at the 5′ end, a sequence comprising SRE007 and SRE012, or a reverse complement thereof, in a linked double-stranded DNA. In some embodiments, PR167 can further comprise, at the 5′ end, a sequence comprising SRE007 and SRE012, or a reverse complement thereof, in a linked double-stranded DNA. In some embodiments, PR166 can further comprise, at the 5′ end, a sequence comprising SRE007 and SRE012, or a reverse complement thereof, in a linked double-stranded DNA. In some embodiments, PR165 can further comprise, at the 5′ end, a sequence comprising SRE007 and SRE012, or a reverse complement thereof, in a linked double-stranded DNA. In some embodiments, PR159 can further comprise, at the 5′ end, a sequence comprising SRE007 and SRE012, or a reverse complement thereof, in a linked double-stranded DNA. In some embodiments, PR156 can further comprise, at the 5′ end, a sequence comprising SRE007 and SRE012, or a reverse complement thereof, in a linked double-stranded DNA. In some embodiments, PR155 can further comprise, at the 5′ end, a sequence comprising SRE007 and SRE012, or a reverse complement thereof, in a linked double-stranded DNA. In some embodiments, PR154 can further comprise, at the 5′ end, a sequence comprising SRE007 and SRE012, or a reverse complement thereof, in a linked double-stranded DNA. In some embodiments, PR153 can further comprise, at the 5′ end, a sequence comprising SRE007 and SRE012, or a reverse complement thereof, in a linked double-stranded DNA. In some embodiments, PR152 can further comprise, at the 5′ end, a sequence comprising SRE007 and SRE012, or a reverse complement thereof, in a linked double-stranded DNA. In some embodiments, PR151 can further comprise, at the 5′ end, a sequence comprising SRE007 and SRE012, or a reverse complement thereof, in a linked double-stranded DNA. In some embodiments, PR150 can further comprise, at the 5′ end, a sequence comprising SRE007 and SRE012, or a reverse complement thereof, in a linked double-stranded DNA. In some embodiments, PR131 can further comprise, at the 5′ end, a sequence comprising SRE007 and SRE012, or a reverse complement thereof, in a linked double-stranded DNA. In some embodiments, SEQ ID NO: 584 can further comprise, at the 5′ end, a sequence comprising SRE007 and SRE012, or a reverse complement thereof, in a linked double-stranded DNA. In some embodiments, SEQ ID NO: 585 can further comprise, at the 5′ end, a sequence comprising SRE007 and SRE012, or a reverse complement thereof, in a linked double-stranded DNA. In some embodiments, SEQ ID NO: 586 can further comprise, at the 5′ end, a sequence comprising SRE007 and SRE012, or a reverse complement thereof, in a linked double-stranded DNA. In some embodiments, SEQ ID NO: 587 can further comprise, at the 5′ end, a sequence comprising SRE007 and SRE012, or a reverse complement thereof, in a linked double-stranded DNA. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE010 and SRE008, or a reverse complement thereof, in a vector. In some embodiments, PR180 can further comprise, at the 5′ end, a sequence comprising SRE010 and SRE008, or a reverse complement thereof, in a vector. In some embodiments, PR179 can further comprise, at the 5′ end, a sequence comprising SRE010 and SRE008, or a reverse complement thereof, in a vector. In some embodiments, PR178 can further comprise, at the 5′ end, a sequence comprising SRE010 and SRE008, or a reverse complement thereof, in a vector. In some embodiments, PR177 can further comprise, at the 5′ end, a sequence comprising SRE010 and SRE008, or a reverse complement thereof, in a vector. In some embodiments, PR176 can further comprise, at the 5′ end, a sequence comprising SRE010 and SRE008, or a reverse complement thereof, in a vector. In some embodiments, PR175 can further comprise, at the 5′ end, a sequence comprising SRE010 and SRE008, or a reverse complement thereof, in a vector. In some embodiments, PR174 can further comprise, at the 5′ end, a sequence comprising SRE010 and SRE008, or a reverse complement thereof, in a vector. In some embodiments, PR173 can further comprise, at the 5′ end, a sequence comprising SRE010 and SRE008, or a reverse complement thereof, in a vector. In some embodiments, PR172 can further comprise, at the 5′ end, a sequence comprising SRE010 and SRE008, or a reverse complement thereof, in a vector. In some embodiments, PR171 can further comprise, at the 5′ end, a sequence comprising SRE010 and SRE008, or a reverse complement thereof, in a vector. In some embodiments, PR170 can further comprise, at the 5′ end, a sequence comprising SRE010 and SRE008, or a reverse complement thereof, in a vector. In some embodiments, PR169 can further comprise, at the 5′ end, a sequence comprising SRE010 and SRE008, or a reverse complement thereof, in a vector. In some embodiments, PR168 can further comprise, at the 5′ end, a sequence comprising SRE010 and SRE008, or a reverse complement thereof, in a vector. In some embodiments, PR167 can further comprise, at the 5′ end, a sequence comprising SRE010 and SRE008, or a reverse complement thereof, in a vector. In some embodiments, PR166 can further comprise, at the 5′ end, a sequence comprising SRE010 and SRE008, or a reverse complement thereof, in a vector. In some embodiments, PR165 can further comprise, at the 5′ end, a sequence comprising SRE010 and SRE008, or a reverse complement thereof, in a vector. In some embodiments, PR159 can further comprise, at the 5′ end, a sequence comprising SRE010 and SRE008, or a reverse complement thereof, in a vector. In some embodiments, PR156 can further comprise, at the 5′ end, a sequence comprising SRE010 and SRE008, or a reverse complement thereof, in a vector. In some embodiments, PR155 can further comprise, at the 5′ end, a sequence comprising SRE010 and SRE008, or a reverse complement thereof, in a vector. In some embodiments, PR154 can further comprise, at the 5′ end, a sequence comprising SRE010 and SRE008, or a reverse complement thereof, in a vector. In some embodiments, PR153 can further comprise, at the 5′ end, a sequence comprising SRE010 and SRE008, or a reverse complement thereof, in a vector. In some embodiments, PR152 can further comprise, at the 5′ end, a sequence comprising SRE010 and SRE008, or a reverse complement thereof, in a vector. In some embodiments, PR151 can further comprise, at the 5′ end, a sequence comprising SRE010 and SRE008, or a reverse complement thereof, in a vector. In some embodiments, PR150 can further comprise, at the 5′ end, a sequence comprising SRE010 and SRE008, or a reverse complement thereof, in a vector. In some embodiments, PR131 can further comprise, at the 5′ end, a sequence comprising SRE010 and SRE008, or a reverse complement thereof, in a vector. In some embodiments, SEQ ID NO: 584 can further comprise, at the 5′ end, a sequence comprising SRE010 and SRE008, or a reverse complement thereof, in a vector. In some embodiments, SEQ ID NO: 585 can further comprise, at the 5′ end, a sequence comprising SRE010 and SRE008, or a reverse complement thereof, in a vector. In some embodiments, SEQ ID NO: 586 can further comprise, at the 5′ end, a sequence comprising SRE010 and SRE008, or a reverse complement thereof, in a vector. In some embodiments, SEQ ID NO: 587 can further comprise, at the 5′ end, a sequence comprising SRE010 and SRE008, or a reverse complement thereof, in a vector. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE010 and SRE008, or a reverse complement thereof, in a nanoplasmid. In some embodiments, PR180 can further comprise, at the 5′ end, a sequence comprising SRE010 and SRE008, or a reverse complement thereof, in a nanoplasmid. In some embodiments, PR179 can further comprise, at the 5′ end, a sequence comprising SRE010 and SRE008, or a reverse complement thereof, in a nanoplasmid. In some embodiments, PR178 can further comprise, at the 5′ end, a sequence comprising SRE010 and SRE008, or a reverse complement thereof, in a nanoplasmid. In some embodiments, PR177 can further comprise, at the 5′ end, a sequence comprising SRE010 and SRE008, or a reverse complement thereof, in a nanoplasmid. In some embodiments, PR176 can further comprise, at the 5′ end, a sequence comprising SRE010 and SRE008, or a reverse complement thereof, in a nanoplasmid. In some embodiments, PR175 can further comprise, at the 5′ end, a sequence comprising SRE010 and SRE008, or a reverse complement thereof, in a nanoplasmid. In some embodiments, PR174 can further comprise, at the 5′ end, a sequence comprising SRE010 and SRE008, or a reverse complement thereof, in a nanoplasmid. In some embodiments, PR173 can further comprise, at the 5′ end, a sequence comprising SRE010 and SRE008, or a reverse complement thereof, in a nanoplasmid. In some embodiments, PR172 can further comprise, at the 5′ end, a sequence comprising SRE010 and SRE008, or a reverse complement thereof, in a SRE010 and SRE008, or a reverse complement thereof, in a nanoplasmid. In some embodiments, PR170 can further comprise, at the 5′ end, a sequence comprising SRE010 and SRE008, or a reverse complement thereof, in a nanoplasmid. In some embodiments, PR169 can further comprise, at the 5′ end, a sequence comprising SRE010 and SRE008, or a reverse complement thereof, in a nanoplasmid. In some embodiments, PR168 can further comprise, at the 5′ end, a sequence comprising SRE010 and SRE008, or a reverse complement thereof, in a nanoplasmid. In some embodiments, PR167 can further comprise, at the 5′ end, a sequence comprising SRE010 and SRE008, or a reverse complement thereof, in a nanoplasmid. In some embodiments, PR166 can further comprise, at the 5′ end, a sequence comprising SRE010 and SRE008, or a reverse complement thereof, in a nanoplasmid. In some embodiments, PR165 can further comprise, at the 5′ end, a sequence comprising SRE010 and SRE008, or a reverse complement thereof, in a nanoplasmid. In some embodiments, PR159 can further comprise, at the 5′ end, a sequence comprising SRE010 and SRE008, or a reverse complement thereof, in a nanoplasmid. In some embodiments, PR156 can further comprise, at the 5′ end, a sequence comprising SRE010 and SRE008, or a reverse complement thereof, in a nanoplasmid. In some embodiments, PR155 can further comprise, at the 5′ end, a sequence comprising SRE010 and SRE008, or a reverse complement thereof, in a nanoplasmid. In some embodiments, PR154 can further comprise, at the 5′ end, a sequence comprising SRE010 and SRE008, or a reverse complement thereof, in a nanoplasmid. In some embodiments, PR153 can further comprise, at the 5′ end, a sequence comprising SRE010 and SRE008, or a reverse complement thereof, in a nanoplasmid. In some embodiments, PR152 can further comprise, at the 5′ end, a sequence comprising SRE010 and SRE008, or a reverse complement thereof, in a nanoplasmid. In some embodiments, PR151 can further comprise, at the 5′ end, a sequence comprising SRE010 and SRE008, or a reverse complement thereof, in a nanoplasmid. In some embodiments, PR150 can further comprise, at the 5′ end, a sequence comprising SRE010 and SRE008, or a reverse complement thereof, in a nanoplasmid. In some embodiments, PR131 can further comprise, at the 5′ end, a sequence comprising SRE010 and SRE008, or a reverse complement thereof, in a nanoplasmid. In some embodiments, SEQ ID NO: 584 can further comprise, at the 5′ end, a sequence comprising SRE010 and SRE008, or a reverse complement thereof, in a nanoplasmid. In some embodiments, SEQ ID NO: 585 can further comprise, at the 5′ end, a sequence comprising SRE010 and SRE008, or a reverse complement thereof, in a nanoplasmid. In some embodiments, SEQ ID NO: 586 can further comprise, at the 5′ end, a sequence comprising SRE010 and SRE008, or a reverse complement thereof, in a nanoplasmid. In some embodiments, SEQ ID NO: 587 can further comprise, at the 5′ end, a sequence comprising SRE010 and SRE008, or a reverse complement thereof, in a nanoplasmid. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE010 and SRE008, or a reverse complement thereof, in a linked double-stranded DNA. In some embodiments, PR180 can further comprise, at the 5′ end, a sequence comprising SRE010 and SRE008, or a reverse complement thereof, in a linked double-stranded DNA. In some embodiments, PR179 can further comprise, at the 5′ end, a sequence comprising SRE010 and SRE008, or a reverse complement thereof, in a linked double-stranded DNA. In some embodiments, PR178 can further comprise, at the 5′ end, a sequence comprising SRE010 and SRE008, or a reverse complement thereof, in a linked double-stranded DNA. In some embodiments, PR177 can further comprise, at the 5′ end, a sequence comprising SRE010 and SRE008, or a reverse complement thereof, in a linked double-stranded DNA. In some embodiments, PR176 can further comprise, at the 5′ end, a sequence comprising SRE010 and SRE008, or a reverse complement thereof, in a linked double-stranded DNA. In some embodiments, PR175 can further comprise, at the 5′ end, a sequence comprising SRE010 and SRE008, or a reverse complement thereof, in a linked double-stranded DNA. In some embodiments, PR174 can further comprise, at the 5′ end, a sequence comprising SRE010 and SRE008, or a reverse complement thereof, in a linked double-stranded DNA. In some embodiments, PR173 can further comprise, at the 5′ end, a sequence comprising SRE010 and SRE008, or a reverse complement thereof, in a linked double-stranded DNA. In some embodiments, PR172 can further comprise, at the 5′ end, a sequence comprising SRE010 and SRE008, or a reverse complement thereof, in a linked double-stranded DNA. In some embodiments, PR171 can further comprise, at the 5′ end, a sequence comprising SRE010 and SRE008, or a reverse complement thereof, in a linked double-stranded DNA. In some embodiments, PR170 can further comprise, at the 5′ end, a sequence comprising SRE010 and SRE008, or a reverse complement thereof, in a linked double-stranded DNA. In some embodiments, PR169 can further comprise, at the 5′ end, a sequence comprising SRE010 and SRE008, or a reverse complement thereof, in a linked double-stranded DNA. In some embodiments, PR168 can further comprise, at the 5′ end, a sequence comprising SRE010 and SRE008, or a reverse complement thereof, in a linked double-stranded DNA. In some embodiments, PR167 can further comprise, at the 5′ end, a sequence comprising SRE010 and SRE008, or a reverse complement thereof, in a linked double-stranded DNA. In some embodiments, PR166 can further comprise, at the 5′ end, a sequence comprising SRE010 and SRE008, or a reverse complement thereof, in a linked double-stranded DNA. In some embodiments, PR165 can further comprise, at the 5′ end, a sequence comprising SRE010 and SRE008, or a reverse complement thereof, in a linked double-stranded DNA. In some embodiments, PR159 can further comprise, at the 5′ end, a sequence comprising SRE010 and SRE008, or a reverse complement thereof, in a linked double-stranded DNA. In some embodiments, PR156 can further comprise, at the 5′ end, a sequence comprising SRE010 and SRE008, or a reverse complement thereof, in a linked double-stranded DNA. In some embodiments, PR155 can further comprise, at the 5′ end, a sequence comprising SRE010 and SRE008, or a reverse complement thereof, in a linked double-stranded DNA. In some embodiments, PR154 can further comprise, at the 5′ end, a sequence comprising SRE010 and SRE008, or a reverse complement thereof, in a linked double-stranded DNA. In some embodiments, PR153 can further comprise, at the 5′ end, a sequence comprising SRE010 and SRE008, or a reverse complement thereof, in a linked double-stranded DNA. In some embodiments, PR152 can further comprise, at the 5′ end, a sequence comprising SRE010 and SRE008, or a reverse complement thereof, in a linked double-stranded DNA. In some embodiments, PR151 can further comprise, at the 5′ end, a sequence comprising SRE010 and SRE008, or a reverse complement thereof, in a linked double-stranded DNA. In some embodiments, PR150 can further comprise, at the 5′ end, a sequence comprising SRE010 and SRE008, or a reverse complement thereof, in a linked double-stranded DNA. In some embodiments, PR131 can further comprise, at the 5′ end, a sequence comprising SRE010 and SRE008, or a reverse complement thereof, in a linked double-stranded DNA. In some embodiments, SEQ ID NO: 584 can further comprise, at the 5′ end, a sequence comprising SRE010 and SRE008, or a reverse complement thereof, in a linked double-stranded DNA. In some embodiments, SEQ ID NO: 585 can further comprise, at the 5′ end, a sequence comprising SRE010 and SRE008, or a reverse complement thereof, in a linked double-stranded DNA. In some embodiments, SEQ ID NO: 586 can further comprise, at the 5′ end, a sequence comprising SRE010 and SRE008, or a reverse complement thereof, in a linked double-stranded DNA. In some embodiments, SEQ ID NO: 587 can further comprise, at the 5′ end, a sequence comprising SRE010 and SRE008, or a reverse complement thereof, in a linked double-stranded DNA. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE012 and SRE008, or a reverse complement thereof, in a vector. In some embodiments, PR180 can further comprise, at the 5′ end, a sequence comprising SRE012 and SRE008, or a reverse complement thereof, in a vector. In some embodiments, PR179 can further comprise, at the 5′ end, a sequence comprising SRE012 and SRE008, or a reverse complement thereof, in a vector. In some embodiments, PR178 can further comprise, at the 5′ end, a sequence comprising SRE012 and SRE008, or a reverse complement thereof, in a vector. In some embodiments, PR177 can further comprise, at the 5′ end, a sequence comprising SRE012 and SRE008, or a reverse complement thereof, in a vector. In some embodiments, PR176 can further comprise, at the 5′ end, a sequence comprising SRE012 and SRE008, or a reverse complement thereof, in a vector. In some embodiments, PR175 can further comprise, at the 5′ end, a sequence comprising SRE012 and SRE008, or a reverse complement thereof, in a vector. In some embodiments, PR174 can further comprise, at the 5′ end, a sequence comprising SRE012 and SRE008, or a reverse complement thereof, in a vector. In some embodiments, PR173 can further comprise, at the 5′ end, a sequence comprising SRE012 and SRE008, or a reverse complement thereof, in a vector. In some embodiments, PR172 can further comprise, at the 5′ end, a sequence comprising SRE012 and SRE008, or a reverse complement thereof, in a vector. In some embodiments, PR171 can further comprise, at the 5′ end, a sequence comprising SRE012 and SRE008, or a reverse complement thereof, in a vector. In some embodiments, PR170 can further comprise, at the 5′ end, a sequence comprising SRE012 and SRE008, or a reverse complement thereof, in a vector. In some embodiments, PR169 can further comprise, at the 5′ end, a sequence comprising SRE012 and SRE008, or a reverse complement thereof, in a vector. In some embodiments, PR168 can further comprise, at the 5′ end, a sequence comprising SRE012 and SRE008, or a reverse complement thereof, in a vector. In some embodiments, PR167 can further comprise, at the 5′ end, a sequence comprising SRE012 and SRE008, or a reverse complement thereof, in a vector. In some embodiments, PR166 can further comprise, at the 5′ end, a sequence comprising SRE012 and SRE008, or a reverse complement thereof, in a vector. In some embodiments, PR165 can further comprise, at the 5′ end, a sequence comprising SRE012 and SRE008, or a reverse complement thereof, in a vector. In some embodiments, PR159 can further comprise, at the 5′ end, a sequence comprising SRE012 and SRE008, or a reverse complement thereof, in a vector. In some embodiments, PR156 can further comprise, at the 5′ end, a sequence comprising SRE012 and SRE008, or a reverse complement thereof, in a vector. In some embodiments, PR155 can further comprise, at the 5′ end, a sequence comprising SRE012 and SRE008, or a reverse complement thereof, in a vector. In some embodiments, PR154 can further comprise, at the 5′ end, a sequence comprising SRE012 and SRE008, or a reverse complement thereof, in a vector. In some embodiments, PR153 can further comprise, at the 5′ end, a sequence comprising SRE012 and SRE008, or a reverse complement thereof, in a vector. In some embodiments, PR152 can further comprise, at the 5′ end, a sequence comprising SRE012 and SRE008, or a reverse complement thereof, in a vector. In some embodiments, PR151 can further comprise, at the 5′ end, a sequence comprising SRE012 and SRE008, or a reverse complement thereof, in a vector. In some embodiments, PR150 can further comprise, at the 5′ end, a sequence comprising SRE012 and SRE008, or a reverse complement thereof, in a vector. In some embodiments, PR131 can further comprise, at the 5′ end, a sequence comprising SRE012 and SRE008, or a reverse complement thereof, in a vector. In some embodiments, SEQ ID NO: 584 can further comprise, at the 5′ end, a sequence comprising SRE012 and SRE008, or a reverse complement thereof, in a vector. In some embodiments, SEQ ID NO: 585 can further comprise, at the 5′ end, a sequence comprising SRE012 and SRE008, or a reverse complement thereof, in a vector. In some embodiments, SEQ ID NO: 586 can further comprise, at the 5′ end, a sequence comprising SRE012 and SRE008, or a reverse complement thereof, in a vector. In some embodiments, SEQ ID NO: 587 can further comprise, at the 5′ end, a sequence comprising SRE012 and SRE008, or a reverse complement thereof, in a vector. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE012 and SRE008, or a reverse complement thereof, in a nanoplasmid. In some embodiments, PR180 can further comprise, at the 5′ end, a sequence comprising SRE012 and SRE008, or a reverse complement thereof, in a nanoplasmid. In some embodiments, PR179 can further comprise, at the 5′ end, a sequence comprising SRE012 and SRE008, or a reverse complement thereof, in a nanoplasmid. In some embodiments, PR178 can further comprise, at the 5′ end, a sequence comprising SRE012 and SRE008, or a reverse complement thereof, in a nanoplasmid. In some embodiments, PR177 can further comprise, at the 5′ end, a sequence comprising SRE012 and SRE008, or a reverse complement thereof, in a nanoplasmid. In some embodiments, PR176 can further comprise, at the 5′ end, a sequence comprising SRE012 and SRE008, or a reverse complement thereof, in a nanoplasmid. In some embodiments, PR175 can further comprise, at the 5′ end, a sequence comprising SRE012 and SRE008, or a reverse complement thereof, in a nanoplasmid. In some embodiments, PR174 can further comprise, at the 5′ end, a sequence comprising SRE012 and SRE008, or a reverse complement thereof, in a nanoplasmid. In some embodiments, PR173 can further comprise, at the 5′ end, a sequence comprising SRE012 and SRE008, or a reverse complement thereof, in a nanoplasmid. In some embodiments, PR172 can further comprise, at the 5′ end, a sequence comprising SRE012 and SRE008, or a reverse complement thereof, in a nanoplasmid. In some embodiments, PR171 can further comprise, at the 5′ end, a sequence comprising SRE012 and SRE008, or a reverse complement thereof, in a nanoplasmid. In some embodiments, PR170 can further comprise, at the 5′ end, a sequence comprising SRE012 and SRE008, or a reverse complement thereof, in a nanoplasmid. In some embodiments, PR169 can further comprise, at the 5′ end, a sequence comprising SRE012 and SRE008, or a reverse complement thereof, in a nanoplasmid. In some embodiments, PR168 can further comprise, at the 5′ end, a sequence comprising SRE012 and SRE008, or a reverse complement thereof, in a nanoplasmid. In some embodiments, PR167 can further comprise, at the 5′ end, a sequence comprising SRE012 and SRE008, or a reverse complement thereof, in a nanoplasmid. In some embodiments, PR166 can further comprise, at the 5′ end, a sequence comprising SRE012 and SRE008, or a reverse complement thereof, in a nanoplasmid. In some embodiments, PR165 can further comprise, at the 5′ end, a sequence comprising SRE012 and SRE008, or a reverse complement thereof, in a nanoplasmid. In some embodiments, PR159 can further comprise, at the 5′ end, a sequence comprising SRE012 and SRE008, or a reverse complement thereof, in a nanoplasmid. In some embodiments, PR156 can further comprise, at the 5′ end, a sequence comprising SRE012 and SRE008, or a reverse complement thereof, in a nanoplasmid. In some embodiments, PR155 can further comprise, at the 5′ end, a sequence comprising SRE012 and SRE008, or a reverse complement thereof, in a nanoplasmid. In some embodiments, PR154 can further comprise, at the 5′ end, a sequence comprising SRE012 and SRE008, or a reverse complement thereof, in a nanoplasmid. In some embodiments, PR153 can further comprise, at the 5′ end, a sequence comprising SRE012 and SRE008, or a reverse complement thereof, in a nanoplasmid. In some embodiments, PR152 can further comprise, at the 5′ end, a sequence comprising SRE012 and SRE008, or a reverse complement thereof, in a nanoplasmid. In some embodiments, PR151 can further comprise, at the 5′ end, a sequence comprising SRE012 and SRE008, or a reverse complement thereof, in a nanoplasmid. In some embodiments, PR150 can further comprise, at the 5′ end, a sequence comprising SRE012 and SRE008, or a reverse complement thereof, in a nanoplasmid. In some embodiments, PR131 can further comprise, at the 5′ end, a sequence comprising SRE012 and SRE008, or a reverse complement thereof, in a nanoplasmid. In some embodiments, SEQ ID NO: 584 can further comprise, at the 5′ end, a sequence comprising SRE012 and SRE008, or a reverse complement thereof, in a nanoplasmid. In some embodiments, SEQ ID NO: 585 can further comprise, at the 5′ end, a sequence comprising SRE012 and SRE008, or a reverse complement thereof, in a nanoplasmid. In some embodiments, SEQ ID NO: 586 can further comprise, at the 5′ end, a sequence comprising SRE012 and SRE008, or a reverse complement thereof, in a nanoplasmid. In some embodiments, SEQ ID NO: 587 can further comprise, at the 5′ end, a sequence comprising SRE012 and SRE008, or a reverse complement thereof, in a nanoplasmid. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE012 and SRE008, or a reverse complement thereof, in a linked double-stranded DNA. In some embodiments, PR180 can further comprise, at the 5′ end, a sequence comprising SRE012 and SRE008, or a reverse complement thereof, in a linked double-stranded DNA. In some embodiments, PR179 can further comprise, at the 5′ end, a sequence comprising SRE012 and SRE008, or a reverse complement thereof, in a linked double-stranded DNA. In some embodiments, PR178 can further comprise, at the 5′ end, a sequence comprising SRE012 and SRE008, or a reverse complement thereof, in a linked double-stranded DNA. In some embodiments, PR177 can further comprise, at the 5′ end, a sequence comprising SRE012 and SRE008, or a reverse complement thereof, in a linked double-stranded DNA. In some embodiments, PR176 can further comprise, at the 5′ end, a sequence comprising SRE012 and SRE008, or a reverse complement thereof, in a linked double-stranded DNA. In some embodiments, PR175 can further comprise, at the 5′ end, a sequence comprising SRE012 and SRE008, or a reverse complement thereof, in a linked double-stranded DNA. In some embodiments, PR174 can further comprise, at the 5′ end, a sequence comprising SRE012 and SRE008, or a reverse complement thereof, in a linked double-stranded DNA. In some embodiments, PR173 can further comprise, at the 5′ end, a sequence comprising SRE012 and SRE008, or a reverse complement thereof, in a linked double-stranded DNA. In some embodiments, PR172 can further comprise, at the 5′ end, a sequence comprising SRE012 and SRE008, or a reverse complement thereof, in a linked double-stranded DNA. In some embodiments, PR171 can further comprise, at the 5′ end, a sequence comprising SRE012 and SRE008, or a reverse complement thereof, in a linked double-stranded DNA. In some embodiments, PR170 can further comprise, at the 5′ end, a sequence comprising SRE012 and SRE008, or a reverse complement thereof, in a linked double-stranded DNA. In some embodiments, PR169 can further comprise, at the 5′ end, a sequence comprising SRE012 and SRE008, or a reverse complement thereof, in a linked double-stranded DNA. In some embodiments, PR168 can further comprise, at the 5′ end, a sequence comprising SRE012 and SRE008, or a reverse complement thereof, in a linked double-stranded DNA. In some embodiments, PR167 can further comprise, at the 5′ end, a sequence comprising SRE012 and SRE008, or a reverse complement thereof, in a linked double-stranded DNA. In some embodiments, PR166 can further comprise, at the 5′ end, a sequence comprising SRE012 and SRE008, or a reverse complement thereof, in a linked double-stranded DNA. In some embodiments, PR165 can further comprise, at the 5′ end, a sequence comprising SRE012 and SRE008, or a reverse complement thereof, in a linked double-stranded DNA. In some embodiments, PR159 can further comprise, at the 5′ end, a sequence comprising SRE012 and SRE008, or a reverse complement thereof, in a linked double-stranded DNA. In some embodiments, PR156 can further comprise, at the 5′ end, a sequence comprising SRE012 and SRE008, or a reverse complement thereof, in a linked double-stranded DNA. In some embodiments, PR155 can further comprise, at the 5′ end, a sequence comprising SRE012 and SRE008, or a reverse complement thereof, in a linked double-stranded DNA. In some embodiments, PR154 can further comprise, at the 5′ end, a sequence comprising SRE012 and SRE008, or a reverse complement thereof, in a linked double-stranded DNA. In some embodiments, PR153 can further comprise, at the 5′ end, a sequence comprising SRE012 and SRE008, or a reverse complement thereof, in a linked double-stranded DNA. In some embodiments, PR152 can further comprise, at the 5′ end, a sequence comprising SRE012 and SRE008, or a reverse complement thereof, in a linked double-stranded DNA. In some embodiments, PR151 can further comprise, at the 5′ end, a sequence comprising SRE012 and SRE008, or a reverse complement thereof, in a linked double-stranded DNA. In some embodiments, PR150 can further comprise, at the 5′ end, a sequence comprising SRE012 and SRE008, or a reverse complement thereof, in a linked double-stranded DNA. In some embodiments, PR131 can further comprise, at the 5′ end, a sequence comprising SRE012 and SRE008, or a reverse complement thereof, in a linked double-stranded DNA. In some embodiments, SEQ ID NO: 584 can further comprise, at the 5′ end, a sequence comprising SRE012 and SRE008, or a reverse complement thereof, in a linked double-stranded DNA. In some embodiments, SEQ ID NO: 585 can further comprise, at the 5′ end, a sequence comprising SRE012 and SRE008, or a reverse complement thereof, in a linked double-stranded DNA. In some embodiments, SEQ ID NO: 586 can further comprise, at the 5′ end, a sequence comprising SRE012 and SRE008, or a reverse complement thereof, in a linked double-stranded DNA. In some embodiments, SEQ ID NO: 587 can further comprise, at the 5′ end, a sequence comprising SRE012 and SRE008, or a reverse complement thereof, in a linked double-stranded DNA. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE012 and SRE010, or a reverse complement thereof, in a vector. In some embodiments, PR180 can further comprise, at the 5′ end, a sequence comprising SRE012 and SRE010, or a reverse complement thereof, in a vector. In some embodiments, PR179 can further comprise, at the 5′ end, a sequence comprising SRE012 and SRE010, or a reverse complement thereof, in a vector. In some embodiments, PR178 can further comprise, at the 5′ end, a sequence comprising SRE012 and SRE010, or a reverse complement thereof, in a vector. In some embodiments, PR177 can further comprise, at the 5′ end, a sequence comprising SRE012 and SRE010, or a reverse complement thereof, in a vector. In some embodiments, PR176 can further comprise, at the 5′ end, a sequence comprising SRE012 and SRE010, or a reverse complement thereof, in a vector. In some embodiments, PR175 can further comprise, at the 5′ end, a sequence comprising SRE012 and SRE010, or a reverse complement thereof, in a vector. In some embodiments, PR174 can further comprise, at the 5′ end, a sequence comprising SRE012 and SRE010, or a reverse complement thereof, in a vector. In some embodiments, PR173 can further comprise, at the 5′ end, a sequence comprising SRE012 and SRE010, or a reverse complement thereof, in a vector. In some embodiments, PR172 can further comprise, at the 5′ end, a sequence comprising SRE012 and SRE010, or a reverse complement thereof, in a vector. In some embodiments, PR171 can further comprise, at the 5′ end, a sequence comprising SRE012 and SRE010, or a reverse complement thereof, in a vector. In some embodiments, PR170 can further comprise, at the 5′ end, a sequence comprising SRE012 and SRE010, or a reverse complement thereof, in a vector. In some embodiments, PR169 can further comprise, at the 5′ end, a sequence comprising SRE012 and SRE010, or a reverse complement thereof, in a vector. In some embodiments, PR168 can further comprise, at the 5′ end, a sequence comprising SRE012 and SRE010, or a reverse complement thereof, in a vector. In some embodiments, PR167 can further comprise, at the 5′ end, a sequence comprising SRE012 and SRE010, or a reverse complement thereof, in a vector. In some embodiments, PR166 can further comprise, at the 5′ end, a sequence comprising SRE012 and SRE010, or a reverse complement thereof, in a vector. In some embodiments, PR165 can further comprise, at the 5′ end, a sequence comprising SRE012 and SRE010, or a reverse complement thereof, in a vector. In some embodiments, PR159 can further comprise, at the 5′ end, a sequence comprising SRE012 and SRE010, or a reverse complement thereof, in a vector. In some embodiments, PR156 can further comprise, at the 5′ end, a sequence comprising SRE012 and SRE010, or a reverse complement thereof, in a vector. In some embodiments, PR155 can further comprise, at the 5′ end, a sequence comprising SRE012 and SRE010, or a reverse complement thereof, in a vector. In some embodiments, PR154 can further comprise, at the 5′ end, a sequence comprising SRE012 and SRE010, or a reverse complement thereof, in a vector. In some embodiments, PR153 can further comprise, at the 5′ end, a sequence comprising SRE012 and SRE010, or a reverse complement thereof, in a vector. In some embodiments, PR152 can further comprise, at the 5′ end, a sequence comprising SRE012 and SRE010, or a reverse complement thereof, in a vector. In some embodiments, PR151 can further comprise, at the 5′ end, a sequence comprising SRE012 and SRE010, or a reverse complement thereof, in a vector. In some embodiments, PR150 can further comprise, at the 5′ end, a sequence comprising SRE012 and SRE010, or a reverse complement thereof, in a vector. In some embodiments, PR131 can further comprise, at the 5′ end, a sequence comprising SRE012 and SRE010, or a reverse complement thereof, in a vector. In some embodiments, SEQ ID NO: 584 can further comprise, at the 5′ end, a sequence comprising SRE012 and SRE010, or a reverse complement thereof, in a vector. In some embodiments, SEQ ID NO: 585 can further comprise, at the 5′ end, a sequence comprising SRE012 and SRE010, or a reverse complement thereof, in a vector. In some embodiments, SEQ ID NO: 586 can further comprise, at the 5′ end, a sequence comprising SRE012 and SRE010, or a reverse complement thereof, in a vector. In some embodiments, SEQ ID NO: 587 can further comprise, at the 5′ end, a sequence comprising SRE012 and SRE010, or a reverse complement thereof, in a vector. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE012 and SRE010, or a reverse complement thereof, in a nanoplasmid. In some embodiments, PR180 can further comprise, at the 5′ end, a sequence comprising SRE012 and SRE010, or a reverse complement thereof, in a nanoplasmid. In some embodiments, PR179 can further comprise, at the 5′ end, a sequence comprising SRE012 and SRE010, or a reverse complement thereof, in a nanoplasmid. In some embodiments, PR178 can further comprise, at the 5′ end, a sequence comprising SRE012 and SRE010, or a reverse complement thereof, in a nanoplasmid. In some embodiments, PR177 can further comprise, at the 5′ end, a sequence comprising SRE012 and SRE010, or a reverse complement thereof, in a nanoplasmid. In some embodiments, PR176 can further comprise, at the 5′ end, a sequence comprising SRE012 and SRE010, or a reverse complement thereof, in a nanoplasmid. In some embodiments, PR175 can further comprise, at the 5′ end, a sequence comprising SRE012 and SRE010, or a reverse complement thereof, in a nanoplasmid. In some embodiments, PR174 can further comprise, at the 5′ end, a sequence comprising SRE012 and SRE010, or a reverse complement thereof, in a nanoplasmid. In some embodiments, PR173 can further comprise, at the 5′ end, a sequence comprising SRE012 and SRE010, or a reverse complement thereof, in a nanoplasmid. In some embodiments, PR172 can further comprise, at the 5′ end, a sequence comprising SRE012 and SRE010, or a reverse complement thereof, in a SRE012 and SRE010, or a reverse complement thereof, in a nanoplasmid. In some embodiments, PR170 can further comprise, at the 5′ end, a sequence comprising SRE012 and SRE010, or a reverse complement thereof, in a nanoplasmid. In some embodiments, PR169 can further comprise, at the 5′ end, a sequence comprising SRE012 and SRE010, or a reverse complement thereof, in a nanoplasmid. In some embodiments, PR168 can further comprise, at the 5′ end, a sequence comprising SRE012 and SRE010, or a reverse complement thereof, in a nanoplasmid. In some embodiments, PR167 can further comprise, at the 5′ end, a sequence comprising SRE012 and SRE010, or a reverse complement thereof, in a nanoplasmid. In some embodiments, PR166 can further comprise, at the 5′ end, a sequence comprising SRE012 and SRE010, or a reverse complement thereof, in a nanoplasmid. In some embodiments, PR165 can further comprise, at the 5′ end, a sequence comprising SRE012 and SRE010, or a reverse complement thereof, in a nanoplasmid. In some embodiments, PR159 can further comprise, at the 5′ end, a sequence comprising SRE012 and SRE010, or a reverse complement thereof, in a nanoplasmid. In some embodiments, PR156 can further comprise, at the 5′ end, a sequence comprising SRE012 and SRE010, or a reverse complement thereof, in a nanoplasmid. In some embodiments, PR155 can further comprise, at the 5′ end, a sequence comprising SRE012 and SRE010, or a reverse complement thereof, in a nanoplasmid. In some embodiments, PR154 can further comprise, at the 5′ end, a sequence comprising SRE012 and SRE010, or a reverse complement thereof, in a nanoplasmid. In some embodiments, PR153 can further comprise, at the 5′ end, a sequence comprising SRE012 and SRE010, or a reverse complement thereof, in a nanoplasmid. In some embodiments, PR152 can further comprise, at the 5′ end, a sequence comprising SRE012 and SRE010, or a reverse complement thereof, in a nanoplasmid. In some embodiments, PR151 can further comprise, at the 5′ end, a sequence comprising SRE012 and SRE010, or a reverse complement thereof, in a nanoplasmid. In some embodiments, PR150 can further comprise, at the 5′ end, a sequence comprising SRE012 and SRE010, or a reverse complement thereof, in a nanoplasmid. In some embodiments, PR131 can further comprise, at the 5′ end, a sequence comprising SRE012 and SRE010, or a reverse complement thereof, in a nanoplasmid. In some embodiments, SEQ ID NO: 584 can further comprise, at the 5′ end, a sequence comprising SRE012 and SRE010, or a reverse complement thereof, in a nanoplasmid. In some embodiments, SEQ ID NO: 585 can further comprise, at the 5′ end, a sequence comprising SRE012 and SRE010, or a reverse complement thereof, in a nanoplasmid. In some embodiments, SEQ ID NO: 586 can further comprise, at the 5′ end, a sequence comprising SRE012 and SRE010, or a reverse complement thereof, in a nanoplasmid. In some embodiments, SEQ ID NO: 587 can further comprise, at the 5′ end, a sequence comprising SRE012 and SRE010, or a reverse complement thereof, in a nanoplasmid. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE012 and SRE010, or a reverse complement thereof, in a linked double-stranded DNA. In some embodiments, PR180 can further comprise, at the 5′ end, a sequence comprising SRE012 and SRE010, or a reverse complement thereof, in a linked double-stranded DNA. In some embodiments, PR179 can further comprise, at the 5′ end, a sequence comprising SRE012 and SRE010, or a reverse complement thereof, in a linked double-stranded DNA. In some embodiments, PR178 can further comprise, at the 5′ end, a sequence comprising SRE012 and SRE010, or a reverse complement thereof, in a linked double-stranded DNA. In some embodiments, PR177 can further comprise, at the 5′ end, a sequence comprising SRE012 and SRE010, or a reverse complement thereof, in a linked double-stranded DNA. In some embodiments, PR176 can further comprise, at the 5′ end, a sequence comprising SRE012 and SRE010, or a reverse complement thereof, in a linked double-stranded DNA. In some embodiments, PR175 can further comprise, at the 5′ end, a sequence comprising SRE012 and SRE010, or a reverse complement thereof, in a linked double-stranded DNA. In some embodiments, PR174 can further comprise, at the 5′ end, a sequence comprising SRE012 and SRE010, or a reverse complement thereof, in a linked double-stranded DNA. In some embodiments, PR173 can further comprise, at the 5′ end, a sequence comprising SRE012 and SRE010, or a reverse complement thereof, in a linked double-stranded DNA. In some embodiments, PR172 can further comprise, at the 5′ end, a sequence comprising SRE012 and SRE010, or a reverse complement thereof, in a linked double-stranded DNA. In some embodiments, PR171 can further comprise, at the 5′ end, a sequence comprising SRE012 and SRE010, or a reverse complement thereof, in a linked double-stranded DNA. In some embodiments, PR170 can further comprise, at the 5′ end, a sequence comprising SRE012 and SRE010, or a reverse complement thereof, in a linked double-stranded DNA. In some embodiments, PR169 can further comprise, at the 5′ end, a sequence comprising SRE012 and SRE010, or a reverse complement thereof, in a linked double-stranded DNA. In some embodiments, PR168 can further comprise, at the 5′ end, a sequence comprising SRE012 and SRE010, or a reverse complement thereof, in a linked double-stranded DNA. In some embodiments, PR167 can further comprise, at the 5′ end, a sequence comprising SRE012 and SRE010, or a reverse complement thereof, in a linked double-stranded DNA. In some embodiments, PR166 can further comprise, at the 5′ end, a sequence comprising SRE012 and SRE010, or a reverse complement thereof, in a linked double-stranded DNA. In some embodiments, PR165 can further comprise, at the 5′ end, a sequence comprising SRE012 and SRE010, or a reverse complement thereof, in a linked double-stranded DNA. In some embodiments, PR159 can further comprise, at the 5′ end, a sequence comprising SRE012 and SRE010, or a reverse complement thereof, in a linked double-stranded DNA. In some embodiments, PR156 can further comprise, at the 5′ end, a sequence comprising SRE012 and SRE010, or a reverse complement thereof, in a linked double-stranded DNA. In some embodiments, PR155 can further comprise, at the 5′ end, a sequence comprising SRE012 and SRE010, or a reverse complement thereof, in a linked double-stranded DNA. In some embodiments, PR154 can further comprise, at the 5′ end, a sequence comprising SRE012 and SRE010, or a reverse complement thereof, in a linked double-stranded DNA. In some embodiments, PR153 can further comprise, at the 5′ end, a sequence comprising SRE012 and SRE010, or a reverse complement thereof, in a linked double-stranded DNA. In some embodiments, PR152 can further comprise, at the 5′ end, a sequence comprising SRE012 and SRE010, or a reverse complement thereof, in a linked double-stranded DNA. In some embodiments, PR151 can further comprise, at the 5′ end, a sequence comprising SRE012 and SRE010, or a reverse complement thereof, in a linked double-stranded DNA. In some embodiments, PR150 can further comprise, at the 5′ end, a sequence comprising SRE012 and SRE010, or a reverse complement thereof, in a linked double-stranded DNA. In some embodiments, PR131 can further comprise, at the 5′ end, a sequence comprising SRE012 and SRE010, or a reverse complement thereof, in a linked double-stranded DNA. In some embodiments, SEQ ID NO: 584 can further comprise, at the 5′ end, a sequence comprising SRE012 and SRE010, or a reverse complement thereof, in a linked double-stranded DNA. In some embodiments, SEQ ID NO: 585 can further comprise, at the 5′ end, a sequence comprising SRE012 and SRE010, or a reverse complement thereof, in a linked double-stranded DNA. In some embodiments, SEQ ID NO: 586 can further comprise, at the 5′ end, a sequence comprising SRE012 and SRE010, or a reverse complement thereof, in a linked double-stranded DNA. In some embodiments, SEQ ID NO: 587 can further comprise, at the 5′ end, a sequence comprising SRE012 and SRE010, or a reverse complement thereof, in a linked double-stranded DNA. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE007, or a reverse complement thereof, in a vector. In some embodiments, PR180 can further comprise, at the 5′ end, a sequence comprising SRE007, or a reverse complement thereof, in a vector. In some embodiments, PR179 can further comprise, at the 5′ end, a sequence comprising SRE007, or a reverse complement thereof, in a vector. In some embodiments, PR178 can further comprise, at the 5′ end, a sequence comprising SRE007, or a reverse complement thereof, in a vector. In some embodiments, PR177 can further comprise, at the 5′ end, a sequence comprising SRE007, or a reverse complement thereof, in a vector. In some embodiments, PR176 can further comprise, at the 5′ end, a sequence comprising SRE007, or a reverse complement thereof, in a vector. In some embodiments, PR175 can further comprise, at the 5′ end, a sequence comprising SRE007, or a reverse complement thereof, in a vector. In some embodiments, PR174 can further comprise, at the 5′ end, a sequence comprising SRE007, or a reverse complement thereof, in a vector. In some embodiments, PR173 can further comprise, at the 5′ end, a sequence comprising SRE007, or a reverse complement thereof, in a vector. In some embodiments, PR172 can further comprise, at the 5′ end, a sequence comprising SRE007, or a reverse complement thereof, in a vector. In some embodiments, PR171 can further comprise, at the 5′ end, a sequence comprising SRE007, or a reverse complement thereof, in a vector. In some embodiments, PR170 can further comprise, at the 5′ end, a sequence comprising SRE007, or a reverse complement thereof, in a vector. In some embodiments, PR169 can further comprise, at the 5′ end, a sequence comprising SRE007, or a reverse complement thereof, in a vector. In some embodiments, PR168 can further comprise, at the 5′ end, a sequence comprising SRE007, or a reverse complement thereof, in a vector. In some embodiments, PR167 can further comprise, at the 5′ end, a sequence comprising SRE007, or a reverse complement thereof, in a vector. In some embodiments, PR166 can further comprise, at the 5′ end, a sequence comprising SRE007, or a reverse complement thereof, in a vector. In some embodiments, PR165 can further comprise, at the 5′ end, a sequence comprising SRE007, or a reverse complement thereof, in a vector. In some embodiments, PR159 can further comprise, at the 5′ end, a sequence comprising SRE007, or a reverse complement thereof, in a vector. In some embodiments, PR156 can further comprise, at the 5′ end, a sequence comprising SRE007 and SRE010, or a reverse complement thereof, in a vector. In some embodiments, PR155 can further comprise, at the 5′ end, a sequence comprising SRE007, or a reverse complement thereof, in a vector. In some embodiments, PR154 can further comprise, at the 5′ end, a sequence comprising SRE007, or a reverse complement thereof, in a vector. In some embodiments, PR153 can further comprise, at the 5′ end, a sequence comprising SRE007, or a reverse complement thereof, in a vector. In some embodiments, PR152 can further comprise, at the 5′ end, a sequence comprising SRE007, or a reverse complement thereof, in a vector. In some embodiments, PR151 can further comprise, at the 5′ end, a sequence comprising SRE007, or a reverse complement thereof, in a vector. In some embodiments, PR150 can further comprise, at the 5′ end, a sequence comprising SRE007, or a reverse complement thereof, in a vector. In some embodiments, PR131 can further comprise, at the 5′ end, a sequence comprising SRE007, or a reverse complement thereof, in a vector. In some embodiments, SEQ ID NO: 584 can further comprise, at the 5′ end, a sequence comprising SRE007, or a reverse complement thereof, in a vector. In some embodiments, SEQ ID NO: 585 can further comprise, at the 5′ end, a sequence comprising SRE007, or a reverse complement thereof, in a vector. In some embodiments, SEQ ID NO: 586 can further comprise, at the 5′ end, a sequence comprising SRE007, or a reverse complement thereof, in a vector. In some embodiments, SEQ ID NO: 587 can further comprise, at the 5′ end, a sequence comprising SRE007, or a reverse complement thereof, in a vector. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE007, or a reverse complement thereof, in a nanoplasmid. In some embodiments, PR180 can further comprise, at the 5′ end, a sequence comprising SRE007, or a reverse complement thereof, in a nanoplasmid. In some embodiments, PR179 can further comprise, at the 5′ end, a sequence comprising SRE007, or a reverse complement thereof, in a nanoplasmid. In some embodiments, PR178 can further comprise, at the 5′ end, a sequence comprising SRE007, or a reverse complement thereof, in a nanoplasmid. In some embodiments, PR177 can further comprise, at the 5′ end, a sequence comprising SRE007, or a reverse complement thereof, in a nanoplasmid. In some embodiments, PR176 can further comprise, at the 5′ end, a sequence comprising SRE007, or a reverse complement thereof, in a nanoplasmid. In some embodiments, PR175 can further comprise, at the 5′ end, a sequence comprising SRE007, or a reverse complement thereof, in a nanoplasmid. In some embodiments, PR174 can further comprise, at the 5′ end, a sequence comprising SRE007, or a reverse complement thereof, in a nanoplasmid. In some embodiments, PR173 can further comprise, at the 5′ end, a sequence comprising SRE007, or a reverse complement thereof, in a nanoplasmid. In some embodiments, PR172 can further comprise, at the 5′ end, a sequence comprising SRE007, or a reverse complement thereof, in a nanoplasmid. In some embodiments, PR171 can further comprise, at the 5′ end, a sequence comprising SRE007, or a reverse complement thereof, in a nanoplasmid. In some embodiments, PR170 can further comprise, at the 5′ end, a sequence comprising SRE007, or a reverse complement thereof, in a nanoplasmid. In some embodiments, PR169 can further comprise, at the 5′ end, a sequence comprising SRE007, or a reverse complement thereof, in a nanoplasmid. In some embodiments, PR168 can further comprise, at the 5′ end, a sequence comprising SRE007, or a reverse complement thereof, in a nanoplasmid. In some embodiments, PR167 can further comprise, at the 5′ end, a sequence comprising SRE007, or a reverse complement thereof, in a nanoplasmid. In some embodiments, PR166 can further comprise, at the 5′ end, a sequence comprising SRE007, or a reverse complement thereof, in a nanoplasmid. In some embodiments, PR165 can further comprise, at the 5′ end, a sequence comprising SRE007, or a reverse complement thereof, in a nanoplasmid. In some embodiments, PR159 can further comprise, at the 5′ end, a sequence comprising SRE007, or a reverse complement thereof, in a nanoplasmid. In some embodiments, PR156 can further comprise, at the 5′ end, a sequence comprising SRE007 and SRE010, or a reverse complement thereof, in a nanoplasmid. In some embodiments, PR155 can further comprise, at the 5′ end, a sequence comprising SRE007, or a reverse complement thereof, in a nanoplasmid. In some embodiments, PR154 can further comprise, at the 5′ end, a sequence comprising SRE007, or a reverse complement thereof, in a nanoplasmid. In some embodiments, PR153 can further comprise, at the 5′ end, a sequence comprising SRE007, or a reverse complement thereof, in a nanoplasmid. In some embodiments, PR152 can further comprise, at the 5′ end, a sequence comprising SRE007, or a reverse complement thereof, in a nanoplasmid. In some embodiments, PR151 can further comprise, at the 5′ end, a sequence comprising SRE007, or a reverse complement thereof, in a nanoplasmid. In some embodiments, PR150 can further comprise, at the 5′ end, a sequence comprising SRE007, or a reverse complement thereof, in a nanoplasmid. In some embodiments, PR131 can further comprise, at the 5′ end, a sequence comprising SRE007, or a reverse complement thereof, in a nanoplasmid. In some embodiments, SEQ ID NO: 584 can further comprise, at the 5′ end, a sequence comprising SRE007, or a reverse complement thereof, in a nanoplasmid. In some embodiments, SEQ ID NO: 585 can further comprise, at the 5′ end, a sequence comprising SRE007, or a reverse complement thereof, in a nanoplasmid. In some embodiments, SEQ ID NO: 586 can further comprise, at the 5′ end, a sequence comprising SRE007, or a reverse complement thereof, in a nanoplasmid. In some embodiments, SEQ ID NO: 587 can further comprise, at the 5′ end, a sequence comprising SRE007, or a reverse complement thereof, in a nanoplasmid. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE007, or a reverse complement thereof, in a linked double-stranded DNA. In some embodiments, PR180 can further comprise, at the 5′ end, a sequence comprising SRE007, or a reverse complement thereof, in a linked double-stranded DNA. In some embodiments, PR179 can further comprise, at the 5′ end, a sequence comprising SRE007, or a reverse complement thereof, in a linked double-stranded DNA. In some embodiments, PR178 can further comprise, at the 5′ end, a sequence comprising SRE007, or a reverse complement thereof, in a linked double-stranded DNA. In some embodiments, PR177 can further comprise, at the 5′ end, a sequence comprising SRE007, or a reverse complement thereof, in a linked double-stranded DNA. In some embodiments, PR176 can further comprise, at the 5′ end, a sequence comprising SRE007, or a reverse complement thereof, in a linked double-stranded DNA. In some embodiments, PR175 can further comprise, at the 5′ end, a sequence comprising SRE007, or a reverse complement thereof, in a linked double-stranded DNA. In some embodiments, PR174 can further comprise, at the 5′ end, a sequence comprising SRE007, or a reverse complement thereof, in a linked double-stranded DNA. In some embodiments, PR173 can further comprise, at the 5′ end, a sequence comprising SRE007, or a reverse complement thereof, in a linked double-stranded DNA. In some embodiments, PR172 can further comprise, at the 5′ end, a sequence comprising SRE007, or a reverse complement thereof, in a linked double-stranded DNA. In some embodiments, PR171 can further comprise, at the 5′ end, a sequence comprising SRE007, or a reverse complement thereof, in a linked double-stranded DNA. In some embodiments, PR170 can further comprise, at the 5′ end, a sequence comprising SRE007, or a reverse complement thereof, in a linked double-stranded DNA. In some embodiments, PR169 can further comprise, at the 5′ end, a sequence comprising SRE007, or a reverse complement thereof, in a linked double-stranded DNA. In some embodiments, PR168 can further comprise, at the 5′ end, a sequence comprising SRE007, or a reverse complement thereof, in a linked double-stranded DNA. In some embodiments, PR167 can further comprise, at the 5′ end, a sequence comprising SRE007, or a reverse complement thereof, in a linked double-stranded DNA. In some embodiments, PR166 can further comprise, at the 5′ end, a sequence comprising SRE007, or a reverse complement thereof, in a linked double-stranded DNA. In some embodiments, PR165 can further comprise, at the 5′ end, a sequence comprising SRE007, or a reverse complement thereof, in a linked double-stranded DNA. In some embodiments, PR159 can further comprise, at the 5′ end, a sequence comprising SRE007, or a reverse complement thereof, in a linked double-stranded DNA. In some embodiments, PR156 can further comprise, at the 5′ end, a sequence comprising SRE007 and SRE010, or a reverse complement thereof, in a linked double-stranded DNA. In some embodiments, PR155 can further comprise, at the 5′ end, a sequence comprising SRE007, or a reverse complement thereof, in a linked double-stranded DNA. In some embodiments, PR154 can further comprise, at the 5′ end, a sequence comprising SRE007, or a reverse complement thereof, in a linked double-stranded DNA. In some embodiments, PR153 can further comprise, at the 5′ end, a sequence comprising SRE007, or a reverse complement thereof, in a linked double-stranded DNA. In some embodiments, PR152 can further comprise, at the 5′ end, a sequence comprising SRE007, or a reverse complement thereof, in a linked double-stranded DNA. In some embodiments, PR151 can further comprise, at the 5′ end, a sequence comprising SRE007, or a reverse complement thereof, in a linked double-stranded DNA. In some embodiments, PR150 can further comprise, at the 5′ end, a sequence comprising SRE007, or a reverse complement thereof, in a linked double-stranded DNA. In some embodiments, PR131 can further comprise, at the 5′ end, a sequence comprising SRE007, or a reverse complement thereof, in a linked double-stranded DNA. In some embodiments, SEQ ID NO: 584 can further comprise, at the 5′ end, a sequence comprising SRE007, or a reverse complement thereof, in a linked double-stranded DNA. In some embodiments, SEQ ID NO: 585 can further comprise, at the 5′ end, a sequence comprising SRE007, or a reverse complement thereof, in a linked double-stranded DNA. In some embodiments, SEQ ID NO: 586 can further comprise, at the 5′ end, a sequence comprising SRE007, or a reverse complement thereof, in a linked double-stranded DNA. In some embodiments, SEQ ID NO: 587 can further comprise, at the 5′ end, a sequence comprising SRE007, or a reverse complement thereof, in a linked double-stranded DNA. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE008, or a reverse complement thereof, in a vector. In some embodiments, PR180 can further comprise, at the 5′ end, a sequence comprising SRE008, or a reverse complement thereof, in a vector. In some embodiments, PR179 can further comprise, at the 5′ end, a sequence comprising SRE008, or a reverse complement thereof, in a vector. In some embodiments, PR178 can further comprise, at the 5′ end, a sequence comprising SRE008, or a reverse complement thereof, in a vector. In some embodiments, PR177 can further comprise, at the 5′ end, a sequence comprising SRE008, or a reverse complement thereof, in a vector. In some embodiments, PR176 can further comprise, at the 5′ end, a sequence comprising SRE008, or a reverse complement thereof, in a vector. In some embodiments, PR175 can further comprise, at the 5′ end, a sequence comprising SRE008, or a reverse complement thereof, in a vector. In some embodiments, PR174 can further comprise, at the 5′ end, a sequence comprising SRE008, or a reverse complement thereof, in a vector. In some embodiments, PR173 can further comprise, at the 5′ end, a sequence comprising SRE008, or a reverse complement thereof, in a vector. In some embodiments, PR172 can further comprise, at the 5′ end, a sequence comprising SRE008, or a reverse complement thereof, in a vector. In some embodiments, PR171 can further comprise, at the 5′ end, a sequence comprising SRE008, or a reverse complement thereof, in a vector. In some embodiments, PR170 can further comprise, at the 5′ end, a sequence comprising SRE008, or a reverse complement thereof, in a vector. In some embodiments, PR169 can further comprise, at the 5′ end, a sequence comprising SRE008, or a reverse complement thereof, in a vector. In some embodiments, PR168 can further comprise, at the 5′ end, a sequence comprising SRE008, or a reverse complement thereof, in a vector. In some embodiments, PR167 can further comprise, at the 5′ end, a sequence comprising SRE008, or a reverse complement thereof, in a vector. In some embodiments, PR166 can further comprise, at the 5′ end, a sequence comprising SRE008, or a reverse complement thereof, in a vector. In some embodiments, PR165 can further comprise, at the 5′ end, a sequence comprising SRE008, or a reverse complement thereof, in a vector. In some embodiments, PR159 can further comprise, at the 5′ end, a sequence comprising SRE008, or a reverse complement thereof, in a vector. In some embodiments, PR156 can further comprise, at the 5′ end, a sequence comprising SRE008 and SRE010, or a reverse complement thereof, in a vector. In some embodiments, PR155 can further comprise, at the 5′ end, a sequence comprising SRE008, or a reverse complement thereof, in a vector. In some embodiments, PR154 can further comprise, at the 5′ end, a sequence comprising SRE008, or a reverse complement thereof, in a vector. In some embodiments, PR153 can further comprise, at the 5′ end, a sequence comprising SRE008, or a reverse complement thereof, in a vector. In some embodiments, PR152 can further comprise, at the 5′ end, a sequence comprising SRE008, or a reverse complement thereof, in a vector. In some embodiments, PR151 can further comprise, at the 5′ end, a sequence comprising SRE008, or a reverse complement thereof, in a vector. In some embodiments, PR150 can further comprise, at the 5′ end, a sequence comprising SRE008, or a reverse complement thereof, in a vector. In some embodiments, PR131 can further comprise, at the 5′ end, a sequence comprising SRE008, or a reverse complement thereof, in a vector. In some embodiments, SEQ ID NO: 584 can further comprise, at the 5′ end, a sequence comprising SRE008, or a reverse complement thereof, in a vector. In some embodiments, SEQ ID NO: 585 can further comprise, at the 5′ end, a sequence comprising SRE008, or a reverse complement thereof, in a vector. In some embodiments, SEQ ID NO: 586 can further comprise, at the 5′ end, a sequence comprising SRE008, or a reverse complement thereof, in a vector. In some embodiments, SEQ ID NO: 587 can further comprise, at the 5′ end, a sequence comprising SRE008, or a reverse complement thereof, in a vector. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE008, or a reverse complement thereof, in a nanoplasmid. In some embodiments, PR180 can further comprise, at the 5′ end, a sequence comprising SRE008, or a reverse complement thereof, in a nanoplasmid. In some embodiments, PR179 can further comprise, at the 5′ end, a sequence comprising SRE008, or a reverse complement thereof, in a nanoplasmid. In some embodiments, PR178 can further comprise, at the 5′ end, a sequence comprising SRE008, or a reverse complement thereof, in a nanoplasmid. In some embodiments, PR177 can further comprise, at the 5′ end, a sequence comprising SRE008, or a reverse complement thereof, in a nanoplasmid. In some embodiments, PR176 can further comprise, at the 5′ end, a sequence comprising SRE008, or a reverse complement thereof, in a nanoplasmid. In some embodiments, PR175 can further comprise, at the 5′ end, a sequence comprising SRE008, or a reverse complement thereof, in a nanoplasmid. In some embodiments, PR174 can further comprise, at the 5′ end, a sequence comprising SRE008, or a reverse complement thereof, in a nanoplasmid. In some embodiments, PR173 can further comprise, at the 5′ end, a sequence comprising SRE008, or a reverse complement thereof, in a nanoplasmid. In some embodiments, PR172 can further comprise, at the 5′ end, a sequence comprising SRE008, or a reverse complement thereof, in a nanoplasmid. In some embodiments, PR171 can further comprise, at the 5′ end, a sequence comprising SRE008, or a reverse complement thereof, in a nanoplasmid. In some embodiments, PR170 can further comprise, at the 5′ end, a sequence comprising SRE008, or a reverse complement thereof, in a nanoplasmid. In some embodiments, PR169 can further comprise, at the 5′ end, a sequence comprising SRE008, or a reverse complement thereof, in a nanoplasmid. In some embodiments, PR168 can further comprise, at the 5′ end, a sequence comprising SRE008, or a reverse complement thereof, in a nanoplasmid. In some embodiments, PR167 can further comprise, at the 5′ end, a sequence comprising SRE008, or a reverse complement thereof, in a nanoplasmid. In some embodiments, PR166 can further comprise, at the 5′ end, a sequence comprising SRE008, or a reverse complement thereof, in a nanoplasmid. In some embodiments, PR165 can further comprise, at the 5′ end, a sequence comprising SRE008, or a reverse complement thereof, in a nanoplasmid. In some embodiments, PR159 can further comprise, at the 5′ end, a sequence comprising SRE008, or a reverse complement thereof, in a nanoplasmid. In some embodiments, PR156 can further comprise, at the 5′ end, a sequence comprising SRE008 and SRE010, or a reverse complement thereof, in a nanoplasmid. In some embodiments, PR155 can further comprise, at the 5′ end, a sequence comprising SRE008, or a reverse complement thereof, in a nanoplasmid. In some embodiments, PR154 can further comprise, at the 5′ end, a sequence comprising SRE008, or a reverse complement thereof, in a nanoplasmid. In some embodiments, PR153 can further comprise, at the 5′ end, a sequence comprising SRE008, or a reverse complement thereof, in a nanoplasmid. In some embodiments, PR152 can further comprise, at the 5′ end, a sequence comprising SRE008, or a reverse complement thereof, in a nanoplasmid. In some embodiments, PR151 can further comprise, at the 5′ end, a sequence comprising SRE008, or a reverse complement thereof, in a nanoplasmid. In some embodiments, PR150 can further comprise, at the 5′ end, a sequence comprising SRE008, or a reverse complement thereof, in a nanoplasmid. In some embodiments, PR131 can further comprise, at the 5′ end, a sequence comprising SRE008, or a reverse complement thereof, in a nanoplasmid. In some embodiments, SEQ ID NO: 584 can further comprise, at the 5′ end, a sequence comprising SRE008, or a reverse complement thereof, in a nanoplasmid. In some embodiments, SEQ ID NO: 585 can further comprise, at the 5′ end, a sequence comprising SRE008, or a reverse complement thereof, in a nanoplasmid. In some embodiments, SEQ ID NO: 586 can further comprise, at the 5′ end, a sequence comprising SRE008, or a reverse complement thereof, in a nanoplasmid. In some embodiments, SEQ ID NO: 587 can further comprise, at the 5′ end, a sequence comprising SRE008, or a reverse complement thereof, in a nanoplasmid. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE008, or a reverse complement thereof, in a linked double-stranded DNA. In some embodiments, PR180 can further comprise, at the 5′ end, a sequence comprising SRE008, or a reverse complement thereof, in a linked double-stranded DNA. In some embodiments, PR179 can further comprise, at the 5′ end, a sequence comprising SRE008, or a reverse complement thereof, in a linked double-stranded DNA. In some embodiments, PR178 can further comprise, at the 5′ end, a sequence comprising SRE008, or a reverse complement thereof, in a linked double-stranded DNA. In some embodiments, PR177 can further comprise, at the 5′ end, a sequence comprising SRE008, or a reverse complement thereof, in a linked double-stranded DNA. In some embodiments, PR176 can further comprise, at the 5′ end, a sequence comprising SRE008, or a reverse complement thereof, in a linked double-stranded DNA. In some embodiments, PR175 can further comprise, at the 5′ end, a sequence comprising SRE008, or a reverse complement thereof, in a linked double-stranded DNA. In some embodiments, PR174 can further comprise, at the 5′ end, a sequence comprising SRE008, or a reverse complement thereof, in a linked double-stranded DNA. In some embodiments, PR173 can further comprise, at the 5′ end, a sequence comprising SRE008, or a reverse complement thereof, in a linked double-stranded DNA. In some embodiments, PR172 can further comprise, at the 5′ end, a sequence comprising SRE008, or a reverse complement thereof, in a linked double-stranded DNA. In some embodiments, PR171 can further comprise, at the 5′ end, a sequence comprising SRE008, or a reverse complement thereof, in a linked double-stranded DNA. In some embodiments, PR170 can further comprise, at the 5′ end, a sequence comprising SRE008, or a reverse complement thereof, in a linked double-stranded DNA. In some embodiments, PR169 can further comprise, at the 5′ end, a sequence comprising SRE008, or a reverse complement thereof, in a linked double-stranded DNA. In some embodiments, PR168 can further comprise, at the 5′ end, a sequence comprising SRE008, or a reverse complement thereof, in a linked double-stranded DNA. In some embodiments, PR167 can further comprise, at the 5′ end, a sequence comprising SRE008, or a reverse complement thereof, in a linked double-stranded DNA. In some embodiments, PR166 can further comprise, at the 5′ end, a sequence comprising SRE008, or a reverse complement thereof, in a linked double-stranded DNA. In some embodiments, PR165 can further comprise, at the 5′ end, a sequence comprising SRE008, or a reverse complement thereof, in a linked double-stranded DNA. In some embodiments, PR159 can further comprise, at the 5′ end, a sequence comprising SRE008, or a reverse complement thereof, in a linked double-stranded DNA. In some embodiments, PR156 can further comprise, at the 5′ end, a sequence comprising SRE008 and SRE010, or a reverse complement thereof, in a linked double-stranded DNA. In some embodiments, PR155 can further comprise, at the 5′ end, a sequence comprising SRE008, or a reverse complement thereof, in a linked double-stranded DNA. In some embodiments, PR154 can further comprise, at the 5′ end, a sequence comprising SRE008, or a reverse complement thereof, in a linked double-stranded DNA. In some embodiments, PR153 can further comprise, at the 5′ end, a sequence comprising SRE008, or a reverse complement thereof, in a linked double-stranded DNA. In some embodiments, PR152 can further comprise, at the 5′ end, a sequence comprising SRE008, or a reverse complement thereof, in a linked double-stranded DNA. In some embodiments, PR151 can further comprise, at the 5′ end, a sequence comprising SRE008, or a reverse complement thereof, in a linked double-stranded DNA. In some embodiments, PR150 can further comprise, at the 5′ end, a sequence comprising SRE008, or a reverse complement thereof, in a linked double-stranded DNA. In some embodiments, PR131 can further comprise, at the 5′ end, a sequence comprising SRE008, or a reverse complement thereof, in a linked double-stranded DNA. In some embodiments, SEQ ID NO: 584 can further comprise, at the 5′ end, a sequence comprising SRE008, or a reverse complement thereof, in a linked double-stranded DNA. In some embodiments, SEQ ID NO: 585 can further comprise, at the 5′ end, a sequence comprising SRE008, or a reverse complement thereof, in a linked double-stranded DNA. In some embodiments, SEQ ID NO: 586 can further comprise, at the 5′ end, a sequence comprising SRE008, or a reverse complement thereof, in a linked double-stranded DNA. In some embodiments, SEQ ID NO: 587 can further comprise, at the 5′ end, a sequence comprising SRE008, or a reverse complement thereof, in a linked double-stranded DNA. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE010, or a reverse complement thereof, in a vector. In some embodiments, PR180 can further comprise, at the 5′ end, a sequence comprising SRE010, or a reverse complement thereof, in a vector. In some embodiments, PR179 can further comprise, at the 5′ end, a sequence comprising SRE010, or a reverse complement thereof, in a vector. In some embodiments, PR178 can further comprise, at the 5′ end, a sequence comprising SRE010, or a reverse complement thereof, in a vector. In some embodiments, PR177 can further comprise, at the 5′ end, a sequence comprising SRE010, or a reverse complement thereof, in a vector. In some embodiments, PR176 can further comprise, at the 5′ end, a sequence comprising SRE010, or a reverse complement thereof, in a vector. In some embodiments, PR175 can further comprise, at the 5′ end, a sequence comprising SRE010, or a reverse complement thereof, in a vector. In some embodiments, PR174 can further comprise, at the 5′ end, a sequence comprising SRE010, or a reverse complement thereof, in a vector. In some embodiments, PR173 can further comprise, at the 5′ end, a sequence comprising SRE010, or a reverse complement thereof, in a vector. In some embodiments, PR172 can further comprise, at the 5′ end, a sequence comprising SRE010, or a reverse complement thereof, in a vector. In some embodiments, PR171 can further comprise, at the 5′ end, a sequence comprising SRE010, or a reverse complement thereof, in a vector. In some embodiments, PR170 can further comprise, at the 5′ end, a sequence comprising SRE010, or a reverse complement thereof, in a vector. In some embodiments, PR169 can further comprise, at the 5′ end, a sequence comprising SRE010, or a reverse complement thereof, in a vector. In some embodiments, PR168 can further comprise, at the 5′ end, a sequence comprising SRE010, or a reverse complement thereof, in a vector. In some embodiments, PR167 can further comprise, at the 5′ end, a sequence comprising SRE010, or a reverse complement thereof, in a vector. In some embodiments, PR166 can further comprise, at the 5′ end, a sequence comprising SRE010, or a reverse complement thereof, in a vector. In some embodiments, PR165 can further comprise, at the 5′ end, a sequence comprising SRE010, or a reverse complement thereof, in a vector. In some embodiments, PR159 can further comprise, at the 5′ end, a sequence comprising SRE010, or a reverse complement thereof, in a vector. In some embodiments, PR156 can further comprise, at the 5′ end, a sequence comprising SRE010 and SRE010, or a reverse complement thereof, in a vector. In some embodiments, PR155 can further comprise, at the 5′ end, a sequence comprising SRE010, or a reverse complement thereof, in a vector. In some embodiments, PR154 can further comprise, at the 5′ end, a sequence comprising SRE010, or a reverse complement thereof, in a vector. In some embodiments, PR153 can further comprise, at the 5′ end, a sequence comprising SRE010, or a reverse complement thereof, in a vector. In some embodiments, PR152 can further comprise, at the 5′ end, a sequence comprising SRE010, or a reverse complement thereof, in a vector. In some embodiments, PR151 can further comprise, at the 5′ end, a sequence comprising SRE010, or a reverse complement thereof, in a vector. In some embodiments, PR150 can further comprise, at the 5′ end, a sequence comprising SRE010, or a reverse complement thereof, in a vector. In some embodiments, PR131 can further comprise, at the 5′ end, a sequence comprising SRE010, or a reverse complement thereof, in a vector. In some embodiments, SEQ ID NO: 584 can further comprise, at the 5′ end, a sequence comprising SRE010, or a reverse complement thereof, in a vector. In some embodiments, SEQ ID NO: 585 can further comprise, at the 5′ end, a sequence comprising SRE010, or a reverse complement thereof, in a vector. In some embodiments, SEQ ID NO: 586 can further comprise, at the 5′ end, a sequence comprising SRE010, or a reverse complement thereof, in a vector. In some embodiments, SEQ ID NO: 587 can further comprise, at the 5′ end, a sequence comprising SRE010, or a reverse complement thereof, in a vector. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE010, or a reverse complement thereof, in a nanoplasmid. In some embodiments, PR180 can further comprise, at the 5′ end, a sequence comprising SRE010, or a reverse complement thereof, in a nanoplasmid. In some embodiments, PR179 can further comprise, at the 5′ end, a sequence comprising SRE010, or a reverse complement thereof, in a nanoplasmid. In some embodiments, PR178 can further comprise, at the 5′ end, a sequence comprising SRE010, or a reverse complement thereof, in a nanoplasmid. In some embodiments, PR177 can further comprise, at the 5′ end, a sequence comprising SRE010, or a reverse complement thereof, in a nanoplasmid. In some embodiments, PR176 can further comprise, at the 5′ end, a sequence comprising SRE010, or a reverse complement thereof, in a nanoplasmid. In some embodiments, PR175 can further comprise, at the 5′ end, a sequence comprising SRE010, or a reverse complement thereof, in a nanoplasmid. In some embodiments, PR174 can further comprise, at the 5′ end, a sequence comprising SRE010, or a reverse complement thereof, in a nanoplasmid. In some embodiments, PR173 can further comprise, at the 5′ end, a sequence comprising SRE010, or a reverse complement thereof, in a nanoplasmid. In some embodiments, PR172 can further comprise, at the 5′ end, a sequence comprising SRE010, or a reverse complement thereof, in a nanoplasmid. In some embodiments, PR171 can further comprise, at the 5′ end, a sequence comprising SRE010, or a reverse complement thereof, in a nanoplasmid. In some embodiments, PR170 can further comprise, at the 5′ end, a sequence comprising SRE010, or a reverse complement thereof, in a nanoplasmid. In some embodiments, PR169 can further comprise, at the 5′ end, a sequence comprising SRE010, or a reverse complement thereof, in a nanoplasmid. In some embodiments, PR168 can further comprise, at the 5′ end, a sequence comprising SRE010, or a reverse complement thereof, in a nanoplasmid. In some embodiments, PR167 can further comprise, at the 5′ end, a sequence comprising SRE010, or a reverse complement thereof, in a nanoplasmid. In some embodiments, PR166 can further comprise, at the 5′ end, a sequence comprising SRE010, or a reverse complement thereof, in a nanoplasmid. In some embodiments, PR165 can further comprise, at the 5′ end, a sequence comprising SRE010, or a reverse complement thereof, in a nanoplasmid. In some embodiments, PR159 can further comprise, at the 5′ end, a sequence comprising SRE010, or a reverse complement thereof, in a nanoplasmid. In some embodiments, PR156 can further comprise, at the 5′ end, a sequence comprising SRE010 and SRE010, or a reverse complement thereof, in a nanoplasmid. In some embodiments, PR155 can further comprise, at the 5′ end, a sequence comprising SRE010, or a reverse complement thereof, in a nanoplasmid. In some embodiments, PR154 can further comprise, at the 5′ end, a sequence comprising SRE010, or a reverse complement thereof, in a nanoplasmid. In some embodiments, PR153 can further comprise, at the 5′ end, a sequence comprising SRE010, or a reverse complement thereof, in a nanoplasmid. In some embodiments, PR152 can further comprise, at the 5′ end, a sequence comprising SRE010, or a reverse complement thereof, in a nanoplasmid. In some embodiments, PR151 can further comprise, at the 5′ end, a sequence comprising SRE010, or a reverse complement thereof, in a nanoplasmid. In some embodiments, PR150 can further comprise, at the 5′ end, a sequence comprising SRE010, or a reverse complement thereof, in a nanoplasmid. In some embodiments, PR131 can further comprise, at the 5′ end, a sequence comprising SRE010, or a reverse complement thereof, in a nanoplasmid. In some embodiments, SEQ ID NO: 584 can further comprise, at the 5′ end, a sequence comprising SRE010, or a reverse complement thereof, in a nanoplasmid. In some embodiments, SEQ ID NO: 585 can further comprise, at the 5′ end, a sequence comprising SRE010, or a reverse complement thereof, in a nanoplasmid. In some embodiments, SEQ ID NO: 586 can further comprise, at the 5′ end, a sequence comprising SRE010, or a reverse complement thereof, in a nanoplasmid. In some embodiments, SEQ ID NO: 587 can further comprise, at the 5′ end, a sequence comprising SRE010, or a reverse complement thereof, in a nanoplasmid. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE010, or a reverse complement thereof, in a linked double-stranded DNA. In some embodiments, PR180 can further comprise, at the 5′ end, a sequence comprising SRE010, or a reverse complement thereof, in a linked double-stranded DNA. In some embodiments, PR179 can further comprise, at the 5′ end, a sequence comprising SRE010, or a reverse complement thereof, in a linked double-stranded DNA. In some embodiments, PR178 can further comprise, at the 5′ end, a sequence comprising SRE010, or a reverse complement thereof, in a linked double-stranded DNA. In some embodiments, PR177 can further comprise, at the 5′ end, a sequence comprising SRE010, or a reverse complement thereof, in a linked double-stranded DNA. In some embodiments, PR176 can further comprise, at the 5′ end, a sequence comprising SRE010, or a reverse complement thereof, in a linked double-stranded DNA. In some embodiments, PR175 can further comprise, at the 5′ end, a sequence comprising SRE010, or a reverse complement thereof, in a linked double-stranded DNA. In some embodiments, PR174 can further comprise, at the 5′ end, a sequence comprising SRE010, or a reverse complement thereof, in a linked double-stranded DNA. In some embodiments, PR173 can further comprise, at the 5′ end, a sequence comprising SRE010, or a reverse complement thereof, in a linked double-stranded DNA. In some embodiments, PR172 can further comprise, at the 5′ end, a sequence comprising SRE010, or a reverse complement thereof, in a linked double-stranded DNA. In some embodiments, PR171 can further comprise, at the 5′ end, a sequence comprising SRE010, or a reverse complement thereof, in a linked double-stranded DNA. In some embodiments, PR170 can further comprise, at the 5′ end, a sequence comprising SRE010, or a reverse complement thereof, in a linked double-stranded DNA. In some embodiments, PR169 can further comprise, at the 5′ end, a sequence comprising SRE010, or a reverse complement thereof, in a linked double-stranded DNA. In some embodiments, PR168 can further comprise, at the 5′ end, a sequence comprising SRE010, or a reverse complement thereof, in a linked double-stranded DNA. In some embodiments, PR167 can further comprise, at the 5′ end, a sequence comprising SRE010, or a reverse complement thereof, in a linked double-stranded DNA. In some embodiments, PR166 can further comprise, at the 5′ end, a sequence comprising SRE010, or a reverse complement thereof, in a linked double-stranded DNA. In some embodiments, PR165 can further comprise, at the 5′ end, a sequence comprising SRE010, or a reverse complement thereof, in a linked double-stranded DNA. In some embodiments, PR159 can further comprise, at the 5′ end, a sequence comprising SRE010, or a reverse complement thereof, in a linked double-stranded DNA. In some embodiments, PR156 can further comprise, at the 5′ end, a sequence comprising SRE010 and SRE010, or a reverse complement thereof, in a linked double-stranded DNA. In some embodiments, PR155 can further comprise, at the 5′ end, a sequence comprising SRE010, or a reverse complement thereof, in a linked double-stranded DNA. In some embodiments, PR154 can further comprise, at the 5′ end, a sequence comprising SRE010, or a reverse complement thereof, in a linked double-stranded DNA. In some embodiments, PR153 can further comprise, at the 5′ end, a sequence comprising SRE010, or a reverse complement thereof, in a linked double-stranded DNA. In some embodiments, PR152 can further comprise, at the 5′ end, a sequence comprising SRE010, or a reverse complement thereof, in a linked double-stranded DNA. In some embodiments, PR151 can further comprise, at the 5′ end, a sequence comprising SRE010, or a reverse complement thereof, in a linked double-stranded DNA. In some embodiments, PR150 can further comprise, at the 5′ end, a sequence comprising SRE010, or a reverse complement thereof, in a linked double-stranded DNA. In some embodiments, PR131 can further comprise, at the 5′ end, a sequence comprising SRE010, or a reverse complement thereof, in a linked double-stranded DNA. In some embodiments, SEQ ID NO: 584 can further comprise, at the 5′ end, a sequence comprising SRE010, or a reverse complement thereof, in a linked double-stranded DNA. In some embodiments, SEQ ID NO: 585 can further comprise, at the 5′ end, a sequence comprising SRE010, or a reverse complement thereof, in a linked double-stranded DNA. In some embodiments, SEQ ID NO: 586 can further comprise, at the 5′ end, a sequence comprising SRE010, or a reverse complement thereof, in a linked double-stranded DNA. In some embodiments, SEQ ID NO: 587 can further comprise, at the 5′ end, a sequence comprising SRE010, or a reverse complement thereof, in a linked double-stranded DNA. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE012, or a reverse complement thereof, in a vector. In some embodiments, PR180 can further comprise, at the 5′ end, a sequence comprising SRE012, or a reverse complement thereof, in a vector. In some embodiments, PR179 can further comprise, at the 5′ end, a sequence comprising SRE012, or a reverse complement thereof, in a vector. In some embodiments, PR178 can further comprise, at the 5′ end, a sequence comprising SRE012, or a reverse complement thereof, in a vector. In some embodiments, PR177 can further comprise, at the 5′ end, a sequence comprising SRE012, or a reverse complement thereof, in a vector. In some embodiments, PR176 can further comprise, at the 5′ end, a sequence comprising SRE012, or a reverse complement thereof, in a vector. In some embodiments, PR175 can further comprise, at the 5′ end, a sequence comprising SRE012, or a reverse complement thereof, in a vector. In some embodiments, PR174 can further comprise, at the 5′ end, a sequence comprising SRE012, or a reverse complement thereof, in a vector. In some embodiments, PR173 can further comprise, at the 5′ end, a sequence comprising SRE012, or a reverse complement thereof, in a vector. In some embodiments, PR172 can further comprise, at the 5′ end, a sequence comprising SRE012, or a reverse complement thereof, in a vector. In some embodiments, PR171 can further comprise, at the 5′ end, a sequence comprising SRE012, or a reverse complement thereof, in a vector. In some embodiments, PR170 can further comprise, at the 5′ end, a sequence comprising SRE012, or a reverse complement thereof, in a vector. In some embodiments, PR169 can further comprise, at the 5′ end, a sequence comprising SRE012, or a reverse complement thereof, in a vector. In some embodiments, PR168 can further comprise, at the 5′ end, a sequence comprising SRE012, or a reverse complement thereof, in a vector. In some embodiments, PR167 can further comprise, at the 5′ end, a sequence comprising SRE012, or a reverse complement thereof, in a vector. In some embodiments, PR166 can further comprise, at the 5′ end, a sequence comprising SRE012, or a reverse complement thereof, in a vector. In some embodiments, PR165 can further comprise, at the 5′ end, a sequence comprising SRE012, or a reverse complement thereof, in a vector. In some embodiments, PR159 can further comprise, at the 5′ end, a sequence comprising SRE012, or a reverse complement thereof, in a vector. In some embodiments, PR156 can further comprise, at the 5′ end, a sequence comprising SRE012, or a reverse complement thereof, in a vector. In some embodiments, PR155 can further comprise, at the 5′ end, a sequence comprising SRE012, or a reverse complement thereof, in a vector. In some embodiments, PR154 can further comprise, at the 5′ end, a sequence comprising SRE012, or a reverse complement thereof, in a vector. In some embodiments, PR153 can further comprise, at the 5′ end, a sequence comprising SRE012, or a reverse complement thereof, in a vector. In some embodiments, PR152 can further comprise, at the 5′ end, a sequence comprising SRE012, or a reverse complement thereof, in a vector. In some embodiments, PR151 can further comprise, at the 5′ end, a sequence comprising SRE012, or a reverse complement thereof, in a vector. In some embodiments, PR150 can further comprise, at the 5′ end, a sequence comprising SRE012, or a reverse complement thereof, in a vector. In some embodiments, PR131 can further comprise, at the 5′ end, a sequence comprising SRE012, or a reverse complement thereof, in a vector. In some embodiments, SEQ ID NO: 584 can further comprise, at the 5′ end, a sequence comprising SRE012, or a reverse complement thereof, in a vector. In some embodiments, SEQ ID NO: 585 can further comprise, at the 5′ end, a sequence comprising SRE012, or a reverse complement thereof, in a vector. In some embodiments, SEQ ID NO: 586 can further comprise, at the 5′ end, a sequence comprising SRE012, or a reverse complement thereof, in a vector. In some embodiments, SEQ ID NO: 587 can further comprise, at the 5′ end, a sequence comprising SRE012, or a reverse complement thereof, in a vector. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE012, or a reverse complement thereof, in a nanoplasmid. In some embodiments, PR180 can further comprise, at the 5′ end, a sequence comprising SRE012, or a reverse complement thereof, in a nanoplasmid. In some embodiments, PR179 can further comprise, at the 5′ end, a sequence comprising SRE012, or a reverse complement thereof, in a nanoplasmid. In some embodiments, PR178 can further comprise, at the 5′ end, a sequence comprising SRE012, or a reverse complement thereof, in a nanoplasmid. In some embodiments, PR177 can further comprise, at the 5′ end, a sequence comprising SRE012, or a reverse complement thereof, in a nanoplasmid. In some embodiments, PR176 can further comprise, at the 5′ end, a sequence comprising SRE012, or a reverse complement thereof, in a nanoplasmid. In some embodiments, PR175 can further comprise, at the 5′ end, a sequence comprising SRE012, or a reverse complement thereof, in a nanoplasmid. In some embodiments, PR174 can further comprise, at the 5′ end, a sequence comprising SRE012, or a reverse complement thereof, in a nanoplasmid. In some embodiments, PR173 can further comprise, at the 5′ end, a sequence comprising SRE012, or a reverse complement thereof, in a nanoplasmid. In some embodiments, PR172 can further comprise, at the 5′ end, a sequence comprising SRE012, or a reverse complement thereof, in a nanoplasmid. In some embodiments, PR171 can further comprise, at the 5′ end, a sequence comprising SRE012, or a reverse complement thereof, in a nanoplasmid. In some embodiments, PR170 can further comprise, at the 5′ end, a sequence comprising SRE012, or a reverse complement thereof, in a nanoplasmid. In some embodiments, PR169 can further comprise, at the 5′ end, a sequence comprising SRE012, or a reverse complement thereof, in a nanoplasmid. In some embodiments, PR168 can further comprise, at the 5′ end, a sequence comprising SRE012, or a reverse complement thereof, in a nanoplasmid. In some embodiments, PR167 can further comprise, at the 5′ end, a sequence comprising SRE012, or a reverse complement thereof, in a nanoplasmid. In some embodiments, PR166 can further comprise, at the 5′ end, a sequence comprising SRE012, or a reverse complement thereof, in a nanoplasmid. In some embodiments, PR165 can further comprise, at the 5′ end, a sequence comprising SRE012, or a reverse complement thereof, in a nanoplasmid. In some embodiments, PR159 can further comprise, at the 5′ end, a sequence comprising SRE012, or a reverse complement thereof, in a nanoplasmid. In some embodiments, PR156 can further comprise, at the 5′ end, a sequence comprising SRE012, or a reverse complement thereof, in a nanoplasmid. In some embodiments, PR155 can further comprise, at the 5′ end, a sequence comprising SRE012, or a reverse complement thereof, in a nanoplasmid. In some embodiments, PR154 can further comprise, at the 5′ end, a sequence comprising SRE012, or a reverse complement thereof, in a nanoplasmid. In some embodiments, PR153 can further comprise, at the 5′ end, a sequence comprising SRE012, or a reverse complement thereof, in a nanoplasmid. In some embodiments, PR152 can further comprise, at the 5′ end, a sequence comprising SRE012, or a reverse complement thereof, in a nanoplasmid. In some embodiments, PR151 can further comprise, at the 5′ end, a sequence comprising SRE012, or a reverse complement thereof, in a nanoplasmid. In some embodiments, PR150 can further comprise, at the 5′ end, a sequence comprising SRE012, or a reverse complement thereof, in a nanoplasmid. In some embodiments, PR131 can further comprise, at the 5′ end, a sequence comprising SRE012, or a reverse complement thereof, in a nanoplasmid. In some embodiments, SEQ ID NO: 584 can further comprise, at the 5′ end, a sequence comprising SRE012, or a reverse complement thereof, in a nanoplasmid. In some embodiments, SEQ ID NO: 585 can further comprise, at the 5′ end, a sequence comprising SRE012, or a reverse complement thereof, in a nanoplasmid. In some embodiments, SEQ ID NO: 586 can further comprise, at the 5′ end, a sequence comprising SRE012, or a reverse complement thereof, in a nanoplasmid. In some embodiments, SEQ ID NO: 587 can further comprise, at the 5′ end, a sequence comprising SRE012, or a reverse complement thereof, in a nanoplasmid. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, PR181 can further comprise, at the 5′ end, a sequence comprising SRE012, or a reverse complement thereof, in a linked double-stranded DNA. In some embodiments, PR180 can further comprise, at the 5′ end, a sequence comprising SRE012, or a reverse complement thereof, in a linked double-stranded DNA. In some embodiments, PR179 can further comprise, at the 5′ end, a sequence comprising SRE012, or a reverse complement thereof, in a linked double-stranded DNA. In some embodiments, PR178 can further comprise, at the 5′ end, a sequence comprising SRE012, or a reverse complement thereof, in a linked double-stranded DNA. In some embodiments, PR177 can further comprise, at the 5′ end, a sequence comprising SRE012, or a reverse complement thereof, in a linked double-stranded DNA. In some embodiments, PR176 can further comprise, at the 5′ end, a sequence comprising SRE012, or a reverse complement thereof, in a linked double-stranded DNA. In some embodiments, PR175 can further comprise, at the 5′ end, a sequence comprising SRE012, or a reverse complement thereof, in a linked double-stranded DNA. In some embodiments, PR174 can further comprise, at the 5′ end, a sequence comprising SRE012, or a reverse complement thereof, in a linked double-stranded DNA. In some embodiments, PR173 can further comprise, at the 5′ end, a sequence comprising SRE012, or a reverse complement thereof, in a linked double-stranded DNA. In some embodiments, PR172 can further comprise, at the 5′ end, a sequence comprising SRE012, or a reverse complement thereof, in a linked double-stranded DNA. In some embodiments, PR171 can further comprise, at the 5′ end, a sequence comprising SRE012, or a reverse complement thereof, in a linked double-stranded DNA. In some embodiments, PR170 can further comprise, at the 5′ end, a sequence comprising SRE012, or a reverse complement thereof, in a linked double-stranded DNA. In some embodiments, PR169 can further comprise, at the 5′ end, a sequence comprising SRE012, or a reverse complement thereof, in a linked double-stranded DNA. In some embodiments, PR168 can further comprise, at the 5′ end, a sequence comprising SRE012, or a reverse complement thereof, in a linked double-stranded DNA. In some embodiments, PR167 can further comprise, at the 5′ end, a sequence comprising SRE012, or a reverse complement thereof, in a linked double-stranded DNA. In some embodiments, PR166 can further comprise, at the 5′ end, a sequence comprising SRE012, or a reverse complement thereof, in a linked double-stranded DNA. In some embodiments, PR165 can further comprise, at the 5′ end, a sequence comprising SRE012, or a reverse complement thereof, in a linked double-stranded DNA. In some embodiments, PR159 can further comprise, at the 5′ end, a sequence comprising SRE012, or a reverse complement thereof, in a linked double-stranded DNA. In some embodiments, PR156 can further comprise, at the 5′ end, a sequence comprising SRE012, or a reverse complement thereof, in a linked double-stranded DNA. In some embodiments, PR155 can further comprise, at the 5′ end, a sequence comprising SRE012, or a reverse complement thereof, in a linked double-stranded DNA. In some embodiments, PR154 can further comprise, at the 5′ end, a sequence comprising SRE012, or a reverse complement thereof, in a linked double-stranded DNA. In some embodiments, PR153 can further comprise, at the 5′ end, a sequence comprising SRE012, or a reverse complement thereof, in a linked double-stranded DNA. In some embodiments, PR152 can further comprise, at the 5′ end, a sequence comprising SRE012, or a reverse complement thereof, in a linked double-stranded DNA. In some embodiments, PR151 can further comprise, at the 5′ end, a sequence comprising SRE012, or a reverse complement thereof, in a linked double-stranded DNA. In some embodiments, PR150 can further comprise, at the 5′ end, a sequence comprising SRE012, or a reverse complement thereof, in a linked double-stranded DNA. In some embodiments, PR131 can further comprise, at the 5′ end, a sequence comprising SRE012, or a reverse complement thereof, in a linked double-stranded DNA. In some embodiments, SEQ ID NO: 584 can further comprise, at the 5′ end, a sequence comprising SRE012, or a reverse complement thereof, in a linked double-stranded DNA. In some embodiments, SEQ ID NO: 585 can further comprise, at the 5′ end, a sequence comprising SRE012, or a reverse complement thereof, in a linked double-stranded DNA. In some embodiments, SEQ ID NO: 586 can further comprise, at the 5′ end, a sequence comprising SRE012, or a reverse complement thereof, in a linked double-stranded DNA. In some embodiments, SEQ ID NO: 587 can further comprise, at the 5′ end, a sequence comprising SRE012, or a reverse complement thereof, in a linked double-stranded DNA. In some embodiments, any of these named elements can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a nucleic acid having any of these named elements and any of SEQ ID NOs: 584-587 can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, the disclosure provides for a nucleic acid comprising any of the sequences described herein separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, the nucleic acid can comprise any of the sequences listed in Table 1B or any one of the sequences listed in Table 1J separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, a sequence comprising any of nucleic acid sequences listed in Table 1B and any one of the core promoter sequences listed in Table 1J can be separated by a linker of variable length, wherein the linker can comprise a sequence of 1, 2, 5, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

### EXAMPLES

These examples are provided for illustrative purposes only and not to limit the scope of the claims provided herein.

### Example 1: Development of a High-Throughput Screening Platform for Novel Cancer-Activated Promoters

In this example, a high-throughput screening (HTS) platform to design and test synthetic sequence elements that can drive cancer specific expression of a report gene or a gene of interest. Synthetic promoters described herein comprise a core promoter and one or more response elements. Response elements can be designed by tiling binding sites for putative transcription factor candidates identified through transcriptomics and proteomics. Using Massively Parallel Reporter Assay (MPRA) method, 1,800 unique synthetic response elements placed in front of (5′ end of) the two different core promoters were screened. Synthetic promoters were able to drive expression up to 80 times higher than the previously described FOS-coreBIRC5 synthetic promoter. In addition, TF tiles for TCF7 (a downstream target of the WNT signaling pathway) and TP53 (a tumor suppressor that is mutated in many cancers) that can drive expression 100 times or more within a specific lung cancer cell line that represents a specific pathway dysregulation were identified. The MPRA platform allows simultaneously testing thousands of hypotheses from the multi-omics identification of key transcription factors in cancer combined with different design strategies for a functioning response element, as demonstrated in this example. Low-throughput validation demonstrated that the MPRA accurately identifies winning candidates from thousands of test sequences. This MPRA pipeline is a key component of the workflow to develop and test hypotheses for cancer-regulated gene expression at a massive, highly parallelized scale. The MPRA can be performed by assembling a pooled library of reporter plasmids that interrogate the function of a candidate DNA sequence through an expressed barcode. The pool of reporter plasmids can be transfected into mammalian cell lines and then harvested for RNA. The barcodes from the mRNA and the input DNA can be sequenced using Next Generation sequencing techniques. The input DNA barcode can be used to normalize the mRNA barcode to get the final expression level for each candidate DNA sequence.

Genes are highly regulated by a complex collaboration between the transcription factors downstream of signaling pathways and the DNA regulatory elements they interact with. These DNA regulatory elements include promoters, 5′ and 3′UTRs, and distal and proximal enhancers. Cancer is marked by aberrant molecular signaling leading to highly active transcription factors and functional signaling cascades that might normally only be found in early development or in other disease states, leading to hallmark cancer phonotypes such as uncontrolled growth and invasion/metastasis. The regulatory elements of these dysregulated genes can be re-used in exogenous vectors to drive expression that is restricted to cancer cells. For example, the promoters for Survivin and hTERT have been used exogenously to drive tumor specific expression. Although endogenous promoters can be used as cancer-activated regulatory elements, by having highly complex logic and interplay of multiple transcription factor binding sites, they can be unpredictable and have higher basal activity than desired. Endogenous promoters also rarely drive very high signal even in the correct cell-state or genomic profile to activate TFs, as few natural promoters have been naturally evolved to have the high level of expression observed in the constitutive viral-origin promoters often used in gene therapy.

A stronger, and more predictably activated promoter can be engineered by bringing together diverse regulatory elements that respond to a variety of signaling pathways that might not be found in a single regulatory element. For these reasons, a synthetic approach has been developed to construct novel cancer-activated promoters, as further described in Example 2.

Synthetic promoters were constructed by combining a small core promoter from a gene upregulated in cancer with synthetic response elements to particular dysregulated TFs. These response elements comprise a series of repeated binding sites for the desired TFs. Various “-omics” based approaches have been used to identify TFs that are enriched in tumor targets, and hundreds of possible candidate TFs have been identified. Each of those TFs has many possible binding sites and configurations that can create the most efficacious response element. As testing each individual candidate element in series can be costly in labor and time, a high-throughput approach was used to test thousands of synthetic promoter elements simultaneously.

The screening assay that most closely aligns with the vector design and transient delivery platform described herein is the MPRA (Massively Parallel Reporter Assay). In this assay, short oligos containing a sequence of interest coupled with a unique barcode was synthesized and cloned as a pool into a reporter plasmid. This plasmid pool was transfected into a cell line and the expression of each sequence of interest was measured in parallel through targeted barcode sequencing of the RNA and plasmid DNA. MPRAs have been used to identify endogenous human enhancers, determine the role of genetic variation on gene expression, and characterize sequence determinants of gene regulation. This screening assay is an ideal method to simultaneously test and identify synthetic promoters that drive strong expression in relevant cancer models.

A high-throughput screening platform (MPRA) to identify novel synthetic promoters that can drive cancer-activated expression is described in this example.

### High-Throughput Screening (HTS) Methodology

### Overview

The MPRA was performed by assembling a pooled library of reporter plasmids that interrogate the function of a candidate DNA sequence through an expressed barcode. The pool of reporter plasmids was transfected into mammalian cell lines and then harvested for RNA. The barcodes from the mRNA and the input DNA were sequenced using Next Generation sequencing (NGS) techniques. The input DNA barcode was used to normalize the mRNA barcode to get the final expression level for each candidate DNA sequence.

### Homotypic TF Tile Library Design

A computational pipeline that systematically creates synthetic DNA sequences that contain repeated TF binding sites (TF tiles) was developed using the following parameters:

1. Total Length: The full length of the synthetic DNA sequence. A length of 140 bp was used.

2. Total Number of Binding Sites in a Tile: The number of repeated binding sites that make up the homotypic TF tile. 6 repeated binding sites were used.

3. Spacing: The number of nucleotides between each of the TF binding sites. 0, 3, 7, and 10 bp spacing were used.

4. Binding Site Sequence: The binding site sequences for each tile were chosen using the TF's position frequency matrix (PFM) from either the HOMER or JASPAR database. The pipeline used the frequency of each nucleotide at each position and chose the most frequent nucleotide or nucleotides based on a user defined frequency cut off. Once a nucleotide was chosen for one position all other positions were assigned the most frequent nucleotide. The pipeline used a 10% cut off and focused on the positions at the core of the motif. For example, if at the center position the frequency of A, T, C, G is 5%, 5%, 30%, 60%, respectively, then two binding sites were chosen. One would have a C and the other would have a G and all other positions would have the highest frequency nucleotide.

In addition, the pipeline has the following features:

1. Length Consistency: For TF tiles that were shorter than the total length, a small filler sequence was added to the 5′ end. This short sequence was randomly chosen from a 1 kb filler sequence that was manually curated to reduce strong binding site for characterized TFs. This created synthetic DNA sequences that were the same length with little to no effect on the overall expression.

2. Restriction Enzyme Check: Each synthetic DNA sequence was checked for restriction enzyme cut sites used in the cloning method. In this example, the KpnI and XbaI cut sites were used and checked.

3. Addition of Cloning Sequences: Primer sites and restriction enzyme sites were added to facilitate the cloning workflow.

4. Addition of Barcodes: A unique barcode was added to each synthetic DNA sequence. These barcodes were created using the DNABarcodes R package. This package created large numbers of barcodes that were different enough from each other that when mutations were introduced during the sequencing and library preparation the barcodes were still distinguishable.

Using the pipeline described above, homotypic TF Tiles for 77 Lung adenocarcinoma (LUAD) specific TFs were designed. These TF were computationally identified using various multiomic data sets, including RNA-seq and proteomics (see Example 2). A full list of TFs can be found in Table 1D-1I. 24 TF tiles were designed for each TF (6 binding site variations each with 4 different spacing variants: 0, 3, 7, 10 bp). Each tile was assigned 6 barcodes for a total of 144 DNA sequences for each TF. Additionally, positive expression controls and controls for the baseline core promoter expression were included. The positive expression controls include FOSL and Canscript (see Example 2), and 90 barcodes were assigned to each. Baseline expression controls comprised 5 different 140 bp segments of the filler sequence (curated to remove all strong TF binding sites) that were assigned 30 barcodes for a total of 150. An oligo pool of ˜12,000 oligos containing the synthetic TF tile, the assigned barcode, and necessary sequences for cloning was ordered from a vendor (TWIST BIOSICENCES).

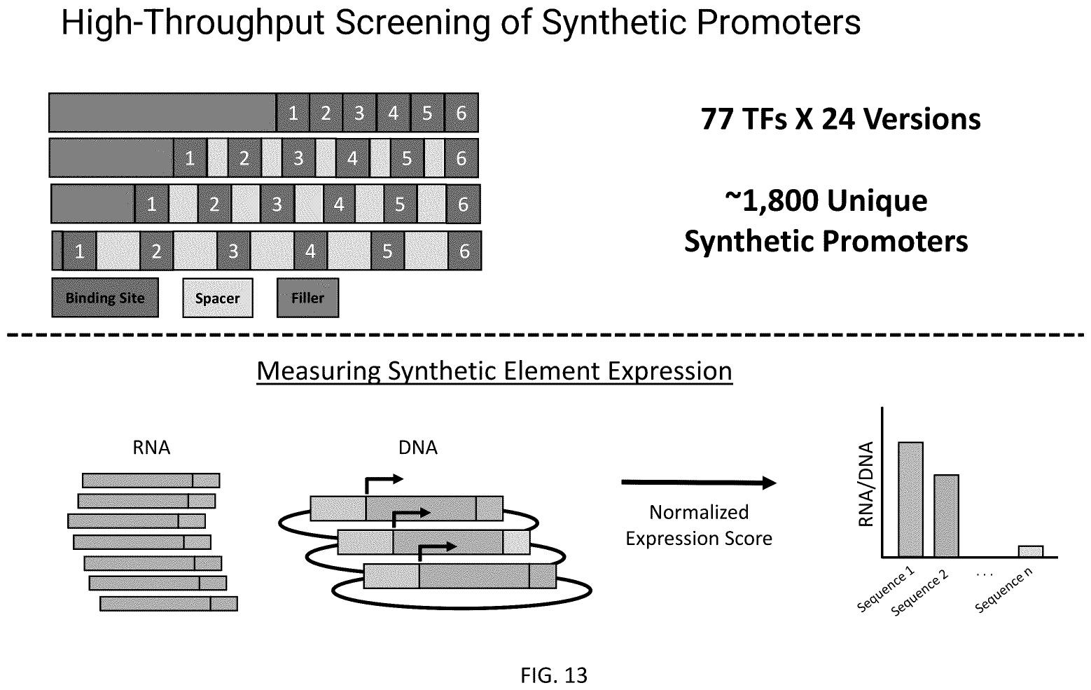

FIG. 13 (top) shows each synthetic DNA sequence that was designed as a series of repeated transcription factor (TF) binding sites derived from the consensus binding motif for the TF of interest (blue). To test the impact of the different relative positioning of these sites around the helical nature of the double stranded DNA (one helical turn is equivalent to ˜10.5 base pairs), the repeated binding sites were separated by a variable length of nucleic acid spacer sequences (FIG. 13, yellow). Lastly, the synthetic DNA sequence contained a short filler sequence (FIG. 13, grey) to maintain consistent total length of the candidate enhancer sequence block.

### Building the MPRA Library

### Base Plasmid

A base plasmid that contains the key features necessary for cloning, mammalian expression, and transfection efficiency monitoring was constructed. The plasmid has SfiI restriction enzyme sites for cloning in synthetic oligos, and a reverse selection cassette for removing undesired cloning products. For mammalian expression, the plasmid has a strong polyA termination site downstream of (or 3′ to) where the final expression cassette will be located. There is an additional polyA termination site upstream of (or 5′ to) the final expression cassette that reduces errant transcripts that might be produced by the bacterial components of the plasmid. Lastly, a constitutively expressed GFP cassette was added to monitor the transfection efficiency either visually under a fluorescent microscope or using FACS.

### Cloning Round 1: Oligo Pool

The single stranded oligo pool was PCR amplified to create a pool of double stranded DNA fragments. To maintain the integrity of the library (size and complexity), an emulsion PCR with a limited number of cycles ranging from 12-20 cycles was used. Next the base plasmid and double stranded DNA pool were digested with the SfiI restriction enzyme. The base plasmid was gel extracted using the QIAGEN II Gel Extraction Kit. The double stranded DNA pool was purified using the Monarch PCR and DNA Cleanup Kit. The digested products were ligated overnight using a T4 DNA ligase and electroporated into bacteria at a recovery efficiency of at least 100 times the complexity (number of unique DNA sequences) of the oligo library. The integrity of the library was validated by performing Sanger sequencing on 40 individual clones. All clones that were Sanger sequenced contained a unique sequence from the oligo pool, indicating that the library's complexity was maintained. In addition, there was only 1 sequenced clone that contained a large variation in the sequence, indicating an estimated error rate of less than 3%, which met the tolerated criteria. The bacteria pool was cultured overnight at 30° C., and a plasmid prep was done using the ZymoPURE II Plasmid Maxiprep Kit. The product was a plasmid pool containing the library of synthetic sequences. Each of these sequences contained the XbaI and KpnI restriction enzyme sites. These sites were used in the next round of cloning to add in the core promoter and luciferase expression.

### Cloning Round 2:

The plasmid pool from the Round 1 cloning was serially digested with KpnI and XbaI. Each digestion was purified using the Monarch PCR and DNA Cleanup Kit. The final digested product was treated with CIP to dephosphorylate the overhangs. Additionally, plasmids containing the coreBIRC5-Fluc or the TATA-TSS-Fluc cassette were digested with KpnI and XbaI, and gel extracted using a standard kit. The digested plasmid pool and core promoters were ligated overnight and electroporated into bacteria at a recovery efficiency of at least 100 times the complexity of the oligo library. 10 single clones were Sangar sequenced to validate the integrity of the library and expression cassette. Each of the clones sequenced had an intact core promoter-luciferase expression cassette and the expected TF tile-barcode combination. The pools of bacteria were cultured, and the plasmid libraries were extracted using a standard maxiprep kit.

### Transfections and Library Preparation

### Cell Line Transfections

Each library was transfected independently at least 3 times (3 replicates) in various lung cancer model cell lines, including the well-studied H1299 and several patient-derived xenografts (PDXs) from human lung tumors. Cells for each line were seeded at appropriate densities on 6-well plates. The total number of cells seeded was at least 100 times the complexity of the library and scaled for the typical transfection efficiency of the relevant cell line. For example, with the library complexity of 12,000 and a cell line of a transfection efficiency of 75%, 1.6e6 cells total were seeded for each replicate. Cells were transfected using the commercial product Lipofectamine 3000 and harvested after 24 or 48 hours depending on the cell viability. Before harvesting, the transfection efficiency was evaluated by visual inspection of GFP expression using a fluorescent microscope. If the transfection efficiency was lower than expected, it was repeated.

### NGS Tag-Seq Library Prep

Total RNA was extracted using a standard Trizol prep method. Briefly, cells from each replicate were resuspended in Trizol, chloroform was added, and the mixtures were phase-separated using centrifugation. Then, the aqueous layer was removed, and total RNA was recovered using ethanol precipitation. Next, mRNA was isolated using a commercial polyA magnet bead kit (Dynabeads mRNA Purification Kit), followed by a commercially available Turbo DNase treatment to remove all DNA fragments, including the transfected plasmid. To ensure that samples did not contain residual plasmid DNA, a pre-NGS PCR was performed using 30-50 ng of mRNA for 26 cycles and the result was visualized on a gel. Samples that had a visual band underwent additional DNase treatments. Next, cDNA production was done using the commercially available Superscript IV. 400-600 ng of mRNA was used with a poly-dT primer. Targeted PCR amplification was performed to produce an Illumina compatible NGS sequencing library that contained the TF tile associated barcodes. In parallel, NGS sequencing libraries was also produced from the input plasmid DNA library. Indexed libraries were pooled, and paired end sequenced on an Illumina sequencing platform.

### Data Processing and Analysis

Barcodes were matched to their respective synthetic TF tiles using the DNABarcodes R package. All libraries had greater than 95% of the sequenced barcodes matched to it synthetic TF tile. To determine the expression scores for our screens, the MPRAnalyze R package was used. Briefly, this package uses a graphical model to relate the barcode counts from the RNA to barcode counts from the input plasmid DNA. It supports the use of multiple barcodes per sequence, multiple replicates, and multiple conditions (i.e., cell line).

### Luciferase Assay

For the low throughput validation, cells were transfected using Lipofectamine 3000 according to the manufacturer's instructions. Briefly, for each well, 100 ng of plasmid DNA was mixed with 0.2 μL of P3000 reagent and 0.2 μL of Lipofectamine 3000 and 2 ng of control DNA in 100 μL Opti-MEM medium and the mixture was incubated at room temperature for 20 minutes. The transfection mixture was added to the cells in a 96-well plate and incubated for 24 hours. Approximately 24 hours after transfection, the firefly luciferase and renilla luciferase levels were measured from each well using the Promega Dual-Glo Luciferase System (E2940) with a working volume of 50 μL.

### Results

### Study Design and Synthetic TF Tile Construction

A high-throughput MPRA screen for identifying synthetic regulatory elements that drive strong expression in lung cancer has been developed and validated. In the first high-throughput screen, the focus was on screening synthetic enhancer elements intended to serve as response elements to TFs that play a role in non-small cell lung cancer (NSCLC). A multi-omics approach to NSCLC identified more than 100 TFs that are dysregulated in lung adenocarcinoma (LUAD). Based on the strength of the multi-omics and evidence, and with the filter of DNA binding site characterization, 77 TFs were selected for this library. For each TF, 24,140 bp homotypic tiles that varied in the binding site motif and the spacing between the binding sites were designed. Each binding site motif was tiled 6 times. 6 different binding site motifs with 4 spacing variants (0, 3, 7, and 10 bp) were chosen. 6 barcodes were assigned, and 4 different control TF tiles were also included (FOSL1, TTF, MYC-MAX, Cansript). As a result, a total of 1,850 unique synthetic sequences were designed and constructed.

These unique enhancer sequences were placed in front of (e.g., upstream of or 5′ end of) two core promoters and screened. The two core promoters included the minimal TATA-TSS that drives little to no expression of a reporter gene or a gene of interest, and coreBIRC5 that drives cancer specific expression of a reporter gene or a gene of interest (see Example 1). Additionally, 5 control sequences were included. The control sequences were selected from random sequences and known not to contain TF binding sites and served as negative control, when combined with the core promoters, and the measurement of expression from control sequences were used as the baseline expression. Several positive control TF tiles were also used. These positive control TF tiles had been previously characterized (i.e., FOSL2) (see Example 2). To add redundancy and allow for statistical significance, each TF tile was assigned 6 barcodes for a total screening library size of 12,000.

The coreBIRC5 and TATA-TSS libraries were screened in four lung cancer cell line models: H1299 and three human patient derived xenograft (PDX) tumor cell lines (LXFA586, LXFL1121, and LXFL430). At least 3 biological replicates were performed for each cell line. To measure the activity of the synthetic TF tiles, the detected barcode levels in the RNA were normalized to the DNA input, to calculate an expression score (as described in the Methods above).

### High-Throughput Screen Identifies Active Synthetic TF Tiles

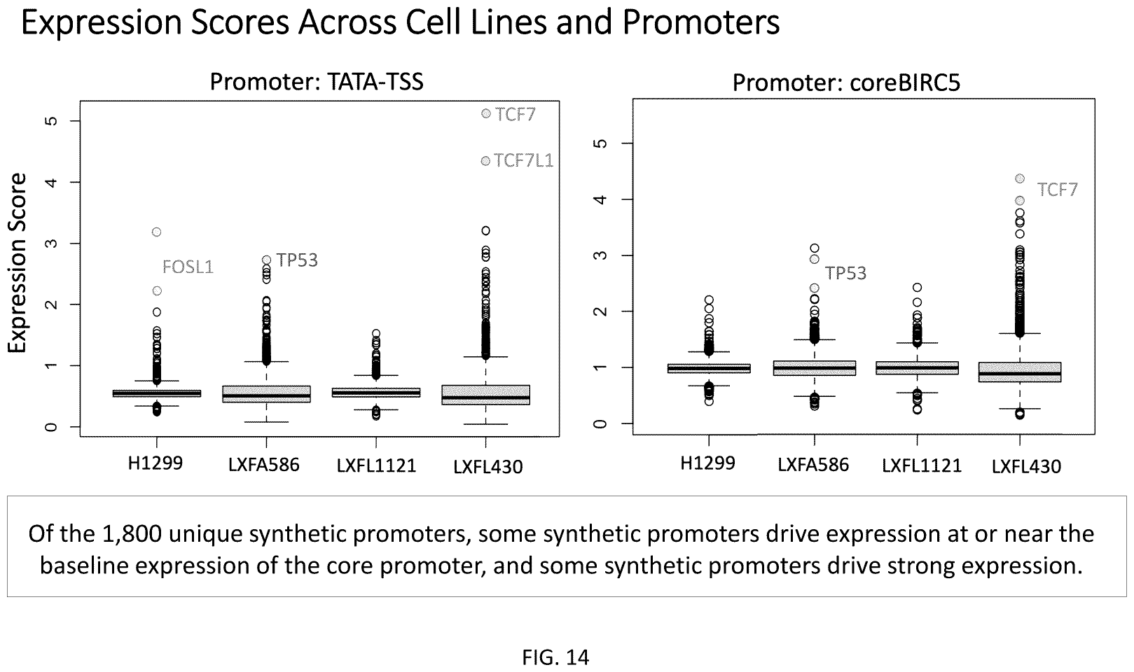

In both first two screening libraries, synthetic enhancers were found to drive expression in cancer cell line models with both the TATA-TSS and coreBIRC5 core promoters. The expression score distribution varied between cell lines, with the PDX LXFL430 having the widest distribution and the highest expression scores (FIG. 14).

Next, the fold change for each unique synthetic sequence was calculated using the baseline core promoter expression score to normalize. With the TATA-TSS core promoter driving low levels of expression, these TF tiles had a higher fold change compared to the coreBIRC5 promoter. The positive control FOSL2 tile was strongly active in the H1299 cell line for both core promoters tested, suggesting that there are no candidates that are stronger than the FOS motif for H1299s in this library of dysregulated TFs. Other synthetic response elements were discovered in this approach that were highly active in all cell lines. These include CREB3L1, TWIST, and a set of HOX variants (MNX1, HOXC10, HOXB9).

Other tiles were much more specific for particular genetic backgrounds across different cell lines. For example, the TCF7 and TCF7L1 TF tiles ranked at the top of the list in the LXFL430 cell line but not in any other cell lines. Similarly, the TP53 TF tiles rank highly only in the LXFA586 cell line.

Some TF tiles were found to have a core promoter preference. For example, the TWIST_v3 tile is at the top of the ranked list for the coreBIRC5 promoter but is not highly ranked for the TATA-TSS promoter. Additionally, this TWIST_v3 tile is ranked highly in all cell lines. HOXC10, MNX1, and CREB3L1 tile variants were also ranked higher for two or more cell lines (Table 1D-1I).

### Synthetic TF Tile Validation

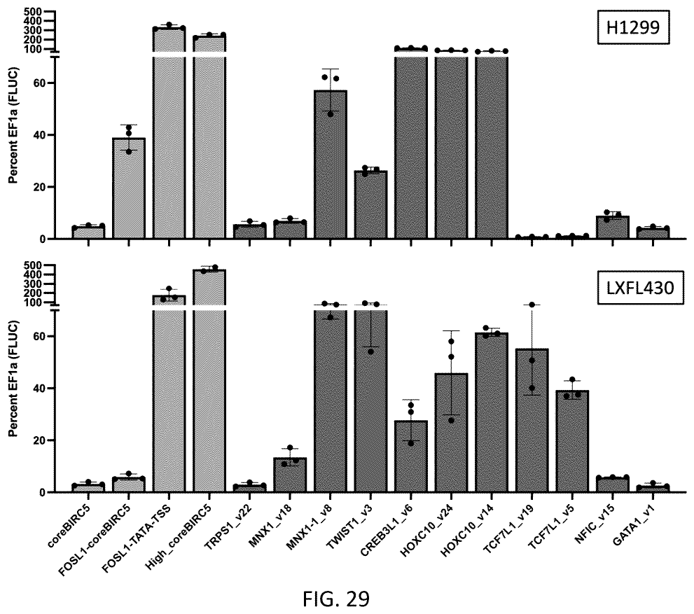

To establish the validity of the screening strategy and qualify candidates for further testing, a set of high-scoring and low-scoring candidates from the screen was constructed using the coreBIRC5 core sequence in the PDX430 lung cancer cell line. The candidates were cloned into the luciferase reporter plasmid and the expression of the luciferase was measured. Most of the high-scoring enhancer sequences were also found to have expression level that is higher than the core sequence alone, with some candidates approaching levels of internal positive control promoters, FOS-TATA-TSS and High-coreBIRC5 (FIG. 29). In PDX-derived cell line LXFL430, 10 out of 11 TF tiles tested from the top of the list drove significantly higher expression than coreBIRC5 alone (FIG. 29), while only 1 out of 9 sequences tested from the bottom of the list drove expression higher than coreBIRC5.

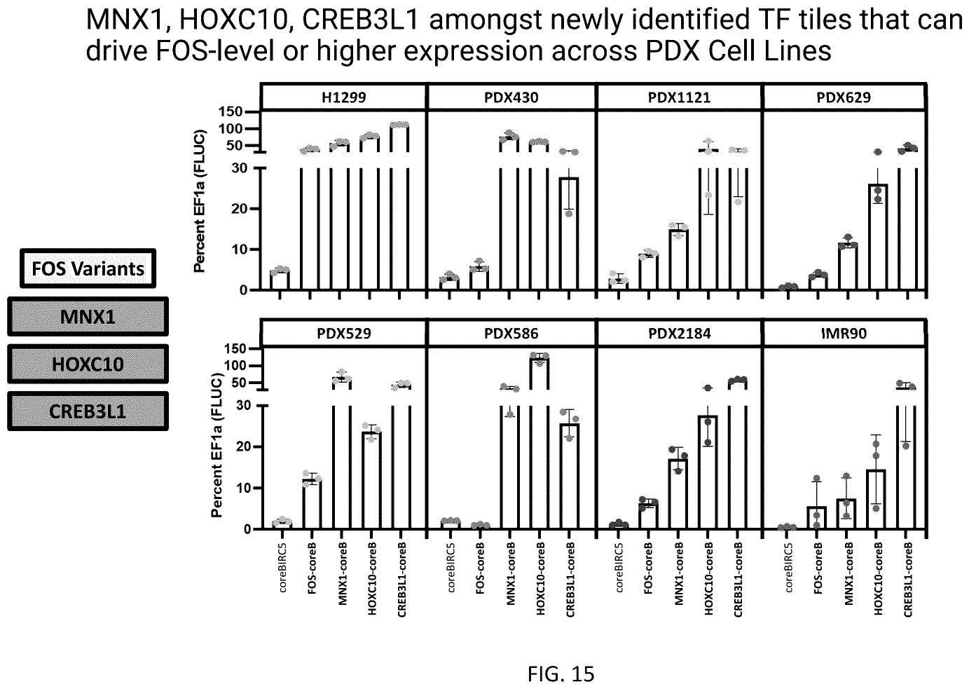

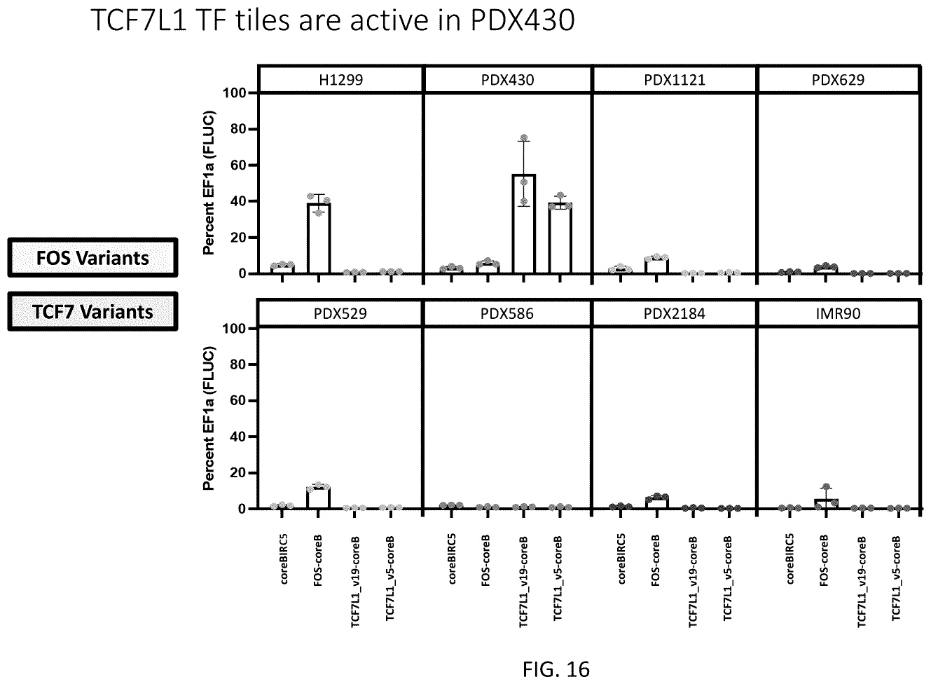

In summary, more than seven unique TFs were identified as candidates for synthetic enhancers that can drive cancer-regulated gene expression through the two screens described in this example. Some of the candidates appear to be stronger than the previous favorite FOSL2-enhancer element and will be studied further. As shown in FIG. 15, new synthetic promoters comprising coreBIRC5, that responds to HOXC10, MNX1, and CREB3L1, drive stronger expression of the reporter gene than the FOS-coreBIRC5 promoter.

### Conclusion

MPRA high-throughput has been successfully implemented to screen 1,800 unique TF tiles in combination with two separate TF tile libraries, one using the TATA-TSS promoter and the other using the coreBIRC5 promoter. These libraries were screened in five different lung cancer cell lines. As expected, most candidate response elements drove expression of a reporter gene similar to the baseline expression of the core promoter alone, supporting the importance of approaching this testing in a highly parallel manner. However, a subset of synthetic promoter elements that drive expression well above the core promoter baseline was identified, as demonstrated by the screening data and low-throughput validation. Synthetic response elements particularly responding to HOXC10, CREB3L1 and MNX1 were found to drive expression across multiple lung cancer cell lines. For example, the HOXC10 element drove the expression of a reporter gene up to 80 times higher than FOS-coreBIRC5 synthetic promoter.

In addition, synthetic response elements that uniquely drive expression in only specific genetic contexts were identified. The screen identified that multiple variations of elements responding to TCF7 or TP53 drove strong expression in only LXFL430 or LXFA586, respectively. Low-throughput validation confirmed the results and have led to designing and testing of combining multiple pathway-sensitive synthetic promoter elements into a single regulatory element. TCF7 is the downstream target of the B-cat/Wnt signaling pathway, which is well-studied in primary & metastatic lung cancer. TP53 is also a well-studied for its role, particularly in mutated form, within non-small cell lung cancer.

Overall, the screening platform successfully identified synthetic promoters that (1) drive expression of a gene broadly across lung cancer models due to universal changes in proliferation and de-differentiation and (2) are downstream of signaling pathways and drive expression in specific lung cancer models. The MPRA developed is a core feature in designing and constructing synthetic promoters, given the vast amount of sequence space to cover when designing completely new promoter sequences from scratch. As demonstrated here, it allows simultaneously testing thousands of hypotheses from the multi-omics identification of key TFs in cancer combined with different design strategies for a functioning response element. The MPRA accurately brings the best candidates to the top, as demonstrated by the low-throughput validation results, and thus can greatly accelerate designing novel synthetic promoters. This MPRA platform, now optimized and fully-developed, can also be applied to test any series of large hypotheses that can result in stronger expression of a gene in any models of choice, such as mutations to UTR sequences, ideal codon optimization, or screening a library of endogenous enhancer sequences.

### Example 2: Design and Construction of Synthetic Promoters

In this example, the general strategy of synthetic promoter engineering to combine specific response elements in dysregulated pathways in cancer is described. The modular components (response element, signal element and core promoter) can be individually and synchronously engineered for improved sensitivity, specificity and signal strength in both low-throughput and high-throughput approaches. Response of synthetic promoters to distinct TF upregulation is demonstrated, which indicates that synthetic promoters described herein can establish highly predictable activity in new cell lines.

The cancer-activated promoter is a key component within cancer-activated DNA constructs to drive expression of a synthetic biomarker in cancer cells. Cancer is notably characterized by aberrant molecular signaling, which is a result of dysregulated expression of highly active transcription factors (TFs) and functional signaling cascades that can normally only be found in early development or in other disease states. Synthetic promoters described herein can function directly as response elements or sensors for known dysregulated transcription factors. Synthetic promoters can perform as protein sensors by responding predictably to the presence of phosphorylated TF in the nucleus. This can allow estimating sensitivity and specificity using available in silico data for cancer and normal patients, without having to create and test in empirical models. Empirical testing can follow to demonstrate the responsiveness of a synthetic promoter comprising TF binding sequences to the TF, which allows extrapolating known expression data for that TF in large datasets like The Cancer Genome Atlas (TCGA) or Clinical Proteomic Tumor Analysis Consortium (CPTAC). In addition, as there are no common models for benign tissues, proteomics and transcriptomics of benign lung disease can be studied to determine whether a TF is present, which can be helpful for predicting whether a synthetic promoter comprising the TF binding sequence can activate in those cell states.

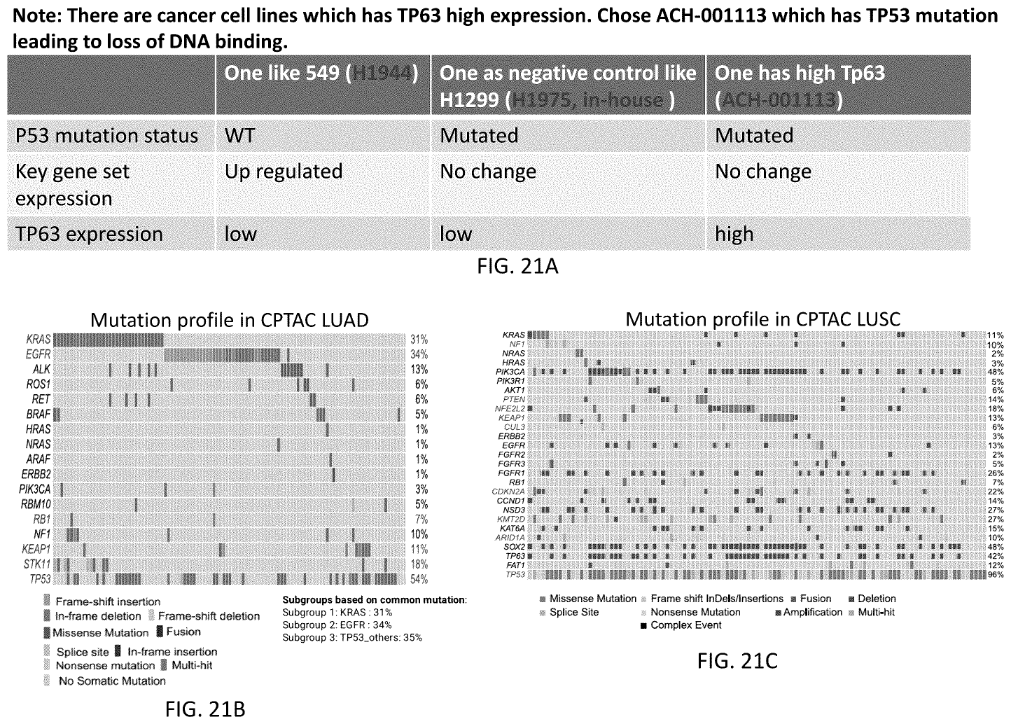

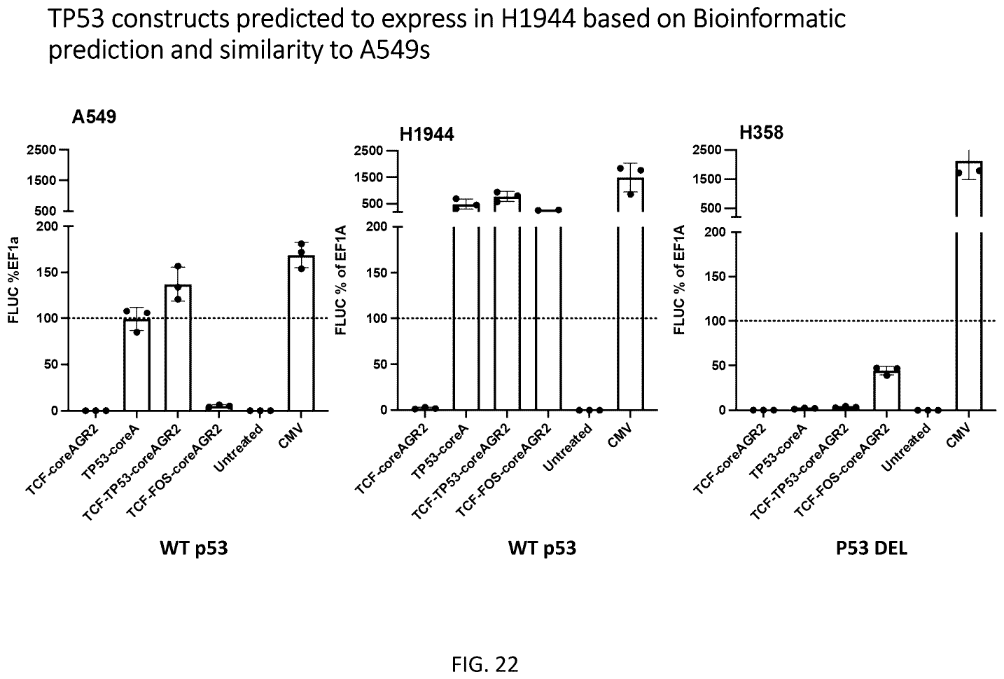

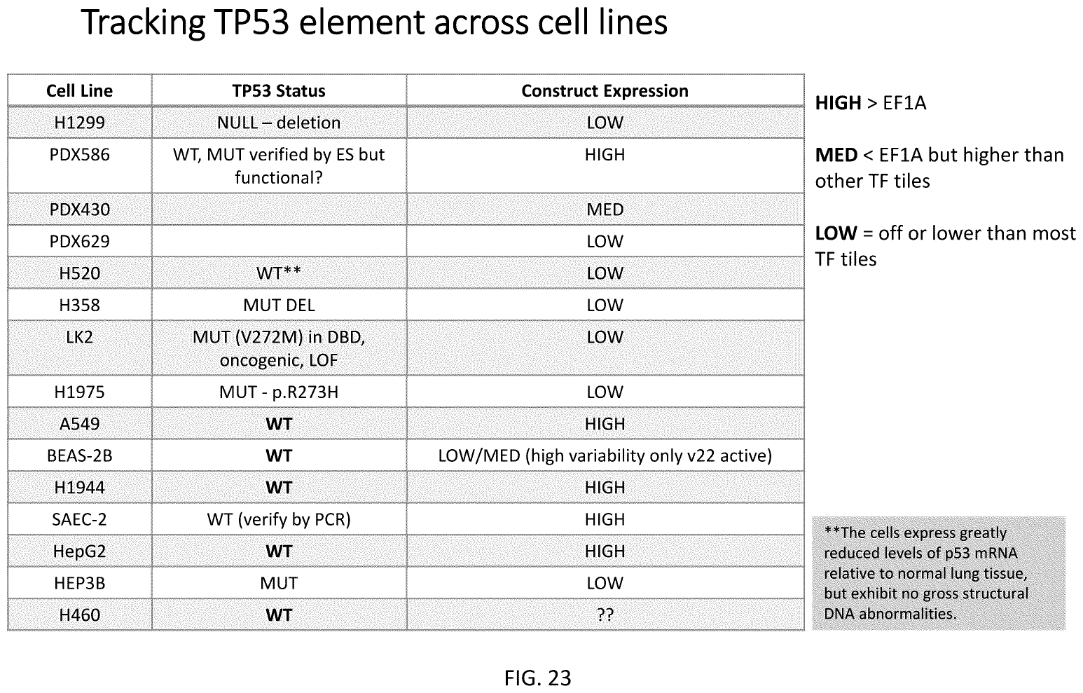

The approach to designing cancer-specific promoters starts with identifying the key response elements that bind the TFs. These TFs were identified by a multi-omics approach that utilizes transcriptomics, proteomics and phospho-proteomics to identify TFs that are highly upregulated in cancer cells or tissues, compared to normal cells or tissues. TFs identified using the multi-omics approach in non-small cell lung cancer (NSCLC) were categorized by major driver mutations and signaling pathways (FIG. 21B). TFs identified are downstream of major NSCLC driver mutations (e.g., EGFR, KRAS, TP53, etc.) and signaling pathways. Combining specific elements across multiple pathways can ensure broad cancer coverage of cancer specific expression of a reporter gene or a gene of interest. For example, based on the above analysis, a synthetic promoter can be designed to include elements to ensure coverage of LUAD and LUSC dysregulated pathways by combining elements and probing various signaling pathways.

To build a synthetic promoter, one can use the known DNA binding site (TFBS) as a sequence element to “sense” that TF's presence, and if present, that TF upon binding to the promoter, will recruit additional transcriptional machinery and co-factors such as RNA polymerase. There are also additional signal-based elements that are not cancer-specific, but generally can attract more transcriptional machinery to a promoter that has been activated.

The transcription start site (TSS) is the driving component of the core promoter. Two approaches have been used to design the core: (1) using a minimal basal promoter, which is frequently used to create response elements and (2) using the core region of a cancer-specific promoter, which adds additional specificity to the construct. The three components—cancer-activated response elements, signal elements, and cancer-specific cores—are each modular and highly engineerable.

### Synthetic Construct Design and Cloning

### Core Promoters

A minimal cancer-specific core promoter can comprise a short DNA sequence within the promoter region of a gene that is specifically activated or repressed in cancer cells compared to normal cells. The core promoter region is a critical regulatory element that controls the initiation of transcription by RNA polymerase II. The coreBIRC5 element comprises a 74 bp element from the 3′ end of the promoter consisting of a TP53 half-site, and 33 bp after the transcriptional start site (TSS).

Equivalent types of core promoter sequences were also created for endogenous promoters AGR2, CST1, and FAM111B by evaluating candidate sequences in the UCSC Genome Browser and limiting assessment from −300 bp to +100 bp relative to the predicted TSS of the endogenous promoter. Boundaries of the core sequences were further trimmed based on a combination of the following: presence of ChIP-Seq peaks (including general TFs and indicators of active promoter regions such as RNA Pol II, DNAse I, H3K4me1, H3K4me3 peaks), TFs that may indicate cancer specificity by presence in cancer cell lines and absence in non-cancerous cell lines, abundance of predicted TFBS via JASPAR or HOMER motif analysis, and/or retaining regions of high species conservation.

The TATA-TSS minimal core (37 bp) comprises a canonical TATA site with a 23 bp GC-rich spacer 5′ end to or upstream of the TSS, which can mediate high expression.

### Tiled Transcription Factor Binding Sites

JASPAR (open-access database of curated and non-redundant transcription factor (TF) binding profiles from six different taxonomic groups) consensus sequences were used as the DNA binding domain and tiled consecutively or with a 3 bp spacer between the DNA binding domains to fill a size of 125 bp. Ultramers were ordered from Integrated DNA Technologies (IDT) with a common sequence at the 3′ end. Single-stranded ultramers were PCR-amplified using a common reverse primer to add appropriate restriction enzyme digestion sites as described below. Ultramer sequences are listed in Table 2.

TABLE 2
Ultramer sequences

| SEQ ID | | | |
|---|---|---|---|
| NO. | Reference | Sequence Name | Sequence |
| 344 | 312398676 | TTF-1_1_no space | AAT AGG TAC CAC TAG TGG TTT TGT GGG |
| | | | GTT TTG TGG GGT TTT GTG GGG TTT TGT |
| | | | GGG GTT TTG TGG GGT TTT GTG GGG TTT |
| | | | TGT GGG GTT TTG TGG GGT TTT GTG GGG |
| | | | TTT TGT GGT GCG CTC CCG ACA TGC CCC |
| | | | GC |
| 345 | 312398677 | MAX MYC no | AAT AGG TAC CAC TAG TAG TTC AAC ACG |
| | | space | TGG TCT GGG AGT TCA ACA CGT GGT CTG |
| | | | GGA GTT CAA CAC GTG GTC TGG GAG TTC |
| | | | AAC ACG TGG TCT GGG AGT TCA ACA CGT |
| | | | GGT CTG GGT GCG CTC CCG ACA TGC CCC |
| | | | GC |
| 346 | 312398678 | TTF-1_1_3bp space | AAT AGG TAC CAC TAG TGG TTT TGT GGA |
| | | | GAG GTT TTG TGG TCG GGT TTT GTG GGA |
| | | | CGG TTT TGT GGC TAG GTT TTG TGG ACT |
| | | | GGT TTT GTG GTG CGG TTT TGT GGG TAG |
| | | | GTT TTG TGG TGC GCT CCC GAC ATG CCC |
| | | | CGC |
| 347 | 312398679 | MAX_MYC_3bp | AAT AGG TAC CAC TAG TAG TTC AAC ACG |
| | | space | TGG TCT GGG AGA AGT TCA ACA CGT GGT |
| | | | CTG GGT CGA GTT CAA CAC GTG GTC TGG |
| | | | GGA CAG TTC AAC ACG TGG TCT GGG CTA |
| | | | AGT TCA ACA CGT GGT CTG GGT GCG CTC |
| | | | CCG ACA TGC CCC GC |
| 348 | 312398680 | TTF-1_2_no space | AAT AGG TAC CAC TAG TAG CCA CTT GAA |
| | | | ATT AGC CAC TTG AAA TTA GCC ACT TGA |
| | | | AAT TAG CCA CTT GAA ATT AGC CAC TTG |
| | | | AAA TTA GCC ACT TGA AAT TAG CCA CTT |
| | | | GAA ATT TGC GCT CCC GAC ATG CCC CGC |
| 349 | 312398681 | GATA6_no space | AAT AGG TAC CAC TAG TGA CAG ATA AGA |
| | | | AAG ACA GAT AAG AAA GAC AGA TAA GAA |
| | | | AGA CAG ATA AGA AAG ACA GAT AAG AAA |
| | | | GAC AGA TAA GAA AGA CAG ATA AGA AAG |
| | | | ACA GAT AAG AAA TGC GCT CCC GAC ATG |
| | | | CCC CGC |
| 350 | 312398682 | TTF-1_2_3bp space | AAT AGG TAC CAC TAG TAG CCA CTT GAA |
| | | | ATT AGA AGC CAC TTG AAA TTT CGA GCC |
| | | | ACT TGA AAT TGA CAG CCA CTT GAA ATT |
| | | | CTA AGC CAC TTG AAA TTA CTA GCC ACT |
| | | | TGA AAT TTG CGC TCC CGA CAT GCC CCG C |
| 351 | 312398683 | GATA6_3bp space | AAT AGG TAC CAC TAG TGA CAG ATA AGA |
| | | | AAA GAG ACA GAT AAG AAA TCG GAC AGA |
| | | | TAA GAA AGA CGA CAG ATA AGA AAC TAG |
| | | | ACA GAT AAG AAA ACT GAC AGA TAA GAA |
| | | | ATG CGA CAG ATA AGA AAT GCG CTC CCG |
| | | | ACA TGC CCC GC |
| 352 | 312398684 | TTF-1_3_no space | AAT AGG TAC CAC TAG TCT GGG AAC AAG |
| | | | TGC TGG GAA CAA GTG CTG GGA ACA AGT |
| | | | GCT GGG AAC AAG TGC TGG GAA CAA GTG |
| | | | CTG GGA ACA AGT GCT GGG AAC AAG TGC |
| | | | TGG GAA CAA GTG TGC GCT CCC GAC ATG |
| | | | CCC CGC |
| 353 | 312398685 | GATA1_no space | AAT AGG TAC CAC TAG TTT CTA ATC TAT |
| | | | TTC TAA TCT ATT TCT AAT CTA TTT CTA |
| | | | ATC TAT TTC TAA TCT ATT TCT AAT CTA |
| | | | TTT CTA ATC TAT TTC TAA TCT ATT TCT |
| | | | AAT CTA TTG CGC TCC CGA CAT GCC CCG C |
| 354 | 312398686 | TTF-1_3_3bp space | AAT AGG TAC CAC TAG TCT GGG AAC AAG |
| | | | TGA GAC TGG GAA CAA GTG TCG CTG GGA |
| | | | ACA AGT GGA CCT GGG AAC AAG TGC TAC |
| | | | TGG GAA CAA GTG ACT CTG GGA ACA AGT |
| | | | GTG CCT GGG AAC AAG TGT GCG CTC CCG |
| | | | ACA TGC CCC GC |
| 355 | 312398687 | GATA1_3bp space | AAT AGG TAC CAC TAG TTT CTA ATC TAT |
| | | | AGA TTC TAA TCT ATT CGT TCT AAT CTA |
| | | | TGA CTT CTA ATC TAT CTA TTC TAA TCT |
| | | | ATA CTT TCT AAT CTA TTG CTT CTA ATC |
| | | | TAT TGC GCT CCC GAC ATG CCC CGC |
| 356 | 312398688 | TTF-1_4_no space | AAT AGG TAC CAC TAG TGA CTC CTC AAG |
| | | | GGG ACT CCT CAA GGG GAC TCC TCA AGG |
| | | | GGA CTC CTC AAG GGG ACT CCT CAA GGG |
| | | | GAC TCC TCA AGG GGA CTC CTC AAG GGG |
| | | | ACT CCT CAA GGG TGC GCT CCC GAC ATG |
| | | | CCC CGC |
| 357 | 312398689 | FOSL 1_no space | AAT AGG TAC CAC TAG TGG TGA CTC ATG |
| | | | GGT GAC TCA TGG GTG ACT CAT GGG TGA |
| | | | CTC ATG GGT GAC TCA TGG GTG ACT CAT |
| | | | GGG TGA CTC ATG GGT GAC TCA TGG GTG |
| | | | ACT CAT GTG CGC TCC CGA CAT GCC CCG C |
| 358 | 312398690 | TTF-1_4_3bp space | AAT AGG TAC CAC TAG TGA CTC CTC AAG |
| | | | GGA GAG ACT CCT CAA GGG TCG GAC TCC |
| | | | TCA AGG GGA CGA CTC CTC AAG GGC TAG |
| | | | ACT CCT CAA GGG ACT GAC TCC TCA AGG |
| | | | GTG CGA CTC CTC AAG GGT GCG CTC CCG |
| | | | ACA TGC CCC GC |
| 359 | 312398691 | FOSL 1_3bp space | AAT AGG TAC CAC TAG TGG TGA CTC ATG |
| | | | AGA GGT GAC TCA TGT CGG GTG ACT CAT |
| | | | GGA CGG TGA CTC ATG CTA GGT GAC TCA |
| | | | TGA CTG GTG ACT CAT GTG CGG TGA CTC |
| | | | ATG TGC GCT CCC GAC ATG CCC CGC |
| 360 | 312398692 | TCF7_no space | AAT AGG TAC CAC TAG TCG GGC TTT GAT |
| | | | CTT TCG GGC TTT GAT CTT TCG GGC TTT |
| | | | GAT CTT TCG GGC TTT GAT CTT TCG GGC |
| | | | TTT GAT CTT TCG GGC TTT GAT CTT TCG |
| | | | GGC TTT GAT CTT TTG CGC TCC CGA CAT |
| | | | GCC CCG C |
| 361 | 312398693 | STAT3_no space | AAT AGG TAC CAC TAG TCT TCT GGG AAA |
| | | | CTT CTG GGA AAC TTC TGG GAA ACT TCT |
| | | | GGG AAA CTT CTG GGA AAC TTC TGG GAA |
| | | | ACT TCT GGG AAA CTT CTG GGA AAC TTC |
| | | | TGG GAA ATG CGC TCC CGA CAT GCC CCG C |
| 362 | 312398694 | TCF7_3bp space | AAT AGG TAC CAC TAG TCG GGC TTT GAT |
| | | | CTT TAG ACG GGC TTT GAT CTT TTC GCG |
| | | | GGC TTT GAT CTT TGA CCG GGC TTT GAT |
| | | | CTT TCT ACG GGC TTT GAT CTT TAC TCG |
| | | | GGC TTT GAT CTT TTG CGC TCC CGA CAT |
| | | | GCC CCG C |
| 363 | 312398695 | STAT3_3bp space | AAT AGG TAC CAC TAG TCT TCT GGG AAA |
| | | | AGA CTT CTG GGA AAT CGC TTC TGG GAA |
| | | | AGA CCT TCT GGG AAA CTA CTT CTG GGA |
| | | | AAA CTC TTC TGG GAA ATG CCT TCT GGG |
| | | | AAA TGC GCT CCC GAC ATG CCC CGC |
| 364 | 312398696 | TCF7: L2_no space | AAT AGG TAC CAC TAG TGC GCT TTG ATG |
| | | | TGC GGG GCG GCC CTT TGA AGT TGG CGC |
| | | | TTT GAT GTG CGG GGC GGC CCT TTG AAG |
| | | | TTG GCG CTT TGA TGT GCG GGG CGG CCC |
| | | | TTT GAA GTT GTG CGC TCC CGA CAT GCC |
| | | | CCG |
| 365 | 312398697 | STAT: STAT no | AAT AGG TAC CAC TAG TAA TTC TTA GAA |
| | | space | ATA AAT TCT TAG AAA TAA ATT CTT AGA |
| | | | AAT AAA TTC TTA GAA ATA AAT TCT TAG |
| | | | AAA TAA ATT CTT AGA AAT AAA TTC TTA |
| | | | GAA ATA TGC GCT CCC GAC ATG CCC CGC |
| 366 | 312398698 | TCF7: L2_3bp | AAT AGG TAC CAC TAG TGC GCT TTG ATG |
| | | space | TGC GGG GCG GCC CTT TGA AGT TGA GAG |
| | | | CGC TTT GAT GTG CGG GGC GGC CCT TTG |
| | | | AAG TTG TCG GCG CTT TGA TGT GCG GGG |
| | | | CGG CCC TTT GAA GTT GTG CGC TCC CGA |
| | | | CAT GCC CCG C |
| 367 | 312398699 | STAT: STAT_3bp | AAT AGG TAC CAC TAG TAA TTC TTA GAA |
| | | space | ATA AGA AAT TCT TAG AAA TAT CGA ATT |
| | | | CTT AGA AAT AGA CAA TTC TTA GAA ATA |
| | | | CTA AAT TCT TAG AAA TAA CTA ATT CTT |
| | | | AGA AAT ATG CGC TCC CGA CAT GCC CCG C |
| 368 | 312398700 | MSC_no space | AAT AGG TAC CAC TAG TAA CAG CTG TTA |
| | | | ACA GCT GTT AAC AGC TGT TAA CAG CTG |
| | | | TTA ACA GCT GTT AAC AGC TGT TAA CAG |
| | | | CTG TTA ACA GCT GTT AAC AGC TGT TTG |
| | | | CGC TCC CGA CAT GCC CCG C |
| 369 | 312398701 | SOX9_no space | AAT AGG TAC CAC TAG TAA AAC AAA GGA |
| | | | TCC TTT GTT TTA AAA CAA AGG ATC CTT |
| | | | TGT TTT AAA ACA AAG GAT CCT TTG TTT |
| | | | TAA AAC AAA GGA TCC TTT GTT TTA AAA |
| | | | CAA AGG ATC CTT TGT TTT TGC GCT CCC |
| | | | GAC ATG CCC CGC |
| 370 | 312398702 | MSC_3bp space | AAT AGG TAC CAC TAG TAA CAG CTG TTA |
| | | | GAA ACA GCT GTT TCG AAC AGC TGT TGA |
| | | | CAA CAG CTG TTC TAA ACA GCT GTT ACT |
| | | | AAC AGC TGT TTG CAA CAG CTG TTG TAA |
| | | | ACA GCT GTT TGC GCT CCC GAC ATG CCC |
| | | | CGC |
| 371 | 312398703 | SOX9_3bp space | AAT AGG TAC CAC TAG TAA AAC AAA GGA |
| | | | TCC TTT GTT TTA GAA AAA CAA AGG ATC |
| | | | CTT TGT TTT TCG AAA ACA AAG GAT CCT |
| | | | TTG TTT TGA CAA AAC AAA GGA TCC TTT |
| | | | GTT TTT GCG CTC CCG ACA TGC CCC GC |
| 372 | 312398704 | ZEB1_no space | AAT AGG TAC CAC TAG TCA CCT GCA CCT |
| | | | GCA CCT GCA CCT GCA CCT GCA CCT GCA |
| | | | CCT GCA CCT GCA CCT GCA CCT GCA CCT |
| | | | GCA CCT GTG CGC TCC CGA CAT GCC CCG C |
| 373 | 312398705 | HNF4_no space | AAT AGG TAC CAC TAG TAA AGT CCA AGT |
| | | | CCA AAA GTC CAA GTC CAA AAG TCC AAG |
| | | | TCC AAA AGT CCA AGT CCA AAA GTC CAA |
| | | | GTC CAA AAG TCC AAG TCC AAA AGT CCA |
| | | | AGT CCA TGC GCT CCC GAC ATG CCC CGC |
| 374 | 312398706 | ZEB1_3bp space | AAT AGG TAC CAC TAG TCA CCT GAG ACA |
| | | | CCT GTC GCA CCT GGA CCA CCT GCT ACA |
| | | | CCT GAC TCA CCT GTG CCA CCT GAG ACA |
| | | | CCT GTC GCA CCT GGA CCA CCT GTG CGC |
| | | | TCC CGA CAT GCC CCG C |
| 375 | 312398707 | HNF4_3bp space | AAT AGG TAC CAC TAG TAA AGT CCA AGT |
| | | | CCA AGA AAA GTC CAA GTC CAT CGA AAG |
| | | | TCC AAG TCC AGA CAA AGT CCA AGT CCA |
| | | | CTA AAA GTC CAA GTC CAA CTA AAG TCC |
| | | | AAG TCC ATG CGC TCC CGA CAT GCC CCG |
| | | | C |
| 376 | 312398708 | BIRC5_core REV | CCA TGG TGG CTT TAC CAA CAG TAC CGG |
| | | | ATT GCC AAG CTT GGC CGC CGA GGC CAG |
| | | | ATC TTG ATA TCC TCG AGG CTA GCC CAC |
| | | | CTC TGC CAA CGG GTC CCG CGA CTC AAA |
| | | | TCT GGC GGT TAA TGG CGC GCC GCG GGG |
| | | | CAT GTC GGG AGC GCA GGT ACC G |

Cloning into Firefly Reporter Vector

To generate a reporter construct for use in measuring promoter activity, DNA fragments of interest were cloned into a standard Firefly Luciferase (FLUC) reporter vector from Promega (pGL4.10[luc2] Promega E6651). Two cloning methods were used: restriction enzyme cloning and Gibson assembly.

For restriction enzyme cloning, DNA fragments containing promoter sequences were amplified by PCR using primers designed to incorporate KpnI and NheI restriction enzyme recognition sites in the PCR products. The PCR products were then digested with the appropriate restriction enzymes, purified using gel extraction kits (Zymo Cat #D4001), and ligated into the FLUC vector that had been digested with the same enzymes using NEB Quick Ligation Kit (Cat #M2200). The ligation mixture was transformed into E. coli Stable cells (C3040H), and clones were screened by restriction enzyme digestion and DNA sequencing to confirm the correct insert.

For Gibson assembly, Gibson Assembly Master Mix (NEB E2611) was used. Briefly, PCR products containing the promoter of interest and the FLUC vector were generated using primers designed to create overlapping regions between the two fragments. The PCR products were then mixed with Gibson Assembly Master Mix and incubated at 50° C. for 1 hour. The resulting mixture was then transformed into E. coli Stable cells, and clones were screened by DNA sequencing to confirm the correct assembly.

DNA was scaled up and purified using QIAGEN Plasmid Plus Midi (Cat #12945) or equivalent. Briefly, larger cultures were prepared from bacterial glycerol stocks containing the plasmid DNA. A 2 mL culture was started in the morning and larger cultures inoculated for overnight growth at 37° C. Purified DNA was used for subsequent in vitro and in vivo transfections.

### Cell Lines

Cells were maintained according to standard protocols with recommended media described below and incubated at 37° C. and 5% CO₂. H1299 (human non-small cell lung carcinoma cell line derived from the lymph node), H520 (squamous cell carcinoma), and LK-2 (squamous cell carcinoma) cells were cultured in standard RPMI1640 medium supplemented with 10% (v/v) fetal bovine serum. IMR90 (normal lung fibroblast cell line) cells were cultured in standard EMEM supplemented with 10% (v/v) fetal bovine serum. A549 (pulmonary adenocarcinoma) cells were cultured in standard F-12K medium supplemented with 10% (v/v) fetal bovine serum.

Patient-derived xenograft (PDX) cell lines licensed from Charles River Laboratories (CRL) were cultured in standard RPMI1640 medium with 25 mM HEPES and L-glutamine (#FG1385, Biochrom, Berlin, Germany), supplemented with 10% (v/v) fetal calf serum (Sigma, Taufkirchen, Germany) and 0.1 mg/ml Gentamycin (Life Technologies, Karlsruhe, Germany).

Lonza primary-like cell line SAEC-1 were cultured using the Lonza SAGM Small Airway Epithelial Cell Growth Medium BulletKit (CC-3118). Lonza Normal Human Bronchial Epithelial (NHBE) and Chronic Obstructive Pulmonary Disease (COPD) primary-like cell lines were cultured using Lonza Bronchial Epithelial Cell Growth Medium BulletKit (CC-3170).

Approximately 24 hours prior to conducting experimentations, cells were plated to achieve a confluence of 70-80% on the day of transfection.

### Transfections

For transient transfections, Lipofectamine 3000 (Thermo Fisher) was used according to the manufacturer's instructions. Briefly, for each well, 100 ng of plasmid DNA was mixed with 0.2 μL of P3000 reagent and 0.2 μL of Lipofectamine 3000 and 2 ng of control DNA in 100 μL Opti-MEM medium and the mixture was incubated at room temperature for 20 minutes. The transfection mixture was then added to the cells in a 96-well plate and the cells were incubated for 24 hours.

### Luciferase Assays and Analysis

Approximately 24 hours after the transfection, firefly luciferase and Renilla luciferase levels were measured from each well using the Promega Dual-Glo Luciferase System (E2940) with a working volume of 50 μL.

Data are presented as raw output of Firefly Luciferase Relative Light Units (FLUC RLUs) relative to constitutively active promoters, % of EF1A or % of CMV or relative to another strong, constitutive promoter. A plasmid encoding for Renilla luciferase was added into transfection mixtures at a low ratio to control for variance in transfection efficiency between parallel wells of cells. Normalization for transfection and well-to-well variability was performed by dividing the FLUC RLU output by the Renilla luciferase (RLUC) RLU output from the CMV-RLUC co-transfection control. Normalized FLUC/RLUC may also be presented as % of expression relative to EF1A.

Chromatin Immunoprecipitation (ChIP)—Quantitative PCR (qPCR)

24 hours after transfection, cells (10-cm dish) were fixed with 1% formaldehyde for 10 minutes at room temperature. Cells were then washed twice with ice-cold PBS. Then, cells were harvested using cell scraper in 2 ml of ice-cold PBS with protease inhibitors and centrifuged at 2000 rpm at 4° C. for 5 minutes. The cell pellets were lysed in 200 μL (per 100 μL cell pellet) of 1% SDS lysis buffer (1% SDS, 10 mM EDTA, 50 mM Tris-HCl, pH 8.1) with protease inhibitors, and the extracts were sonicated using a Misonix Sonicator 3000 instrument and a microtip probe (use 1 second on, 0.5 second pulse for 15 seconds at power setting of 2; put on ice for 15 seconds to chill the tube; 6-9 cycles were performed). Samples were then centrifuged at 12,000×g at 4° C. for 10 minutes, and supernatant was collected. Samples were diluted to 2 ml in ChIP dilution buffer (1% Triton X-100, 2 mM EDTA, 20 mM Tris-HCl, pH 8, 150 mM NaCl) with protease inhibitors. 40 μL of the diluted sample was kept aside as the input fraction before preclearing with non-blocked 75 μL ProteinA Agarose/Salmon Sperm DNA (50% Slurry) for 30 minutes at 4° C. with agitation. Agarose was pelleted by centrifugation (10,000×g-15,000×g) and the supernatant fraction was collected. 60 μL blocked agarose beads were added to the supernatant fraction per reaction with control rabbit IgG, anti-c-Jun, or anti-FRA2 rabbit antibodies (purchased from CellSignaling) and incubated at 4° C. overnight with rotation. Immune complexes were washed once with low salt wash buffer, once with high salt wash buffer, once with LiCl wash buffer with 0.1% SDS, and two times with Tris-EDTA buffer. DNA-protein complex was eluted in ChIP elution buffer (1% SDS, 0.1M NaHCO₃). Cross-links were reversed at 65° C. for 2 hours. DNA was purified by QIAquick Spin Miniprep Kit following the manufacturer's protocol (Qiagen). For all quantitative PCR (qPCR) analyses, Taqman primer/probe assay for target gene promoter binding was performed using QuantStudio 6 Flex machine.

### RNA-Seq and Principal Component Analysis

Briefly, raw sequencing data was aligned to GRCh38/hg38 using Spliced Transcripts Alignment to a Reference (STAR). The resulting Binary Alignment Map (BAM) files were analyzed using feature counts against a transcriptomic reference based on Gencode 36 (https://www.gencodegenes.org/human/release_36.html). The resulting gene-level counts for protein-coding genes were upper-quartile normalized, transformed into Fragments Per Kilobase of transcript per Million mapped reads (FPKM-UQ), and log 2 transformed. Clinical Proteomic Tumor Analysis Consortium (CPTAC) RNA-seq data in FPKM-UQ unit was directly downloaded from linkedOmics data portal.

PCA (R package PCAtools version 2.6.0), a dimensionality reduction method, was used to cluster the samples using the RNA-seq profiles. PCA was either performed on all genes, expression-quantified as FPKM-UQ, or on genes restricted to the relevant gene sets downloaded from MSigDB (https://www.gsea-msigdb.org/gsea/msigdb/).

### Results

### Synthetic Promoters Dependent on Dysregulated FOS and a Core-Cancer Specific Promoter are Highly Active

The use of synthetic promoters composed of tiled transcription factor binding sites (TFBSs) and a minimal core promoter to improve gene expression in cancer cells was investigated. The expression of a reporter gene expressed from a panel of synthetic promoter constructs was tested and the expression levels were compared to the expression levels of the reporter expressed from the endogenous BIRC5 (Survivin) promoter, a combination of three endogenous cancer-activated promoters, or constitutive controls such as EF1α and CMV promoters.

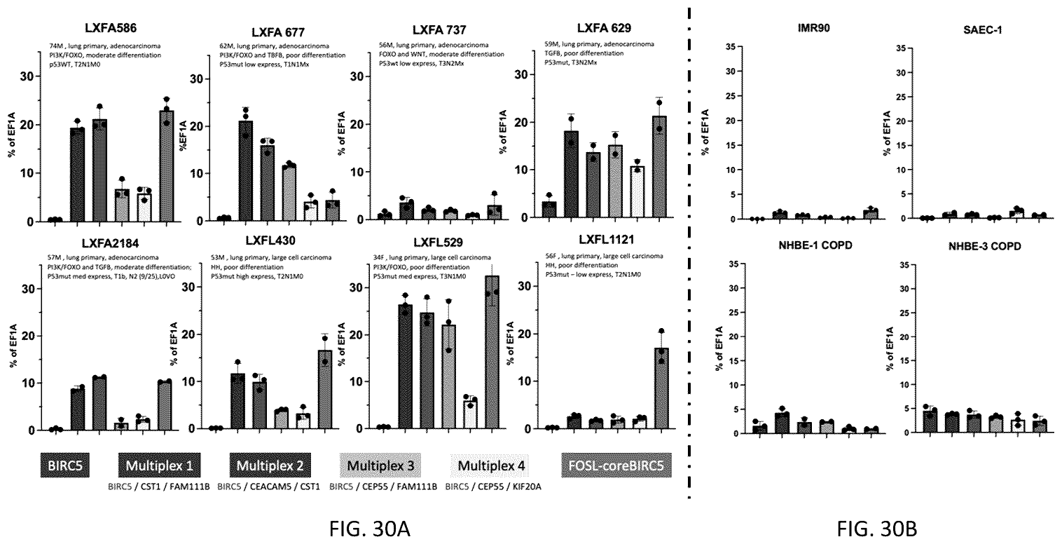

FIG. 30A demonstrates that the synthetic constructs generated (FOS-coreBIRC5) outperformed the individual or multiplexed endogenous promoters in terms of both strength and sensitivity across PDX cell lines, having up to 10-fold more signal than the endogenous BIRC5 (Survivin) promoter and equivalent or better signal than the multiplexed endogenous promoters. The FOS-coreBIRC5 promoter also showed sensitivity capturing patient LXFL1121, which was missed by all other multiplexed endogenous promoters. The FOS-coreBIRC5 promoter had similar expression level as the endogenous BIRC5 promoter in normal lung fibroblast, bronchial epithelial (NHBE), and small airway epithelial cells (SAEC) (FIG. 30B).

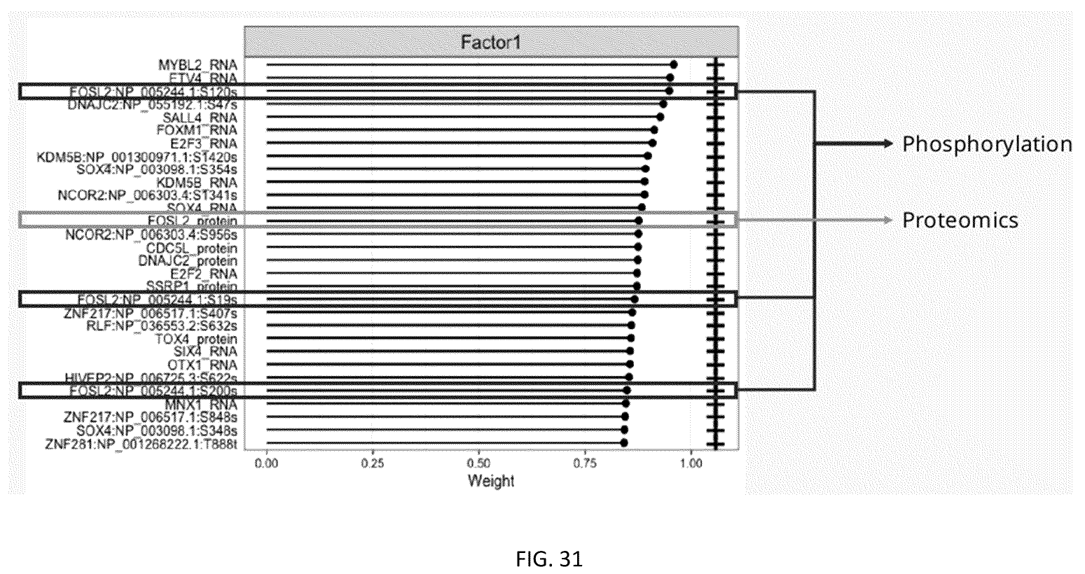

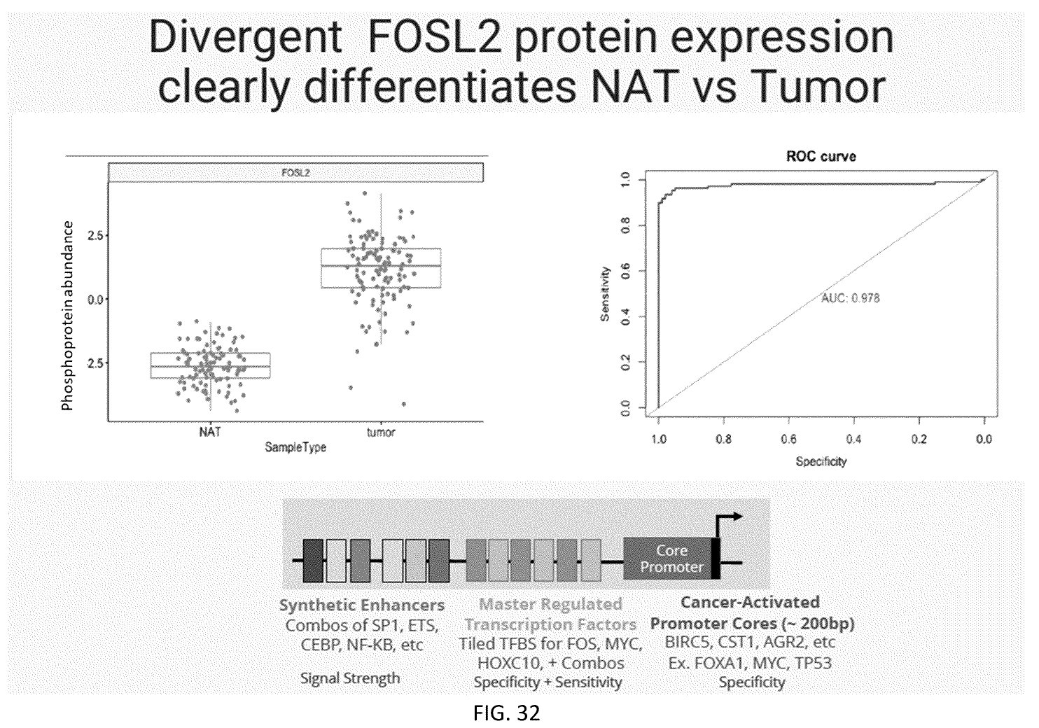

While the FOS binding site used is the DNA binding motif for a variety of bZIP-like transcription factors, including Jun and FOS family (FOS, FOSB, FOSL1, and FOSL2), cancer-activated upregulation of FOSL2 is expected and is primarily driving the differential expression of this promoter, as FOSL2 was identified as one of the top candidates in the multi-omics analysis performed as a part of Multi-Omics Factor Analysis (MOFA) for NSCLC specific transcription factor identification (FIGS. 31-32). This MOFA utilized an unsupervised integration of different-omics data available from CPTAC's LUAD and lung squamous cell carcinoma (LUSQ) tumor and patient matched Normal Adjacent Tissues (NAT) samples and restricted gene analysis to TFs and phosphorylation sites of those TFs. The initial analysis of NSCLC patients consistently showed FOSL2 as one of the top activated transcription factors in NSCLC, especially by protein abundance and phosphorylation abundance (FIGS. 31-32). However, based on the literature evidence, other various FOS family members can be also used, as high FOSL1 expression has been shown in KRAS driven lung and pancreatic cancers, and gross upregulation of c-Fos and its binding partner c-Jun has been shown in NSCLC.

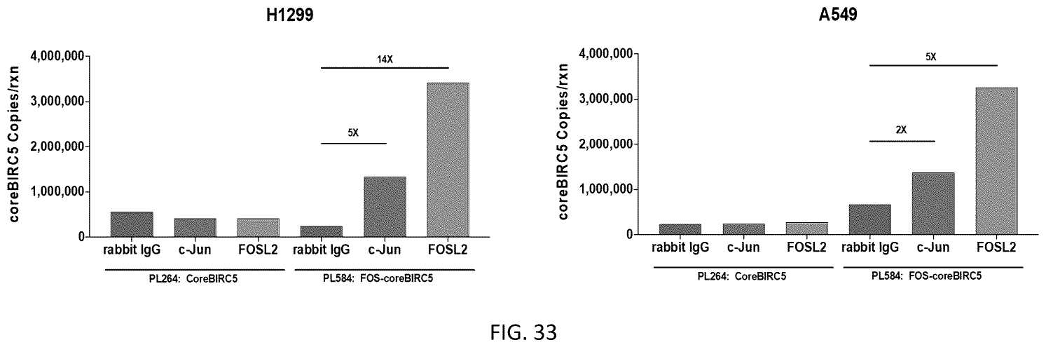

To prove the hypothesis that FOS-coreBIRC5 activity is directly responsive to varying levels of FOSL2, a chromatin immunoprecipitation (ChIP) assay was performed to determine whether the FOSL2 protein binds directly to the FOS-coreBIRC5 in cell lines where the FOS-coreBIRC5 promoter is active. The results showed that the FOS-coreBIRC5 sequence is 14 times more enriched in the FOSL2 pulldown versus the non-specific pulldown of the same construct (FIG. 33). The coreBIRC5 promoter alone construct that does not contain the putative FOSL2 binding sequences serves as a negative control, demonstrating that there is no enrichment of the DNA sequence upon a pulldown of the FOSL2 or c-Jun proteins. This mechanistically proves that the response element binds directly the FOSL2 transcription factor as well as its dimerization partner, c-Jun.

Additional TF Response Element Promoters Using coreBIRC5

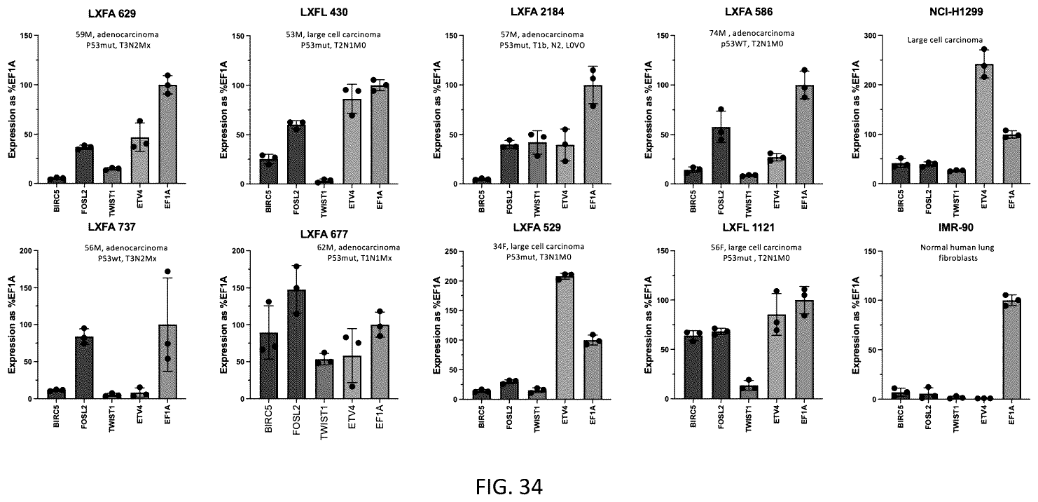

In addition to the FOS response element, more than 20-30 working response elements to transcription factors dysregulated in NSCLC were engineered. A high-throughput screening approach was implemented to test and design thousands of unique response elements at a time. FIG. 34 shows a small subset of these transcription factors (FOSL2, ETV4, TWIST1) across a panel of eight different lung cancer PDX cell lines, as well as NSCLC cell line H1299 and control normal fibroblast cell line IMR-90, demonstrating that several of these chimeric promoters can drive fairly high expression in a variety of cancer cell lines, especially compared to the initial endogenous (1000 bp) BIRC5 promoter, while still maintaining high specificity.

### Predictability of Synthetic Promoters: B-Cat/Wnt Pathway Synthetic Promoter

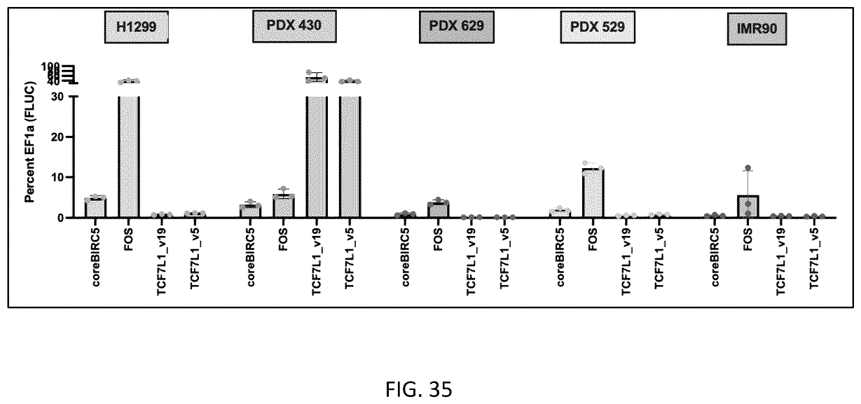

While many of the synthetic TFBS constructs tested had increased sensitivity and specificity relative to endogenous promoters, it was also found that synthetic promoters containing binding sites for the TCF/LEF family of transcription factors showed significant activity in only one of the primary models (PDX430, FIG. 35), while maintaining high specificity as evidenced by a lack of signal in normal cell lines such as IMR-90 fibroblasts. As TCF7 is a well-studied acting transcription factor in the B-catenin/Wnt signaling pathway, it was postulated that this cell line uniquely represented a Wnt-dependent tumor.

A principal component analysis (PCA) was performed on the transcriptome data from Charles River on all NSCLC PDX tumors, as well as CCLE, the Cancer Cell Line Encyclopedia. The primary differentiator (PC1) was driven by inherent transcriptomic differences between the PDX cell lines (blue) and the immortalized traditional cell lines (red), likely due to similar genetic drift in the immortalized cell lines due to many generations of adjustment to plastic. However, by PC2, PDX430 was uniquely situated in PC2, and within the CCLE cell lines, NCI-H520 and LK2 plot similarly by PC2. This is driven by nearly identical profiles in key Wnt pathway genes Wnt7B, CCND1, FZD3, AXIN2, and NKD1.

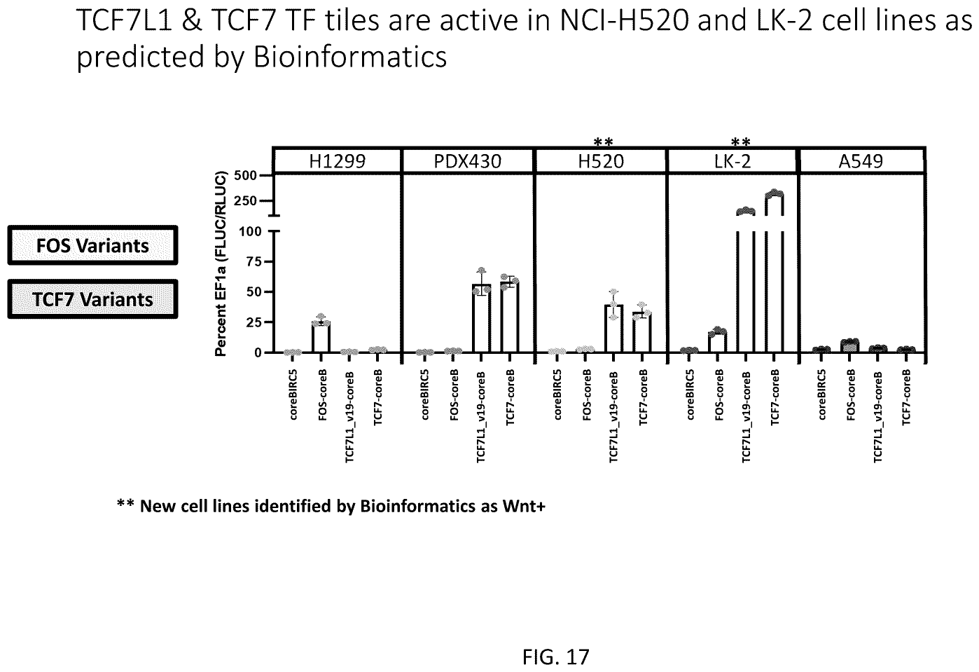

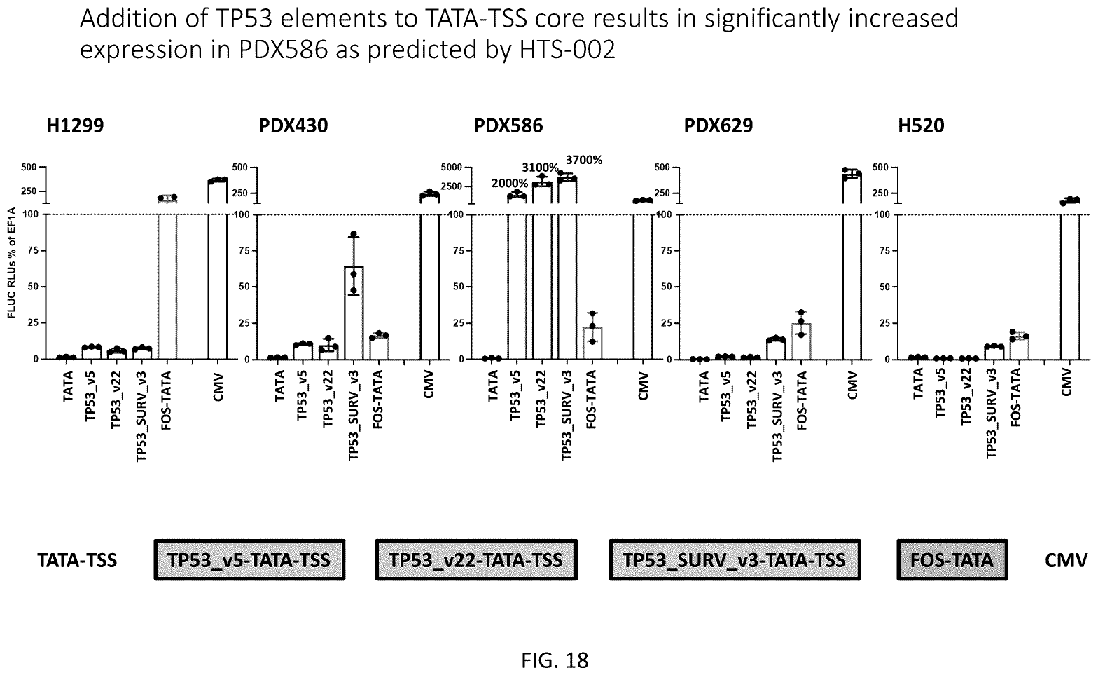

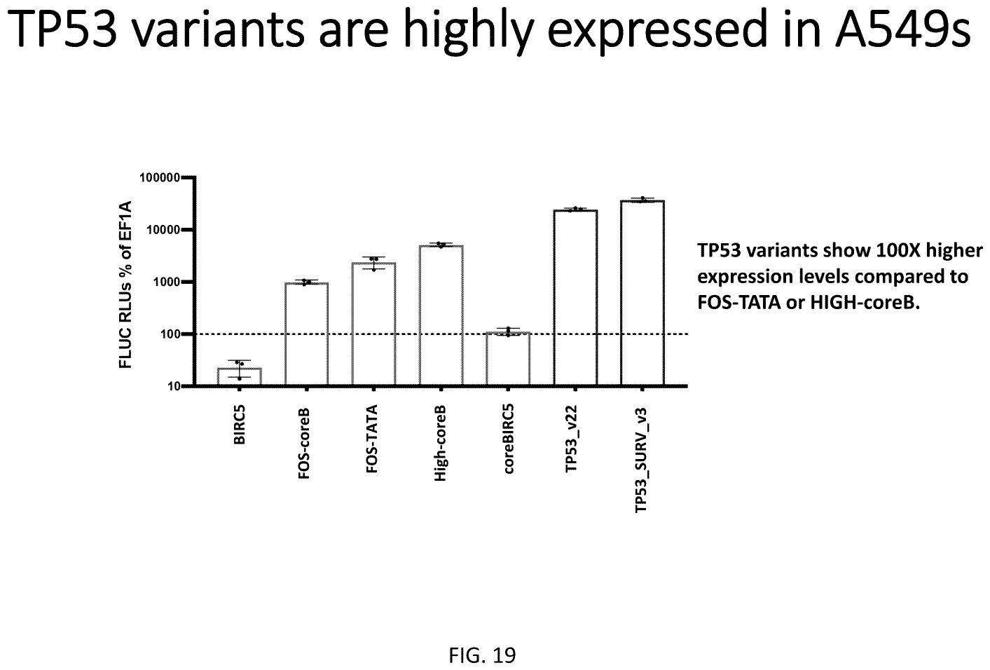

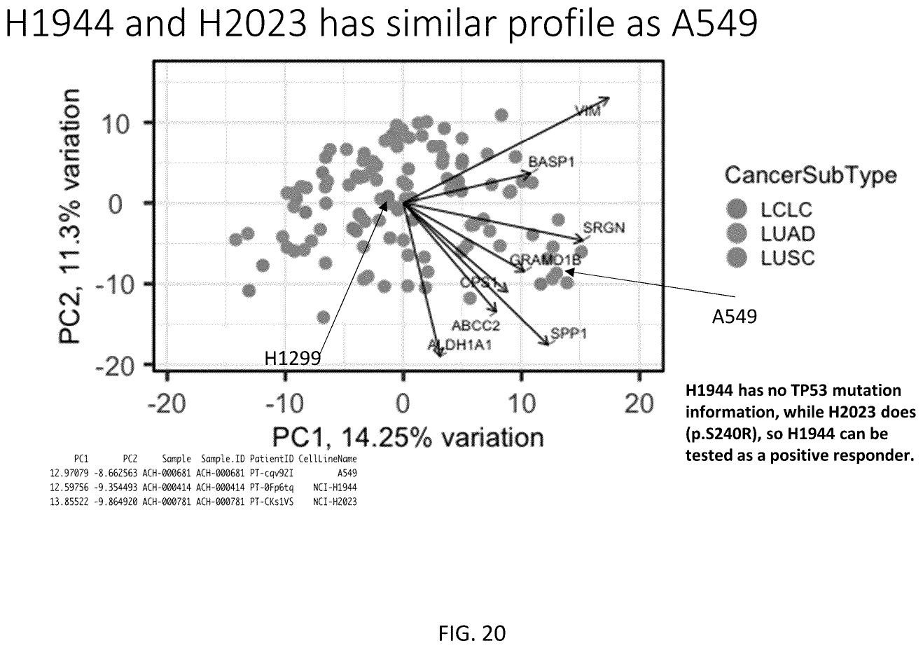

These similarly profiled cell lines were purchased and transfected with a panel of synthetic constructs including the TCF7 and TCF7L1 variants, and as shown in FIG. 17, H520 and LK-2 predictably activated the TCF7 promoter, while KRAS-driven cell lines H1299 and A549 did not show any activation of the Wnt-pathway promoter, especially as compared to the FOS driven promoter.

### Core Promoter Signal Elements

In addition to cancer-specific response elements, synthetic promoters can also be engineered with general activating elements comprising transcriptional factor binding sites and elements, GC-Box, antioxidant response elements (ARE). These can be combined with minimal core promoters or with synthetic promoter constructs containing TFBS such as FOSL-core BIRC5.

The “Low,” “Medium,” and “High” expressing elements were added to core promoters. Addition of activating elements resulted in increased signal strength of the promoters.

### New Cancer-Specific Core Promoters

In addition to modifying proximal promoter regions, alternative core promoters from endogenous promoters beyond BIRC5 can be combined with synthetic enhancer sequences to increase signal strength while maintaining specificity. Based on the analysis of coreBIRC5 element, it was hypothesized that other “core” regions of endogenous cancer-dysregulated promoters could also serve as the core element in the synthetically engineered promoters and it was sought to understand whether they also maintain the specificity driven by coreBIRC5 while increasing sensitivity or signal strength.

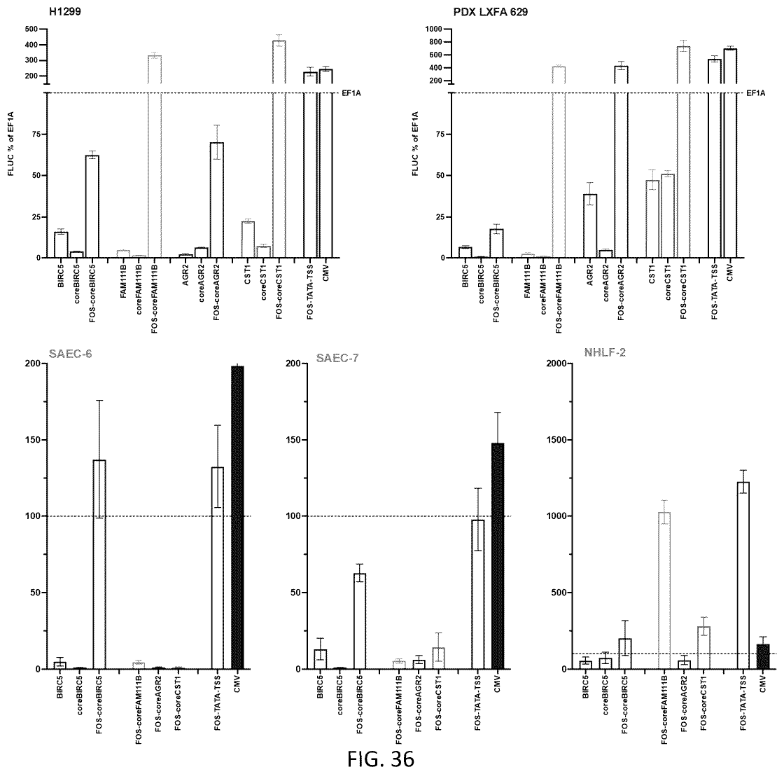

Based on the previous positive results with the FAM111B, AGR2 and CST1 promoters, the use of the core elements isolated from these were first explored. Increasingly short variants of the core were tested and the 165 bp (FAM111B), 360 bp (AGR2), and 191 bp (CST1) version of these cores were further chosen. As shown in FIG. 36, new chimeric promoters FOS-coreFAM111B, FOS-coreAGR2, FOS-coreCST1 led to dramatic improvements in signal strength (up to 20-fold) as compared to FOS-coreBIRC5. As previously suggested, these constructs had improvements over the full-length version of the respective endogenous promoters as well. The new cores also maintained high specificity compared to the completely permissive core TATA-TSS (gray) in normal lung models of human small airway epithelial cells (SAEC-6, SAEC-7) and normal human lung fibroblasts (NHLF-2), although core-FAM111B may not maintain as much specificity in fibroblasts.

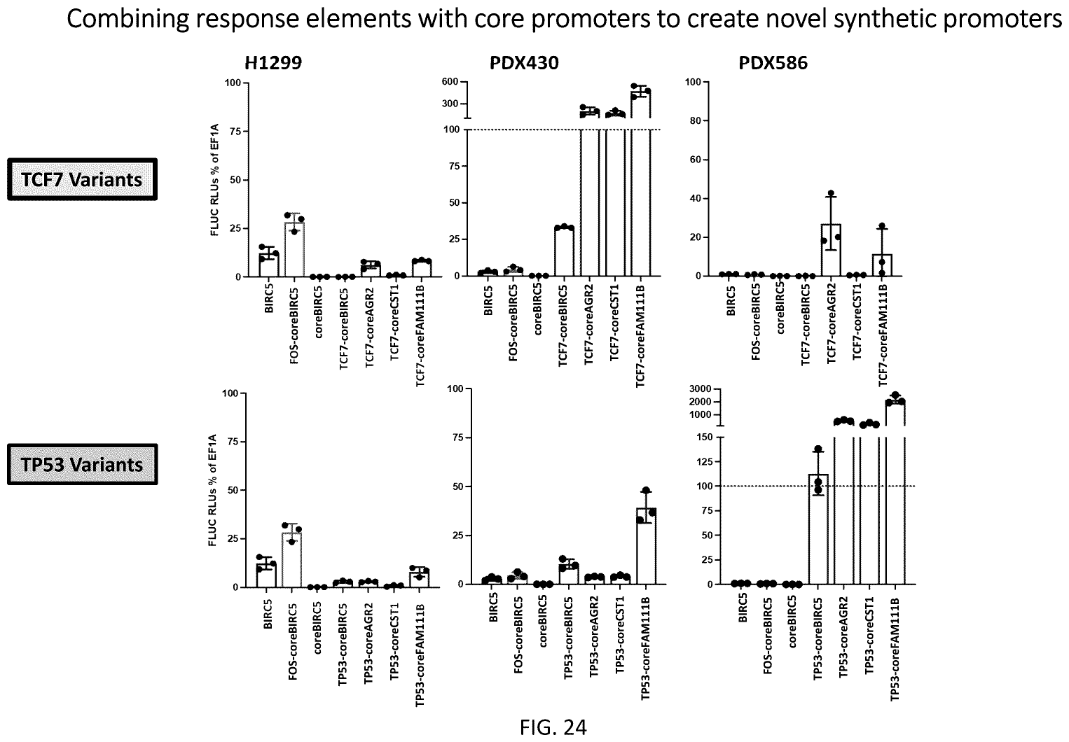

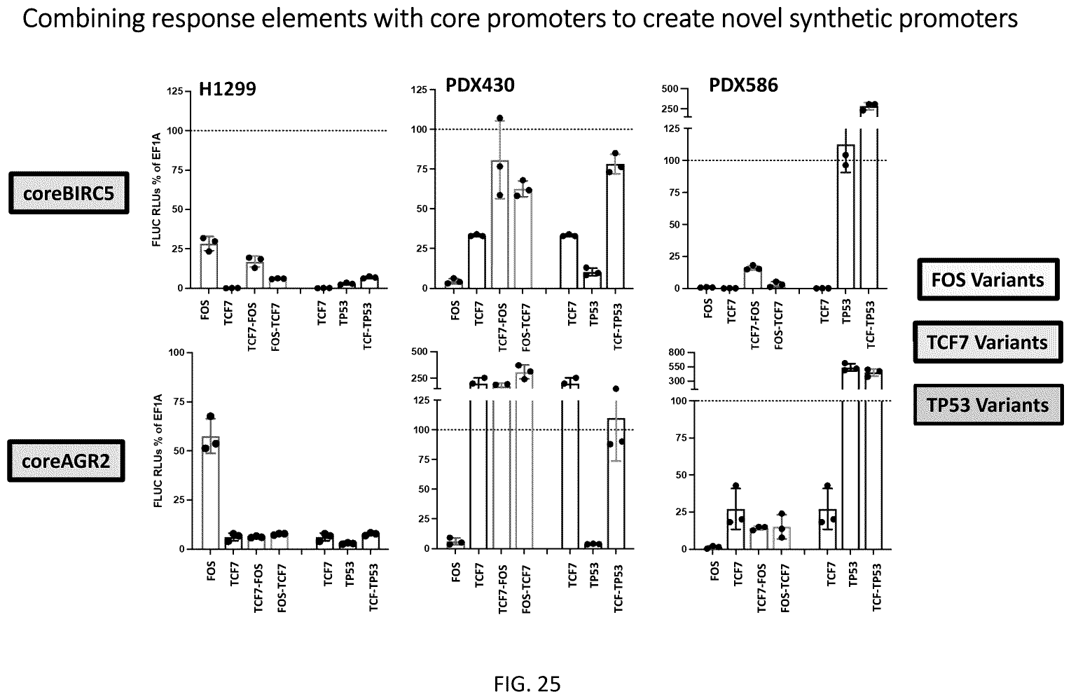

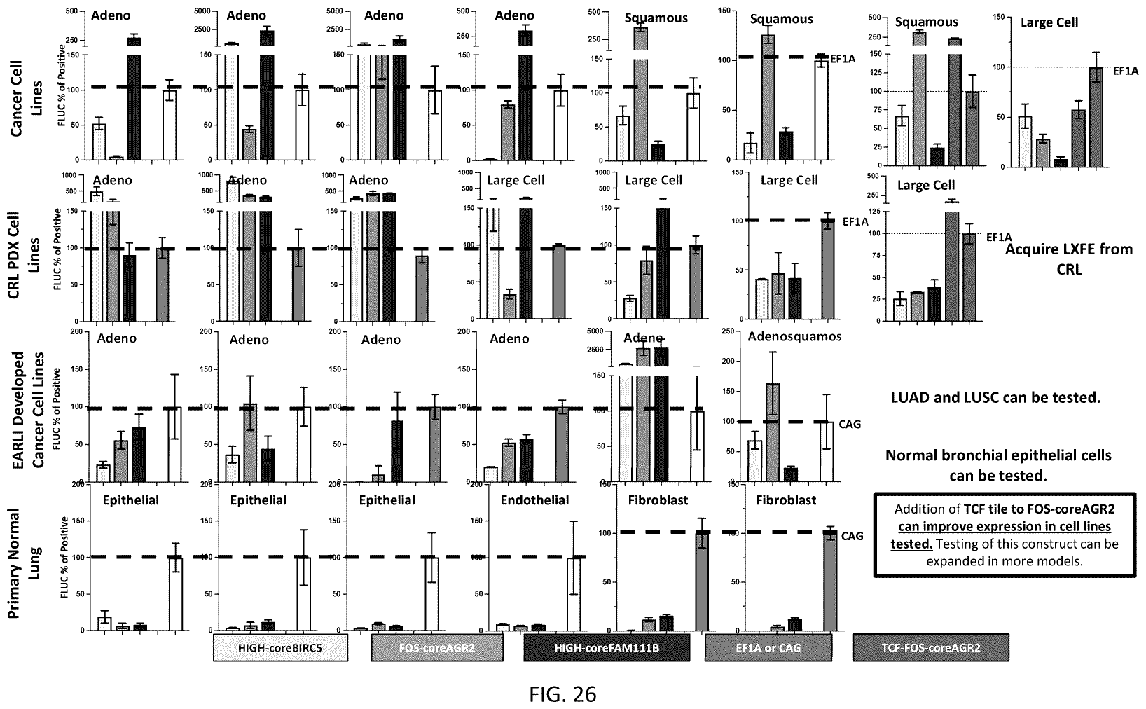

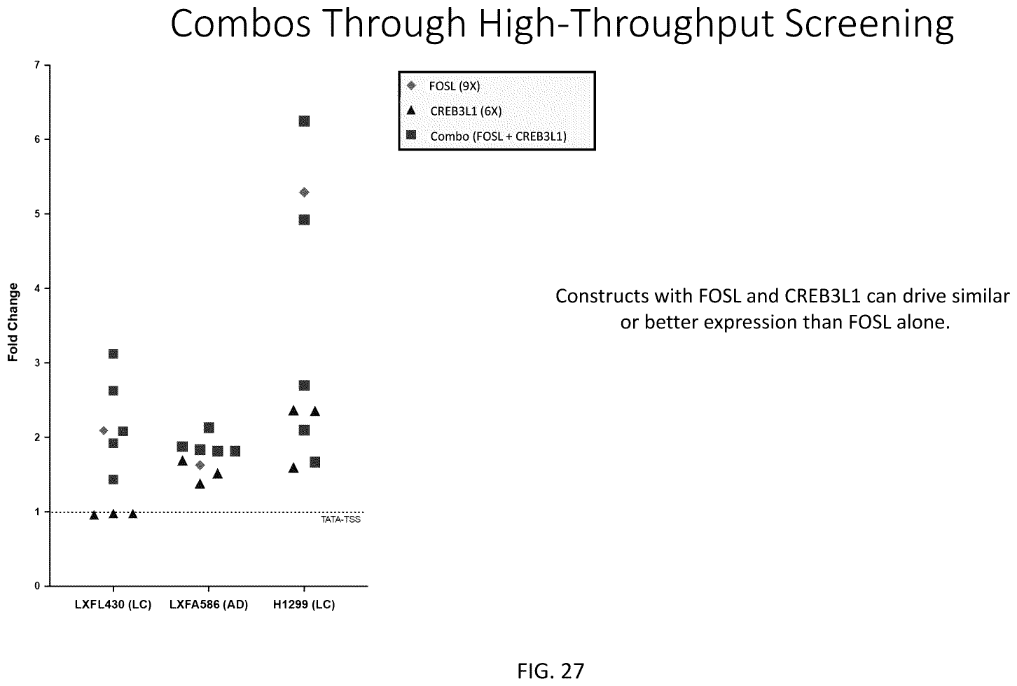

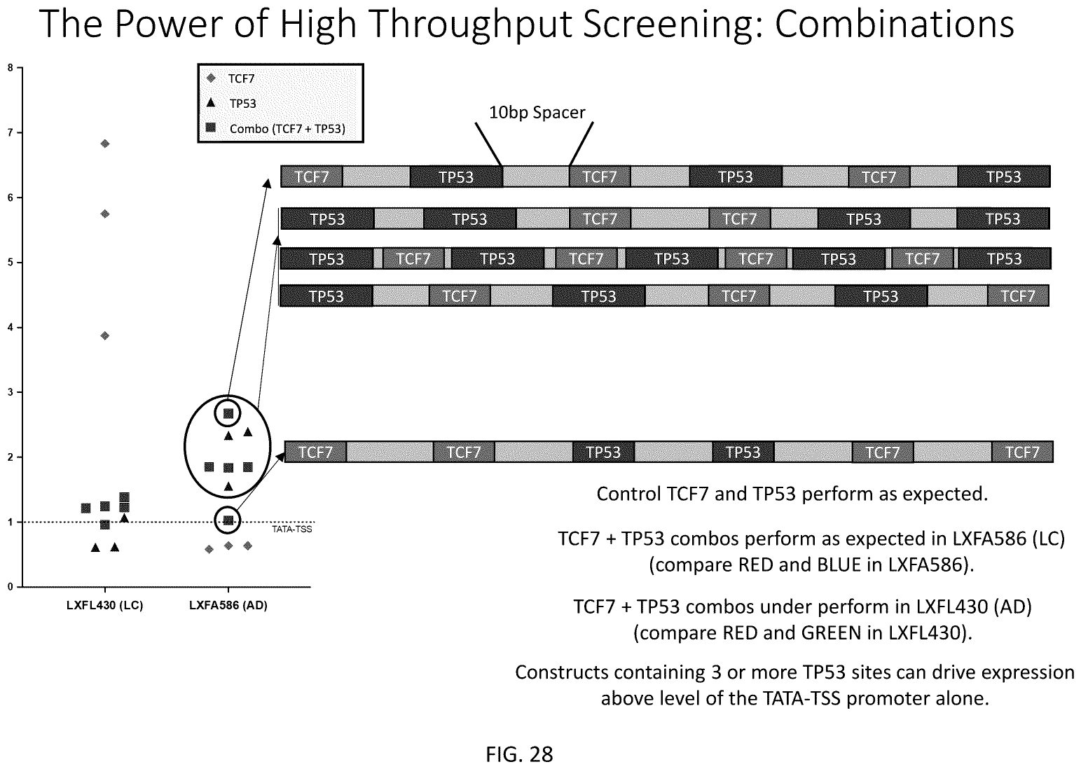

Additional experiments have similarly shown that alternative core promoters coreAGR2 and coreCST1 can partner well with TFs besides FOS to drive higher signal while maintaining cancer specificity (FIGS. 24-26). FIG. 24 shows that response elements for TCF7 and TP53 which are particularly active in cell lines PDX430 and PDX586, respectively, gained additional strength without loss in specificity by using alternate core promoters AGR2, CST1 and FAM111B. Furthermore, addition of TCF tiles to FOS-coreAGR2 improved expression of the reporter gene in various cell lines tested, including cancer cell lines, CRL PDX cell lines, and primary normal lung cells (FIG. 26).

### Conclusion

By creating synthetic response elements that are bound by the presence of transcription factors whose expression is dysregulated in cancer, chimeric promoters with high sensitivity and specificity have been engineered to drive cancer specific expression of a reporter gene or a gene of interest. Engineered synthetic promoters can drive substantially higher expression of a reporter gene or a gene of interest than the endogenous promoter of the BIRC5 gene. Furthermore, synthetic promoters can maintain cancer specificity when comparing lung cancer models to normal small airway epithelial cells or lung fibroblasts. Most importantly, the activation of synthetic promoters as opposed to endogenous promoters is highly predictable, as demonstrated by the analysis of the TCF7 chimeric promoter.

### Example 3: Detection of Hepatocellular Carcinoma in an Orthotopic Mouse Model

Synthetic promoters designed for highly specific cancer-activated expression of a gene in tumors is applicable to malignancies beyond the non-small cell lung cancer (NSCLC). In this example, the utility of a rational-based sequence engineered approach of a highly specific and strong liver cancer promoter is demonstrated. For example, a known alpha-fetoprotein (AFP) promoter drove the expression of a gene up to 200-fold higher in liver cancer cell lines without any increase in basal activity in non-liver and normal cell lines. The promoter-mediated strong cancer-activated expression, when combined with the reporter and delivery aspects of the platform, was demonstrated by blood-based biomarkers and imaging markers (assayed by staining) in an in vivo model of liver cancer.

Hepatocellular carcinoma can greatly benefit from additional technologies in the early detection and diagnostic space. Risk of HCC is highly elevated in patients with chronic liver disease, including those with chronic Hepatitis B (HBV) or with cirrhosis from other severe liver diseases such as HBV, HCV, or NASH. At-risk patients are closely monitored for disease progression into a malignancy, but the tools currently available are highly limited. Semi-annual abdominal ultrasounds and the AFP blood marker test are the only two surveillance tests in clinical guidelines and with broad adoption, but their performance has been quite poor in detecting early-stage malignancies, which are much more likely to be cured & treated effectively than later stage cancers.

Both abdominal ultrasound and AFP blood tests have less than optimal sensitivities, with the AFP test shown to detect HCC with only 63% sensitivity. In particular, ultrasound effectiveness is highly variable based on operator, and is markedly difficult in obese patients and patients with NASH. A novel diagnostic modality described herein could bridge the gap between these screens and diagnosis, either bypassing physical biopsies or further reducing the population that is subjected to them. These patients include those for whom ultrasounds can be inconclusive due to high levels of cirrhosis or indeterminate liver nodules that simply don't have the hallmark radiological features of HCC. Additionally, for patients with small liver nodules (<2 cm), it is difficult to distinguish HCC from benign dysplastic nodules or intrahepatic cholangiocarcinoma (bile duct cancer).

From a scientific perspective, lipid nanoparticles (LNPs) have traditionally been known for their ability to mediate highly effective delivery in the liver, which can be a benefit to liver cancer diagnostics platform, provided that the reporter expression post-delivery is still highly cancer-specific to avoid noise from normal liver. This example provides a strong example of a rational engineering approach applied to endogenous promoters to create a unique liver cancer promoter (named AFP-3) and show that when coupled with a LNP formulation, the platform can provide strong cancer-activated synthetic biomarker expression in primary liver tumors.

The goal is to assess the signal-to-noise response of a liver-tropic formulation using an engineered promoter specific to liver cancer in the Hep3B orthotopic liver tumor model in mice.

### Engineering & Testing of the AFP-3 Promoter

### Cloning

To generate a reporter construct for use in measuring promoter activity, DNA fragments of interest were cloned into a standard Firefly Luciferase (FLuc) reporter vector from Promega (pGL4.10[luc2] Promega E6651) using the KpnI and NheI restriction enzymes.

The promoter region of interest was amplified using PCR primers with flanking restriction enzyme sites, and the PCR product was purified and digested with the appropriate restriction enzymes. BIRC5 promoter was amplified from approximately −1000 bp to +33 bp relative to the predicted transcriptional start site (TSS) of the endogenous promoter. The AFP promoter was amplified from approximately −250 bp to +28 bp relative to the TSS. AFP-3 was subcloned from AFP using mutagenic primers containing the desired point mutations. Ligated vectors were transformed into E. coli Stable cells, and clones were screened by DNA sequencing to confirm the correct assembly.

DNA was scaled up and purified using QIAGEN Plasmid Plus Midi (Cat #12945) or equivalent. Purified DNA was used for subsequent in vitro and in vivo transfections. Promoters were transferred into Nanoplasmid vectors utilizing restriction enzyme cloning with restriction enzymes flanking the promoter region.

### Cell Culture & Transfections

Cells were maintained according to standard protocols with recommended media listed below and incubated at 37° C. and 5% CO₂.

SNU-449, H1299 cells were cultured in standard RPMI1640 medium supplemented with 10% (v/v) fetal bovine serum. HepG2 (human hepatocellular carcinoma), Hep3B (human hepatocellular adenocarcinoma), PLC/PRF/5 (human hepatocellular carcinoma), C3A (clonal derivative of HepG2), MRC-9 (fibroblast) and IMR-90 (control normal fibroblast cell line) cells were cultured in standard EMEM supplemented with 10% (v/v) fetal bovine serum. MeWo (human melanoma cell line) cells were cultured in standard DMEM supplemented with 10% (v/v) fetal bovine serum.

Approximately 24 hours prior to transfections, cells were plated to achieve a confluence of 70-80% on the day of transfections. For transient transfections, Lipofectamine 3000 was used according to the manufacturer's instructions. Briefly, for each well, 100 ng of plasmid DNA was mixed with 0.2 μL of P3000 reagent and 0.2 μL of Lipofectamine 3000 and 2 ng of control DNA in 100 μL Opti-MEM medium and the mixture was incubated at room temperature for 20 minutes. The transfection mixture was added to the cells in a 96-well plate and incubated for 24 hours.

### Luciferase Readouts

Approximately 24 hours after transfection, firefly luciferase and renilla luciferase levels were measured from each well using the Promega Dual-Glo Luciferase System (E2940) with a working volume of 50 μL.

### Hep3B Murine Experiment

### Cell Culture

The Hep3B-luc tumor cells (ATCC, Manassas, VA, cat #HB-8064) were maintained in vitro as a monolayer culture in EMEM medium supplemented with 10% fetal bovine serum, 100 U/mL penicillin and 100 μg/mL streptomycin, at 37° C. in an atmosphere of 5% CO₂ in air. The tumor cells were routinely sub-cultured twice weekly by trypsin-EDTA treatment. The cells growing in an exponential growth phase were harvested and counted for tumor inoculation.

### Orthotopic Tumor Implantation

The female BALB/c nude mice were anesthetized with 20 L/g Avertin (2,2,2-tribromoethanol). For pain relief, the animals were dosed with 10 mg/kg of Carprofen 30 minutes before surgery and 6 hours post-surgery.

Each of the anesthetized mice was properly positioned. The abdomen skin was sterilized with 70% ethanol and the surgical site was prepared in a sterile condition. A small incision was across the abdominal wall. The left lobe of the liver was identified and exposed. Approximately 3×10⁶ Hep3B-luc cells with BD Matrigel in 20 μL (PBS: Matrigel=1:1) were injected into the left lobe of the liver. The injection site was monitored for leakage of cells and after confirmation of no leakage of cells, the left lobe of the liver was placed back to the abdominal cavity. The abdominal wall was then closed, and the skin was closed with surgical suture. These mice were continuously monitored for their complete recovery from anesthesia.

### Bioluminescence Measurements

The surgically inoculated mice were weighted and intraperitoneally injected luciferin at 150 mg/kg. After 10 minutes of the luciferin administration, the animals were pre-anesthetized with the mixture gas of oxygen and isoflurane. When the animals were in a complete anesthetic state, they were moved into the imaging chamber for bioluminescence measurements with IVIS (Lumina III). The bioluminescence of the whole animal body, including primary and metastatic tumors, was measured and images were recorded.

### Assignment to Groups

Bioluminescence from the Hep3B-luc tumor cells were measured on all tumor bearing mice at Day 7, Day 14, and Day 20 post implantation. Randomization of animals for tumor bearing mice was based on the imaging at Day 20 post implantation, and randomization of non-tumor bearing mice was based on the body weight taken at Day 20 post implantation. Mice were selected at Day 21 post implantation, and mice bearing established tumors were assigned to 9 groups (1, 4, or 5 mice/group) using an Excel-based randomization procedure performing stratified randomization based upon the intensity of bioluminescence. Normal mice (no tumors) were also assigned to 5 groups (2 or 5 mice/group) using the same method. Administration of test article was started at Day 21 post implantation.

### Observations

All the procedures related to animal handling, care and the treatment in the study were performed according to the guidelines approved by the Institutional Animal Care and Use Committee (IACUC) of WuXi AppTec following the guidance of the Association for Assessment and Accreditation of Laboratory Animal Care (AAALAC). At the time of routine monitoring, the animals were daily checked for any effects of tumor growth and treatments on normal behavior such as mobility, food and water consumption (by looking only), body weight gain/loss (body weights were measured twice a week and at Day 20 post implantation as well as every occurrence prior to bleed), eye/hair matting and any other abnormal effect as stated in the protocol. Death and observed clinical signs were recorded on the basis of the numbers of animals within each subset.

### Sample Collection and Endpoints

### Serum Collection:

For Groups 1, 2, 9, 13 and 14: Bleed 1 day before testing of test article, and at 48 hours after dosing (terminal).

### Tissue Collection:

For all non-tumored mice Groups 3-14: collect left lobe and right lobe separately and snap frozen at 48 hours after dosing.

For all tumored-mice Groups 3-13: collect tumor, left lobe and right lobe separately, bisect each of them and snap frozen half, then the other half into FFPE at 48 hours after dosing.

### Animals & Housing Conditions

Species: Mus musculus
Strain: BALB/c nude
Age: 6-8 weeks
Sex: female
Body weight: 18-22 g
Number of animals: 56 mice plus spare
Animal supplier: Beijing Vital River Laboratory Animal Co. LTD
Animal quality certificate number: 20221208Abzz0619000836, 20221208Abzz0619000874, 20221212Abzz0619000183

### Housing Condition

The mice were kept in individual ventilation cages at constant temperature (20-26° C.) and humidity (40-70%). Cages were made of polycarbonate with a size of 375 mm×215 mm×180 mm. The bedding material was corn cob, which was changed twice per week. Animals had free access to irradiation sterilized dry granule food during the entire study period. Animals had free access to sterile drinking water.

### Results

### Design and Validation of AFP-3 Promoter for Activation in Liver Cancer

The alpha-fetoprotein (AFP) promoter has been extensively studied and shown to confer selective expression of transgenes in hepatocellular carcinoma (HCC) in vitro and in vivo. The AFP transcript is normally expressed in normal fetal livers but not adult livers, and then is known to be re-activated in about 70% of liver cancers. Thus, circulating AFP protein is a well-known marker for liver cancer, but the promoter is also well studied to drive specific expression in liver cancer models proportional to the level of AFP expression in the HCC studied.

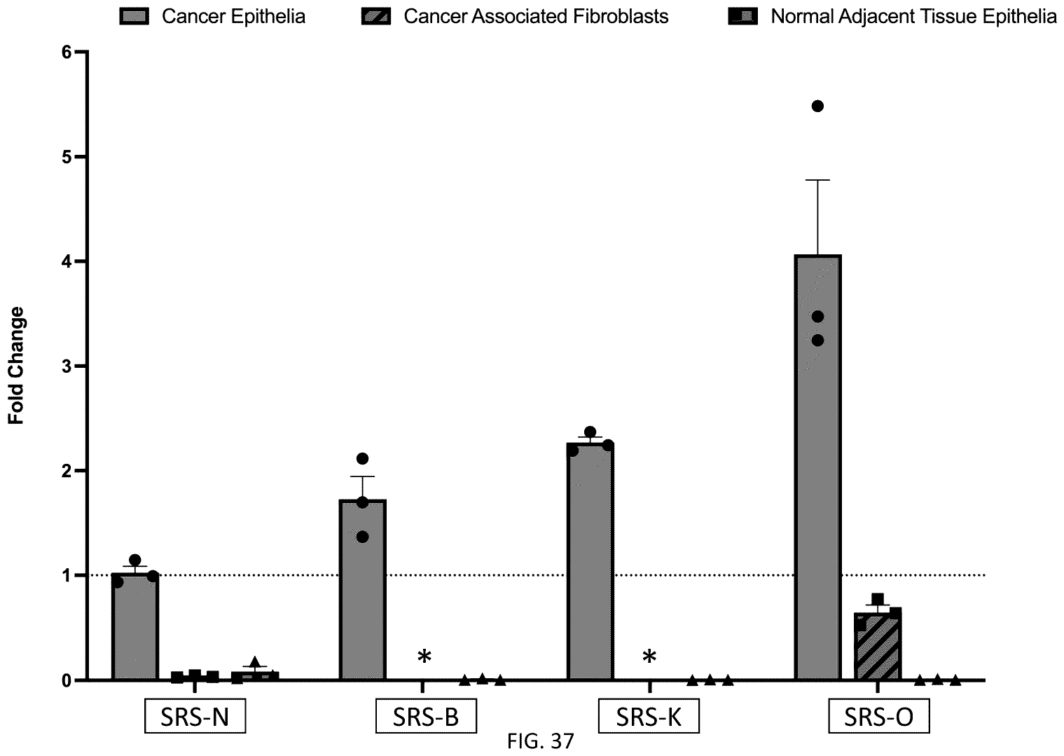

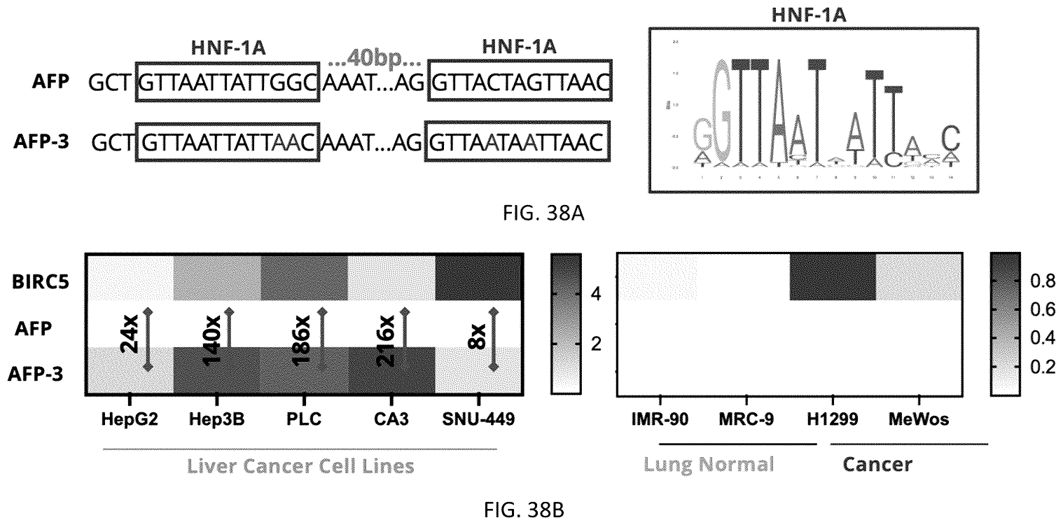

However, as with most endogenous promoters, the level of expression from the AFP promoter is remarkably low, gating its effectiveness in previous applications of liver activated expression. In an effort to create a stronger and more robust activating promoter, a bioinformatic analysis was performed and it was found that there were suboptimal binding sequences for TFs. To boost transcription level, the promoter was rationally engineered by strengthening the dimerized binding sites for HNF-1A, TF binding sites within the AFP promoter, to be closer to the known consensus site for HNF-1A from other promoters (FIG. 38A). Modification of these sequences to have a greater consensus with the ideal binding site can create a more durable and longer interaction of the HNF1A with the AFP promoter, allowing this TF to drive more expression from the TSS in the promoter. These small, rational edits to the base pairs in the promoter led to the reporter construct expressing firefly luciferase to increase expression between 20 to 200-fold in liver cancer cell lines HepG2, Hep3B, PLC, CA3 and SNU-449 (FIG. 38B) while continuing to maintain highly specific liver expression, as shown by continued lack of activity in lung normal cell lines IMR-90, MRC-9, as well as lung cancer H1299 and melanoma MeWo cell lines.

### In Vivo Experimental Design and Groups

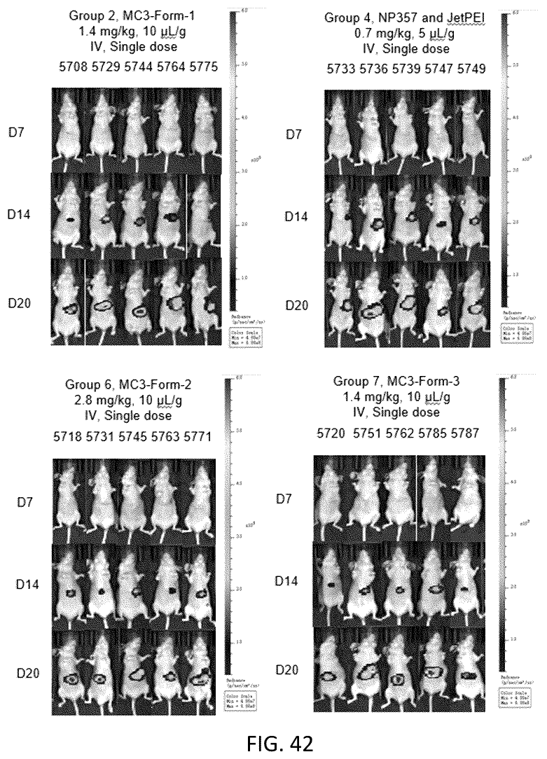

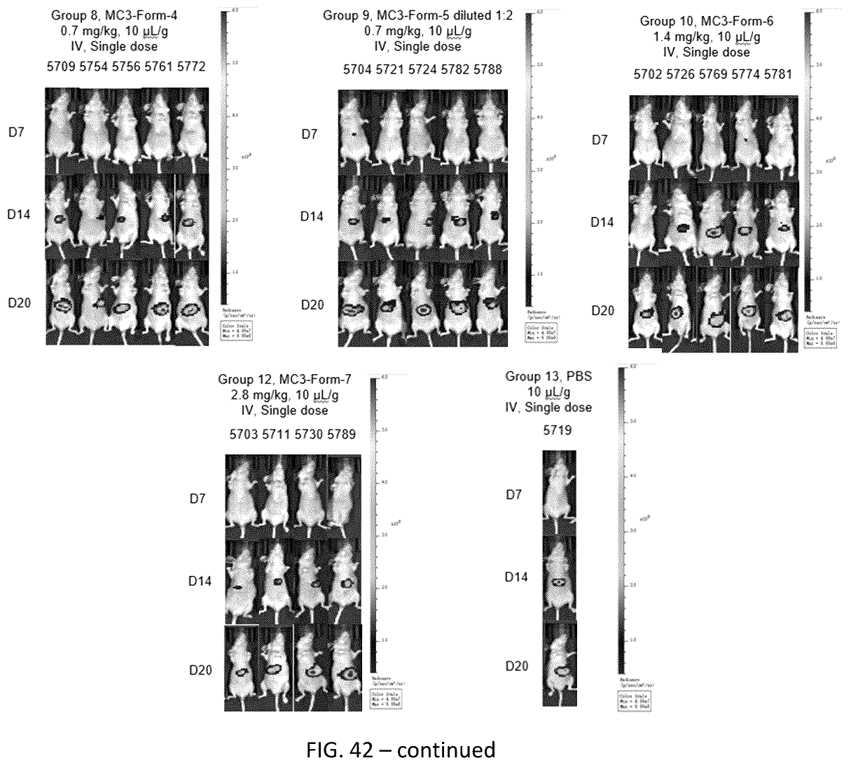

In orthotopic models of HCC, cancer cells are directly inoculated into the liver parenchyma, which allows the tumor to be studied within the correct target organ. In this study, the Hep3B human HCC cell line was orthotopically implanted into the left lobe of the liver for tumor-bearing mice. The cell line used includes a luciferase-based marker to track tumor growth over time and allow for fair assignment of groups based on tumor size. Luciferase and body weight data are shown in Tables 3 & 4 and FIG. 42, demonstrating appropriate tumor growth over 20 days before the mice were randomized and assigned experimental groups in Table 5.

TABLE 3
Raw Data of Body Weight Measurements

| BW | Tumor | Animal No. | 0^{a} | 2 |
|---|---|---|---|---|
| | N | 5797 | 23.36 | 21.05 |
| Group 1 | | 5798 | 23.66 | 20.96 |
| MC3-Form-1 | | 5800 | 21.02 | 19.67 |
| 1.4 mg/kg | | | | |
| 10 μL/g | | 5801 | 22.90 | 20.54 |
| IV, Single dose | | 5806 | 24.14 | 22.89 |
| | | Mean | 23.02 | 21.02 |
| | | SEM | 0.54 | 0.53 |
| Group 2 | Y | 5708 | 23.41 | 20.87 |
| MC3-Form-1 | | 5729 | 20.85 | 18.99 |
| 1.4 mg/kg | | 5744 | 23.32 | 21.01 |
| 10 μL/g | | 5764 | 20.32 | 17.89 |
| IV, Single dose | | 5775 | 20.62 | 18.03 |
| | | Mean | 21.70 | 19.36 |
| | | SEM | 0.68 | 0.67 |
| Group 3 | | 5795 | 23.02 | 21.48 |
| NP357 and JetPEI | | | | |
| 0.7 mg/kg | N | 5805 | 23.02 | 21.48 |
| 5 μL/g | | | | |
| IV, Single dose | | | | |
| | | Mean | 23.02 | 21.48 |
| | | SEM | 0.00 | 0.00 |
| Group 4 | Y | 5733 | 20.97 | 20.76 |
| NP357 and JetPEI | | 5736 | 22.32 | 20.81 |
| 0.7 mg/kg | | 5739 | 20.13 | 17.84 |
| 5 μL/g | | 5747 | 24.00 | 21.31 |
| IV, Single dose | | 5749 | 21.53 | 19.84 |
| | | Mean | 21.79 | 20.11 |
| | | SEM | 0.66 | 0.62 |
| Group 5 | N | 5799 | 23.39 | 21.09 |
| MC3-Form-2 | | | | |
| 2.8 mg/kg | | 5804 | 22.26 | 20.55 |
| 10 μL/g | | | | |
| IV, Single dose | | | | |
| | | Mean | 22.83 | 20.82 |
| | | SEM | 0.57 | 0.27 |
| Group 6 | Y | 5718 | 21.20 | 17.81 |
| MC3-Form-2 | | 5731 | 23.74 | 19.57 |
| 2.8 mg/kg | | 5745 | 23.42 | 18.67 |
| 10 μL/g | | 5763 | 22.43 | 16.96 |
| IV, Single dose | | 5771 | 23.17 | 18.88 |
| | | Mean | 22.79 | 18.38 |
| | | SEM | 0.45 | 0.45 |
| Group 7 | | 5720 | 24.82 | 22.41 |
| MC3-Form-3 | Y | 5751 | 22.02 | 19.09 |
| 1.4 mg/kg | | 5762 | 22.42 | 20.10 |
| 10 μL/g | | 5785 | 22.04 | 19.55 |
| IV, Single dose | | 5787 | 22.59 | 20.40 |
| | | Mean | 22.78 | 20.31 |
| | | SEM | 0.52 | 0.57 |
| Group 8 | Y | 5709 | 22.56 | 19.84 |
| MC3-Form-4 | | 5754 | 22.20 | 20.64 |
| 0.7 mg/kg | | 5756 | 22.45 | 20.25 |
| 10 μL/g | | 5761 | 22.28 | 20.39 |
| IV, Single dose | | 5772 | 23.92 | 20.73 |
| | | Mean | 22.68 | 20.37 |
| | | SEM | 0.32 | 0.16 |
| Group 9 | Y | 5704 | 23.30 | 20.68 |
| MC3-Form-5 diluted 1:2 | | 5721 | 22.65 | 20.57 |
| 0.7 mg/kg | | 5724 | 24.74 | 22.36 |
| 10 μL/g | | 5782 | 21.96 | 19.42 |
| IV, Single dose | | 5788 | 20.09 | 18.21 |
| | | Mean | 22.55 | 20.25 |
| | | SEM | 0.77 | 0.69 |
| Group 10 | Y | 5702 | 21.86 | 18.23 |
| MC3-Form-6 | | 5726 | 23.15 | 19.10 |
| 1.4 mg/kg | | 5769 | 22.05 | 17.21 |
| 10 μL/g | | 5774 | 20.91 | 17.19 |
| IV, Single dose | | 5781 | 22.84 | 18.99 |
| | | Mean | 22.16 | 18.14 |
| | | SEM | 0.39 | 0.41 |
| Group 11 | N | 5794 | 23.76 | 21.79 |
| MC3-Form-7 | | | | |
| 2.8 mg/kg | | 5802 | 22.40 | 19.66 |
| 10 μL/g | | | | |
| IV, Single dose | | | | |
| | | Mean | 23.08 | 20.73 |
| | | SEM | 0.68 | 1.07 |
| Group 12 | Y | 5703 | 25.38 | 22.75 |
| MC3-Form-7 | | | | |
| 2.8 mg/kg | | 5711 | 22.00 | 20.73 |
| 10 μL/g | | 5730 | 21.71 | 19.26 |
| IV, Single dose | | 5789 | 20.93 | 18.48 |
| | | Mean | 22.51 | 20.31 |
| | | SEM | 0.98 | 0.94 |
| Group 13 | Y | | | |
| PBS | | | | |
| 10 μL/g | | 5719 | 22.11 | 21.66 |
| IV, Single dose | | | | |
| | | Mean | 22.11 | 21.66 |
| | | SEM | — | — |
| Group 14 | N | 5791 | 27.22 | 25.08 |
| MC3-Form-5 diluted 1:2 | | 5792 | 21.17 | 19.75 |
| 0.7 mg/kg | | 5793 | 21.84 | 19.94 |
| 10 μL/g | | 5796 | 23.19 | 21.27 |
| IV, Single dose | | 5803 | 21.79 | 20.53 |
| | | Mean | 23.04 | 21.31 |
| | | SEM | 1.10 | 0.98 |

Note:
^{a}days after the start of treatment.

TABLE 4
Bioluminescence

| TV | Tumor | Animal No. | 0^{a} |
|---|---|---|---|
| Group 2 | Y | 5708 | 3.367E+09 |
| MC3-Form-1 | | 5729 | 7.370E+09 |
| 1.4 mg/kg | | 5744 | 8.847E+09 |
| 10 μL/g | | 5764 | 7.500E+09 |
| IV, Single dose | | 5775 | 4.111E+09 |
| | | Mean | 6.239E+09 |
| | | SEM | 1.059E+09 |
| Group 4 | Y | 5733 | 4.683E+09 |
| NP357 and JetPEI | | 5736 | 9.999E+09 |
| 0.7 mg/kg | | 5739 | 8.016E+09 |
| 5 μL/g | | 5747 | 2.125E+09 |
| IV, Single dose | | 5749 | 6.586E+09 |
| | | Mean | 6.282E+09 |
| | | SEM | 1.356E+09 |
| Group 6 | Y | 5718 | 7.971E+09 |
| MC3-Form-2 | | 5731 | 4.694E+09 |
| 2.8 mg/kg | | 5745 | 6.386E+09 |
| 10 μL/g | | 5763 | 2.822E+09 |
| IV, Single dose | | 5771 | 9.288E+09 |
| | | Mean | 6.232E+09 |
| | | SEM | 1.148E+09 |
| Group 7 | Y | 5720 | 3.778E+09 |
| MC3-Form-3 | | 5751 | 8.746E+09 |
| 1.4 mg/kg | | 5762 | 6.683E+09 |
| 10 μL/g | | 5785 | 9.662E+09 |
| IV, Single dose | | 5787 | 2.267E+09 |
| | | Mean | 6.227E+09 |
| | | SEM | 1.415E+09 |
| Group 8 | Y | 5709 | 9.165E+09 |
| MC3-Form-4 | | 5754 | 2.435E+09 |
| 0.7 mg/kg | | 5756 | 4.592E+09 |
| 10 μL/g | | 5761 | 7.135E+09 |
| IV, Single dose | | 5772 | 7.896E+09 |
| | | Mean | 6.245E+09 |
| | | SEM | 1.210E+09 |
| Group 9 | Y | 5704 | 8.262E+09 |
| MC3-Form-5 diluted 1:2 | | 5721 | 3.337E+09 |
| 0.7 mg/kg | | 5724 | 8.483E+09 |
| 10 μL/g | | 5782 | 7.793E+09 |
| IV, Single dose | | 5788 | 3.307E+09 |
| | | Mean | 6.236E+09 |
| | | SEM | 1.195E+09 |
| Group 10 | Y | 5702 | 3.083E+09 |
| MC3-Form-6 | | 5726 | 6.548E+09 |
| 1.4 mg/kg | | 5769 | 8.508E+09 |
| 10 μL/g | | 5774 | 7.457E+09 |
| IV, Single dose | | 5781 | 5.539E+09 |
| | | Mean | 6.227E+09 |
| | | SEM | 9.267E+08 |
| Group 12 | Y | 5703 | 2.731E+09 |
| MC3-Form-7 | | | |
| 2.8 mg/kg | | 5711 | 4.297E+09 |
| 10 μL/g | | 5730 | 8.090E+09 |
| IV, Single dose | | 5789 | 9.780E+09 |
| | | Mean | 6.225E+09 |
| | | SEM | 1.634E+09 |
| Group 13 | Y | 5719 | 6.283E+09 |
| PBS | | | |
| 10 μL/g | | | |
| IV, Single dose | | | |
| | | Mean | 6.283E+09 |
| | | SEM | — |

Note:
^{a}days after the start of treatment.

This study was designed to assess the cancer-activated gene expression using different delivery formulations, with an LNP shown to be highly effective at delivery in the liver. One cohort (Table 5, Groups 1, 2, 9, and 14) used a secreted embryonic alkaline phosphatase (SEAP) reporter protein to study the activation of the AFP-3 promoter versus the Survivin (BIRC5) promoter. The other groups contained a lead imaging reporter, HSV-sr39tk with a 9-amino acid epitope tag (hemagglutinin) fused to the terminus, a modification that is commonly used to study the expression levels of proteins. The hemagglutinin (HA) tag allows for the use of high affinity anti-HA antibodies to study the protein expression of sr39tk through immunohistochemistry (IHC).

TABLE 5
Experimental Groups in Hep3B Orthotopic Liver Tumor Study

| | | | | | | | Dosing | |
|---|---|---|---|---|---|---|---|---|
| | | | | | Dose | Dosing | Volume | |
| Group | N | Tumor | Treatment | Delivery | (mg/kg) | Route | (mL/kg) | Schedule |
| 1 | 5 | N | NP003 | LNP | 1.4 | IV | 10 | single dose |
| | | | (BIRC5-SEAP) | | | | | |
| 2 | 5 | Y | NP003 | LNP | 1.4 | IV | 10 | single dose |
| | | | (BIRC5-SEAP) | | | | | |
| 3 | 2 | N | NP357 | LNP | 0.7 | IV | 5 | single dose |
| | | | (AFP-3-sr39tk) | | | | | |
| 4 | 5 | Y | NP357 | LNP | 0.7 | IV | 5 | single dose |
| 5 | 2 | N | NP357 | LNP | 2.8 | IV | 10 | single dose |
| 6 | 5 | Y | NP357 | LNP | 2.8 | IV | 10 | single dose |
| 7 | 5 | Y | NP357 | LNP | 1.4 | IV | 10 | single dose |
| 8 | 5 | Y | NP357 | LNP | 0.7 | IV | 10 | single dose |
| 9 | 5 | Y | NP041 | LNP | 1.4 | IV | 10 | single dose |
| | | | (AFP-3-SEAP) | | | | | |
| 10 | 5 | Y | NP355 | LNP | 1.4 | IV | 10 | single dose |
| | | | (CAG-sr39tk) | | | | | |
| 11 | 2 | N | NP357 | LNP | 2.8 | IV | 10 | single dose |
| 12 | 4 | Y | NP357 | LNP | 2.8 | IV | 10 | single dose |
| 13 | 1 | Y | NA | LNP | NA | IV | 10 | single dose |
| 14 | 5 | N | NP041 | LNP | 1.4 | IV | 10 | single dose |
| | | | (AFP-3-SEAP) | | | | | |

### SEAP Results

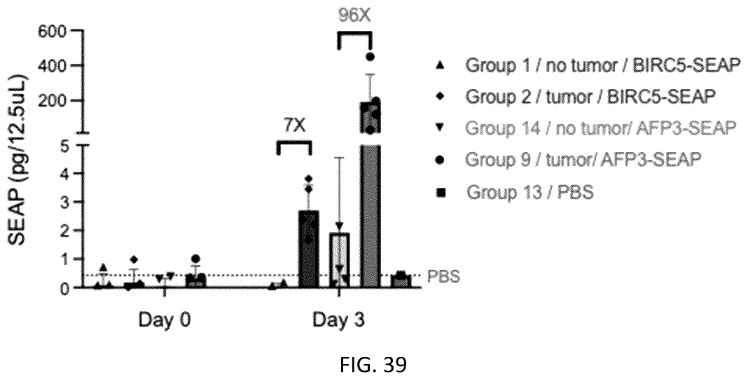

Mice were IV-dosed with EM-40 formulated reporter constructs containing the SEAP reporter, as described in the previous section. Two different DNA nanoplasmids were used; one was comprised with the Survivin (BIRC5) cancer-activated promoter driving SEAP expression and one with the AFP-3 promoter to drive liver cancer activated expression. Once expressed in cancer cells, SEAP is secreted into the blood and a simple blood draw can be collected to reveal the presence of cancer. As expected, SEAP is secreted into the serum by the construct. Control blood draws from all animals before dosing (Day 0 in FIG. 39) showed undetectable background/basal activity in serum from tumor-bearing and normal mice (below the assay's LLOQ of 0.4 pg/12.5 μL serum). At the day 3 bleed, there was a significant difference in the SEAP biomarker availability in serum between non-tumor and tumor mice dosed with the same formulation. For mice dosed with Survivin, the non-tumor animals still showed undetectable background levels of SEAP, and a 7-fold increase over background expression in tumor-bearing mice. While there was a small amount of the reporter SEAP in the non-tumor mice dosed with AFP-3-SEAP, the fold-activation in tumor-bearing mice was higher, at nearly 100-fold the average SEAP expression in the non-tumor background.

### IHC Results

Additional experiments were performed to determine which cells from a target organ contributed to the strong SEAP signal driven from the modified AFP3 promoter in the DNA nanoplasmids. The sequences encoding for SEAP were removed from the DNA nanoplasmid and replaced with sequences encoding for a version of the sr39TK PET Reporter Gene that had been modified with a HA (hemagglutinin) tag—a 9 bp epitope tag. Using antibodies against HA, IHC was performed on formalin fixed paraffin embedded (FFPE) liver tissues using a commonly available anti-HA antibody.

Mice were implanted with liver orthotopic tumors of Hep3B as previously described. EM-040 formulated DNA nanoplasmids that are comprised of the modified AFP-3 promoter to drive the expression of the HA-tagged sr39Tk PET Reporter Gene were injected systemically into the mice. Following 3 days of expression, the mice were sacrificed, their livers were harvested and then processed for IHC staining using the anti-HA antibody. H&E staining which can help distinguish different tissue structures and cell types within a sample, and correlate with expression by IHC to structural location and cell type was also performed. Control-stained sections of tumors and normal left & right lobes of the liver from mice dosed with a non-HA tag expressing construct (in this case BIRC5-SEAP) showed no non-specific staining, demonstrating that the method used specifically and accurately detected only the sr39tk-HA reporter from the construct.

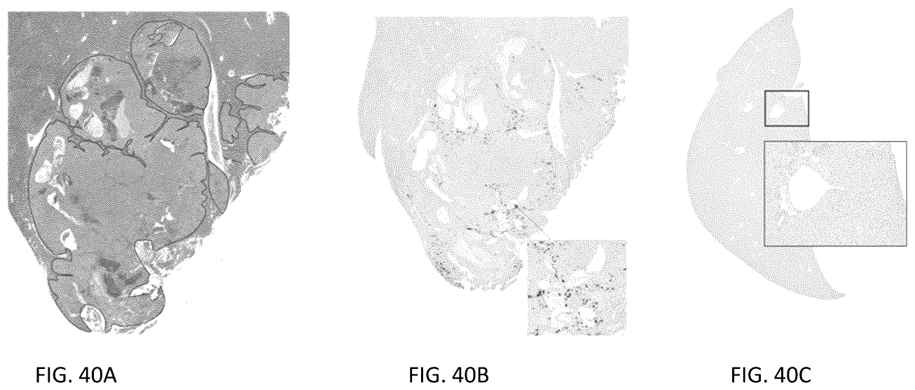

Tumor sections from AFP-3-sr39tk dosed mice (FIGS. 40A-40C) showed strong expression of the construct in a significant portion of cells within the tumor, at both the 2.8 and 1.4 mg/kg dose levels, with no detected expression in left lobe cells bordering the tumor, or the non-tumor right lobe of the liver within the same mice.

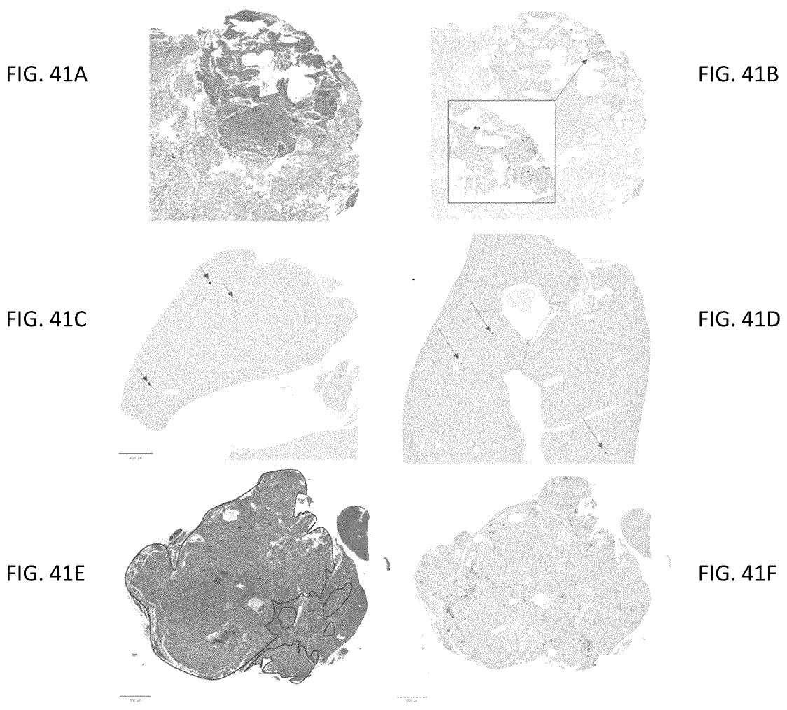

The mice dosed with CAG-sr39tk was similarly studied. Because CAG is a very strong and constitutive promoter, it should accurately exhibit where delivery and expression is possible. While IHC is not quantitative by nature, the qualitative assessment of the tumors (as shown in FIGS. 41A-41F) showed that the CAG-driven construct exhibited equivalent levels of expression in tumors to the AFP-3 promoter, which was remarkable given that that CAG is considered one of the strongest constitutive promoters available in gene therapy. CAG expression was also preferentially localized to the tumor tissue as opposed to normal hepatocytes in the left or right lobe of the liver (possibly indicating that the nature of the highly vascularized tissue helps distribute the vector preferentially to the tumor tissues versus normal), but did show strong expression in disperse single cells in representative left and right lobe sections which were not observed with the more specific AFP-3 (FIGS. 41C and 41D).

### Conclusion

These series of experiments demonstrate the utility of the cancer-specific gene expression in an orthotopic liver tumor model, demonstrating delivery to primary liver tumors as well as activation in the context of a human liver cancer cell. The LNP formulation demonstrates highly effective delivery to tumor cells upon IV dosing.

The AFP-3 promoter showed a nearly 100-fold higher activation in the blood marker SEAP than the BIRC5 promoter in the Hep3B-model, and IHC analysis also showed highly specific and strong expression in tumor cells and not in normal liver cells. The highly qualitative IHC data demonstrated strong levels of activation of the AFP-3 promoter and the ability of the combined components to deliver and express in a cancer-specific manner.

### Example 4: Benign Versus Malignant, Inflammation and Specificity

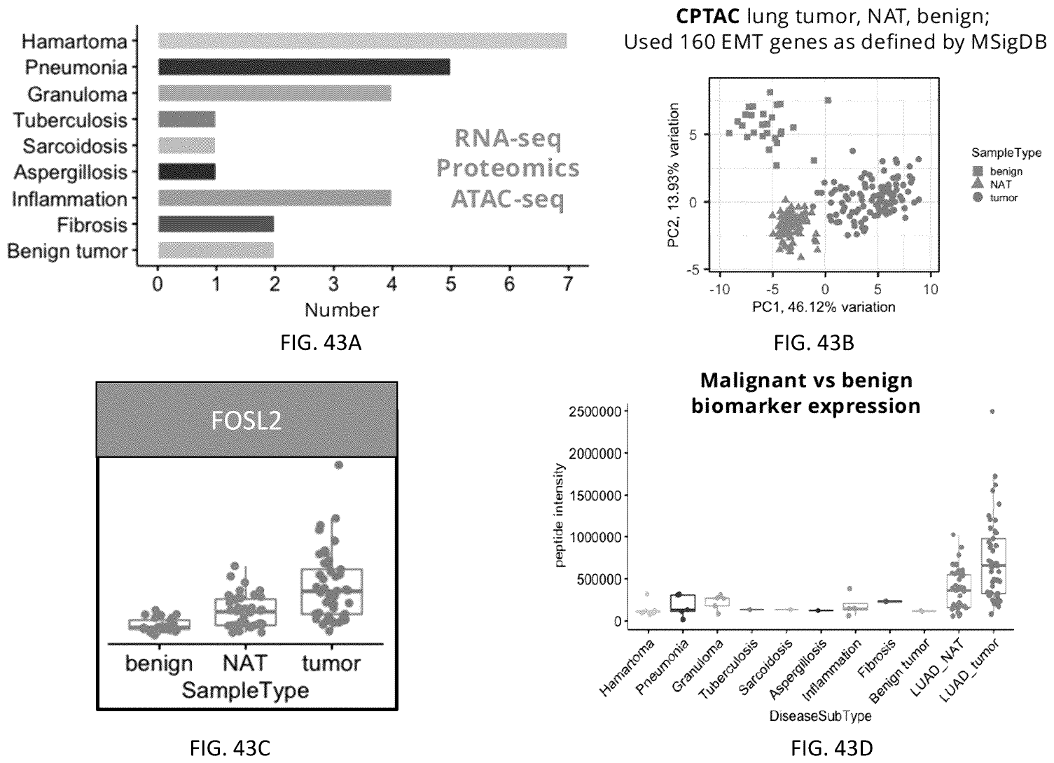

Multi-omics (RNA-seq, proteomics, and ATAC-seq) methodology was used to analyze benign tissue/cell samples. FIG. 43A shows number of different benign tissue/cell samples used for multi-omics analysis. Details of multi-omics methodology was described in Examples 1 and 2. Analysis of 160 Epithelial-Mesenchymal Transition (EMT) genes defined by the Molecular Signatures Database (MsigDB; see Liberzon A., et al. The Molecular Signatures Database hallmark gene set collection. Cell Syst. 2015 Dec. 23;1 (6): 417-425) using multi-omics and principal component analysis (PCA) demonstrated a transcriptomic difference between malignant human lung cancer (Clinical Proteomic Tumor Analysis Consortium (CPTAC) lung tumor) and benign lesions (NAT), and internal benign) (FIGS. 43B-43D).

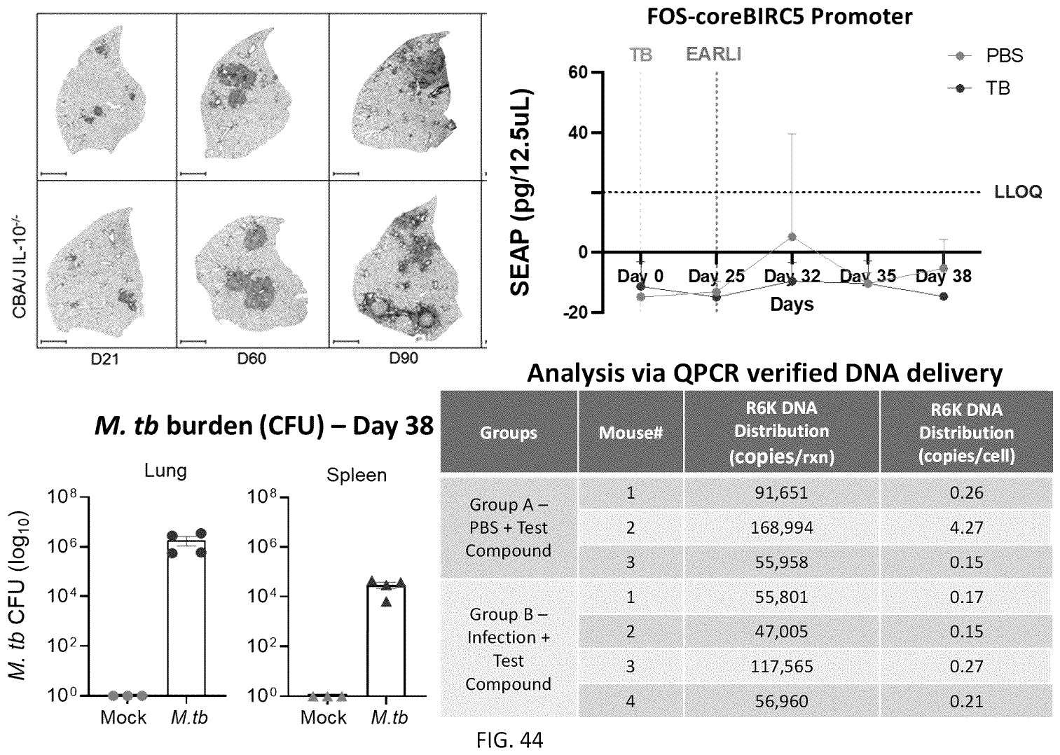

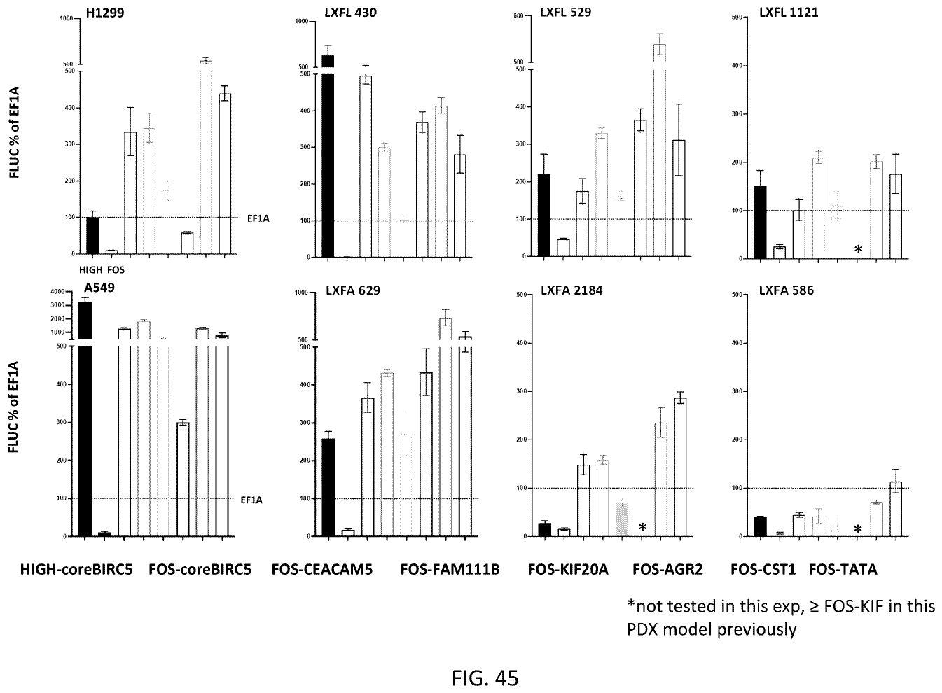

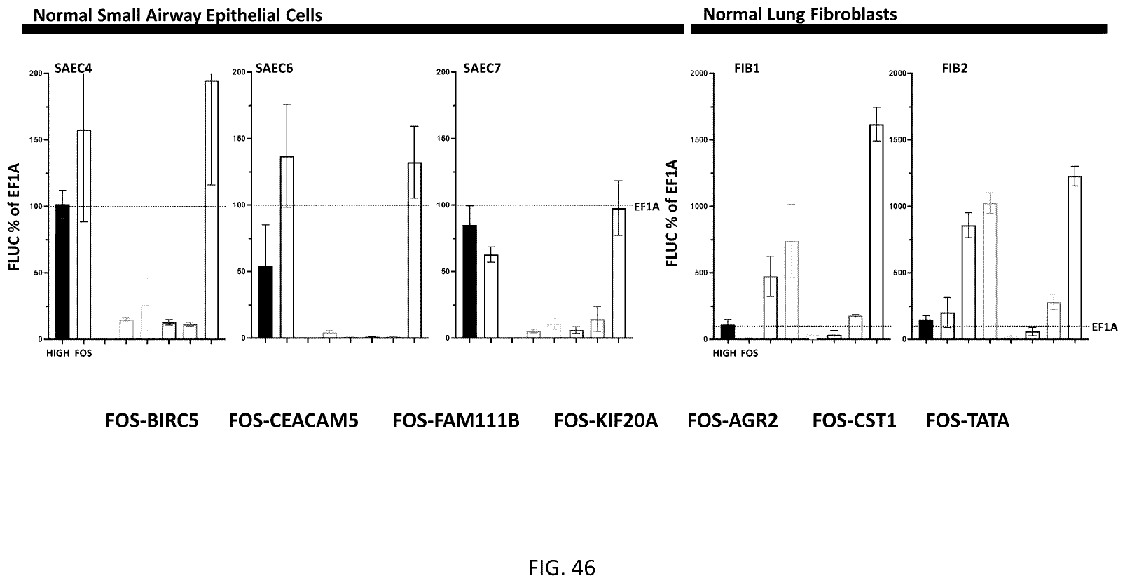

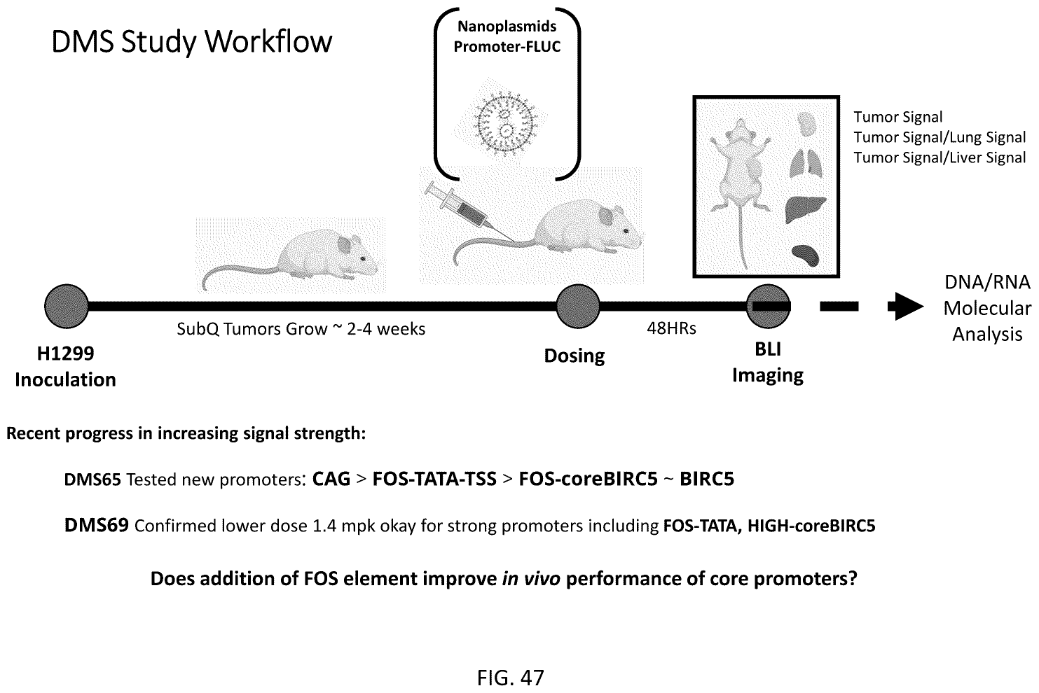

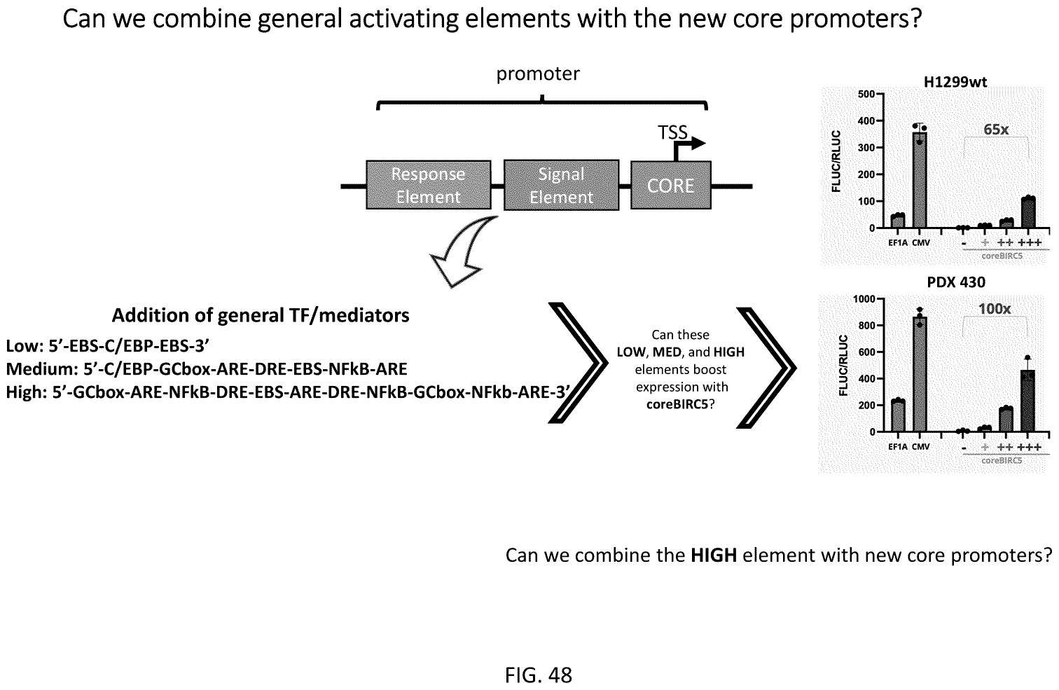

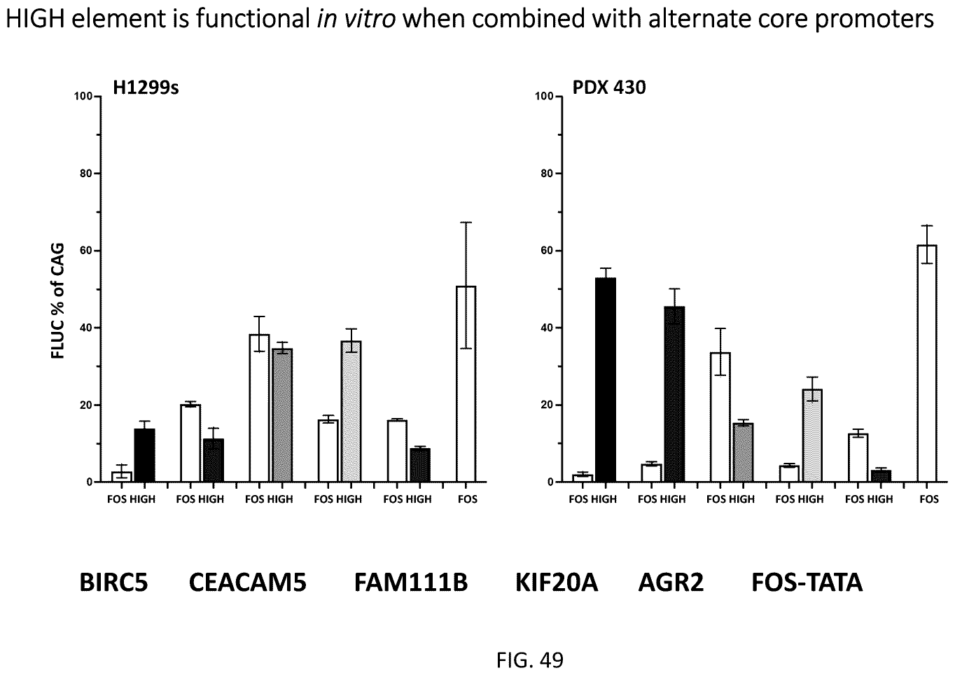

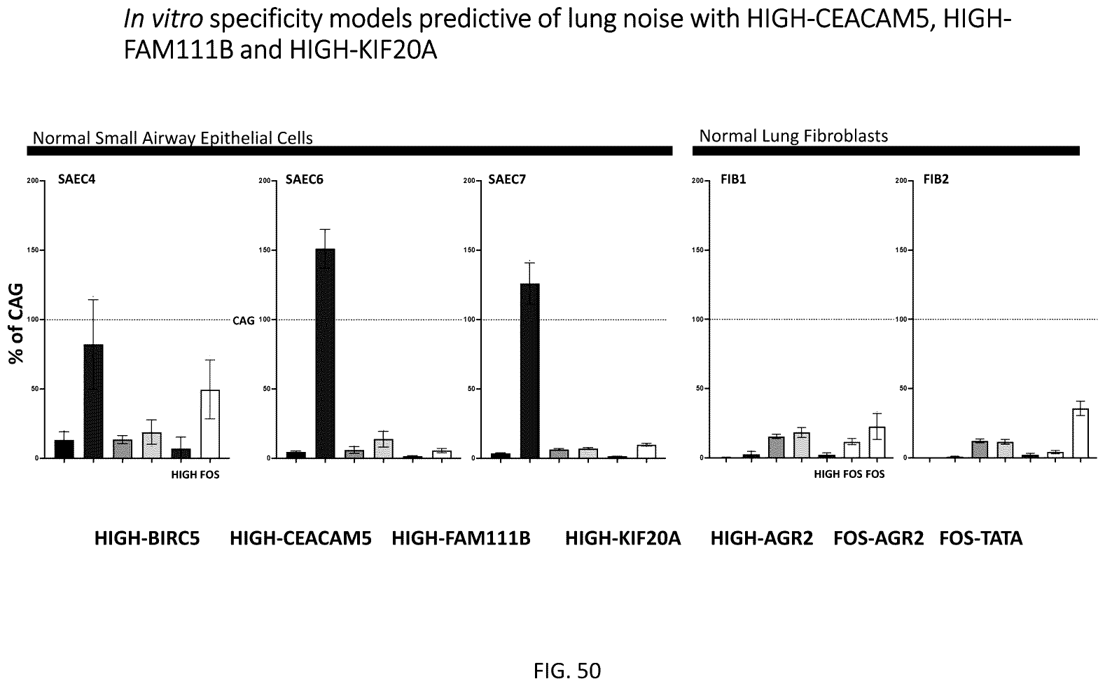

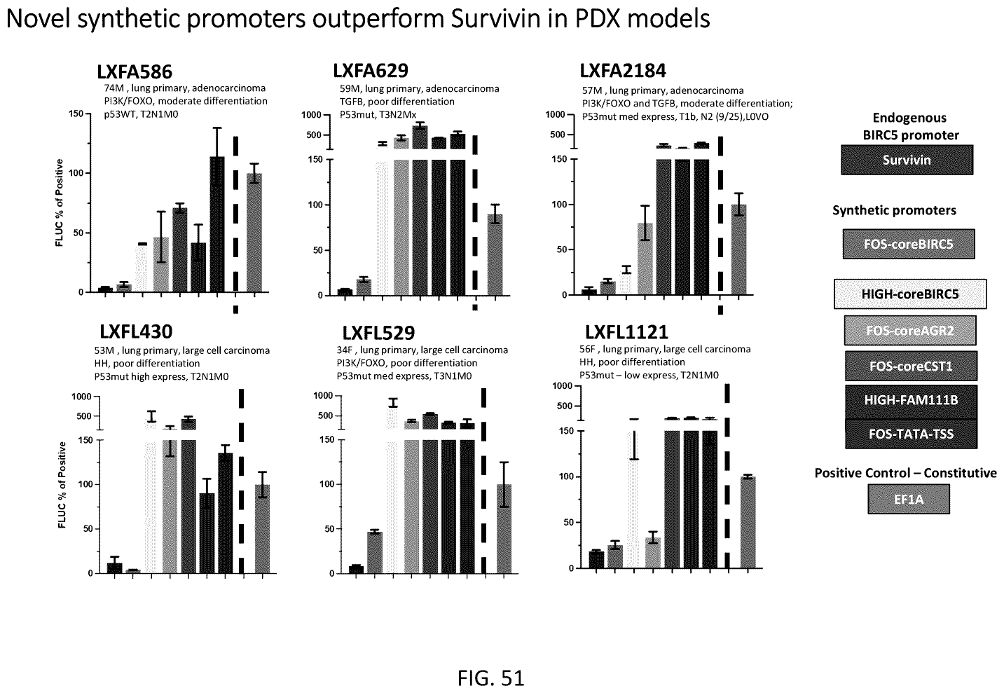

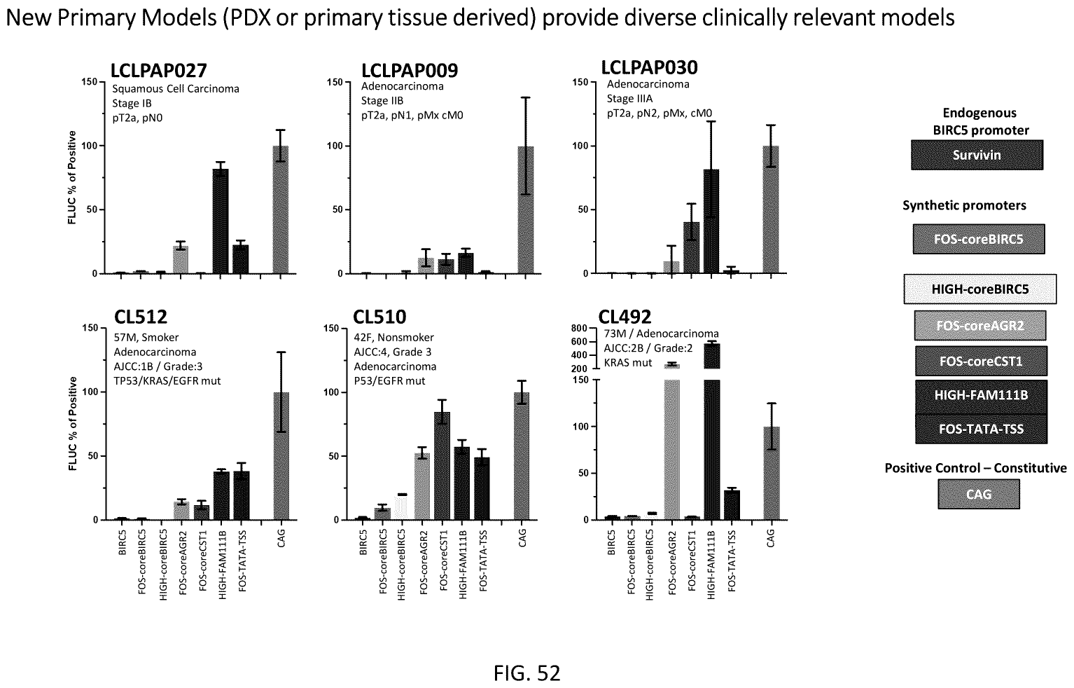

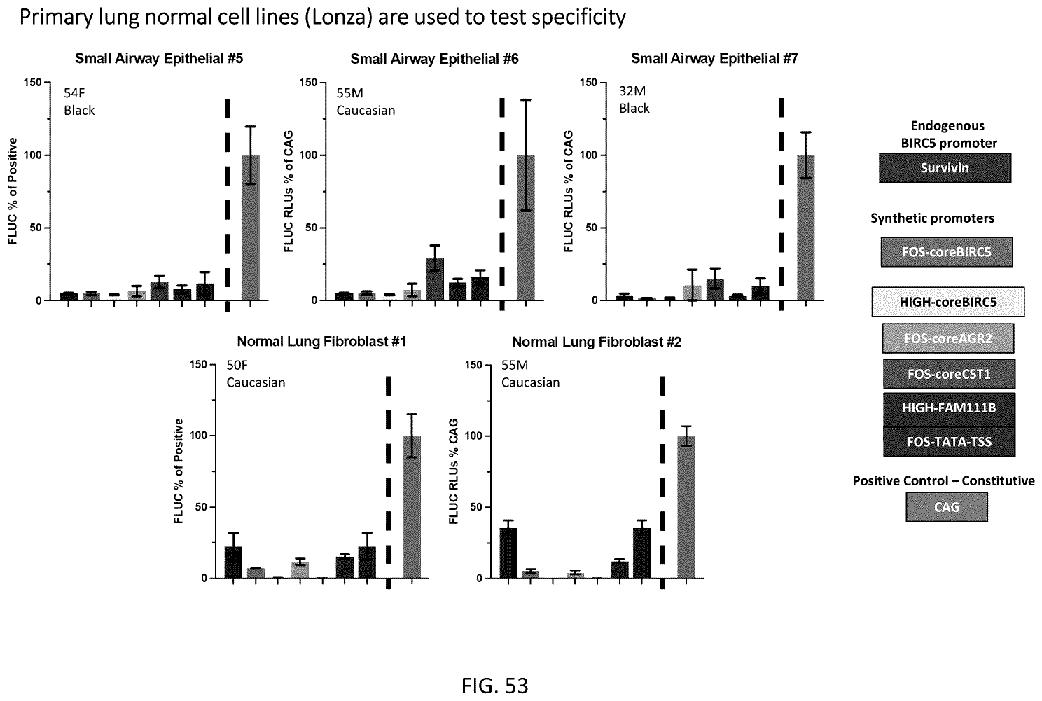

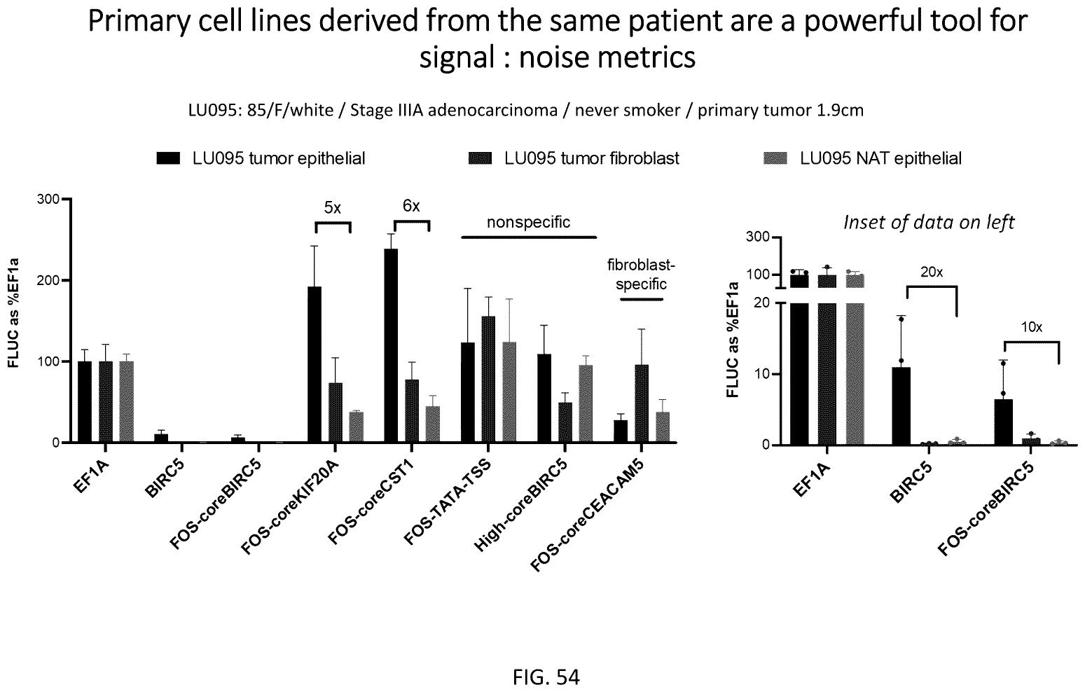

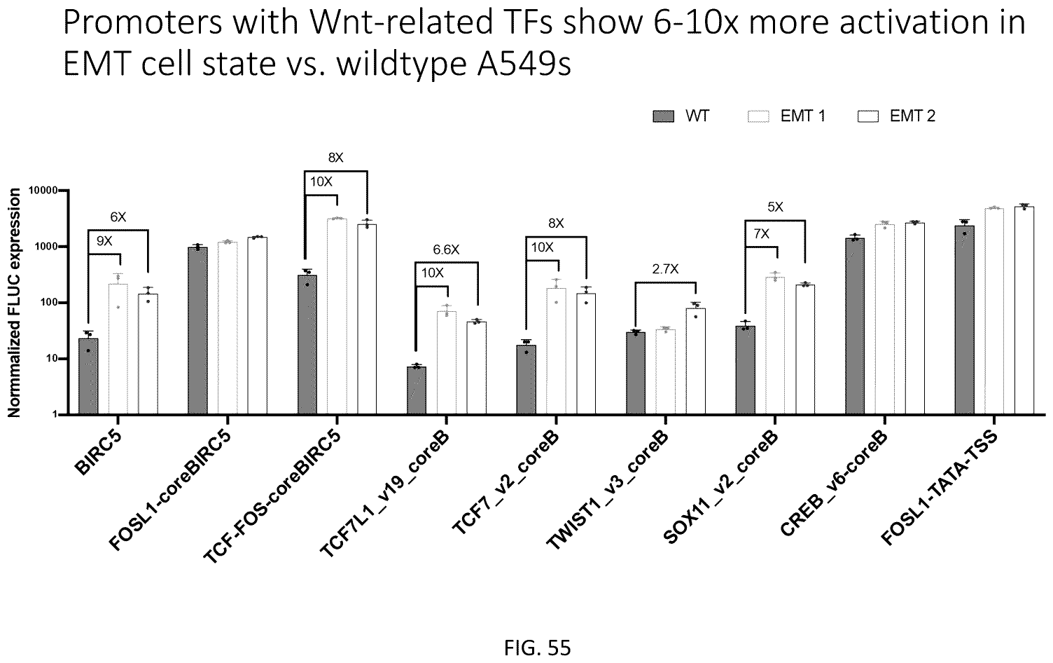

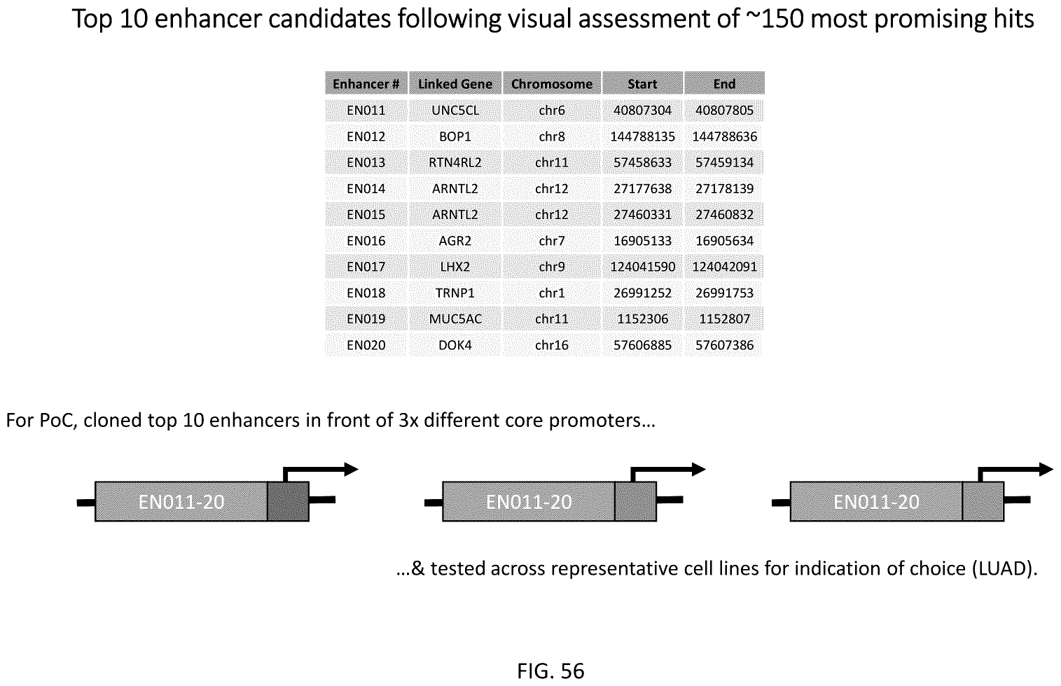

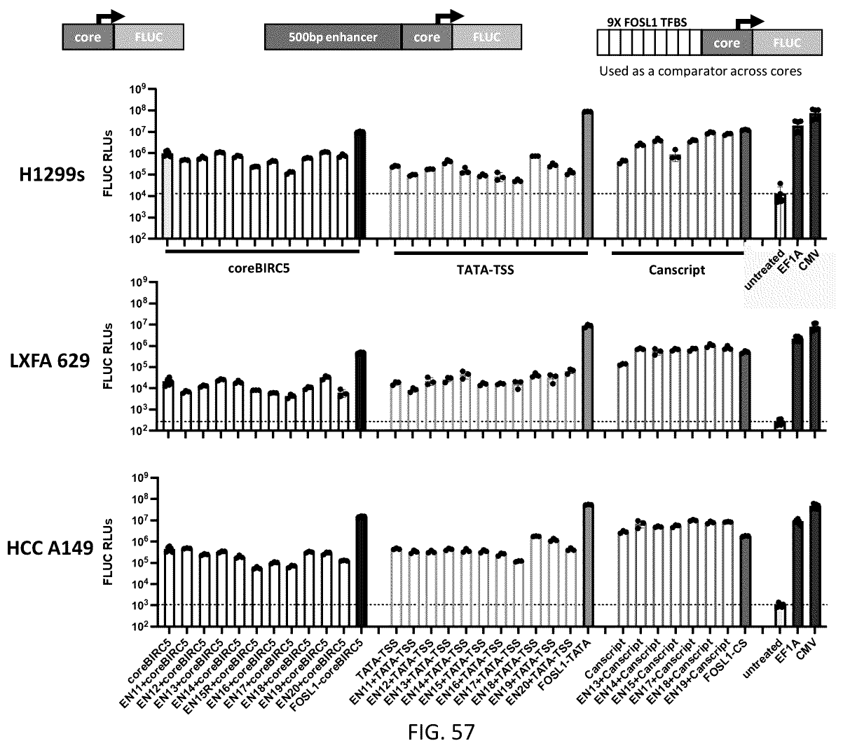

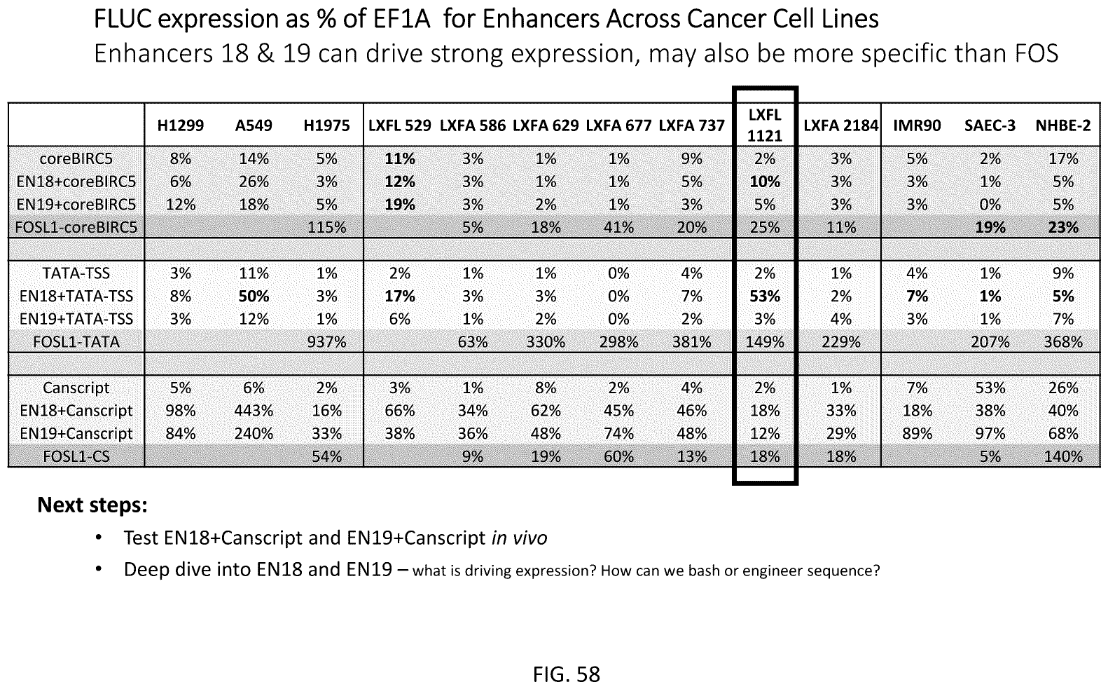

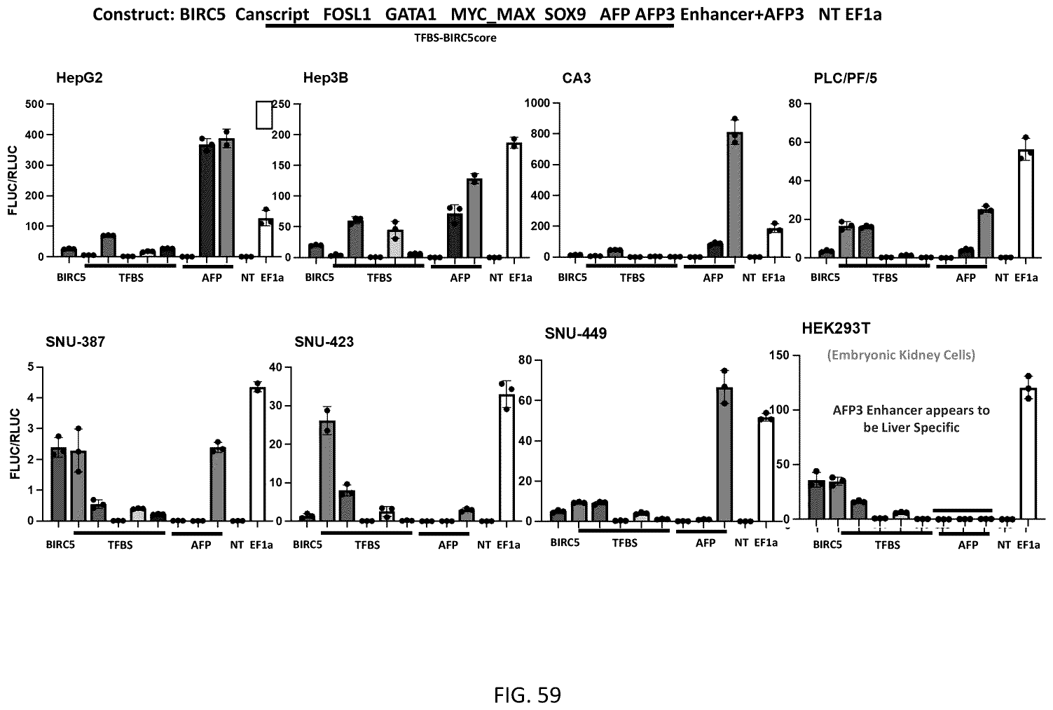

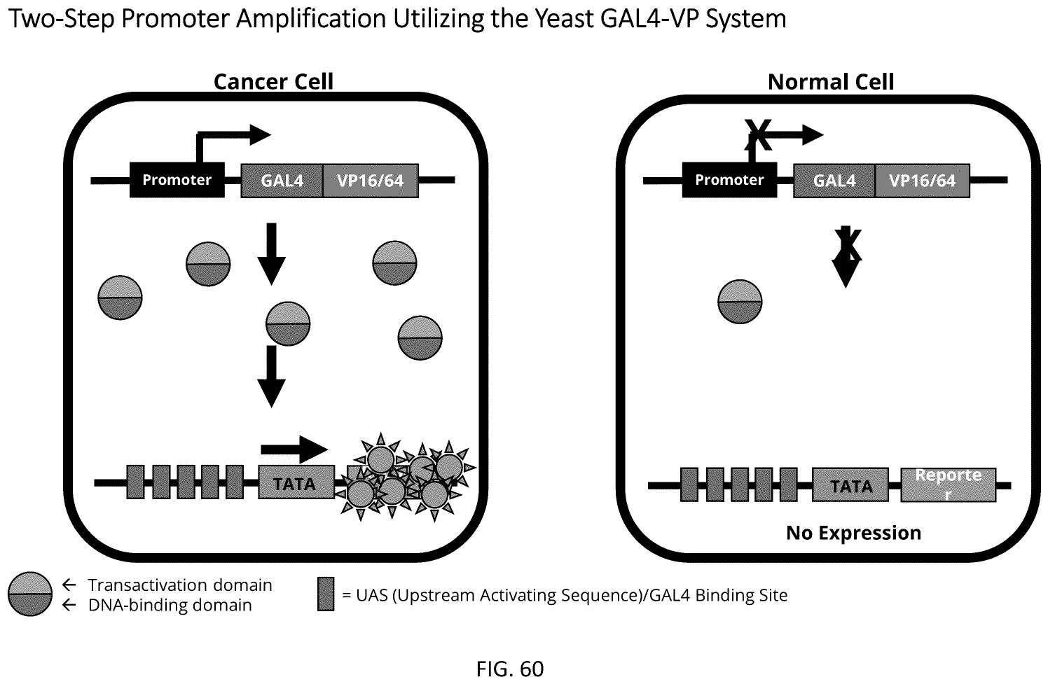

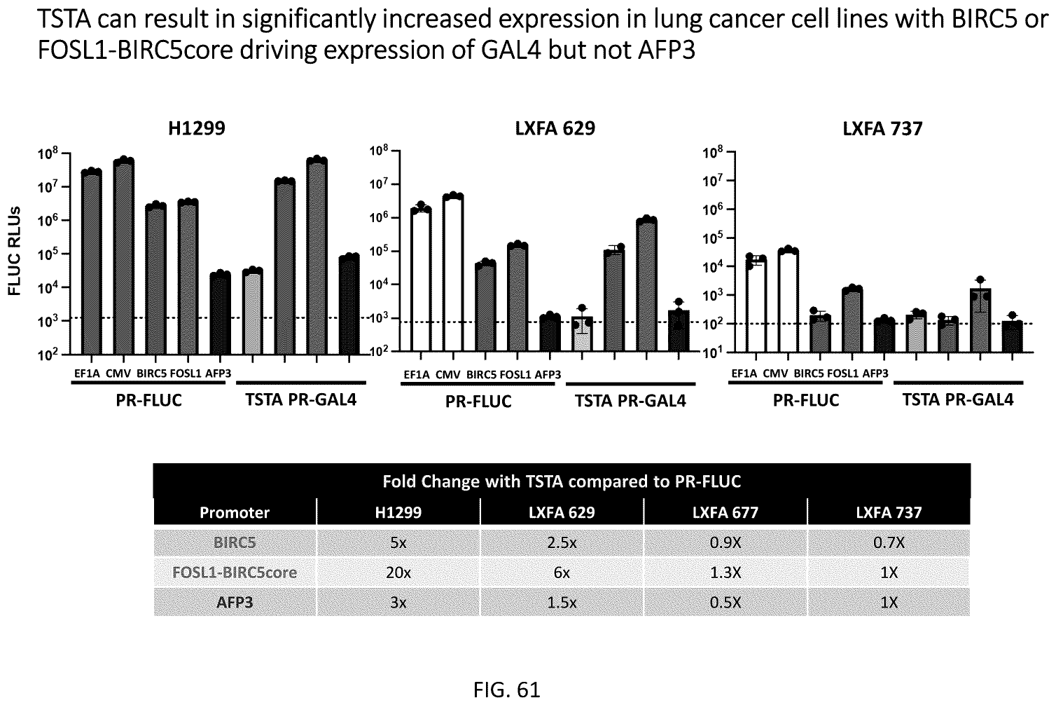

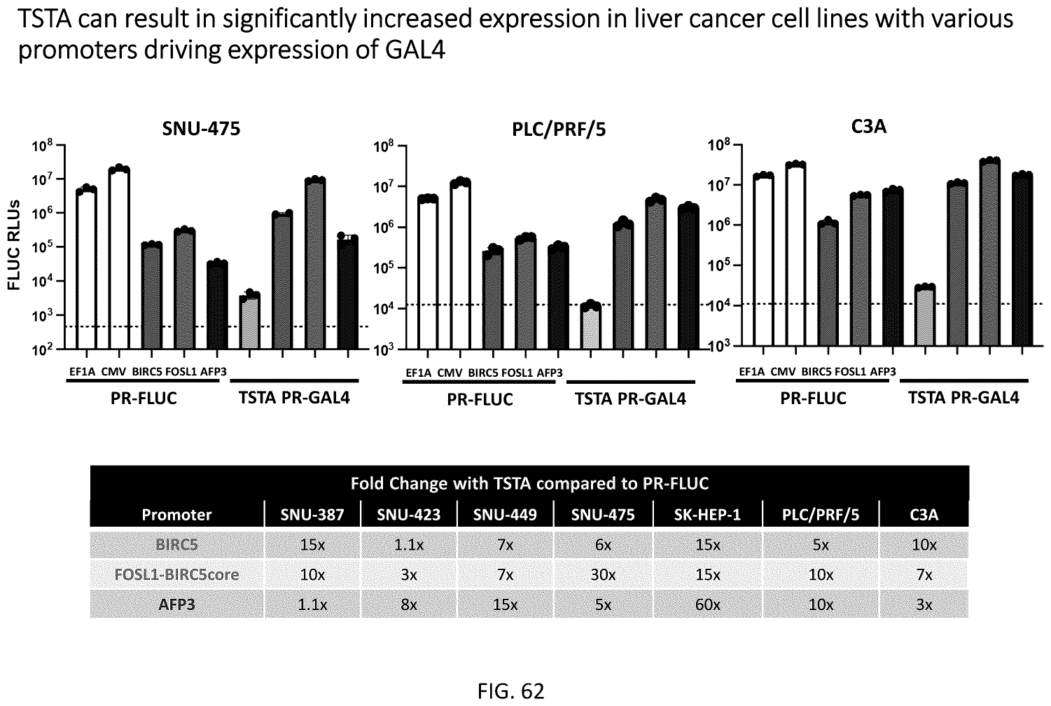

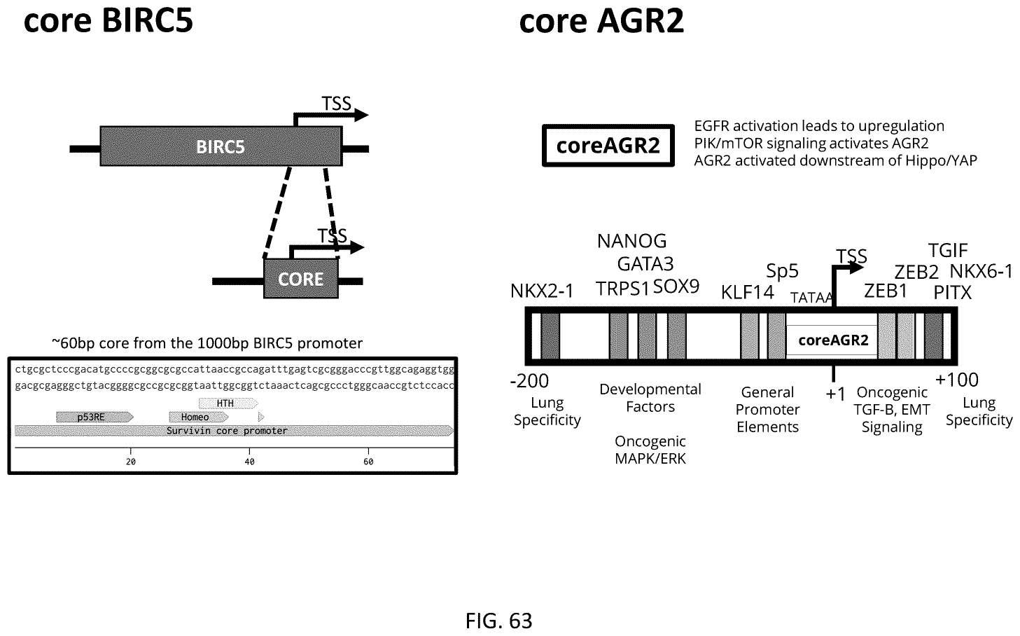

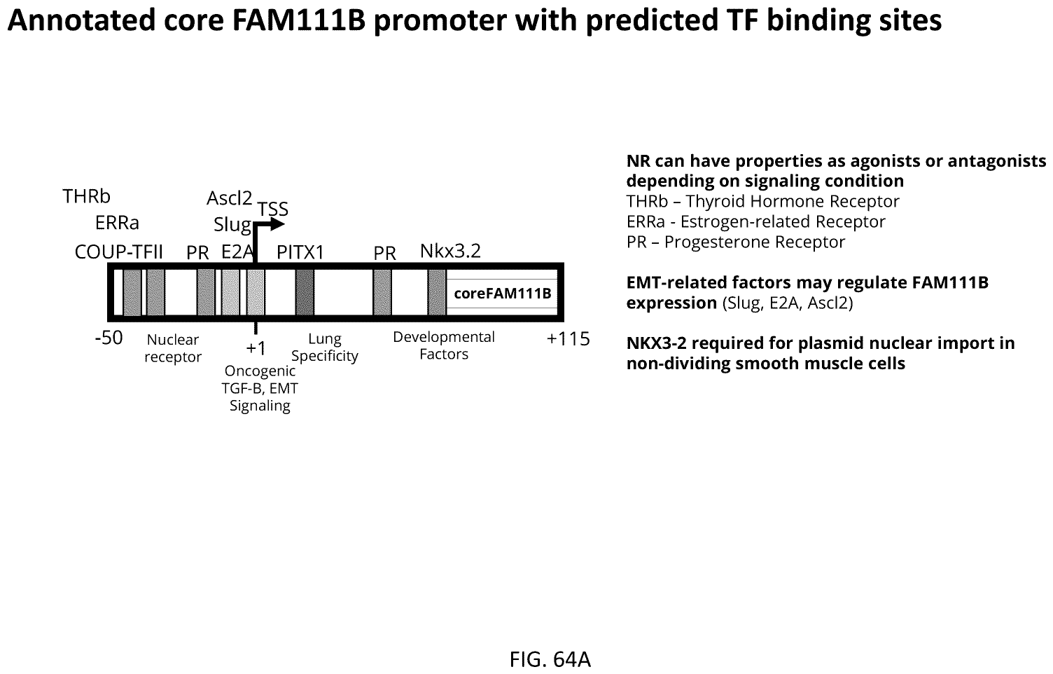

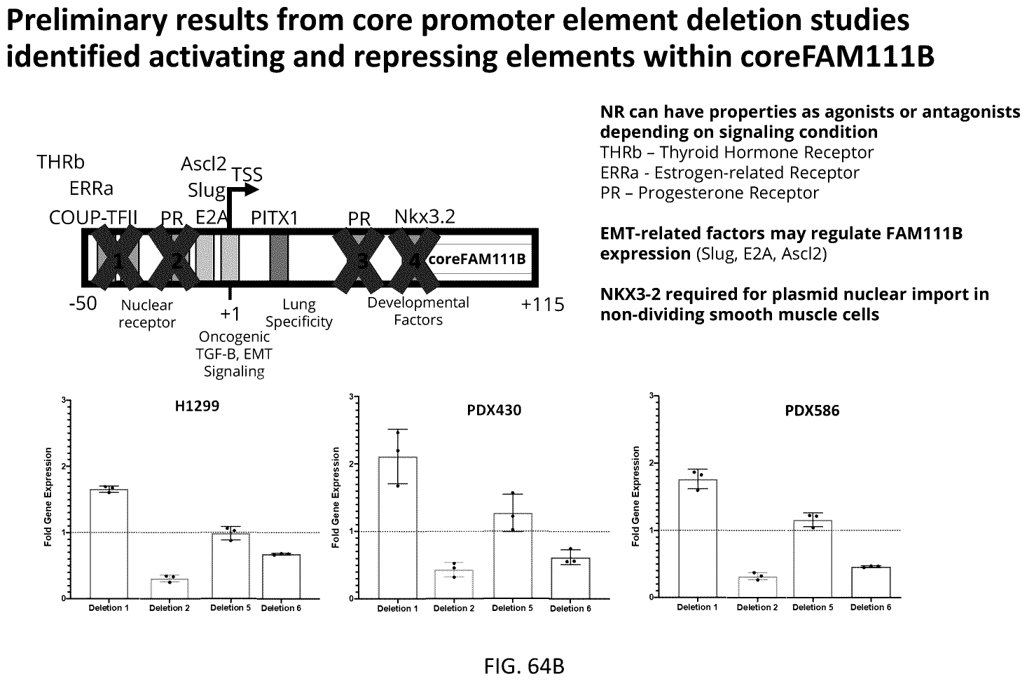

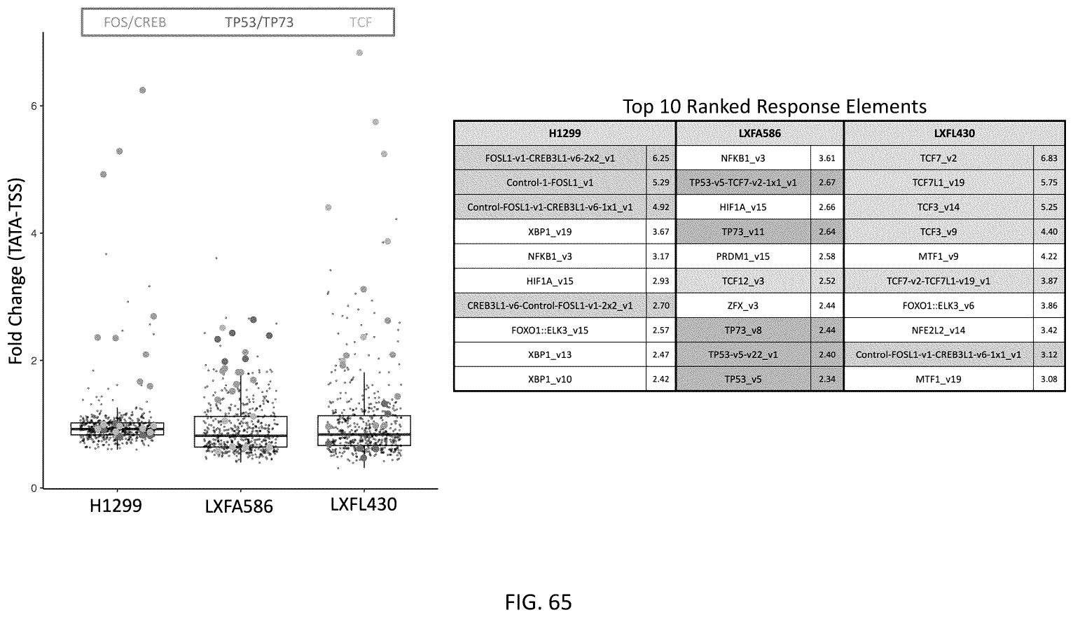

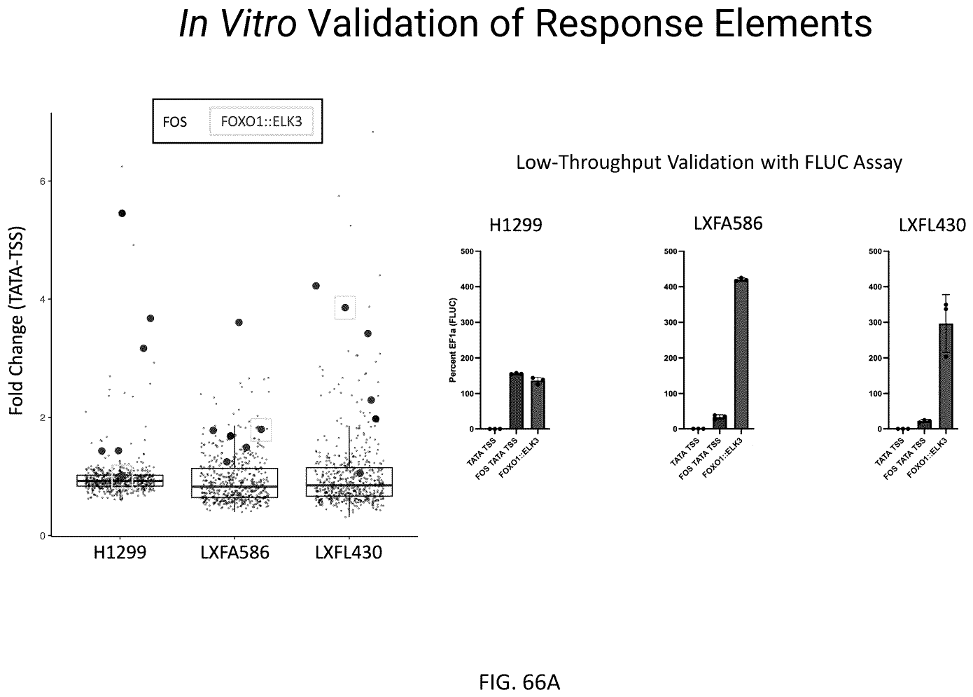

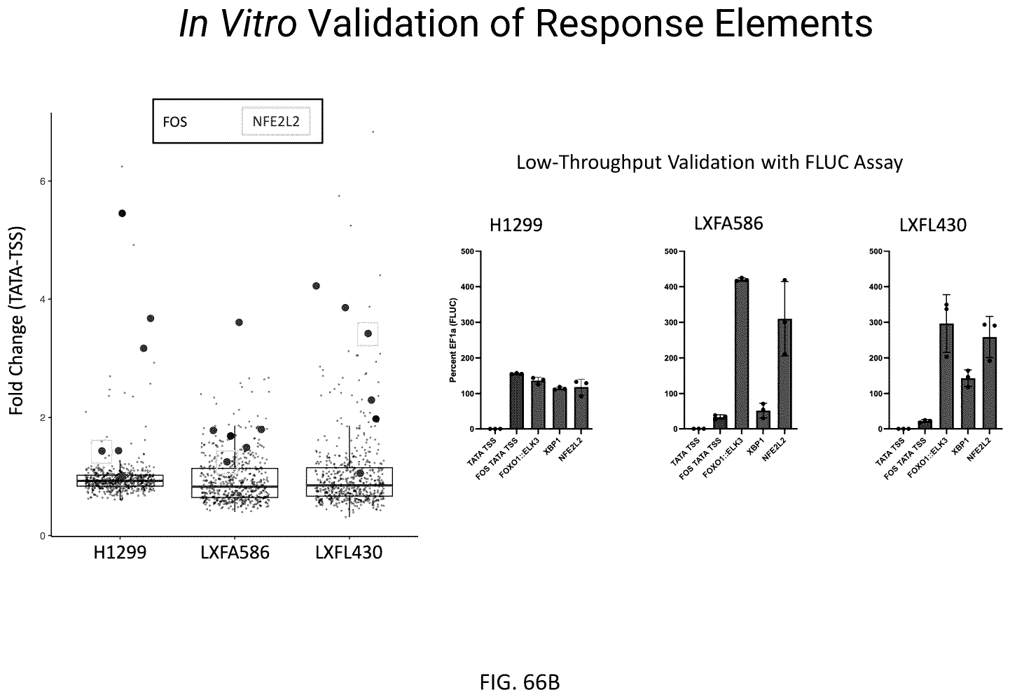

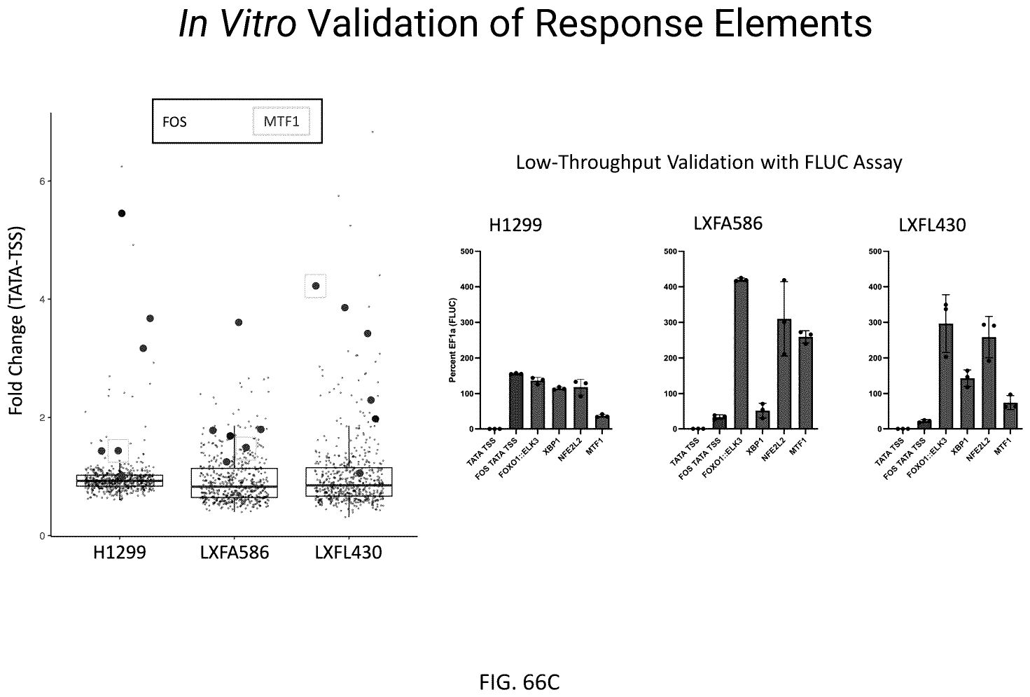

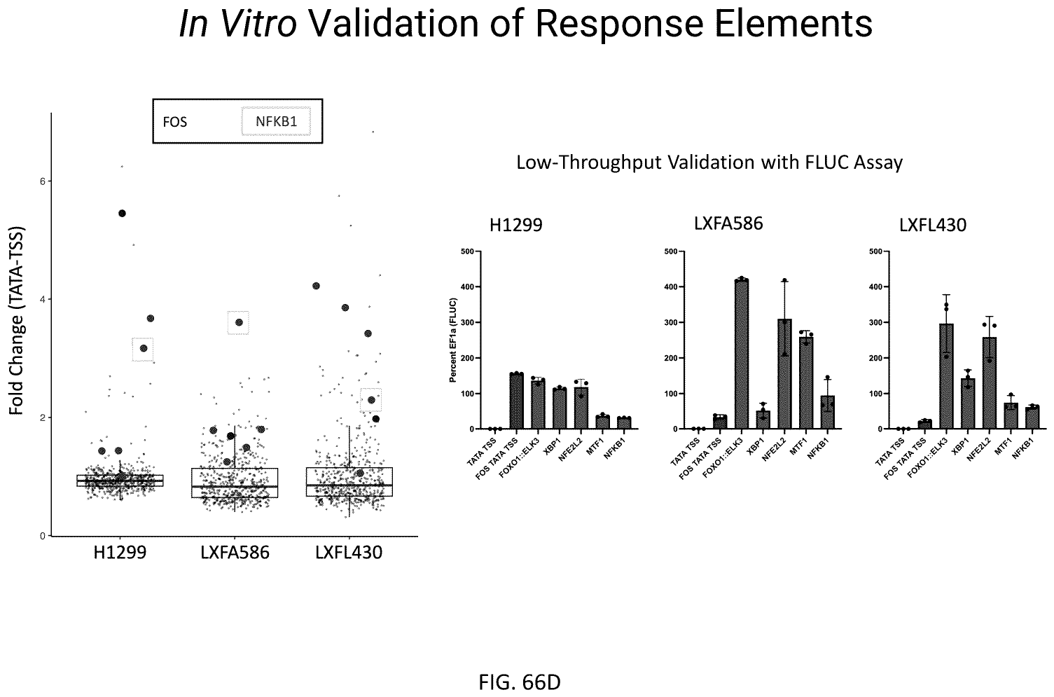

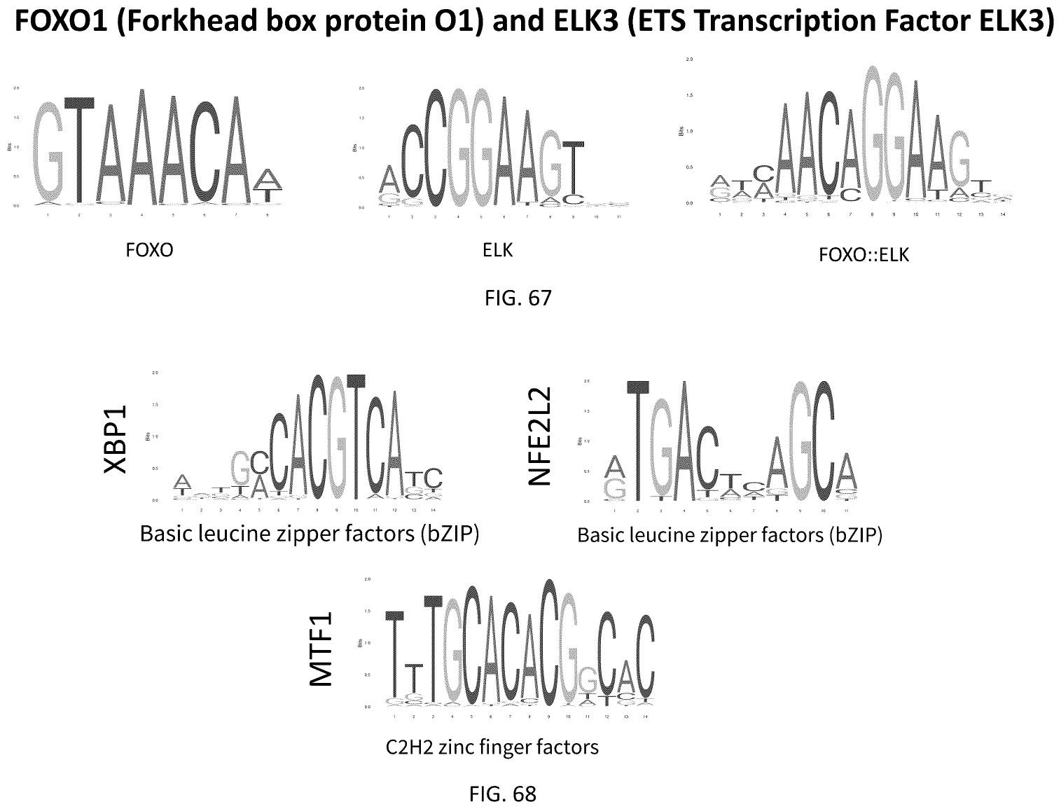

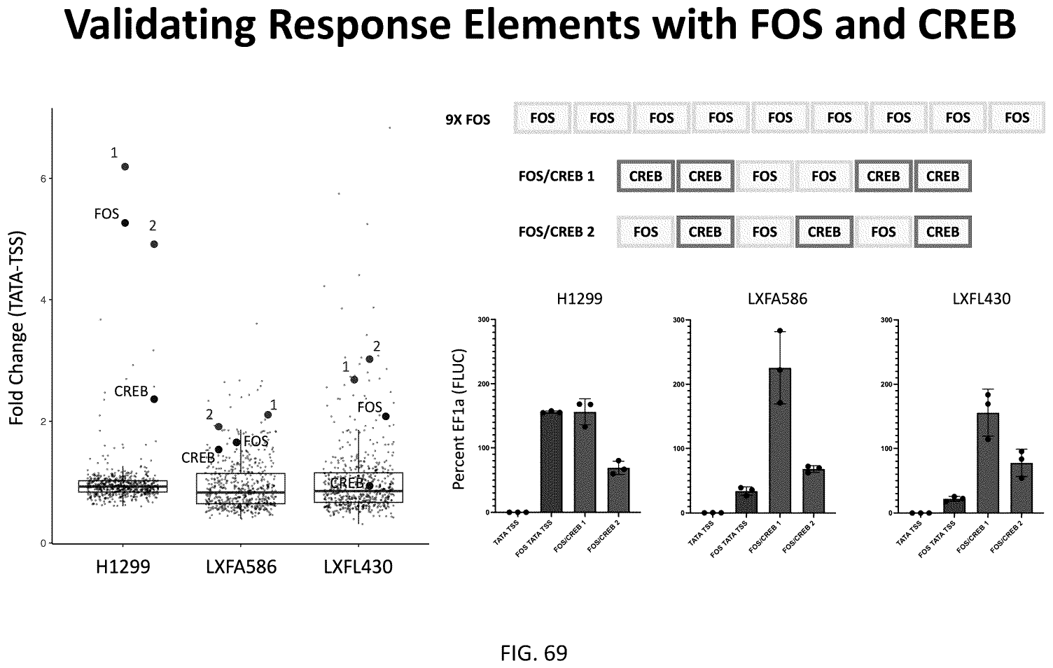

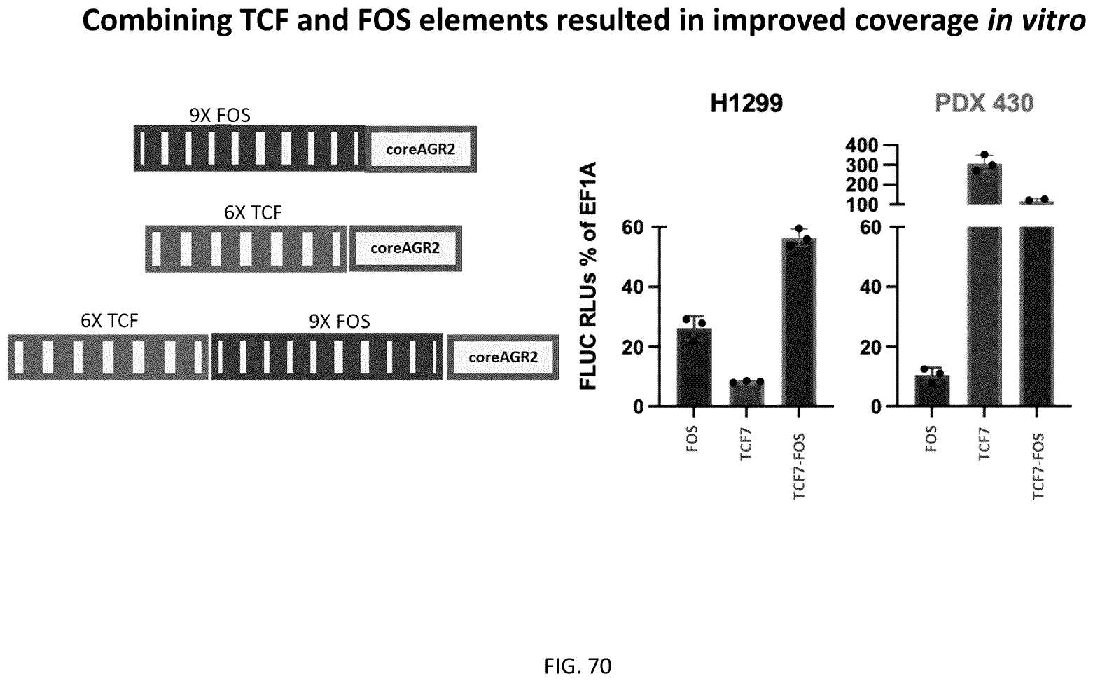

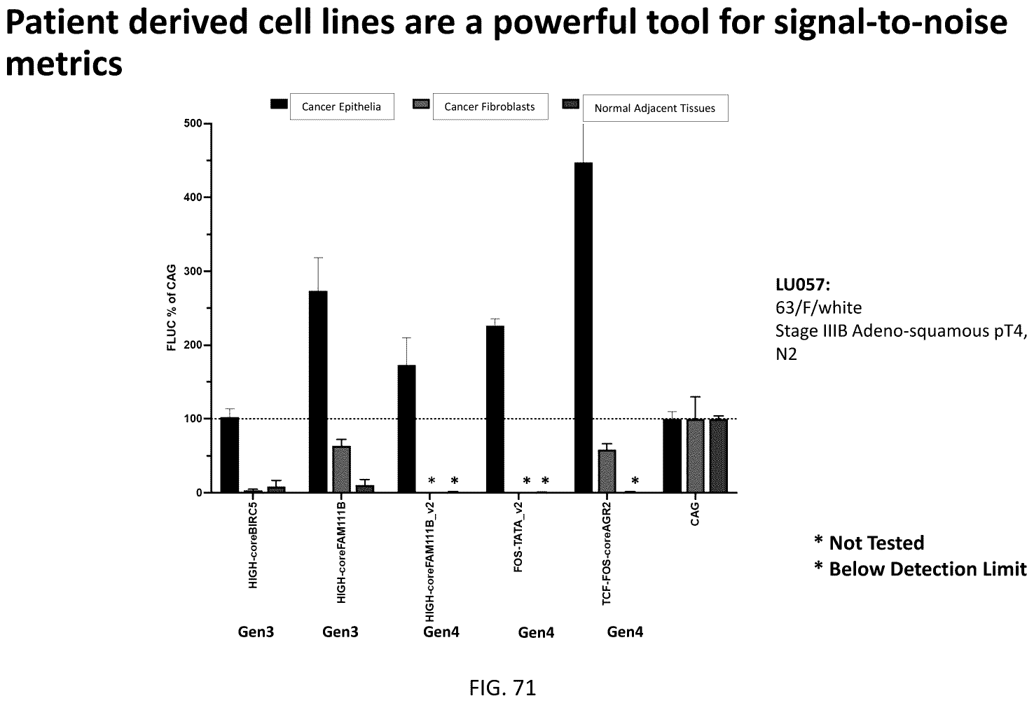

Next, using CBA/J mice model infected with Mycobacterium tuberculosis (M. tb; S. Major, J. Turner, and G. Beamer. Tuberculosis in CBA/J Mice. Veterinary Pathology 2013 50:6, 1016-1021), reporter gene expression driven by FOS-core-BIRC5 synthetic promoter was analyzed. There was no expression of reporter gene in granulomatous lesions caused by M. tb infection in CBA/J mice despite high disease burden (FIG. 44), suggesting there is no cancer-activated expression in granulomas, which is a model of benign tissue lesions.

The examples and embodiments described herein are for illustrative purposes only and various modifications or changes suggested to persons skilled in the art are to be included within the spirit and purview of this application and scope of the appended claims.

### EMBODIMENTS

The following embodiments are not intended to be limiting in any way.

Embodiment 1: A recombinant polynucleotide comprising:
(a) a core promoter comprising a transcription start site (TSS), wherein the core promoter is derived from one or more cancer-responsive genes that are either expressed at a higher level or are more active in cancer cells compared to non-cancer cells and operably linked to an open reading frame (ORF) and
(b) a plurality of binding sites for one or more transcription factors (TFs), wherein said one or more TFs are expressed at higher levels or more active in cancer cells compared to non-cancer cells.

Embodiment 2: A recombinant polynucleotide comprising:
(a) a core promoter comprising a transcription start site (TSS) and two or more promoter elements derived from two or more cancer-responsive genes that are either expressed at a higher level or are more active in cancer cells compared to non-cancer cells and operably linked to an open reading frame (ORF) and
(b) a plurality of binding sites for one or more transcription factors (TFs), wherein said one or more TFs are expressed at higher levels or more active in cancer cells compared to non-cancer cells.

Embodiment 3: The recombinant polynucleotide of Embodiment 1 or 2, further comprising a plurality of enhancers.

Embodiment 4: A recombinant polynucleotide comprising:
(a) a core promoter comprising a transcription start site (TSS), wherein the core promoter is derived from one or more cancer-responsive genes that are either expressed at a higher level or are more active in cancer cells compared to non-cancer cells and operably linked to an open reading frame (ORF) and
(b) a plurality of enhancers.

Embodiment 5: A recombinant polynucleotide comprising:
(a) a core promoter comprising a transcription start site (TSS), wherein the core promoter is derived from one or more cancer-responsive genes that are either expressed at a higher level or are more active in cancer cells compared to non-cancer cells and operably linked to an open reading frame (ORF),
(b) a plurality of binding sites for one or more transcription factors (TFs), wherein said one or more TFs are expressed at higher levels or more active in cancer cells compared to non-cancer cells, and
(c) a plurality of enhancers.

Embodiment 6: The recombinant polynucleotide of any one of embodiments 3-5, wherein said plurality of enhancers are derived from one or more cancer-responsive genes that are either expressed at a higher level or are more active in cancer cells compared to non-cancer cells.

Embodiment 7: The recombinant polynucleotide of any one of embodiments 3-6, wherein the plurality of enhancers are derived from two or more cancer-responsive genes that are either expressed at a higher level or are more active in cancer cells compared to non-cancer cells, wherein one of said plurality of enhancers comprises:
(i) a transcription regulatory element with at least 90% sequence homology to an enhancer consensus sequence of two or more homologous cancer-responsive genes, and/or
(ii) a sequence capable of binding a transcription associated protein as determined by chromatin immunoprecipitation (ChIP) or an in vitro transfection reporter assay.

Embodiment 8: The recombinant polynucleotide of any one of embodiments 1-7, wherein said core promoter further comprises two or more promoter elements derived from two or more cancer-responsive genes that are either expressed at a higher level or are more active in cancer cells compared to non-cancer cells and operably linked to an open reading frame (ORF).

Embodiment 9: The recombinant polynucleotide of any one of embodiments 1-8, wherein said one or more cancer-responsive genes are derived from a human subject.

Embodiment 10: The recombinant polynucleotide of any one of embodiments 6-9, wherein: (a) said core promoter, and (b) said plurality of binding sites for one or more TFs or said plurality of enhancers derived from one or more cancer-responsive genes are not derived from a same cancer-responsive gene.

Embodiment 11: The recombinant polynucleotide of any one of embodiments 7-10, wherein said enhancer consensus sequence of two or more homologous cancer-responsive genes is a consensus sequence of an enhancer sequence derived from two or more cancer-responsive genes that has at least 90% sequence identity between two or more human cancer-responsive genes.

Embodiment 12: The recombinant polynucleotide of any one of embodiments 3-11, wherein at least one of the plurality of enhancers comprises a CpG island.

Embodiment 13: The recombinant polynucleotide of any one of embodiments 3-11, wherein at least one of the plurality of enhancers does not comprise a CpG island.

Embodiment 14: The recombinant polynucleotide of any one of embodiments 1-13, wherein said higher levels of TF expression in cancer cells compared to non-cancer cells is determined by chromatin immunoprecipitation (ChIP).

Embodiment 15: The recombinant polynucleotide of any one of embodiments 1-14, further comprising an open reading frame (ORF), wherein said core promoter is operably linked to said ORF.

Embodiment 16: The recombinant polynucleotide of any one of embodiments 1-15, wherein said plurality of binding sites for one or more TFs are 5′ to said core promoter.

Embodiment 17: The recombinant polynucleotide of any one of embodiments 3-16, wherein said plurality of enhancers are 5′ to said core promoter and 3′ to said plurality of binding sites for one or more TFs, if present.

Embodiment 18: The recombinant polynucleotide of any one of embodiments 1-17, wherein said plurality of binding sites for one or more TFs comprises two or more binding sites for one TF, wherein each of the plurality of binding sites for one or more TFs is sequentially arranged at 5′ to said core promoter in the recombinant polynucleotide.

Embodiment 19: The recombinant polynucleotide of any one of embodiments 1-17, wherein said plurality of binding sites for one or more TFs comprises two or more binding sites for two or more TFs, wherein each of the plurality of binding sites for one or more TFs is non-sequentially arranged at 5′ to said core promoter in the recombinant polynucleotide.

Embodiment 20: The recombinant polynucleotide of any one of embodiments 1-19, wherein said plurality of binding sites for one or more TFs comprise a plurality of TRPS1, MNX1, TWIST1, ETV4, FOSL2, NFIC, EN2, TFDP1, PITX2, TCF7L1, VENTX, HOXB9, DLX1, MYCN, SIX4, TP63, SOX11, E2F8, TFDP1, SURV, TOXE1, EN1, ZBTB7B, SP3, SIX2, XBP1, HIF-1A, CREB3L1, HSF-1, MTF1, NFE2L2, USF2, TP73, USF2, POU2F2, HOXA1, FOXO1, TFAP4, BACH1, E2F4, HOXC10, KLF11, FOXM1, E2F2, RUNX1, SOX4, RREB1, ETV4, HES6, ASCL1, TWIST1, FOXA3, PITX2, HOXB2, EN2, DLX4, GRHL1, FOXA, HIF, E2F6, FOSL1, NF-1, RFX6, EL4, or NFκB TF binding sites.

Embodiment 21: The recombinant polynucleotide of any one of embodiments 1-20, further comprising a spacer element comprising 1-10 nucleotides between each of plurality of binding sites for one or more TFs.

Embodiment 22: The recombinant polynucleotide of any one of embodiments 1-21, wherein said one or more cancer-responsive genes from which said core promoter is derived comprise TCF7, MNX1, HOXC10, TP53, CEACAM5, CEP55, FAM111B, CST1, BIRC5, FOS, TWIST1, E2F2, KIF20A, or ETV4.

Embodiment 23: The recombinant polynucleotide of any one of embodiments 1-22, wherein said one or more cancer-responsive genes from which said core promoter is derived comprise two or more of TCF7, MNX1, HOXC10, TP53, CEACAM5, CEP55, FAM111B, CST1, BIRC5, FOS, TWIST1, E2F2, KIF20A, or ETV4.

Embodiment 24: The recombinant polynucleotide of any one of embodiments 1-22, wherein said one or more cancer-responsive genes from which said core promoter is derived comprise TCF7 and HOXC10.

Embodiment 25: The recombinant polynucleotide of any one of embodiments 1-22, wherein said one or more cancer-responsive genes from which said core promoter is derived comprise TP53 and CEP55.

Embodiment 26: The recombinant polynucleotide of any one of embodiments 1-22, wherein said one or more cancer-responsive genes from which said core promoter is derived comprise FAM111B and KIF20A.

Embodiment 27: The recombinant polynucleotide of any one of embodiments 1-22, wherein said one or more cancer-responsive genes from which said core promoter is derived comprise BIRC5 and E2F2.

Embodiment 28: The recombinant polynucleotide of any one of embodiments 1-22, wherein said one or more cancer-responsive genes from which said core promoter is derived comprise CEACAM5 and TWIST1.

Embodiment 29: The recombinant polynucleotide of any one of embodiments 1-28, wherein said core promoter comprises a region from about −300 bp to +100 bp relative to said TSS.

Embodiment 30: The recombinant polynucleotide of any one of embodiments 3-29, wherein said plurality of enhancers comprises at least two enhancer sequences, wherein each of said at least two enhancer sequences comprises (i) the same enhancer sequences, (ii) different enhancer sequences, or (iii) a combination thereof.

Embodiment 31: The recombinant polynucleotide of embodiment 30, wherein each of said at least two enhancer sequences is sequentially arranged at 5′ to said core promoter in the recombinant polynucleotide.

Embodiment 32: The recombinant polynucleotide of embodiment 30, wherein each of said at least two enhancer sequences is sequentially arranged at 5′ to said core promoter and at 3′ to said plurality of binding sites of one or more TFs, if present, in the recombinant polynucleotide.

Embodiment 33: The recombinant polynucleotide of embodiment 30, wherein each of said at least two enhancer sequences comprises (ii), wherein each of said plurality of enhancers comprising different enhancer sequences is non-sequentially arranged at 5′ to said core promoter in the recombinant polynucleotide.

Embodiment 34: The recombinant polynucleotide of embodiment 30, wherein each of said at least two enhancer sequences comprises (ii), wherein each of said plurality of enhancers is non-sequentially arranged at 5′ to said core promoter and at 3′ to said plurality of binding sites for one or more TFs, if present, in the recombinant polynucleotide.

Embodiment 35: The recombinant polynucleotide of embodiment 30, wherein each of said at least two enhancer sequences comprises (iii), wherein each of said plurality of enhancers comprising a combination of the same and different enhancer sequences is non-sequentially arranged at 5′ to said core promoter in the recombinant polynucleotide.

Embodiment 36: The recombinant polynucleotide of embodiment 30, wherein each of said at least two enhancer sequences comprises (iii), wherein each of said plurality of enhancers comprising a combination of the same and different enhancer sequences is non-sequentially arranged at 5′ to said core promoter and at 3′ to said plurality of binding sites for one or more TFs, if present, in the recombinant polynucleotide.

Embodiment 37: The recombinant polynucleotide of any one of embodiments 3-36, wherein said plurality of enhancers comprises at least two EBS, C/EBP, ARE, DRE, NFκB, GC-box, UN5CL, BOP1, RTN4RL2, ARNTL2, AGR2, LHX2, TRNP1, MU5AC, or DOK4 enhancer sequences.

Embodiment 38: The recombinant polynucleotide of any one of embodiments 1-37, wherein expression of said ORF is increased when said recombinant polynucleotide is introduced to cancer cells compared to non-cancer cells.

Embodiment 39: The recombinant polynucleotide of any one of embodiments 1-37, wherein expression of said ORF is increased in a first plurality of cancer cells when said recombinant polynucleotide is introduced to said first plurality of cancer cells compared to a second plurality of cancer cells, wherein said first plurality of cancer cells and said second plurality of cancer cells are different types of cancer cells.

Embodiment 40: The recombinant polynucleotide of embodiment 38 or 39, wherein said cancer cells comprise malignant cancer cells.

Embodiment 41: The recombinant polynucleotide of any one of embodiments 38-40, wherein said cancer cells comprise lung cancer cells, colorectal cancer cells, breast cancer cells, or hepatocellular carcinoma cells.

Embodiment 42: The recombinant polynucleotide of any one of embodiments 38-40, wherein said cancer cells comprise cells associated with colorectal cancer, hepatocellular carcinoma, lung cancer, liver cancer, breast cancer, prostate cancer, cervix cancer, uterus cancer, pancreas cancer, kidney cancer, stomach cancer, bladder cancer, ovary cancer, brain cancer, head and neck cancer, eye cancer, mouth cancer, throat cancer, esophagus cancer, chest cancer, bone cancer, rectum or other gastrointestinal tract organ cancer, spleen cancer, skeletal muscle cancer, subcutaneous tissue cancer, testicles or other reproductive organ cancer, skin cancer, thyroid cancer, blood cancer, or lymph nodes cancer.

Embodiment 43: The recombinant polynucleotide of embodiment 42, wherein said cancer cells comprise cells associated with two or more cancers comprising colorectal cancer, hepatocellular carcinoma, lung cancer, liver cancer, breast cancer, prostate cancer, cervix cancer, uterus cancer, pancreas cancer, kidney cancer, stomach cancer, bladder cancer, ovary cancer, brain cancer, head and neck cancer, eye cancer, mouth cancer, throat cancer, esophagus cancer, chest cancer, bone cancer, rectum or other gastrointestinal tract organ cancer, spleen cancer, skeletal muscle cancer, subcutaneous tissue cancer, testicles or other reproductive organ cancer, skin cancer, thyroid cancer, blood cancer, or lymph nodes cancer.

Embodiment 44: The recombinant polynucleotide of any one of embodiments 3-43, wherein said core promoter, said plurality of binding sites for one or more transcription factors (TFs), said plurality of enhancers, or said recombinant polynucleotide comprises a sequence from Table 1A, Table 1B, or Table 1C.

Embodiment 45: A recombinant polynucleotide comprising any of the sequences from Table 1A, Table 1B, or Table 1C.

Embodiment 46: A recombinant polynucleotide comprising a human alpha-fetoprotein (AFP) promoter sequence comprising a plurality of HNF-1A TF binding sites, wherein each HNF-1A binding site comprises the sequence 5′-GTTAATTATTAAC-3′ (SEQ ID NO: 128).

Embodiment 47: A vector comprising the recombinant polynucleotide of any one of embodiments 1-46.

Embodiment 48: A pharmaceutical composition comprising the recombinant polynucleotide of any one of embodiments 1-46 or the vector of embodiment 47 and a pharmaceutically acceptable excipient, carrier, or diluents.

Embodiment 49: A lipid nanoparticle (LNP) comprising the recombinant polynucleotide of any one of embodiments 1-46, the vector of embodiment 47, or the pharmaceutical composition of embodiment 48.

Embodiment 50: A cell comprising the recombinant polynucleotide of any one of embodiments 1-46, the vector of embodiment 47, the pharmaceutical composition of embodiment 48, or the LNP of embodiment 49.

Embodiment 51: A method of selectively expressing a reporter protein in a cancer or tumor cell, comprising contacting said tumor cell the recombinant polynucleotide according to any one of embodiments 1-46, the vector of embodiment 47, the pharmaceutical composition of embodiment 48, or the LNP of embodiment 49, wherein the recombinant polynucleotide further comprises an open reading frame (ORF) encoding said reporter protein, wherein said ORF is operatively linked to said synthetic promoter.

Embodiment 52: A method comprising:
(a) administering to a subject the pharmaceutical composition of embodiment 48; or a composition comprising the recombinant polynucleotide of any one of embodiments 1-46, the vector of embodiment 47, or the LNP of embodiment 49; wherein the recombinant polynucleotide further comprises an open reading frame (ORF) encoding a reporter protein, wherein said ORF is operatively linked to a synthetic promoter in said recombinant polynucleotide, and
(b) detecting said reporter protein,
wherein said pharmaceutical composition or said composition induces expression of said reporter protein preferentially in diseased cells in said subject compared to in non-disease cells, and wherein a relative ratio of said reporter protein expressed in said diseased cells over said non-diseased cells is greater than 1.0.

Embodiment 53: The method of embodiment 52, wherein said relative ratio of said reporter protein expressed in said diseased cells over said non-diseased cells is greater than 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, or about 15.0, 20.0, 25.0, 30.0, 35.0, 40.0, 45.0, 50.0, 55.0, 60.0, 65.0, 70.0, 75.0, 80.0, 85.0, 90.0, 95.0, or about 100.0.

Embodiment 54: A method for treating a subject having or suspected of having a disease, comprising administering to said subject the pharmaceutical composition of embodiment 48; or a composition comprising the recombinant polynucleotide of any one of embodiments 1-46, the vector of embodiment 47, or the LNP of embodiment 49;
wherein the recombinant polynucleotide further comprises an open reading frame (ORF) encoding a therapeutic protein, wherein said ORF is operatively linked to a synthetic promoter in said recombinant polynucleotide, wherein said pharmaceutical composition or said composition induces expression of said therapeutic protein preferentially in diseased cells in said subject compared to in non-disease cells, and wherein a relative ratio of said therapeutic protein expressed in said diseased cells over said non-diseased cells is greater than 1.0.

Embodiment 55: The method of any one of embodiments 52-54, wherein said diseased cells comprise a cancer or tumor cell.

Embodiment 56: The method of embodiment 51 or 55, wherein said cancer or tumor cell is associated with colorectal cancer (CRC), hepatocellular carcinoma, lung cancer, liver cancer, breast cancer, prostate cancer, cervix cancer, uterus cancer, pancreas cancer, kidney cancer, stomach cancer, bladder cancer, ovary cancer, brain cancer, head and neck cancer, eye cancer, mouth cancer, throat cancer, esophagus cancer, chest cancer, bone cancer, rectum or other gastrointestinal tract organ cancer, spleen cancer, skeletal muscle cancer, subcutaneous tissue cancer, testicles or other reproductive organ cancer, skin cancer, thyroid cancer, blood cancer, or lymph nodes cancer.

Embodiment 57: A method comprising:
(a) administering to a subject the pharmaceutical composition of embodiment 48; or a composition comprising the recombinant polynucleotide of any one of embodiments 1-46, the vector of embodiment 47, or the LNP of embodiment 49; wherein said recombinant polynucleotide further comprises an open reading frame (ORF) encoding a reporter protein, wherein said ORF is operatively linked to a synthetic promoter in said recombinant polynucleotide, and
(b) localizing a tumor or an absence thereof in a body of said subject via expression of said reporter protein using an imaging technique performed on said body of said subject.

Embodiment 58: A method comprising:
(a) introducing to a subject suspected of having a cancer via intravenous administration the pharmaceutical composition of embodiment 48; or a composition comprising the recombinant polynucleotide of any one of embodiments 1-46, the vector of embodiment 47, or the LNP of embodiment 49; wherein said recombinant polynucleotide further comprises an open reading frame (ORF) encoding a reporter protein, wherein said ORF is operatively linked to a synthetic promoter in said recombinant polynucleotide, and
(b) detecting said reporter protein from said subject.

Embodiment 59: A method comprising:
(a) introducing to a subject suspected of having a cancer via intravenous administration a plurality of recombinant polynucleotides, wherein:
said plurality of recombinant polynucleotides comprises a plurality of different promoters of genes overexpressed in a tumor cell versus a normal tissue or functional fragments thereof operably linked to genes encoding reporter proteins, wherein said plurality of different promoters of genes overexpressed in said tumor cell versus said normal tissue drive expression of said corresponding reporter proteins in a cell affected by said cancer, wherein said DNA molecules are selected from the group consisting of nanoplasmids and linear double-stranded DNA molecules; and
(b) detecting said reporter proteins from said subject.

What is claimed is:

1. A recombinant double-stranded polynucleotide comprising:
(a) a core promoter comprising a transcription start site (TSS), wherein said core promoter is derived from one or more cancer-responsive genes that are expressed at a higher level in cancer cells compared to non-cancer cells and is operably linked to an open reading frame (ORF);
(b) a plurality of enhancers; and
(c) said ORF,
wherein: (a) said core promoter, and (b) said plurality of enhancers are not derived from a same gene.

2. The recombinant double-stranded polynucleotide of claim 1, wherein said core promoter further comprises two or more promoter elements derived from two or more cancer-responsive genes that are either expressed at a higher level in cancer cells compared to non-cancer cells and are operatively linked to said ORF.

3. The recombinant double-stranded polynucleotide of claim 1, wherein said one or more cancer-responsive genes are derived from a human subject.

4. The recombinant double-stranded polynucleotide of claim 1, wherein at least one of said plurality of enhancers comprises a CpG island.

5. The recombinant double-stranded polynucleotide of claim 1, wherein at least one of said plurality of enhancers does not comprise a CpG island.

6. The recombinant double-stranded polynucleotide of claim 1, wherein said higher levels of expression in said cancer cells compared to non-cancer cells are determined by chromatin immunoprecipitation (ChIP).

7. The recombinant double-stranded polynucleotide of claim 1, wherein said ORF encodes a reporter protein, a biomarker protein, or a therapeutic protein.

8. The recombinant double-stranded polynucleotide of claim 1, further comprising a spacer element comprising 1-20 nucleotides between each of said plurality of enhancers.

9. The recombinant double-stranded polynucleotide of claim 1, wherein said one or more cancer-responsive genes from which said core promoter is derived comprise one or more of TCF7, MNX1, HOXC10, TP53, CEACAM5, CEP55, FAM111B, CST1, BIRC5, FOS, TWIST1, E2F2, KIF20A, or ETV4.

10. The recombinant double-stranded polynucleotide of claim 1, wherein said one or more cancer-responsive genes from which said core promoter is derived comprise FAM111B and KIF20A.

11. The recombinant double-stranded polynucleotide of claim 1, wherein said one or more cancer-responsive genes from which said core promoter is derived comprise FAM111B.

12. The recombinant double-stranded polynucleotide of claim 1, wherein said core promoter comprises a sequence from a region from about −300 bp to +100 bp relative to a TSS of said one or more cancer-responsive genes.

13. The recombinant double-stranded polynucleotide of claim 1, wherein each of said plurality of enhancer sequences comprises different enhancer sequences.

14. The recombinant double-stranded polynucleotide of claim 13, wherein each of said at plurality of enhancer sequences is sequentially arranged at 5′ to said core promoter in said recombinant double-stranded polynucleotide.

15. The recombinant double-stranded polynucleotide of claim 1, wherein said cancer cells comprise cells of colorectal cancer, hepatocellular carcinoma, lung cancer, liver cancer, breast cancer, prostate cancer, cervix cancer, uterus cancer, pancreas cancer, kidney cancer, stomach cancer, bladder cancer, ovary cancer, brain cancer, head and neck cancer, eye cancer, mouth cancer, throat cancer, esophagus cancer, chest cancer, bone cancer, rectum or other gastrointestinal tract organ cancer, spleen cancer, skeletal muscle cancer, subcutaneous tissue cancer, testicles or other reproductive organ cancer, skin cancer, thyroid cancer, blood cancer, or lymph node cancer.

16. The recombinant double-stranded polynucleotide of claim 1, wherein an enhancer of said plurality of enhancers comprises a sequence having at least 80% sequence identity to any one of SEQ ID NOs: 377-397, or a reverse complement thereof.

17. The recombinant double-stranded polynucleotide of claim 1, wherein an enhancer of said plurality of enhancers comprises a sequence having at least 80% sequence identity to at least one of SEQ ID NOs: 386, 388, or 384, or a reverse complement thereof.

18. The recombinant double-stranded polynucleotide of claim 1, wherein said core promoter comprises at least about 35 consecutive nucleotides having at least 80% sequence identity to any one of SEQ ID NOs: 558-584, or a reverse complement thereof.

19. The recombinant double-stranded polynucleotide of claim 1, wherein said core promoter comprises at least about 35 consecutive nucleotides having at least 80% sequence identity to any one of SEQ ID NO: 560 or SEQ ID NO: 570.

20. The recombinant double-stranded polynucleotide of claim 1, wherein said core promoter comprises a sequence having at least 80% sequence identity to any one of SEQ ID NOs: 584-587.

21. A lipid nanoparticle (LNP) comprising the recombinant double-stranded polynucleotide of claim 1.

22. A method of selectively expressing a protein in a cancer cell, comprising contacting said cancer cell with the recombinant double-stranded polynucleotide of claim 1.

23. The method of claim 22, wherein said method is selective for expression of said protein in said cancer cell versus a non-cancer cell.

24. The method of claim 22, wherein said contacting comprises intravenous or intratumoral administration to a subject.

25. The method of claim 24, wherein said subject has lung, liver, ovarian, breast, muscle, bladder, colorectal, kidney, stomach, uterus, skin, or bone cancer

26. A method of expressing a protein in a subject, comprising intravenously administering to said subject the recombinant double-stranded polynucleotide of claim 1.

27. The method of claim 26, wherein said subject has or is suspected of having a cancer.
